(19) United States
(12) Patent Application Publication
(10) Pub. No.: US 2022/0184131 A1
(43) Pub. Date: Jun. 16, 2022

(54) CELLS EXPRESSING A RECOMBINANT RECEPTOR FROM A MODIFIED TGFBR2 LOCUS, RELATED POLYNUCLEOTIDES AND METHODS

(71) Applicants: Juno Therapeutics, Inc., Seattle, WA (US); Editas Medicine, Inc., Cambridge, MA (US)

(72) Inventors: Stephen Michael Burleigh, Seattle, WA (US); Cedric CLEYRAT, Seattle, WA (US); Melissa CHIN, Cambridge, MA (US); Fred HARBINSKI, Cambridge, MA (US); Christopher Heath NYE, Seattle, WA (US); Blythe D. SATHER, Seattle, WA (US); Queenie VONG, Seattle, WA (US); G. GRANT WELSTEAD, Cambridge, MA (US); Christopher WILSON, Cambridge, MA (US)

(73) Assignees: Juno Therapeutics, Inc., Seattle, WA (US); Editas Medicine, Inc., Cambridge, MA (US)

(21) Appl. No.: 17/607,816

(22) PCT Filed: Apr. 30, 2020
(86) PCT No.: PCT/US2020/030815

§ 371 Date: Oct. 29, 2021

Related U.S. Application Data
(60) Provisional application No. 62/841,575, filed on May 1, 2019.

(51) Int. Cl. A61K 35/17; C12N 5/0783; C07K 14/71; C07K 14/725; C07K 14/705; C12N 15/86; C12N 15/11; C12N 9/22; A61P 35/00; C07K 16/28

(52) U.S. Cl. CPC A61K 35/17; A61K 38/00; C07K 14/71; C07K 14/7051; C07K 14/70521; C07K 14/70578; C12N 15/86; C12N 15/11; C12N 9/22; A61P 35/00; C07K 16/2803; C12N 2510/00; C07K 2319/03; C07K 2319/30; C07K 2319/33; C07K 2317/524; C07K 2317/526; C07K 2317/53; C07K 2319/02; C12N 2750/14143; C12N 2310/20; C12N 2800/80; C12N 5/0636

(57) ABSTRACT

Provided herein are engineered immune cells, e.g. T cells, expressing a recombinant receptor, that contain a modified transforming growth factor-beta receptor type-2 (TGFBR2) locus encoding the recombinant receptor or a portion thereof. In some aspects, the cells are engineered by targeted integration of a transgene sequence encoding the recombinant receptor or a portion thereof, at a TGFBR2 genomic locus. Also provided are cell compositions containing the engineered immune cells, nucleic acids for engineering cells, and methods, kits and articles of manufacture for producing the engineered cells, such as by targeting a transgene sequence encoding a recombinant receptor or a portion thereof for integration into a region of a TGFBR2 genomic locus. In some embodiments, the engineered cells, e.g. T cells, can be used in connection with cell therapy, including in connection with cancer immunotherapy comprising adoptive transfer of the engineered cells.

### CROSS-REFERENCE TO RELATED APPLICATIONS

This application claims priority from U.S. provisional application No. 62/841,575, filed May 1, 2019, entitled “CELLS EXPRESSING A RECOMBINANT RECEPTOR FROM A MODIFIED TGFBR2 LOCUS, RELATED POLYNUCLEOTIDES AND METHODS,” the contents of which are incorporated by reference in their entirety.

### INCORPORATION BY REFERENCE OF SEQUENCE LISTING

The present application is being filed along with a Sequence Listing in electronic format. The Sequence Listing is provided as a file entitled 735042012840SeqList.txt, created Apr. 28, 2020, which is 200 kilobytes in size. The information in the electronic format of the Sequence Listing is incorporated by reference in its entirety.

### FIELD

The present disclosure relates to engineered immune cells, e.g. T cells, expressing a recombinant receptor, that contain a modified transforming growth factor-beta receptor type-2 (TGFBR2) locus encoding the recombinant receptor or a portion thereof. In some aspects, the cells are engineered by targeted integration of a transgene sequence encoding the recombinant receptor or a portion thereof, at a TGFBR2 genomic locus. Also disclosed are cell compositions containing the engineered immune cells, nucleic acids for engineering cells, and methods, kits and articles of manufacture for producing the engineered cells, such as by targeting a transgene sequence encoding a recombinant receptor or a portion thereof for integration into a region of a TGFBR2 genomic locus. In some embodiments, the engineered cells, e.g. T cells, can be used in connection with cell therapy, including in connection with cancer immunotherapy comprising adoptive transfer of the engineered cells.

### BACKGROUND

Adoptive cell therapies that utilize recombinant receptors, such as chimeric antigen receptors (CARs), to recognize antigens associated with a disease represent an attractive therapeutic modality for the treatment of cancers and other diseases. Improved strategies are needed for engineering T cells to express recombinant receptors, such as for use in adoptive immunotherapy, e.g., in treating cancer, infectious diseases and autoimmune diseases. Provided are methods, cells, compositions and kits for use in the methods that meet such needs.

### SUMMARY

Provided herein are genetically engineered T cells and compositions, methods, uses, kits, and articles of manufacture related to genetically engineered T cells. In some of any of the provided embodiments, the genetically engineered T cell comprises a modified transforming growth factor-beta receptor type-2 (TGFBR2) locus. In some of any embodiments, the modified TGFBR2 locus comprises a transgene sequence encoding a recombinant receptor or a portion thereof.

Provided herein are genetically engineered T cells that contain a modified transforming growth factor-beta receptor type-2 (TGFBR2) locus, said modified TGFBR2 locus comprising a transgene sequence encoding a recombinant receptor or a portion thereof. In some of any embodiments, the transgene sequence has been integrated at the endogenous TGFBR2 locus. In some of any embodiments, the integration is via homology directed repair (HDR).

In some of any embodiments, the modified TGFBR2 locus does not encode a functional TGFBRII polypeptide. In some of any embodiments, the modified TGFBR2 locus does not encode a TGFBRII polypeptide or the expression of TGFBRII polypeptide is eliminated. In some of any embodiments, the modified TGFBR2 locus does not encode a full length TGFBRII polypeptide or encodes a partial TGFBRII polypeptide. In some of any embodiments, the modified TGFBR2 locus encodes a dominant negative TGFBRII polypeptide. In some of any embodiments, the encoded TGFBRII polypeptide comprises an amino acid sequence corresponding to residues 22-191 of SEQ ID NO:59 or residues 22-216 of SEQ ID NO:60 In some of any embodiments, the encoded TGFBRII polypeptide comprises a sequence that exhibits at least 85%, 86%, 87%, 88%, 89%, 90%, 91%, 92%, 93%, 94%, 95%, 96%, 97%, 98%, 99% or more sequence identity to an amino acid sequence corresponding to residues 22-191 of SEQ ID NO:59 or residues 22-216 of SEQ ID NO:60 or a fragment thereof. In some of any embodiments, the transgene sequence is in-frame with one or more exons of an open reading frame or partial sequence thereof of the endogenous TGFBR2 locus.

In some of any embodiments, the transgene sequence is downstream of exon 1 and upstream of exon 6 of the open reading frame of the endogenous TGFBR2 locus. In some of any embodiments, the transgene sequence is downstream of exon 4 and upstream of exon 6 of the open reading frame of the endogenous TGFBR2 locus.

In some of any embodiments, the recombinant receptor is or comprises recombinant T cell receptor (TCR). In some of any embodiments, the recombinant receptor is a recombinant TCR and the transgene sequence encodes a TCR alpha (TCRα) chain, a TCR beta (TCRβ) chain or both. In some of any embodiments, the recombinant receptor is a functional non-T cell receptor (non-TCR) antigen receptor. In some of any embodiments, the recombinant receptor comprises a functional non-T cell receptor (non-TCR) antigen receptor. In some of any embodiments, the recombinant receptor is a chimeric antigen receptor (CAR). In some of any embodiments, the CAR comprises an extracellular region, a transmembrane domain, and an intracellular region. In some of any embodiments, the extracellular region comprises a binding domain. In some of any embodiments, the binding domain is an antibody or an antigen-binding fragment thereof. In some of any embodiments, the binding domain comprises an antibody or an antigen-binding fragment thereof. In some of any embodiments, the binding domain is capable of binding to a target antigen that is associated with, specific to, or expressed on a cell or tissue of a disease, disorder or condition.

In some of any embodiments, the target antigen is a tumor antigen. In some of any embodiments, the target antigen is selected from among αvβ6 integrin (avb6 integrin), B cell maturation antigen (BCMA), B7-H3, B7-H6, carbonic anhydrase 9 (CA9, also known as CAIX or G250), a cancer-testis antigen, cancer/testis antigen 1B (CTAG, also known as NY-ESO-1 and LAGE-2), carcinoembryonic antigen (CEA), a cyclin, cyclin A2, C—C Motif Chemokine Ligand 1 (CCL-1), CD19, CD20, CD22, CD23, CD24, CD30, CD33, CD38, CD44, CD44v6, CD44v7/8, CD123, CD133, CD138, CD171, chondroitin sulfate proteoglycan 4 (CSPG4), epidermal growth factor protein (EGFR), type III epidermal growth factor receptor mutation (EGFR vIII), epithelial glycoprotein 2 (EPG-2), epithelial glycoprotein 40 (EPG-40), ephrinB2, ephrin receptor A2 (EPHa2), estrogen receptor, Fc receptor like 5 (FCRL5; also known as Fc receptor homolog 5 or FCRH5), fetal acetylcholine receptor (fetal AchR), a folate binding protein (FBP), folate receptor alpha, ganglioside GD2, O-acetylated GD2 (OGD2), ganglioside GD3, glycoprotein 100 (gp100), glypican-3 (GPC3), G protein-coupled receptor class C group 5 member D (GPRC5D), Her2/neu (receptor tyrosine kinase erb-B2), Her3 (erb-B3), Her4 (erb-B4), erbB dimers, Human high molecular weight-melanoma-associated antigen (HMW-MAA), hepatitis B surface antigen, Human leukocyte antigen A1 (HLA-A1), Human leukocyte antigen A2 (HLA-A2), IL-22 receptor alpha (IL-22Rα), IL-13 receptor alpha 2 (IL-13Rα2), kinase insert domain receptor (kdr), kappa light chain, L1 cell adhesion molecule (L1-CAM), CE7 epitope of L1-CAM, Leucine Rich Repeat Containing 8 Family Member A (LRRC8A), Lewis Y, Melanoma-associated antigen (MAGE)-A1, MAGE-A3, MAGE-A6, MAGE-A10, mesothelin (MSLN), c-Met, murine cytomegalovirus (CMV), mucin 1 (MUC1), MUC16, natural killer group 2 member D (NKG2D) ligands, melan A (MART-1), neural cell adhesion molecule (NCAM), oncofetal antigen, Preferentially expressed antigen of melanoma (PRAME), progesterone receptor, a prostate specific antigen, prostate stem cell antigen (PSCA), prostate specific membrane antigen (PSMA), Receptor Tyrosine Kinase Like Orphan Receptor 1 (ROR1), survivin, Trophoblast glycoprotein (TPBG also known as 5T4), tumor-associated glycoprotein 72 (TAG72), Tyrosinase related protein 1 (TRP1, also known as TYRP1 or gp75), Tyrosinase related protein 2 (TRP2, also known as dopachrome tautomerase, dopachrome delta-isomerase or DCT), vascular endothelial growth factor receptor (VEGFR), vascular endothelial growth factor receptor 2 (VEGFR2), Wilms Tumor 1 (WT-1), a pathogen-specific or pathogen-expressed antigen, or an antigen associated with a universal tag, and/or biotinylated molecules, and/or molecules expressed by HIV, HCV, HBV or other pathogens.

In some of any embodiments, the extracellular region comprises a spacer. In some of any embodiments, the spacer is operably linked between the binding domain and the transmembrane domain. In some of any embodiments, the spacer comprises an immunoglobulin hinge region. In some of any embodiments, the spacer comprises a C_{H}2 region and a C_{H}3 region. In some of any embodiments, the intracellular region comprises an intracellular signaling domain. In some of any embodiments, the intracellular signaling domain is an intracellular signaling domain of a CD3 chain, such as a CD3-zeta (CD3) chain, or a signaling portion thereof. In some of any embodiments, the intracellular signaling domain comprises an intracellular signaling domain of a CD3 chain, such as a CD3-zeta (CD3) chain, or a signaling portion thereof. In some of any embodiments, the intracellular region comprises one or more costimulatory signaling domain(s). In some of any embodiments, the one or more costimulatory signaling domain comprises an intracellular signaling domain of a CD28, a 4-1BB or an ICOS or a signaling portion thereof. In some of any embodiments, the costimulatory signaling region comprises an intracellular signaling domain of 4-1BB.

In some of any embodiments, the modified TGFBR2 locus encodes a recombinant receptor that comprises, from its N to C terminus in order: the extracellular binding domain, the spacer, the transmembrane domain and an intracellular signaling region.

In some of any embodiments, the transgene sequence comprises in order a sequence of nucleotides encoding an extracellular binding domain; a spacer; and a transmembrane domain; a costimulatory signaling domain; and an intracellular signaling region. In some of any embodiments, the modified TGFBR2 locus comprises in order: a sequence of nucleotides encoding an extracellular binding domain; a spacer; and a transmembrane domain; a costimulatory signaling domain; and an intracellular signaling region.

In some of any embodiments, the transgene sequence comprises in order a sequence of nucleotides encoding an extracellular binding domain, that is an scFv; a spacer, that comprises a sequence from a human immunoglobulin hinge, that is an IgG1, IgG2 or IgG4 or a modified version thereof, that further comprises a C_{H}2 region and/or a C_{H}3 region; and a transmembrane domain, that is from human CD28; a costimulatory signaling domain, that is from human 4-1BB; and an intracellular signaling region, that is a CD3ζ chain or a portion thereof. In some of any embodiments, the modified TGFBR2 locus comprises in order: a sequence of nucleotides encoding an extracellular binding domain, that is an scFv; a spacer, that comprises a sequence from a human immunoglobulin hinge, that is from IgG1, IgG2 or IgG4 or a modified version thereof, that further comprises a C_{H}2 region and/or a C_{H}3 region; and a transmembrane domain, that is from human CD28; a costimulatory signaling domain, that is from human 4-1BB; and an intracellular signaling region, that is a CD3ζ chain or a portion thereof.

In some of any embodiments, the CAR is a multi-chain CAR. In some of any embodiments, the transgene sequence comprises a sequence of nucleotides encoding at least one further protein.

In some of any embodiments, the transgene sequence comprises one or more multicistronic element(s). In some of any embodiments, the one or more multicistronic element is positioned between the sequence of nucleotides encoding the CAR and the sequence of nucleotides encoding the at least one further protein. In some of any embodiments, the at least one further protein is a surrogate marker. In some of any embodiments, the surrogate marker is a truncated receptor. In some of any embodiments, the truncated receptor lacks an intracellular signaling domain and is not capable of mediating intracellular signaling when bound by its ligand. In some of any embodiments, the truncated receptor lacks an intracellular signaling domain or is not capable of mediating intracellular signaling when bound by its ligand.

In some of any embodiments, the recombinant receptor is a recombinant TCR, and a multicistronic element is positioned between a sequence of nucleotides encoding the TCRα and a sequence of nucleotides encoding the TCRβ.

In some of any embodiments, the recombinant receptor is a multi-chain CAR, and a multicistronic element is positioned between a sequence of nucleotides encoding one chain of the multi-chain CAR and a sequence of nucleotides encoding another chain of the multi-chain CAR.

In some of any embodiments, the one or more multicistronic element(s) are upstream of the sequence of nucleotides encoding the recombinant receptor.

In some of any embodiments, the one or more multicistronic element is or comprises a ribosome skip sequence. In some of any embodiments, the ribosome skip sequence is a T2A, a P2A, an E2A, or an F2A element.

In some of any embodiments, the modified TGFBR2 locus comprises the promoter and regulatory or control element of the endogenous TGFBR2 locus operably linked to control expression the nucleic acid sequence encoding the recombinant receptor. In some of any embodiments, the modified TGFBR2 locus comprises the promoter or regulatory or control element of the endogenous TGFBR2 locus operably linked to control expression the nucleic acid sequence encoding the recombinant receptor. In some of any embodiments, the modified locus comprises one or more heterologous regulatory or control element(s) operably linked to control expression of the nucleic acid sequence encoding the recombinant receptor. In some of any embodiments, the one or more heterologous regulatory or control element comprises a heterologous promoter, an enhancer, an intron, a polyadenylation signal, a Kozak consensus sequence, a splice acceptor sequence or a splice donor sequence. In some of any embodiments, the heterologous promoter is or comprises a human elongation factor 1 alpha (EF1α) promoter or an MND promoter or a variant thereof.

In some of any embodiments, the T cell is a primary T cell derived from a subject. In some of any embodiments, the subject is a human. In some of any embodiments, the T cell is a CD8+ T cell or subtypes thereof. In some of any embodiments, the T cell is a CD4+ T cell or subtypes thereof. In some of any embodiments, the T cell is derived from a multipotent or pluripotent cell. In some of any embodiments, the T cell is derived from a multipotent or pluripotent cell, which is an iPSC.

Provided herein are polynucleotides, comprising a nucleic acid sequence encoding a recombinant receptor or a portion thereof; and one or more homology arm(s) linked to the nucleic acid sequence. In some of any embodiments, the one or more homology arm(s) comprise a sequence homologous to one or more region(s) of an open reading frame of a transforming growth factor-beta receptor type-2 (TGFBR2) locus. In some of any embodiments, the recombinant receptor or a portion thereof is encoded by a modified TGFBR2 locus comprising the nucleic acid sequence encoding the recombinant receptor or a portion thereof when the recombinant receptor is expressed from a cell introduced with the polynucleotide. In some of any embodiments, the nucleic acid sequence is a sequence that is exogenous or heterologous to an open reading frame of the endogenous genomic TGFBR2 locus a T cell. In some of any embodiments, the nucleic acid sequence is a sequence that is exogenous or heterologous to an open reading frame of the endogenous genomic TGFBR2 locus a T cell, which is a human T cell.

In some of any embodiments, the one or more homology arm(s) comprise at least one intron or at least one exon of the open reading frame of the TGFBR2 locus. In some of any embodiments, the modified TGFBR2 locus does not encode a functional TGFBRII polypeptide, in a cell introduced with the polynucleotide. In some of any embodiments, the modified TGFBR2 locus does not encode a TGFBRII polypeptide or the expression of TGFBRII polypeptide is eliminated, in a cell introduced with the polynucleotide.

In some of any embodiments, the modified TGFBR2 locus does not encode a full length TGFBRII polypeptide or encodes a partial TGFBRII polypeptide, in a cell introduced with the polynucleotide. In some of any embodiments, the modified TGFBR2 locus encodes a dominant negative TGFBRII polypeptide, in a cell introduced with the polynucleotide. In some of any embodiments, the encoded TGFBRII polypeptide in a cell introduced with the polynucleotide comprises an amino acid sequence corresponding to residues 22-191 of SEQ ID NO:59 or residues 22-216 of SEQ ID NO:60 or a sequence that exhibits at least 85%, 86%, 87%, 88%, 89%, 90%, 91%, 92%, 93%, 94%, 95%, 96%, 97%, 98%, 99% or more sequence identity to an amino acid sequence corresponding to residues 22-191 of SEQ ID NO:59 or residues 22-216 of SEQ ID NO:60 or a fragment thereof. In some of any embodiments, the nucleic acid sequence is in-frame with one or more exons of the open reading frame of the TGFBR2 locus comprised in the one or more homology arm(s).

In some of any embodiments, the one or more region(s) of the open reading frame is or comprises sequences that are downstream of exon 1 of the open reading frame of the endogenous TGFBR2 locus. In some of any embodiments, the one or more region(s) of the open reading frame is or comprises sequences that includes at least a portion of exon 4 or downstream of exon 4 of the open reading frame of the TGFBR2 locus.

In some of any embodiments, the one or more homology arm comprises a 5′ homology arm and a 3′ homology arm. In some of any embodiments, the polynucleotide comprises the structure [5′ homology arm]-[nucleic acid sequence of (a)]-[3′ homology arm]. In some of any embodiments, the 5′ homology arm and the 3′ homology arm independently are from at or about 50 to at or about 2000 nucleotides, from at or about 100 to at or about 1000 nucleotides, from at or about 100 to at or about 750 nucleotides, from at or about 100 to at or about 600 nucleotides, from at or about 100 to at or about 400 nucleotides, from at or about 100 to at or about 300 nucleotides, from at or about 100 to at or about 200 nucleotides, from at or about 200 to at or about 1000 nucleotides, from at or about 200 to at or about 750 nucleotides, from at or about 200 to at or about 600 nucleotides, from at or about 200 to at or about 400 nucleotides, from at or about 200 to at or about 300 nucleotides, from at or about 300 to at or about 1000 nucleotides, from at or about 300 to at or about 750 nucleotides, from at or about 300 to at or about 600 nucleotides, from at or about 300 to at or about 400 nucleotides, from at or about 400 to at or about 1000 nucleotides, from at or about 400 to at or about 750 nucleotides, from at or about 400 to at or about 600 nucleotides, from at or about 600 to at or about 1000 nucleotides, from at or about 600 to at or about 750 nucleotides or from at or about 750 to at or about 1000 nucleotides in length. In some of any embodiments, the 5′ homology arm and the 3′ homology arm independently are at or about 200, 300, 400, 500, 600, 700 or 800 nucleotides in length, or any value between any of the foregoing. In some of any embodiments, the 5′ homology arm and the 3′ homology arm independently are greater than at or about 300 nucleotides in length. In some of any embodiments, the 5′ homology arm and the 3′ homology arm independently are at or about 400, 500 or 600 nucleotides in length, or any value between any of the foregoing.

In some of any embodiments, the 5′ homology arm comprises the sequence set forth in SEQ ID NOS: 69-71 or a sequence that exhibits at least 85%, 86%, 87%, 88%, 89%, 90%, 91%, 92%, 93%, 94%, 95%, 96%, 97%, 98%, 99% or more sequence identity to SEQ ID NOS: 69-71 or a partial sequence thereof. In some of any embodiments, the 3′ homology arm comprises the sequence set forth in SEQ ID NO:72, or a sequence that exhibits at least 85%, 86%, 87%, 88%, 89%, 90%, 91%, 92%, 93%, 94%, 95%, 96%, 97%, 98%, 99% or more sequence identity to SEQ ID NO:72 or a partial sequence thereof.

In some of any embodiments, the encoded recombinant receptor is or comprises recombinant T cell receptor (TCR). In some of any embodiments, the encoded recombinant receptor is a recombinant TCR and the nucleic acid sequence in (a) encodes a TCR alpha (TCRα) chain, a TCR beta (TCRβ) chain or both.

In some of any embodiments, the encoded recombinant receptor is a functional non-T cell receptor (non-TCR) antigen receptor. In some of any embodiments, the encoded recombinant receptor comprises a functional non-T cell receptor (non-TCR) antigen receptor. In some of any embodiments, the encoded recombinant receptor is a chimeric antigen receptor (CAR).

In some of any embodiments, the CAR comprises an extracellular region, a transmembrane domain, and an intracellular region. In some of any embodiments, the extracellular region comprises a binding domain. In some of any embodiments, the binding domain is an antibody or an antigen-binding fragment thereof. In some of any embodiments, the binding domain comprises an antibody or an antigen-binding fragment thereof. In some of any embodiments, the binding domain is capable of binding to a target antigen that is associated with, specific to, or expressed on a cell or tissue of a disease, disorder or condition.

In some of any embodiments, the target antigen is a tumor antigen. In some of any embodiments, the target antigen is selected from among αvβ6 integrin (avb6 integrin), B cell maturation antigen (BCMA), B7-H3, B7-H6, carbonic anhydrase 9 (CA9, also known as CAIX or G250), a cancer-testis antigen, cancer/testis antigen 1B (CTAG, also known as NY-ESO-1 and LAGE-2), carcinoembryonic antigen (CEA), a cyclin, cyclin A2, C—C Motif Chemokine Ligand 1 (CCL-1), CD19, CD20, CD22, CD23, CD24, CD30, CD33, CD38, CD44, CD44v6, CD44v7/8, CD123, CD133, CD138, CD171, chondroitin sulfate proteoglycan 4 (CSPG4), epidermal growth factor protein (EGFR), type III epidermal growth factor receptor mutation (EGFR vIII), epithelial glycoprotein 2 (EPG-2), epithelial glycoprotein 40 (EPG-40), ephrinB2, ephrin receptor A2 (EPHa2), estrogen receptor, Fc receptor like 5 (FCRL5; also known as Fc receptor homolog 5 or FCRH5), fetal acetylcholine receptor (fetal AchR), a folate binding protein (FBP), folate receptor alpha, ganglioside GD2, O-acetylated GD2 (OGD2), ganglioside GD3, glycoprotein 100 (gp100), glypican-3 (GPC3), G protein-coupled receptor class C group 5 member D (GPRC5D), Her2/neu (receptor tyrosine kinase erb-B2), Her3 (erb-B3), Her4 (erb-B4), erbB dimers, Human high molecular weight-melanoma-associated antigen (HMW-MAA), hepatitis B surface antigen, Human leukocyte antigen A1 (HLA-A1), Human leukocyte antigen A2 (HLA-A2), IL-22 receptor alpha (IL-22Rα), IL-13 receptor alpha 2 (IL-13Rα2), kinase insert domain receptor (kdr), kappa light chain, L1 cell adhesion molecule (L1-CAM), CE7 epitope of L1-CAM, Leucine Rich Repeat Containing 8 Family Member A (LRRC8A), Lewis Y, Melanoma-associated antigen (MAGE)-A1, MAGE-A3, MAGE-A6, MAGE-A10, mesothelin (MSLN), c-Met, murine cytomegalovirus (CMV), mucin 1 (MUC1), MUC16, natural killer group 2 member D (NKG2D) ligands, melan A (MART-1), neural cell adhesion molecule (NCAM), oncofetal antigen, Preferentially expressed antigen of melanoma (PRAME), progesterone receptor, a prostate specific antigen, prostate stem cell antigen (PSCA), prostate specific membrane antigen (PSMA), Receptor Tyrosine Kinase Like Orphan Receptor 1 (ROR1), survivin, Trophoblast glycoprotein (TPBG also known as 5T4), tumor-associated glycoprotein 72 (TAG72), Tyrosinase related protein 1 (TRP1, also known as TYRP1 or gp75), Tyrosinase related protein 2 (TRP2, also known as dopachrome tautomerase, dopachrome delta-isomerase or DCT), vascular endothelial growth factor receptor (VEGFR), vascular endothelial growth factor receptor 2 (VEGFR2), Wilms Tumor 1 (WT-1), a pathogen-specific or pathogen-expressed antigen, or an antigen associated with a universal tag, and/or biotinylated molecules, and/or molecules expressed by HIV, HCV, HBV or other pathogens.

In some of any embodiments, the extracellular region comprises a spacer. In some of any embodiments, the extracellular region comprises a spacer which is operably linked between the binding domain and the transmembrane domain. In some of any embodiments, the spacer comprises an immunoglobulin hinge region. In some of any embodiments, the spacer comprises a C_{H}2 region and a C_{H}3 region. In some of any embodiments, the intracellular region comprises an intracellular signaling domain. In some of any embodiments, the intracellular signaling domain is an intracellular signaling domain of a CD3 chain. In some of any embodiments, the intracellular signaling domain is an intracellular signaling domain of a CD3 chain, which is a CD3-zeta (CD3) chain, or a signaling portion thereof. In some of any embodiments, the intracellular signaling domain comprises an intracellular signaling domain of a CD3 chain. In some of any embodiments, the intracellular signaling domain comprises an intracellular signaling domain of a CD3 chain, which is a CD3-zeta (CD3) chain, or a signaling portion thereof. In some of any embodiments, the intracellular region comprises one or more costimulatory signaling domain(s). In some of any embodiments, the one or more costimulatory signaling domain comprises an intracellular signaling domain of a CD28, a 4-1BB or an ICOS or a signaling portion thereof. In some of any embodiments, the costimulatory signaling region comprises an intracellular signaling domain of 4-1BB.

In some of any embodiments, the modified TGFBR2 locus encodes a recombinant receptor that comprises, from its N to C terminus in order: the extracellular binding domain, the spacer, the transmembrane domain and an intracellular signaling region. In some of any embodiments, the transgene sequence comprises in order a sequence of nucleotides encoding an extracellular binding domain; a spacer; and a transmembrane domain; and an intracellular signaling region.

In some of any embodiments, the transgene sequence comprises in order a sequence of nucleotides encoding an extracellular binding domain, that is an scFv; a spacer, that comprises a sequence from a human immunoglobulin hinge, that is from IgG1, IgG2 or IgG4 or a modified version thereof, that further comprises a C_{H}2 region and/or a C_{H}3 region; and a transmembrane domain, that is from human CD28; a costimulatory signaling domain, that is from human 4-1BB; and an intracellular signaling region, that is a CD3ζ chain or a portion thereof.

In some of any embodiments, the CAR is a multi-chain CAR. In some of any embodiments, the nucleic acid sequence comprises a sequence of nucleotides encoding at least one further protein.

In some of any embodiments, the nucleic acid sequence comprises one or more multicistronic element(s). In some of any embodiments, the one or more multicistronic element is positioned between the sequence of nucleotides encoding the CAR and the sequence of nucleotides encoding the at least one further protein.

In some of any embodiments, the at least one further protein is a surrogate marker. In some of any embodiments, the at least one further protein is a surrogate marker which is a truncated receptor. In some of any embodiments, the at least one further protein is a surrogate marker which is a truncated receptor which lacks an intracellular signaling domain and is not capable of mediating intracellular signaling when bound by its ligand. In some of any embodiments, the at least one further protein is a surrogate marker which is a truncated receptor which lacks an intracellular signaling domain or is not capable of mediating intracellular signaling when bound by its ligand.

In some of any embodiments, the recombinant receptor is a recombinant TCR, and a multicistronic element is positioned between a sequence of nucleotides encoding the TCRα and a sequence of nucleotides encoding the TCRβ.

In some of any embodiments, the recombinant receptor is a multi-chain CAR, and a multicistronic element is positioned between a sequence of nucleotides encoding one chain of the multi-chain CAR and a sequence of nucleotides encoding another chain of the multi-chain CAR.

In some of any embodiments, the one or more multicistronic element(s) are upstream of the sequence of nucleotides encoding the recombinant receptor. In some of any embodiments, the one or more multicistronic element is or comprises a ribosome skip sequence. In some of any embodiments, the one or more multicistronic element is or comprises a ribosome skip sequence which is a T2A, a P2A, an E2A, or an F2A element.

In some of any embodiments, the nucleic acid sequence comprises one or more heterologous or regulatory control element(s) operably linked to control expression of the recombinant receptor when expressed from a cell introduced with the polynucleotide. In some of any embodiments, the one or more heterologous regulatory or control element comprises a heterologous promoter, an enhancer, an intron, a polyadenylation signal, a Kozak consensus sequence, a splice acceptor sequence and/or a splice donor sequence. In some of any embodiments, the heterologous promoter is or comprises a human elongation factor 1 alpha (EF1α) promoter or an MND promoter or a variant thereof.

In some of any embodiments, the polynucleotide is comprised in a viral vector. In some of any embodiments, the viral vector is an AAV vector. In some of any embodiments, the AAV vector is selected from among AAV1, AAV2, AAV3, AAV4, AAV5, AAV6, AAV7 or AAV8 vector. In some of any embodiments, the AAV vector is an AAV2 or AAV6 vector. In some of any embodiments, the viral vector is a retroviral vector. In some of any embodiments, the viral vector is a retroviral vector which is a lentiviral vector.

In some of any embodiments, the polynucleotide is a linear polynucleotide. In some of any embodiments, the polynucleotide is a linear polynucleotide, which is a double-stranded polynucleotide or a single-stranded polynucleotide. In some of any embodiments, the polynucleotide is at least at or about 2500, 2750, 3000, 3250, 3500, 3750, 4000, 4250, 4500, 4760, 5000, 5250, 5500, 5750, 6000, 7000, 7500, 8000, 9000 or 10000 nucleotides in length, or any value between any of the foregoing. In some of any embodiments, the polynucleotide is between at or about 2500 and at or about 5000 nucleotides, at or about 3500 and at or about 4500 nucleotides, or at or about 3750 nucleotides and at or about 4250 nucleotides in length.

Provided herein are methods of producing a genetically engineered T cell, the method involving introducing any of the provided polynucleotides into a T cell comprising a genetic disruption at a TGFBR2 locus.

Provided herein are methods of producing a genetically engineered T cell, the method involving introducing, into a T cell, one or more agent(s) capable of inducing a genetic disruption at a target site within an endogenous TGFBR2 locus of the T cell; and introducing the polynucleotide into a T cell comprising a genetic disruption at a TGFBR2 locus, wherein the method produces a modified TGFBR2 locus, said modified TGFBR2 locus comprising a nucleic acid sequence encoding the recombinant receptor or a portion thereof. In some of any embodiments, the nucleic acid sequence encoding a recombinant receptor or a portion thereof is integrated within the endogenous TGFBR2 locus via homology directed repair (HDR).

Provided herein are methods of producing a genetically engineered T cell, the method involving introducing, into a T cell, a polynucleotide comprising a nucleic acid sequence encoding a recombinant receptor or a portion thereof, said T cell having a genetic disruption within a TGFBR2 locus of the T cell, wherein the nucleic acid sequence encoding the recombinant receptor or a portion thereof is integrated within the endogenous TGFBR2 locus via homology directed repair (HDR). In some of any embodiments, the genetic disruption is carried out by introducing, into a T cell, one or more agent(s) capable of inducing a genetic disruption at a target site within an endogenous TGFBR2 locus of the T cell. In some of any embodiments, the method produces a modified TGFBR2 locus, said modified TGFBR2 locus comprising a nucleic acid sequence encoding a recombinant receptor or a portion thereof. In some of any embodiments, the polynucleotide further comprises one or more homology arm(s) linked to the nucleic acid sequence, wherein the one or more homology arm(s) comprise a sequence homologous to one or more region(s) of an open reading frame of a transforming growth factor-beta receptor type-2 (TGFBR2) locus.

In some of any embodiments, the modified TGFBR2 locus does not encode a functional TGFBRII polypeptide, in a cell generated by the method. In some of any embodiments, the modified TGFBR2 locus does not encode a TGFBRII polypeptide or the expression of TGFBRII polypeptide is eliminated, in a cell generated by the method. In some of any embodiments, the modified TGFBR2 locus does not encode a full length TGFBRII polypeptide or encodes a partial TGFBRII polypeptide, in a cell generated by the method. In some of any embodiments, the modified TGFBR2 locus encodes a dominant negative TGFBRII polypeptide, in a cell generated by the method.

In some of any embodiments, the one or more homology arm comprises a 5′ homology arm and a 3′ homology arm. In some of any embodiments, the polynucleotide comprises the structure [5′ homology arm]-[the nucleic acid sequence encoding a recombinant receptor or a portion thereof]-[3′ homology arm]. In some of any embodiments, the 5′ homology arm and the 3′ homology arm independently are from at or about 50 to at or about 2000 nucleotides, from at or about 100 to at or about 1000 nucleotides, from at or about 100 to at or about 750 nucleotides, from at or about 100 to at or about 600 nucleotides, from at or about 100 to at or about 400 nucleotides, from at or about 100 to at or about 300 nucleotides, from at or about 100 to at or about 200 nucleotides, from at or about 200 to at or about 1000 nucleotides, from at or about 200 to at or about 750 nucleotides, from at or about 200 to at or about 600 nucleotides, from at or about 200 to at or about 400 nucleotides, from at or about 200 to at or about 300 nucleotides, from at or about 300 to at or about 1000 nucleotides, from at or about 300 to at or about 750 nucleotides, from at or about 300 to at or about 600 nucleotides, from at or about 300 to at or about 400 nucleotides, from at or about 400 to at or about 1000 nucleotides, from at or about 400 to at or about 750 nucleotides, from at or about 400 to at or about 600 nucleotides, from at or about 600 to at or about 1000 nucleotides, from at or about 600 to at or about 750 nucleotides or from at or about 750 to at or about 1000 nucleotides in length. In some of any embodiments, the 5′ homology arm and the 3′ homology arm independently are at or about 200, 300, 400, 500, 600, 700 or 800 nucleotides in length, or any value between any of the foregoing. In some of any embodiments, the 5′ homology arm and the 3′ homology arm independently are greater than at or about 300 nucleotides in length. In some of any embodiments, the 5′ homology arm and the 3′ homology arm independently are at or about 400, 500 or 600 nucleotides in length, or any value between any of the foregoing.

In some of any embodiments, the 5′ homology arm comprises the sequence set forth in SEQ ID NOS: 69-71 or a sequence that exhibits at least 85%, 86%, 87%, 88%, 89%, 90%, 91%, 92%, 93%, 94%, 95%, 96%, 97%, 98%, 99% or more sequence identity to SEQ ID NOS: 69-71 or a partial sequence thereof. In some of any embodiments, the 3′ homology arm comprises the sequence set forth in SEQ ID NO:72, or a sequence that exhibits at least 85%, 86%, 87%, 88%, 89%, 90%, 91%, 92%, 93%, 94%, 95%, 96%, 97%, 98%, 99% or more sequence identity to SEQ ID NO:72 or a partial sequence thereof.

In some of any embodiments, the encoded recombinant receptor is a recombinant T cell receptor (TCR). In some of any embodiments, the encoded recombinant receptor comprises a recombinant T cell receptor (TCR). In some of any embodiments, the encoded recombinant receptor is a chimeric antigen receptor (CAR).

In some of any embodiments, the one or more agent(s) capable of inducing a genetic disruption comprises a DNA binding protein or DNA-binding nucleic acid that specifically binds to or hybridizes to the target site, a fusion protein comprising a DNA-targeting protein and a nuclease, or an RNA-guided nuclease. In some of any embodiments, the one or more agent(s) comprises a zinc finger nuclease (ZFN), a TAL-effector nuclease (TALEN), or and a CRISPR-Cas9 combination that specifically binds to, recognizes, or hybridizes to the target site. In some of any embodiments, the each of the one or more agent(s) comprises a guide RNA (gRNA) having a targeting domain that is complementary to the at least one target site. In some of any embodiments, the one or more agent(s) is introduced as a ribonucleoprotein (RNP) complex comprising the gRNA and a Cas9 protein. In some of any embodiments, the RNP is introduced via electroporation, particle gun, calcium phosphate transfection, cell compression or squeezing, such as via electroporation. In some of any embodiments, the concentration of the RNP is from at or about 1 μM to at or about 5 μM. In some of any embodiments, wherein the concentration of the RNP is at or about 2 μM. In some of any embodiments, the gRNA has a targeting domain sequence of GUGGAUGACCUGGCUAACAG (SEQ ID NO:73).

In some of any embodiments, the T cell is a primary T cell derived from a subject. In some of any embodiments, the subject is a human. In some of any embodiments, the T cell is a CD8+ T cell or subtypes thereof. In some of any embodiments, the T cell is a CD4+ T cell or subtypes thereof. In some of any embodiments, the T cell is derived from a multipotent or pluripotent cell. In some of any embodiments, the T cell is derived from a multipotent or pluripotent cell, which is an iPSC.

In some of any embodiments, the polynucleotide is comprised in a viral vector. In some of any embodiments, the viral vector is an AAV vector. In some of any embodiments, the AAV vector is selected from among AAV1, AAV2, AAV3, AAV4, AAV5, AAV6, AAV7 or AAV8 vector. In some of any embodiments, the AAV vector is an AAV2 or AAV6 vector. In some of any embodiments, the viral vector is a retroviral vector. In some of any embodiments, the viral vector is a retroviral vector, which is a lentiviral vector.

In some of any embodiments, the polynucleotide is a linear polynucleotide. In some of any embodiments, the polynucleotide is a linear polynucleotide which is a double-stranded polynucleotide or a single-stranded polynucleotide. In some of any embodiments, the one or more agent(s) and the polynucleotide are introduced simultaneously or sequentially, in any order. In some of any embodiments, the polynucleotide is introduced after the introduction of the one or more agent(s). In some of any embodiments, the polynucleotide is introduced immediately after, or within about 30 seconds, 1 minute, 2 minutes, 3 minutes, 4 minutes, 5 minutes, 6 minutes, 6 minutes, 8 minutes, 9 minutes, 10 minutes, 15 minutes, 20 minutes, 30 minutes, 40 minutes, 50 minutes, 60 minutes, 90 minutes, 2 hours, 3 hours or 4 hours after the introduction of the agent.

In some of any embodiments, prior to the introducing of the one or more agent, the method comprises incubating the cells, in vitro with a stimulatory agent(s) under conditions to stimulate or activate the one or more immune cells. In some of any embodiments, the stimulatory agent(s) comprises and anti-CD3 and anti-CD28 antibodies. In some of any embodiments, the stimulatory agent(s) comprises and anti-CD3 or anti-CD28 antibodies. In some of any embodiments, the stimulatory agent(s) comprises and anti-CD3 and anti-CD28 antibodies, which are anti-CD3/anti-CD28 beads. In some of any embodiments, the stimulatory agent(s) comprises and anti-CD3 or anti-CD28 antibodies, which are anti-CD3/anti-CD28 beads. In some of any embodiments, the stimulatory agent(s) comprises and anti-CD3 and anti-CD28 antibodies, which are anti-CD3/anti-CD28 beads, where the bead to cell ratio is or is about 1:1. In some of any embodiments, the stimulatory agent(s) comprises and anti-CD3 or anti-CD28 antibodies, which are anti-CD3/anti-CD28 beads, where the bead to cell ratio is or is about 1:1.

In some of any embodiments, the method comprises removing the stimulatory agent(s) from the one or more immune cells prior to the introducing with the one or more agents.

In some of any embodiments, the method further comprises incubating the cells prior to, during or subsequent to the introducing of the one or more agents and/or the introducing of the template polynucleotide with one or more recombinant cytokines. In some of any embodiments, the method further comprises incubating the cells prior to, during or subsequent to the introducing of the one or more agents and/or the introducing of the template polynucleotide with one or more recombinant cytokines, where the one or more recombinant cytokines are selected from the group consisting of IL-2, IL-7, and IL-15. In some of any embodiments, the one or more recombinant cytokine is added at a concentration selected from a concentration of IL-2 from at or about 10 U/mL to at or about 200 U/mL. In some of any embodiments, the one or more recombinant cytokine is added at a concentration selected from a concentration of IL-2 from at or about 10 U/mL to at or about 200 U/mL, which is at or about 50 IU/mL to at or about 100 U/mL; IL-7 at a concentration of 0.5 ng/mL to 50 ng/mL. In some of any embodiments, the one or more recombinant cytokine is added at a concentration selected from a concentration of IL-2 from at or about 10 U/mL to at or about 200 U/mL, which is at or about 50 IU/mL to at or about 100 U/mL; IL-7 at a concentration of 0.5 ng/mL to 50 ng/mL, which is at or about 5 ng/mL to at or about 10 ng/mL and/or IL-15 at a concentration of 0.1 ng/mL to 20 ng/mL, such as at or about 0.5 ng/mL to at or about 5 ng/mL. In some of any embodiments, the incubation is carried out subsequent to the introducing of the one or more agents and the introducing of the template polynucleotide for up to or approximately 24 hours, 36 hours, 48 hours, 3, 4, 5, 6, 7, 8, 9, 10, 11, 12, 13, 14, 15, 16, 17, 18, 19, 20 or 21 days. In some of any embodiments, the incubation is carried out subsequent to the introducing of the one or more agents and the introducing of the template polynucleotide for up to or approximately 24 hours, 36 hours, 48 hours, 3, 4, 5, 6, 7, 8, 9, 10, 11, 12, 13, 14, 15, 16, 17, 18, 19, 20 or 21 days, which can be up to or about 7 days.

In some of any embodiments, at least or greater than 35%, 40%, 45%, 50%, 55%, 60%, 65%, 70%, 75%, 80%, or 90% of the cells in a plurality of engineered cells generated by the method comprise a genetic disruption of at least one target site within a TGFBR2 locus. In some of any embodiments, at least or greater than 35%, 40%, 45%, 50%, 55%, 60%, 65%, 70%, 75%, 80%, or 90% of the cells in a plurality of engineered cells generated by the method express the recombinant receptor or antigen-binding fragment thereof.

Provided herein are engineered T cells or a plurality of engineered T cells generated using any of the methods described herein.

Provided herein are compositions comprising the engineered T cell from any of the embodiments described herein.

Provided herein are compositions comprising a plurality of the engineered T cell from any of the embodiments described herein. In some of any embodiments, the composition comprises CD4+ and/or CD8+ T cells. In some of any embodiments, the composition comprises CD4+ and CD8+ T cells and the ratio of CD4+ to CD8+ T cells is from or from about 1:3 to 3:1. In some of any embodiments, the composition comprises CD4+ and CD8+ T cells and the ratio of CD4+ to CD8+ T cells is from or from about 1:3 to 3:1, which can be 1:1. In some of any embodiments, cells expressing the recombinant receptor make up at least 30%, 40%, 50%, 60%, 70%, 80%, 90%, 91%, 92%, 93%, 94%, 95%, 96%, 97%, 98%, 99%, or more of the total cells in the composition or of the total CD4+ or CD8+ cells in the composition.

Provided herein are methods of treatment comprising administering the engineered cell, plurality of engineered cells or composition of any of the embodiments described herein to a subject having a disease or disorder.

Provided herein are uses of the engineered cell, plurality of engineered cells or composition of any of the embodiments described herein for the treatment of a disease or disorder.

Provided herein are uses of the engineered cell, plurality of engineered cells or composition of any of the embodiments described herein in the manufacture of a medicament for treating a disease or disorder.

Provided herein are uses of the engineered cell, plurality of engineered cells or composition of any of the embodiments described herein is for use in the treatment of a disease or disorder.

In some of any embodiments of the method, use or the engineered cell, plurality of engineered cells or composition for use of any of the embodiments described herein, the disease or disorder is a cancer or a tumor.

In some of any embodiments, the cancer or the tumor is a hematologic malignancy, such as a lymphoma, a leukemia, or a plasma cell malignancy. In some of any embodiments, the cancer is a lymphoma and the lymphoma is Burkitt's lymphoma, non-Hodgkin's lymphoma (NHL), Hodgkin's lymphoma, Waldenstrom macroglobulinemia, follicular lymphoma, small non-cleaved cell lymphoma, mucosa-associated lymphatic tissue lymphoma (MALT), marginal zone lymphoma, splenic lymphoma, nodal monocytoid B cell lymphoma, immunoblastic lymphoma, large cell lymphoma, diffuse mixed cell lymphoma, pulmonary B cell angiocentric lymphoma, small lymphocytic lymphoma, primary mediastinal B cell lymphoma, lymphoplasmacytic lymphoma (LPL), or mantle cell lymphoma (MCL). In some of any embodiments, the cancer is a leukemia and the leukemia is chronic lymphocytic leukemia (CLL), plasma cell leukemia or acute lymphocytic leukemia (ALL). In some of any embodiments, the cancer is a plasma cell malignancy and the plasma cell malignancy is multiple myeloma (MM).

In some of any embodiments, the tumor is a solid tumor. In some of any embodiments, the solid tumor is a non-small cell lung cancer (NSCLC) or a head and neck squamous cell carcinoma (HNSCC).

Provided herein are kits that include one or more agent(s) capable of inducing a genetic disruption at a target site within a TGFBR2 locus; and the polynucleotide of any of the embodiments provided herein.

Provided herein are kits that include one or more agent(s) capable of inducing a genetic disruption at a target site within a TGFBR2 locus; and a polynucleotide comprising a nucleic acid sequence encoding recombinant receptor or a portion thereof, wherein the transgene encoding the recombinant receptor or a fragment, such as an antigen-binding fragment, a domain and/or a chain thereof is targeted for integration at or near the target site via homology directed repair (HDR); and instructions for carrying out the method of any of the embodiments provided herein.

### BRIEF DESCRIPTION OF THE DRAWINGS

FIGS. 1A-1D show the anti-tumor activity of the adoptively transferred anti-ROR1 CAR+ T cells, as determined by the change in tumor volume in a tumor-bearing mouse xenograft model NOD.Cg.Prkdc^{scid}IL2rg^{tm1WJl}/SzJ (NSG) injected subcutaneously with H1975 non-small cell lung cancer cells. FIGS. 1A and 1C (group mean; Donor 1 and 2, respectively) and FIGS. 1B and 1D (individual mice; Donor 1 and 2, respectively) show the change in tumor volume for mice administered engineered primary human T cell compositions generated from one of two independent donors (Donor 1, Donor 2), as follows: (1) engineered T cells expressing the anti-ROR1 CAR R12 by lentiviral delivery (LV only), (2) engineered T cells expressing the anti-ROR1 CAR R12 by lentiviral delivery and TGFBR2 knockout (LV+KO), or (3) engineered T cells expressing the anti-ROR1 CAR R12 and DN-TGFBRII by lentiviral delivery (LV+DN), administered at a dose of 1×10⁶ cells (low dose; top panels) or 3×10⁶ cells (high dose; bottom panels); and 3×10⁶ mock treated cells (mock KO) or were untreated (tumor only) as controls.

FIGS. 2A and 2B (Donor 1 and 2, respectively) show the tumor-free survival curve of NSG mice bearing H1975 tumors receiving an adoptive transfer of the engineered cells as described in Example 1.B.

FIGS. 3A (group) and 3B (individual) show the change in tumor volume for the first 14 days after administration of 1×10⁶ engineered T cells to NSG mice bearing H1975 tumors, prior to collection of the tumor, spleen and blood samples, as follows: (1) engineered T cells expressing the anti-ROR1 CAR R12 by lentiviral delivery (LV), (2) engineered T cells expressing the anti-ROR1 CAR R12 by lentiviral delivery and TGFBR2 knockout (LV+KO), or (3) engineered T cells expressing the anti-ROR1 CAR R12 and DN-TGFBRII by lentiviral delivery (LV+DN) at a dose of 1×10⁶ cells, with engineered cells in all groups subject to electroporation.

FIGS. 4A-4B show the frequency of CAR-expressing CD4+(upper panels) and CD8+(lower panels) T cells in the blood (FIG. 4A) or spleen (FIG. 4B) of mice administered cells engineered by various delivery methods as described in Example 2.B. FIGS. 4C-4D show the frequency of CAR-expressing CD4+(upper panel) and CD8+(lower panel) T cells in the tumor (FIG. 4C) and the frequency of CD103+ CAR-expressing CD4+(upper panel) and CD8+(lower panel) T cells in the tumor (FIG. 4D).

FIGS. 5A-5B show the changes in caspase 3/7 activity (FIG. 5A; total green object integrated intensity) and H1975 tumor spheroid size (FIG. 5B; total red object integrated intensity) based on a spheroid killing assay in which isolated tumor-infiltrating lymphocytes (TILs) from the tumor samples or spleen from mice administered engineered T cells engineered using various delivery methods, were incubated with H1975 tumor spheroids at an effector to target ratio of 1:5 in the presence of a low level of TGFβ in serum-containing media. As controls, H1975 tumor spheroid cells were incubated without the engineered cells (tumor only).

FIGS. 6A-6B show the changes in caspase 3/7 activity (FIG. 6A) and H1975 tumor spheroid size (FIG. 6B) based on a spheroid killing assay following incubation with engineered cells expressing an anti-ROR1 CAR R12 or a CAR containing a fully human anti-ROR1 scFv antigen-binding domain, with (fully human KO) a knockout of TGFBR2 or without (fully human WT), with H1975 tumor spheroids at an effector to target ratio of 1:5. As controls, H1975 tumor spheroid cells were incubated without the engineered cells (tumor only). Cells expressing the anti-ROR1 CAR with an scFv antigen-binding domain derived from R12, with a knockout of TGFBR2 (R12 KO) or without (R12 WT), described in Example 1.A above, and cells treated by mock transduction and electroporation without RNPs (mock) or mock transduction with RNPs for TGFBR2 knockout (mock KO) were also assessed as controls.

FIG. 7 depicts surface expression of an exemplary chimeric antigen receptor (CAR) and the side scatter (SSC), as assessed by flow cytometry, in CAR-expressing cells generated by targeting the transgene sequences encoding the exemplary CAR for integration at the endogenous TGFBR2 locus. The transgene sequences also included a) the human elongation factor 1 alpha (EF1α) promoter to drive the expression of the CAR-encoding sequences under the control of a heterologous promoter (EF1α-CAR); or b) sequences encoding a P2A ribosome skip element upstream of the nucleic acid sequences encoding the exemplary CAR (P2A-CAR), to drive expression of the CAR from the endogenous TGFBR2 promoter upon targeted integration in-frame into the TGFBR2 open reading frame (KO/KI). As control, CAR-encoding nucleic acid sequences were incorporated into an exemplary HIV-1 derived lentiviral vector for expression of the CAR from sequences introduced into the T cell by random integration (Lenti). For expression of a dominant negative (DN) form of transforming growth factor beta receptor II (DN-TGFBRII), the lentiviral transduction construct further contained nucleic acid sequences encoding a DN-TGFBRII. The percentage of CAR-expressing cells (CAR+) are indicated.

FIGS. 8A-8C show the anti-ROR1 CAR R12 expression (geometric mean fluorescence by flow cytometry; FIG. 8A), changes in caspase 3/7 activity (FIG. 8B) and H1975 tumor spheroid size (FIG. 8C) based on a spheroid killing assay following incubation with engineered cells expressing an anti-ROR1 CAR R12 engineered using various delivery methods as follows: (1) lentiviral delivery alone (LV), (2) lentiviral delivery with TGFBR2 knockout (LV+KO), (3) lentiviral delivery and expression of dominant negative TGFBRII (LV+DN); or by (4) targeted knock-in at the TGFBR2 locus by HDR (KO/KI).

FIGS. 9A-9C show the changes in anti-ROR1 CAR R12 expression (% CAR+ cells; FIG. 9A) prior to (pre-) or after (post-) a prolonged stimulation assay, changes in caspase 3/7 activity (FIG. 9B) and H1975 tumor spheroid size (FIG. 9C) based on a spheroid killing assay following incubation with engineered cells expressing an anti-ROR1 CAR R12 engineered using various delivery methods and subject to a 7-day prolonged stimulation by beads coated with a recombinant ROR1-Fc fusion protein, incubated at an effector:target (E:T) ratio of 1:5 (top panel) or 1:10 (bottom panel).

FIGS. 10A-10B show the changes in caspase 3/7 activity (FIG. 10A) and H1975 tumor spheroid size (FIG. 10B) based on a spheroid killing assay following incubation with engineered cells expressing an exemplary engineered anti-human papilloma virus 16 (HPV16) T cell receptor (TCR) engineered using various delivery methods as follows: (1) lentiviral delivery alone (TCR), (2) lentiviral delivery with TGFBR2 knockout (TCR+KO), or (3) lentiviral delivery and mock electroporation without RNPs (TCR EP), with (bottom panels) or without (top panels) 10 ng/mL TGFβ in the media. As controls, cells treated by mock transduction (mock), mock transduction and electroporation without RNPs (mock EP) or mock transduction and electroporated with RNPs for a TGFBR2 knockout (mock KO) were also assessed.

FIGS. 11A-11B depict surface expression of an exemplary engineered anti-human papilloma virus 16 (HPV16) T cell receptor (TCR) as stained using an anti-Vbeta2 antibody and the side scatter (SSC), as assessed by flow cytometry, in TCR-expressing cells generated by targeting the transgene sequences encoding the exemplary TCR for integration at the endogenous TGFBR2 locus, under the control of either a) a human elongation factor 1 alpha (EF1α) promoter (EF1α KO/KI) or b) an MND promoter (MND KO/KI). Cells expressing the recombinant TCR by lentiviral delivery with TGFBR2 knockout (TCR LV TGFBR2 KO) or without TGFBR2 knockout (TCR LV) were also assessed. Additional controls included cells subject to mock treatment (mock) and cells with TGFBR2 knockout that were not engineered to express the recombinant TCR (TGFBR2 KO).

FIGS. 12A-12B show the changes in caspase 3/7 activity (FIG. 12A) and H1975 tumor spheroid size (FIG. 12B) based on a spheroid killing assay following incubation with engineered cells expressing an anti-HPV16 TCR engineered using various delivery methods described in Example 6.B, incubated at an effector:target (E:T) ratio of 1:1 (top panels) or 1:5 (bottom panels).

### DETAILED DESCRIPTION

Provided herein are genetically engineered cells such as T cells, having a modified transforming growth factor-beta receptor type 2 (TGFBR2) locus that includes one or more transgene sequence (hereinafter also referred to interchangeably as “donor” sequence, for example, sequences that are exogenous or heterologous to the T cell) encoding a recombinant receptor or a portion thereof. In some aspects, the recombinant receptor or a portion thereof, such as a chimeric antigen receptor (CAR) or a portion thereof, is encoded by transgene sequences that is/are integrated at a TGFBR2 locus in the genome of the cell, resulting in a modified TGFBR2 locus in the genome. In some embodiments, a TGFBRII protein or a portion thereof also is encoded by the modified TGFBR2 locus. In some embodiments, a portion of the TGFBRII is encoded by the modified TGFBR2 can act as a dominant negative form of TGFBRII, for example, by competing with wild-type or unmodified TGFBRII for binding to the transforming growth factor beta (TGFβ) ligand. In some embodiments, expression of the endogenous TGFBR2 gene is knocked out, reduced or eliminated, from the modified TGFBR2 locus in the engineered cell.

Also provided are methods for producing genetically engineered cells containing a modified TGFBR2 locus expressing a recombinant receptor or a portion thereof. The provided embodiments involve specifically targeting transgene sequences encoding the recombinant receptor or a portion thereof to the endogenous TGFBR2 locus. In some contexts, the provided embodiments involve inducing a targeted genetic disruption, e.g., generation of a DNA break, for example, using gene editing methods, and homology-directed repair (HDR) for targeted knock-in of the recombinant receptor-encoding transgene sequences at the endogenous TGFBR2 locus, thereby reducing or eliminating the expression and/or function of the endogenous TGFBR2 gene. Also provided are related cell compositions, nucleic acids and kits for use in generation of the engineered cells provided herein and/or the methods provided herein.

T cell-based therapies, such as adoptive T cell therapies (including those involving the administration of engineered cells expressing recombinant, engineered or chimeric receptors specific for a disease or disorder of interest, such as a chimeric antigen receptor (CAR), a recombinant T cell receptor (TCR) or other recombinant, engineered or chimeric receptors) can be effective in the treatment of cancer and other diseases and disorders. In certain contexts, other approaches for generating engineered cells for adoptive cell therapy may not always be entirely satisfactory. In some aspects, efficacy or potency of the engineered cells can depend on various factors, including T cell exhaustion, immunosuppressive tumor microenvironment (TME), poor cell infiltration into the target, e.g., tumor, and lack of endogenous anti-tumor immune response. In some contexts, optimal activity or outcome can depend on the ability of the administered cells to recognize and bind to a target, e.g., target antigen, to traffic, localize to and successfully enter appropriate sites within the subject, tumors, and environments thereof. In some contexts, optimal activity or outcome can depend on the ability of the administered cells to become activated, expand, to exert various effector functions, including cytotoxic killing and secretion of various factors such as cytokines, to persist, including long-term, to differentiate, transition or engage in reprogramming into certain phenotypic states (such as long-lived memory, less-differentiated, and effector states), to avoid or reduce immunosuppressive conditions in the local microenvironment of a disease, to provide effective and robust recall responses following clearance and re-exposure to target ligand or antigen, and avoid or reduce exhaustion, anergy, peripheral tolerance, terminal differentiation, and/or differentiation into a suppressive state.

In some aspects, the provided embodiments involve inducing a targeted genetic disruption and integration of transgene sequences encoding a recombinant receptor or a portion thereof, by HDR, at the endogenous TGFBR2 locus, thereby altering, reducing or eliminating the expression of TGFBRII from the endogenous TGFBR2 gene. In some aspects, the provided embodiments are based on observations that reduction and/or elimination of expression of TGFBRII, for example by a genetic disruption (e.g., knock-out), and/or a targeted integration (e.g., knock-in) of transgene sequences, such as sequences encoding a recombinant receptor, results in improved activity and/or function, such as anti-tumor activity, cytokine production, expansion and/or persistence, of the engineered cells. In some aspects, the engineered cells can contain a modified TGFBR2 locus, in which the expression of TGFBRII is knocked out, reduced or eliminated, or a modified form of TGFBRII polypeptide is expressed. In some aspects, targeted integration of the transgene sequences can result in expression of a modified form of TGFBRII polypeptide that can compete with or inhibit the function or activity of a wild-type or unmodified TGFBRII expressed in the same cell. In some embodiments, targeted genetic disruption and integration of transgene sequences by HDR can result in expression of a dominant negative (DN) form of the TGFBRII polypeptide, such as a DN form that includes an extracellular domain and a transmembrane domain but lacks all or a portion of the cytoplasmic domain. In some aspects, the modified TGFBRII polypeptide, such as a DN form of TGFBRII, can compete with wild-type or unmodified TGFBRII for binding to the transforming growth factor beta (TGFβ) ligand.

In some contexts, binding of the ligand transforming growth factor beta (TGFβ) to an endogenous TGFBRII, which is a receptor normally expressed on the surface of immune cells, such as T cells, initiates formation of a receptor complex to initiate cellular signaling. TGFβ-mediated cellular signaling in immune cells, such as CD4+ and CD8+ T cells, can result in suppression of CD8+ T cells and induction of regulatory T cell (Treg) phenotypes in CD4+ cells. In some aspects, TGFβ in the TME can affect T cell proliferation, inhibit the maturation of T helper cells and/or reduce T cell effector function. In some aspects, TGFβ can repress the expression of genes involved in cytotoxicity in T cells, such as perforin, granzyme A, granzyme B, IFNγ and Fas ligand. In some aspects, TGFβ can induce the development of Treg cells that can result in immunosuppression. In some aspects, reduction or downregulation of TGFβ mediated cellular signaling, e.g., by knock-out of expression of a receptor for TGFβ such as TGFBRII, or expression of a dominant-negative form of TGFBRII, can result in overcoming suppressive effects of TGFβ signaling in cells (see, e.g., Yang et al., Trends Immunol. (2010) 31(6): 220-227; Oh et al., J Immunol. (2013) 191(8): 3973-3979; Principe et al., Cancer Res. (2016) 76(9): 2525-2539).

In some aspects, the provided embodiments offer an advantage that allows engineered cells administered for adoptive therapy to alleviate or overcome immunosuppressive effects of TGFβ in the tumor microenvironment (TME). In some cases, the TME contains or produces factors or conditions, such as TGFβ, that can mediate immunosuppressive signals to suppress the activity, function, proliferation, survival and/or persistence of T cells administered for T cell therapy. In some embodiments, reduction or elimination of expression of TGFBR2 in the engineered cell permit the engineered cells to alleviate or overcome the immnosuppressive effects, such as immunosuppressive effects of TGFβ-mediated signaling, and promote the function, activity, proliferation, survival and/or persistence of T cells.

In particular embodiments, the provided cells, compositions, nucleic acids, kits and methods can result in improved cell therapies, particularly for cell therapies that target or are specific for an antigen in a tumor microenvironment. In some cases, the provided cells, compositions and methods can result in reduced expression of TGFβ receptor and/or lead to production of a dominant-negative TGFβR (DN TGFβR) that can resist the inhibitory effects of TGFβ, resulting in T cells with longer survival and/or improved function.

In some contexts, the provided methods can be used in connection with solid tumor targets or other disease microenvironments where TGFβ immunosuppressive activity may otherwise impair or reduce the function, survival or activity of a T cell therapy. Moreover, the provided cells, compositions, nucleic acids, kits and methods also offer advantages in controlling and regulating expression of the recombinant receptor, e.g. CAR, on cells of the cell therapy.

In some contexts, the recombinant receptors encoded from the modified TGFBR2 locus in engineered cells provided herein can be encoded under the control of endogenous regulatory elements of the genomic TGFBR2 locus or exogenous regulatory elements. In some aspects, the provided embodiments allow the recombinant receptor to be expressed under the control of the endogenous TGFBR2 regulatory elements or control elements, e.g., cis regulatory elements, such as the promoter, or the 5′ and/or 3′ untranslated regions (UTRs) of the endogenous TGFBR2 locus. In some aspects, such embodiments allow the recombinant receptor, e.g., CAR, or a portion thereof, to be expressed and/or the expression is regulated at a similar level to the endogenous TGFBRII, for example at the nucleic acid level and/or at the protein level.

In some aspects, the provided embodiments allow the recombinant receptor to be expressed under the control of exogenous or heterologous regulatory or control elements, which, in some aspects, provides a more controllable level of expression. In some aspects, the provided embodiments allow targeted and controlled expression of the recombinant receptor in various cell types, including cells in which the endogenous promoter at the endogenous TGFBR2 locus, may not be active.

In some contexts, optimal efficacy of engineered cells can depend on the ability of the administered cells to express the recombinant receptor, including with uniform, homogenous and/or consistent expression of the receptors among cells, such as a population of immune cells and/or cells in a therapeutic cell composition, and for the recombinant receptor to recognize and bind to a target, e.g., target antigen, within the subject, tumors, and environments thereof. In some cases, available methods for introducing a recombinant receptor, such as a CAR, into a cell is by random integration of sequences encoding the recombinant receptor. In certain respects, such methods are not entirely satisfactory. In some aspects, random integration can result in possible insertional mutagenesis and/or genetic disruption of one more random genetic loci in the cell, including those that may be important for cell function and activity. In some cases, semi-random or random integration of a transgene encoding the receptor into the genome of the cell may, in some cases, result in adverse and/or unwanted effects due to integration of the nucleic acid sequence into an undesired location in the genome, e.g., into an essential gene or a gene critical in regulating the activity of the cell.

In some cases, random integration may result in variable integration of the sequences encoding the recombinant or chimeric receptor, which can result in inconsistent expression, variable copy number of the nucleic acids, and/or variability of receptor expression within cells of the cell composition, such as a therapeutic cell composition. In some cases, random integration of a nucleic acid sequence encoding the receptor can result in variegated, heterogeneous, non-uniform and/or suboptimal expression or antigen binding, oncogenic transformation and transcriptional silencing of the nucleic acid sequence, depending on the site of integration and/or nucleic acid sequence copy number. In some aspects, heterogeneous and non-uniform expression in a cell population can lead to inconsistencies or instability of expression and/or antigen binding by the recombinant or chimeric receptor, unpredictability of the function or reduction in function of the engineered cells and/or a non-uniform drug product, thereby reducing the efficacy of the engineered cells. In some aspects, use of particular random integration vectors, such as certain lentiviral vectors, requires confirmation that the engineered cells do not contain replication competent virus. Improved strategies are needed to achieve consistent expression levels and function of the recombinant or chimeric receptors while minimizing random integration of nucleic acids and/or heterogeneous expression in a population.

In some contexts, the provided embodiments relate to engineering a cell to have nucleic acids encoding a recombinant receptor to be integrated into the endogenous TGFBR2 locus of a cell, e.g., T cell, by homology-directed repair (HDR). In some aspects, HDR can mediate the site specific integration of transgene sequences (such as transgene sequences encoding a recombinant receptor or a chimeric receptor or a portion, a chain or a fragment thereof), at or near a target site for genetic disruption, such as an endogenous TGFBR2 locus. In some embodiments, the presence of a genetic disruption (for example, at a target site at the endogenous TGFBR2 locus) and a template polynucleotide containing one or more homology arms (e.g., containing nucleic acid sequences that are homologous to sequences surrounding the genetic disruption) can induce or direct HDR, with homologous sequences acting as a template for DNA repair. Based on homology between the endogenous gene sequence surrounding the genetic disruption and the homology arms included in the template polynucleotide, cellular DNA repair machinery can use the template polynucleotide to repair the DNA break and resynthesize genetic information at the site of the genetic disruption, thereby effectively inserting or integrating the sequences between the homology arms (such as transgene sequences encoding a recombinant receptor or a portion thereof) at or near the target site of the genetic disruption. The provided embodiments can generate cells containing a modified TGFBR2 locus encoding a recombinant receptor or a portion thereof, where transgene sequences encoding a recombinant receptor or a portion thereof is integrated into the endogenous TGFBR2 locus by HDR.

In some aspects, the provided embodiments offer advantages in producing engineered cells with improved and/or more efficient targeting of the nucleic acids encoding the recombinant receptor into the cell, which, at the same time also results in a reduction and/or elimination of expression of TGFBR2 and can result in improved activity and/or function of the engineered cell, or in some cases expression of a dominant negative form of TGFBRII. In some cases, the provided embodiments minimize possible semi-random or random integration and/or heterogeneous or variegated expression, and result in improved, uniform, homogeneous, consistent or stable expression of the recombinant receptor or having reduced, low or no possibility of insertional mutagenesis. In some aspects, compared to other methods of producing genetically engineered immune cells expressing a recombinant or chimeric receptor, e.g., TCR or CAR, the provided embodiments allow for a more stable, more physiological, more controllable or more uniform, consistent or homogeneous expression of the recombinant or chimeric receptor. In some cases, the methods result in the generation of more consistent and more predictable drug product, e.g. cell composition containing the engineered cells, which can result in a safer therapy for treated patients. In some aspects, the provided embodiments also allow predictable and consistent integration at a single gene locus or a multiple gene loci of interest. In some embodiments, the provided embodiments can also result in generating a cell population with consistent copy number (typically, 1 or 2) of the nucleic acids that are integrated in the cells of the population, which, in some aspects, provide consistency in recombinant receptor expression and expression of the endogenous receptor genes within a cell population. In some cases, the provided embodiments do not involve the use of a viral vector for integration and thus can reduce the need for confirmation that the engineered cells do not contain replication competent virus, thereby improving the safety of the cell composition.

Also provided are methods for engineering, preparing, and producing the engineered cells, and kits and devices for generating or producing the engineered cells. Also provided are cells and cell compositions generated by the methods. Provided are polynucleotides, e.g., viral vectors, that contain a nucleic acid sequence encoding a recombinant receptor or a portion thereof, and methods for introducing such polynucleotides into the cells, such as by transduction or by physical delivery, such as electroporation. Also provided are compositions containing the engineered cells, and methods, kits, and devices for administering the cells and compositions to subjects, such as for adoptive cell therapy. In some aspects, the cells are isolated from a subject, engineered, and administered to the same subject. In other aspects, they are isolated from one subject, engineered, and administered to another subject. In some embodiments, the provided polynucleotides, nucleotide sequences, nucleic acid sequences, transgenes, and/or vectors, when delivered into immune cells, result in the expression of recombinant or chimeric receptors, e.g., TCRs or CARs, that can modulate T cell activity, and, in some cases, can modulate T cell differentiation or homeostasis. The resulting genetically engineered cells or cell compositions can be used in adoptive cell therapy methods.

All publications, including patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### I. METHOD FOR GENERATING CELLS EXPRESSING A RECOMBINANT RECEPTOR BY HOMOLOGY-DIRECTED REPAIR

Provided herein are methods of generating or producing genetically engineered cells comprising a modified TGFBR2 locus in which the modified TGFBR2 locus includes nucleic acid sequences encoding a recombinant receptor or a chimeric receptor, such as a chimeric antigen receptor (CAR) or a T cell receptor (TCR). In some aspects, the modified TGFBR2 locus in the genetically engineered cell comprises a transgene sequence encoding a recombinant receptor or a portion thereof, integrated into an endogenous TGFBR2 locus (for example, such that the locus is modified). In some embodiments, the methods involve inducing a targeted genetic disruption and homology-dependent repair (HDR), using polynucleotides (for example, also called “template polynucleotides”) containing the transgene encoding a recombinant receptor or a portion thereof, thereby targeting integration of the transgene at the TGFBR2 locus. Also provided are cells and cell compositions generated by the methods, and polynucleotides, e.g., template polynucleotides, and kits for use in the methods.

In some aspects, the provided embodiments employ HDR for targeted integration of the transgene sequences into the TGFBR2 locus. In some cases, the methods involve introducing one or more targeted genetic disruption(s), e.g., DNA break, at the endogenous TGFBR2 locus by gene editing techniques, combined with targeted integration of transgene sequences encoding a recombinant receptor or a portion thereof by HDR. In some aspects, the one or more targeted genetic disruption(s) is carried out by introduction of one or more agent(s) capable of introducing the genetic disruption(s). In some embodiments, the HDR step entails a disruption or a break, e.g., a double-stranded break, in the DNA at the target genomic location. In some embodiments, the DNA break is induced by employing gene editing methods, e.g., targeted nucleases. In some embodiments, the methods generate an engineered cell that is knocked-out for expression of TGFBR2.

In some aspects, the provided methods involve introducing one or more agent(s) capable of inducing a genetic disruption of at a target site within a TGFBR2 locus into a T cell; and introducing into the T cell a polynucleotide, e.g., a template polynucleotide, comprising a transgene and one or more homology arms. In some aspects, the transgene contains a sequence of nucleotides encoding a recombinant receptor or a portion thereof. In some embodiments, the nucleic acid sequence, such as the transgene, is targeted for integration within the TGFBR2 locus via homology directed repair (HDR). In some aspects, the provided methods involve introducing a polynucleotide comprising a transgene sequence encoding a recombinant receptor or a portion thereof comprising into a T cell having a genetic disruption of within a TGFBR2 locus, wherein the genetic disruption has been induced by one or more agents capable of inducing a genetic disruption of one or more target site within the TGFBR2 locus, and wherein the nucleic acid sequence, such as the transgene, is targeted for integration within the TGFBR2 locus via HDR. In some embodiments, also provided are compositions containing a population of cells that have been engineered to express a recombinant receptor, e.g., a TCR or a CAR, such that the cell population that exhibits more improved, uniform, homogeneous and/or stable expression and/or antigen binding by the recombinant receptor, including genetically engineered immune cells produced by any of the provided methods.

In some aspects, the embodiments involve generating a targeted genomic disruption, such as a targeted DNA break, using gene editing methods and/or targeted nucleases, followed by HDR based on one or more template polynucleotide(s), e.g., template polynucleotide(s) that contains homology sequences that are homologous to sequences at the endogenous TGFBR2 locus linked to transgene sequences encoding recombinant receptor or a portion thereof and optionally nucleic acid sequences encoding other molecules, to specifically target and integrate the transgene sequences at or near the DNA break. Thus, in some aspects, the methods involve a step of inducing a targeted genetic disruption (e.g., via gene editing) and introducing a polynucleotide, e.g., a template polynucleotide comprising transgene sequences, into the cell (e.g., via HDR).

In some embodiments, the targeted genetic disruption and targeted integration of the transgene sequences by HDR occurs at one or more target site(s) at the endogenous TGFBR2 locus. In some aspects, the targeted integration occurs within the open reading frame sequence of the endogenous TGFBR2 locus. In some aspects, targeted integration of the transgene sequences results in a knock-out of the endogenous TGFBR2 gene, e.g., such that the expression of the endogenous TGFBR2 gene is eliminated. In some aspects, targeted integration of the transgene results in expression of a dominant negative (DN) form of the TGFBRII polypeptide. In some aspects, a dominant negative (DN) form (also called an antimorphic mutation) is an altered gene product that acts antagonistically to the wild-type gene product expressed in the same cell. In some aspects, a DN form result in an altered molecular function, optionally inhibiting, counteracting, competing with and/or inactivating the normal function of the gene product, and are characterized by a dominant or semi-dominant phenotype. For example, in some embodiments, a DN form can still interact with the same factors or molecules as the wild-type gene product, but can block some aspect of the function of the wild-type gene product when expressed in the same cell. In some aspects, the transgene sequence has been integrated into the TGFBR2 locus, e.g., by homology-directed repair (HDR) within an exon of an open reading frame or a partial sequence thereof of the endogenous TGFBR2 locus, such that the sequences encoding the recombinant receptor or a portion thereof is in-frame with the sequence of the exon. In some aspects, a portion of the endogenous TGFBR2 locus, such as the portion upstream of the integrated transgene sequences, and the recombinant receptor or portion thereof are expressed in the modified TGFBR2 locus, optionally separated by a multicistronic element. In some aspects, the expressed portion of the endogenous TGFBR2 locus encodes a DN form of TGFBRII.

In some embodiments, a polynucleotide, e.g., template polynucleotide, is introduced into the engineered cell, prior to, simultaneously with, or subsequent to introduction of one or more agent(s) capable of inducing one or more targeted genetic disruption. In the presence of one or more targeted genetic disruption, e.g., DNA break, the template polynucleotide can be used as a DNA repair template, to effectively copy and/or integrate the transgene, at or near the site of the targeted genetic disruption by HDR, based on homology between the endogenous gene sequence surrounding the genetic disruption and the one or more homology arms, such as the 5′ and/or 3′ homology arms, included in the template polynucleotide.

In some aspects, the two steps can be performed sequentially. In some embodiments, the gene editing and HDR steps are performed simultaneously and/or in one experimental reaction. In some embodiments, the gene editing and HDR steps are performed consecutively or sequentially, in one or consecutive experimental reaction(s). In some embodiments, the gene editing and HDR steps are performed in separate experimental reactions, simultaneously or at different times.

The immune cells can include a population of cells containing T cells. Such cells can be cells that have been obtained from a subject, such as obtained from a peripheral blood mononuclear cells (PBMC) sample, an unfractionated T cell sample, a lymphocyte sample, a white blood cell sample, an apheresis product, or a leukapheresis product. In some embodiments, the immune cells, such as the T cells are primary cells, such as primary T cells. In some embodiments, T cells can be separated or selected to enrich T cells in the population using positive or negative selection and enrichment methods. In some embodiments, the population contains CD4+, CD8+ or CD4+ and CD8+ T cells. In some embodiments, the step of introducing the polynucleotide (e.g., template polynucleotide) and the step of introducing the agent (e.g. Cas9/gRNA RNP) can occur simultaneously or sequentially in any order. In some embodiments, the polynucleotide is introduced simultaneously with the introduction of the one or more agents capable of inducing a genetic disruption (e.g. Cas9/gRNA RNP). In particular embodiments, the polynucleotide template is introduced into the immune cells after inducing the genetic disruption by the step of introducing the agent(s) (e.g. Cas9/gRNA RNP). In some embodiments, prior to, during and/or subsequent to introduction of the polynucleotide template and one or more agents (e.g. Cas9/gRNA RNP), the cells are cultured or incubated under conditions to stimulate expansion and/or proliferation of cells.

In particular embodiments of the provided methods, the introduction of the template polynucleotide is performed after the introduction of the one or more agent capable of inducing a genetic disruption. Any method for introducing the one or more agent(s) can be employed as described, depending on the particular agent(s) used for inducing the genetic disruption. In some aspects, the disruption is carried out by gene editing, such as using an RNA-guided nuclease such as a clustered regularly interspersed short palindromic nucleic acid (CRISPR)-Cas system, such as CRISPR-Cas9 system, specific for the TGFBR2 locus being disrupted. In some aspects, the disruption is carried out using a CRISPR-Cas9 system specific for the TGFBR2 locus. In some embodiments, an agent containing a Cas9 and a guide RNA (gRNA) containing a targeting domain, which targets a region of the TGFBR2 locus, is introduced into the cell. In some embodiments, the agent is or comprises a ribonucleoprotein (RNP) complex of Cas9 and gRNA containing the TGFBR2-targeted targeting domain (Cas9/gRNA RNP). In some embodiment, the introduction includes contacting the agent or portion thereof with the cells, in vitro, which can include cultivating or incubating the cell and agent for up to 24, 36 or 48 hours or 3, 4, 5, 6, 7, or 8 days. In some embodiments, the introduction further can include effecting delivery of the agent into the cells. In various embodiments, the methods, compositions and cells according to the present disclosure utilize direct delivery of ribonucleoprotein (RNP) complexes of Cas9 and gRNA to cells, for example by electroporation. In some embodiments, the RNP complexes include a gRNA that has been modified to include a 3′ poly-A tail and a 5′ Anti-Reverse Cap Analog (ARCA) cap. In some cases, electroporation of the cells to be modified includes cold-shocking the cells, e.g. at 32° C. following electroporation of the cells and prior to plating.

In such aspects of the provided methods, the polynucleotide, e.g., template polynucleotide, is introduced into the cells after introduction with the one or more agent(s), such as Cas9/gRNA RNP, e.g. that has been introduced via electroporation. In some embodiments, the polynucleotide, e.g., template polynucleotide, is introduced immediately after the introduction of the one or more agents capable of inducing a genetic disruption. In some embodiments, the polynucleotide, e.g., template polynucleotide, is introduced into the cells within at or about 30 seconds, within at or about 1 minute, within at or about 2 minutes, within at or about 3 minutes, within at or about 4 minutes, within at or about 5 minutes, within at or about 6 minutes, within at or about 6 minutes, within at or about 8 minutes, within at or about 9 minutes, within at or about 10 minutes, within at or about 15 minutes, within at or about 20 minutes, within at or about 30 minutes, within at or about 40 minutes, within at or about 50 minutes, within at or about 60 minutes, within at or about 90 minutes, within at or about 2 hours, within at or about 3 hours or within at or about 4 hours after the introduction of one or more agents capable of inducing a genetic disruption. In some embodiments, the polynucleotide, e.g., template polynucleotide, is introduced into cells at time between at or about 15 minutes and at or about 4 hours after introducing the one or more agent(s), such as between at or about 15 minutes and at or about 3 hours, between at or about 15 minutes and at or about 2 hours, between at or about 15 minutes and at or about 1 hour, between at or about 15 minutes and at or about 30 minutes, between at or about 30 minutes and at or about 4 hours, between at or about 30 minutes and at or about 3 hours, between at or about 30 minutes and at or about 2 hours, between at or about 30 minutes and at or about 1 hour, between at or about 1 hour and at or about 4 hours, between at or about 1 hour and at or about 3 hours, between at or about 1 hour and at or about 2 hours, between at or about 2 hours and at or about 4 hours, between at or about 2 hours and at or about 3 hours or between at or about 3 hours and at or about 4 hours. In some embodiments, the polynucleotide, e.g., template polynucleotide, is introduced into cells at or about 2 hours after the introduction of the one or more agents, such as Cas9/gRNA RNP, e.g. that has been introduced via electroporation.

Any method for introducing the polynucleotide, e.g., template polynucleotide, can be employed as described, depending on the particular methods used for delivery of the polynucleotide, e.g., template polynucleotide, to cells. Exemplary methods include those for transfer of nucleic acids encoding the receptors, including via viral, e.g., retroviral or lentiviral, transduction, transposons, and electroporation. In particular embodiments, viral transduction methods are employed. In some embodiments, template polynucleotides can be transferred or introduced into cells using recombinant infectious virus particles, such as, e.g., vectors derived from simian virus 40 (SV40), adenoviruses, adeno-associated virus (AAV). In some embodiments, recombinant nucleic acids are transferred into T cells using recombinant lentiviral vectors or retroviral vectors, such as gamma-retroviral vectors (see, e.g., Koste et al. (2014) Gene Therapy 2014 Apr. 3. doi: 10.1038/gt.2014.25; Carlens et al. (2000) Exp Hematol 28(10): 1137-46; Alonso-Camino et al. (2013) Mol Ther Nucl Acids 2, e93; Park et al., Trends Biotechnol. 2011 Nov. 29(11): 550-557. In particular embodiments, the viral vector is an AAV such as an AAV2 or an AAV6.

In some embodiments, prior to, during or subsequent to contacting the agent with the cells and/or prior to, during or subsequent to effecting delivery (e.g. electroporation), the provided methods include incubating the cells in the presence of a cytokine, a stimulating agent and/or an agent that is capable of inducing proliferation, stimulation or activation of the immune cells (e.g. T cells). In some embodiments, at least a portion of the incubation is in the presence of a stimulating agent that is or comprises an antibody specific for CD3 an antibody specific for CD28 and/or a cytokine, such as anti-CD3/anti-CD28 beads. In some embodiments, at least a portion of the incubation is in the presence of a cytokine, such as one or more of recombinant IL-2, recombinant IL-7 and/or recombinant IL-15. In some embodiments, the incubation is for up to 8 days before or after the introduction with the one or more agent(s), such as Cas9/gRNA RNP, e.g. via electroporation, and template polynucleotide, such as up to 24 hours, 36 hours or 48 hours or 3, 4, 5, 6, 7 or 8 days.

In some embodiments, the method includes activating or stimulating cells with a stimulating agent (e.g. anti-CD3/anti-CD28 antibodies) prior to introducing the agent, e.g. Cas9/gRNA RNP, and the polynucleotide template. In some embodiments, the incubation in the presence of a stimulating agent (e.g. anti-CD3/anti-CD28) is for 6 hours to 96 hours, such as 24 to 48 hours or 24 to 36 hours prior to the introduction with the one or more agent(s), such as Cas9/gRNA RNP, e.g. via electroporation. In some embodiments, the incubation with the stimulating agents can further include the presence of a cytokine, such as one or more of recombinant IL-2, recombinant IL-7 and/or recombinant IL-15. In some embodiments, the incubation is carried out in the presence of a recombinant cytokine, such as IL-2 (e.g. 1 U/mL to 500 U/mL, such as 10 U/mL to 200 U/mL, for example at least or about 50 U/mL or 100 U/mL), IL-7 (e.g. 0.5 ng/mL to 50 ng/mL, such as 1 ng/mL to 20 ng/mL, for example, at least or about 5 ng/mL or 10 ng/mL) or IL-15 (e.g. 0.1 ng/mL to 50 ng/mL, such as 0.5 ng/mL to 25 ng/mL, for example, at least or about 1 ng/mL or 5 ng/mL). In some embodiments the stimulating agent(s) (e.g. anti-CD3/anti-CD28 antibodies) is washed or removed from the cells prior to introducing or delivering into the cells the agent(s) capable of inducing a genetic disruption Cas9/gRNA RNP and/or the polynucleotide template. In some embodiments, prior to the introducing of the agent(s), the cells are rested, e.g. by removal of any stimulating or activating agent. In some embodiments, prior to introducing the agent(s), the stimulating or activating agent and/or cytokines are not removed.

In some embodiments, subsequent to the introduction of the agent(s), e.g. Cas9/gRNA, and/or the polynucleotide template the cells are incubated, cultivated or cultured in the presence of a recombinant cytokine, such as one or more of recombinant IL-2, recombinant IL-7 and/or recombinant IL-15. In some embodiments, the incubation is carried out in the presence of a recombinant cytokine, such as IL-2 (e.g. 1 U/mL to 500 U/mL, such as 10 U/mL to 200 U/mL, for example at least or about 50 U/mL or 100 U/mL), IL-7 (e.g. 0.5 ng/mL to 50 ng/mL, such as 1 ng/mL to 20 ng/mL, for example, at least or about 5 ng/mL or 10 ng/mL) or IL-15 (e.g. 0.1 ng/mL to 50 ng/mL, such as 0.5 ng/mL to 25 ng/mL, for example, at least or about 1 ng/mL or 5 ng/mL). The cells can be incubated or cultivated under conditions to induce proliferation or expansion of the cells. In some embodiments, the cells can be incubated or cultivated until a threshold number of cells is achieved for harvest, e.g. a therapeutically effective dose.

In some embodiments, the incubation during any portion of the process or all of the process can be at a temperature of 30° C.±2° C. to 39° C.±2° C., such as at least or about at least 30° C.±2° C., 32° C.±2° C., 34° C.±2° C. or 37° C.±2° C. In some embodiments, at least a portion of the incubation is at 30° C.±2° C. and at least a portion of the incubation is at 37° C.±2° C.

In some aspects, the provided embodiments allow the recombinant receptor to be expressed under the control of heterologous or exogenous regulatory or control elements, e.g., a heterologous promoter, such as a constitutive promoter or a regulatable promoter. In some aspects, the provided embodiments allow the recombinant receptor to be expressed under the control of the endogenous TGFBR2 regulatory elements. In some aspects, the provided embodiments allow the nucleic acids encoding the recombinant receptor to be operably linked to the endogenous regulatory or control elements, e.g., cis regulatory elements, such as the promoter, or the 5′ and/or 3′ untranslated regions (UTRs) of the endogenous TGFBR2 locus. Thus, in some aspects, the provided embodiments allow the recombinant receptor, e.g., CAR, to be expressed and/or the expression is regulated at a similar level to the endogenous TGFBR2.

Exemplary methods for carrying out genetic disruption at the endogenous TGFBR2 locus and/or for carrying out HDR for targeted integration of the transgene sequences, such as a portion of a recombinant or chimeric receptor into the TGFBR2 locus are described in the following subsections.

### A. Genetic Disruption

In some embodiments, one or more targeted genetic disruption is induced at the endogenous TGFBR2 locus. In some embodiments, one or more targeted genetic disruption is induced at one or more target sites at or near the endogenous TGFBR2 locus. In some embodiments, the targeted genetic disruption is induced in an exon of the endogenous TGFBR2 locus. In some embodiments, the targeted genetic disruption is induced in an intron of the endogenous TGFBR2 locus. In some aspects, the presence of the one or more targeted genetic disruption and a polynucleotide, e.g., a template polynucleotide that contains transgene sequences encoding a recombinant receptor or a portion thereof, can result in targeted integration of the transgene sequences at or near the one or more genetic disruption (e.g., target site) at the endogenous TGFBR2 locus.

In some embodiments, genetic disruption results in a DNA break, such as a double-strand break (DSB) or a cleavage, or a nick, such as a single-strand break (SSB), at one or more target site in the genome. In some embodiments, at the site of the genetic disruption, e.g., DNA break or nick, action of cellular DNA repair mechanisms can result in knock-out, insertion, missense or frameshift mutation, such as a biallelic frameshift mutation, deletion of all or part of the gene; or, in the presence of a repair template, e.g., a template polynucleotide, can alter the DNA sequence based on the repair template, such as integration or insertion of the nucleic acid sequences, such as a transgene encoding all or a portion of a recombinant receptor, contained in the template. In some embodiments, the genetic disruption can be targeted to one or more exon of a gene or portion thereof. In some embodiments, the genetic disruption can be targeted near a desired site of targeted integration of exogenous sequences, e.g., transgene sequences encoding a recombinant receptor.

In some embodiments, a DNA binding protein or DNA-binding nucleic acid, which specifically binds to or hybridizes to the sequences at a region near one of the at least one target site(s), is used for targeted disruption. In some embodiments, template polynucleotides, e.g., template polynucleotides that include nucleic acid sequences, such as a transgene encoding a recombinant receptor or a portion thereof, and homology sequences, can be introduced for targeted integration by HDR of the recombinant receptor-encoding sequences at or near the site of the genetic disruption, such as described herein, for example, in Section I.B.

In some embodiments, the genetic disruption is carried by introducing one or more agent(s) capable of inducing a genetic disruption. In some embodiments, such agents comprise a DNA binding protein or DNA-binding nucleic acid that specifically binds to or hybridizes to the gene. In some embodiments, the agent comprises various components, such as a fusion protein comprising a DNA-targeting protein and a nuclease or an RNA-guided nuclease. In some embodiments, the agents can target one or more target sites or target locations. In some aspects, a pair of single stranded breaks (e.g., nicks) on each side of the target site can be generated.

In provided embodiments, the term “introducing” encompasses a variety of methods of introducing a nucleic acid and/or a protein, such as DNA, into a cell, either in vitro or in vivo, such methods including transformation, transduction, transfection (e.g. electroporation), and infection. Vectors are useful for introducing DNA encoding molecules into cells. Possible vectors include plasmid vectors and viral vectors. Viral vectors include retroviral vectors, lentiviral vectors, or other vectors such as adenoviral vectors or adeno-associated vectors. Methods, such as electroporation, also can be used to introduce or deliver proteins or ribonucleoprotein (RNP), e.g. containing the Cas9 protein in complex with a targeting gRNA, to cells of interest.

In some embodiments, the genetic disruption occurs at a target site (also known as “target position,” “target DNA sequence” or “target location”), for example, at the endogenous TGFBR2 locus.

In some embodiments, the target site includes a site on a target DNA (e.g., genomic DNA) that is modified by the one or more agent(s) capable of inducing a genetic disruption, e.g., a Cas9 molecule complexed with a gRNA that specifies the target site. For example, the target site can include locations in the DNA at the endogenous TGFBR2 locus, where cleavage or DNA breaks occur. In some aspects, integration of nucleic acid sequences, such as a transgene encoding a recombinant receptor or a portion thereof, by HDR can occur at or near the target site or target sequence. In some embodiments, a target site can be a site between two nucleotides, e.g., adjacent nucleotides, on the DNA into which one or more nucleotides is added. The target site may comprise one or more nucleotides that are altered by a template polynucleotide. In some embodiments, the target site is within a target sequence (e.g., the sequence to which the gRNA binds). In some embodiments, a target site is upstream or downstream of a target sequence.

1. Target Site at an Endogenous TGFBR2 Locus

In some embodiments, the genetic disruption and/or integration of the transgene encoding a recombinant receptor or a portion thereof, via homology-directed repair (HDR), are targeted at an endogenous or genomic locus that encodes the transforming growth factor-beta receptor type II (also known as TGFBRII, TGFBR2, TGFR-2, TGFβ-RII, TGFbeta-RII, TBR-ii, TBRII, AAT3, FAA3, LDS1B, LDS2, LDS2B, MFS2, RIIC or TAAD2).

In humans, TGFBRII is encoded by the transforming growth factor-beta receptor type-2 (TGFBR2) gene. In some embodiments, the genetic disruption, and integration of the transgene encoding a recombinant receptor is targeted at the human TGFBR2 locus, via homology-directed repair (HDR). In some aspects, the genetic disruption is targeted at a target site within the TGFBR2 locus containing an open reading frame encoding TGFBRII, such that targeted integration or insertion of transgene sequences occurs at or near the site of genetic disruption at the TGFBR2 locus. In some aspects, the genetic disruption is targeted at or near an exon of the open reading frame encoding TGFBRII. In some aspects, the genetic disruption is targeted at or near an intron of the open reading frame encoding TGFBRII.

TGFBRII a transmembrane protein that is a member of the serine/threonine protein kinase family and the TGFB receptor subfamily. TGFBRII forms a heterodimeric complex with TGF-beta type I serine/threonine kinase receptor (TGFBRI), a non-promiscuous receptor for the transforming growth factor beta (TGFβ) cytokines TGFβ1, TGFβ2 and TGFβ3 to transduce signals from the cytokines and regulate various physiological and pathological processes, including cell cycle arrest in epithelial and hematopoietic cells, control of mesenchymal cell proliferation and differentiation, wound healing, extracellular matrix production, immunosuppression and carcinogenesis (see, e.g., Yang et al., Trends Immunol. (2010) 31(6): 220-227; Oh et al., J Immunol. (2013) 191(8): 3973-3979; Principe et al., Cancer Res. (2016) 76(9): 2525-2539).

In some aspects, TGFβ is synthesized in a latent form, and is activated to permit formation of a tetrameric receptor complex with TGFβ receptors TGFBRI and TGFBRII. In some aspects, the formation of the receptor complex composed of two TGFBRI and two TGFBRII molecules symmetrically bound to the cytokine dimer results in the phosphorylation and the activation of TGFBRI by the constitutively active TGFBRII. In some cases such as the canonical SMAD-dependent TGFβ-signaling pathways, activated TGFBRI phosphorylates mothers against decapentaplegic homolog 2 (SMAD2), which dissociates from the receptor and interacts with SMAD4. The SMAD2-SMAD4 complex is subsequently translocated to the nucleus where it modulates the transcription of the TGFβ-regulated genes. In some aspects, TGFBRII can also be involved in non-canonical, SMAD-independent TGFβ signaling pathways.

In the context of a tumor or a cancer, TGFβ can promote tumors, e.g., by dysregulation of cyclin-dependent kinase inhibitors, alteration in cytoskeletal architecture, increases in proteases and extracellular matrix formation, decreased immune surveillance and increased angiogenesis.

In some aspects, TGFβ can control immune responses and maintains immune homeostasis through its impact on proliferation, differentiation and survival of multiple immune cell lineages. In some aspects, TGFβ1 is the primary isoform expressed in the immune system, and has a wide-ranging regulatory activity affecting multiple types of immune cells. In some contexts, such as in T cells, binding of TGFβ to TGFBRII can downregulate, inhibit or hinder T cell activation, proliferation and differentiation. TGFβ also can control immune tolerance by virtue of its effect on T cells. For immune cells that can be present in the tumor microenvironment (TME), TGFβ may have an adverse effect on anti-tumor immunity and significantly inhibits tumor immune surveillance. For example, transgenic mice that express a dominant-negative TGFBRII under a T-cell-specific promoter was observed to have spontaneous T-cell differentiation and autoimmune disease (see, e.g., Gorelik et al., Nat. Rev. Immunol. (2002) 2(1):46-53). In some aspects, TGFβ can directly suppresses the cytotoxic activity of cytotoxic T lymphocytes, in some cases via transcriptional repression of genes encoding multiple key molecules, such as perforin, granzymes and cytotoxins. In some aspects, TGFβ regulates the clonal expansion and cytotoxic activity of CD8+ T cells, which can then result in tumor progression or tumor promotion. In some aspects, TGFβ also has a significant impact on CD4+ T-cell differentiation and function, and promotes generation of regulatory T cells (Tregs) and Th17 cells (see, e.g., Principe et al., Cancer Res. (2016) 76(9): 2525-2539). In some aspects, as TGFβ in the context of a tumor promotes tumor progression and can have immunosuppressive activity, reduction, inhibition or deletion of TGFβ signaling components, e.g., TGFβ receptors, can enhance T cell differentiation, function and persistence.

In some aspects, TGFβ is involved in various aspects of carcinogenesis. In some contexts, impaired TGFβ signaling is frequently associated with cancer progression in head and neck squamous cell carcinoma (HNSCC). In some contexts, a reduction or complete loss of TGFBRII is observed in approximately 30% of to 87% of human HNSCC. In some aspects, a loss of Smad4 (22% to 51%) and Smad2 (14% to 38%) expression has been reported in human HNSCC. In some aspects, TGFβ signaling can also be involved in tumor progression by means of loss of epithelial cell adhesion, extracellular matrix remodeling, and enhanced angiogenesis, for example, resulting in promotion of epithelial to mesenchymal transition. In some cases, the level of TGFβ is elevated in HNSCC samples, for example, by 1.5- to 7.5-fold increase compared with normal tissues; and TGFβ levels have been observed to increase by 1.5- to 5.3-fold in 44% of tissue samples with adjacent HNSCC.

Exemplary human TGFBRII precursor polypeptide sequence is set forth in SEQ ID NO:59 (isoform 1; mature polypeptide includes residues 23-567 of SEQ ID NO:59; see Uniprot Accession No. P37173-1; NCBI Reference Sequence: NP_003233.4; mRNA sequence set forth in SEQ ID NO:61, NCBI Reference Sequence: NM_003242.5) or SEQ ID NO:60 (isoform 2; mature polypeptide includes residues 23-592 of SEQ ID NO:60; see Uniprot Accession No. P37173-2; NCBI Reference Sequence: NP_001020018.1; mRNA sequence set forth in SEQ ID NO:62, NCBI Reference Sequence: NM_001024847.2). The two isoforms are produced by alternative splicing.

An exemplary mature TGFBRII contains an extracellular region (including amino acid residues 22-166 of the human TGFBRII precursor sequence (isoform 1) set forth in SEQ ID NO:59, or amino acid residues 22-191 of the human TGFBRII precursor sequence (isoform 2) set forth in SEQ ID NO:60), a transmembrane region (including amino acid residues 167-187 of the human TGFBRII precursor sequence (isoform 1) set forth in SEQ ID NO: 59, or amino acid residues 192-212 of the human TGFBRII precursor sequence (isoform 2) set forth in SEQ ID NO:60), and an intracellular region (including amino acid residues 188-567 of the human TGFBRII precursor sequence (isoform 1) set forth in SEQ ID NO:59, or amino acid residues 213-592 of the human TGFBRII precursor sequence (isoform 2) set forth in SEQ ID NO:60). The TGFBRII contains a serine-threonine/tyrosine-protein kinase catalytic domain, at amino acid residues 244-544 of the human TGFBRII precursor sequence (isoform 1) set forth in SEQ ID NO:59 or at amino acid residues 269-569 of the human TGFBRII precursor sequence (isoform 2) set forth in SEQ ID NO:60. In humans, an exemplary genomic locus encoding TGFBRII, TGFBR2, comprises an open reading frame that contains 7 exons and 6 introns for the transcript variant that encodes isoform 1, or 8 exons and 7 introns for the transcript variant that encodes isoform 2.

An exemplary mRNA transcript of TGFBR2 encoding isoform 1 can span the sequence corresponding to Chromosome 3: 30,606,502-30,694,134 on the forward strand, with reference to human genome version GRCh38 (UCSC Genome Browser on Human December 2013 (GRCh38/hg38) Assembly). Table 1 sets forth the coordinates of the exons and introns of the open reading frames and the untranslated regions of the transcript encoding isoform 1 of an exemplary human TGFBR2 locus.

TABLE 1
Coordinates of exons and introns of exemplary human
TGFBR2 locus, isoform 1 (GRCh38, Chromosome 3, forward strand).

| | Start (GrCh38) | End (GrCh38) | Length |
|---|---|---|---|
| 5′ UTR and Exon 1 | 30,606,502 | 30,606,977 | 476 |
| Intron 1-2 | 30,606,978 | 30,644,746 | 37,769 |
| Exon 2 | 30,644,747 | 30,644,915 | 169 |
| Intron 2-3 | 30,644,916 | 30,650,269 | 5,354 |
| Exon 3 | 30,650,270 | 30,650,460 | 191 |
| Intron 3-4 | 30,650,461 | 30,671,637 | 21,177 |
| Exon 4 | 30,671,638 | 30,672,437 | 800 |
| Intron 4-5 | 30,672,438 | 30,674,104 | 1,667 |
| Exon 5 | 30,674,105 | 30,674,246 | 142 |
| Intron 5-6 | 30,674,247 | 30,688,383 | 14,137 |
| Exon 6 | 30,688,384 | 30,688,511 | 128 |
| Intron 6-7 | 30,688,512 | 30,691,419 | 2,908 |
| Exon 7 and 3′ UTR | 30,691,420 | 30,694,134 | 2,715 |

An exemplary mRNA transcript of TGFBR2 encoding isoform 2 can span the sequence corresponding to Chromosome 3: 30,606,601-30,694,142 on the forward strand, with reference to human genome version GRCh38 (UCSC Genome Browser on Human December 2013 (GRCh38/hg38) Assembly). Table 2 sets forth the coordinates of the exons and introns of the open reading frames and the untranslated regions of the transcript encoding isoform 2 of an exemplary human TGFBR2 locus.

TABLE 2
Coordinates of exons and introns of exemplary human
TGFBR2 locus, isoform 2 (GRCh38, Chromosome 3, forward strand).

| | Start (GrCh38) | End (GrCh38) | Length |
|---|---|---|---|
| 5′ UTR and Exon 1 | 30,606,601 | 30,606,977 | 377 |
| Intron 1-2 | 30,606,978 | 30,623,198 | 16,221 |
| Exon 2 | 30,623,199 | 30,623,273 | 75 |
| Intron 2-3 | 30,623,274 | 30,644,746 | 21,473 |
| Exon 3 | 30,644,747 | 30,644,915 | 169 |
| Intron 3-4 | 30,644,916 | 30,650,269 | 5,354 |
| Exon 4 | 30,650,270 | 30,650,460 | 191 |
| Intron 4-5 | 30,650,461 | 30,671,637 | 21,177 |
| Exon 5 | 30,671,638 | 30,672,437 | 800 |
| Intron 5-6 | 30,672,438 | 30,674,104 | 1,667 |
| Exon 6 | 30,674,105 | 30,674,246 | 142 |
| Intron 6-7 | 30,674,247 | 30,688,383 | 14,137 |
| Exon 7 | 30,688,384 | 30,688,511 | 128 |
| Intron 7-8 | 30,688,512 | 30,691,419 | 2,908 |
| Exon 8 and 3′ UTR | 30,691,420 | 30,694,142 | 2,723 |

In some aspects, the transgene (e.g., exogenous nucleic acid sequences) within the template polynucleotide can be used to guide the location of target sites and/or homology arms. In some aspects, the target site of genetic disruption can be used as a guide to design template polynucleotides and/or homology arms used for HDR. In some embodiments, the genetic disruption can be targeted near a desired site of targeted integration of transgene sequences (for example, encoding a recombinant receptor or a portion thereof). In some aspects, the genetic disruption is targeted such that upon integration of the transgene encoding the recombinant receptor, the expression of the endogenous TGFBR2 gene is reduced or eliminated. In some aspects, the genetic disruption is targeted such that upon integration of the transgene encoding the recombinant receptor, the portion of the endogenous TGFBR2 gene that is expressed encodes a dominant negative form of TGFBRII and/or a non-functional form of TGFBRII.

In certain embodiments, a genetic disruption is targeted at, near, or within a TGFBR2 locus. In particular embodiments, the genetic disruption is targeted at, near, or within an open reading frame of the TGFBR2 locus (such as described in Tables 1 and 2 herein). In certain embodiments, the genetic disruption is targeted at, near, or within an open reading frame that encodes a TCRα constant domain. In some embodiments, the genetic disruption is targeted at, near, or within the TGFBR2 locus (such as described in Tables 1 and 2 herein), or a sequence having at or at least 70%, 75%, 80%, 85%, 90%, 95%, 97%, 98%, 99%, 99.5%, or 99.9% sequence identity to all or a portion, e.g., at or at least 500, 1,000, 1,500, 2,000, 2,500, 3,000, 3,500, or 4,000 contiguous nucleotides, of the TGFBR2 locus (such as described in Tables 1 and 2 herein).

In some aspects, the target site is within an exon of the open reading frame of the endogenous TGFBR2 locus. In some aspects, the target site is within an intron of the open reading frame of the TGFBR2 locus. In some aspects, the target site is within a regulatory or control element, e.g., a promoter, 5′ untranslated region (UTR) or 3′ UTR, of the TGFBR2 locus. In some embodiments, the target site is within the TGFBR2 genomic region sequence described in Tables 1 and 2 herein or any exon or intron of the TGFBR2 genomic region sequence contained therein.

In some embodiments, the target site for a genetic disruption is selected such that after integration of the transgene sequences, the cell is knocked out for, reduced and/or eliminated expression from the endogenous TGFBR2 locus.

In some embodiments, a genetic disruption, e.g., DNA break, is targeted within an exon of the TGFBR2 locus or open reading frame thereof. In certain embodiments, the genetic disruption is within the first exon, second exon, third exon, or forth exon of the TGFBR2 locus or open reading frame thereof. In particular embodiments, the genetic disruption is within the first exon of the TGFBR2 locus or open reading frame thereof. In some embodiments, the genetic disruption is within 500 base pairs (bp) downstream from the 5′ end of the first exon in the TGFBR2 locus or open reading frame thereof. In particular embodiments, the genetic disruption is between the 5′ nucleotide of exon 1 and upstream of the 3′ nucleotide of exon 1. In certain embodiments, the genetic disruption is within 400 bp, 350 bp, 300 bp, 250 bp, 200 bp, 150 bp, 100 bp, or 50 bp downstream from the 5′ end of the first exon in the TGFBR2 locus or open reading frame thereof. In particular embodiments, the genetic disruption is between 1 bp and 400 bp, between 50 and 300 bp, between 100 bp and 200 bp, or between 100 bp and 150 bp downstream from the 5′ end of the first exon in the TGFBR2 locus or open reading frame thereof, each inclusive. In certain embodiments, the genetic disruption is between 100 bp and 150 bp downstream from the 5′ end of the first exon in the TGFBR2 locus or open reading frame thereof, inclusive.

In particular embodiments, the genetic disruption is within the fourth exon of the TGFBR2 locus or the open reading frame of the transcript encoding isoform 1 of an exemplary human TGFBR2 locus (such as described in Table 1 or 2 herein). In some embodiments, the genetic disruption is within 500 base pairs (bp) downstream from the 5′ end of the fourth exon in the TGFBR2 locus or an open reading frame thereof. In particular embodiments, the genetic disruption is between the 5′ nucleotide of exon 4 and upstream of the 3′ nucleotide of exon 4. In certain embodiments, the genetic disruption is within 400 bp, 350 bp, 300 bp, 250 bp, 200 bp, 150 bp, 100 bp, or 50 bp downstream from the 5′ end of the fourth exon in the TGFBR2 locus or open reading frame thereof. In particular embodiments, the genetic disruption is between 1 bp and 400 bp, between 50 and 300 bp, between 100 bp and 200 bp, or between 100 bp and 150 bp downstream from the 5′ end of the fourth exon in the TGFBR2 locus or open reading frame thereof, each inclusive. In certain embodiments, the genetic disruption is between 100 bp and 150 bp downstream from the 5′ end of the fourth exon in the TGFBR2 locus or open reading frame thereof, inclusive.

In particular embodiments, the genetic disruption is targeted within the fifth exon of the TGFBR2 locus or the open reading frame of the transcript encoding isoform 2 of an exemplary human TGFBR2 locus (as described in Table 2 herein). In some embodiments, the genetic disruption is within 500 base pairs (bp) downstream from the 5′ end of the fifth exon in the TGFBR2 locus or an open reading frame thereof. In particular embodiments, the genetic disruption is between the 5′ nucleotide of exon 5 and upstream of the 3′ nucleotide of exon 5. In certain embodiments, the genetic disruption is within 400 bp, 350 bp, 300 bp, 250 bp, 200 bp, 150 bp, 100 bp, or 50 bp downstream from the 5′ end of the fifth exon in the TGFBR2 locus or open reading frame thereof. In particular embodiments, the genetic disruption is between 1 bp and 400 bp, between 50 and 300 bp, between 100 bp and 200 bp, or between 100 bp and 150 bp downstream from the 5′ end of the fifth exon in the TGFBR2 locus or open reading frame thereof, each inclusive. In certain embodiments, the genetic disruption is between 100 bp and 150 bp downstream from the 5′ end of the fifth exon in the TGFBR2 locus or open reading frame thereof, inclusive.

In some aspects, the target site is within an exon, such as exons corresponding to early coding regions. In some embodiments, the target site is within or in close proximity to exons corresponding to early coding region, e.g., exon 1, 2, 3, 4 or 5 of the open reading frame of the endogenous TGFBR2 locus (such as described in Tables 1 and 2 herein), or including sequence immediately following a transcription start site, within exon 1, 2, 3, 4 or 5, or within less than 500, 450, 400, 350, 300, 250, 200, 150, 100 or 50 bp of exon 1, 2, 3, 4 or 5. In some aspects, the target site is at or near exon 1 of the endogenous TGFBR2 locus, e.g., within less than 500, 450, 400, 350, 300, 250, 200, 150, 100 or 50 bp of exon 1. In some embodiments, the target site is at or near exon 2 of the endogenous TGFBR2 locus, or within less than 500, 450, 400, 350, 300, 250, 200, 150, 100 or 50 bp of exon 2. In some aspects, the target site is at or near exon 3 of the endogenous TGFBR2 locus, e.g., within less than 500, 450, 400, 350, 300, 250, 200, 150, 100 or 50 bp of exon 3. In some aspects, the target site is at or near exon 4 of the endogenous TGFBR2 locus, e.g., within less than 500, 450, 400, 350, 300, 250, 200, 150, 100 or 50 bp of exon 4. In some aspects, the target site is at or near exon 5 of the endogenous TGFBR2 locus, e.g., within less than 500, 450, 400, 350, 300, 250, 200, 150, 100 or 50 bp of exon 5. In some aspects, the target site is within a regulatory or control element, e.g., a promoter, of the TGFBR2 locus.

In some aspects, the target site is selected such that targeted integration of the transgene generates an endogenous TGFBR2 locus that encodes dominant negative (DN) form of the TGFBR2. In some aspects, a dominant negative form of the TGFBRII includes a variant of TGFBRII that, when expressed in a cell, can inhibit, reduce or interfere with signal transduction by the TGFβ receptor complex. In some aspects, exemplary dominant negative form of TGFBRII include a truncated TGFBRII, such as a TGFBRII that lacks all or a portion of the cytoplasmic domain. In some embodiments, dominant negative TGFBRII include those described in, e.g., Wieser et al., (1993) Mol. Cell Biol. 13(12): 7239-7247; Brand et al., (1995) JBC 270: 8274-8284; Bottinger et al., (1997) EMBO J 16(10): 2621-2633; Shah et al., (2002) Cancer Res 62:7135-7138; Bollard et al. (2002) Gene Therapy 99(9): 3179-87; and Zhang et al., (2013) Gene Therapy 20: 575-580; and Pang et al. (2013) Cancer Discov. 3(8): 936-951.

In some aspects, exemplary dominant negative form of TGFBRII include a TGFBRII containing a deletion of one or more amino acid residues, optionally one or more contiguous amino acid residues, in the an intracellular region of TGFBRII, e.g., including amino acid residues 188-567 of the human TGFBRII precursor sequence (isoform 1) set forth in SEQ ID NO:59, or amino acid residues 213-592 of the human TGFBRII precursor sequence (isoform 2) set forth in SEQ ID NO:60. In some aspects, an exemplary dominant negative form of TGFBRII includes an amino acid sequence corresponding to residues 22-191 of the amino acid sequence set forth in SEQ ID NO:59, or an amino acid sequence corresponding to residues 22-216 of the amino acid sequence set forth in SEQ ID NO:60, or a sequence that exhibits at least 85%, 86%, 87%, 88%, 89%, 90%, 91%, 92%, 93%, 94%, 95%, 96%, 97%, 98%, or 99% sequence identity thereto or a fragment thereof.

In some aspects, the target site is placed at or near the beginning of the endogenous open reading frame sequences encoding the intracellular regions of the TGFBRII, e.g., amino acid residues 188-567 of the human TGFBRII precursor sequence (isoform 1) set forth in SEQ ID NO:59, or amino acid residues 213-592 of the human TGFBRII precursor sequence (isoform 2) set forth in SEQ ID NO:60. In some embodiments, the target site is located at or near exon 4 of the open reading frame of the transcript encoding isoform 1 of an exemplary human TGFBR2 locus (as described in Table 1 herein), or after, downstream of or 3′ of exon 4 of the open reading frame of the transcript encoding isoform 1 of an exemplary human TGFBR2 locus (as described in Table 1 herein), or at or near exon 5 of the open reading frame of the transcript encoding isoform 2 of an exemplary human TGFBR2 locus (as described in Table 2 herein), or after, downstream of or 3′ of exon 5 of the open reading frame of the transcript encoding isoform 2 of an exemplary human TGFBR2 locus (as described in Table 2 herein). In some embodiments, upon introduction of a genetic disruption at the target site and targeted integration of transgene sequences, e.g., transgene sequences encoding a recombinant receptor or a portion thereof, the encoded polypeptide will include a portion of a TGFBRII polypeptide that is a dominant negative form of the TGFBRII and a recombinant receptor. In some embodiments, upon introduction of a genetic disruption at the target site and targeted integration of transgene sequences, e.g., transgene sequences encoding a recombinant receptor or a portion thereof and containing a ribosome skip element such as a 2A element, the encoded polypeptide will include a portion of a TGFBRII polypeptide that is a dominant negative form of TGFBRII, a ribosome skip sequence, and a recombinant receptor. Thus, upon ribosome skipping and/or self-cleavage, the encoded polypeptide will generate a dominant negative form of TGFBRII and a recombinant receptor.

In certain embodiments, a genetic disruption is targeted at, near, or within a TGFBR2 locus. In particular embodiments, the genetic disruption is targeted at, near, or within an open reading frame of the TGFBR2 locus (such as described in Table 1 or 2 herein). In certain embodiments, the genetic disruption is targeted at, near, or within an open reading frame that encodes a TGFBR2. In some embodiments, the genetic disruption is targeted at, near, or within the TGFBR2 locus (such as described in Table 1 or 2 herein), or a sequence having at or at least 70%, 75%, 80%, 85%, 90%, 95%, 97%, 98%, 99%, 99.5%, or 99.9% sequence identity to all or a portion, e.g., at or at least 500, 1,000, 1,500, 2,000, 2,500, 3,000, 3,500, or 4,000 contiguous nucleotides, of the TGFBR2 locus (such as described in Table 1 or 2 herein).

2. Methods of Genetic Disruption

In some aspects, the methods for generating the genetically engineered cells involve introducing a genetic disruption at one or more target site(s), e.g., one or more target sites at a TGFBR2 locus. Methods for generating a genetic disruption, including those described herein, can involve the use of one or more agent(s) capable of inducing a genetic disruption, such as engineered systems to induce a genetic disruption, a cleavage and/or a double strand break (DSB) or a nick (e.g., a single strand break (SSB)) at a target site or target position in the endogenous or genomic DNA such that repair of the break by an error born process such as non-homologous end joining (NHEJ) or repair by HDR using repair template can result in the insertion of a sequence of interest (e.g., exogenous nucleic acid sequences or transgene encoding a recombinant receptor or a portion thereof) at or near the target site or position. Also provided are one or more agent(s) capable of inducing a genetic disruption, for use in the methods provided herein. In some aspects, the one or more agent(s) can be used in combination with the template nucleotides provided herein, for homology directed repair (HDR) mediated targeted integration of the transgene sequences.

In some embodiments, the one or more agent(s) capable of inducing a genetic disruption comprises a DNA binding protein or DNA-binding nucleic acid that specifically binds to or hybridizes to a particular site or position in the genome, e.g., a target site or target position. In some aspects, the targeted genetic disruption, e.g., DNA break or cleavage, at the endogenous TGFBR2 locus is achieved using a protein or a nucleic acid is coupled to or complexed with a gene editing nuclease, such as in a chimeric or fusion protein. In some embodiments, the one or more agent(s). capable of inducing a genetic disruption comprises an RNA-guided nuclease, or a fusion protein comprising a DNA-targeting protein and a nuclease.

In some embodiments, the agent comprises various components, such as an RNA-guided nuclease, or a fusion protein comprising a DNA-targeting protein and a nuclease. In some embodiments, the targeted genetic disruption is carried out using a DNA-targeting molecule that includes a DNA-binding protein such as one or more zinc finger protein (ZFP) or transcription activator-like effectors (TALEs), fused to a nuclease, such as an endonuclease. In some embodiments, the targeted genetic disruption is carried out using RNA-guided nucleases such as a clustered regularly interspaced short palindromic nucleic acid (CRISPR)-associated nuclease (Cas) system (including Cas and/or Cfp1). In some embodiments, the targeted genetic disruption is carried using agents capable of inducing a genetic disruption, such as sequence-specific or targeted nucleases, including DNA-binding targeted nucleases and gene editing nucleases such as zinc finger nucleases (ZFN) and transcription activator-like effector nucleases (TALENs), and RNA-guided nucleases such as a CRISPR-associated nuclease (Cas) system, specifically designed to be targeted to the at least one target site(s), sequence of a gene or a portion thereof. Exemplary ZFNs, TALEs, and TALENs are described in, e.g., Lloyd et al., Frontiers in Immunology, 4(221): 1-7 (2013).

Zinc finger proteins (ZFPs), transcription activator-like effectors (TALEs), and CRISPR system binding domains can be “engineered” to bind to a predetermined nucleotide sequence, for example via engineering (altering one or more amino acids) of the recognition helix region of a naturally occurring ZFP or TALE protein. Engineered DNA binding proteins (ZFPs or TALEs) are proteins that are non-naturally occurring. Rational criteria for design include application of substitution rules and computerized algorithms for processing information in a database storing information of existing ZFP and/or TALE designs and binding data. See, e.g., U.S. Pat. Nos. 6,140,081; 6,453,242; and 6,534,261; see also WO 98/53058; WO 98/53059; WO 98/53060; WO 02/016536 and WO 03/016496 and U.S. Pub. No. 20110301073.

In some embodiments, the one or more agent(s) specifically targets the at least one target site(s) at or near a TGFBR2 locus. In some embodiments, the agent comprises a ZFN, TALEN or a CRISPR/Cas9 combination that specifically binds to, recognizes, or hybridizes to the target site(s). In some embodiments, the CRISPR/Cas9 system includes an engineered crRNA/tracr RNA (“single guide RNA”) to guide specific cleavage. In some embodiments, the agent comprises nucleases based on the Argonaute system (e.g., from T. thermophilus, known as ‘TtAgo’ (Swarts et al., (2014) Nature 507(7491): 258-261). Targeted cleavage using any of the nuclease systems described herein can be exploited to insert the nucleic acid sequences, e.g., transgene sequences encoding a recombinant receptor or a portion thereof, into a specific target location at an endogenous TGFBR2 locus, using either HDR or NHEJ-mediated processes.

In some embodiments, a “zinc finger DNA binding protein” (or binding domain) is a protein, or a domain within a larger protein, that binds DNA in a sequence-specific manner through one or more zinc fingers, which are regions of amino acid sequence within the binding domain whose structure is stabilized through coordination of a zinc ion. The term zinc finger DNA binding protein is often abbreviated as zinc finger protein or ZFP. Among the ZFPs are artificial ZFP domains targeting specific DNA sequences, typically 9-18 nucleotides long, generated by assembly of individual fingers. ZFPs include those in which a single finger domain is approximately 30 amino acids in length and contains an alpha helix containing two invariant histidine residues coordinated through zinc with two cysteines of a single beta turn, and having two, three, four, five, or six fingers. Generally, sequence-specificity of a ZFP may be altered by making amino acid substitutions at the four helix positions (−1, 2, 3, and 6) on a zinc finger recognition helix. Thus, for example, the ZFP or ZFP-containing molecule is non-naturally occurring, e.g., is engineered to bind to a target site of choice.

In some cases, the DNA-targeting molecule is or comprises a zinc-finger DNA binding domain fused to a DNA cleavage domain to form a zinc-finger nuclease (ZFN). For example, fusion proteins comprise the cleavage domain (or cleavage half-domain) from at least one Type IIS restriction enzyme and one or more zinc finger binding domains, which may or may not be engineered. In some cases, the cleavage domain is from the Type IIS restriction endonuclease FokI, which generally catalyzes double-stranded cleavage of DNA, at 9 nucleotides from its recognition site on one strand and 13 nucleotides from its recognition site on the other. See, e.g., U.S. Pat. Nos. 5,356,802; 5,436,150 and 5,487,994; Li et al. (1992) Proc. Natl. Acad. Sci. USA 89:4275-4279; Li et al. (1993) Proc. Natl. Acad. Sci. USA 90:2764-2768; Kim et al. (1994a) Proc. Natl. Acad. Sci. USA 91:883-887; Kim et al. (1994b) J. Biol. Chem. 269: 978-982. Some gene-specific engineered zinc fingers are available commercially. For example, a platform called CompoZr, for zinc-finger construction is available that provides specifically targeted zinc fingers for thousands of targets. See, e.g., Gaj et al., Trends in Biotechnology, 2013, 31(7), 397-405. In some cases, commercially available zinc fingers are used or are custom designed.

In some embodiments, the one or more target site(s), e.g., within the TGFBR2 locus can be targeted for genetic disruption by engineered ZFNs. Exemplary ZFN that target the endogenous TGFBR2 locus include those encoded by plasmids described in, e.g., NCBI Accession No. NM_029575.3 or NM_031132.

Transcription Activator like Effector (TALE) are proteins from the bacterial species Xanthomonas comprise a plurality of repeated sequences, each repeat comprising di-residues in position 12 and 13 (RVD) that are specific to each nucleotide base of the nucleic acid targeted sequence. Binding domains with similar modular base-per-base nucleic acid binding properties (MBBBD) can also be derived from different bacterial species. The new modular proteins have the advantage of displaying more sequence variability than TAL repeats. In some embodiments, RVDs associated with recognition of the different nucleotides are HD for recognizing C, NG for recognizing T, NI for recognizing A, NN for recognizing G or A, NS for recognizing A, C, G or T, HG for recognizing T, IG for recognizing T, NK for recognizing G, HA for recognizing C, ND for recognizing C, HI for recognizing C, HN for recognizing G, NA for recognizing G, SN for recognizing G or A and YG for recognizing T, TL for recognizing A, VT for recognizing A or G and SW for recognizing A. In some embodiments, critical amino acids 12 and 13 can be mutated towards other amino acid residues in order to modulate their specificity towards nucleotides A, T, C and G and in particular to enhance this specificity.

In some embodiments, a “TALE DNA binding domain” or “TALE” is a polypeptide comprising one or more TALE repeat domains/units. The repeat domains, each comprising a repeat variable diresidue (RVD), are involved in binding of the TALE to its cognate target DNA sequence. A single “repeat unit” (also referred to as a “repeat”) is typically 33-35 amino acids in length and exhibits at least some sequence homology with other TALE repeat sequences within a naturally occurring TALE protein. TALE proteins may be designed to bind to a target site using canonical or non-canonical RVDs within the repeat units. See, e.g., U.S. Pat. Nos. 8,586,526 and 9,458,205.

In some embodiments, a “TALE-nuclease” (TALEN) is a fusion protein comprising a nucleic acid binding domain typically derived from a Transcription Activator Like Effector (TALE) and a nuclease catalytic domain that cleaves a nucleic acid target sequence. The catalytic domain comprises a nuclease domain or a domain having endonuclease activity, like for instance I-TevI, ColE7, NucA and Fok-I. In a particular embodiment, the TALE domain can be fused to a meganuclease like for instance I-CreI and I-OnuI or functional variant thereof. In some embodiments, the TALEN is a monomeric TALEN. A monomeric TALEN is a TALEN that does not require dimerization for specific recognition and cleavage, such as the fusions of engineered TAL repeats with the catalytic domain of I-TevI described in WO2012138927. TALENs have been described and used for gene targeting and gene modifications (see, e.g., Boch et al. (2009) Science 326(5959): 1509-12; Moscou and Bogdanove (2009) Science 326(5959): 1501; Christian et al. (2010) Genetics 186(2): 757-61; Li et al. (2011) Nucleic Acids Res 39(1): 359-72). In some embodiments, one or more sites in the TGFBR2 locus can be targeted for genetic disruption by engineered TALENs.

In some embodiments, a “TtAgo” is a prokaryotic Argonaute protein thought to be involved in gene silencing. TtAgo is derived from the bacteria Thermus thermophilus. See, e.g. Swarts et al., (2014) Nature 507(7491): 258-261, G. Sheng et al., (2013) Proc. Natl. Acad. Sci. U.S.A. 111, 652). A “TtAgo system” is all the components required including e.g. guide DNAs for cleavage by a TtAgo enzyme.

In some embodiments, an engineered zinc finger protein, TALE protein or CRISPR/Cas system is not found in nature and whose production results primarily from an empirical process such as phage display, interaction trap or hybrid selection. See e.g., U.S. Pat. Nos. 5,789,538; 5,925,523; 6,007,988; 6,013,453; 6,200,759; WO 95/19431; WO 96/06166; WO 98/53057; WO 98/54311; WO 00/27878; WO 01/60970; WO 01/88197 and WO 02/099084.

Zinc finger and TALE DNA-binding domains can be engineered to bind to a predetermined nucleotide sequence, for example via engineering (altering one or more amino acids) of the recognition helix region of a naturally occurring zinc finger protein or by engineering of the amino acids involved in DNA binding (the repeat variable diresidue or RVD region). Therefore, engineered zinc finger proteins or TALE proteins are proteins that are non-naturally occurring. Non-limiting examples of methods for engineering zinc finger proteins and TALEs are design and selection. A designed protein is a protein not occurring in nature whose design/composition results principally from rational criteria. Rational criteria for design include application of substitution rules and computerized algorithms for processing information in a database storing information of existing ZFP or TALE designs (canonical and non-canonical RVDs) and binding data. See, for example, U.S. Pat. Nos. 9,458,205; 8,586,526; 6,140,081; 6,453,242; and 6,534,261; see also WO 98/53058; WO 98/53059; WO 98/53060; WO 02/016536 and WO 03/016496.

Various methods and compositions for targeted cleavage of genomic DNA have been described. Such targeted cleavage events can be used, for example, to induce targeted mutagenesis, induce targeted deletions of cellular DNA sequences, and facilitate targeted recombination at a predetermined chromosomal locus. See, e.g., U.S. Pat. Nos. 9,255,250; 9,200,266; 9,045,763; 9,005,973; 9,150,847; 8,956,828; 8,945,868; 8,703,489; 8,586,526; 6,534,261; 6,599,692; 6,503,717; 6,689,558; 7,067,317; 7,262,054; 7,888,121; 7,972,854; 7,914,796; 7,951,925; 8,110,379; 8,409,861; U.S. Patent Publications 20030232410; 20050208489; 20050026157; 20050064474; 20060063231; 20080159996; 201000218264; 20120017290; 20110265198; 20130137104; 20130122591; 20130177983; 20130196373; 20140120622; 20150056705; 20150335708; 20160030477 and 20160024474, the disclosures of which are incorporated by reference in their entireties.

a. CRISPR/Cas9

In some embodiments, the targeted genetic disruption, e.g., DNA break, at the endogenous genes TGFBR2 in humans is carried out using clustered regularly interspaced short palindromic repeats (CRISPR) and CRISPR-associated (Cas) proteins. See Sander and Joung (2014) Nature Biotechnology, 32(4): 347-355.

In general, “CRISPR system” refers collectively to transcripts and other elements involved in the expression of or directing the activity of CRISPR-associated (“Cas”) genes, including sequences encoding a Cas gene, a tracr (trans-activating CRISPR) sequence (e.g. tracr RNA or an active partial tracr RNA), a tracr-mate sequence (encompassing a “direct repeat” and a tracr RNA-processed partial direct repeat in the context of an endogenous CRISPR system), a guide sequence (also referred to as a “spacer” in the context of an endogenous CRISPR system), and/or other sequences and transcripts from a CRISPR locus.

In some aspects, the CRISPR/Cas nuclease or CRISPR/Cas nuclease system includes a non-coding guide RNA (gRNA), which sequence-specifically binds to DNA, and a Cas protein (e.g., Cas9), with nuclease functionality.

Also provided are one or more agents capable of introducing a genetic disruption. Also provided are polynucleotides (e.g., nucleic acid molecules) encoding one or more components of the one or more agent(s) capable of inducing a genetic disruption.

(i) Guide RNA (gRNA)

In some embodiments, the one or more agent(s) capable of inducing a genetic disruption comprises at least one of: a guide RNA (gRNA) having a targeting domain that is complementary with a target site at the TGFBR2 locus or at least one nucleic acid encoding the gRNA.

In some aspects, a “gRNA molecule” is a nucleic acid that promotes the specific targeting or homing of a gRNA molecule/Cas9 molecule complex to a target nucleic acid, such as a locus on the genomic DNA of a cell. gRNA molecules can be unimolecular (having a single RNA molecule), sometimes referred to herein as “chimeric” gRNAs, or modular (comprising more than one, and typically two, separate RNA molecules). In general, a guide sequence, e.g., guide RNA, is any polynucleotide sequences comprising at least a sequence portion that has sufficient complementarity with a target polynucleotide sequence, such as the at the TGFBR2 locus in humans, to hybridize with the target sequence at the target site and direct sequence-specific binding of the CRISPR complex to the target sequence. In some embodiments, in the context of formation of a CRISPR complex, “target sequence” is a sequence to which a guide sequence is designed to have complementarity, where hybridization between the target sequence and a domain, e.g., targeting domain, of the guide RNA promotes the formation of a CRISPR complex. Full complementarity is not necessarily required, provided there is sufficient complementarity to cause hybridization and promote formation of a CRISPR complex. Generally, a guide sequence is selected to reduce the degree of secondary structure within the guide sequence. Secondary structure may be determined by any suitable polynucleotide folding algorithm.

In some embodiments, a guide RNA (gRNA) specific to a target locus of interest (e.g. at the TGFBR2 locus in humans) is used to RNA-guided nucleases, e.g., Cas, to induce a DNA break at the target site or target position. Methods for designing gRNAs and exemplary targeting domains can include those described in, e.g., International PCT Pub. Nos. WO2015/161276, WO2017/193107 and WO2017/093969.

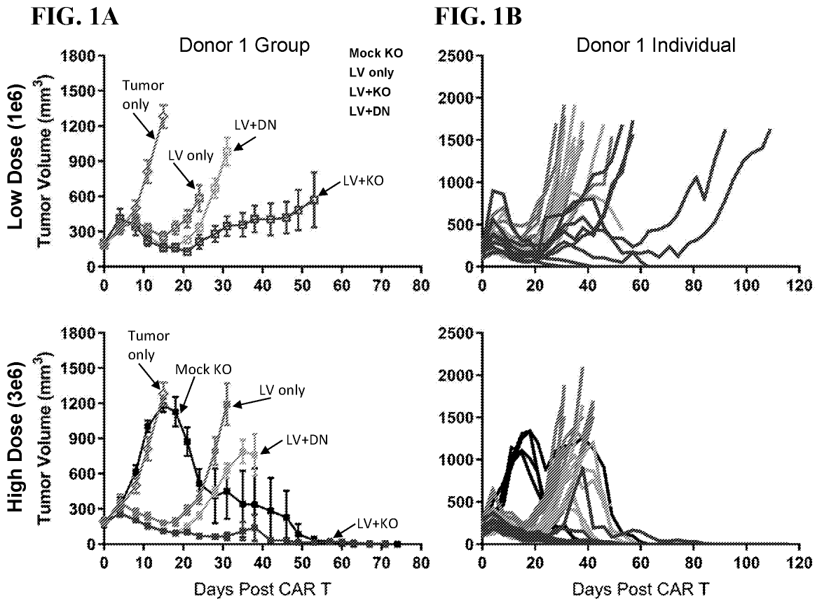

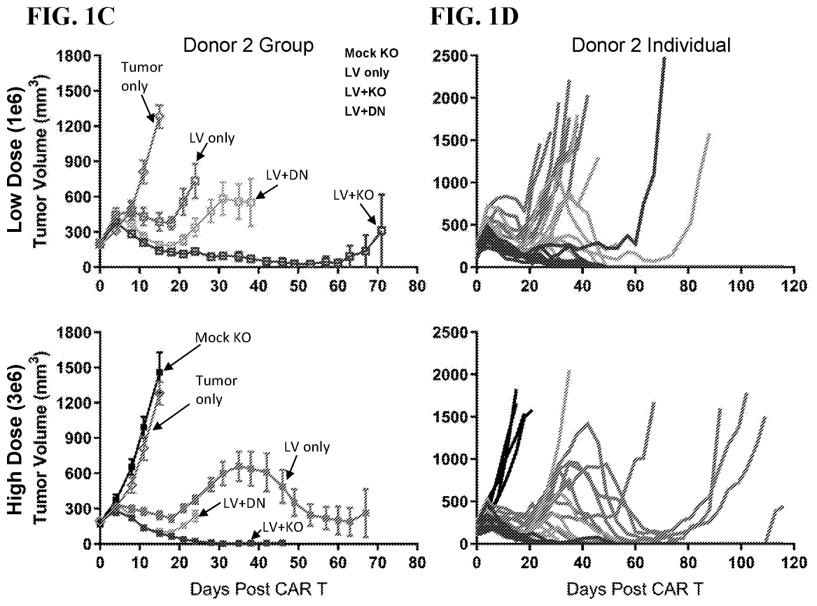

Several exemplary gRNA structures, with domains indicated thereon, are described in WO2015/161276, e.g., in FIGS. 1A-1G therein. While not wishing to be bound by theory, with regard to the three dimensional form, or intra- or inter-strand interactions of an active form of a gRNA, regions of high complementarity are sometimes shown as duplexes in WO2015/161276, e.g., in FIGS. 1A-1G therein and other depictions provided herein.

In some cases, the gRNA is a unimolecular or chimeric gRNA comprising, from 5′ to 3′: a targeting domain which is complementary to a target nucleic acid, such as a sequence from the TGFBR2 gene (coding sequence set forth in SEQ ID NO:74); a first complementarity domain; a linking domain; a second complementarity domain (which is complementary to the first complementarity domain); a proximal domain; and optionally, a tail domain.

In other cases, the gRNA is a modular gRNA comprising first and second strands. In these cases, the first strand preferably includes, from 5′ to 3′: a targeting domain (which is complementary to a target nucleic acid, such as a sequence from the TGFBR2 gene, coding sequence set forth in SEQ ID NO:74 or 76) and a first complementarity domain. The second strand generally includes, from 5′ to 3′: optionally, a 5′ extension domain; a second complementarity domain; a proximal domain; and optionally, a tail domain.

(a) Targeting Domain

The targeting domain comprises a nucleotide sequence that is complementary, e.g., at least 80, 85, 90, 95, 98 or 99% complementary, e.g., fully complementary, to the target sequence on the target nucleic acid. The strand of the target nucleic acid comprising the target sequence is referred to herein as the “complementary strand” of the target nucleic acid. Guidance on the selection of targeting domains can be found, e.g., in Fu Y et al., Nat Biotechnol 2014 (doi: 10.1038/nbt.2808) and Sternberg S H et al., Nature 2014 (doi: 10.1038/nature13011). Examples of the placement of targeting domains include those described in WO2015/161276, e.g., in FIGS. 1A-1G therein.

The targeting domain is part of an RNA molecule and will therefore comprise the base uracil (U), while any DNA encoding the gRNA molecule will comprise the base thymine (T). While not wishing to be bound by theory, In some embodiments, it is believed that the complementarity of the targeting domain with the target sequence contributes to specificity of the interaction of the gRNA molecule/Cas9 molecule complex with a target nucleic acid. It is understood that in a targeting domain and target sequence pair, the uracil bases in the targeting domain will pair with the adenine bases in the target sequence. In some embodiments, the target domain itself comprises in the 5′ to 3′ direction, an optional secondary domain, and a core domain. In some embodiments, the core domain is fully complementary with the target sequence. In some embodiments, the targeting domain is 5 to 50 nucleotides in length. The strand of the target nucleic acid with which the targeting domain is complementary is referred to herein as the complementary strand. Some or all of the nucleotides of the domain can have a modification, e.g., to render it less susceptible to degradation, improve bio-compatibility, etc. By way of non-limiting example, the backbone of the target domain can be modified with a phosphorothioate, or other modification(s). In some cases, a nucleotide of the targeting domain can comprise a 2′ modification, e.g., a 2-acetylation, e.g., a 2′ methylation, or other modification(s).

In various embodiments, the targeting domain is 16-26 nucleotides in length (i.e. it is 16 nucleotides in length, or 17 nucleotides in length, or 18, 19, 20, 21, 22, 23, 24, 25 or 26 nucleotides in length.

(b) Exemplary Targeting Domains

In some embodiments, gRNA sequences that is or comprises a targeting domain sequence targeting the target site in a particular gene, such as the TGFBR2 locus, designed or identified. A genome-wide gRNA database for CRISPR genome editing is publicly available, which contains exemplary single guide RNA (sgRNA) sequences targeting constitutive exons of genes in the human genome or mouse genome (see e.g., genescript.com/gRNA-database.html; see also, Sanjana et al. (2014) Nat. Methods, 11:783-4). In some aspects, the gRNA sequence is or comprises a sequence with minimal off-target binding to a non-target site or position.

In some embodiments, the target sequence (target domain) is at or near the TGFBR2 locus, such as any part of the TGFBR2 coding sequence set forth in SEQ ID NO: 74 or 76. In some embodiments, the target nucleic acid complementary to the targeting domain is located at an early coding region of a gene of interest, such as TGFBR2. Targeting of the early coding region can be used to genetic disruption (i.e., eliminate expression of) the gene of interest. In some embodiments, the early coding region of a gene of interest includes sequence immediately following a start codon (e.g., ATG), or within 500 bp of the start codon (e.g., less than 500, 450, 400, 350, 300, 250, 200, 150, 100, 50 bp, 40 bp, 30 bp, 20 bp, or 10 bp). In particular examples, the target nucleic acid is within 200 bp, 150 bp, 100 bp, 50 bp, 40 bp, 30 bp, 20 bp or 10 bp of the start codon. In some examples, the targeting domain of the gRNA is complementary, e.g., at least 80, 85, 90, 95, 98 or 99% complementary, e.g., fully complementary, to the target sequence on the target nucleic acid, such as the target nucleic acid in the TGFBR2 locus.

In some embodiments, the gRNA can target a site at the TGFBR2 locus near a desired site of targeted integration of transgene sequences, e.g., encoding a recombinant receptor. In some aspects, the gRNA can target a site based on the amount of sequences encoding the TGFBR2 that is desired for expression in the cell expressing the recombinant receptor. In some aspects, the gRNA can target a site such that upon integration of the transgene sequences, e.g., encoding a recombinant receptor, the resulting TGFBR2 locus encodes a dominant negative form of the TGFBRII. In some aspects, the gRNA can target a site within an exon of the open reading frame of the endogenous TGFBR2 locus. In some aspects, the gRNA can target a site within an intron of the open reading frame of the TGFBR2 locus. In some aspects, the gRNA can target a site within a regulatory or control element, e.g., a promoter, of the TGFBR2 locus. In some aspects, the target site at the TGFBR2 locus that is targeted by the gRNA can be any target sites described herein, e.g., in Section I.A.1. In some embodiments, the gRNA can target a site within or in close proximity to exons corresponding to early coding region, e.g., exon 1, 2, 3, 4 or 5 of the open reading frame of the endogenous TGFBR2 locus, or including sequence immediately following a transcription start site, within exon 1, 2, 3, 4 or 5, or within less than 500, 450, 400, 350, 300, 250, 200, 150, 100 or 50 bp of exon 1, 2, 3, 4 or 5. In some embodiments, the gRNA can target a site at or near exon 2 of the endogenous TGFBR2 locus, or within less than 500, 450, 400, 350, 300, 250, 200, 150, 100 or 50 bp of exon 2.

Exemplary target site sequences for disruption of the human at the TGFBR2 locus using Cas9 can include any set forth in SEQ ID NOS: 63-68 and 73. Exemplary gRNAs can include a sequence of ribonucleic acids that can bind to or target or is complementary to or can bind to the complimentary strand sequence of the target site sequences set forth in any of SEQ ID NOS: 74-76, 80, 81, 87-96 and 127-182. Any of the known methods can be used to target and generate a genetic disruption of the endogenous TGFBR2 locus can be used in the embodiments provided herein.

In some embodiments, targeting domains include those for introducing a genetic disruption at the TGFBR2 gene using S. pyogenes Cas9 or using N. meningitidis Cas9. In some embodiments, targeting domains include those for introducing a genetic disruption at the TGFBR2 gene using S. pyogenes Cas9. Any of the targeting domains can be used with a S. pyogenes Cas9 molecule that generates a double stranded break (Cas9 nuclease) or a single-stranded break (Cas9 nickase).

In some embodiments, dual targeting is used to create two nicks on opposite DNA strands by using S. pyogenes Cas9 nickases with two targeting domains that are complementary to opposite DNA strands, e.g., a gRNA comprising any minus strand targeting domain may be paired with any gRNA comprising a plus strand targeting domain. In some embodiments, the two gRNAs are oriented on the DNA such that PAMs face outward and the distance between the 5′ ends of the gRNAs is 0-50 bp. In some embodiments, two gRNAs are used to target two Cas9 nucleases or two Cas9 nickases, for example, using a pair of Cas9 molecule/gRNA molecule complex guided by two different gRNA molecules to cleave the target domain with two single stranded breaks on opposing strands of the target domain. In some embodiments, the two Cas9 nickases can include a molecule having HNH activity, e.g., a Cas9 molecule having the RuvC activity inactivated, e.g., a Cas9 molecule having a mutation at D10, e.g., the D10A mutation, a molecule having RuvC activity, e.g., a Cas9 molecule having the HNH activity inactivated, e.g., a Cas9 molecule having a mutation at H840, e.g., a H840A, or a molecule having RuvC activity, e.g., a Cas9 molecule having the HNH activity inactivated, e.g., a Cas9 molecule having a mutation at N863, e.g., N863A. In some embodiments, each of the two gRNAs are complexed with a D10A Cas9 nickase

(c) The First Complementarity Domain

The first complementarity domain is complementary with the second complementarity domain described herein, and generally has sufficient complementarity to the second complementarity domain to form a duplexed region under at least some physiological conditions. The first complementarity domain is typically 5 to 30 nucleotides in length, and may be 5 to 25 nucleotides in length, 7 to 25 nucleotides in length, 7 to 22 nucleotides in length, 7 to 18 nucleotides in length, or 7 to 15 nucleotides in length. In various embodiments, the first complementary domain is 5, 6, 7, 8, 9, 10, 11, 12, 13, 14, 15, 16, 17, 18, 19, 20, 21, 22, 23, 24, or 25 nucleotides in length. Examples of first complementarity domains include those described in WO2015/161276, e.g., in FIGS. 1A-1G therein.

Typically, the first complementarity domain does not have exact complementarity with the second complementarity domain target. In some embodiments, the first complementarity domain can have 1, 2, 3, 4 or 5 nucleotides that are not complementary with the corresponding nucleotide of the second complementarity domain. In some embodiments, a segment of 1, 2, 3, 4, 5 or 6, (e.g., 3) nucleotides of the first complementarity domain may not pair in the duplex, and may form a non-duplexed or looped-out region. In some instances, an unpaired, or loop-out, region, e.g., a loop-out of 3 nucleotides, is present on the second complementarity domain. This unpaired region optionally begins 1, 2, 3, 4, 5, or 6, e.g., 4, nucleotides from the 5′ end of the second complementarity domain.

The first complementarity domain can include 3 subdomains, which, in the 5′ to 3′ direction are: a 5′ subdomain, a central subdomain, and a 3′ subdomain. In some embodiments, the 5′ subdomain is 4-9, e.g., 4, 5, 6, 7, 8 or 9 nucleotides in length. In some embodiments, the central subdomain is 1, 2, or 3, e.g., 1, nucleotide in length. In some embodiments, the 3′ subdomain is 3 to 25, e.g., 4-22, 4-18, or 4 to 10, or 3, 4, 5, 6, 7, 8, 9, 10, 11, 12, 13, 14, 15, 16, 17, 18, 19, 20, 21, 22, 23, 24, or 25, nucleotides in length.

In some embodiments, the first and second complementarity domains, when duplexed, comprise 11 paired nucleotides, for example, in the gRNA sequence (one paired strand underlined, one bolded):

(SEQ ID NO: 97)
NNNNNNNNNNNNNNNNNNNNGUUUUAGAGCUAGAAAUAGCAAGUUAAAA
UAAGGCUAGUCCGUUAUCAACUUGAAAAAGUGGCACCGAGUCGGUGC.

In some embodiments, the first and second complementarity domains, when duplexed, comprise 15 paired nucleotides, for example in the gRNA sequence (one paired strand underlined, one bolded):

(SEQ ID NO: 98)
NNNNNNNNNNNNNNNNNNNNGUUUUAGAGCUAUGCUGAAAAGCAUAGCA
AGUUAAAAUAAGGCUAGUCCGUUAUCAACUUGAAAAAGUGGCACCGAGU
CGGUGC.

In some embodiments the first and second complementarity domains, when duplexed, comprise 16 paired nucleotides, for example in the gRNA sequence (one paired strand underlined, one bolded):

(SEQ ID NO: 99)
NNNNNNNNNNNNNNNNNNNNGUUUUAGAGCUAUGCUGGAAACAGCAUAG
CAAGUUAAAAUAAGGCUAGUCCGUUAUCAACUUGAAAAAGUGGCACCGA
GUCGGUGC.

In some embodiments the first and second complementarity domains, when duplexed, comprise 21 paired nucleotides, for example in the gRNA sequence (one paired strand underlined, one bolded):

(SEQ ID NO: 100)
NNNNNNNNNNNNNNNNNNNNGUUUUAGAGCUAUGCUGUUUUGGAAACAA
AACAGCAUAGCAAGUUAAAAUAAGGCUAGUCCGUUAUCAACUUGAAAAA
GUGGCACCGAGUCGGUGC.

In some embodiments, nucleotides are exchanged to remove poly-U tracts, for example in the gRNA sequences (exchanged nucleotides underlined):

(SEQ ID NO: 101)
NNNNNNNNNNNNNNNNNNNNGUAUUAGAGCUAGAAAUAGCAAGUUAAUA
UAAGGCUAGUCCGUUAUCAACUUGAAAAAGUGGCACCGAGUCGGUGC;
(SEQ ID NO: 102)
NNNNNNNNNNNNNNNNNNNNGUUUAAGAGCUAGAAAUAGCAAGUUUAAA
UAAGGCUAGUCCGUUAUCAACUUGAAAAAGUGGCACCGAGUCGGUGC;
and
(SEQ ID NO: 103)
NNNNNNNNNNNNNNNNNNNNGUAUUAGAGCUAUGCUGUAUUGGAAACAA
UACAGCAUAGCAAGUUAAUAUAAGGCUAGUCCGUUAUCAACUUGAAAAA
GUGGCACCGAGUCGGUGC.

The first complementarity domain can share homology with, or be derived from, a naturally occurring first complementarity domain. In some embodiments, it has at least 50% homology with a first complementarity domain disclosed herein, e.g., an S. pyogenes, S. aureus, N. meningtidis, or S. thermophilus, first complementarity domain.

It should be noted that one or more, or even all of the nucleotides of the first complementarity domain, can have a modification along the lines discussed herein for the targeting domain.

(d) The Linking Domain

In a unimolecular or chimeric gRNA, the linking domain serves to link the first complementarity domain with the second complementarity domain of a unimolecular gRNA. The linking domain can link the first and second complementarity domains covalently or non-covalently. In some embodiments, the linkage is covalent. In some embodiments, the linking domain covalently couples the first and second complementarity domains, see, e.g., WO2015/161276, e.g., in FIGS. 1B-1E therein. In some embodiments, the linking domain is, or comprises, a covalent bond interposed between the first complementarity domain and the second complementarity domain. Typically the linking domain comprises one or more, e.g., 2, 3, 4, 5, 6, 7, 8, 9, or 10 nucleotides, but in various embodiments the linker can be 20, 30, 40, 50 or even 100 nucleotides in length. Examples of linking domains include those described in WO2015/161276, e.g., in FIGS. 1A-1G therein.

In modular gRNA molecules, the two molecules are associated by virtue of the hybridization of the complementarity domains and a linking domain may not be present. See e.g., WO2015/161276, e.g., in FIG. 1A therein.

A wide variety of linking domains are suitable for use in unimolecular gRNA molecules. Linking domains can consist of a covalent bond, or be as short as one or a few nucleotides, e.g., 1, 2, 3, 4, or 5 nucleotides in length. In some embodiments, a linking domain is 2, 3, 4, 5, 6, 7, 8, 9, 10, 15, 20, or 25 or more nucleotides in length. In some embodiments, a linking domain is 2 to 50, 2 to 40, 2 to 30, 2 to 20, 2 to 10, or 2 to 5 nucleotides in length. In some embodiments, a linking domain shares homology with, or is derived from, a naturally occurring sequence, e.g., the sequence of a tracrRNA that is 5′ to the second complementarity domain. In some embodiments, the linking domain has at least 50% homology with a linking domain disclosed herein.

As discussed herein in connection with the first complementarity domain, some or all of the nucleotides of the linking domain can include a modification.

(e) The 5′ Extension Domain

In some cases, a modular gRNA can comprise additional sequence, 5′ to the second complementarity domain, referred to herein as the 5′ extension domain. In some embodiments, the 5′ extension domain is, 2-10, 2-9, 2-8, 2-7, 2-6, 2-5, or 2-4 nucleotides in length. In some embodiments, the 5′ extension domain is 2, 3, 4, 5, 6, 7, 8, 9, or 10 or more nucleotides in length. In some embodiments, examples of a 5′ extension domain include those described in WO2015/161276, e.g., in FIG. 1A therein.

(f) The Second Complementarity Domain

The second complementarity domain is complementary with the first complementarity domain, and generally has sufficient complementarity to the second complementarity domain to form a duplexed region under at least some physiological conditions. In some cases, e.g., as shown in WO2015/161276, e.g., in FIG. 1A-1B therein, the second complementarity domain can include sequence that lacks complementarity with the first complementarity domain, e.g., sequence that loops out from the duplexed region. Examples of second complementarity domains include those described in WO2015/161276, e.g., in FIGS. 1A-1G therein.

The second complementarity domain may be 5 to 27 nucleotides in length, and in some cases may be longer than the first complementarity region. In some embodiments, the second complementary domain can be 7 to 27 nucleotides in length, 7 to 25 nucleotides in length, 7 to 20 nucleotides in length, or 7 to 17 nucleotides in length. More generally, the complementary domain may be 5, 6, 7, 8, 9, 10, 11, 12, 13, 14, 15, 16, 17, 18, 19, 20, 21, 22, 23, 24, 25, or 26 nucleotides in length.

In some embodiments, the second complementarity domain comprises 3 subdomains, which, in the 5′ to 3′ direction are: a 5′ subdomain, a central subdomain, and a 3′ subdomain. In some embodiments, the 5′ subdomain is 3 to 25, e.g., 4 to 22, 4 to 18, or 4 to 10, or 3, 4, 5, 6, 7, 8, 9, 10, 11, 12, 13, 14, 15, 16, 17, 18, 19, 20, 21, 22, 23, 24, or 25 nucleotides in length. In some embodiments, the central subdomain is 1, 2, 3, 4 or 5, e.g., 3, nucleotides in length. In some embodiments, the 3′ subdomain is 4 to 9, e.g., 4, 5, 6, 7, 8 or 9 nucleotides in length.

In some embodiments, the 5′ subdomain and the 3′ subdomain of the first complementarity domain, are respectively, complementary, e.g., fully complementary, with the 3′ subdomain and the 5′ subdomain of the second complementarity domain.

The second complementarity domain can share homology with or be derived from a naturally occurring second complementarity domain. In some embodiments, it has at least 50% homology with a second complementarity domain disclosed herein, e.g., an S. pyogenes, S. aureus, N. meningtidis, or S. thermophilus, first complementarity domain.

Some or all of the nucleotides of the second complementarity domain can have a modification, e.g., a modification described herein.

(g) The Proximal domain

Examples of proximal domains include those described in WO2015/161276, e.g., in FIGS. 1A-1G therein. In some embodiments, the proximal domain is 5 to 20 nucleotides in length. In some embodiments, the proximal domain can share homology with or be derived from a naturally occurring proximal domain. In some embodiments, it has at least 50% homology with a proximal domain disclosed herein, e.g., an S. pyogenes, S. aureus, N. meningtidis, or S. thermophilus, proximal domain.

Some or all of the nucleotides of the proximal domain can have a modification along the lines described herein.

(h) The Tail Domain

As can be seen by inspection of the tail domains in WO2015/161276, e.g., in FIG. 1A and FIGS. 1B-1F therein, a broad spectrum of tail domains are suitable for use in gRNA molecules. In various embodiments, the tail domain is 0 (absent), 1, 2, 3, 4, 5, 6, 7, 8, 9, or 10 nucleotides in length. In certain embodiments, the tail domain nucleotides are from or share homology with sequence from the 5′ end of a naturally occurring tail domain, see e.g., WO2015/161276, e.g., in FIG. 1D or 1E therein. The tail domain also optionally includes sequences that are complementary to each other and which, under at least some physiological conditions, form a duplexed region. Examples of tail domains include those described in WO2015/161276, e.g., in FIGS. 1A-1G therein.

Tail domains can share homology with or be derived from naturally occurring proximal tail domains. By way of non-limiting example, a given tail domain according to various embodiments of the present disclosure may share at least 50% homology with a naturally occurring tail domain disclosed herein, e.g., an S. pyogenes, S. aureus, N. meningtidis, or S. thermophilus, tail domain.

In certain cases, the tail domain includes nucleotides at the 3′ end that are related to the method of in vitro or in vivo transcription. When a T7 promoter is used for in vitro transcription of the gRNA, these nucleotides may be any nucleotides present before the 3′ end of the DNA template. When a U6 promoter is used for in vivo transcription, these nucleotides may be the sequence UUUUUU. When alternate pol-III promoters are used, these nucleotides may be various numbers or uracil bases or may include alternate bases.

As a non-limiting example, in various embodiments the proximal and tail domain, taken together comprise the following sequences:

(SEQ ID NO: 104)
AAGGCUAGUCCGUUAUCAACUUGAAAAAGUGGCACCGAGUCGGUGCU,
(SEQ ID NO: 105)
AAGGCUAGUCCGUUAUCAACUUGAAAAAGUGGCACCGAGUCGGUGGUGC,
(SEQ ID NO: 106)
AAGGCUAGUCCGUUAUCAACUUGAAAAAGUGGCACCGAGUCGGUGCGGA
UC,
(SEQ ID NO: 107)
AAGGCUAGUCCGUUAUCAACUUGAAAAAGUG,
(SEQ ID NO: 108)
AAGGCUAGUCCGUUAUCA,
or
(SEQ ID NO: 109)
AAGGCUAGUCCG.

In some embodiments, the tail domain comprises the 3′ sequence UUUUUU, e.g., if a U6 promoter is used for transcription. In some embodiments, the tail domain comprises the 3′ sequence UUUU, e.g., if an H1 promoter is used for transcription. In some embodiments, tail domain comprises variable numbers of 3′ Us depending, e.g., on the termination signal of the pol-III promoter used. In some embodiments, the tail domain comprises variable 3′ sequence derived from the DNA template if a T7 promoter is used. In some embodiments, the tail domain comprises variable 3′ sequence derived from the DNA template, e.g., if in vitro transcription is used to generate the RNA molecule. In some embodiments, the tail domain comprises variable 3′ sequence derived from the DNA template, e.g., if a pol-II promoter is used to drive transcription.

In some embodiments a gRNA has the following structure: 5′ [targeting domain]-[first complementarity domain]-[linking domain]-[second complementarity domain]-[proximal domain]-[tail domain]-3′, wherein, the targeting domain comprises a core domain and optionally a secondary domain, and is 10 to 50 nucleotides in length; the first complementarity domain is 5 to 25 nucleotides in length and, In some embodiments has at least 50, 60, 70, 80, 85, 90, 95, 98 or 99% homology with a reference first complementarity domain disclosed herein; the linking domain is 1 to 5 nucleotides in length; the proximal domain is 5 to 20 nucleotides in length and, In some embodiments has at least 50, 60, 70, 80, 85, 90, 95, 98 or 99% homology with a reference proximal domain disclosed herein; and the tail domain is absent or a nucleotide sequence is 1 to 50 nucleotides in length and, In some embodiments has at least 50, 60, 70, 80, 85, 90, 95, 98 or 99% homology with a reference tail domain disclosed herein.

(i) Exemplary Chimeric gRNAs

In some embodiments, a unimolecular, or chimeric, gRNA comprises, preferably from 5′ to 3′: a targeting domain, e.g., comprising 15, 16, 17, 18, 19, 20, 21, 22, 23, 24, 25, or 26 nucleotides (which is complementary to a target nucleic acid); a first complementarity domain; a linking domain; a second complementarity domain (which is complementary to the first complementarity domain); a proximal domain; and a tail domain, wherein, (a) the proximal and tail domain, when taken together, comprise at least 15, 18, 20, 25, 30, 31, 35, 40, 45, 49, 50, or 53 nucleotides; (b) there are at least 15, 18, 20, 25, 30, 31, 35, 40, 45, 49, 50, or 53 nucleotides 3′ to the last nucleotide of the second complementarity domain; or (c) there are at least 16, 19, 21, 26, 31, 32, 36, 41, 46, 50, 51, or 54 nucleotides 3′ to the last nucleotide of the second complementarity domain that is complementary to its corresponding nucleotide of the first complementarity domain.

In some embodiments, the sequence from (a), (b), or (c), has at least 60, 75, 80, 85, 90, 95, or 99% homology with the corresponding sequence of a naturally occurring gRNA, or with a gRNA described herein. In some embodiments, the proximal and tail domain, when taken together, comprise at least 15, 18, 20, 25, 30, 31, 35, 40, 45, 49, 50, or 53 nucleotides. In some embodiments, there are at least 15, 18, 20, 25, 30, 31, 35, 40, 45, 49, 50, or 53 nucleotides 3′ to the last nucleotide of the second complementarity domain. In some embodiments, there are at least 16, 19, 21, 26, 31, 32, 36, 41, 46, 50, 51, or 54 nucleotides 3′ to the last nucleotide of the second complementarity domain that is complementary to its corresponding nucleotide of the first complementarity domain. In some embodiments, the targeting domain comprises, has, or consists of, 16, 17, 18, 19, 20, 21, 22, 23, 24, 25 or 26 nucleotides (e.g., 16, 17, 18, 19, 20, 21, 22, 23, 24, 25 or 26 consecutive nucleotides) having complementarity with the target domain, e.g., the targeting domain is 16, 17, 18, 19, 20, 21, 22, 23, 24, 25 or 26 nucleotides in length.

In some embodiments, the unimolecular, or chimeric, gRNA molecule (comprising a targeting domain, a first complementary domain, a linking domain, a second complementary domain, a proximal domain and, optionally, a tail domain) comprises the following sequence in which the targeting domain is depicted as 20 Ns but could be any sequence and range in length from 16 to 26 nucleotides and in which the gRNA sequence is followed by 6 Us, which serve as a termination signal for the U6 promoter, but which could be either absent or fewer in number: NNNNNNNNNNNNNNNNNNNNGUUUUAGAGCUAGAAAUAGCAAGUUAAAAUAAGGCUAG UCCGUUAUCAACUUGAAAAAGUGGCACCGAGUCGGUGCUUUUUU (SEQ ID NO:110). In some embodiments, the unimolecular, or chimeric, gRNA molecule is a S. pyogenes gRNA molecule.

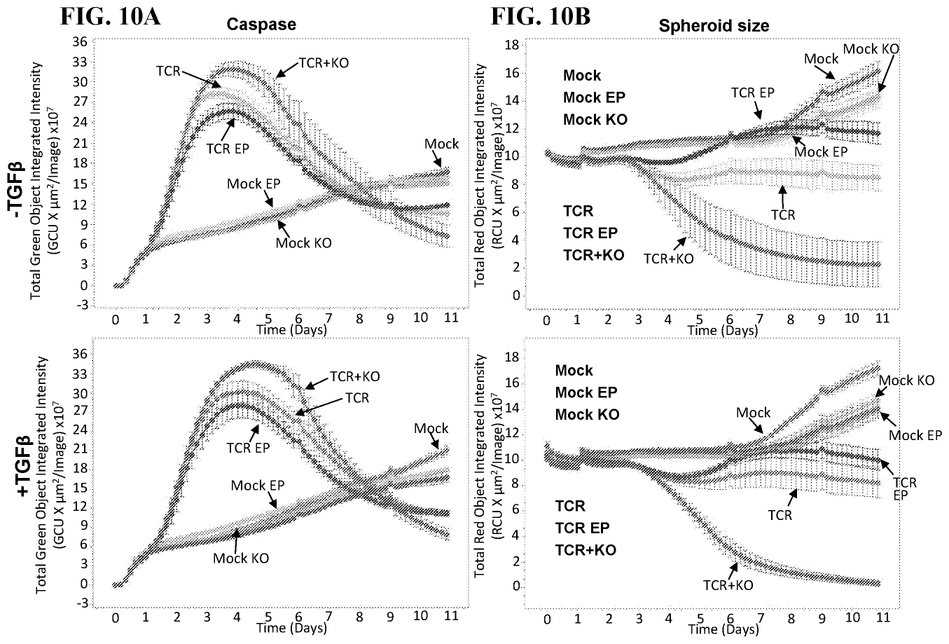

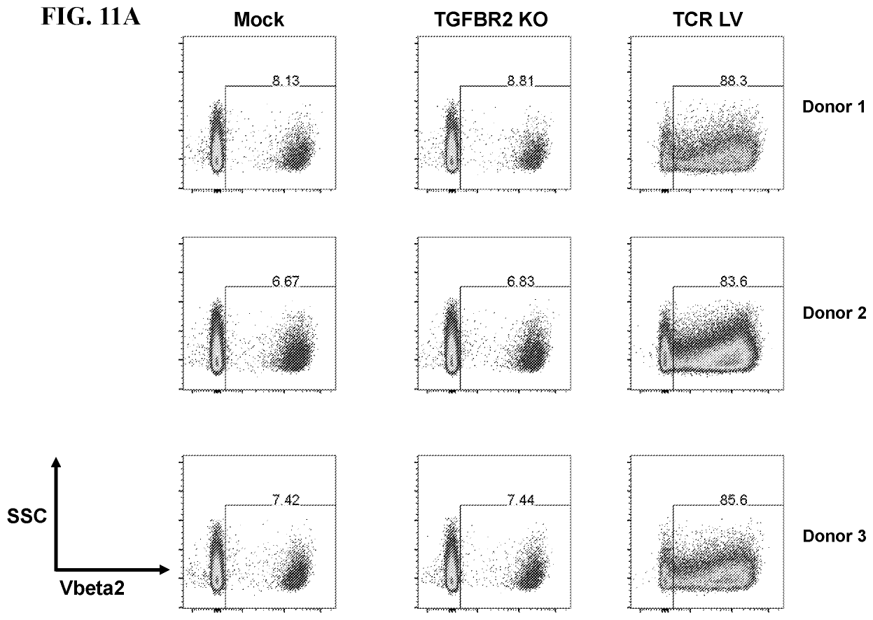

In some embodiments, the unimolecular, or chimeric, gRNA molecule (comprising a targeting domain, a first complementary domain, a linking domain, a second complementary domain, a proximal domain and, optionally, a tail domain) comprises the following sequence in which the targeting domain is depicted as 20 Ns but could be any sequence and range in length from 16 to 26 nucleotides and in which the gRNA sequence is followed by 6 Us, which serve as a termination signal for the U6 promoter, but which could be either absent or fewer in number: NNNNNNNNNNNNNNNNNNNNGUUUUAGUACUCUGGAAACAGAAUCUACUAAAACAAGGC AAAAUGCCGUGUUUAUCUCGUCAACUUGUUGGCGAGAUUUUUU (SEQ ID NO:111). In some embodiments, the unimolecular, or chimeric, gRNA molecule is a S. aureus gRNA molecule. The sequences and structures of exemplary chimeric gRNAs are also shown in WO2015/161276, e.g., in FIGS. 10A-10B therein.

Any of the gRNA molecules as described herein can be used with any Cas9 molecules that generate a double strand break or a single strand break to alter the sequence of a target nucleic acid, e.g., a target position or target genetic signature. In some examples, the target nucleic acid is at or near the TGFBR2 locus, such as any as described. In some embodiments, a ribonucleic acid molecule, such as a gRNA molecule, and a protein, such as a Cas9 protein or variants thereof, are introduced to any of the engineered cells provided herein. gRNA molecules useful in these methods are described below.

In some embodiments, the gRNA, e.g., a chimeric gRNA, is configured such that it comprises one or more of the following properties;

a) it can position, e.g., when targeting a Cas9 molecule that makes double strand breaks, a double strand break (i) within 50, 100, 150, 200, 250, 300, 350, 400, 450, or 500 nucleotides of a target position, or (ii) sufficiently close that the target position is within the region of end resection;

b) it has a targeting domain of at least 16 nucleotides, e.g., a targeting domain of (i) 16, (ii), 17, (iii) 18, (iv) 19, (v) 20, (vi) 21, (vii) 22, (viii) 23, (ix) 24, (x) 25, or (xi) 26 nucleotides; and

c) (i) the proximal and tail domain, when taken together, comprise at least 15, 18, 20, 25, 30, 31, 35, 40, 45, 49, 50, or 53 nucleotides, e.g., at least 15, 18, 20, 25, 30, 31, 35, 40, 45, 49, 50, or 53 nucleotides from a naturally occurring S. pyogenes, S. thermophilus, S. aureus, or N. meningitidis tail and proximal domain, or a sequence that differs by no more than 1, 2, 3, 4, 5, 6, 7, 8, 9 or 10 nucleotides therefrom;

(ii) there are at least 15, 18, 20, 25, 30, 31, 35, 40, 45, 49, 50, or 53 nucleotides 3′ to the last nucleotide of the second complementarity domain, e.g., at least 15, 18, 20, 25, 30, 31, 35, 40, 45, 49, 50, or 53 nucleotides from the corresponding sequence of a naturally occurring S. pyogenes, S. thermophilus, S. aureus, or N. meningitidis gRNA, or a sequence that differs by no more than 1, 2, 3, 4, 5; 6, 7, 8, 9 or 10 nucleotides therefrom;

(iii) there are at least 16, 19, 21, 26, 31, 32, 36, 41, 46, 50, 51, or 54 nucleotides 3′ to the last nucleotide of the second complementarity domain that is complementary to its corresponding nucleotide of the first complementarity domain, e.g., at least 16, 19, 21, 26, 31, 32, 36, 41, 46, 50, 51, or 54 nucleotides from the corresponding sequence of a naturally occurring S. pyogenes, S. thermophilus, S. aureus, or N. meningitidis gRNA, or a sequence that differs by no more than 1, 2, 3, 4, 5; 6, 7, 8, 9 or 10 nucleotides therefrom;

(iv) the tail domain is at least 10, 15, 20, 25, 30, 35 or 40 nucleotides in length, e.g., it comprises at least 10, 15, 20, 25, 30, 35 or 40 nucleotides from a naturally occurring S. pyogenes, S. thermophilus, S. aureus, or N. meningitidis tail domain, or a sequence that differs by no more than 1, 2, 3, 4, 5; 6, 7, 8, 9 or 10 nucleotides therefrom; or

(v) the tail domain comprises 15, 20, 25, 30, 35, 40 nucleotides or all of the corresponding portions of a naturally occurring tail domain, e.g., a naturally occurring S. pyogenes, S. thermophilus, S. aureus, or N. meningitidis tail domain.

In some embodiments, the gRNA is configured such that it comprises properties: a and b(i). In some embodiments, the gRNA is configured such that it comprises properties: a and b(ii). In some embodiments, the gRNA is configured such that it comprises properties: a and b(iii). In some embodiments, the gRNA is configured such that it comprises properties: a and b(iv). In some embodiments, the gRNA is configured such that it comprises properties: a and b(v). In some embodiments, the gRNA is configured such that it comprises properties: a and b(vi). In some embodiments, the gRNA is configured such that it comprises properties: a and b(vii). In some embodiments, the gRNA is configured such that it comprises properties: a and b(viii). In some embodiments, the gRNA is configured such that it comprises properties: a and b(ix). In some embodiments, the gRNA is configured such that it comprises properties: a and b(x). In some embodiments, the gRNA is configured such that it comprises properties: a and b(xi). In some embodiments, the gRNA is configured such that it comprises properties: a and c. In some embodiments, the gRNA is configured such that in comprises properties: a, b, and c. In some embodiments, the gRNA is configured such that in comprises properties: a(i), b(i), and c(i). In some embodiments, the gRNA is configured such that in comprises properties: a(i), b(i), and c(ii). In some embodiments, the gRNA is configured such that in comprises properties: a(i), b(ii), and c(i). In some embodiments, the gRNA is configured such that in comprises properties: a(i), b(ii), and c(ii). In some embodiments, the gRNA is configured such that in comprises properties: a(i), b(iii), and c(i). In some embodiments, the gRNA is configured such that in comprises properties: a(i), b(iii), and c(ii). In some embodiments, the gRNA is configured such that in comprises properties: a(i), b(iv), and c(i). In some embodiments, the gRNA is configured such that in comprises properties: a(i), b(iv), and c(ii). In some embodiments, the gRNA is configured such that in comprises properties: a(i), b(v), and c(i). In some embodiments, the gRNA is configured such that in comprises properties: a(i), b(v), and c(ii). In some embodiments, the gRNA is configured such that in comprises properties: a(i), b(vi), and c(i). In some embodiments, the gRNA is configured such that in comprises properties: a(i), b(vi), and c(ii). In some embodiments, the gRNA is configured such that in comprises properties: a(i), b(vii), and c(i). In some embodiments, the gRNA is configured such that in comprises properties: a(i), b(vii), and c(ii). In some embodiments, the gRNA is configured such that in comprises properties: a(i), b(viii), and c(i). In some embodiments, the gRNA is configured such that in comprises properties: a(i), b(viii), and c(ii). In some embodiments, the gRNA is configured such that in comprises properties: a(i), b(ix), and c(i). In some embodiments, the gRNA is configured such that in comprises properties: a(i), b(ix), and c(ii). In some embodiments, the gRNA is configured such that in comprises properties: a(i), b(x), and c(i). In some embodiments, the gRNA is configured such that in comprises properties: a(i), b(x), and c(ii). In some embodiments, the gRNA is configured such that in comprises properties: a(i), b(xi), and c(i). In some embodiments, the gRNA is configured such that in comprises properties: a(i), b(xi), and c(ii).

In some embodiments, the gRNA, e.g., a chimeric gRNA, is configured such that it comprises one or more of the following properties;

a) one or both of the gRNAs can position, e.g., when targeting a Cas9 molecule that makes single strand breaks, a single strand break within (i) 50, 100, 150, 200, 250, 300, 350, 400, 450, or 500 nucleotides of a target position, or (ii) sufficiently close that the target position is within the region of end resection;

b) one or both have a targeting domain of at least 16 nucleotides, e.g., a targeting domain of (i) 16, (ii), 17, (iii) 18, (iv) 19, (v) 20, (vi) 21, (vii) 22, (viii) 23, (ix) 24, (x) 25, or (xi) 26 nucleotides; and

c) (i) the proximal and tail domain, when taken together, comprise at least 15, 18, 20, 25, 30, 31, 35, 40, 45, 49, 50, or 53 nucleotides, e.g., at least 15, 18, 20, 25, 30, 31, 35, 40, 45, 49, 50, or 53 nucleotides from a naturally occurring S. pyogenes, S. thermophilus, S. aureus, or N. meningitidis tail and proximal domain, or a sequence that differs by no more than 1, 2, 3, 4, 5; 6, 7, 8, 9 or 10 nucleotides therefrom;

(ii) there are at least 15, 18, 20, 25, 30, 31, 35, 40, 45, 49, 50, or 53 nucleotides 3′ to the last nucleotide of the second complementarity domain, e.g., at least 15, 18, 20, 25, 30, 31, 35, 40, 45, 49, 50, or 53 nucleotides from the corresponding sequence of a naturally occurring S. pyogenes, S. thermophilus, S. aureus, or N. meningitidis gRNA, or a sequence that differs by no more than 1, 2, 3, 4, 5; 6, 7, 8, 9 or 10 nucleotides therefrom;

(iii) there are at least 16, 19, 21, 26, 31, 32, 36, 41, 46, 50, 51, or 54 nucleotides 3′ to the last nucleotide of the second complementarity domain that is complementary to its corresponding nucleotide of the first complementarity domain, e.g., at least 16, 19, 21, 26, 31, 32, 36, 41, 46, 50, 51, or 54 nucleotides from the corresponding sequence of a naturally occurring S. pyogenes, S. thermophilus, S. aureus, or N. meningitidis gRNA, or a sequence that differs by no more than 1, 2, 3, 4, 5; 6, 7, 8, 9 or 10 nucleotides therefrom;

(iv) the tail domain is at least 10, 15, 20, 25, 30, 35 or 40 nucleotides in length, e.g., it comprises at least 10, 15, 20, 25, 30, 35 or 40 nucleotides from a naturally occurring S. pyogenes, S. thermophilus, S. aureus, or N. meningitidis tail domain, or a sequence that differs by no more than 1, 2, 3, 4, 5; 6, 7, 8, 9 or 10 nucleotides therefrom; or

(v) the tail domain comprises 15, 20, 25, 30, 35, 40 nucleotides or all of the corresponding portions of a naturally occurring tail domain, e.g., a naturally occurring S. pyogenes, S. thermophilus, S. aureus, or N. meningitidis tail domain.

In some embodiments, the gRNA is configured such that it comprises properties: a and b(i). In some embodiments, the gRNA is configured such that it comprises properties: a and b(ii). In some embodiments, the gRNA is configured such that it comprises properties: a and b(iii). In some embodiments, the gRNA is configured such that it comprises properties: a and b(iv). In some embodiments, the gRNA is configured such that it comprises properties: a and b(v). In some embodiments, the gRNA is configured such that it comprises properties: a and b(vi). In some embodiments, the gRNA is configured such that it comprises properties: a and b(vii). In some embodiments, the gRNA is configured such that it comprises properties: a and b(viii). In some embodiments, the gRNA is configured such that it comprises properties: a and b(ix). In some embodiments, the gRNA is configured such that it comprises properties: a and b(x). In some embodiments, the gRNA is configured such that it comprises properties: a and b(xi). In some embodiments, the gRNA is configured such that it comprises properties: a and c. In some embodiments, the gRNA is configured such that in comprises properties: a, b, and c. In some embodiments, the gRNA is configured such that in comprises properties: a(i), b(i), and c(i). In some embodiments, the gRNA is configured such that in comprises properties: a(i), b(i), and c(ii). In some embodiments, the gRNA is configured such that in comprises properties: a(i), b(ii), and c(i). In some embodiments, the gRNA is configured such that in comprises properties: a(i), b(ii), and c(ii). In some embodiments, the gRNA is configured such that in comprises properties: a(i), b(iii), and c(i). In some embodiments, the gRNA is configured such that in comprises properties: a(i), b(iii), and c(ii). In some embodiments, the gRNA is configured such that in comprises properties: a(i), b(iv), and c(i). In some embodiments, the gRNA is configured such that in comprises properties: a(i), b(iv), and c(ii). In some embodiments, the gRNA is configured such that in comprises properties: a(i), b(v), and c(i). In some embodiments, the gRNA is configured such that in comprises properties: a(i), b(v), and c(ii). In some embodiments, the gRNA is configured such that in comprises properties: a(i), b(vi), and c(i). In some embodiments, the gRNA is configured such that in comprises properties: a(i), b(vi), and c(ii). In some embodiments, the gRNA is configured such that in comprises properties: a(i), b(vii), and c(i). In some embodiments, the gRNA is configured such that in comprises properties: a(i), b(vii), and c(ii). In some embodiments, the gRNA is configured such that in comprises properties: a(i), b(viii), and c(i). In some embodiments, the gRNA is configured such that in comprises properties: a(i), b(viii), and c(ii). In some embodiments, the gRNA is configured such that in comprises properties: a(i), b(ix), and c(i). In some embodiments, the gRNA is configured such that in comprises properties: a(i), b(ix), and c(ii). In some embodiments, the gRNA is configured such that in comprises properties: a(i), b(x), and c(i). In some embodiments, the gRNA is configured such that in comprises properties: a(i), b(x), and c(ii). In some embodiments, the gRNA is configured such that in comprises properties: a(i), b(xi), and c(i). In some embodiments, the gRNA is configured such that in comprises properties: a(i), b(xi), and c(ii).

In some embodiments, the gRNA is used with a Cas9 nickase molecule having HNH activity, e.g., a Cas9 molecule having the RuvC activity inactivated, e.g., a Cas9 molecule having a mutation at D10, e.g., the D10A mutation.

In some embodiments, the gRNA is used with a Cas9 nickase molecule having RuvC activity, e.g., a Cas9 molecule having the HNH activity inactivated, e.g., a Cas9 molecule having a mutation at H840, e.g., a H840A.

In some embodiments, a pair of gRNAs, e.g., a pair of chimeric gRNAs, comprising a first and a second gRNA, is configured such that they comprises one or more of the following properties;

a) one or both of the gRNAs can position, e.g., when targeting a Cas9 molecule that makes single strand breaks, a single strand break within (i) 50, 100, 150, 200, 250, 300, 350, 400, 450, or 500 nucleotides of a target position, or (ii) sufficiently close that the target position is within the region of end resection;

b) one or both have a targeting domain of at least 16 nucleotides, e.g., a targeting domain of (i) 16, (ii), 17, (iii) 18, (iv) 19, (v) 20, (vi) 21, (vii) 22, (viii) 23, (ix) 24, (x) 25, or (xi) 26 nucleotides; c) for one or both:

(i) the proximal and tail domain, when taken together, comprise at least 15, 18, 20, 25, 30, 31, 35, 40, 45, 49, 50, or 53 nucleotides, e.g., at least 15, 18, 20, 25, 30, 31, 35, 40, 45, 49, 50, or 53 nucleotides from a naturally occurring S. pyogenes, S. thermophilus, S. aureus, or N. meningitidis tail and proximal domain, or a sequence that differs by no more than 1, 2, 3, 4, 5; 6, 7, 8, 9 or 10 nucleotides therefrom;

(ii) there are at least 15, 18, 20, 25, 30, 31, 35, 40, 45, 49, 50, or 53 nucleotides 3′ to the last nucleotide of the second complementarity domain, e.g., at least 15, 18, 20, 25, 30, 31, 35, 40, 45, 49, 50, or 53 nucleotides from the corresponding sequence of a naturally occurring S. pyogenes, S. thermophilus, S. aureus, or N. meningitidis gRNA, or a sequence that differs by no more than 1, 2, 3, 4, 5; 6, 7, 8, 9 or 10 nucleotides therefrom;

(iii) there are at least 16, 19, 21, 26, 31, 32, 36, 41, 46, 50, 51, or 54 nucleotides 3′ to the last nucleotide of the second complementarity domain that is complementary to its corresponding nucleotide of the first complementarity domain, e.g., at least 16, 19, 21, 26, 31, 32, 36, 41, 46, 50, 51, or 54 nucleotides from the corresponding sequence of a naturally occurring S. pyogenes, S. thermophilus, S. aureus, or N. meningitidis gRNA, or a sequence that differs by no more than 1, 2, 3, 4, 5; 6, 7, 8, 9 or 10 nucleotides therefrom;

(iv) the tail domain is at least 10, 15, 20, 25, 30, 35 or 40 nucleotides in length, e.g., it comprises at least 10, 15, 20, 25, 30, 35 or 40 nucleotides from a naturally occurring S. pyogenes, S. thermophilus, S. aureus, or N. meningitidis tail domain; or, or a sequence that differs by no more than 1, 2, 3, 4, 5; 6, 7, 8, 9 or 10 nucleotides therefrom; or

(v) the tail domain comprises 15, 20, 25, 30, 35, 40 nucleotides or all of the corresponding portions of a naturally occurring tail domain, e.g., a naturally occurring S. pyogenes, S. thermophilus, S. aureus, or N. meningitidis tail domain;

d) the gRNAs are configured such that, when hybridized to target nucleic acid, they are separated by 0-50, 0-100, 0-200, at least 10, at least 20, at least 30 or at least 50 nucleotides;

e) the breaks made by the first gRNA and second gRNA are on different strands; and

f) the PAMs are facing outwards.

In some embodiments, one or both of the gRNAs is configured such that it comprises properties: a and b(i). In some embodiments, one or both of the gRNAs is configured such that it comprises properties: a and b(ii). In some embodiments, one or both of the gRNAs is configured such that it comprises properties: a and b(iii). In some embodiments, one or both of the gRNAs is configured such that it comprises properties: a and b(iv). In some embodiments, one or both of the gRNAs is configured such that it comprises properties: a and b(v). In some embodiments, one or both of the gRNAs is configured such that it comprises properties: a and b(vi). In some embodiments, one or both of the gRNAs is configured such that it comprises properties: a and b(vii). In some embodiments, one or both of the gRNAs is configured such that it comprises properties: a and b(viii). In some embodiments, one or both of the gRNAs is configured such that it comprises properties: a and b(ix). In some embodiments, one or both of the gRNAs is configured such that it comprises properties: a and b(x). In some embodiments, one or both of the gRNAs is configured such that it comprises properties: a and b(xi). In some embodiments, one or both of the gRNAs configured such that it comprises properties: a and c. In some embodiments, one or both of the gRNAs is configured such that it comprises properties: a, b, and c. In some embodiments, one or both of the gRNAs is configured such that it comprises properties: a(i), b(i), and c(i). In some embodiments, one or both of the gRNAs is configured such that it comprises properties: a(i), b(i), and c(ii). In some embodiments, one or both of the gRNAs is configured such that it comprises properties: a(i), b(i), c, and d. In some embodiments, one or both of the gRNAs is configured such that it comprises properties: a(i), b(i), c, and e. In some embodiments, one or both of the gRNAs is configured such that it comprises properties: a(i), b(i), c, d, and e. In some embodiments, one or both of the gRNAs is configured such that it comprises properties: a(i), b(ii), and c(i). In some embodiments, one or both of the gRNAs is configured such that it comprises properties: a(i), b(ii), and c(ii). In some embodiments, one or both of the gRNAs is configured such that it comprises properties: a(i), b(ii), c, and d. In some embodiments, one or both of the gRNAs is configured such that it comprises properties: a(i), b(ii), c, and e. In some embodiments, one or both of the gRNAs is configured such that it comprises properties: a(i), b(ii), c, d, and e. In some embodiments, one or both of the gRNAs is configured such that it comprises properties: a(i), b(iii), and c(i). In some embodiments, one or both of the gRNAs is configured such that it comprises properties: a(i), b(iii), and c(ii). In some embodiments, one or both of the gRNAs is configured such that it comprises properties: a(i), b(iii), c, and d. In some embodiments, one or both of the gRNAs is configured such that it comprises properties: a(i), b(iii), c, and e. In some embodiments, one or both of the gRNAs is configured such that it comprises properties: a(i), b(iii), c, d, and e. In some embodiments, one or both of the gRNAs is configured such that it comprises properties: a(i), b(iv), and c(i). In some embodiments, one or both of the gRNAs is configured such that it comprises properties: a(i), b(iv), and c(ii). In some embodiments, one or both of the gRNAs is configured such that it comprises properties: a(i), b(iv), c, and d. In some embodiments, one or both of the gRNAs is configured such that it comprises properties: a(i), b(iv), c, and e. In some embodiments, one or both of the gRNAs is configured such that it comprises properties: a(i), b(iv), c, d, and e. In some embodiments, one or both of the gRNAs is configured such that it comprises properties: a(i), b(v), and c(i). In some embodiments, one or both of the gRNAs is configured such that it comprises properties: a(i), b(v), and c(ii). In some embodiments, one or both of the gRNAs is configured such that it comprises properties: a(i), b(v), c, and d. In some embodiments, one or both of the gRNAs is configured such that it comprises properties: a(i), b(v), c, and e. In some embodiments, one or both of the gRNAs is configured such that it comprises properties: a(i), b(v), c, d, and e. In some embodiments, one or both of the gRNAs is configured such that it comprises properties: a(i), b(vi), and c(i). In some embodiments, one or both of the gRNAs is configured such that it comprises properties: a(i), b(vi), and c(ii). In some embodiments, one or both of the gRNAs is configured such that it comprises properties: a(i), b(vi), c, and d. In some embodiments, one or both of the gRNAs is configured such that it comprises properties: a(i), b(vi), c, and e. In some embodiments, one or both of the gRNAs is configured such that it comprises properties: a(i), b(vi), c, d, and e. In some embodiments, one or both of the gRNAs is configured such that it comprises properties: a(i), b(vii), and c(i). In some embodiments, one or both of the gRNAs is configured such that it comprises properties: a(i), b(vii), and c(ii). In some embodiments, one or both of the gRNAs is configured such that it comprises properties: a(i), b(vii), c, and d. In some embodiments, one or both of the gRNAs is configured such that it comprises properties: a(i), b(vii), c, and e. In some embodiments, one or both of the gRNAs is configured such that it comprises properties: a(i), b(vii), c, d, and e. In some embodiments, one or both of the gRNAs is configured such that it comprises properties: a(i), b(viii), and c(i). In some embodiments, one or both of the gRNAs is configured such that it comprises properties: a(i), b(viii), and c(ii). In some embodiments, one or both of the gRNAs is configured such that it comprises properties: a(i), b(viii), c, and d. In some embodiments, one or both of the gRNAs is configured such that it comprises properties: a(i), b(viii), c, and e. In some embodiments, one or both of the gRNAs is configured such that it comprises properties: a(i), b(viii), c, d, and e. In some embodiments, one or both of the gRNAs is configured such that it comprises properties: a(i), b(ix), and c(i). In some embodiments, one or both of the gRNAs is configured such that it comprises properties: a(i), b(ix), and c(ii). In some embodiments, one or both of the gRNAs is configured such that it comprises properties: a(i), b(ix), c, and d. In some embodiments, one or both of the gRNAs is configured such that it comprises properties: a(i), b(ix), c, and e. In some embodiments, one or both of the gRNAs is configured such that it comprises properties: a(i), b(ix), c, d, and e. In some embodiments, one or both of the gRNAs is configured such that it comprises properties: a(i), b(x), and c(i). In some embodiments, one or both of the gRNAs is configured such that it comprises properties: a(i), b(x), and c(ii). In some embodiments, one or both of the gRNAs is configured such that it comprises properties: a(i), b(x), c, and d. In some embodiments, one or both of the gRNAs is configured such that it comprises properties: a(i), b(x), c, and e. In some embodiments, one or both of the gRNAs is configured such that it comprises properties: a(i), b(x), c, d, and e. In some embodiments, one or both of the gRNAs is configured such that it comprises properties: a(i), b(xi), and c(i). In some embodiments, one or both of the gRNAs is configured such that it comprises properties: a(i), b(xi), and c(ii). In some embodiments, one or both of the gRNAs is configured such that it comprises properties: a(i), b(xi), c, and d. In some embodiments, one or both of the gRNAs is configured such that it comprises properties: a(i), b(xi), c, and e. In some embodiments, one or both of the gRNAs is configured such that it comprises properties: a(i), b(xi), c, d, and e.

In some embodiments, the gRNAs are used with a Cas9 nickase molecule having HNH activity, e.g., a Cas9 molecule having the RuvC activity inactivated, e.g., a Cas9 molecule having a mutation at D10, e.g., the D10A mutation.

In some embodiments, the gRNAs are used with a Cas9 nickase molecule having RuvC activity, e.g., a Cas9 molecule having the HNH activity inactivated, e.g., a Cas9 molecule having a mutation at H840, e.g., a H840A. In some embodiments, the gRNAs are used with a Cas9 nickase molecule having RuvC activity, e.g., a Cas9 molecule having the HNH activity inactivated, e.g., a Cas9 molecule having a mutation at N863, e.g., N863A.

(j) Exemplary Modular gRNAs

In some embodiments, a modular gRNA comprises first and second strands. The first strand comprises, preferably from 5′ to 3′; a targeting domain, e.g., comprising 15, 16, 17, 18, 19, 20, 21, 22, 23, 24, 25, or 26 nucleotides; a first complementarity domain. The second strand comprises, preferably from 5′ to 3′: optionally a 5′ extension domain; a second complementarity domain; a proximal domain; and a tail domain, wherein: (a) the proximal and tail domain, when taken together, comprise at least 15, 18, 20, 25, 30, 31, 35, 40, 45, 49, 50, or 53 nucleotides; (b) there are at least 15, 18, 20, 25, 30, 31, 35, 40, 45, 49, 50, or 53 nucleotides 3′ to the last nucleotide of the second complementarity domain; or (c) there are at least 16, 19, 21, 26, 31, 32, 36, 41, 46, 50, 51, or 54 nucleotides 3′ to the last nucleotide of the second complementarity domain that is complementary to its corresponding nucleotide of the first complementarity domain.

In some embodiments, the sequence from (a), (b), or (c), has at least 60, 75, 80, 85, 90, 95, or 99% homology with the corresponding sequence of a naturally occurring gRNA, or with a gRNA described herein. In some embodiments, the proximal and tail domain, when taken together, comprise at least 15, 18, 20, 25, 30, 31, 35, 40, 45, 49, 50, or 53 nucleotides. In some embodiments there are at least 15, 18, 20, 25, 30, 31, 35, 40, 45, 49, 50, or 53 nucleotides 3′ to the last nucleotide of the second complementarity domain.

In some embodiments, there are at least 16, 19, 21, 26, 31, 32, 36, 41, 46, 50, 51, or 54 nucleotides 3′ to the last nucleotide of the second complementarity domain that is complementary to its corresponding nucleotide of the first complementarity domain.

In some embodiments, the targeting domain has, or consists of, 16, 17, 18, 19, 20, 21, 22, 23, 24, 25 or 26 nucleotides (e.g., 16, 17, 18, 19, 20, 21, 22, 23, 24, 25 or 26 consecutive nucleotides) having complementarity with the target domain, e.g., the targeting domain is 16, 17, 18, 19, 20, 21, 22, 23, 24, 25 or 26 nucleotides in length.

(k) Methods for Designing gRNAs

Methods for designing gRNAs are described herein, including methods for selecting, designing and validating targeting domains. Exemplary targeting domains are also provided herein.

Targeting domains discussed herein can be incorporated into the gRNAs described herein.

Methods for selection and validation of target sequences as well as off-target analyses are described, e.g., in Mali et al., 2013 Science 339(6121): 823-826; Hsu et al. Nat Biotechnol, 31(9): 827-32; Fu et al., 2014 Nat Biotechnol, doi: 10.1038/nbt.2808. PubMed PMID: 24463574; Heigwer et al., 2014 Nat Methods 11(2):122-3. doi: 10.1038/nmeth.2812. PubMed PMID: 24481216; Bae et al., 2014 Bioinformatics PubMed PMID: 24463181; Xiao A et al., 2014 Bioinformatics PubMed PMID: 24389662.

In some embodiments, a software tool can be used to optimize the choice of gRNA within a user's target sequence, e.g., to minimize total off-target activity across the genome. Off target activity may be other than cleavage. For example, for each possible gRNA choice using S. pyogenes Cas9, software tools can identify all potential off-target sequences (preceding either NAG or NGG PAMs) across the genome that contain up to a certain number (e.g., 1, 2, 3, 4, 5, 6, 7, 8, 9, or 10) of mismatched base-pairs. The cleavage efficiency at each off-target sequence can be predicted, e.g., using an experimentally-derived weighting scheme. Each possible gRNA can then be ranked according to its total predicted off-target cleavage; the top-ranked gRNAs represent those that are likely to have the greatest on-target and the least off-target cleavage. Other functions, e.g., automated reagent design for gRNA vector construction, primer design for the on-target Surveyor assay, and primer design for high-throughput detection and quantification of off-target cleavage via next-generation sequencing, can also be included in the tool. Candidate gRNA molecules can be evaluated by art-known methods or as described herein.

In some embodiments, gRNAs for use with S. pyogenes, S. aureus, and N. meningitidis Cas9s are identified using a DNA sequence searching algorithm, e.g., using a custom gRNA design software based on the public tool cas-offinder (Bae et al. Bioinformatics. 2014; 30(10): 1473-1475). The custom gRNA design software scores guides after calculating their genome-wide off-target propensity. Typically matches ranging from perfect matches to 7 mismatches are considered for guides ranging in length from 17 to 24. In some aspects, once the off-target sites are computationally determined, an aggregate score is calculated for each guide and summarized in a tabular output using a web-interface. In addition to identifying potential gRNA sites adjacent to PAM sequences, the software also can identify all PAM adjacent sequences that differ by 1, 2, 3 or more nucleotides from the selected gRNA sites. In some embodiments, genomic DNA sequences for each gene are obtained from the UCSC Genome browser and sequences can be screened for repeat elements using the publicly available RepeatMasker program. RepeatMasker searches input DNA sequences for repeated elements and regions of low complexity. The output is a detailed annotation of the repeats present in a given query sequence.

Following identification, gRNAs can be ranked into tiers based on one or more of their distance to the target site, their orthogonality and presence of a 5′ G (based on identification of close matches in the human genome containing a relevant PAM, e.g., in the case of S. pyogenes, a NGG PAM, in the case of S. aureus, NNGRR (e.g., a NNGRRT or NNGRRV) PAM, and in the case of N. meningtidis, a NNNNGATT or NNNNGCTT PAM). Orthogonality refers to the number of sequences in the human genome that contain a minimum number of mismatches to the target sequence. A “high level of orthogonality” or “good orthogonality” may, for example, refer to 20-mer targeting domains that have no identical sequences in the human genome besides the intended target, nor any sequences that contain one or two mismatches in the target sequence. Targeting domains with good orthogonality are selected to minimize off-target DNA cleavage. It is to be understood that this is a non-limiting example and that a variety of strategies could be utilized to identify gRNAs for use with S. pyogenes, S. aureus and N. meningitidis or other Cas9 enzymes.

In some embodiments, gRNAs for use with the S. pyogenes Cas9 can be identified using the publicly available web-based ZiFiT server (Fu et al., Improving CRISPR-Cas nuclease specificity using truncated guide RNAs. Nat Biotechnol. 2014 Jan. 26. doi: 10.1038/nbt.2808. PubMed PMID: 24463574, for the original references see Sander et al., 2007, NAR 35:W599-605; Sander et al., 2010, NAR 38: W462-8). In addition to identifying potential gRNA sites adjacent to PAM sequences, the software also identifies all PAM adjacent sequences that differ by 1, 2, 3 or more nucleotides from the selected gRNA sites. In some aspects, genomic DNA sequences for each gene can be obtained from the UCSC Genome browser and sequences can be screened for repeat elements using the publicly available Repeat-Masker program. RepeatMasker searches input DNA sequences for repeated elements and regions of low complexity. The output is a detailed annotation of the repeats present in a given query sequence.

Following identification, gRNAs for use with a S. pyogenes Cas9 can be ranked into tiers, e.g. into 5 tiers. In some embodiments, the targeting domains for first tier gRNA molecules are selected based on their distance to the target site, their orthogonality and presence of a 5′ G (based on the ZiFiT identification of close matches in the human genome containing an NGG PAM). In some embodiments, both 17-mer and 20-mer gRNAs are designed for targets. In some aspects, gRNAs are also selected both for single-gRNA nuclease cutting and for the dual gRNA nickase strategy. Criteria for selecting gRNAs and the determination for which gRNAs can be used for which strategy can be based on several considerations. In some embodiments, gRNAs for both single-gRNA nuclease cleavage and for a dual-gRNA paired “nickase” strategy are identified. In some embodiments for selecting gRNAs, including the determination for which gRNAs can be used for the dual-gRNA paired “nickase” strategy, gRNA pairs should be oriented on the DNA such that PAMs are facing out and cutting with the D10A Cas9 nickase will result in 5′ overhangs. In some aspects, it can be assumed that cleaving with dual nickase pairs will result in deletion of the entire intervening sequence at a reasonable frequency. However, cleaving with dual nickase pairs can also often result in indel mutations at the site of only one of the gRNAs. Candidate pair members can be tested for how efficiently they remove the entire sequence versus just causing indel mutations at the site of one gRNA.

In some embodiments, the targeting domains for first tier gRNA molecules can be selected based on (1) a reasonable distance to the target position, e.g., within the first 500 bp of coding sequence downstream of start codon, (2) a high level of orthogonality, and (3) the presence of a 5′ G. In some embodiments, for selection of second tier gRNAs, the requirement for a 5′G can be removed, but the distance restriction is required and a high level of orthogonality was required. In some embodiments, third tier selection uses the same distance restriction and the requirement for a 5′G, but removes the requirement of good orthogonality. In some embodiments, fourth tier selection uses the same distance restriction but removes the requirement of good orthogonality and start with a 5′G. In some embodiments, fifth tier selection removes the requirement of good orthogonality and a 5′G, and a longer sequence (e.g., the rest of the coding sequence, e.g., additional 500 bp upstream or downstream to the transcription target site) is scanned. In certain instances, no gRNA is identified based on the criteria of the particular tier.

In some embodiments, gRNAs are identified for single-gRNA nuclease cleavage as well as for a dual-gRNA paired “nickase” strategy.

In some aspects, gRNAs for use with the N. meningitidis and S. aureus Cas9s can be identified manually by scanning genomic DNA sequence for the presence of PAM sequences. These gRNAs can be separated into two tiers. In some embodiments, for first tier gRNAs, targeting domains are selected within the first 500 bp of coding sequence downstream of start codon. In some embodiments, for second tier gRNAs, targeting domains are selected within the remaining coding sequence (downstream of the first 500 bp). In certain instances, no gRNA is identified based on the criteria of the particular tier.

In some embodiments, another strategy for identifying guide RNAs (gRNAs) for use with S. pyogenes, S. aureus and N. meningtidis Cas9s can use a DNA sequence searching algorithm. In some aspects, guide RNA design is carried out using a custom guide RNA design software based on the public tool cas-offinder (Bae et al. Bioinformatics. 2014; 30(10): 1473-1475). Said custom guide RNA design software scores guides after calculating their genome wide off-target propensity. Typically matches ranging from perfect matches to 7 mismatches are considered for guides ranging in length from 17 to 24. Once the off-target sites are computationally determined, an aggregate score is calculated for each guide and summarized in a tabular output using a web-interface. In addition to identifying potential gRNA sites adjacent to PAM sequences, the software also identifies all PAM adjacent sequences that differ by 1, 2, 3 or more nucleotides from the selected gRNA sites. In some embodiments, genomic DNA sequence for each gene is obtained from the UCSC Genome browser and sequences are screened for repeat elements using the publically available RepeatMasker program. RepeatMasker searches input DNA sequences for repeated elements and regions of low complexity. The output is a detailed annotation of the repeats present in a given query sequence.

In some embodiments, following identification, gRNAs are ranked into tiers based on their distance to the target site or their orthogonality (based on identification of close matches in the human genome containing a relevant PAM, e.g., in the case of S. pyogenes, a NGG PAM, in the case of S. aureus, NNGRR (e.g., a NNGRRT or NNGRRV) PAM, and in the case of N. meningtidis, a NNNNGATT or NNNNGCTT PAM. In some aspects, targeting domains with good orthogonality are selected to minimize off-target DNA cleavage.

As an example, for S. pyogenes and N. meningtidis targets, 17-mer, or 20-mer gRNAs can be designed. As another example, for S. aureus targets, 18-mer, 19-mer, 20-mer, 21-mer, 22-mer, 23-mer and 24-mer gRNAs can be designed.

In some embodiments, gRNAs for both single-gRNA nuclease cleavage and for a dual-gRNA paired “nickase” strategy are identified. In some embodiments for selecting gRNAs, including the determination for which gRNAs can be used for the dual-gRNA paired “nickase” strategy, gRNA pairs should be oriented on the DNA such that PAMs are facing out and cutting with the D10A Cas9 nickase will result in 5′ overhangs. In some aspects, it can be assumed that cleaving with dual nickase pairs will result in deletion of the entire intervening sequence at a reasonable frequency. However, cleaving with dual nickase pairs can also often result in indel mutations at the site of only one of the gRNAs. Candidate pair members can be tested for how efficiently they remove the entire sequence versus just causing indel mutations at the site of one gRNA.

For designing strategies for genetic disruption, in some embodiments, the targeting domains for tier 1 gRNA molecules for S. pyogenes are selected based on their distance to the target site and their orthogonality (PAM is NGG). In some cases, the targeting domains for tier 1 gRNA molecules are selected based on (1) a reasonable distance to the target position, e.g., within the first 500 bp of coding sequence downstream of start codon and (2) a high level of orthogonality. In some aspects, for selection of tier 2 gRNAs, a high level of orthogonality is not required. In some cases, tier 3 gRNAs remove the requirement of good orthogonality and a longer sequence (e.g., the rest of the coding sequence) can be scanned. In certain instances, no gRNA is identified based on the criteria of the particular tier.

For designing strategies for genetic disruption, in some embodiments, the targeting domain for tier 1 gRNA molecules for N. meningtidis were selected within the first 500 bp of the coding sequence and had a high level of orthogonality. The targeting domain for tier 2 gRNA molecules for N. meningtidis were selected within the first 500 bp of the coding sequence and did not require high orthogonality. The targeting domain for tier 3 gRNA molecules for N. meningtidis were selected within a remainder of coding sequence downstream of the 500 bp. Note that tiers are non-inclusive (each gRNA is listed only once). In certain instances, no gRNA was identified based on the criteria of the particular tier.

For designing strategies for genetic disruption, in some embodiments, the targeting domain for tier 1 gRNA molecules for S. aureus is selected within the first 500 bp of the coding sequence, has a high level of orthogonality, and contains a NNGRRT PAM. In some embodiments, the targeting domain for tier 2 gRNA molecules for S. aureus is selected within the first 500 bp of the coding sequence, no level of orthogonality is required, and contains a NNGRRT PAM. In some embodiments, the targeting domain for tier 3 gRNA molecules for S. aureus are selected within the remainder of the coding sequence downstream and contain a NNGRRT PAM. In some embodiments, the targeting domain for tier 4 gRNA molecules for S. aureus are selected within the first 500 bp of the coding sequence and contain a NNGRRV PAM. In some embodiments, the targeting domain for tier 5 gRNA molecules for S. aureus are selected within the remainder of the coding sequence downstream and contain a NNGRRV PAM. In certain instances, no gRNA is identified based on the criteria of the particular tier.

(ii) Cas9

Cas9 molecules of a variety of species can be used in the methods and compositions described herein. While the S. pyogenes, S. aureus, N. meningitidis, and S. thermophilus Cas9 molecules are the subject of much of the disclosure herein, Cas9 molecules of, derived from, or based on the Cas9 proteins of other species listed herein can be used as well. In other words, while the much of the description herein uses S. pyogenes, S. aureus, N. meningitidis, and S. thermophilus Cas9 molecules, Cas9 molecules from the other species can replace them. Such species include: Acidovorax avenae, Actinobacillus pleuropneumoniae, Actinobacillus succinogenes, Actinobacillus suis, Actinomyces sp., Cycliphilus denitrificans, Aminomonas paucivorans, Bacillus cereus, Bacillus smithii, Bacillus thuringiensis, Bacteroides sp., Blastopirellula marina, Bradyrhizobium sp., Brevibacillus laterosporus, Campylobacter coli, Campylobacter jejuni, Campylobacter lari, Candidatus puniceispirillum, Clostridium cellulolyticum, Clostridium perfringens, Corynebacterium accolens, Corynebacterium diphtheria, Corynebacterium matruchotii, Dinoroseobacter shibae, Eubacterium dolichum, Gammaproteobacterium, Gluconacetobacter diazotrophicus, Haemophilus parainfluenzae, Haemophilus sputorum, Helicobacter canadensis, Helicobacter cinaedi, Helicobacter mustelae, Ilyobacter polytropus, Kingella kingae, Lactobacillus crispatus, Listeria ivanovii, Listeria monocytogenes, Listeriaceae bacterium, Methylocystis sp., Methylosinus trichosporium, Mobiluncus mulieris, Neisseria bacilliformis, Neisseria cinerea, Neisseria flavescens, Neisseria lactamica, Neisseria meningitidis, Neisseria sp., Neisseria wadsworthii, Nitrosomonas sp., Parvibaculum lavamentivorans, Pasteurella multocida, Phascolarctobacterium succinatutens, Ralstonia syzygii, Rhodopseudomonas palustris, Rhodovulum sp., Simonsiella muelleri, Sphingomonas sp., Sporolactobacillus vineae, Staphylococcus aureus, Staphylococcus lugdunensis, Streptococcus sp., Subdoligranulum sp., Tistrella mobilis, Treponema sp., or Verminephrobacter eiseniae. Examples of Cas9 molecules can include those described in, e.g., WO2015/161276, WO2017/193107, WO2017/093969, US2016/272999 and US2015/056705.

A Cas9 molecule, or Cas9 polypeptide, as that term is used herein, refers to a molecule or polypeptide that can interact with a gRNA molecule and, in concert with the gRNA molecule, homes or localizes to a site which comprises a target domain and PAM sequence. Cas9 molecule and Cas9 polypeptide, as those terms are used herein, refer to naturally occurring Cas9 molecules and to engineered, altered, or modified Cas9 molecules or Cas9 polypeptides that differ, e.g., by at least one amino acid residue, from a reference sequence, e.g., the most similar naturally occurring Cas9 molecule.

Crystal structures have been determined for two different naturally occurring bacterial Cas9 molecules (Jinek et al., Science, 343(6176):1247997, 2014) and for S. pyogenes Cas9 with a guide RNA (e.g., a synthetic fusion of crRNA and tracrRNA) (Nishimasu et al., Cell, 156:935-949, 2014; and Anders et al., Nature, 2014, doi: 10.1038/nature13579).

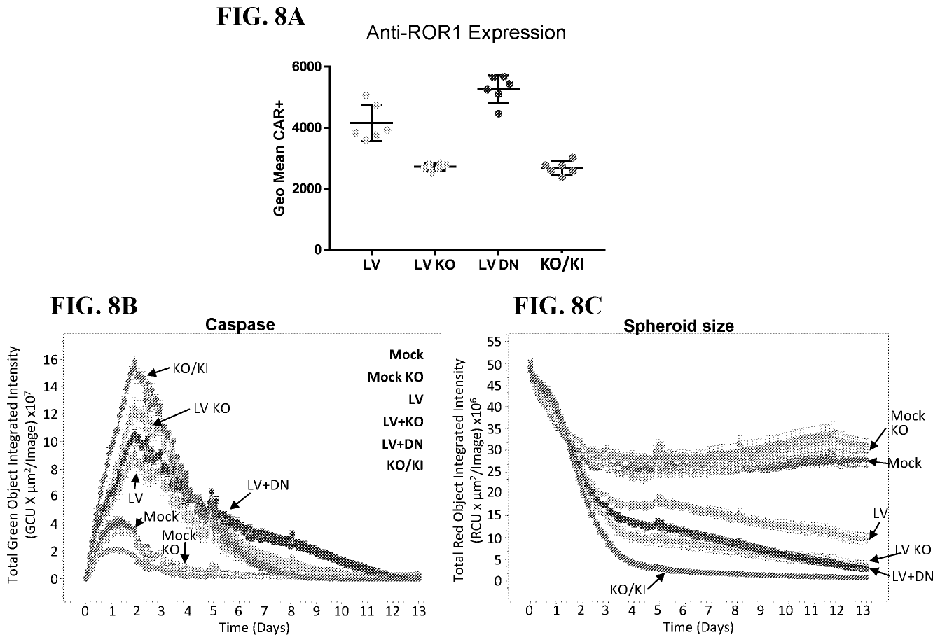

A naturally occurring Cas9 molecule comprises two lobes: a recognition (REC) lobe and a nuclease (NUC) lobe; each of which further comprises domains described herein. An exemplary schematic of the organization of important Cas9 domains in the primary structure is described in WO2015/161276, e.g., in FIGS. 8A-8B therein. The domain nomenclature and the numbering of the amino acid residues encompassed by each domain used throughout this disclosure is as described in Nishimasu et al. The numbering of the amino acid residues is with reference to Cas9 from S. pyogenes.

The REC lobe comprises the arginine-rich bridge helix (BH), the REC1 domain, and the REC2 domain. The REC lobe does not share structural similarity with other known proteins, indicating that it is a Cas9-specific functional domain. The BH domain is a long α-helix and arginine rich region and comprises amino acids 60-93 of the sequence of S. pyogenes Cas9. The REC1 domain is important for recognition of the repeat:anti-repeat duplex, e.g., of a gRNA or a tracrRNA, and is therefore critical for Cas9 activity by recognizing the target sequence. The REC1 domain comprises two REC1 motifs at amino acids 94 to 179 and 308 to 717 of the sequence of S. pyogenes Cas9. These two REC1 domains, though separated by the REC2 domain in the linear primary structure, assemble in the tertiary structure to form the REC1 domain. The REC2 domain, or parts thereof, may also play a role in the recognition of the repeat:anti-repeat duplex. The REC2 domain comprises amino acids 180-307 of the sequence of S. pyogenes Cas9.

The NUC lobe comprises the RuvC domain (also referred to herein as RuvC-like domain), the HNH domain (also referred to herein as HNH-like domain), and the PAM-interacting (PI) domain. The RuvC domain shares structural similarity to retroviral integrase superfamily members and cleaves a single strand, e.g., the non-complementary strand of the target nucleic acid molecule. The RuvC domain is assembled from the three split RuvC motifs (RuvC I, RuvCII, and RuvCIII, which are often commonly referred to as RuvCI domain, or N-terminal RuvC domain, RuvCII domain, and RuvCIII domain) at amino acids 1-59, 718-769, and 909-1098, respectively, of the sequence of S. pyogenes Cas9. Similar to the REC1 domain, the three RuvC motifs are linearly separated by other domains in the primary structure, however in the tertiary structure, the three RuvC motifs assemble and form the RuvC domain. The HNH domain shares structural similarity with HNH endonucleases, and cleaves a single strand, e.g., the complementary strand of the target nucleic acid molecule. The HNH domain lies between the RuvC II-III motifs and comprises amino acids 775-908 of the sequence of S. pyogenes Cas9. The PI domain interacts with the PAM of the target nucleic acid molecule, and comprises amino acids 1099-1368 of the sequence of S. pyogenes Cas9.

(a) A RuvC-Like Domain and an HNH-Like Domain

In some embodiments, a Cas9 molecule or Cas9 polypeptide comprises an HNH-like domain and a RuvC-like domain. In some embodiments, cleavage activity is dependent on a RuvC-like domain and an HNH-like domain. A Cas9 molecule or Cas9 polypeptide, e.g., an eaCas9 molecule or eaCas9 polypeptide, can comprise one or more of the following domains: a RuvC-like domain and an HNH-like domain. In some embodiments, a Cas9 molecule or Cas9 polypeptide is an eaCas9 molecule or eaCas9 polypeptide and the eaCas9 molecule or eaCas9 polypeptide comprises a RuvC-like domain, e.g., a RuvC-like domain described herein, and/or an HNH-like domain, e.g., an HNH-like domain described herein.

(b) RuvC-Like Domains

In some embodiments, a RuvC-like domain cleaves, a single strand, e.g., the non-complementary strand of the target nucleic acid molecule. The Cas9 molecule or Cas9 polypeptide can include more than one RuvC-like domain (e.g., one, two, three or more RuvC-like domains). In some embodiments, a RuvC-like domain is at least 5, 6, 7, 8 amino acids in length but not more than 20, 19, 18, 17, 16 or 15 amino acids in length. In some embodiments, the Cas9 molecule or Cas9 polypeptide comprises an N-terminal RuvC-like domain of about 10 to 20 amino acids, e.g., about 15 amino acids in length.

(c) N-Terminal RuvC-Like Domains

Some naturally occurring Cas9 molecules comprise more than one RuvC-like domain with cleavage being dependent on the N-terminal RuvC-like domain. Accordingly, Cas9 molecules or Cas9 polypeptide can comprise an N-terminal RuvC-like domain.

In embodiment, the N-terminal RuvC-like domain is cleavage competent.

In embodiment, the N-terminal RuvC-like domain is cleavage incompetent.

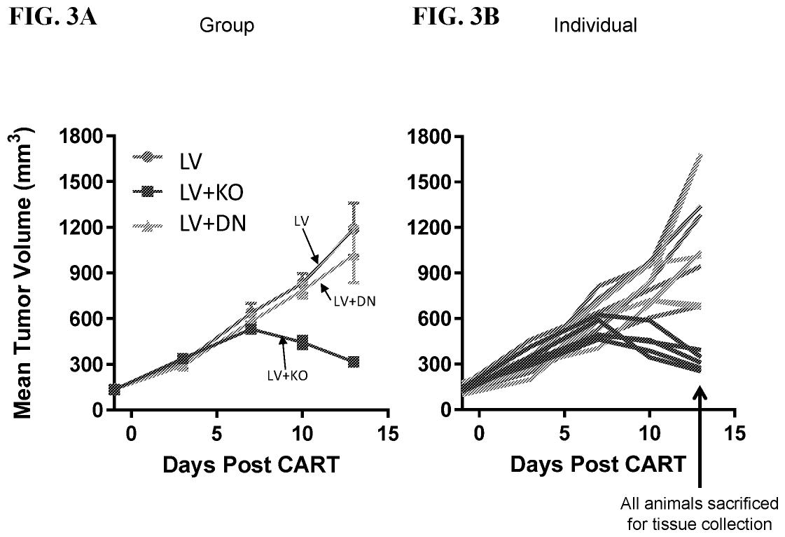

In some embodiments, the N-terminal RuvC-like domain differs from a sequence of an N-terminal RuvC like domain disclosed herein, e.g., in WO2015/161276, e.g., in FIGS. 3A-3B or FIGS. 7A-7B therein, as many as 1 but no more than 2, 3, 4, or 5 residues. In some embodiments, 1, 2, or all 3 of the highly conserved residues identified WO2015/161276, e.g., in FIGS. 3A-3B or FIGS. 7A-7B therein are present.

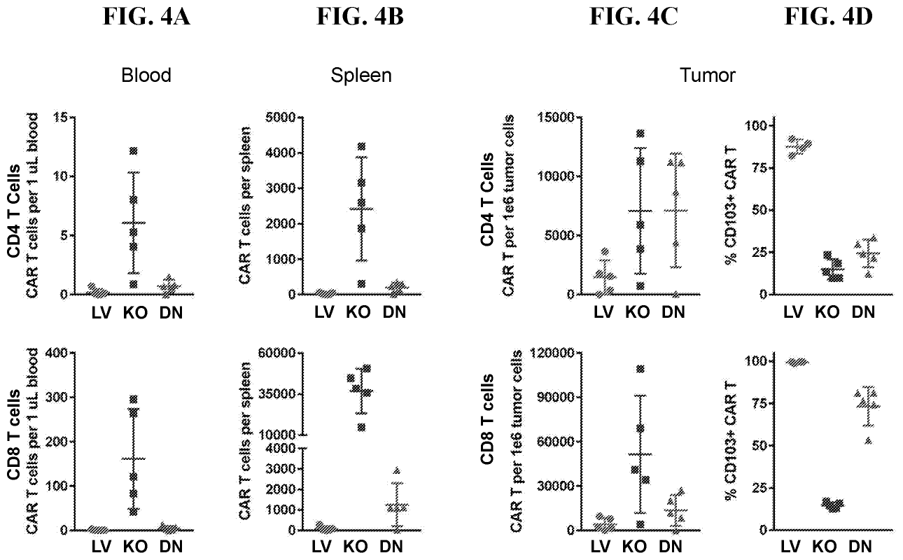

In some embodiments, the N-terminal RuvC-like domain differs from a sequence of an N-terminal RuvC-like domain disclosed herein, e.g., in WO2015/161276, e.g., in FIGS. 4A-4B or FIGS. 7A-7B therein, as many as 1 but no more than 2, 3, 4, or 5 residues. In some embodiments, 1, 2, 3 or all 4 of the highly conserved residues identified in WO2015/161276, e.g., in FIGS. 4A-4B or FIGS. 7A-7B therein are present.

(d) Additional RuvC-Like Domains

In addition to the N-terminal RuvC-like domain, the Cas9 molecule or Cas9 polypeptide, e.g., an eaCas9 molecule or eaCas9 polypeptide, can comprise one or more additional RuvC-like domains. In some embodiments, the Cas9 molecule or Cas9 polypeptide can comprise two additional RuvC-like domains. Preferably, the additional RuvC-like domain is at least 5 amino acids in length and, e.g., less than 15 amino acids in length, e.g., 5 to 10 amino acids in length, e.g., 8 amino acids in length.

(e) HNH-Like Domains

In some embodiments, an HNH-like domain cleaves a single stranded complementary domain, e.g., a complementary strand of a double stranded nucleic acid molecule. In some embodiments, an HNH-like domain is at least 15, 20, 25 amino acids in length but not more than 40, 35 or 30 amino acids in length, e.g., 20 to 35 amino acids in length, e.g., 25 to 30 amino acids in length. Exemplary HNH-like domains are described herein.

In some embodiments, the HNH-like domain is cleavage competent.

In some embodiments, the HNH-like domain is cleavage incompetent.

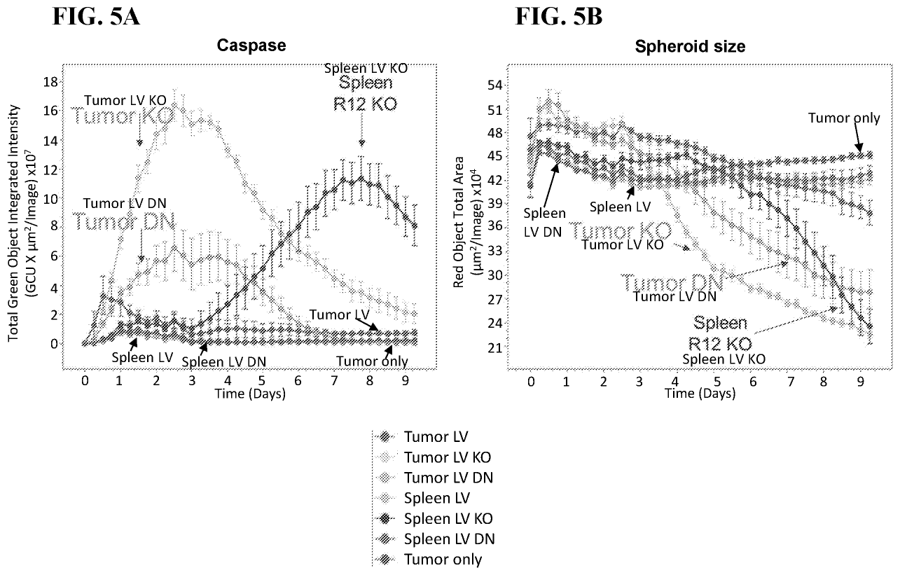

In some embodiments, the HNH-like domain differs from a sequence of an HNH-like domain disclosed herein, e.g., in WO2015/161276, e.g., in FIGS. 5A-5C or FIGS. 7A-7B therein, as many as 1 but no more than 2, 3, 4, or 5 residues. In some embodiments, 1 or both of the highly conserved residues identified in WO2015/161276, e.g., in FIGS. 5A-5C or FIGS. 7A-7B therein are present.

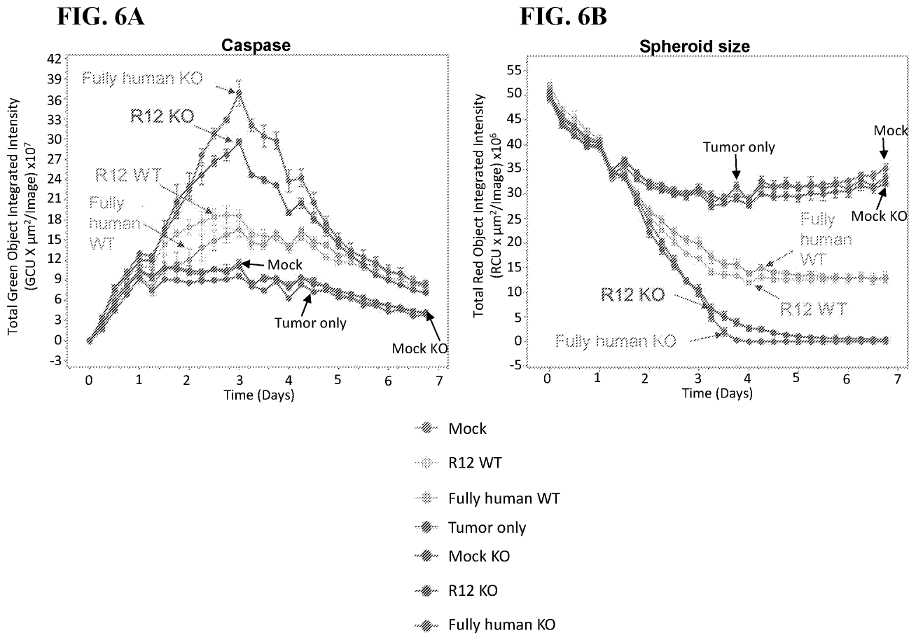

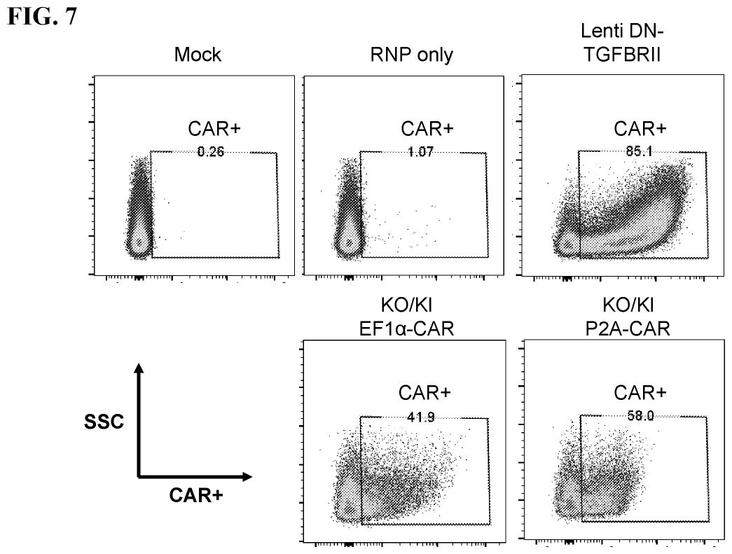

In some embodiments, the HNH-like domain differs from a sequence of an HNH-like domain disclosed herein, e.g., in WO2015/161276, e.g., in FIGS. 6A-6B or FIGS. 7A-7B therein, as many as 1 but no more than 2, 3, 4, or 5 residues. In some embodiments, 1, 2, all 3 of the highly conserved residues identified in WO2015/161276, e.g., in FIGS. 6A-6B or FIGS. 7A-7B therein are present.

(f) Nuclease and Helicase Activities

In some embodiments, the Cas9 molecule or Cas9 polypeptide is capable of cleaving a target nucleic acid molecule. Typically wild type Cas9 molecules cleave both strands of a target nucleic acid molecule. Cas9 molecules and Cas9 polypeptides can be engineered to alter nuclease cleavage (or other properties), e.g., to provide a Cas9 molecule or Cas9 polypeptide which is a nickase, or which lacks the ability to cleave target nucleic acid. A Cas9 molecule or Cas9 polypeptide that is capable of cleaving a target nucleic acid molecule is referred to herein as an eaCas9 molecule or eaCas9 polypeptide.

In some embodiments, an eaCas9 molecule or eaCas9 polypeptide comprises one or more of the following activities: a nickase activity, i.e., the ability to cleave a single strand, e.g., the non-complementary strand or the complementary strand, of a nucleic acid molecule; a double stranded nuclease activity, i.e., the ability to cleave both strands of a double stranded nucleic acid and create a double stranded break, which In some embodiments is the presence of two nickase activities; an endonuclease activity; an exonuclease activity; and a helicase activity, i.e., the ability to unwind the helical structure of a double stranded nucleic acid.

In some embodiments, an enzymatically active or eaCas9 molecule or eaCas9 polypeptide cleaves both strands and results in a double stranded break. In some embodiments, an eaCas9 molecule cleaves only one strand, e.g., the strand to which the gRNA hybridizes to, or the strand complementary to the strand the gRNA hybridizes with. In some embodiments, an eaCas9 molecule or eaCas9 polypeptide comprises cleavage activity associated with an HNH-like domain. In some embodiments, an eaCas9 molecule or eaCas9 polypeptide comprises cleavage activity associated with an N-terminal RuvC-like domain. In some embodiments, an eaCas9 molecule or eaCas9 polypeptide comprises cleavage activity associated with an HNH-like domain and cleavage activity associated with an N-terminal RuvC-like domain. In some embodiments, an eaCas9 molecule or eaCas9 polypeptide comprises an active, or cleavage competent, HNH-like domain and an inactive, or cleavage incompetent, N-terminal RuvC-like domain. In some embodiments, an eaCas9 molecule or eaCas9 polypeptide comprises an inactive, or cleavage incompetent, HNH-like domain and an active, or cleavage competent, N-terminal RuvC-like domain.

Some Cas9 molecules or Cas9 polypeptides have the ability to interact with a gRNA molecule, and in conjunction with the gRNA molecule localize to a core target domain, but are incapable of cleaving the target nucleic acid, or incapable of cleaving at efficient rates. Cas9 molecules having no, or no substantial, cleavage activity are referred to herein as an eiCas9 molecule or eiCas9 polypeptide. For example, an eiCas9 molecule or eiCas9 polypeptide can lack cleavage activity or have substantially less, e.g., less than 20, 10, 5, 1 or 0.1% of the cleavage activity of a reference Cas9 molecule or eiCas9 polypeptide, as measured by an assay described herein.

(g) Targeting and PAMs

A Cas9 molecule or Cas9 polypeptide, is a polypeptide that can interact with a guide RNA (gRNA) molecule and, in concert with the gRNA molecule, localizes to a site which comprises a target domain and a PAM sequence.

In some embodiments, the ability of an eaCas9 molecule or eaCas9 polypeptide to interact with and cleave a target nucleic acid is PAM sequence dependent. A PAM sequence is a sequence in the target nucleic acid. In some embodiments, cleavage of the target nucleic acid occurs upstream from the PAM sequence. EaCas9 molecules from different bacterial species can recognize different sequence motifs (e.g., PAM sequences). In some embodiments, an eaCas9 molecule of S. pyogenes recognizes the sequence motif NGG, NAG, NGA and directs cleavage of a target nucleic acid sequence 1 to 10, e.g., 3 to 5, base pairs upstream from that sequence. See, e.g., Mali et al., Science 2013; 339(6121): 823-826. In some embodiments, an eaCas9 molecule of S. thermophilus recognizes the sequence motif NGGNG and/or NNAGAAW (W=A or T) and directs cleavage of a target nucleic acid sequence 1 to 10, e.g., 3 to 5, base pairs upstream from these sequences. See, e.g., Horvath et al., Science 2010; 327(5962):167-170, and Deveau et al., J Bacteriol 2008; 190(4): 1390-1400. In some embodiments, an eaCas9 molecule of S. mutans recognizes the sequence motif NGG and/or NAAR (R=A or G)) and directs cleavage of a core target nucleic acid sequence 1 to 10, e.g., 3 to 5 base pairs, upstream from this sequence. See, e.g., Deveau et al., J Bacteriol 2008; 190(4): 1390-1400. In some embodiments, an eaCas9 molecule of S. aureus recognizes the sequence motif NNGRR (R=A or G) and directs cleavage of a target nucleic acid sequence 1 to 10, e.g., 3 to 5, base pairs upstream from that sequence. In some embodiments, an eaCas9 molecule of S. aureus recognizes the sequence motif NNGRRT (R=A or G) and directs cleavage of a target nucleic acid sequence 1 to 10, e.g., 3 to 5, base pairs upstream from that sequence. In some embodiments, an eaCas9 molecule of S. aureus recognizes the sequence motif NNGRRV (R=A or G) and directs cleavage of a target nucleic acid sequence 1 to 10, e.g., 3 to 5, base pairs upstream from that sequence. In some embodiments, an eaCas9 molecule of N. meningitidis recognizes the sequence motif NNNNGATT or NNNGCTT (R=A or G, V=A, G or C and directs cleavage of a target nucleic acid sequence 1 to 10, e.g., 3 to 5, base pairs upstream from that sequence. See, e.g., Hou et al., PNAS Early Edition 2013, 1-6. The ability of a Cas9 molecule to recognize a PAM sequence can be determined, e.g., using a transformation assay described in Jinek et al., Science 2012 337:816. In the aforementioned embodiments, N can be any nucleotide residue, e.g., any of A, G, C or T.

As is discussed herein, Cas9 molecules can be engineered to alter the PAM specificity of the Cas9 molecule.

Exemplary naturally occurring Cas9 molecules are described in Chylinski et al., RNA Biology 2013 10:5, 727-737. Such Cas9 molecules include Cas9 molecules of a cluster 1-78 bacterial family.

Exemplary naturally occurring Cas9 molecules include a Cas9 molecule of a cluster 1 bacterial family. Examples include a Cas9 molecule of: S. pyogenes (e.g., strain SF370, MGAS10270, MGAS10750, MGAS2096, MGAS315, MGAS5005, MGAS6180, MGAS9429, NZ131 and SSI-1), S. thermophilus (e.g., strain LMD-9), S. pseudoporcinus (e.g., strain SPIN 20026), S. mutans (e.g., strain UA159, NN2025), S. macacae (e.g., strain NCTC11558), S. gallolyticus (e.g., strain UCN34, ATCC BAA-2069), S. equines (e.g., strain ATCC 9812, MGCS 124), S. dysdalactiae (e.g., strain GGS 124), S. bovis (e.g., strain ATCC 700338), S. anginosus (e.g., strain F0211), S. agalactiae (e.g., strain NEM316, A909), Listeria monocytogenes (e.g., strain F6854), Listeria innocua (L. innocua, e.g., strain Clip11262), Enterococcus italicus (e.g., strain DSM 15952), or Enterococcus faecium (e.g., strain 1,231,408). Another exemplary Cas9 molecule is a Cas9 molecule of Neisseria meningitidis (Hou et al., PNAS Early Edition 2013, 1-6).

In some embodiments, a Cas9 molecule or Cas9 polypeptide, e.g., an eaCas9 molecule or eaCas9 polypeptide, comprises an amino acid sequence: having 60%, 65%, 70%, 75%, 80%, 85%, 90%, 95%, 96%, 97%, 98% or 99% homology with; differs at no more than, 2, 5, 10, 15, 20, 30, or 40% of the amino acid residues when compared with; differs by at least 1, 2, 5, 10 or 20 amino acids but by no more than 100, 80, 70, 60, 50, 40 or 30 amino acids from; or is identical to any Cas9 molecule sequence described herein, or a naturally occurring Cas9 molecule sequence, e.g., a Cas9 molecule from a species listed herein (e.g., SEQ ID NOS:112-115) or described in Chylinski et al., RNA Biology 2013 10:5, 727-737; Hou et al., PNAS Early Edition 2013, 1-6. In some embodiments, the Cas9 molecule or Cas9 polypeptide comprises one or more of the following activities: a nickase activity; a double stranded cleavage activity (e.g., an endonuclease and/or exonuclease activity); a helicase activity; or the ability, together with a gRNA molecule, to home to a target nucleic acid.

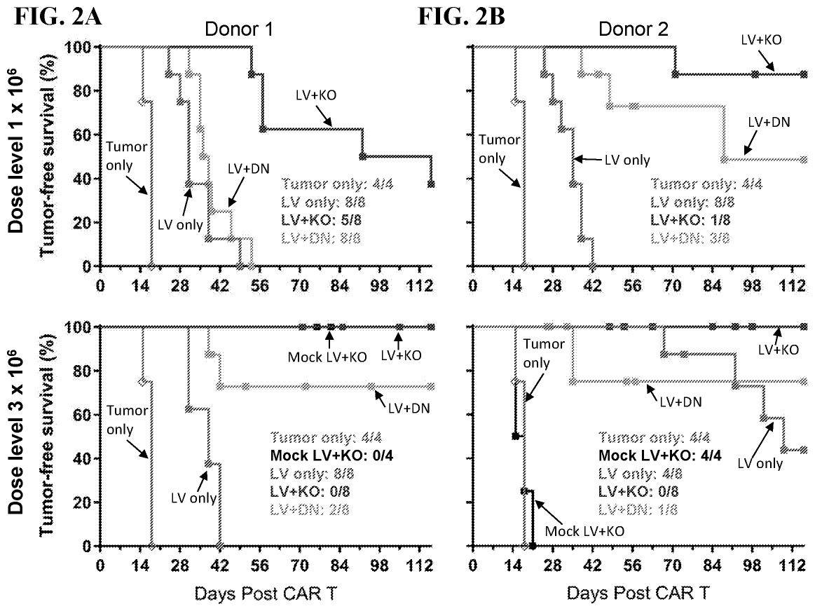

In some embodiments, a Cas9 molecule or Cas9 polypeptide comprises the amino acid sequence of the consensus sequence of WO2015/161276, e.g., in FIGS. 2A-2G therein, wherein “*” indicates any amino acid found in the corresponding position in the amino acid sequence of a Cas9 molecule of S. pyogenes, S. thermophilus, S. mutans and L. innocua, and “-” indicates any amino acid. In some embodiments, a Cas9 molecule or Cas9 polypeptide differs from the sequence of the consensus sequence of SEQ ID NOS:112-117 or the consensus sequence disclosed in WO2015/161276, e.g., in FIGS. 2A-2G therein by at least 1, but no more than 2, 3, 4, 5, 6, 7, 8, 9, or 10 amino acid residues. In some embodiments, a Cas9 molecule or Cas9 polypeptide comprises the amino acid sequence of SEQ ID NO:117 or as described in WO2015/161276, e.g., in FIGS. 7A-7B therein, wherein “*” indicates any amino acid found in the corresponding position in the amino acid sequence of a Cas9 molecule of S. pyogenes, or N. meningitidis, “-” indicates any amino acid, and “-” indicates any amino acid or absent.

In some embodiments, a Cas9 molecule or Cas9 polypeptide differs from the sequence of SEQ ID NO:116 or 117 or as described in WO2015/161276, e.g., in FIGS. 7A-7B therein by at least 1, but no more than 2, 3, 4, 5, 6, 7, 8, 9, or 10 amino acid residues.

A comparison of the sequence of a number of Cas9 molecules indicate that certain regions are conserved. These are identified as: region 1 (residues 1 to 180, or in the case of region 1′ residues 120 to 180); region 2 (residues 360 to 480); region 3 (residues 660 to 720); region 4 (residues 817 to 900); and region 5 (residues 900 to 960).

In some embodiments, a Cas9 molecule or Cas9 polypeptide comprises regions 1-5, together with sufficient additional Cas9 molecule sequence to provide a biologically active molecule, e.g., a Cas9 molecule having at least one activity described herein. In some embodiments, each of regions 1-6, independently, have, 50%, 60%, 70%, or 80% homology with the corresponding residues of a Cas9 molecule or Cas9 polypeptide described herein, e.g., set forth in SEQ ID NOS:112-117 or a sequence disclosed in WO2015/161276, e.g., from FIGS. 2A-2G or from FIGS. 7A-7B therein.

In some embodiments, a Cas9 molecule or Cas9 polypeptide, e.g., an eaCas9 molecule or eaCas9 polypeptide, comprises an amino acid sequence referred to as region 1, having 50%, 60%, 70%, 80%, 85%, 90%, 95%, 96%, 97%, 98% or 99% homology with amino acids 1-180 (the numbering is according to the motif sequence in FIGS. 2A-2G of WO 2015/161276; 52% of residues in the four Cas9 sequences in FIGS. 2A-2G of WO 2015/161276 are conserved) of the amino acid sequence of Cas9 of S. pyogenes; differs by at least 1, 2, 5, 10 or 20 amino acids but by no more than 90, 80, 70, 60, 50, 40 or 30 amino acids from amino acids 1-180 of the amino acid sequence of Cas9 of S. pyogenes, S. thermophilus, S. mutans or L. innocua; or, is identical to 1-180 of the amino acid sequence of Cas9 of S. pyogenes, S. thermophilus, S. mutans or L. innocua.

In some embodiments, a Cas9 molecule or Cas9 polypeptide, e.g., an eaCas9 molecule or eaCas9 polypeptide, comprises an amino acid sequence referred to as region 1′, having 55%, 60%, 65%, 70%, 75%, 80%, 85%, 90%, 95%, 96%, 97%, 98% or 99% homology with amino acids 120-180 (55% of residues in the four Cas9 sequences in FIGS. 2A-2G of WO 2015/161276 are conserved) of the amino acid sequence of Cas9 of S. pyogenes, S. thermophilus, S. mutans or L. innocua; differs by at least 1, 2, or 5 amino acids but by no more than 35, 30, 25, 20 or 10 amino acids from amino acids 120-180 of the amino acid sequence of Cas9 of S. pyogenes, S. thermophilus, S. mutans or L. innocua; or, is identical to 120-180 of the amino acid sequence of Cas9 of S. pyogenes, S. thermophilus, S. mutans or L. innocua.

In some embodiments, a Cas9 molecule or Cas9 polypeptide, e.g., an eaCas9 molecule or eaCas9 polypeptide, comprises an amino acid sequence referred to as region 2, having 50%, 55%, 60%, 65%, 70%, 75%, 80%, 85%, 90%, 95%, 96%, 97%, 98% or 99% homology with amino acids 360-480 (52% of residues in the four Cas9 sequences in FIGS. 2A-2G of WO 2015/161276 are conserved) of the amino acid sequence of Cas9 of S. pyogenes, S. thermophilus, S. mutans or L. innocua; differs by at least 1, 2, or 5 amino acids but by no more than 35, 30, 25, 20 or 10 amino acids from amino acids 360-480 of the amino acid sequence of Cas9 of S. pyogenes, S. thermophilus, S. mutans or L. innocua; or, is identical to 360-480 of the amino acid sequence of Cas9 of S. pyogenes, S. thermophilus, S. mutans or L. innocua.

In some embodiments, a Cas9 molecule or Cas9 polypeptide, e.g., an eaCas9 molecule or eaCas9 polypeptide, comprises an amino acid sequence referred to as region 3, having 55%, 60%, 65%, 70%, 75%, 80%, 85%, 90%, 95%, 96%, 97%, 98%, or 99% homology with amino acids 660-720 (56% of residues in the four Cas9 sequences in FIGS. 2A-2G of WO 2015/161276 are conserved) of the amino acid sequence of Cas9 of S. pyogenes, S. thermophilus, S. mutans or L. innocua; differs by at least 1, 2, or 5 amino acids but by no more than 35, 30, 25, 20 or 10 amino acids from amino acids 660-720 of the amino acid sequence of Cas9 of S. pyogenes, S. thermophilus, S. mutans or L. innocua; or, is identical to 660-720 of the amino acid sequence of Cas9 of S. pyogenes, S. thermophilus, S. mutans or L. innocua.

In some embodiments, a Cas9 molecule or Cas9 polypeptide, e.g., an eaCas9 molecule or eaCas9 polypeptide, comprises an amino acid sequence referred to as region 4, having 50%, 55%, 60%, 65%, 70%, 75%, 80%, 85%, 90%, 95%, 96%, 97%, 98%, or 99% homology with amino acids 817-900 (55% of residues in the four Cas9 sequences in FIGS. 2A-2G of WO 2015/161276 are conserved) of the amino acid sequence of Cas9 of S. pyogenes, S. thermophilus, S. mutans or L. innocua; differs by at least 1, 2, or 5 amino acids but by no more than 35, 30, 25, 20 or 10 amino acids from amino acids 817-900 of the amino acid sequence of Cas9 of S. pyogenes, S. thermophilus, S. mutans or L. innocua; or, is identical to 817-900 of the amino acid sequence of Cas9 of S. pyogenes, S. thermophilus, S. mutans or L. innocua.

In some embodiments, a Cas9 molecule or Cas9 polypeptide, e.g., an eaCas9 molecule or eaCas9 polypeptide, comprises an amino acid sequence referred to as region 5, having 50%, 55%, 60%, 65%, 70%, 75%, 80%, 85%, 90%, 95%, 96%, 97%, 98%, or 99% homology with amino acids 900-960 (60% of residues in the four Cas9 sequences in FIGS. 2A-2G of WO 2015/161276 are conserved) of the amino acid sequence of Cas9 of S. pyogenes, S. thermophilus, S. mutans or L. innocua; differs by at least 1, 2, or 5 amino acids but by no more than 35, 30, 25, 20 or 10 amino acids from amino acids 900-960 of the amino acid sequence of Cas9 of S. pyogenes, S. thermophilus, S. mutans or L. innocua; or, is identical to 900-960 of the amino acid sequence of Cas9 of S. pyogenes, S. thermophilus, S. mutans or L. innocua.

(h) Engineered or Altered Cas9 Molecules and Cas9 Polypeptides

Cas9 molecules and Cas9 polypeptides described herein, e.g., naturally occurring Cas9 molecules, can possess any of a number of properties, including: nickase activity, nuclease activity (e.g., endonuclease and/or exonuclease activity); helicase activity; the ability to associate functionally with a gRNA molecule; and the ability to target (or localize to) a site on a nucleic acid (e.g., PAM recognition and specificity). In some embodiments, a Cas9 molecule or Cas9 polypeptide can include all or a subset of these properties. In typical embodiments, a Cas9 molecule or Cas9 polypeptide has the ability to interact with a gRNA molecule and, in concert with the gRNA molecule, localize to a site in a nucleic acid. Other activities, e.g., PAM specificity, cleavage activity, or helicase activity can vary more widely in Cas9 molecules and Cas9 polypeptides.

Cas9 molecules include engineered Cas9 molecules and engineered Cas9 polypeptides (“engineered,” as used in this context, means merely that the Cas9 molecule or Cas9 polypeptide differs from a reference sequences, and implies no process or origin limitation). An engineered Cas9 molecule or Cas9 polypeptide can comprise altered enzymatic properties, e.g., altered nuclease activity, (as compared with a naturally occurring or other reference Cas9 molecule) or altered helicase activity. As discussed herein, an engineered Cas9 molecule or Cas9 polypeptide can have nickase activity (as opposed to double strand nuclease activity). In some embodiments an engineered Cas9 molecule or Cas9 polypeptide can have an alteration that alters its size, e.g., a deletion of amino acid sequence that reduces its size, e.g., without significant effect on one or more, or any Cas9 activity. In some embodiments, an engineered Cas9 molecule or Cas9 polypeptide can comprise an alteration that affects PAM recognition. E.g., an engineered Cas9 molecule can be altered to recognize a PAM sequence other than that recognized by the endogenous wild-type PI domain. In some embodiments a Cas9 molecule or Cas9 polypeptide can differ in sequence from a naturally occurring Cas9 molecule but not have significant alteration in one or more Cas9 activities.

Cas9 molecules or Cas9 polypeptides with desired properties can be made in a number of ways, e.g., by alteration of a parental, e.g., naturally occurring, Cas9 molecules or Cas9 polypeptides, to provide an altered Cas9 molecule or Cas9 polypeptide having a desired property. For example, one or more mutations or differences relative to a parental Cas9 molecule, e.g., a naturally occurring or engineered Cas9 molecule, can be introduced. Such mutations and differences comprise: substitutions (e.g., conservative substitutions or substitutions of non-essential amino acids); insertions; or deletions. In some embodiments, a Cas9 molecule or Cas9 polypeptide can comprises one or more mutations or differences, e.g., at least 1, 2, 3, 4, 5, 10, 15, 20, 30, 40 or 50 mutations but less than 200, 100, or 80 mutations relative to a reference, e.g., a parental, Cas9 molecule.

In some embodiments, a mutation or mutations do not have a substantial effect on a Cas9 activity, e.g. a Cas9 activity described herein. In some embodiments, a mutation or mutations have a substantial effect on a Cas9 activity, e.g. a Cas9 activity described herein.

(i) Non-Cleaving and Modified-Cleavage Cas9 Molecules and Cas9 Polypeptides

In some embodiments, a Cas9 molecule or Cas9 polypeptide comprises a cleavage property that differs from naturally occurring Cas9 molecules, e.g., that differs from the naturally occurring Cas9 molecule having the closest homology. For example, a Cas9 molecule or Cas9 polypeptide can differ from naturally occurring Cas9 molecules, e.g., a Cas9 molecule of S. pyogenes, as follows: its ability to modulate, e.g., decreased or increased, cleavage of a double stranded nucleic acid (endonuclease and/or exonuclease activity), e.g., as compared to a naturally occurring Cas9 molecule (e.g., a Cas9 molecule of S. pyogenes); its ability to modulate, e.g., decreased or increased, cleavage of a single strand of a nucleic acid, e.g., a non-complementary strand of a nucleic acid molecule or a complementary strand of a nucleic acid molecule (nickase activity), e.g., as compared to a naturally occurring Cas9 molecule (e.g., a Cas9 molecule of S. pyogenes); or the ability to cleave a nucleic acid molecule, e.g., a double stranded or single stranded nucleic acid molecule, can be eliminated.

(j) Modified Cleavage eaCas9 Molecules and eaCas9 Polypeptides

In some embodiments, an eaCas9 molecule or eaCas9 polypeptide comprises one or more of the following activities: cleavage activity associated with an N-terminal RuvC-like domain; cleavage activity associated with an HNH-like domain; cleavage activity associated with an HNH-like domain and cleavage activity associated with an N-terminal RuvC-like domain.

In some embodiments, an eaCas9 molecule or eaCas9 polypeptide comprises an active, or cleavage competent, HNH-like domain and an inactive, or cleavage incompetent, N-terminal RuvC-like domain. An exemplary inactive, or cleavage incompetent N-terminal RuvC-like domain can have a mutation of an aspartic acid in an N-terminal RuvC-like domain, e.g., an aspartic acid at position 9 of the consensus sequence of SEQ ID NOS:112-117 or the consensus sequence disclosed in WO2015/161276, e.g., in FIGS. 2A-2G therein or an aspartic acid at position 10 of SEQ ID NO:117, e.g., can be substituted with an alanine. In some embodiments, the eaCas9 molecule or eaCas9 polypeptide differs from wild type in the N-terminal RuvC-like domain and does not cleave the target nucleic acid, or cleaves with significantly less efficiency, e.g., less than 20, 10, 5, 1 or 0.1% of the cleavage activity of a reference Cas9 molecule, e.g., as measured by an assay described herein. The reference Cas9 molecule can by a naturally occurring unmodified Cas9 molecule, e.g., a naturally occurring Cas9 molecule such as a Cas9 molecule of S. pyogenes, or S. thermophilus. In some embodiments, the reference Cas9 molecule is the naturally occurring Cas9 molecule having the closest sequence identity or homology.

In some embodiments, an eaCas9 molecule or eaCas9 polypeptide comprises an inactive, or cleavage incompetent, HNH domain and an active, or cleavage competent, N-terminal RuvC-like domain. Exemplary inactive, or cleavage incompetent HNH-like domains can have a mutation at one or more of: a histidine in an HNH-like domain, e.g., a histidine shown at position 856 of the consensus sequence of SEQ ID NOS:112-117 or the consensus sequence disclosed in WO2015/161276, e.g., in FIGS. 2A-2G therein, e.g., can be substituted with an alanine; and one or more asparagines in an HNH-like domain, e.g., an asparagine shown at position 870 of the consensus sequence of SEQ ID NOS:112-117 or the consensus sequence disclosed in WO2015/161276, e.g., in FIGS. 2A-2G therein and/or at position 879 of the consensus sequence of SEQ ID NOS:112-117 or the consensus sequence disclosed in WO2015/161276, e.g., in FIGS. 2A-2G therein, e.g., can be substituted with an alanine. In some embodiments, the eaCas9 differs from wild type in the HNH-like domain and does not cleave the target nucleic acid, or cleaves with significantly less efficiency, e.g., less than 20, 10, 5, 1 or 0.1% of the cleavage activity of a reference Cas9 molecule, e.g., as measured by an assay described herein. The reference Cas9 molecule can by a naturally occurring unmodified Cas9 molecule, e.g., a naturally occurring Cas9 molecule such as a Cas9 molecule of S. pyogenes, or S. thermophilus. In some embodiments, the reference Cas9 molecule is the naturally occurring Cas9 molecule having the closest sequence identity or homology.

In some embodiments, an eaCas9 molecule or eaCas9 polypeptide comprises an inactive, or cleavage incompetent, HNH domain and an active, or cleavage competent, N-terminal RuvC-like domain. Exemplary inactive, or cleavage incompetent HNH-like domains can have a mutation at one or more of: a histidine in an HNH-like domain, e.g., a histidine shown at position 856 of the consensus sequence of SEQ ID NOS:112-117 or the consensus sequence disclosed in WO2015/161276, e.g., in FIGS. 2A-2G therein, e.g., can be substituted with an alanine; and one or more asparagines in an HNH-like domain, e.g., an asparagine shown at position 870 of the consensus sequence of SEQ ID NOS:112-117 or the consensus sequence disclosed in WO2015/161276, e.g., in FIGS. 2A-2G therein and/or at position 879 of the consensus sequence of SEQ ID NOS:112-117 or the consensus sequence disclosed in WO2015/161276, e.g., in FIGS. 2A-2G therein, e.g., can be substituted with an alanine. In some embodiments, the eaCas9 differs from wild type in the HNH-like domain and does not cleave the target nucleic acid, or cleaves with significantly less efficiency, e.g., less than 20, 10, 5, 1 or 0.1% of the cleavage activity of a reference Cas9 molecule, e.g., as measured by an assay described herein. The reference Cas9 molecule can by a naturally occurring unmodified Cas9 molecule, e.g., a naturally occurring Cas9 molecule such as a Cas9 molecule of S. pyogenes, or S. thermophilus. In some embodiments, the reference Cas9 molecule is the naturally occurring Cas9 molecule having the closest sequence identity or homology.

(k) Alterations in the Ability to Cleave One or Both Strands of a Target Nucleic Acid

In some embodiments, exemplary Cas9 activities comprise one or more of PAM specificity, cleavage activity, and helicase activity. A mutation(s) can be present, e.g., in: one or more RuvC-like domain, e.g., an N-terminal RuvC-like domain; an HNH-like domain; a region outside the RuvC-like domains and the HNH-like domain. In some embodiments, a mutation(s) is present in a RuvC-like domain, e.g., an N-terminal RuvC-like. In some embodiments, a mutation(s) is present in an HNH-like domain. In some embodiments, mutations are present in both a RuvC-like domain, e.g., an N-terminal RuvC-like domain, and an HNH-like domain.

Exemplary mutations that may be made in the RuvC domain or HNH domain with reference to the S. pyogenes sequence include: D10A, E762A, H840A, N854A, N863A and/or D986A.

In some embodiments, a Cas9 molecule or Cas9 polypeptide is an eiCas9 molecule or eiCas9 polypeptide comprising one or more differences in a RuvC domain and/or in an HNH domain as compared to a reference Cas9 molecule, and the eiCas9 molecule or eiCas9 polypeptide does not cleave a nucleic acid, or cleaves with significantly less efficiency than does wild type, e.g., when compared with wild type in a cleavage assay, e.g., as described herein, cuts with less than 50, 25, 10, or 1% of a reference Cas9 molecule, as measured by an assay described herein.

Whether or not a particular sequence, e.g., a substitution, may affect one or more activity, such as targeting activity, cleavage activity, etc., can be evaluated or predicted, e.g., by evaluating whether the mutation is conservative. In some embodiments, a “non-essential” amino acid residue, as used in the context of a Cas9 molecule, is a residue that can be altered from the wild-type sequence of a Cas9 molecule, e.g., a naturally occurring Cas9 molecule, e.g., an eaCas9 molecule, without abolishing or more preferably, without substantially altering a Cas9 activity (e.g., cleavage activity), whereas changing an “essential” amino acid residue results in a substantial loss of activity (e.g., cleavage activity).

In some embodiments, a Cas9 molecule or Cas9 polypeptide comprises a cleavage property that differs from naturally occurring Cas9 molecules, e.g., that differs from the naturally occurring Cas9 molecule having the closest homology. For example, a Cas9 molecule or Cas9 polypeptide can differ from naturally occurring Cas9 molecules, e.g., a Cas9 molecule of S. aureus, S. pyogenes, or C. jejuni as follows: its ability to modulate, e.g., decreased or increased, cleavage of a double stranded break (endonuclease and/or exonuclease activity), e.g., as compared to a naturally occurring Cas9 molecule (e.g., a Cas9 molecule of S. aureus, S. pyogenes, or C. jejuni); its ability to modulate, e.g., decreased or increased, cleavage of a single strand of a nucleic acid, e.g., a non-complementary strand of a nucleic acid molecule or a complementary strand of a nucleic acid molecule (nickase activity), e.g., as compared to a naturally occurring Cas9 molecule (e.g., a Cas9 molecule of S. aureus, S. pyogenes, or C. jejuni); or the ability to cleave a nucleic acid molecule, e.g., a double stranded or single stranded nucleic acid molecule, can be eliminated.

In some embodiments, the altered Cas9 molecule or Cas9 polypeptide is an eaCas9 molecule or eaCas9 polypeptide comprising one or more of the following activities: cleavage activity associated with a RuvC domain; cleavage activity associated with an HNH domain; cleavage activity associated with an HNH domain and cleavage activity associated with a RuvC domain.

In some embodiments, the altered Cas9 molecule or Cas9 polypeptide is an eiCas9 molecule or eaCas9 polypeptide which does not cleave a nucleic acid molecule (either double stranded or single stranded nucleic acid molecules) or cleaves a nucleic acid molecule with significantly less efficiency, e.g., less than 20, 10, 5, 1 or 0.1% of the cleavage activity of a reference Cas9 molecule, e.g., as measured by an assay described herein. The reference Cas9 molecule can be a naturally occurring unmodified Cas9 molecule, e.g., a naturally occurring Cas9 molecule such as a Cas9 molecule of S. pyogenes, S. thermophilus, S. aureus, C. jejuni or N. meningitidis. In some embodiments, the reference Cas9 molecule is the naturally occurring Cas9 molecule having the closest sequence identity or homology. In some embodiments, the eiCas9 molecule or eiCas9 polypeptide lacks substantial cleavage activity associated with a RuvC domain and cleavage activity associated with an HNH domain.

In some embodiments, the altered Cas9 molecule or Cas9 polypeptide is an eaCas9 molecule or eaCas9 polypeptide comprising the fixed amino acid residues of S. pyogenes shown in the consensus sequence disclosed in WO2015/161276, e.g., in FIGS. 2A-2G therein, and has one or more amino acids that differ from the amino acid sequence of S. pyogenes (e.g., has a substitution) at one or more residue (e.g., 2, 3, 5, 10, 15, 20, 30, 50, 70, 80, 90, 100, 200 amino acid residues) in SEQ ID NO:117 or residue represented by an “-” in the consensus sequence disclosed in WO2015/161276, e.g., in FIGS. 2A-2G therein.

In some embodiments, the altered Cas9 molecule or Cas9 polypeptide comprises a sequence in which: the sequence corresponding to the fixed sequence of the consensus sequence disclosed in FIGS. 2A-2G of WO2015/161276 differs at no more than 1, 2, 3, 4, 5, 10, 15, or 20% of the fixed residues in the consensus sequence disclosed in FIGS. 2A-2G of WO2015/161276, the sequence corresponding to the residues identified by “*” in the consensus sequence disclosed in FIGS. 2A-2G of WO2015/161276 differ at no more than 1, 2, 3, 4, 5, 10, 15, 20, 25, 30, 35, or 40% of the “*” residues from the corresponding sequence of naturally occurring Cas9 molecule, e.g., an S. pyogenes Cas9 molecule; and, the sequence corresponding to the residues identified by “-” in the consensus sequence disclosed in FIGS. 2A-2G of WO2015/161276 differ at no more than 5, 10, 15, 20, 25, 30, 35, 40, 45, 55, or 60% of the “-” residues from the corresponding sequence of naturally occurring Cas9 molecule, e.g., an S. pyogenes Cas9 molecule.

In some embodiments, the altered Cas9 molecule or Cas9 polypeptide is an eaCas9 molecule or eaCas9 polypeptide comprising the fixed amino acid residues of S. thermophilus shown in the consensus sequence disclosed in FIGS. 2A-2G of WO2015/161276, and has one or more amino acids that differ from the amino acid sequence of S. thermophilus (e.g., has a substitution) at one or more residue (e.g., 2, 3, 5, 10, 15, 20, 30, 50, 70, 80, 90, 100, 200 amino acid residues) represented by an “-” in the consensus sequence disclosed in FIGS. 2A-2G of WO2015/161276.

In some embodiments the altered Cas9 molecule or Cas9 polypeptide comprises a sequence in which: the sequence corresponding to the fixed sequence of the consensus sequence disclosed in FIGS. 2A-2G of WO2015/161276 differs at no more than 1, 2, 3, 4, 5, 10, 15, or 20% of the fixed residues in the consensus sequence disclosed in FIGS. 2A-2G of WO2015/161276, the sequence corresponding to the residues identified by “*” in the consensus sequence disclosed in FIGS. 2A-2G of WO2015/161276 differ at no more than 1, 2, 3, 4, 5, 10, 15, 20, 25, 30, 35, or 40% of the “*” residues from the corresponding sequence of naturally occurring Cas9 molecule, e.g., an S. thermophilus Cas9 molecule; and the sequence corresponding to the residues identified by “-” in the consensus sequence disclosed in FIGS. 2A-2G of WO2015/161276 differ at no more than 5, 10, 15, 20, 25, 30, 35, 40, 45, 55, or 60% of the “-” residues from the corresponding sequence of naturally occurring Cas9 molecule, e.g., an S. thermophilus Cas9 molecule.

In some embodiments, the altered Cas9 molecule or Cas9 polypeptide is an eaCas9 molecule or eaCas9 polypeptide comprising the fixed amino acid residues of S. mutans shown in the consensus sequence disclosed in FIGS. 2A-2G of WO2015/161276, and has one or more amino acids that differ from the amino acid sequence of S. mutans (e.g., has a substitution) at one or more residue (e.g., 2, 3, 5, 10, 15, 20, 30, 50, 70, 80, 90, 100, 200 amino acid residues) represented by an “-” in the consensus sequence disclosed in FIGS. 2A-2G of WO2015/161276.

In some embodiments, the altered Cas9 molecule or Cas9 polypeptide comprises a sequence in which: the sequence corresponding to the fixed sequence of the consensus sequence disclosed in FIGS. 2A-2G of WO2015/161276 differs at no more than 1, 2, 3, 4, 5, 10, 15, or 20% of the fixed residues in the consensus sequence disclosed in FIGS. 2A-2G of WO2015/161276, the sequence corresponding to the residues identified by “*” in the consensus sequence disclosed in FIGS. 2A-2G of WO2015/161276 differ at no more than 1, 2, 3, 4, 5, 10, 15, 20, 25, 30, 35, or 40% of the “*” residues from the corresponding sequence of naturally occurring Cas9 molecule, e.g., an S. mutans Cas9 molecule; and, the sequence corresponding to the residues identified by “-” in the consensus sequence disclosed in FIGS. 2A-2G of WO2015/161276 differ at no more than 5, 10, 15, 20, 25, 30, 35, 40, 45, 55, or 60% of the “-” residues from the corresponding sequence of naturally occurring Cas9 molecule, e.g., an S. mutans Cas9 molecule.

In some embodiments, the altered Cas9 molecule or Cas9 polypeptide is an eaCas9 molecule or eaCas9 polypeptide comprising the fixed amino acid residues of L. innocula shown in the consensus sequence disclosed in FIGS. 2A-2G of WO2015/161276, and has one or more amino acids that differ from the amino acid sequence of L. innocula (e.g., has a substitution) at one or more residue (e.g., 2, 3, 5, 10, 15, 20, 30, 50, 70, 80, 90, 100, 200 amino acid residues) represented by an “-” in the consensus sequence disclosed in FIGS. 2A-2G of WO2015/161276. In some embodiments, the altered Cas9 molecule or Cas9 polypeptide comprises a sequence in which: the sequence corresponding to the fixed sequence of the consensus sequence disclosed in FIGS. 2A-2G of WO2015/161276 differs at no more than 1, 2, 3, 4, 5, 10, 15, or 20% of the fixed residues in the consensus sequence disclosed in FIGS. 2A-2G of WO2015/161276, the sequence corresponding to the residues identified by “*” in the consensus sequence disclosed in FIGS. 2A-2G of WO2015/161276 differ at no more than 1, 2, 3, 4, 5, 10, 15, 20, 25, 30, 35, or 40% of the “*” residues from the corresponding sequence of naturally occurring Cas9 molecule, e.g., an L. innocula Cas9 molecule; and, the sequence corresponding to the residues identified by “-” in the consensus sequence disclosed in FIGS. 2A-2G of WO2015/161276 differ at no more than 5, 10, 15, 20, 25, 30, 35, 40, 45, 55, or 60% of the “-” residues from the corresponding sequence of naturally occurring Cas9 molecule, e.g., an L. innocula Cas9 molecule.

In some embodiments, the altered Cas9 molecule or Cas9 polypeptide, e.g., an eaCas9 molecule, can be a fusion, e.g., of two of more different Cas9 molecules or Cas9 polypeptides, e.g., of two or more naturally occurring Cas9 molecules of different species. For example, a fragment of a naturally occurring Cas9 molecule of one species can be fused to a fragment of a Cas9 molecule of a second species. As an example, a fragment of Cas9 molecule of S. pyogenes comprising an N-terminal RuvC-like domain can be fused to a fragment of Cas9 molecule of a species other than S. pyogenes (e.g., S. thermophilus) comprising an HNH-like domain.

(l) Cas9 Molecules with Altered PAM Recognition or No PAM Recognition

Naturally occurring Cas9 molecules can recognize specific PAM sequences, for example the PAM recognition sequences described herein for, e.g., S. pyogenes, S. thermophilus, S. mutans, S. aureus and N. meningitidis.

In some embodiments, a Cas9 molecule or Cas9 polypeptide has the same PAM specificities as a naturally occurring Cas9 molecule. In other embodiments, a Cas9 molecule or Cas9 polypeptide has a PAM specificity not associated with a naturally occurring Cas9 molecule, or a PAM specificity not associated with the naturally occurring Cas9 molecule to which it has the closest sequence homology. For example, a naturally occurring Cas9 molecule can be altered, e.g., to alter PAM recognition, e.g., to alter the PAM sequence that the Cas9 molecule or Cas9 polypeptide recognizes to decrease off target sites and/or improve specificity; or eliminate a PAM recognition requirement. In some embodiments, a Cas9 molecule can be altered, e.g., to increase length of PAM recognition sequence and/or improve Cas9 specificity to high level of identity, e.g., to decrease off target sites and increase specificity. In some embodiments, the length of the PAM recognition sequence is at least 4, 5, 6, 7, 8, 9, 10 or 15 amino acids in length.

Cas9 molecules or Cas9 polypeptides that recognize different PAM sequences and/or have reduced off-target activity can be generated using directed evolution. Exemplary methods and systems that can be used for directed evolution of Cas9 molecules are described, e.g., in Esvelt et al. Nature 2011, 472(7344): 499-503. Candidate Cas9 molecules can be evaluated, e.g., by methods described herein.

Alterations of the PI domain, which mediates PAM recognition, are discussed herein.

(m) Synthetic Cas9 Molecules and Cas9 Polypeptides with Altered PI Domains

Current genome-editing methods are limited in the diversity of target sequences that can be targeted by the PAM sequence that is recognized by the Cas9 molecule utilized. A synthetic Cas9 molecule (or Syn-Cas9 molecule), or synthetic Cas9 polypeptide (or Syn-Cas9 polypeptide), as that term is used herein, refers to a Cas9 molecule or Cas9 polypeptide that comprises a Cas9 core domain from one bacterial species and a functional altered PI domain, i.e., a PI domain other than that naturally associated with the Cas9 core domain, e.g., from a different bacterial species.

In some embodiments, the altered PI domain recognizes a PAM sequence that is different from the PAM sequence recognized by the naturally-occurring Cas9 from which the Cas9 core domain is derived. In some embodiments, the altered PI domain recognizes the same PAM sequence recognized by the naturally-occurring Cas9 from which the Cas9 core domain is derived, but with different affinity or specificity. A Syn-Cas9 molecule or Syn-Cas9 polypeptide can be, respectively, a Syn-eaCas9 molecule or Syn-eaCas9 polypeptide or a Syn-eiCas9 molecule Syn-eiCas9 polypeptide.

An exemplary Syn-Cas9 molecule or Syn-Cas9 polypeptide comprises: a) a Cas9 core domain, e.g., a Cas9 core domain, e.g., a S. aureus, S. pyogenes, or C. jejuni Cas9 core domain; and b) an altered PI domain from a species X Cas9 sequence.

In some embodiments, the RKR motif (the PAM binding motif) of said altered PI domain comprises: differences at 1, 2, or 3 amino acid residues; a difference in amino acid sequence at the first, second, or third position; differences in amino acid sequence at the first and second positions, the first and third positions, or the second and third positions; as compared with the sequence of the RKR motif of the native or endogenous PI domain associated with the Cas9 core domain.

In some embodiments, a Syn-Cas9 molecule or Syn-Cas9 polypeptide may also be size-optimized, e.g., the Syn-Cas9 molecule or Syn-Cas9 polypeptide comprises one or more deletions, and optionally one or more linkers disposed between the amino acid residues flanking the deletions. In some embodiments, a Syn-Cas9 molecule or Syn-Cas9 polypeptide comprises a REC deletion.

(n) Size-Optimized Cas9 Molecules and Cas9 Polypeptides

Engineered Cas9 molecules and engineered Cas9 polypeptides described herein include a Cas9 molecule or Cas9 polypeptide comprising a deletion that reduces the size of the molecule while still retaining desired Cas9 properties, e.g., essentially native conformation, Cas9 nuclease activity, and/or target nucleic acid molecule recognition. The Cas9 molecules or Cas9 polypeptides used in the context of the provided embodiments can comprise one or more deletions and optionally one or more linkers, wherein a linker is disposed between the amino acid residues that flank the deletion.

A Cas9 molecule, e.g., a S. aureus, S. pyogenes, or C. jejuni, Cas9 molecule, having a deletion is smaller, e.g., has reduced number of amino acids, than the corresponding naturally-occurring Cas9 molecule. The smaller size of the Cas9 molecules allows increased flexibility for delivery methods, and thereby increases utility for genome-editing. A Cas9 molecule or Cas9 polypeptide can comprise one or more deletions that do not substantially affect or decrease the activity of the resultant Cas9 molecules or Cas9 polypeptides described herein. Activities that are retained in the Cas9 molecules or Cas9 polypeptides comprising a deletion as described herein include one or more of the following: a nickase activity, i.e., the ability to cleave a single strand, e.g., the non-complementary strand or the complementary strand, of a nucleic acid molecule; a double stranded nuclease activity, i.e., the ability to cleave both strands of a double stranded nucleic acid and create a double stranded break, which In some embodiments is the presence of two nickase activities; an endonuclease activity; an exonuclease activity; a helicase activity, i.e., the ability to unwind the helical structure of a double stranded nucleic acid; and recognition activity of a nucleic acid molecule, e.g., a target nucleic acid or a gRNA.

Activity of the Cas9 molecules or Cas9 polypeptides described herein can be assessed using the activity assays described herein or are known.

(o) Identifying Regions Suitable for Deletion

Suitable regions of Cas9 molecules for deletion can be identified by a variety of methods. Naturally-occurring orthologous Cas9 molecules from various bacterial species, can be modeled onto the crystal structure of S. pyogenes Cas9 (Nishimasu et al., Cell, 156:935-949, 2014) to examine the level of conservation across the selected Cas9 orthologs with respect to the three-dimensional conformation of the protein. Less conserved or unconserved regions that are spatially located distant from regions involved in Cas9 activity, e.g., interface with the target nucleic acid molecule and/or gRNA, represent regions or domains are candidates for deletion without substantially affecting or decreasing Cas9 activity.

(p) REC-Optimized Cas9 Molecules and Cas9 Polypeptides

A REC-optimized Cas9 molecule, or a REC-optimized Cas9 polypeptide, as that term is used herein, refers to a Cas9 molecule or Cas9 polypeptide that comprises a deletion in one or both of the REC2 domain and the RE1_{CT} domain (collectively a REC deletion), wherein the deletion comprises at least 10% of the amino acid residues in the cognate domain. A REC-optimized Cas9 molecule or Cas9 polypeptide can be an eaCas9 molecule or eaCas9 polypeptide, or an eiCas9 molecule or eiCas9 polypeptide. An exemplary REC-optimized Cas9 molecule or REC-optimized Cas9 polypeptide comprises: a) a deletion selected from: i) a REC2 deletion; ii) a REC1_{CT} deletion; or iii) a REC1_{SUB} deletion.

Optionally, a linker is disposed between the amino acid residues that flank the deletion. In some embodiments a Cas9 molecule or Cas9 polypeptide includes only one deletion, or only two deletions. A Cas9 molecule or Cas9 polypeptide can comprise a REC2 deletion and a REC1_{CT} deletion. A Cas9 molecule or Cas9 polypeptide can comprise a REC2 deletion and a REC1_{SUB} deletion.

Generally, the deletion will contain at least 10% of the amino acids in the cognate domain, e.g., a REC2 deletion will include at least 10% of the amino acids in the REC2 domain. A deletion can comprise: at least 10, 20, 30, 40, 50, 60, 70, 80, or 90% of the amino acid residues of its cognate domain; all of the amino acid residues of its cognate domain; an amino acid residue outside its cognate domain; a plurality of amino acid residues outside its cognate domain; the amino acid residue immediately N terminal to its cognate domain; the amino acid residue immediately C terminal to its cognate domain; the amino acid residue immediately N terminal to its cognate and the amino acid residue immediately C terminal to its cognate domain; a plurality of, e.g., up to 5, 10, 15, or 20, amino acid residues N terminal to its cognate domain; a plurality of, e.g., up to 5, 10, 15, or 20, amino acid residues C terminal to its cognate domain; a plurality of, e.g., up to 5, 10, 15, or 20, amino acid residues N terminal to its cognate domain and a plurality of e.g., up to 5, 10, 15, or 20, amino acid residues C terminal to its cognate domain.

In some embodiments, a deletion does not extend beyond: its cognate domain; the N terminal amino acid residue of its cognate domain; the C terminal amino acid residue of its cognate domain.

A REC-optimized Cas9 molecule or REC-optimized Cas9 polypeptide can include a linker disposed between the amino acid residues that flank the deletion. Suitable linkers for use between the amino acid resides that flank a REC deletion in a REC-optimized Cas9 molecule is described herein.

In some embodiments, a REC-optimized Cas9 molecule or REC-optimized Cas9 polypeptide comprises an amino acid sequence that, other than any REC deletion and associated linker, has at least 50, 55, 60, 65, 70, 75, 80, 85, 90, 95, 99, or 100% homology with the amino acid sequence of a naturally occurring Cas9, e.g., a S. aureus Cas9 molecule, a S. pyogenes Cas9 molecule, or a C. jejuni Cas9 molecule.

In some embodiments, a REC-optimized Cas9 molecule or REC-optimized Cas9 polypeptide comprises an amino acid sequence that, other than any REC deletion and associated linker, differs by no more than 1, 2, 3, 4, 5, 6, 7, 8, 9, 10, 15, 20, or 25, amino acid residues from the amino acid sequence of a naturally occurring Cas9, e.g., a S. aureus Cas9 molecule, a S. pyogenes Cas9 molecule, or a C. jejuni Cas9 molecule.

In some embodiments, a REC-optimized Cas9 molecule or REC-optimized Cas9 polypeptide comprises an amino acid sequence that, other than any REC deletion and associate linker, differs by no more than 1, 2, 3, 4, 5, 6, 7, 8, 9, 10, 15, 20, or 25% of the, amino acid residues from the amino acid sequence of a naturally occurring Cas9, e.g., a S. aureus Cas9 molecule, a S. pyogenes Cas9 molecule, or a C. jejuni Cas9 molecule.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. Methods of alignment of sequences for comparison are well known. Optimal alignment of sequences for comparison can be conducted, e.g., by the local homology algorithm of Smith and Waterman, (1970) Adv. Appl. Math. 2:482c, by the homology alignment algorithm of Needleman and Wunsch, (1970) J. Mol. Biol. 48:443, by the search for similarity method of Pearson and Lipman, (1988) Proc. Nat'l. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Brent et al., (2003) Current Protocols in Molecular Biology).

Two examples of algorithms that are suitable for determining percent sequence identity and sequence similarity are the BLAST and BLAST 2.0 algorithms, which are described in Altschul et al., (1977) Nuc. Acids Res. 25:3389-3402; and Altschul et al., (1990) J. Mol. Biol. 215:403-410, respectively. Software for performing BLAST analyses is publicly available through the National Center for Biotechnology Information.

The percent identity between two amino acid sequences can also be determined using the algorithm of E. Meyers and W. Miller, (1988) Comput. Appl. Biosci. 4:11-17) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (1970) J. Mol. Biol. 48:444-453) algorithm which has been incorporated into the GAP program in the GCG software package (available at www.gcg.com), using either a Blossom 62 matrix or a PAM250 matrix, and a gap weight of 16, 14, 12, 10, 8, 6, or 4 and a length weight of 1, 2, 3, 4, 5, or 6.

Sequence information for exemplary REC deletions are provided for 83 naturally-occurring Cas9 orthologs described in, e.g., International PCT Pub. Nos. WO2015/161276, WO2017/193107 and WO2017/093969.

(q) Nucleic Acids Encoding Cas9 Molecules

Nucleic acids encoding the Cas9 molecules or Cas9 polypeptides, e.g., an eaCas9 molecule or eaCas9 polypeptide, can be used in connection with any of the embodiments provided herein.

Exemplary nucleic acids encoding Cas9 molecules or Cas9 polypeptides are described in Cong et al., Science 2013, 399(6121):819-823; Wang et al., Cell 2013, 153(4):910-918; Mali et al., Science 2013, 399(6121):823-826; Jinek et al., Science 2012, 337(6096):816-821, and WO2015/161276, e.g., in FIG. 8 therein.

In some embodiments, a nucleic acid encoding a Cas9 molecule or Cas9 polypeptide can be a synthetic nucleic acid sequence. For example, the synthetic nucleic acid molecule can be chemically modified. In some embodiments, the Cas9 mRNA has one or more (e.g., all of the following properties: it is capped, polyadenylated, substituted with 5-methylcytidine and/or pseudouridine.

In addition, or alternatively, the synthetic nucleic acid sequence can be codon optimized, e.g., at least one non-common codon or less-common codon has been replaced by a common codon. For example, the synthetic nucleic acid can direct the synthesis of an optimized messenger mRNA, e.g., optimized for expression in a mammalian expression system, e.g., described herein.

In addition, or alternatively, a nucleic acid encoding a Cas9 molecule or Cas9 polypeptide may comprise a nuclear localization sequence (NLS). Nuclear localization sequences are known.

In some embodiments, the Cas9 molecule is encoded by a sequence that is or comprises any of SEQ ID NOS: 121, 123 or 125 or a sequence that exhibits at least 85%, 86%, 87%, 88%, 89%, 90%, 91%, 92%, 92%, 94%, 95%, 96%, 97%, 98%, 99% or more sequence identity to any of SEQ ID NOS: 121, 123 or 125. In some embodiments, the Cas9 molecule is or comprises any of SEQ ID NOs: 122, 124 or 125 or a sequence that exhibits at least 85%, 86%, 87%, 88%, 89%, 90%, 91%, 92%, 92%, 94%, 95%, 96%, 97%, 98%, 99% or more sequence identity to any of SEQ ID NOS: 122, 123 or 125. SEQ ID NO:121 is an exemplary codon optimized nucleic acid sequence encoding a Cas9 molecule of S. pyogenes. SEQ ID NO:122 is the corresponding amino acid sequence of a S. pyogenes Cas9 molecule. SEQ ID NO:123 is an exemplary codon optimized nucleic acid sequence encoding a Cas9 molecule of N. meningitidis. SEQ ID NO:124 is the corresponding amino acid sequence of a N. meningitidis Cas9 molecule. SEQ ID NO:125 is an exemplary codon optimized nucleic acid sequence encoding a Cas9 molecule of S. aureus Cas9. SEQ ID NO:126 is an amino acid sequence of a S. aureus Cas9 molecule.

If any of the foregoing Cas9 sequences are fused with a peptide or polypeptide at the C-terminus, it is understood that the stop codon will be removed.

(r) Other Cas Molecules and Cas Polypeptides

Various types of Cas molecules or Cas polypeptides can be used to practice the inventions disclosed herein. In some embodiments, Cas molecules of Type II Cas systems are used. In other embodiments, Cas molecules of other Cas systems are used. For example, Type I or Type III Cas molecules may be used. Exemplary Cas molecules (and Cas systems) are described, e.g., in Haft et al., PLoS Computational Biology 2005, 1(6): e60 and Makarova et al., Nature Review Microbiology 2011, 9:467-477, the contents of both references are incorporated herein by reference in their entirety. Exemplary Cas molecules (and Cas systems) are also shown in Table 3.

TABLE 3
Cas Systems

| | | | Structure of | Families (and | |
|---|---|---|---|---|---|
| | | | encoded | superfamily) of | |
| Gene | System type or | Name from | protein (PDB | encoded | |
| name^{‡} | subtype | Haft et al.^{§} | accessions)^{¶} | protein^{#}** | Representatives |
| cas1 | Type I | cas1 | 3GOD, 3LFX | COG1518 | SERP2463, |
| | Type II | | and 2YZS | | SPy1047 and ygbT |
| | Type III | | | | |
| cas2 | Type I | cas2 | 2IVY, 2I8E | COG1343 and | SERP2462, |
| | Type II | | and 3EXC | COG3512 | SPy1048, SPy1723 |
| | Type III | | | | (N-terminal |
| | | | | | domain) and ygbF |
| cas3′ | Type I^{‡‡} | cas3 | NA | COG1203 | APE1232 and ygcB |
| cas3″ | Subtype I-A | NA | NA | COG2254 | APE1231 and |
| | Subtype I-B | | | | BH0336 |
| cas4 | Subtype I-A | cas4 and | NA | COG1468 | APE1239 and |
| | Subtype I-B | csa1 | | | BH0340 |
| | Subtype I-C | | | | |
| | Subtype I-D | | | | |
| | Subtype II-B | | | | |
| cas5 | Subtype I-A | cas5a, | 3KG4 | COG1688 | APE1234, BH0337, |
| | Subtype I-B | cas5d, | | (RAMP) | devS and ygcI |
| | Subtype I-C | cas5e, | | | |
| | Subtype I-E | cas5h, | | | |
| | | cas5p, | | | |
| | | cas5t and | | | |
| | | cmx5 | | | |
| cas6 | Subtype I-A | cas6 and | 3I4H | COG1583 and | PF1131 and slr7014 |
| | Subtype I-B | cmx6 | | COG5551 | |
| | Subtype I-D | | | (RAMP) | |
| | Subtype III-A | | | | |
| | Subtype III-B | | | | |
| cas6e | Subtype I-E | cse3 | 1WJ9 | (RAMP) | ygcH |
| cas6f | Subtype I-F | csy4 | 2XLJ | (RAMP) | y1727 |
| cas7 | Subtype I-A | csa2, csd2, | NA | COG1857 and | devR and ygcJ |
| | Subtype I-B | cse4, csh2, | | COG3649 | |
| | Subtype I-C | csp1 and | | (RAMP) | |
| | Subtype I-E | cst2 | | | |
| cas8a1 | Subtype I-A^{‡‡} | cmx1, cst1, | NA | BH0338-like | LA3191^{§§} and |
| | | csx8, csx13 | | | PG2018^{§§} |
| | | and CXXC- | | | |
| | | CXXC | | | |
| cas8a2 | Subtype I-A^{‡‡} | csa4 and | NA | PH0918 | AF0070, AF1873, |
| | | csx9 | | | MJ0385, PF0637, |
| | | | | | PH0918 and |
| | | | | | SSO1401 |
| cas8b | Subtype I-B^{‡‡} | csh1 and | NA | BH0338-like | MTH1090 and |
| | | TM1802 | | | TM1802 |
| cas8c | Subtype I-C^{‡‡} | csd1 and | NA | BH0338-like | BH0338 |
| | | csp2 | | | |
| cas9 | Type II^{‡‡} | csn1 and | NA | COG3513 | FTN_0757 and |
| | | csx12 | | | SPy1046 |
| cas10 | Type III^{‡‡} | cmr2, csm1 | NA | COG1353 | MTH326, |
| | | and csx11 | | | Rv2823c^{§§} and |
| | | | | | TM1794^{§§} |
| cas10d | Subtype I-D^{‡‡} | csc3 | NA | COG1353 | slr7011 |
| csy1 | Subtype I-F^{‡‡} | csy1 | NA | y1724-like | y1724 |
| csy2 | Subtype I-F | csy2 | NA | (RAMP) | y1725 |
| csy3 | Subtype I-F | csy3 | NA | (RAMP) | y1726 |
| cse1 | Subtype I-E^{‡‡} | cse1 | NA | YgcL-like | ygcL |
| cse2 | Subtype I-E | cse2 | 2ZCA | YgcK-like | ygcK |
| csc1 | Subtype I-D | csc1 | NA | alr1563-like | alr1563 |
| | | | | (RAMP) | |
| csc2 | Subtype I-D | csc1 and | NA | COG1337 | slr7012 |
| | | csc2 | | (RAMP) | |
| csa5 | Subtype I-A | csa5 | NA | AF1870 | AF1870, MJ0380, |
| | | | | | PF0643 and |
| | | | | | SSO1398 |
| csn2 | Subtype II-A | csn2 | NA | SPy1049-like | SPy1049 |
| csm2 | Subtype III-A^{‡‡} | csm2 | NA | COG1421 | MTH1081 and |
| | | | | | SERP2460 |
| csm3 | Subtype III-A | csc2 and | NA | COG1337 | MTH1080 and |
| | | csm3 | | (RAMP) | SERP2459 |
| csm4 | Subtype III-A | csm4 | NA | COG1567 | MTH1079 and |
| | | | | (RAMP) | SERP2458 |
| csm5 | Subtype III-A | csm5 | NA | COG1332 | MTH1078 and |
| | | | | (RAMP) | SERP2457 |
| csm6 | Subtype III-A | APE2256 | 2WTE | COG1517 | APE2256 and |
| | | and csm6 | | | SSO1445 |
| cmr1 | Subtype III-B | cmr1 | NA | COG1367 | PF1130 |
| | | | | (RAMP) | |
| cmr3 | Subtype III-B | cmr3 | NA | COG1769 | PF1128 |
| | | | | (RAMP) | |
| cmr4 | Subtype III-B | cmr4 | NA | COG1336 | PF1126 |
| | | | | (RAMP) | |
| cmr5 | Subtype III-B^{‡‡} | cmr5 | 2ZOP and | COG3337 | MTH324 and |
| | | | 2OEB | | PF1125 |
| cmr6 | Subtype III-B | cmr6 | NA | COG1604 | PF1124 |
| | | | | (RAMP) | |
| csb1 | Subtype I-U | GSU0053 | NA | (RAMP) | Balac_1306 and |
| | | | | | GSU0053 |
| csb2 | Subtype I-U^{§§} | NA | NA | (RAMP) | Balac_1305 and |
| | | | | | GSU0054 |
| csb3 | Subtype I-U | NA | NA | (RAMP) | Balac_1303^{§§} |
| csx17 | Subtype I-U | NA | NA | NA | Btus_2683 |
| csx14 | Subtype I-U | NA | NA | NA | GSU0052 |
| csx10 | Subtype I-U | csx10 | NA | (RAMP) | Caur_2274 |
| csx16 | Subtype III-U | VVA1548 | NA | NA | VVA1548 |
| csaX | Subtype III-U | csaX | NA | NA | SSO1438 |
| csx3 | Subtype III-U | csx3 | NA | NA | AF1864 |
| csx1 | Subtype III-U | csa3, csx1, | 1XMX and | COG1517 and | MJ1666, NE0113, |
| | | csx2, | 2171 | COG4006 | PF1127 and |
| | | DXTHG, | | | TM1812 |
| | | NE0113 | | | |
| | | and | | | |
| | | TIGR02710 | | | |
| csx15 | Unknown | NA | NA | TTE2665 | TTE2665 |
| csf1 | Type U | csf1 | NA | NA | AFE_1038 |
| csf2 | Type U | csf2 | NA | (RAMP) | AFE_1039 |
| csf3 | Type U | csf3 | NA | (RAMP) | AFE_1040 |
| csf4 | Type U | csf4 | NA | NA | AFE_1037 |

(iii) Cpf1

In some embodiments, the guide RNA or gRNA promotes the specific association targeting of an RNA-guided nuclease such as a Cas9 or a Cpf1 to a target sequence such as a genomic or episomal sequence in a cell. In general, gRNAs can be unimolecular (comprising a single RNA molecule, and referred to alternatively as chimeric), or modular (comprising more than one, and typically two, separate RNA molecules, such as a crRNA and a tracrRNA, which are usually associated with one another, in some embodiments by duplexing). gRNAs and their component parts are described throughout the literature, in some embodiments in Briner et al. (Molecular Cell 56(2), 333-339, Oct. 23, 2014 (Briner), which is incorporated by reference), and in Cotta-Ramusino.

Guide RNAs, whether unimolecular or modular, generally include a targeting domain that is fully or partially complementary to a target, and are typically 10-30 nucleotides in length, and in certain embodiments are 16-24 nucleotides in length (in some embodiments, 16, 17, 18, 19, 20, 21, 22, 23 or 24 nucleotides in length). In some aspects, the targeting domains are at or near the 5′ terminus of the gRNA in the case of a Cas9 gRNA, and at or near the 3′ terminus in the case of a Cpf1 gRNA. While the foregoing description has focused on gRNAs for use with Cas9, it should be appreciated that other RNA-guided nucleases have been (or may in the future be) discovered or invented which utilize gRNAs that differ in some ways from those described to this point. In some embodiments, Cpf1 (“CRISPR from Prevotella and Franciscella 1”) is a recently discovered RNA-guided nuclease that does not require a tracrRNA to function. (Zetsche et al., 2015, Cell 163, 759-771 Oct. 22, 2015 (Zetsche I), incorporated by reference herein). A gRNA for use in a Cpf1 genome editing system generally includes a targeting domain and a complementarity domain (alternately referred to as a “handle”). It should also be noted that, in gRNAs for use with Cpf1, the targeting domain is usually present at or near the 3′ end, rather than the 5′ end as described above in connection with Cas9 gRNAs (the handle is at or near the 5′ end of a Cpf1 gRNA).

Although structural differences may exist between gRNAs from different prokaryotic species, or between Cpf1 and Cas9 gRNAs, the principles by which gRNAs operate are generally consistent. Because of this consistency of operation, gRNAs can be defined, in broad terms, by their targeting domain sequences, and skilled artisans will appreciate that a given targeting domain sequence can be incorporated in any suitable gRNA, including a unimolecular or chimeric gRNA, or a gRNA that includes one or more chemical modifications and/or sequential modifications (substitutions, additional nucleotides, truncations, etc.). Thus, in some aspects in this disclosure, gRNAs may be described solely in terms of their targeting domain sequences.

More generally, some aspects of the present disclosure relate to systems, methods and compositions that can be implemented using multiple RNA-guided nucleases. Unless otherwise specified, the term gRNA should be understood to encompass any suitable gRNA that can be used with any RNA-guided nuclease, and not only those gRNAs that are compatible with a particular species of Cas9 or Cpf1. By way of illustration, the term gRNA can, in certain embodiments, include a gRNA for use with any RNA-guided nuclease occurring in a Class 2 CRISPR system, such as a type II or type V or CRISPR system, or an RNA-guided nuclease derived or adapted therefrom.

Certain exemplary modifications discussed in this section can be included at any position within a gRNA sequence including, without limitation at or near the 5′ end (e.g., within 1-10, 1-5, or 1-2 nucleotides of the 5′ end) and/or at or near the 3′ end (e.g., within 1-10, 1-5, or 1-2 nucleotides of the 3′ end). In some cases, modifications are positioned within functional motifs, such as the repeat-anti-repeat duplex of a Cas9 gRNA, a stem loop structure of a Cas9 or Cpf1 gRNA, and/or a targeting domain of a gRNA.

RNA-guided nucleases include, but are not limited to, naturally-occurring Class 2 CRISPR nucleases such as Cas9, and Cpf1, as well as other nucleases derived or obtained therefrom. In functional terms, RNA-guided nucleases are defined as those nucleases that: (a) interact with (e.g complex with) a gRNA; and (b) together with the gRNA, associate with, and optionally cleave or modify, a target region of a DNA that includes (i) a sequence complementary to the targeting domain of the gRNA and, optionally, (ii) an additional sequence referred to as a “protospacer adjacent motif,” or “PAM,” which is described in greater detail below. As the following examples will illustrate, RNA-guided nucleases can be defined, in broad terms, by their PAM specificity and cleavage activity, even though variations may exist between individual RNA-guided nucleases that share the same PAM specificity or cleavage activity. Skilled artisans will appreciate that some aspects of the present disclosure relate to systems, methods and compositions that can be implemented using any suitable RNA-guided nuclease having a certain PAM specificity and/or cleavage activity. For this reason, unless otherwise specified, the term RNA-guided nuclease should be understood as a generic term, and not limited to any particular type (e.g. Cas9 vs. Cpf1), species (e.g. S. pyogenes vs. S. aureus) or variation (e.g full-length vs. truncated or split; naturally-occurring PAM specificity vs. engineered PAM specificity, etc.) of RNA-guided nuclease.

In addition to recognizing specific sequential orientations of PAMs and protospacers, RNA-guided nucleases in some embodiments can also recognize specific PAM sequences. S. aureus Cas9, in some embodiments, generally recognizes a PAM sequence of NNGRRT or NNGRRV, wherein the N residues are immediately 3′ of the region recognized by the gRNA targeting domain. S. pyogenes Cas9 generally recognizes NGG PAM sequences. And F. novicida Cpf1 generally recognizes a TTN PAM sequence.

The crystal structure of Acidaminococcus sp. Cpf1 in complex with crRNA and a double-stranded (ds) DNA target including a TTTN PAM sequence has been solved by Yamano et al. (Cell. 2016 May 5; 165(4): 949-962 (Yamano), incorporated by reference herein). Cpf1, like Cas9, has two lobes: a REC (recognition) lobe, and a NUC (nuclease) lobe. The REC lobe includes REC1 and REC2 domains, which lack similarity to any known protein structures. The NUC lobe, meanwhile, includes three RuvC domains (RuvC-I, -II and -III) and a BH domain. However, in contrast to Cas9, the Cpf1 REC lobe lacks an HNH domain, and includes other domains that also lack similarity to known protein structures: a structurally unique PI domain, three Wedge (WED) domains (WED-I, -II and -III), and a nuclease (Nuc) domain.

While Cas9 and Cpf1 share similarities in structure and function, it should be appreciated that certain Cpf1 activities are mediated by structural domains that are not analogous to any Cas9 domains. In some embodiments, cleavage of the complementary strand of the target DNA appears to be mediated by the Nuc domain, which differs sequentially and spatially from the HNH domain of Cas9. Additionally, the non-targeting portion of Cpf1 gRNA (the handle) adopts a pseudoknot structure, rather than a stem loop structure formed by the repeat:antirepeat duplex in Cas9 gRNAs.

Nucleic acids encoding RNA-guided nucleases, e.g., Cas9, Cpf1 or functional fragments thereof, are provided herein. Exemplary nucleic acids encoding RNA-guided nucleases have been described previously (see, e.g., Cong 2013; Wang 2013; Mali 2013; Jinek 2012).

b. Genome Editing Approaches

In general, it is to be understood that the alteration of any gene according to the methods described herein can be mediated by any mechanism and that any methods are not limited to a particular mechanism. Exemplary mechanisms that can be associated with the alteration of a gene include, but are not limited to, non-homologous end joining (e.g., classical or alternative), microhomology-mediated end joining (MMEJ), homology-directed repair (e.g., endogenous donor template mediated), synthesis dependent strand annealing (SDSA), single strand annealing, single strand invasion, single strand break repair (SSBR), mismatch repair (MMR), base excision repair (BER), Interstrand Crosslink (ICL) Translesion synthesis (TLS), or Error-free post-replication repair (PRR). Described herein are exemplary methods for targeted knockout of one or both alleles of the TGFBR2 locus.

1) NHEJ Approaches for Gene Targeting

As described herein, nuclease-induced non-homologous end-joining (NHEJ) can be used to target gene-specific knockouts. Nuclease-induced NHEJ can also be used to remove (e.g., delete) sequence insertions in a gene of interest.

While not wishing to be bound by theory, it is believed that, in some embodiments, the genomic alterations associated with the methods described herein rely on nuclease-induced NHEJ and the error-prone nature of the NHEJ repair pathway. NHEJ repairs a double-strand break in the DNA by joining together the two ends; however, generally, the original sequence is restored only if two compatible ends, exactly as they were formed by the double-strand break, are perfectly ligated. The DNA ends of the double-strand break are frequently the subject of enzymatic processing, resulting in the addition or removal of nucleotides, at one or both strands, prior to rejoining of the ends. This results in the presence of insertion and/or deletion (indel) mutations in the DNA sequence at the site of the NHEJ repair. Two-thirds of these mutations typically alter the reading frame and, therefore, produce a non-functional protein. Additionally, mutations that maintain the reading frame, but which insert or delete a significant amount of sequence, can destroy functionality of the protein. This is locus dependent as mutations in critical functional domains are likely less tolerable than mutations in non-critical regions of the protein. The indel mutations generated by NHEJ are unpredictable in nature; however, at a given break site certain indel sequences are favored and are over represented in the population, likely due to small regions of microhomology. The lengths of deletions can vary widely; most commonly in the 1-50 bp range, but they can easily reach greater than 100-200 bp. Insertions tend to be shorter and often include short duplications of the sequence immediately surrounding the break site. However, it is possible to obtain large insertions, and in these cases, the inserted sequence has often been traced to other regions of the genome or to plasmid DNA present in the cells.

Because NHEJ is a mutagenic process, it can also be used to delete small sequence motifs as long as the generation of a specific final sequence is not required. If a double-strand break is targeted near to a short target sequence, the deletion mutations caused by the NHEJ repair often span, and therefore remove, the unwanted nucleotides. For the deletion of larger DNA segments, introducing two double-strand breaks, one on each side of the sequence, can result in NHEJ between the ends with removal of the entire intervening sequence. In some embodiments, a pair of gRNAs can be used to introduce two double-strand breaks, resulting in a deletion of intervening sequences between the two breaks.

Both of these approaches can be used to delete specific DNA sequences; however, the error-prone nature of NHEJ may still produce indel mutations at the site of repair.

Both double strand cleaving eaCas9 molecules and single strand, or nickase, eaCas9 molecules can be used in the methods and compositions described herein to generate NHEJ-mediated indels. NHEJ-mediated indels targeted to the gene, e.g., a coding region, e.g., an early coding region of a gene, of interest can be used to knockout (i.e., eliminate expression of) a gene of interest. For example, early coding region of a gene of interest includes sequence immediately following a transcription start site, within a first exon of the coding sequence, or within 500 bp of the transcription start site (e.g., less than 500, 450, 400, 350, 300, 250, 200, 150, 100 or 50 bp).

In some embodiments, NHEJ-mediated indels are introduced into the TGFBR2 locus. Individual gRNAs or gRNA pairs targeting the gene are provided together with the Cas9 double-stranded nuclease or single-stranded nickase.

(1) Placement of Double Strand or Single Strand Breaks Relative to the Target Position

In some embodiments, in which a gRNA and Cas9 nuclease generate a double strand break for the purpose of inducing NHEJ-mediated indels, a gRNA, e.g., a unimolecular (or chimeric) or modular gRNA molecule, is configured to position one double-strand break in close proximity to a nucleotide of the target position. In some embodiments, the cleavage site is between 0-30 bp away from the target position (e.g., less than 30, 25, 20, 15, 10, 9, 8, 7, 6, 5, 4, 3, 2 or 1 bp from the target position).

In some embodiments, in which two gRNAs complexing with Cas9 nickases induce two single strand breaks for the purpose of inducing NHEJ-mediated indels, two gRNAs, e.g., independently, unimolecular (or chimeric) or modular gRNA, are configured to position two single-strand breaks to provide for NHEJ repair a nucleotide of the target position. In some embodiments, the gRNAs are configured to position cuts at the same position, or within a few nucleotides of one another, on different strands, essentially mimicking a double strand break. In some embodiments, the closer nick is between 0-30 bp away from the target position (e.g., less than 30, 25, 20, 15, 10, 9, 8, 7, 6, 5, 4, 3, 2 or 1 bp from the target position), and the two nicks are within 25-55 bp of each other (e.g., between 25 to 50, 25 to 45, 25 to 40, 25 to 35, 25 to 30, 50 to 55, 45 to 55, 40 to 55, 35 to 55, 30 to 55, 30 to 50, 35 to 50, 40 to 50, 45 to 50, 35 to 45, or 40 to 45 bp) and no more than 100 bp away from each other (e.g., no more than 90, 80, 70, 60, 50, 40, 30, 20 or 10 bp). In some embodiments, the gRNAs are configured to place a single strand break on either side of a nucleotide of the target position.

Both double strand cleaving eaCas9 molecules and single strand, or nickase, eaCas9 molecules can be used in the methods and compositions described herein to generate breaks both sides of a target position. Double strand or paired single strand breaks may be generated on both sides of a target position to remove the nucleic acid sequence between the two cuts (e.g., the region between the two breaks in deleted). In some embodiments, two gRNAs, e.g., independently, unimolecular (or chimeric) or modular gRNA, are configured to position a double-strand break on both sides of a target position. In an alternate embodiment, three gRNAs, e.g., independently, unimolecular (or chimeric) or modular gRNA, are configured to position a double strand break (i.e., one gRNA complexes with a cas9 nuclease) and two single strand breaks or paired single stranded breaks (i.e., two gRNAs complex with Cas9 nickases) on either side of the target position. In another embodiment, four gRNAs, e.g., independently, unimolecular (or chimeric) or modular gRNA, are configured to generate two pairs of single stranded breaks (i.e., two pairs of two gRNAs complex with Cas9 nickases) on either side of the target position. The double strand break(s) or the closer of the two single strand nicks in a pair will ideally be within 0-500 bp of the target position (e.g., no more than 450, 400, 350, 300, 250, 200, 150, 100, 50 or 25 bp from the target position). When nickases are used, the two nicks in a pair are within 25-55 bp of each other (e.g., between 25 to 50, 25 to 45, 25 to 40, 25 to 35, 25 to 30, 50 to 55, 45 to 55, 40 to 55, 35 to 55, 30 to 55, 30 to 50, 35 to 50, 40 to 50, 45 to 50, 35 to 45, or 40 to 45 bp) and no more than 100 bp away from each other (e.g., no more than 90, 80, 70, 60, 50, 40, 30, 20 or 10 bp).

2) Targeted Knockdown

Unlike CRISPR/Cas-mediated gene knockout, which permanently eliminates or reduces expression by mutating the gene at the DNA level, CRISPR/Cas knockdown allows for temporary reduction of gene expression through the use of artificial transcription factors. Mutating key residues in both DNA cleavage domains of the Cas9 protein (e.g., the D10A and H840A mutations) results in the generation of a catalytically inactive Cas9 (eiCas9 which is also known as dead Cas9 or dCas9). A catalytically inactive Cas9 complexes with a gRNA and localizes to the DNA sequence specified by that gRNA's targeting domain, however, it does not cleave the target DNA. Fusion of the dCas9 to an effector domain, e.g., a transcription repression domain, enables recruitment of the effector to any DNA site specified by the gRNA. While it has been shown that the eiCas9 itself can block transcription when recruited to early regions in the coding sequence, more robust repression can be achieved by fusing a transcriptional repression domain (for example KRAB, SID or ERD) to the Cas9 and recruiting it to the promoter region of a gene. It is likely that targeting DNase I hypersensitive regions of the promoter may yield more efficient gene repression or activation because these regions are more likely to be accessible to the Cas9 protein and are also more likely to harbor sites for endogenous transcription factors. Especially for gene repression, it is contemplated herein that blocking the binding site of an endogenous transcription factor would aid in downregulating gene expression. In another embodiment, an eiCas9 can be fused to a chromatin modifying protein. Altering chromatin status can result in decreased expression of the target gene.

In some embodiments, a gRNA molecule can be targeted to a known transcription response elements (e.g., promoters, enhancers, etc.), a known upstream activating sequences (UAS), and/or sequences of unknown or known function that are suspected of being able to control expression of the target DNA.

In some embodiments, CRISPR/Cas-mediated gene knockdown can be used to reduce expression one or more T-cell expressed genes. In some embodiments, in which a eiCas9 or an eiCas9 fusion protein described herein is used to knockdown the TGFBR2 locus, individual gRNAs or gRNA pairs targeting both or all genes are provided together with the eiCas9 or eiCas9 fusion protein.

3) Single-Strand Annealing

Single strand annealing (SSA) is another DNA repair process that repairs a double-strand break between two repeat sequences present in a target nucleic acid. Repeat sequences utilized by the SSA pathway are generally greater than 30 nucleotides in length. Resection at the break ends occurs to reveal repeat sequences on both strands of the target nucleic acid. After resection, single strand overhangs containing the repeat sequences are coated with RPA protein to prevent the repeats sequences from inappropriate annealing, e.g., to themselves. RAD52 binds to and each of the repeat sequences on the overhangs and aligns the sequences to enable the annealing of the complementary repeat sequences. After annealing, the single-strand flaps of the overhangs are cleaved. New DNA synthesis fills in any gaps, and ligation restores the DNA duplex. As a result of the processing, the DNA sequence between the two repeats is deleted. The length of the deletion can depend on many factors including the location of the two repeats utilized, and the pathway or processivity of the resection.

In contrast to HDR pathways, SSA does not require a template nucleic acid to alter or correct a target nucleic acid sequence. Instead, the complementary repeat sequence is utilized.

4) Other DNA Repair Pathways

A) SSBR (Single Strand Break Repair)

Single-stranded breaks (SSB) in the genome are repaired by the SSBR pathway, which is a distinct mechanism from the DSB repair mechanisms discussed above. The SSBR pathway has four major stages: SSB detection, DNA end processing, DNA gap filling, and DNA ligation. A more detailed explanation is given in Caldecott, Nature Reviews Genetics 9, 619-631 (August 2008), and a summary is given here.

In the first stage, when a SSB forms, PARP1 and/or PARP2 recognize the break and recruit repair machinery. The binding and activity of PARP1 at DNA breaks is transient and it seems to accelerate SSBr by promoting the focal accumulation or stability of SSBr protein complexes at the lesion. Arguably the most important of these SSBr proteins is XRCC1, which functions as a molecular scaffold that interacts with, stabilizes, and stimulates multiple enzymatic components of the SSBr process including the protein responsible for cleaning the DNA 3′ and 5′ ends. In some embodiments, XRCC1 interacts with several proteins (DNA polymerase beta, PNK, and three nucleases, APE1, APTX, and APLF) that promote end processing. APE1 has endonuclease activity. APLF exhibits endonuclease and 3′ to 5′ exonuclease activities. APTX has endonuclease and 3′ to 5′ exonuclease activity.

This end processing is an important stage of SSBR since the 3′- and/or 5′-termini of most, if not all, SSBs are ‘damaged’. End processing generally involves restoring a damaged 3′-end to a hydroxylated state and and/or a damaged 5′ end to a phosphate moiety, so that the ends become ligation-competent. Enzymes that can process damaged 3′ termini include PNKP, APE1, and TDP1. Enzymes that can process damaged 5′ termini include PNKP, DNA polymerase beta, and APTX. LIG3 (DNA ligase III) can also participate in end processing. Once the ends are cleaned, gap filling can occur.

At the DNA gap filling stage, the proteins typically present are PARP1, DNA polymerase beta, XRCC1, FEN1 (flap endonuclease 1), DNA polymerase delta/epsilon, PCNA, and LIG1. There are two ways of gap filling, the short patch repair and the long patch repair. Short patch repair involves the insertion of a single nucleotide that is missing. At some SSBs, “gap filling” might continue displacing two or more nucleotides (displacement of up to 12 bases have been reported). FEN1 is an endonuclease that removes the displaced 5′-residues. Multiple DNA polymerases, including Pol 3, are involved in the repair of SSBs, with the choice of DNA polymerase influenced by the source and type of SSB.

In the fourth stage, a DNA ligase such as LIG1 (Ligase I) or LIG3 (Ligase III) catalyzes joining of the ends. Short patch repair uses Ligase III and long patch repair uses Ligase I.

Sometimes, SSBR is replication-coupled. This pathway can involve one or more of CtIP, MRN, ERCC1, and FEN1. Additional factors that may promote SSBR include: aPARP, PARP1, PARP2, PARG, XRCC1, DNA polymerase b, DNA polymerase d, DNA polymerase e, PCNA, LIG1, PNK, PNKP, APE1, APTX, APLF, TDP1, LIG3, FEN1, CtIP, MRN, and ERCC1.

B) MMR (Mismatch Repair)

Cells contain three excision repair pathways: MMR, BER, and NER. The excision repair pathways have a common feature in that they typically recognize a lesion on one strand of the DNA, then exo/endonucleaseases remove the lesion and leave a 1-30 nucleotide gap that is sub-sequentially filled in by DNA polymerase and finally sealed with ligase. A more complete picture is given in Li, Cell Research (2008) 18:85-98, and a summary is provided here.

Mismatch repair (MMR) operates on mispaired DNA bases. The MSH2/6 or MSH2/3 complexes both have ATPases activity that plays an important role in mismatch recognition and the initiation of repair. MSH2/6 preferentially recognizes base-base mismatches and identifies mispairs of 1 or 2 nucleotides, while MSH2/3 preferentially recognizes larger ID mispairs.

hMLH1 heterodimerizes with hPMS2 to form hMutLa which possesses an ATPase activity and is important for multiple steps of MMR. It possesses a PCNA/replication factor C (RFC)-dependent endonuclease activity which plays an important role in 3′ nick-directed MMR involving EXO1. (EXO1 is a participant in both HR and MMR.) It regulates termination of mismatch-provoked excision. Ligase I is the relevant ligase for this pathway. Additional factors that may promote MMR include: EXO1, MSH2, MSH3, MSH6, MLH1, PMS2, MLH3, DNA Pol d, RPA, HMGB1, RFC, and DNA ligase I.

C) Base Excision Repair (BER)

The base excision repair (BER) pathway is active throughout the cell cycle; it is responsible primarily for removing small, non-helix-distorting base lesions from the genome. In contrast, the related Nucleotide Excision Repair pathway (discussed in the next section) repairs bulky helix-distorting lesions. A more detailed explanation is given in Caldecott, Nature Reviews Genetics 9, 619-631 (August 2008), and a summary is given here.

Upon DNA base damage, base excision repair (BER) is initiated and the process can be simplified into five major steps: (a) removal of the damaged DNA base; (b) incision of the subsequent a basic site; (c) clean-up of the DNA ends; (d) insertion of the correct nucleotide into the repair gap; and (e) ligation of the remaining nick in the DNA backbone. These last steps are similar to the SSBR.

In the first step, a damage-specific DNA glycosylase excises the damaged base through cleavage of the N-glycosidic bond linking the base to the sugar phosphate backbone. Then AP endonuclease-1 (APE1) or bifunctional DNA glycosylases with an associated lyase activity incised the phosphodiester backbone to create a DNA single strand break (SSB). The third step of BER involves cleaning-up of the DNA ends. The fourth step in BER is conducted by Pol R that adds a new complementary nucleotide into the repair gap and in the final step XRCC1/Ligase III seals the remaining nick in the DNA backbone. This completes the short-patch BER pathway in which the majority (˜80%) of damaged DNA bases are repaired. However, if the 5′-ends in step 3 are resistant to end processing activity, following one nucleotide insertion by Pol β there is then a polymerase switch to the replicative DNA polymerases, Pol δ/ε, which then add ˜2-8 more nucleotides into the DNA repair gap. This creates a 5′-flap structure, which is recognized and excised by flap endonuclease-1 (FEN-1) in association with the processivity factor proliferating cell nuclear antigen (PCNA). DNA ligase I then seals the remaining nick in the DNA backbone and completes long-patch BER. Additional factors that may promote the BER pathway include: DNA glycosylase, APE1, Polb, Pold, Pole, XRCC1, Ligase III, FEN-1, PCNA, RECQL4, WRN, MYH, PNKP, and APTX.

D) Nucleotide Excision Repair (NER)

Nucleotide excision repair (NER) is an important excision mechanism that removes bulky helix-distorting lesions from DNA. Additional details about NER are given in Marteijn et al., Nature Reviews Molecular Cell Biology 15, 465-481 (2014), and a summary is given here. NER a broad pathway encompassing two smaller pathways: global genomic NER (GG-NER) and transcription coupled repair NER (TC-NER). GG-NER and TC-NER use different factors for recognizing DNA damage. However, they utilize the same machinery for lesion incision, repair, and ligation.

Once damage is recognized, the cell removes a short single-stranded DNA segment that contains the lesion. Endonucleases XPF/ERCC1 and XPG (encoded by ERCC5) remove the lesion by cutting the damaged strand on either side of the lesion, resulting in a single-strand gap of 22-30 nucleotides. Next, the cell performs DNA gap filling synthesis and ligation. Involved in this process are: PCNA, RFC, DNA Pol δ, DNA Pol ε or DNA Pol κ, and DNA ligase I or XRCC1/Ligase III. Replicating cells tend to use DNA pol E and DNA ligase I, while non-replicating cells tend to use DNA Pol δ, DNA Pol κ, and the XRCC1/Ligase III complex to perform the ligation step.

NER can involve the following factors: XPA-G, POLH, XPF, ERCC1, XPA-G, and LIG1. Transcription-coupled NER (TC-NER) can involve the following factors: CSA, CSB, XPB, XPD, XPG, ERCC1, and TTDA. Additional factors that may promote the NER repair pathway include XPA-G, POLH, XPF, ERCC1, XPA-G, LIG1, CSA, CSB, XPA, XPB, XPC, XPD, XPF, XPG, TTDA, UVSSA, USP7, CETN2, RAD23B, UV-DDB, CAK subcomplex, RPA, and PCNA.

E) Interstrand Crosslink (ICL)

A dedicated pathway called the ICL repair pathway repairs interstrand crosslinks. Interstrand crosslinks, or covalent crosslinks between bases in different DNA strand, can occur during replication or transcription. ICL repair involves the coordination of multiple repair processes, in particular, nucleolytic activity, translesion synthesis (TLS), and HDR. Nucleases are recruited to excise the ICL on either side of the crosslinked bases, while TLS and HDR are coordinated to repair the cut strands. ICL repair can involve the following factors: endonucleases, e.g., XPF and RAD51C, endonucleases such as RAD51, translesion polymerases, e.g., DNA polymerase zeta and Rev1), and the Fanconi anemia (FA) proteins, e.g., FancJ.

F) Other pathways

Several other DNA repair pathways exist in mammals. Translesion synthesis (TLS) is a pathway for repairing a single stranded break left after a defective replication event and involves translesion polymerases, e.g., DNA pol(and Rev1. Error-free post replication repair (PRR) is another pathway for repairing a single stranded break left after a defective replication event.

5) Examples of gRNAs in Genome Editing Methods

Any of the gRNA molecules as described herein can be used with any Cas9 molecules that generate a double strand break or a single strand break to alter the sequence of a target nucleic acid, e.g., a target position or target genetic signature. In some examples, the target nucleic acid is at or near the TGFBR2 locus, such as any as described. In some embodiments, a ribonucleic acid molecule, such as a gRNA molecule, and a protein, such as a Cas9 protein or variants thereof, are introduced to any of the engineered cells provided herein. gRNA molecules useful in these methods are described below.

In some embodiments, the gRNA, e.g., a chimeric gRNA, is configured such that it comprises one or more of the following properties;

a) it can position, e.g., when targeting a Cas9 molecule that makes double strand breaks, a double strand break (i) within 50, 100, 150, 200, 250, 300, 350, 400, 450, or 500 nucleotides of a target position, or (ii) sufficiently close that the target position is within the region of end resection;

b) it has a targeting domain of at least 16 nucleotides, e.g., a targeting domain of (i) 16, (ii), 17, (iii) 18, (iv) 19, (v) 20, (vi) 21, (vii) 22, (viii) 23, (ix) 24, (x) 25, or (xi) 26 nucleotides; and

c) (i) the proximal and tail domain, when taken together, comprise at least 15, 18, 20, 25, 30, 31, 35, 40, 45, 49, 50, or 53 nucleotides, e.g., at least 15, 18, 20, 25, 30, 31, 35, 40, 45, 49, 50, or 53 nucleotides from a naturally occurring S. pyogenes, S. thermophilus, S. aureus, or N. meningitidis tail and proximal domain, or a sequence that differs by no more than 1, 2, 3, 4, 5, 6, 7, 8, 9 or 10 nucleotides therefrom;

(ii) there are at least 15, 18, 20, 25, 30, 31, 35, 40, 45, 49, 50, or 53 nucleotides 3′ to the last nucleotide of the second complementarity domain, e.g., at least 15, 18, 20, 25, 30, 31, 35, 40, 45, 49, 50, or 53 nucleotides from the corresponding sequence of a naturally occurring S. pyogenes, S. thermophilus, S. aureus, or N. meningitidis gRNA, or a sequence that differs by no more than 1, 2, 3, 4, 5; 6, 7, 8, 9 or 10 nucleotides therefrom;

(iii) there are at least 16, 19, 21, 26, 31, 32, 36, 41, 46, 50, 51, or 54 nucleotides 3′ to the last nucleotide of the second complementarity domain that is complementary to its corresponding nucleotide of the first complementarity domain, e.g., at least 16, 19, 21, 26, 31, 32, 36, 41, 46, 50, 51, or 54 nucleotides from the corresponding sequence of a naturally occurring S. pyogenes, S. thermophilus, S. aureus, or N. meningitidis gRNA, or a sequence that differs by no more than 1, 2, 3, 4, 5; 6, 7, 8, 9 or 10 nucleotides therefrom;

(iv) the tail domain is at least 10, 15, 20, 25, 30, 35 or 40 nucleotides in length, e.g., it comprises at least 10, 15, 20, 25, 30, 35 or 40 nucleotides from a naturally occurring S. pyogenes, S. thermophilus, S. aureus, or N. meningitidis tail domain, or a sequence that differs by no more than 1, 2, 3, 4, 5; 6, 7, 8, 9 or 10 nucleotides therefrom; or

(v) the tail domain comprises 15, 20, 25, 30, 35, 40 nucleotides or all of the corresponding portions of a naturally occurring tail domain, e.g., a naturally occurring S. pyogenes, S. thermophilus, S. aureus, or N. meningitidis tail domain.

In some embodiments, the gRNA is configured such that it comprises properties: a and b(i). In some embodiments, the gRNA is configured such that it comprises properties: a and b(ii). In some embodiments, the gRNA is configured such that it comprises properties: a and b(iii). In some embodiments, the gRNA is configured such that it comprises properties: a and b(iv). In some embodiments, the gRNA is configured such that it comprises properties: a and b(v). In some embodiments, the gRNA is configured such that it comprises properties: a and b(vi). In some embodiments, the gRNA is configured such that it comprises properties: a and b(vii). In some embodiments, the gRNA is configured such that it comprises properties: a and b(viii). In some embodiments, the gRNA is configured such that it comprises properties: a and b(ix). In some embodiments, the gRNA is configured such that it comprises properties: a and b(x). In some embodiments, the gRNA is configured such that it comprises properties: a and b(xi). In some embodiments, the gRNA is configured such that it comprises properties: a and c. In some embodiments, the gRNA is configured such that in comprises properties: a, b, and c. In some embodiments, the gRNA is configured such that in comprises properties: a(i), b(i), and c(i). In some embodiments, the gRNA is configured such that in comprises properties: a(i), b(i), and c(ii). In some embodiments, the gRNA is configured such that in comprises properties: a(i), b(ii), and c(i). In some embodiments, the gRNA is configured such that in comprises properties: a(i), b(ii), and c(ii). In some embodiments, the gRNA is configured such that in comprises properties: a(i), b(iii), and c(i). In some embodiments, the gRNA is configured such that in comprises properties: a(i), b(iii), and c(ii). In some embodiments, the gRNA is configured such that in comprises properties: a(i), b(iv), and c(i). In some embodiments, the gRNA is configured such that in comprises properties: a(i), b(iv), and c(ii). In some embodiments, the gRNA is configured such that in comprises properties: a(i), b(v), and c(i). In some embodiments, the gRNA is configured such that in comprises properties: a(i), b(v), and c(ii). In some embodiments, the gRNA is configured such that in comprises properties: a(i), b(vi), and c(i). In some embodiments, the gRNA is configured such that in comprises properties: a(i), b(vi), and c(ii). In some embodiments, the gRNA is configured such that in comprises properties: a(i), b(vii), and c(i). In some embodiments, the gRNA is configured such that in comprises properties: a(i), b(vii), and c(ii). In some embodiments, the gRNA is configured such that in comprises properties: a(i), b(viii), and c(i). In some embodiments, the gRNA is configured such that in comprises properties: a(i), b(viii), and c(ii). In some embodiments, the gRNA is configured such that in comprises properties: a(i), b(ix), and c(i). In some embodiments, the gRNA is configured such that in comprises properties: a(i), b(ix), and c(ii). In some embodiments, the gRNA is configured such that in comprises properties: a(i), b(x), and c(i). In some embodiments, the gRNA is configured such that in comprises properties: a(i), b(x), and c(ii). In some embodiments, the gRNA is configured such that in comprises properties: a(i), b(xi), and c(i). In some embodiments, the gRNA is configured such that in comprises properties: a(i), b(xi), and c(ii).

In some embodiments, the gRNA, e.g., a chimeric gRNA, is configured such that it comprises one or more of the following properties;

a) one or both of the gRNAs can position, e.g., when targeting a Cas9 molecule that makes single strand breaks, a single strand break within (i) 50, 100, 150, 200, 250, 300, 350, 400, 450, or 500 nucleotides of a target position, or (ii) sufficiently close that the target position is within the region of end resection;

b) one or both have a targeting domain of at least 16 nucleotides, e.g., a targeting domain of (i) 16, (ii), 17, (iii) 18, (iv) 19, (v) 20, (vi) 21, (vii) 22, (viii) 23, (ix) 24, (x) 25, or (xi) 26 nucleotides; and

c) (i) the proximal and tail domain, when taken together, comprise at least 15, 18, 20, 25, 30, 31, 35, 40, 45, 49, 50, or 53 nucleotides, e.g., at least 15, 18, 20, 25, 30, 31, 35, 40, 45, 49, 50, or 53 nucleotides from a naturally occurring S. pyogenes, S. thermophilus, S. aureus, or N. meningitidis tail and proximal domain, or a sequence that differs by no more than 1, 2, 3, 4, 5; 6, 7, 8, 9 or 10 nucleotides therefrom;

(ii) there are at least 15, 18, 20, 25, 30, 31, 35, 40, 45, 49, 50, or 53 nucleotides 3′ to the last nucleotide of the second complementarity domain, e.g., at least 15, 18, 20, 25, 30, 31, 35, 40, 45, 49, 50, or 53 nucleotides from the corresponding sequence of a naturally occurring S. pyogenes, S. thermophilus, S. aureus, or N. meningitidis gRNA, or a sequence that differs by no more than 1, 2, 3, 4, 5; 6, 7, 8, 9 or 10 nucleotides therefrom;

(iii) there are at least 16, 19, 21, 26, 31, 32, 36, 41, 46, 50, 51, or 54 nucleotides 3′ to the last nucleotide of the second complementarity domain that is complementary to its corresponding nucleotide of the first complementarity domain, e.g., at least 16, 19, 21, 26, 31, 32, 36, 41, 46, 50, 51, or 54 nucleotides from the corresponding sequence of a naturally occurring S. pyogenes, S. thermophilus, S. aureus, or N. meningitidis gRNA, or a sequence that differs by no more than 1, 2, 3, 4, 5; 6, 7, 8, 9 or 10 nucleotides therefrom;

(iv) the tail domain is at least 10, 15, 20, 25, 30, 35 or 40 nucleotides in length, e.g., it comprises at least 10, 15, 20, 25, 30, 35 or 40 nucleotides from a naturally occurring S. pyogenes, S. thermophilus, S. aureus, or N. meningitidis tail domain, or a sequence that differs by no more than 1, 2, 3, 4, 5; 6, 7, 8, 9 or 10 nucleotides therefrom; or

(v) the tail domain comprises 15, 20, 25, 30, 35, 40 nucleotides or all of the corresponding portions of a naturally occurring tail domain, e.g., a naturally occurring S. pyogenes, S. thermophilus, S. aureus, or N. meningitidis tail domain.

In some embodiments, the gRNA is configured such that it comprises properties: a and b(i). In some embodiments, the gRNA is configured such that it comprises properties: a and b(ii). In some embodiments, the gRNA is configured such that it comprises properties: a and b(iii). In some embodiments, the gRNA is configured such that it comprises properties: a and b(iv). In some embodiments, the gRNA is configured such that it comprises properties: a and b(v). In some embodiments, the gRNA is configured such that it comprises properties: a and b(vi). In some embodiments, the gRNA is configured such that it comprises properties: a and b(vii). In some embodiments, the gRNA is configured such that it comprises properties: a and b(viii). In some embodiments, the gRNA is configured such that it comprises properties: a and b(ix). In some embodiments, the gRNA is configured such that it comprises properties: a and b(x). In some embodiments, the gRNA is configured such that it comprises properties: a and b(xi). In some embodiments, the gRNA is configured such that it comprises properties: a and c. In some embodiments, the gRNA is configured such that in comprises properties: a, b, and c. In some embodiments, the gRNA is configured such that in comprises properties: a(i), b(i), and c(i). In some embodiments, the gRNA is configured such that in comprises properties: a(i), b(i), and c(ii). In some embodiments, the gRNA is configured such that in comprises properties: a(i), b(ii), and c(i). In some embodiments, the gRNA is configured such that in comprises properties: a(i), b(ii), and c(ii). In some embodiments, the gRNA is configured such that in comprises properties: a(i), b(iii), and c(i). In some embodiments, the gRNA is configured such that in comprises properties: a(i), b(iii), and c(ii). In some embodiments, the gRNA is configured such that in comprises properties: a(i), b(iv), and c(i). In some embodiments, the gRNA is configured such that in comprises properties: a(i), b(iv), and c(ii). In some embodiments, the gRNA is configured such that in comprises properties: a(i), b(v), and c(i). In some embodiments, the gRNA is configured such that in comprises properties: a(i), b(v), and c(ii). In some embodiments, the gRNA is configured such that in comprises properties: a(i), b(vi), and c(i). In some embodiments, the gRNA is configured such that in comprises properties: a(i), b(vi), and c(ii). In some embodiments, the gRNA is configured such that in comprises properties: a(i), b(vii), and c(i). In some embodiments, the gRNA is configured such that in comprises properties: a(i), b(vii), and c(ii). In some embodiments, the gRNA is configured such that in comprises properties: a(i), b(viii), and c(i). In some embodiments, the gRNA is configured such that in comprises properties: a(i), b(viii), and c(ii). In some embodiments, the gRNA is configured such that in comprises properties: a(i), b(ix), and c(i). In some embodiments, the gRNA is configured such that in comprises properties: a(i), b(ix), and c(ii). In some embodiments, the gRNA is configured such that in comprises properties: a(i), b(x), and c(i). In some embodiments, the gRNA is configured such that in comprises properties: a(i), b(x), and c(ii). In some embodiments, the gRNA is configured such that in comprises properties: a(i), b(xi), and c(i). In some embodiments, the gRNA is configured such that in comprises properties: a(i), b(xi), and c(ii).

In some embodiments, the gRNA is used with a Cas9 nickase molecule having HNH activity, e.g., a Cas9 molecule having the RuvC activity inactivated, e.g., a Cas9 molecule having a mutation at D10, e.g., the D10A mutation.

In some embodiments, the gRNA is used with a Cas9 nickase molecule having RuvC activity, e.g., a Cas9 molecule having the HNH activity inactivated, e.g., a Cas9 molecule having a mutation at H840, e.g., a H840A.

In some embodiments, a pair of gRNAs, e.g., a pair of chimeric gRNAs, comprising a first and a second gRNA, is configured such that they comprises one or more of the following properties;

a) one or both of the gRNAs can position, e.g., when targeting a Cas9 molecule that makes single strand breaks, a single strand break within (i) 50, 100, 150, 200, 250, 300, 350, 400, 450, or 500 nucleotides of a target position, or (ii) sufficiently close that the target position is within the region of end resection;

b) one or both have a targeting domain of at least 16 nucleotides, e.g., a targeting domain of (i) 16, (ii), 17, (iii) 18, (iv) 19, (v) 20, (vi) 21, (vii) 22, (viii) 23, (ix) 24, (x) 25, or (xi) 26 nucleotides; c) for one or both:

(i) the proximal and tail domain, when taken together, comprise at least 15, 18, 20, 25, 30, 31, 35, 40, 45, 49, 50, or 53 nucleotides, e.g., at least 15, 18, 20, 25, 30, 31, 35, 40, 45, 49, 50, or 53 nucleotides from a naturally occurring S. pyogenes, S. thermophilus, S. aureus, or N. meningitidis tail and proximal domain, or a sequence that differs by no more than 1, 2, 3, 4, 5; 6, 7, 8, 9 or 10 nucleotides therefrom;

(ii) there are at least 15, 18, 20, 25, 30, 31, 35, 40, 45, 49, 50, or 53 nucleotides 3′ to the last nucleotide of the second complementarity domain, e.g., at least 15, 18, 20, 25, 30, 31, 35, 40, 45, 49, 50, or 53 nucleotides from the corresponding sequence of a naturally occurring S. pyogenes, S. thermophilus, S. aureus, or N. meningitidis gRNA, or a sequence that differs by no more than 1, 2, 3, 4, 5; 6, 7, 8, 9 or 10 nucleotides therefrom;

(iii) there are at least 16, 19, 21, 26, 31, 32, 36, 41, 46, 50, 51, or 54 nucleotides 3′ to the last nucleotide of the second complementarity domain that is complementary to its corresponding nucleotide of the first complementarity domain, e.g., at least 16, 19, 21, 26, 31, 32, 36, 41, 46, 50, 51, or 54 nucleotides from the corresponding sequence of a naturally occurring S. pyogenes, S. thermophilus, S. aureus, or N. meningitidis gRNA, or a sequence that differs by no more than 1, 2, 3, 4, 5; 6, 7, 8, 9 or 10 nucleotides therefrom;

(iv) the tail domain is at least 10, 15, 20, 25, 30, 35 or 40 nucleotides in length, e.g., it comprises at least 10, 15, 20, 25, 30, 35 or 40 nucleotides from a naturally occurring S. pyogenes, S. thermophilus, S. aureus, or N. meningitidis tail domain; or, or a sequence that differs by no more than 1, 2, 3, 4, 5; 6, 7, 8, 9 or 10 nucleotides therefrom; or

(v) the tail domain comprises 15, 20, 25, 30, 35, 40 nucleotides or all of the corresponding portions of a naturally occurring tail domain, e.g., a naturally occurring S. pyogenes, S. thermophilus, S. aureus, or N. meningitidis tail domain;

d) the gRNAs are configured such that, when hybridized to target nucleic acid, they are separated by 0-50, 0-100, 0-200, at least 10, at least 20, at least 30 or at least 50 nucleotides;

e) the breaks made by the first gRNA and second gRNA are on different strands; and

f) the PAMs are facing outwards.

In some embodiments, one or both of the gRNAs is configured such that it comprises properties: a and b(i). In some embodiments, one or both of the gRNAs is configured such that it comprises properties: a and b(ii). In some embodiments, one or both of the gRNAs is configured such that it comprises properties: a and b(iii). In some embodiments, one or both of the gRNAs is configured such that it comprises properties: a and b(iv). In some embodiments, one or both of the gRNAs is configured such that it comprises properties: a and b(v). In some embodiments, one or both of the gRNAs is configured such that it comprises properties: a and b(vi). In some embodiments, one or both of the gRNAs is configured such that it comprises properties: a and b(vii). In some embodiments, one or both of the gRNAs is configured such that it comprises properties: a and b(viii). In some embodiments, one or both of the gRNAs is configured such that it comprises properties: a and b(ix). In some embodiments, one or both of the gRNAs is configured such that it comprises properties: a and b(x). In some embodiments, one or both of the gRNAs is configured such that it comprises properties: a and b(xi). In some embodiments, one or both of the gRNAs configured such that it comprises properties: a and c. In some embodiments, one or both of the gRNAs is configured such that it comprises properties: a, b, and c. In some embodiments, one or both of the gRNAs is configured such that it comprises properties: a(i), b(i), and c(i). In some embodiments, one or both of the gRNAs is configured such that it comprises properties: a(i), b(i), and c(ii). In some embodiments, one or both of the gRNAs is configured such that it comprises properties: a(i), b(i), c, and d. In some embodiments, one or both of the gRNAs is configured such that it comprises properties: a(i), b(i), c, and e. In some embodiments, one or both of the gRNAs is configured such that it comprises properties: a(i), b(i), c, d, and e. In some embodiments, one or both of the gRNAs is configured such that it comprises properties: a(i), b(ii), and c(i). In some embodiments, one or both of the gRNAs is configured such that it comprises properties: a(i), b(ii), and c(ii). In some embodiments, one or both of the gRNAs is configured such that it comprises properties: a(i), b(ii), c, and d. In some embodiments, one or both of the gRNAs is configured such that it comprises properties: a(i), b(ii), c, and e. In some embodiments, one or both of the gRNAs is configured such that it comprises properties: a(i), b(ii), c, d, and e. In some embodiments, one or both of the gRNAs is configured such that it comprises properties: a(i), b(iii), and c(i). In some embodiments, one or both of the gRNAs is configured such that it comprises properties: a(i), b(iii), and c(ii). In some embodiments, one or both of the gRNAs is configured such that it comprises properties: a(i), b(iii), c, and d. In some embodiments, one or both of the gRNAs is configured such that it comprises properties: a(i), b(iii), c, and e. In some embodiments, one or both of the gRNAs is configured such that it comprises properties: a(i), b(iii), c, d, and e. In some embodiments, one or both of the gRNAs is configured such that it comprises properties: a(i), b(iv), and c(i). In some embodiments, one or both of the gRNAs is configured such that it comprises properties: a(i), b(iv), and c(ii). In some embodiments, one or both of the gRNAs is configured such that it comprises properties: a(i), b(iv), c, and d. In some embodiments, one or both of the gRNAs is configured such that it comprises properties: a(i), b(iv), c, and e. In some embodiments, one or both of the gRNAs is configured such that it comprises properties: a(i), b(iv), c, d, and e. In some embodiments, one or both of the gRNAs is configured such that it comprises properties: a(i), b(v), and c(i). In some embodiments, one or both of the gRNAs is configured such that it comprises properties: a(i), b(v), and c(ii). In some embodiments, one or both of the gRNAs is configured such that it comprises properties: a(i), b(v), c, and d. In some embodiments, one or both of the gRNAs is configured such that it comprises properties: a(i), b(v), c, and e. In some embodiments, one or both of the gRNAs is configured such that it comprises properties: a(i), b(v), c, d, and e. In some embodiments, one or both of the gRNAs is configured such that it comprises properties: a(i), b(vi), and c(i). In some embodiments, one or both of the gRNAs is configured such that it comprises properties: a(i), b(vi), and c(ii). In some embodiments, one or both of the gRNAs is configured such that it comprises properties: a(i), b(vi), c, and d. In some embodiments, one or both of the gRNAs is configured such that it comprises properties: a(i), b(vi), c, and e. In some embodiments, one or both of the gRNAs is configured such that it comprises properties: a(i), b(vi), c, d, and e. In some embodiments, one or both of the gRNAs is configured such that it comprises properties: a(i), b(vii), and c(i). In some embodiments, one or both of the gRNAs is configured such that it comprises properties: a(i), b(vii), and c(ii). In some embodiments, one or both of the gRNAs is configured such that it comprises properties: a(i), b(vii), c, and d. In some embodiments, one or both of the gRNAs is configured such that it comprises properties: a(i), b(vii), c, and e. In some embodiments, one or both of the gRNAs is configured such that it comprises properties: a(i), b(vii), c, d, and e. In some embodiments, one or both of the gRNAs is configured such that it comprises properties: a(i), b(viii), and c(i). In some embodiments, one or both of the gRNAs is configured such that it comprises properties: a(i), b(viii), and c(ii). In some embodiments, one or both of the gRNAs is configured such that it comprises properties: a(i), b(viii), c, and d. In some embodiments, one or both of the gRNAs is configured such that it comprises properties: a(i), b(viii), c, and e. In some embodiments, one or both of the gRNAs is configured such that it comprises properties: a(i), b(viii), c, d, and e. In some embodiments, one or both of the gRNAs is configured such that it comprises properties: a(i), b(ix), and c(i). In some embodiments, one or both of the gRNAs is configured such that it comprises properties: a(i), b(ix), and c(ii). In some embodiments, one or both of the gRNAs is configured such that it comprises properties: a(i), b(ix), c, and d. In some embodiments, one or both of the gRNAs is configured such that it comprises properties: a(i), b(ix), c, and e. In some embodiments, one or both of the gRNAs is configured such that it comprises properties: a(i), b(ix), c, d, and e. In some embodiments, one or both of the gRNAs is configured such that it comprises properties: a(i), b(x), and c(i). In some embodiments, one or both of the gRNAs is configured such that it comprises properties: a(i), b(x), and c(ii). In some embodiments, one or both of the gRNAs is configured such that it comprises properties: a(i), b(x), c, and d. In some embodiments, one or both of the gRNAs is configured such that it comprises properties: a(i), b(x), c, and e. In some embodiments, one or both of the gRNAs is configured such that it comprises properties: a(i), b(x), c, d, and e. In some embodiments, one or both of the gRNAs is configured such that it comprises properties: a(i), b(xi), and c(i). In some embodiments, one or both of the gRNAs is configured such that it comprises properties: a(i), b(xi), and c(ii). In some embodiments, one or both of the gRNAs is configured such that it comprises properties: a(i), b(xi), c, and d. In some embodiments, one or both of the gRNAs is configured such that it comprises properties: a(i), b(xi), c, and e. In some embodiments, one or both of the gRNAs is configured such that it comprises properties: a(i), b(xi), c, d, and e.

In some embodiments, the gRNAs are used with a Cas9 nickase molecule having HNH activity, e.g., a Cas9 molecule having the RuvC activity inactivated, e.g., a Cas9 molecule having a mutation at D10, e.g., the D10A mutation.

In some embodiments, the gRNAs are used with a Cas9 nickase molecule having RuvC activity, e.g., a Cas9 molecule having the HNH activity inactivated, e.g., a Cas9 molecule having a mutation at H840, e.g., a H840A. In some embodiments, the gRNAs are used with a Cas9 nickase molecule having RuvC activity, e.g., a Cas9 molecule having the HNH activity inactivated, e.g., a Cas9 molecule having a mutation at N863, e.g., N863A.

6) Functional Analysis of Agents for Gene Editing

Any of the Cas9 molecules, gRNA molecules, Cas9 molecule/gRNA molecule complexes, can be evaluated by art-known methods or as described herein. For example, exemplary methods for evaluating the endonuclease activity of Cas9 molecule are described, e.g., in Jinek et al., SCIENCE 2012, 337(6096):816-821.

G) Binding and Cleavage Assay: Testing the Endonuclease Activity of Cas9 Molecule

The ability of a Cas9 molecule/gRNA molecule complex to bind to and cleave a target nucleic acid can be evaluated in a plasmid cleavage assay. In this assay, synthetic or in vitro-transcribed gRNA molecule is pre-annealed prior to the reaction by heating to 95° C. and slowly cooling down to room temperature. Native or restriction digest-linearized plasmid DNA (300 ng (˜8 nM)) is incubated for 60 min at 37° C. with purified Cas9 protein molecule (50-500 nM) and gRNA (50-500 nM, 1:1) in a Cas9 plasmid cleavage buffer (20 mM HEPES pH 7.5, 150 mM KCl, 0.5 mM DTT, 0.1 mM EDTA) with or without 10 mM MgCl₂. The reactions are stopped with 5×DNA loading buffer (30% glycerol, 1.2% SDS, 250 mM EDTA), resolved by a 0.8 or 1% agarose gel electrophoresis and visualized by ethidium bromide staining. The resulting cleavage products indicate whether the Cas9 molecule cleaves both DNA strands, or only one of the two strands. For example, linear DNA products indicate the cleavage of both DNA strands. Nicked open circular products indicate that only one of the two strands is cleaved.

Alternatively, the ability of a Cas9 molecule/gRNA molecule complex to bind to and cleave a target nucleic acid can be evaluated in an oligonucleotide DNA cleavage assay. In this assay, DNA oligonucleotides (10 pmol) are radiolabeled by incubating with 5 units T4 polynucleotide kinase and ˜3-6 pmol (˜20-40 mCi) [γ-³²P]-ATP in 1×T4 polynucleotide kinase reaction buffer at 37° C. for 30 min, in a 50 μL reaction. After heat inactivation (65° C. for 20 min), reactions are purified through a column to remove unincorporated label. Duplex substrates (100 nM) are generated by annealing labeled oligonucleotides with equimolar amounts of unlabeled complementary oligonucleotide at 95° C. for 3 min, followed by slow cooling to room temperature. For cleavage assays, gRNA molecules are annealed by heating to 95° C. for 30 s, followed by slow cooling to room temperature. Cas9 (500 nM final concentration) is pre-incubated with the annealed gRNA molecules (500 nM) in cleavage assay buffer (20 mM HEPES pH 7.5, 100 mM KCl, 5 mM MgCl₂, 1 mM DTT, 5% glycerol) in a total volume of 9 μl. Reactions are initiated by the addition of 1 μl target DNA (10 nM) and incubated for 1 h at 37° C. Reactions are quenched by the addition of 20 μl of loading dye (5 mM EDTA, 0.025% SDS, 5% glycerol in formamide) and heated to 95° C. for 5 min. Cleavage products are resolved on 12% denaturing polyacrylamide gels containing 7 M urea and visualized by phosphorimaging. The resulting cleavage products indicate that whether the complementary strand, the non-complementary strand, or both, are cleaved.

One or both of these assays can be used to evaluate the suitability of any of the gRNA molecule or Cas9 molecule provided.

H) Binding Assay: Testing the Binding of Cas9 Molecule to Target DNA

Exemplary methods for evaluating the binding of Cas9 molecule to target DNA are described, e.g., in Jinek et al., SCIENCE 2012; 337(6096):816-821.

For example, in an electrophoretic mobility shift assay, target DNA duplexes are formed by mixing of each strand (10 nmol) in deionized water, heating to 95° C. for 3 min and slow cooling to room temperature. All DNAs are purified on 8% native gels containing 1×TBE. DNA bands are visualized by UV shadowing, excised, and eluted by soaking gel pieces in DEPC-treated H₂O. Eluted DNA is ethanol precipitated and dissolved in DEPC-treated H₂O. DNA samples are 5′ end labeled with [γ-32P]-ATP using T4 polynucleotide kinase for 30 min at 37° C. Polynucleotide kinase is heat denatured at 65° C. for 20 min, and unincorporated radiolabel is removed using a column. Binding assays are performed in buffer containing 20 mM HEPES pH 7.5, 100 mM KCl, 5 mM MgCl₂, 1 mM DTT and 10% glycerol in a total volume of 10 μl. Cas9 protein molecule is programmed with equimolar amounts of pre-annealed gRNA molecule and titrated from 100 μM to 1 μM. Radiolabeled DNA is added to a final concentration of 20 μM. Samples are incubated for 1 h at 37° C. and resolved at 4° C. on an 8% native polyacrylamide gel containing 1×TBE and 5 mM MgCl₂. Gels are dried and DNA visualized by phosphorimaging.

I) Techniques for Measuring Thermostability of Cas9/gRNA Complexes

The thermostability of Cas9-gRNA ribonucleoprotein (RNP) complexes can be detected by differential scanning fluorimetry (DSF) and other techniques. The thermostability of a protein can increase under favorable conditions such as the addition of a binding RNA molecule, e.g., a gRNA. Thus, information regarding the thermostability of a Cas9/gRNA complex is useful for determining whether the complex is stable.

J) Differential Scanning Flourimetry (DSF)

The thermostability of Cas9-gRNA ribonucleoprotein (RNP) complexes can be measured via DSF. RNP complexes, as described below, include a sequence of ribonucleotides, such as an RNA or a gRNA, and a protein, such as a Cas9 protein or variant thereof. This technique measures the thermostability of a protein, which can increase under favorable conditions such as the addition of a binding RNA molecule, e.g., a gRNA.

The assay can be applied in a number of ways. Exemplary protocols include, but are not limited to, a protocol to determine the desired solution conditions for RNP formation (assay 1, see below), a protocol to test the desired stoichiometric ratio of gRNA:Cas9 protein (assay 2, see below), a protocol to screen for effective gRNA molecules for Cas9 molecules, e.g., wild-type or mutant Cas9 molecules (assay 3, see below), and a protocol to examine RNP formation in the presence of target DNA (assay 4). In some embodiments, the assay is performed using two different protocols, one to test the best stoichiometric ratio of gRNA:Cas9 protein and another to determine the best solution conditions for RNP formation.

To determine the best solution to form RNP complexes, a 2 μM solution of Cas9 in water+10×SYPRO Orange® (Life Technologies cat #S-6650) and dispensed into a 384 well plate. An equimolar amount of gRNA diluted in solutions with varied pH and salt is then added. After incubating at room temperature for 10′ and brief centrifugation to remove any bubbles, a Bio-Rad CFX384™ Real-Time System C1000 Touch™ Thermal Cycler with the Bio-Rad CFX Manager software is used to run a gradient from 20° C. to 90° C. with a 1° increase in temperature every 10 seconds.

The second assay consists of mixing various concentrations of gRNA with 2 μM Cas9 in optimal buffer from assay 1 above and incubating at RT for 10′ in a 384 well plate. An equal volume of optimal buffer+10×SYPRO Orange® (Life Technologies cat #S-6650) is added and the plate sealed with Microseal® B adhesive (MSB-1001). Following brief centrifugation to remove any bubbles, a Bio-Rad CFX384™ Real-Time System C1000 Touch™ Thermal Cycler with the Bio-Rad CFX Manager software is used to run a gradient from 20° C. to 90° C. with a 1° increase in temperature every 10 seconds.

In the third assay, a Cas9 molecule (e.g., a Cas9 protein, e.g., a Cas9 variant protein) of interest is purified. A library of variant gRNA molecules is synthesized and resuspended to a concentration of 20 μM. The Cas9 molecule is incubated with the gRNA molecule at a final concentration of 1 μM each in a predetermined buffer in the presence of 5×SYPRO Orange® (Life Technologies cat #S-6650). After incubating at room temperature for 10 minutes and centrifugation at 2000 rpm for 2 minutes to remove any bubbles, a Bio-Rad CFX384™ Real-Time System C1000 Touch™ Thermal Cycler with the Bio-Rad CFX Manager software is used to run a gradient from 20° C. to 90° C. with an increase of 1° C. in temperature every 10 seconds.

In the fourth assay, a DSF experiment is performed with the following samples: Cas9 protein alone, Cas9 protein with gRNA, Cas9 protein with gRNA and target DNA, and Cas9 protein with target DNA. The order of mixing components is: reaction solution, Cas9 protein, gRNA, DNA, and SYPRO Orange. The reaction solution contains 10 mM HEPES pH 7.5, 100 mM NaCl, in the absence or presence of MgCl₂. Following centrifugation at 2000 rpm for 2 minutes to remove any bubbles, a Bio-Rad CFX384™ Real-Time System C1000 Touch™ Thermal Cycler with the Bio-Rad CFX Manager software is used to run a gradient from 20° C. to 90° C. with a 1° increase in temperature every 10 seconds.

3. Delivery of Agents for Genetic Disruption

In some embodiments, the targeted genetic disruption, e.g., DNA break, of the endogenous TGFBR2 locus (encoding TGFBRII) in humans is carried out by delivering or introducing one or more agent(s) capable of inducing a genetic disruption, e.g., Cas9 and/or gRNA components, to a cell, using any of a number of known delivery method or vehicle for introduction or transfer to cells, for example, using viral, e.g., lentiviral, delivery vectors, or any of the known methods or vehicles for delivering Cas9 molecules and gRNAs. Exemplary methods are described in, e.g., Wang et al. (2012) J. Immunother. 35(9): 689-701; Cooper et al. (2003) Blood. 101:1637-1644; Verhoeyen et al. (2009) Methods Mol Biol. 506: 97-114; and Cavalieri et al. (2003) Blood. 102(2): 497-505. In some embodiments, nucleic acid sequences encoding one or more components of one or more agent(s) capable of inducing a genetic disruption, e.g., DNA break, is introduced into the cells, e.g., by any methods for introducing nucleic acids into a cell described herein or known. In some embodiments, a vector encoding components of one or more agent(s) capable of inducing a genetic disruption such as a CRISPR guide RNA and/or a Cas9 enzyme can be delivered into the cell.

In some embodiments, the one or more agent(s) capable of inducing a genetic disruption, e.g., one or more agent(s) that is a Cas9/gRNA, is introduced into the cell as a ribonucleoprotein (RNP) complex. RNP complexes include a sequence of ribonucleotides, such as an RNA or a gRNA molecule, and a protein, such as a Cas9 protein or variant thereof. For example, the Cas9 protein is delivered as RNP complex that comprises a Cas9 protein and a gRNA molecule targeting the target sequence, e.g., using electroporation or other physical delivery method. In some embodiments, the RNP is delivered into the cell via electroporation or other physical means, e.g., particle gun, Calcium Phosphate transfection, cell compression or squeezing. In some embodiments, the RNP can cross the plasma membrane of a cell without the need for additional delivery agents (e.g., small molecule agents, lipids, etc.). In some embodiments, delivery of the one or more agent(s) capable of inducing genetic disruption, e.g., CRISPR/Cas9, as an RNP offers an advantage that the targeted disruption occurs transiently, e.g., in cells to which the RNP is introduced, without propagation of the agent to cell progenies. For example, delivery by RNP minimizes the agent from being inherited to its progenies, thereby reducing the chance of off-target genetic disruption in the progenies. In such cases, the genetic disruption and the integration of transgene can be inherited by the progeny cells, but without the agent itself, which may further introduce off-target genetic disruptions, being passed on to the progeny cells.

Agent(s) and components capable of inducing a genetic disruption, e.g., a Cas9 molecule and gRNA molecule, can be introduced into target cells in a variety of forms using a variety of delivery methods and formulations, as set forth in Tables 4 and 5, or methods described in, e.g., WO 2015/161276; US 2015/0056705, US 2016/0272999, US 2017/0211075; or US 2017/0016027. As described further herein, the delivery methods and formulations can be used to deliver template polynucleotides and/or other agents to the cell (such as those required for engineering the cells) in prior or subsequent steps of the methods described herein. When a Cas9 or gRNA component is encoded as DNA for delivery, the DNA may typically but not necessarily include a control region, e.g., comprising a promoter, to effect expression. Useful promoters for Cas9 molecule sequences include, e.g., CMV, EF-1α, EFS, MSCV, PGK, or CAG promoters. Useful promoters for gRNAs include, e.g., H1, EF-1α, tRNA or U6 promoters. Promoters with similar or dissimilar strengths can be selected to tune the expression of components. Sequences encoding a Cas9 molecule may comprise a nuclear localization signal (NLS), e.g., an SV40 NLS. In some embodiments a promoter for a Cas9 molecule or a gRNA molecule may be, independently, inducible, tissue specific, or cell specific. In some embodiments, an agent capable of inducing a genetic disruption is introduced RNP complexes.

TABLE 4
Exemplary Delivery Methods

| Elements | | |
|---|---|---|
| Cas9 | gRNA | |
| Mole- | mole- | |
| cule(s) | cule(s) | Comments |
| DNA | DNA | In this embodiment, a Cas9 molecule and a gRNA |
| | | are transcribed from DNA. In this embodiment, |
| | | they are encoded on separate molecules. |
| DNA | | In this embodiment, a Cas9 molecule and a gRNA |
| | | are transcribed from DNA, here from a single |
| | | molecule. |
| DNA | RNA | In this embodiment, a Cas9 molecule is transcribed |
| | | from DNA, and a gRNA is provided as in vitro |
| | | transcribed or synthesized RNA |
| mRNA | RNA | In this embodiment, a Cas9 molecule is translated |
| | | from in vitro transcribed mRNA, and a gRNA is |
| | | provided as in vitro transcribed or synthesized RNA. |
| mRNA | DNA | In this embodiment, a Cas9 molecule is translated |
| | | from in vitro transcribed mRNA, and a gRNA is |
| | | transcribed from DNA. |
| Protein | DNA | In this embodiment, a Cas9 molecule is provided |
| | | as a protein, and a gRNA is transcribed from DNA. |
| Protein | RNA | In this embodiment, a Cas9 molecule is provided |
| | | as a protein, and a gRNA is provided as |
| | | transcribed or synthesized RNA. |

TABLE 5
Comparison of Exemplary Delivery Methods

| | | Delivery | | | |
|---|---|---|---|---|---|
| | | into Non- | | | Type of |
| | | Dividing | Duration of | Genome | Molecule |
| Delivery Vector/Mode | | Cells | Expression | Integration | Delivered |
| Physical (e.g., electroporation, | | YES | Transient | NO | Nucleic |
| particle gun, Calcium Phosphate | | | | | Acids and |
| transfection, cell compression or | | | | | Proteins |
| squeezing) | | | | | |
| Viral | Retrovirus | NO | Stable | YES | RNA |
| | Lentivirus | YES | Stable | YES/NO | RNA |
| | | | | with | |
| | | | | modifications | |
| | Adenovirus | YES | Transient | NO | DNA |
| | Adeno-Associated | YES | Stable | NO | DNA |
| | Virus (AAV) | | | | |
| | Vaccinia Virus | YES | Very | NO | DNA |
| | | | Transient | | |
| | Herpes Simplex Virus | YES | Stable | NO | DNA |
| Non-Viral | Cationic Liposomes | YES | Transient | Depends on | Nucleic |
| | | | | what is | Acids and |
| | | | | delivered | Proteins |
| | Polymeric | YES | Transient | Depends on | Nucleic |
| | Nanoparticles | | | what is | Acids and |
| | | | | delivered | Proteins |
| Biological | Attenuated Bacteria | YES | Transient | NO | Nucleic |
| Non-Viral | | | | | Acids |
| Delivery | Engineered | YES | Transient | NO | Nucleic |
| Vehicles | Bacteriophages | | | | Acids |
| | Mammalian Virus- | YES | Transient | NO | Nucleic |
| | like Particles | | | | Acids |
| | Biological liposomes: | YES | Transient | NO | Nucleic |
| | Erythrocyte Ghosts | | | | Acids |
| | and Exosomes | | | | |

In some embodiments, DNA encoding Cas9 molecules and/or gRNA molecules, or RNP complexes comprising a Cas9 molecule and/or gRNA molecules, can be delivered into cells by known methods or as described herein. For example, Cas9-encoding and/or gRNA-encoding DNA can be delivered, e.g., by vectors (e.g., viral or non-viral vectors), non-vector based methods (e.g., using naked DNA or DNA complexes), or a combination thereof. In some embodiments, the polynucleotide containing the agent(s) and/or components thereof is delivered by a vector (e.g., viral vector/virus or plasmid). The vector may be any described herein.

In some aspects, a CRISPR enzyme (e.g. Cas9 nuclease) in combination with (and optionally complexed with) a guide sequence is delivered to the cell. For example, one or more elements of a CRISPR system is derived from a type I, type II, or type III CRISPR system. For example, one or more elements of a CRISPR system are derived from a particular organism comprising an endogenous CRISPR system, such as Streptococcus pyogenes, Staphylococcus aureus or Neisseria meningitides.

In some embodiments, a Cas9 nuclease (e.g., that encoded by mRNA from Staphylococcus aureus or from Streptococcus pyogenes, e.g. pCW-Cas9, Addgene #50661, Wang et al. (2014) Science, 3:343-80-4; or nuclease or nickase lentiviral vectors available from Applied Biological Materials (ABM; Canada) as Cat. No. K002, K003, K005 or K006) and a guide RNA specific to the target locus (e.g. TGFBR2 locus in humans) are introduced into cells.

In some embodiments, the polynucleotide containing the agent(s) and/or components thereof or RNP complex is delivered by a non-vector based method (e.g., using naked DNA or DNA complexes). For example, the DNA or RNA or proteins or combination thereof, e.g., ribonucleoprotein (RNP) complexes, can be delivered, e.g., by organically modified silica or silicate (Ormosil), electroporation, transient cell compression or squeezing (such as described in Lee, et al. (2012) Nano Lett 12: 6322-27, Kollmannsperger et al (2016) Nat Comm 7, 10372), gene gun, sonoporation, magnetofection, lipid-mediated transfection, dendrimers, inorganic nanoparticles, calcium phosphates, or a combination thereof.

In some embodiments, delivery via electroporation comprises mixing the cells with the Cas9- and/or gRNA-encoding DNA or RNP complex in a cartridge, chamber or cuvette and applying one or more electrical impulses of defined duration and amplitude. In some embodiments, delivery via electroporation is performed using a system in which cells are mixed with the Cas9- and/or gRNA-encoding DNA in a vessel connected to a device (e.g., a pump) which feeds the mixture into a cartridge, chamber or cuvette wherein one or more electrical impulses of defined duration and amplitude are applied, after which the cells are delivered to a second vessel.

In some embodiments, the delivery vehicle is a non-viral vector. In some embodiments, the non-viral vector is an inorganic nanoparticle. Exemplary inorganic nanoparticles include, e.g., magnetic nanoparticles (e.g., Fe₃MnO₂) and silica. The outer surface of the nanoparticle can be conjugated with a positively charged polymer (e.g., polyethylenimine, polylysine, polyserine) which allows for attachment (e.g., conjugation or entrapment) of payload. In some embodiments, the non-viral vector is an organic nanoparticle. Exemplary organic nanoparticles include, e.g., SNALP liposomes that contain cationic lipids together with neutral helper lipids which are coated with polyethylene glycol (PEG), and protamine-nucleic acid complexes coated with lipid. Exemplary lipids for gene transfer are shown below in Table 6.

TABLE 6
Lipids Used for Gene Transfer

| Lipid | Abbreviation | Feature |
|---|---|---|
| 1,2-Dioleoyl-sn-glycero-3-phosphatidylcholine | DOPC | Helper |
| 1,2-Dioleoyl-sn-glycero-3-phosphatidylethanolamine | DOPE | Helper |
| Cholesterol | | Helper |
| N-[1-(2,3-Dioleyloxy)prophyl]N,N,N-trimethylammonium chloride | DOTMA | Cationic |
| 1,2-Dioleoyloxy-3-trimethylammonium-propane | DOTAP | Cationic |
| Dioctadecylamidoglycylspermine | DOGS | Cationic |
| N-(3-Aminopropyl)-N,N-dimethyl-2,3-bis(dodecyloxy)-1- | GAP-DLRIE | Cationic |
| propanaminium bromide | | |
| Cetyltrimethylammonium bromide | CTAB | Cationic |
| 6-Lauroxyhexyl ornithinate | LHON | Cationic |
| 1-(2,3-Dioleoyloxypropyl)-2,4,6-trimethylpyridinium | 2Oc | Cationic |
| 2,3-Dioleyloxy-N-[2(sperminecarboxamido-ethyl]-N,N-dimethyl-1- | DOSPA | Cationic |
| propanaminium trifluoroacetate | | |
| 1,2-Dioleyl-3-trimethylammonium-propane | DOPA | Cationic |
| N-(2-Hydroxyethyl)-N,N-dimethyl-2,3-bis(tetradecyloxy)-1- | MDRIE | Cationic |
| propanaminium bromide | | |
| Dimyristooxypropyl dimethyl hydroxyethyl ammonium bromide | DMRI | Cationic |
| 3β-[N-(N′,N′-Dimethylaminoethane)-carbamoyl]cholesterol | DC-Chol | Cationic |
| Bis-guanidium-tren-cholesterol | BGTC | Cationic |
| 1,3-Diodeoxy-2-(6-carboxy-spermyl)-propylamide | DOSPER | Cationic |
| Dimethyloctadecylammonium bromide | DDAB | Cationic |
| Dioctadecylamidoglicylspermidin | DSL | Cationic |
| rac-[(2,3-Dioctadecyloxypropyl)(2-hydroxyethyl)]-dimethylammonium | CLIP-1 | Cationic |
| chloride | | |
| rac-[2(2,3-Dihexadecyloxypropyl- | CLIP-6 | Cationic |
| oxymethyloxy)ethyl]trimethylammonium bromide | | |
| Ethyldimyristoylphosphatidylcholine | EDMPC | Cationic |
| 1,2-Distearyloxy-N,N-dimethyl-3-aminopropane | DSDMA | Cationic |
| 1,2-Dimyristoyl-trimethylammonium propane | DMTAP | Cationic |
| O,O′-Dimyristyl-N-lysyl aspartate | DMKE | Cationic |
| 1,2-Distearoyl-sn-glycero-3-ethylphosphocholine | DSEPC | Cationic |
| N-Palmitoyl D-erythro-sphingosyl carbamoyl-spermine | CCS | Cationic |
| N-t-Butyl-N0-tetradecyl-3-tetradecylaminopropionamidine | diC14-amidine | Cationic |
| Octadecenolyoxy[ethyl-2-heptadecenyl-3 hydroxyethyl] imidazolinium | DOTIM | Cationic |
| chloride | | |
| N1-Cholesteryloxycarbonyl-3,7-diazanonane-1,9-diamine | CDAN | Cationic |
| 2-(3-[Bis(3-amino-propyl)-amino]propylamino)-N- | RPR209120 | Cationic |
| ditetradecylcarbamoylme-ethyl-acetamide | | |
| 1,2-dilinoleyloxy-3-dimethylaminopropane | DLinDMA | Cationic |
| 2,2-dilinoleyl-4-dimethylaminoethyl-[1,3]-dioxolane | DLin-KC2-DMA | Cationic |
| dilinoleyl-methyl-4-dimethylaminobutyrate | DLin-MC3-DMA | Cationic |

Exemplary polymers for gene transfer are shown below in Table 7.

TABLE 7
Polymers Used for Gene Transfer

| Polymer | Abbreviation |
|---|---|
| Poly(ethylene)glycol | PEG |
| Polyethylenimine | PEI |
| Dithiobis(succinimidylpropionate) | DSP |
| Dimethyl-3,3′-dithiobispropionimidate | DTBP |
| Poly(ethylene imine) biscarbamate | PEIC |
| Poly(L-lysine) | PLL |
| Histidine modified PLL | |
| Poly(N-vinylpyrrolidone) | PVP |
| Poly(propylenimine) | PPI |
| Poly(amidoamine) | PAMAM |
| Poly(amido ethylenimine) | SS-PAEI |
| Triethylenetetramine | TETA |
| Poly(β-aminoester) | |
| Poly(4-hydroxy-L-proline ester) | PHP |
| Poly(allylamine) | |
| Poly(α-[4-aminobutyl]-L-glycolic acid) | PAGA |
| Poly(D,L-lactic-co-glycolic acid) | PLGA |
| Poly(N-ethyl-4-vinylpyridinium bromide) | |
| Poly(phosphazene)s | PPZ |
| Poly(phosphoester)s | PPE |
| Poly(phosphoramidate)s | PPA |
| Poly(N-2-hydroxypropylmethacrylamide) | pHPMA |
| Poly(2-(dimethylamino)ethyl methacrylate) | pDMAEMA |
| Poly(2-aminoethyl propylene phosphate) | PPE-EA |
| Chitosan | |
| Galactosylated chitosan | |
| N-Dodacylated chitosan | |
| Histone | |
| Collagen | |
| Dextran-spermine | D-SPM |

In some embodiments, the vehicle has targeting modifications to increase target cell update of nanoparticles and liposomes, e.g., cell specific antigens, monoclonal antibodies, single chain antibodies, aptamers, polymers, sugars, and cell penetrating peptides. In some embodiments, the vehicle uses fusogenic and endosome-destabilizing peptides/polymers. In some embodiments, the vehicle undergoes acid-triggered conformational changes (e.g., to accelerate endosomal escape of the cargo). In some embodiments, a stimulus-cleavable polymer is used, e.g., for release in a cellular compartment. For example, disulfide-based cationic polymers that are cleaved in the reducing cellular environment can be used.

In some embodiments, the delivery vehicle is a biological non-viral delivery vehicle. In some embodiments, the vehicle is an attenuated bacterium (e.g., naturally or artificially engineered to be invasive but attenuated to prevent pathogenesis and expressing the transgene (e.g., Listeria monocytogenes, certain Salmonella strains, Bifidobacterium longum, and modified Escherichia coli), bacteria having nutritional and tissue-specific tropism to target specific cells, bacteria having modified surface proteins to alter target cell specificity). In some embodiments, the vehicle is a genetically modified bacteriophage (e.g., engineered phages having large packaging capacity, less immunogenicity, containing mammalian plasmid maintenance sequences and having incorporated targeting ligands). In some embodiments, the vehicle is a mammalian virus-like particle. For example, modified viral particles can be generated (e.g., by purification of the “empty” particles followed by ex vivo assembly of the virus with the desired cargo). The vehicle can also be engineered to incorporate targeting ligands to alter target tissue-specificity. In some embodiments, the vehicle is a biological liposome. For example, the biological liposome is a phospholipid-based particle derived from human cells (e.g., erythrocyte ghosts, which are red blood cells broken down into spherical structures derived from the subject (e.g., tissue targeting can be achieved by attachment of various tissue or cell-specific ligands), or secretory exosomes—subject-derived membrane-bound nanovescicles (30-100 nm) of endocytic origin (e.g., can be produced from various cell types and can therefore be taken up by cells without the need for targeting ligands).

In some embodiments, RNA encoding Cas9 molecules and/or gRNA molecules, can be delivered into cells, e.g., target cells described herein, by known methods or as described herein. For example, Cas9-encoding and/or gRNA-encoding RNA can be delivered, e.g., by microinjection, electroporation, transient cell compression or squeezing (such as described in Lee, et al. (2012) Nano Lett 12: 6322-27), lipid-mediated transfection, peptide-mediated delivery, e.g., cell-penetrating peptides, or a combination thereof.

In some embodiments, delivery via electroporation comprises mixing the cells with the RNA encoding Cas9 molecules and/or gRNA molecules in a cartridge, chamber or cuvette and applying one or more electrical impulses of defined duration and amplitude. In some embodiments, delivery via electroporation is performed using a system in which cells are mixed with the RNA encoding Cas9 molecules and/or gRNA molecules in a vessel connected to a device (e.g., a pump) which feeds the mixture into a cartridge, chamber or cuvette wherein one or more electrical impulses of defined duration and amplitude are applied, after which the cells are delivered to a second vessel.

In some embodiments, Cas9 molecules can be delivered into cells by known methods or as described herein. For example, Cas9 protein molecules can be delivered, e.g., by microinjection, electroporation, transient cell compression or squeezing (such as described in Lee, et al. (2012) Nano Lett 12: 6322-27), lipid-mediated transfection, peptide-mediated delivery, or a combination thereof. Delivery can be accompanied by DNA encoding a gRNA or by a gRNA.

In some embodiments, the one or more agent(s) capable of introducing a cleavage, e.g., a Cas9/gRNA system, is introduced into the cell as a ribonucleoprotein (RNP) complex. RNP complexes include a sequence of ribonucleotides, such as an RNA or a gRNA molecule, and a protein, such as a Cas9 protein or variant thereof. For example, the Cas9 protein is delivered as RNP complex that comprises a Cas9 protein and a gRNA molecule targeting the target sequence, e.g., using electroporation or other physical delivery method. In some embodiments, the RNP is delivered into the cell via electroporation or other physical means, e.g., particle gun, calcium phosphate transfection, cell compression or squeezing.

In some embodiments, delivery via electroporation comprises mixing the cells with the Cas9 molecules with or without gRNA molecules in a cartridge, chamber or cuvette and applying one or more electrical impulses of defined duration and amplitude. In some embodiments, delivery via electroporation is performed using a system in which cells are mixed with the Cas9 molecules with or without gRNA molecules in a vessel connected to a device (e.g., a pump) which feeds the mixture into a cartridge, chamber or cuvette wherein one or more electrical impulses of defined duration and amplitude are applied, after which the cells are delivered to a second vessel.

In some embodiments, delivery via electroporation comprises mixing the cells with the Cas9 molecules (e.g., eaCas9 molecules, eiCas9 molecules or eiCas9 fusion proteins) with or without gRNA molecules in a cartridge, chamber or cuvette and applying one or more electrical impulses of defined duration and amplitude. In some embodiments, delivery via electroporation is performed using a system in which cells are mixed with the Cas9 molecules (e.g., eaCas9 molecules, eiCas9 molecules or eiCas9 fusion proteins)

In some embodiments, the polynucleotide containing the agent(s) and/or components thereof is delivered by a combination of a vector and a non-vector based method. For example, a virosome comprises a liposome combined with an inactivated virus (e.g., HIV or influenza virus), which can result in more efficient gene transfer than either a viral or a liposomal method alone.

In some embodiments, more than one agent(s) or components thereof are delivered to the cell. For example, in some embodiments, agent(s) capable of inducing a genetic disruption of two or more locations in the genome, such as at two or more sites within a TGFBR2 locus (encoding TGFBRII), are delivered to the cell. In some embodiments, agent(s) and components thereof are delivered using one method. For example, in some embodiments, agent(s) for inducing a genetic disruption of the TGFBR2 locus are delivered as polynucleotides encoding the components for genetic disruption. In some embodiments, one polynucleotide can encode agents that target the TGFBR2 locus. In some embodiments, two or more different polynucleotides can encode the agents that target the TGFBR2 locus.

In some embodiments, the agents capable of inducing a genetic disruption can be delivered as ribonucleoprotein (RNP) complexes, and two or more different RNP complexes can be delivered together as a mixture, or separately.

In some embodiments, one or more nucleic acid molecules other than the one or more agent(s) capable of inducing a genetic disruption and/or component thereof, e.g., the Cas9 molecule component and/or the gRNA molecule component, such as a template polynucleotide for HDR-directed integration (such as any template polynucleotide described herein, e.g., in Section I.B), are delivered. In some embodiments, the nucleic acid molecule, e.g., template polynucleotide, is delivered at the same time as one or more of the components of the Cas system. In some embodiments, the nucleic acid molecule is delivered before or after (e.g., less than about 1 minute, 5 minutes, 10 minutes, 15 minutes, 30 minutes, 1 hour, 2 hours, 3 hours, 6 hours, 9 hours, 12 hours, 1 day, 2 days, 3 days, 1 week, 2 weeks, or 4 weeks) one or more of the components of the Cas system are delivered. In some embodiments, the nucleic acid molecule, e.g., template polynucleotide, is delivered by a different means from one or more of the components of the Cas system, e.g., the Cas9 molecule component and/or the gRNA molecule component. The nucleic acid molecule, e.g., template polynucleotide, can be delivered by any of the delivery methods described herein. For example, the nucleic acid molecule, e.g., template polynucleotide, can be delivered by a viral vector, e.g., a retrovirus or a lentivirus, and the Cas9 molecule component and/or the gRNA molecule component can be delivered by electroporation. In some embodiments, the nucleic acid molecule, e.g., template polynucleotide, includes one or more exogenous sequences, e.g., sequences that encode a recombinant receptor or a portion thereof and/or other exogenous gene nucleic acid sequences.

### B. Targeted Integration Via Homology-Directed Repair (HDR)

In some aspects, the provided embodiments involve targeted integration of a specific part of a polynucleotide, such as the part of a template polynucleotide containing transgene sequences encoding a recombinant receptor or a portion thereof, at a particular location (such as target site or target location) in the genome at the endogenous TGFBR2 locus encoding TGFBRII. In some aspects, homology-directed repair (HDR) can mediate the site specific integration of the transgene sequences at the target site. In some embodiments, the presence of a genetic disruption (e.g., a DNA break, such as described in Section I.A) and a template polynucleotide containing one or more homology arms (e.g., containing nucleic acid sequences homologous sequences surrounding the genetic disruption) can induce or direct HDR, with homologous sequences acting as a template for DNA repair. Based on homology between the endogenous gene sequence surrounding the genetic disruption and the 5′ and/or 3′ homology arms included in the template polynucleotide, cellular DNA repair machinery can use the template polynucleotide to repair the DNA break and resynthesize (e.g., copy) genetic information at the site of the genetic disruption, thereby effectively inserting or integrating the transgene sequences in the template polynucleotide at or near the site of the genetic disruption. In some embodiments, the genetic disruption at an endogenous TGFBR2 locus, can be generated by any of the methods for generating a targeted genetic disruption described herein, for example, in Section I.A.

Also provided are polynucleotides, e.g., template polynucleotides described herein, and kits that include such polynucleotides. In some embodiments, the provided polynucleotides and/or kits can be employed in the methods described herein, e.g., involving HDR, to target transgene sequences encoding a recombinant receptor or a portion thereof at the endogenous TGFBR2 locus.

In some embodiments, the template polynucleotide is or comprises a polynucleotide containing a transgene, such as exogenous or heterologous nucleic acid sequences, encoding a recombinant receptor or a portion thereof (e.g., one or more region(s) or domain(s) of the recombinant receptor), and homology sequences (e.g., homology arms) that are homologous to sequences at or near the endogenous genomic site at the endogenous TGFBR2 locus. In some aspects, the transgene sequences in the template polynucleotide comprise sequence of nucleotides encoding a recombinant receptor or a portion thereof. In some aspects, upon targeted integration of the transgene sequences, the TGFBR2 locus in the engineered cell is modified such that the modified TGFBR2 locus contains the transgene sequences encoding a recombinant receptor, e.g., a chimeric antigen receptor (CAR). In some aspects, the modified TGFBR2 locus encodes a dominant negative form of the TGFBRII polypeptide and a recombinant receptor, e.g., CAR.

In some aspects, the template polynucleotide is introduced as a linear DNA fragment or comprised in a vector. In some aspects, the step for inducing genetic disruption and the step for targeted integration (e.g., by introduction of the template polynucleotide) are performed simultaneously or sequentially.

1. Homology-Directed Repair (HDR)

In some embodiments, homology-directed repair (HDR) can be utilized for targeted integration or insertion of one or more nucleic acid sequences, e.g., transgene sequences encoding a recombinant receptor or a portion thereof, at one or more target site(s) in the genome at a TGFBR2 locus. In some embodiments, the nuclease-induced HDR can be used to alter a target sequence, integrate transgene sequences at a particular target location, and/or to edit or repair a mutation in a particular target gene.

Alteration of nucleic acid sequences at the target site can occur by HDR with an exogenously provided polynucleotide, e.g., template polynucleotide (also referred to as “donor polynucleotide” or “template sequence”). For example, the template polynucleotide provides for alteration of the target sequence, such as insertion of the transgene sequences contained within the template polynucleotide. In some embodiments, a plasmid or a vector can be used as a template for homologous recombination. In some embodiments, a linear DNA fragment can be used as a template for homologous recombination. In some embodiments, a single stranded template polynucleotide can be used as a template for alteration of the target sequence by alternate methods of homology directed repair (e.g., single strand annealing) between the target sequence and the template polynucleotide. Template polynucleotide-effected alteration of a target sequence depends on cleavage by a nuclease, e.g., a targeted nuclease such as CRISPR/Cas9. Cleavage by the nuclease can comprise a double strand break or two single strand breaks.

In some embodiments, “recombination” includes a process of exchange of genetic information between two polynucleotides. In some embodiments, “homologous recombination (HR)” includes a specialized form of such exchange that takes place, for example, during repair of double-strand breaks in cells via homology-directed repair mechanisms. This process requires nucleotide sequence homology, uses a template polynucleotide to template repair of a target DNA (i.e., the one that experienced the double-strand break, such as target site in the endogenous gene), and is variously known as “non-crossover gene conversion” or “short tract gene conversion,” because it leads to the transfer of genetic information from the template polynucleotide to the target. In some embodiments, such transfer can involve mismatch correction of heteroduplex DNA that forms between the broken target and the template polynucleotide, and/or “synthesis-dependent strand annealing,” in which the template polynucleotide is used to resynthesize genetic information that will become part of the target, and/or related processes. Such specialized HR often results in an alteration of the sequence of the target molecule such that part or all of the sequence of the template polynucleotide is incorporated into the target polynucleotide.

In some embodiments, a portion of the polynucleotide, such as the template polynucleotide, e.g., polynucleotide containing transgene, is integrated into the genome of a cell via homology-independent mechanisms. The methods comprise creating a double-stranded break (DSB) in the genome of a cell and cleaving the template polynucleotide molecule using a nuclease, such that the template polynucleotide is integrated at the site of the DSB. In some embodiments, the template polynucleotide is integrated via non-homology dependent methods (e.g., NHEJ). Upon in vivo cleavage the template polynucleotides can be integrated in a targeted manner into the genome of a cell at the location of a DSB. The template polynucleotide can include one or more of the same target sites for one or more of the nucleases used to create the DSB. Thus, the template polynucleotide may be cleaved by one or more of the same nucleases used to cleave the endogenous gene into which integration is desired. In some embodiments, the template polynucleotide includes different nuclease target sites from the nucleases used to induce the DSB. As described herein, the genetic disruption of the target site or target position can be created by any know methods or any methods described herein, such as ZFNs, TALENs, CRISPR/Cas9 system, or TtAgo nucleases.

In some embodiments, DNA repair mechanisms can be induced by a nuclease after (1) a single double-strand break, (2) two single strand breaks, (3) two double stranded breaks with a break occurring on each side of the target site, (4) one double stranded break and two single strand breaks with the double strand break and two single strand breaks occurring on each side of the target site (5) four single stranded breaks with a pair of single stranded breaks occurring on each side of the target site, or (6) one single stranded break. In some embodiments, a single-stranded template polynucleotide is used and the target site can be altered by alternative HDR.

Template polynucleotide-effected alteration of a target site depends on cleavage by a nuclease molecule. Cleavage by the nuclease can comprise a nick, a double strand break, or two single strand breaks, e.g., one on each strand of the DNA at the target site. After introduction of the breaks on the target site, resection occurs at the break ends resulting in single stranded overhanging DNA regions.

In canonical HDR, a double-stranded template polynucleotide is introduced, comprising homologous sequence to the target site that will either be directly incorporated into the target site or used as a template to insert the transgene or correct the sequence of the target site. After resection at the break, repair can progress by different pathways, e.g., by the double Holliday junction model (or double strand break repair, DSBR, pathway) or the synthesis-dependent strand annealing (SDSA) pathway.

In the double Holliday junction model, strand invasion by the two single stranded overhangs of the target site to the homologous sequences in the template polynucleotide occurs, resulting in the formation of an intermediate with two Holliday junctions. The junctions migrate as new DNA is synthesized from the ends of the invading strand to fill the gap resulting from the resection. The end of the newly synthesized DNA is ligated to the resected end, and the junctions are resolved, resulting in the insertion at the target site, e.g., insertion of the transgene in template polynucleotide. Crossover with the template polynucleotide may occur upon resolution of the junctions.

In the SDSA pathway, only one single stranded overhang invades the template polynucleotide and new DNA is synthesized from the end of the invading strand to fill the gap resulting from resection. The newly synthesized DNA then anneals to the remaining single stranded overhang, new DNA is synthesized to fill in the gap, and the strands are ligated to produce the modified DNA duplex.

In alternative HDR, a single strand template polynucleotide, e.g., template polynucleotide, is introduced. A nick, single strand break, or double strand break at the target site, for altering a desired target site, is mediated by a nuclease molecule, and resection at the break occurs to reveal single stranded overhangs. Incorporation of the sequence of the template polynucleotide to correct or alter the target site of the DNA typically occurs by the SDSA pathway, as described herein.

“Alternative HDR”, or alternative homology-directed repair, in some embodiments, refers to the process of repairing DNA damage using a homologous nucleic acid (e.g., an endogenous homologous sequence, e.g., a sister chromatid, or an exogenous nucleic acid, e.g., a template polynucleotide). Alternative HDR is distinct from canonical HDR in that the process utilizes different pathways from canonical HDR, and can be inhibited by the canonical HDR mediators, RAD51 and BRCA2. Also, alternative HDR uses a single-stranded or nicked homologous nucleic acid for repair of the break. “Canonical HDR”, or canonical homology-directed repair, in some embodiments, refers to the process of repairing DNA damage using a homologous nucleic acid (e.g., an endogenous homologous sequence, e.g., a sister chromatid, or an exogenous nucleic acid, e.g., a template nucleic acid). Canonical HDR typically acts when there has been significant resection at the double strand break, forming at least one single stranded portion of DNA In a normal cell, HDR typically involves a series of steps such as recognition of the break, stabilization of the break, resection, stabilization of single stranded DNA, formation of a DNA crossover intermediate, resolution of the crossover intermediate, and ligation. The process requires RAD51 and BRCA2 and the homologous nucleic acid is typically double-stranded. Unless indicated otherwise, the term “HDR” in some embodiments encompasses canonical HDR and alternative HDR.

In some embodiments, double strand cleavage is effected by a nuclease, e.g., a Cas9 molecule having cleavage activity associated with an HNH-like domain and cleavage activity associated with a RuvC-like domain, e.g., an N-terminal RuvC-like domain, e.g., a wild type Cas9. Such embodiments require only a single gRNA.

In some embodiments, one single strand break, or nick, is effected by a nuclease molecule having nickase activity, e.g., a Cas9 nickase. A nicked DNA at the target site can be a substrate for alternative HDR.

In some embodiments, two single strand breaks, or nicks, are effected by a nuclease, e.g., Cas9 molecule, having nickase activity, e.g., cleavage activity associated with an HNH-like domain or cleavage activity associated with an N-terminal RuvC-like domain. Such embodiments usually require two gRNAs, one for placement of each single strand break. In some embodiments, the Cas9 molecule having nickase activity cleaves the strand to which the gRNA hybridizes, but not the strand that is complementary to the strand to which the gRNA hybridizes. In some embodiments, the Cas9 molecule having nickase activity does not cleave the strand to which the gRNA hybridizes, but rather cleaves the strand that is complementary to the strand to which the gRNA hybridizes. In some embodiments, the nickase has HNH activity, e.g., a Cas9 molecule having the RuvC activity inactivated, e.g., a Cas9 molecule having a mutation at D10, e.g., the D10A mutation. D10A inactivates RuvC; therefore, the Cas9 nickase has (only) HNH activity and will cut on the strand to which the gRNA hybridizes (e.g., the complementary strand, which does not have the NGG PAM on it). In some embodiments, a Cas9 molecule having an H840, e.g., an H840A, mutation can be used as a nickase. H840A inactivates HNH; therefore, the Cas9 nickase has (only) RuvC activity and cuts on the non-complementary strand (e.g., the strand that has the NGG PAM and whose sequence is identical to the gRNA). In some embodiments, the Cas9 molecule is an N-terminal RuvC-like domain nickase, e.g., the Cas9 molecule comprises a mutation at N863, e.g., N863A.

In some embodiments, in which a nickase and two gRNAs are used to position two single strand nicks, one nick is on the + strand and one nick is on the—strand of the target DNA. The PAMs are outwardly facing. The gRNAs can be selected such that the gRNAs are separated by, from about 0-50, 0-100, or 0-200 nucleotides. In some embodiments, there is no overlap between the target sequences that are complementary to the targeting domains of the two gRNAs. In some embodiments, the gRNAs do not overlap and are separated by as much as 50, 100, or 200 nucleotides. In some embodiments, the use of two gRNAs can increase specificity, e.g., by decreasing off-target binding (Ran et al., Cell 2013).

In some embodiments, a single nick can be used to induce HDR, e.g., alternative HDR. It is contemplated herein that a single nick can be used to increase the ratio of HR to NHEJ at a given cleavage site, such as target site. In some embodiments, a single strand break is formed in the strand of the DNA at the target site to which the targeting domain of said gRNA is complementary. In some embodiments, a single strand break is formed in the strand of the DNA at the target site other than the strand to which the targeting domain of said gRNA is complementary.

In some embodiments, other DNA repair pathways such as single strand annealing (SSA), single-stranded break repair (SSBR), mismatch repair (MMR), base excision repair (BER), nucleotide excision repair (NER), interstrand cross-link (ICL), translesion synthesis (TLS), error-free post replication repair (PRR) can be employed by the cell to repair a double-stranded or single-stranded break created by the nucleases.

Targeted integration results in the transgene, e.g., sequences between the homology arms, being integrated into a TGFBR2 locus in the genome. The transgene may be integrated anywhere at or near one of the at least one target site(s) or site in the genome. In some embodiments, the transgene is integrated at or near one of the at least one target site(s), for example, within 300, 250, 200, 150, 100, 50, 10, 5, 4, 3, 2, 1 or fewer base pairs upstream or downstream of the site of cleavage, such as within 100, 50, 10, 5, 4, 3, 2, 1 base pairs of either side of the target site, such as within 50, 10, 5, 4, 3, 2, 1 base pairs of either side of the target site. In some embodiments, the integrated sequence comprising the transgene does not include any vector sequences (e.g., viral vector sequences). In some embodiments, the integrated sequence includes a portion of the vector sequences (e.g., viral vector sequences).

The double strand break or single strand break (such as target site) in one of the strands should be sufficiently close to the target integration site, e.g., site for targeted integration, such that an alteration is produced in the desired region, such as insertion of transgene or correction of a mutation occurs. In some embodiments, the distance is not more than 10, 25, 50, 100, 200, 300, 350, 400 or 500 nucleotides. In some embodiments, it is believed that the break should be sufficiently close to the target integration site such that the break is within the region that is subject to exonuclease-mediated removal during end resection. In some embodiments, the targeting domain is configured such that a cleavage event, e.g., a double strand or single strand break, is positioned within 1, 2, 3, 4, 5, 10, 15, 20, 25, 30, 35, 40, 45, 50, 60, 70, 80, 90, 100, 150, 200, 300, 350, 400 or 500 nucleotides of the region desired to be altered, e.g., site for targeted insertion. The break, e.g., a double strand or single strand break, can be positioned upstream or downstream of the region desired to be altered, e.g., site for targeted insertion. In some embodiments, a break is positioned within the region desired to be altered, e.g., within a region defined by at least two mutant nucleotides. In some embodiments, a break is positioned immediately adjacent to the region desired to be altered, e.g., immediately upstream or downstream of target integration site.

In some embodiments, a single strand break is accompanied by an additional single strand break, positioned by a second gRNA molecule. For example, the targeting domains are configured such that a cleavage event, e.g., the two single strand breaks, are positioned within 1, 2, 3, 4, 5, 10, 15, 20, 25, 30, 35, 40, 45, 50, 60, 70, 80, 90, 100, 150, 200, 300, 350, 400 or 500 nucleotides of a target integration site. In some embodiments, the first and second gRNA molecules are configured such, that when guiding a Cas9 nickase, a single strand break will be accompanied by an additional single strand break, positioned by a second gRNA, sufficiently close to one another to result in alteration of the desired region. In some embodiments, the first and second gRNA molecules are configured such that a single strand break positioned by said second gRNA is within 10, 20, 30, 40, or 50 nucleotides of the break positioned by said first gRNA molecule, e.g., when the Cas9 is a nickase. In some embodiments, the two gRNA molecules are configured to position cuts at the same position, or within a few nucleotides of one another, on different strands, e.g., essentially mimicking a double strand break.

In some embodiments, in which a gRNA (unimolecular (or chimeric) or modular gRNA) and Cas9 nuclease induce a double strand break for the purpose of inducing HDR to mediated insertion of transgene or correction, the cleavage site, such as target site, is between 0 to 200 bp (e.g., 0 to 175, 0 to 150, 0 to 125, 0 to 100, 0 to 75, 0 to 50, 0 to 25, 25 to 200, 25 to 175, 25 to 150, 25 to 125, 25 to 100, 25 to 75, 25 to 50, 50 to 200, 50 to 175, 50 to 150, 50 to 125, 50 to 100, 50 to 75, 75 to 200, 75 to 175, 75 to 150, 75 to 125, 75 to 100 bp) away from the target integration site. In some embodiments, the cleavage site, such as target site, is between 0 to 100 bp (e.g., 0 to 75, 0 to 50, 0 to 25, 25 to 100, 25 to 75, 25 to 50, 50 to 100, 50 to 75 or 75 to 100 bp) away from the site for targeted integration.

In some embodiments, one can promote HDR by using nickases to generate a break with overhangs. In some embodiments, the single stranded nature of the overhangs can enhance the cell's likelihood of repairing the break by HDR as opposed to, e.g., NHEJ.

Specifically, in some embodiments, HDR is promoted by selecting a first gRNA that targets a first nickase to a first target site, and a second gRNA that targets a second nickase to a second target site which is on the opposite DNA strand from the first target site and offset from the first nick. In some embodiments, the targeting domain of a gRNA molecule is configured to position a cleavage event sufficiently far from a preselected nucleotide, e.g., the nucleotide of a coding region, such that the nucleotide is not altered. In some embodiments, the targeting domain of a gRNA molecule is configured to position an intronic cleavage event sufficiently far from an intron/exon border, or naturally occurring splice signal, to avoid alteration of the exonic sequence or unwanted splicing events. In some embodiments, the targeting domain of a gRNA molecule is configured to position in an early exon, to allow in-frame integration of the transgene sequence at or near one of the at least one target site(s).

In some embodiments, a double strand break can be accompanied by an additional double strand break, positioned by a second gRNA molecule. In some embodiments, a double strand break can be accompanied by two additional single strand breaks, positioned by a second gRNA molecule and a third gRNA molecule. In some embodiments, two gRNAs, e.g., independently, unimolecular (or chimeric) or modular gRNA, are configured to position a double-strand break on both sides of a target integration site, e.g., site for targeted integration.

2. Template Polynucleotide

In some embodiments, a template polynucleotide, e.g., a polynucleotide containing a transgene, such as exogenous or heterologous nucleic acid sequences, that includes a sequence of nucleotides encoding one or more chains of a recombinant receptor, a chimeric receptor or a portion thereof, and homology sequences (e.g., homology arms) that are homologous to sequences at or near the endogenous genomic site for targeted integration, can be employed molecules and machinery involved in cellular DNA repair processes, such as homologous recombination, as a repair template. In some aspects, a template polynucleotide having homology with sequences at or near one or more target site(s) in the endogenous DNA can be used to alter the structure of a target DNA, such as target site at the endogenous TGFBR2 locus, for targeted insertion of the transgenic, heterologous or exogenous sequences, e.g., exogenous nucleic acid sequences encoding the one or more chains of a recombinant receptor or portion thereof. Also provided are polynucleotides, e.g., template polynucleotides, for use in the methods provided herein, e.g., as templates for homology directed repair (HDR) mediated targeted integration of the transgene sequences. In some embodiments, the polynucleotide includes a nucleic acid sequence, such as a transgene, encoding one or more chains of a recombinant receptor or a portion thereof; and one or more homology arm(s) linked to the nucleic acid sequence, wherein the one or more homology arm(s) comprise a sequence homologous to one or more region(s) of an open reading frame of a TGFBR2 locus.

In some embodiments, the template polynucleotide contains one or more homology sequences (e.g., homology arms) linked to and/or flanking the transgene (exogenous or heterologous nucleic acids sequences) that includes a sequence of nucleotides encoding the one or more chains of a recombinant receptor or portion thereof. In some embodiments, the homology sequences are used to target the exogenous sequences at the endogenous TGFBR2 locus. In some embodiments, the template polynucleotide includes nucleic acid sequences, such as transgene sequences, between the homology arms, for insertion or integration into the genome of a cell. The transgene in the template polynucleotide may comprise one or more sequences encoding a functional polypeptide (e.g., a cDNA), with or without a promoter or other regulatory elements.

In some embodiments, a template polynucleotide is a nucleic acid sequence which can be used in conjunction with one or more agent(s) capable of introducing a genetic disruption, to alter the structure of a target site. In some embodiments, the template polynucleotide alters the structure of the target site, e.g., insertion of transgene, by a homology directed repair event.

In some embodiments, the template polynucleotide alters the sequence of the target site, e.g., results in insertion or integration of the transgene sequences between the homology arms, into the genome of the cell. In some aspects, targeted integration results in an in-frame integration of the coding portion of the transgene sequences with one or more exons of the open reading frame of the endogenous TGFBR2 locus, e.g., in-frame with the adjacent exon at the integration site. For example, in some cases, the in-frame integration results in a portion of the endogenous open reading frame and the recombinant receptor or portion thereof to be expressed, optionally separated by a multicistronic element, such as a 2A element. Thus, the modified TGFBR2 locus can express a polypeptide containing a portion of TGFBRII and the recombinant receptor or portion thereof, which can be separated into 2 different polypeptides by virtue of the multicistronic element.

In some embodiments, the template polynucleotide includes sequences that correspond to or is homologous to a site on the target sequence that is cleaved, e.g., by one or more agent(s) capable of introducing a genetic disruption. In some embodiments, the template polynucleotide includes sequences that correspond to or is homologous to both, a first site on the target sequence that is cleaved in a first agent capable of introducing a genetic disruption, and a second site on the target sequence that is cleaved in a second agent capable of introducing a genetic disruption.

In some embodiments, a template polynucleotide comprises the following components: [5′ homology arm]-[transgene sequences (exogenous or heterologous nucleic acid sequences, e.g., encoding one or more chains of a recombinant receptor or a portion thereof)]-[3′ homology arm]. The homology arms provide for recombination into the chromosome, thus effectively inserting or integrating the transgene, e.g., that encodes a the recombinant receptor or portion thereof, into the genomic DNA at or near the cleavage site, such as target site(s). In some embodiments, the homology arms flank the sequences at the target site of genetic disruption.

In some embodiments, the template polynucleotide is double stranded. In some embodiments, the template polynucleotide is single stranded. In some embodiments, the template polynucleotide comprises a single stranded portion and a double stranded portion. In some embodiments, the template polynucleotide is comprised in a vector. In some embodiments, the template polynucleotide is DNA. In some embodiments, the template polynucleotide is RNA. In some embodiments, the template polynucleotide is double stranded DNA. In some embodiments, the template polynucleotide is single stranded DNA. In some embodiments, the template polynucleotide is double stranded RNA. In some embodiments, the template polynucleotide is single stranded RNA. In some embodiments, the template polynucleotide comprises a single stranded portion and a double stranded portion. In some embodiments, the template polynucleotide is comprised in a vector.

In certain embodiments, the polynucleotide, e.g., template polynucleotide contains and/or includes a transgene encoding one or more chains of a recombinant receptor, e.g., a CAR or a portion thereof. In particular embodiments, the transgene is targeted at a target site(s) that is within an endogenous gene, locus, or open reading frame that encodes the TGFBRII. In some embodiments, the transgene is targeted for integration within the endogenous TGFBR2 open reading frame, such as to result in a coding sequence that encodes a dominant negative form of the TGFBRII polypeptide.

Polynucleotides for insertion can also be referred to as “transgene” or “exogenous sequences” or “donor” polynucleotides or molecules. The template polynucleotide can be DNA, single-stranded and/or double-stranded and can be introduced into a cell in linear or circular form. The template polynucleotide can be DNA, single-stranded and/or double-stranded and can be introduced into a cell in linear or circular form. The template polynucleotide can be RNA single-stranded and/or double-stranded and can be introduced as a RNA molecule (e.g., part of an RNA virus). See also, U.S. Patent Pub. Nos. 20100047805 and 20110207221. The template polynucleotide can also be introduced in DNA form, which may be introduced into the cell in circular or linear form. If introduced in linear form, the ends of the template polynucleotide can be protected (e.g., from exonucleolytic degradation) by known methods. For example, one or more dideoxynucleotide residues are added to the 3′ terminus of a linear molecule and/or self-complementary oligonucleotides are ligated to one or both ends. See, for example, Chang et al. (1987) Proc. Natl. Acad. Sci. USA 84:4959-4963; Nehls et al. (1996) Science 272:886-889. Additional methods for protecting exogenous polynucleotides from degradation include, but are not limited to, addition of terminal amino group(s) and the use of modified internucleotide linkages such as, for example, phosphorothioates, phosphoramidates, and O-methyl ribose or deoxyribose residues. If introduced in double-stranded form, the template polynucleotide may include one or more nuclease target site(s), for example, nuclease target sites flanking the transgene to be integrated into the cell's genome. See, e.g., U.S. Patent Pub. No. 20130326645.

In some embodiments, the double-stranded template polynucleotide includes sequences (also referred to as transgene) greater than 1 kb in length, for example between 2 and 200 kb, between 2 and 10 kb (or any value therebetween). The double-stranded template polynucleotide also includes at least one nuclease target site, for example. In some embodiments, the template polynucleotide includes at least 2 target sites, for example for a pair of ZFNs or TALENs. Typically, the nuclease target sites are outside the transgene sequences, for example, 5′ and/or 3′ to the transgene sequences, for cleavage of the transgene. The nuclease cleavage site(s), such as target sites(s), may be for any nuclease(s). In some embodiments, the nuclease target site(s) contained in the double-stranded template polynucleotide are for the same nuclease(s) used to cleave the endogenous target into which the cleaved template polynucleotide is integrated via homology-independent methods.

In some embodiments, the template polynucleotide is a single stranded nucleic acid. In some embodiments, the template polynucleotide is a double stranded nucleic acid. In some embodiments, the template polynucleotide comprises a nucleotide sequence, e.g., of one or more nucleotides, that will be added to or will template a change in the target DNA. In some embodiments, the template polynucleotide comprises a nucleotide sequence that may be used to modify the target site. In some embodiments, the template polynucleotide comprises a nucleotide sequence, e.g., of one or more nucleotides, that corresponds to wild type sequence of the target DNA, e.g., of the target site.

In some embodiments, the template polynucleotide is linear double stranded DNA. The length may be, e.g., about 200 to about 5000 base pairs, e.g., about 200, 300, 400, 500, 600, 700, 800, 900, 1000, 1200, 1400, 1600, 1800, 2000, 2500, 3000, 4000 or 5000 base pairs. The length may be, e.g., at least 200, 300, 400, 500, 600, 700, 800, 900, 1000, 1200, 1400, 1600, 1800, 2000, 2500, 3000, 4000 or 5000 base pairs. In some embodiments, the length is no greater than 200, 300, 400, 500, 600, 700, 800, 900, 1000, 1200, 1400, 1600, 1800, 2000, 2500, 3000, 4000 or 5000 base pairs. In some embodiments, a double stranded template polynucleotide has a length of about 160 base pairs, e.g., about 200 to 4000, 300 to 3500, 400 to 3000, 500 to 2500, 600 to 2000, 700 to 1900, 800 to 1800, 900 to 1700, 1000 to 1600, 1100 to 1500 or 1200 to 1400 base pairs.

The transgene contained on the template polynucleotide described herein may be isolated from plasmids, cells or other sources using known standard techniques such as PCR. Template polynucleotide for use can include varying types of topology, including circular supercoiled, circular relaxed, linear and the like. Alternatively, they may be chemically synthesized using standard oligonucleotide synthesis techniques. In addition, template polynucleotides may be methylated or lack methylation. Template polynucleotides may be in the form of bacterial or yeast artificial chromosomes (BACs or YACs).

The template polynucleotide can be linear single stranded DNA In some embodiments, the template polynucleotide is (i) linear single stranded DNA that can anneal to the nicked strand of the target DNA, (ii) linear single stranded DNA that can anneal to the intact strand of the target DNA, (iii) linear single stranded DNA that can anneal to the transcribed strand of the target DNA, (iv) linear single stranded DNA that can anneal to the non-transcribed strand of the target DNA, or more than one of the preceding.

The length may be, e.g., about 200 to 5000 nucleotides, e.g., about 200, 300, 400, 500, 600, 700, 800, 900, 1000, 1200, 1400, 1600, 1800, 2000, 2500, 3000, 4000 or 5000 nucleotides. The length may be, e.g., at least 200, 300, 400, 500, 600, 700, 800, 900, 1000, 1200, 1400, 1600, 1800, 2000, 2500, 3000, 4000 or 5000 nucleotides. In some embodiments, the length is no greater than 200, 300, 400, 500, 600, 700, 800, 900, 1000, 1200, 1400, 1600, 1800, 2000, 2500, 3000, 4000 or 5000 nucleotides. In some embodiments, a single stranded template polynucleotide has a length of about 160 nucleotides, e.g., about 200 to 4000, 300 to 3500, 400 to 3000, 500 to 2500, 600 to 2000, 700 to 1900, 800 to 1800, 900 to 1700, 1000 to 1600, 1100 to 1500 or 1200 to 1400 nucleotides.

In some embodiments, the template polynucleotide is circular double stranded DNA, e.g., a plasmid. In some embodiments, the template polynucleotide comprises about 500 to 1000 base pairs of homology on either side of the transgene and/or the target site. In some embodiments, the template polynucleotide comprises about 10, 20, 30, 40, 50, 100, 200, 300, 400, 500, 600, 700, 800, 900, 1000, 1500, or 2000 base pairs of homology 5′ of the target site or transgene, 3′ of the target site or transgene, or both 5′ and 3′ of the target site or transgene. In some embodiments, the template polynucleotide comprises at least 10, 20, 30, 40, 50, 100, 200, 300, 400, 500, 600, 700, 800, 900, 1000, 1500, or 2000 base pairs of homology 5′ of the target site or transgene, 3′ of the target site or transgene, or both 5′ and 3′ of the target site or transgene. In some embodiments, the template polynucleotide comprises no more than 10, 20, 30, 40, 50, 100, 200, 300, 400, 500, 600, 700, 800, 900, 1000, 1500, or 2000 base pairs of homology 5′ of the target site or transgene, 3′ of the target site or transgene, or both 5′ and 3′ of the target site or transgene.

a. Transgene Sequences

In some embodiments, the template polynucleotide contains a transgene sequence encoding one or more chains of a recombinant receptor, a chimeric receptor or a portion thereof, such as any recombinant receptor described herein, e.g., in Section III.B, or one or more regions, domains or chains of such recombinant receptor.

In some aspects, the transgene sequences encodes a recombinant receptor that includes an extracellular binding region, transmembrane domain and/or an intracellular region. In some aspects, the transgene sequence can encode all or a portion of the recombinant receptor. In some embodiments, the transgene sequence encodes any recombinant receptor described herein, for example in Section III.B, or a one or more regions, domains or chains thereof. In some aspects, upon integration of the transgene sequence into the endogenous TGFBR2 locus, the resulting modified TGFBR2 locus encodes a recombinant receptor, such as any recombinant receptor described herein, for example, in Section III.B, or a one or more regions, domains or chains thereof. For example, the transgene sequences can include sequence of nucleotides encoding one or more of extracellular regions, transmembrane domains, and intracellular regions that can comprise costimulatory signaling domains, and other domains or portions thereof.

In some aspects, transgene sequences, which are nucleic acid sequences of interest encoding one or more chains of a recombinant receptor or a portion thereof, including coding and/or non-coding sequences and/or partial coding sequences thereof, that are inserted or integrated at the target location in the genome can also be referred to as “transgene,” “transgene sequences,” “exogenous nucleic acids sequences,” “heterologous sequences” or “donor sequences.” In some aspects, the transgene is a nucleic acid sequence that is exogenous or heterologous to an endogenous genomic sequences, such as the endogenous genomic sequences at a specific target locus or target location in the genome, of a T cell, e.g., a human T cell. In some aspects, the transgene is a sequence that is modified or different compared to an endogenous genomic sequence at a target locus or target location of a T cell, e.g., a human T cell. In some aspects, the transgene is a nucleic acid sequence that originates from or is modified compared to nucleic acid sequences from different genes, species and/or origins. In some aspects, the transgene is a sequence that is derived from a sequence from a different locus, e.g., a different genomic region or a different gene, of the same species. In some aspects, exemplary recombinant receptors include any described herein, e.g., in Section III.B.

In some embodiments, nuclease-induced HDR results in an insertion of a transgene (also called “exogenous sequence” or “transgene sequence”) for expression of a transgene for targeted insertion. The template polynucleotide sequence is typically not identical to the genomic sequence where it is placed. A template polynucleotide sequence can contain a non-homologous sequence flanked by two regions of homology to allow for efficient HDR at the location of interest. Additionally, template polynucleotide sequence can comprise a vector molecule containing sequences that are not homologous to the region of interest in cellular chromatin. A template polynucleotide sequence can contain several, discontinuous regions of homology to cellular chromatin. For example, for targeted insertion of sequences not normally present in a region of interest, said sequences can be present in a transgene and flanked by regions of homology to sequence in the region of interest.

In some aspects, the transgene sequence is a sequence that is exogenous or heterologous to an open reading frame of the endogenous genomic TGFBR2 locus a T cell, optionally a human T cell. In some aspects, HDR in the presence of a template polynucleotide containing transgene sequences linked to one or more homology arm(s) that are homologous to sequences near a target site at an endogenous TGFBR2 locus, results in a modified TGFBR2 locus encoding a recombinant receptor or a portion thereof.

In some embodiments, the transgene sequence encodes all or a portion of the various regions, domains or chains of a recombinant receptor, such as a recombinant receptor or various regions, domains or chains described in Section III.B herein.

In some aspects, the transgene is a chimeric sequence, comprising a sequence generated by joining different nucleic acid sequences from different genes, species and/or origins. In some aspects, the transgene contains sequence of nucleotides encoding different regions or domains or portions thereof, from different genes, coding sequences or exons or portions thereof, that are joined or linked. In some aspects the transgene sequences for targeted integration encode a polypeptide or a fragment thereof.

In some embodiments, the transgene sequence can encode a recombinant receptor that is a chimeric receptor, such as a chimeric antigen receptor (CAR), or a portion thereof, such as a domain or region thereof. In some embodiments, the transgene sequence encodes various regions or domains of the recombinant receptor, such as a chimeric antigen receptor (CAR). In some embodiments, the transgene includes a sequence of nucleotides encoding an intracellular region, such as an intracellular region of a CAR. In some embodiments, the transgene also includes a sequence of nucleotides encoding a transmembrane region or a membrane association region, such as a transmembrane region of a CAR. In some embodiments, the transgene also includes a sequence of nucleotides encoding an extracellular region, such as an extracellular region of a CAR. Exemplary chimeric receptors include those described in Sections B.1 and B.3 below.

In some embodiments, the transgene sequence can encode a recombinant receptor, such as a recombinant T cell receptor (TCR), or a portion thereof, such as a domain, region or chain thereof. In some embodiments, the recombinant receptor is a recombinant TCR. In some embodiments, the recombinant receptor, such as a recombinant TCR, comprises two or more separate polypeptide chains, such as TCR alpha (TCRα) and TCR beta (TCRβ) chains. In some aspects, the transgene sequence can encode one or more chains of the recombinant TCR, such as a TCRα or a TCRβ or both. In some aspects, the transgene sequence can encode one or more regions or domains of the recombinant TCR, such as intracellular region, transmembrane region and/or extracellular region of a TCRα or a TCRβ or both. In some aspects, the sequences encoding the TCRα and TCRβ are optionally separated by a multicistronic element, such as a 2A element. Exemplary recombinant TCRs include those described in Section III.B.4 below.

In some aspects, the transgene also contains non-coding, regulatory or control sequences, e.g., sequences required for permitting, modulating and/or regulating expression of the encoded polypeptide or fragment thereof or sequences required to modify a polypeptide. In some embodiments, the transgene does not comprise an intron or lacks one or more introns as compared to a corresponding nucleic acid in the genome if the transgene is derived from a genomic sequence. In some embodiments, the transgene sequence does not comprise an intron. In some of embodiments, the transgene contains sequences encoding a recombinant receptor or a portion thereof, wherein all or a portion of the transgene sequences are codon-optimized, e.g., for expression in human cells.

In some embodiments, the length of the transgene sequences, including coding and non-coding regions, is between or between about 100 to about 10,000 base pairs, such as about 100, 200, 300, 400, 500, 600, 700, 800, 900, 1000, 1500, 2000, 2500, 3000, 3500, 4000, 4500, 5000, 6000, 7000, 8000, 9000 or 10000 base pairs. In some embodiments, the length of the transgene sequence is limited by the maximum length of polynucleotide that can be prepared, synthesized or assembled and/or introduced into the cell or the capacity of the viral vector. In some aspects, the length of the transgene sequence can vary depending on the maximum length of the template polynucleotide and/or the length of the one or more homology arm(s) required.

In some embodiments, genetic disruption-induced HDR results in an insertion or integration of transgene sequences at a target location in the genome. The template polynucleotide sequence is typically not identical to the genomic sequence where it is targeted. A template polynucleotide sequence can contain transgene sequences flanked by two regions of homology to allow for efficient HDR at the location of interest. A template polynucleotide sequence can contain several, discontinuous regions of homology to the genomic DNA. For example, for targeted insertion of sequences not normally present in a region of interest, said sequences can be present in a transgene and flanked by regions of homology to sequence in the region of interest. In some embodiments, the transgene sequences encode a recombinant receptor or a portion thereof, e.g., one or more of an extracellular binding region, transmembrane domain and/or a portion of the intracellular region.

In some aspects, upon targeted integration of the transgene by HDR, the genome of the cell contains a modified TGFBR2 locus, comprising a nucleic acid sequence encoding a recombinant receptor or a portion thereof. In some aspects, the entire recombinant receptor is encoded by the transgene sequences. In some aspects, the transgene sequences also contain sequence of nucleotides encoding other molecules and/or regulatory or control elements, e.g., exogenous promoter, and/or multicistronic elements.

In some embodiments, the transgene sequences also includes a signal sequence encoding a signal peptide, a regulatory or control elements, such as a promoter, and/or one or more multicistronic elements, e.g., a ribosome skip element or an internal ribosome entry site (IRES). In some embodiments, the signal sequence can be placed 5′ of the sequence of nucleotides encoding the recombinant receptor.

Exemplary regions, domains or chains encoded by the transgene sequence are described below, and also can be any region or domain described in Section III.B herein.

(i) Signal Sequence

In some embodiments, the transgene includes a signal sequence encodes a signal peptide. In some aspects, the signal sequence may encode a heterologous or non-native signal peptide, e.g., a signal peptide from a different gene or species or a signal peptide that is different from the signal peptide of the endogenous TGFBR2 locus. In some aspects, exemplary signal sequence includes signal sequence of the GMCSFR alpha chain set forth in SEQ ID NO:24 and encoding the signal peptide set forth in SEQ ID NO:25 or the CD8 alpha signal peptide set forth in SEQ ID NO:26. In the mature form of an expressed recombinant receptor, the signal sequence is cleaved from the remaining portions of the polypeptide. In some aspects, the signal sequence is placed 3′ of a regulatory or control element, e.g., a promoter, such as a heterologous promoter, e.g., a promoter not derived from the TGFBR2 locus. In some aspects, the signal sequence is placed 3′ of one or more multicistronic element(s), e.g., a sequence of nucleotides encoding a ribosome skip sequence and/or an internal ribosome entry site (IRES). In some aspects, the signal sequence can be placed 5′ of the sequence of nucleotides encoding the one or more components of the extracellular region in the transgene. In some embodiments, the signal sequence the most 5′ region present in the transgene, and is linked to one of the homology arms. In some aspects, the signal sequence encoded by the transgene sequence include any signal sequence described herein, for example, in Section III.B.

(ii) Exemplary Chimeric Receptor-Encoding Sequences

In some aspects the transgene sequences for targeted integration include sequences encoding a recombinant receptor that is a chimeric receptor, such as a chimeric antigen receptor (CAR) or a chimeric auto antibody receptor (CAAR). In some aspects, the transgene contains sequence of nucleotides encoding different regions or domains or portions of the recombinant receptor, that can be from different genes, coding sequences or exons or portions thereof, that are joined or linked.

In some embodiments, the encoded recombinant receptor, such as a CAR, contains one or more regions or domains, such as one or more of extracellular region (e.g., containing one or more extracellular binding domain(s) and/or spacers), transmembrane domain and/or intracellular region (e.g., containing primary signaling region or domain and/or one or more costimulatory signaling domains). In some aspects, the encoded CAR further contains other domains, such multimerization domains or linkers.

In some aspects, in the transgene, the sequence of nucleotides encoding the extracellular region is placed between the signal sequence and the nucleotides encoding the spacer. In some aspects, in the transgene, the sequence of nucleotides encoding the extracellular multimerization domain is placed between the sequence of nucleotides encoding the binding domain and the sequence of nucleotides encoding the spacer. In some aspects, the sequence of nucleotides encoding the spacer is placed between the sequence of nucleotides encoding the binding domain and the sequence of nucleotides encoding the transmembrane domain. In some embodiments, the transgene includes, in 5′ to 3′ order, a sequence of nucleotides encoding an extracellular region, a sequence of nucleotides a transmembrane domain (or a membrane association domain) and a sequence of nucleotides an intracellular region.

In some embodiments, the encoded recombinant receptor is a CAR, and the transgene that encodes an extracellular region can include, in 5′ to 3′ order, a sequence of nucleotides encoding an extracellular binding domain and a sequence of nucleotides encoding a spacer. In some embodiments, the transgene also includes a sequence of nucleotides encoding one or more extracellular multimerization domain(s), which can be placed 5′ or 3′ of any of the sequence of nucleotides encoding binding domains and/or spacers, and/or 5′ of the sequence of nucleotides encoding a transmembrane domain. In some aspects, the transgene sequence also includes a signal sequence, typically placed 5′ of the sequence of nucleotides encoding the extracellular region.

In some aspects, in the transgene, the sequence of nucleotides encoding the binding domain is placed between the signal sequence and the nucleotides encoding the spacer. In some aspects, in the transgene, the sequence of nucleotides encoding the extracellular multimerization domain is placed between the sequence of nucleotides encoding the binding domain and the sequence of nucleotides encoding the spacer. In some aspects, the sequence of nucleotides encoding the spacer is placed between the sequence of nucleotides encoding the binding domain and the sequence of nucleotides encoding the transmembrane domain.

In some embodiments, the transgene contains a sequence of nucleotides encoding an intracellular region, which can include a sequence of nucleotides encoding one or more costimulatory signaling domain(s) and/or a primary signaling domain or region.

In some embodiments, the transgene also comprises one or more multicistronic element(s), e.g., a ribosome skip sequence and/or an internal ribosome entry site (IRES). In some aspects, the transgene also includes regulatory or control elements, such as a promoter, typically at the most 5′ portion of the transgene sequence, e.g., 5′ of the signal sequence. In some aspects, sequence of nucleotides encoding one or more additional molecule(s) or additional domains or regions can be included in the transgene portion of the polynucleotide. In some aspects, the sequence of nucleotides encoding one or more additional molecule(s) or additional domains or regions can be placed 5′ of the sequence of nucleotides encoding one or more region(s) or domain(s) or chain(s) of the CAR. In some aspects, the sequence of nucleotides encoding the one or more additional molecule(s) or additional domains, regions or chains is upstream of the sequence of nucleotides encoding one or more regions of the CAR.

Exemplary domains or regions of the chimeric receptor encoded by the transgene sequences are described below, and also can include any region or domain of exemplary chimeric receptors described in Sections III.B.1 and III.B.3 below.

(a) Binding Domain

In some embodiments, the transgene encodes a portion of a recombinant receptor, such as a CAR with specificity for a particular antigen (or ligand), such as an antigen expressed on the surface of a particular cell type. In some embodiments, the antigen is selectively expressed or overexpressed on cells of the disease or condition, e.g., the tumor or pathogenic cells, as compared to normal or non-targeted cells or tissues, e.g., in healthy cells or tissues.

In some aspects, the transgene encodes an extracellular region of a recombinant receptor. In some embodiments, the transgene sequences encode extracellular binding domain, such as a binding domain that specifically binds an antigen or a ligand.

In some embodiments, the binding domain is or comprises a polypeptide, a ligand, a receptor, a ligand-binding domain, a receptor-binding domain, an antigen, an epitope, an antibody, an antigen-binding domain, an epitope-binding domain, an antibody-binding domain, a tag-binding domain or a fragment of any of the foregoing. In other embodiments, the antigen is expressed on normal cells and/or is expressed on the engineered cells. In some aspects, the antigen is recognized by a binding domain, such as a ligand binding domain or an antigen binding domain. In some aspects, the transgene encodes an extracellular region containing one or more binding domain(s). In some embodiments, exemplary binding domain encoded by the transgene include antibodies and antigen-binding fragments thereof, including scFv or sdAb. In some embodiments, an antigen-binding fragment comprises antibody variable regions joined by a flexible linker.

In some embodiments, the binding domain is or comprises a single chain variable fragment (scFv). In some embodiments, the binding domain is or comprises a single domain antibody (sdAb). In some embodiments, the binding domain is capable of binding to a target antigen that is associated with, specific to, and/or expressed on a cell or tissue of a disease, disorder or condition. In some embodiments, the disease, disorder or condition is an infectious disease or disorder, an autoimmune disease, an inflammatory disease, or a tumor or a cancer. In some embodiments, the target antigen is a tumor antigen.

Exemplary antigens and antigen- or ligand-binding domains encoded by the transgene sequences include those described in Section III.B.1 herein. In some aspects, the encoded recombinant receptor contains a binding domain that is or comprises a TCR-like antibody or a fragment thereof, such as an scFv that specifically recognizes an intracellular antigen, such as a tumor-associated antigen, presented on the cell surface as a major histocompatibility complex (MHC)-peptide complex. In some aspects, the transgene sequences can encode a binding domain that is a TCR-like antibody or fragment thereof. Thus, the encoded recombinant receptor is a TCR-like CAR, such as any described herein in Section III.B. In some embodiments, the binding domain is a multi-specific, such as a bi-specific, binding domain. In some embodiments, the encoded recombinant receptor contains a binding domain that is an antigen that binds to an autoantibody. In some embodiments, the recombinant receptor is a chimeric auto antibody receptor (CAAR), such as any described herein in Section III.B.3.

In some aspects, sequence of nucleotides encoding the one or more binding domain(s) can be placed 3′ of a signal sequence, if present, in the transgene. In some aspects, sequence of nucleotides encoding the one or more binding domain(s) can be placed 3′ of the sequence of nucleotides encoding one or more regulatory or control element(s), in the transgene. In some aspects, sequence of nucleotides encoding the one or more binding domain(s) can be placed 5′ of the sequence of nucleotides encoding the spacer, if present, in the transgene. In some aspects, sequence of nucleotides encoding the one or more binding domain(s) can be placed 5′ of the sequence of nucleotides encoding transmembrane domain, in the transgene.

(b) Spacer and Transmembrane Domain

In some embodiments, the encoded recombinant receptor is a CAR, and the transgene includes sequences encoding a spacer and/or sequences encoding a transmembrane domain or portion thereof. In some embodiments, the extracellular region of the encoded recombinant receptor comprises a spacer, optionally wherein the spacer is operably linked between the binding domain and the transmembrane domain. In some aspects, the spacer and/or transmembrane domain can link the extracellular portion containing the ligand- (e.g., antigen-) binding domain and other regions or domains of the recombinant receptor, such as the intracellular region (e.g., containing one or more costimulatory signaling domain(s), intracellular multimerization domain and/or a primary signaling domain or region).

In some embodiments, the transgene further includes sequence of nucleotides encoding a spacer and/or a hinge region that separates the antigen-binding domain and transmembrane domain. In some aspects, the spacer may be or include at least a portion of an immunoglobulin constant region or variant or modified version thereof, such as a hinge region, e.g., an IgG4 hinge region, and/or a C_{H}l/C_{L} and/or Fc region. In some embodiments, the constant region or portion is of a human IgG, such as IgG4 or IgG1. In some aspects, the portion of the constant region serves as a spacer region between a binding domain, e.g., scFv, and a transmembrane domain. Exemplary spacers that can be encoded by the transgene include IgG4 hinge alone, IgG4 hinge linked to C_{H}2 and C_{H}3 domains, or IgG4 hinge linked to the C_{H}3 domain, and those described in Hudecek et al. (2013) Clin. Cancer Res., 19:3153, Hudecek et al. (2015) Cancer Immunol Res. 3(2): 125-135 or International Pat. App. Pub. No. WO2014031687, or any described in Section III.B.1 herein.

In some aspects, the sequence of nucleotides encoding the spacer can be placed 3′ of the sequence of nucleotides encoding the one or more binding domains, in the transgene. In some aspects, the sequence of nucleotides encoding the spacer can be placed 5′ of the sequence of nucleotides encoding the transmembrane domain, in the transgene. In some embodiments, the sequence of nucleotides encoding the spacer is placed between the sequence of nucleotides encoding one or more binding domains and the sequence of nucleotides encoding the transmembrane domain.

In some embodiments, the transgene encodes a transmembrane domain, which can link the extracellular region, e.g., containing one or more binding domains and/or spacers, with the intracellular region, e.g., containing one or more costimulatory signaling domain(s), intracellular multimerization domain and/or a primary signaling domain or region. In some embodiments, the transgene comprises a sequence of nucleotides encoding a transmembrane domain, optionally wherein the transmembrane domain is human or comprises a sequence from a human protein. In some embodiments, the transmembrane domain is or comprises a transmembrane domain derived from CD4, CD28, or CD8, optionally derived from human CD4, human CD28 or human CD8. In some embodiments, the transmembrane domain is or comprises a transmembrane domain derived from a CD28, optionally derived from human CD28.

In some embodiments, the sequence of nucleotides encoding transmembrane domain is fused to the sequence of nucleotides encoding the extracellular region. In some embodiments, the sequence of nucleotides encoding transmembrane domain is fused to the sequence of nucleotides encoding the intracellular region. In some aspects, sequence of nucleotides encoding the transmembrane domain can be placed 3′ of the sequence of nucleotides encoding the one or more binding domains and/or the spacer in the transgene. In some aspects, the sequence of nucleotides encoding the transmembrane domain can be placed 5′ of the sequence of nucleotides encoding the intracellular region, e.g., containing one or more costimulatory signaling domain(s), intracellular multimerization domain and/or a primary signaling domain or region, in the transgene. In some aspects, the transmembrane domain encoded by the transgene sequence include any transmembrane domain described herein, for example, in Section III.B.1.

In some embodiments, in cases where the encoded recombinant receptor comprises an intracellular region comprising a primary signaling domain or region but does not comprise a transmembrane domain and/or an extracellular region, the transgene can include a sequence of nucleotides encoding a membrane association domain, such as any described herein, e.g., in Section III.B.

(c) Intracellular Region

In some embodiments, the transgene includes a sequence of nucleotides encoding an intracellular region. In some embodiments, the transgene encodes a CAR, and in some aspects, the intracellular region comprises one or more secondary or co-stimulatory signaling region. In some aspects, the sequence of nucleotides encoding the transmembrane domain can be placed 3′ of the sequence of nucleotides encoding the one or more binding domains and/or the spacer in the transgene, in the transgene. In some aspects, the sequence of nucleotides encoding the one or more costimulatory signaling domain can be placed 5′ of the sequence of nucleotides encoding a primary signaling domain or region. In some aspects, the sequence of nucleotides encoding the one or more costimulatory signaling domain can be placed 3′ of the sequence of nucleotides encoding a primary signaling domain or region. In some aspects, the sequence of nucleotides encoding intracellular region is the most 3′ region in the transgene, which is then linked to one of the homology arm sequences, e.g., the 3′ homology arm sequence. In some aspects, the sequence of nucleotides encoding the one or more costimulatory signaling domain can be placed 3′ of the sequence of nucleotides encoding the transmembrane domain, in the transgene. In some aspects, the costimulatory signaling region or the primary signaling domain or region encoded by the transgene sequence include any costimulatory signaling region or any primary signaling domain or region described herein, for example, in Section III.B.1.

(1) Costimulatory Signaling Domain

In some embodiments, the transgene comprises a sequence of nucleotides encoding a portion of the intracellular region, which can include one or more costimulatory signaling domain(s). In some embodiments, the one or more costimulatory signaling domain comprises an intracellular signaling domain of a T cell costimulatory molecule or a signaling portion thereof, optionally wherein the T cell costimulatory molecule or a signaling portion thereof is human.

In some embodiments, the one or more costimulatory signaling domain comprises an intracellular signaling domain of a T cell costimulatory molecule or a signaling portion thereof. In some embodiments, the T cell costimulatory molecule or a signaling portion thereof is human. In some embodiments, exemplary costimulatory signaling domain encoded by the transgene include signaling regions or domains from one or more costimulatory receptor such as CD28, CD137 (4-1BB), OX40 (CD134), CD27, DAP10, DAP12, NKG2D, ICOS and/or other costimulatory receptors, such as any described herein in Section III.B herein. In some embodiments, the one or more costimulatory signaling domain comprises an intracellular signaling domain of a CD28, a 4-1BB or an ICOS or a signaling portion thereof. In some embodiments, the one or more costimulatory signaling domain comprises a signaling domain of human CD28, human 4-1BB, human ICOS or a signaling portion thereof. In some embodiments, the one or more costimulatory signaling domain comprises an intracellular signaling domain of human 4-1BB.

(2) Primary Signaling Region or Domain

In some embodiments, the transgene sequence encoding a recombinant receptor, e.g., CAR, includes a sequence of nucleotides encoding a primary signaling region or domain, such as the cytoplasmic domain of CD3zeta (CD3ζ). In some embodiments, the primary signaling region is or comprises a signaling domain that is capable of stimulating and/or inducing a primary activation signal in a T cell, a signaling domain of a T cell receptor (TCR) component (e.g. an intracellular signaling domain or region of a CD3-zeta (CD3ζ) chain or a functional variant or signaling portion thereof) and/or a signaling domain comprising an immunoreceptor tyrosine-based activation motif (ITAM). In some embodiments, the encoded recombinant receptor is any describe herein, for example, in Section III.B.

In some aspects, the transgene includes a sequence of nucleotides encoding a primary cytoplasmic signaling region that regulates primary stimulation and/or activation of the TCR complex. Primary cytoplasmic signaling region(s) that act in a stimulatory manner may contain signaling motifs which are known as immunoreceptor tyrosine-based activation motifs or ITAMs. Examples of ITAM containing primary cytoplasmic signaling region(s) include those derived from TCR or CD3 zeta (CD3ζ), Fc receptor (FcR) gamma or FcR beta. In some embodiments, cytoplasmic signaling regions or domains in the CAR contain(s) a cytoplasmic signaling domain, portion thereof, or sequence derived from CD3 zeta. In some embodiments, the intracellular (or cytoplasmic) signaling region comprises a human CD3 chain, optionally a CD3 zeta stimulatory signaling domain or functional variant thereof, such as an 112 AA cytoplasmic domain of isoform 3 of human CD3ζ (Accession No.: P20963.2) or a CD3 zeta signaling domain as described in U.S. Pat. Nos. 7,446,190 or 8,911,993. In some embodiments, the intracellular signaling region comprises the sequence of amino acids set forth in SEQ ID NO: 13, 14 or 15 or a sequence of amino acids that exhibits at least or at least about 85%, 86%, 87%, 88%, 89%, 90%, 91%, 92%, 93%, 94%, 95%, 96%, 97%, 98%, 99% or more sequence identity to SEQ ID NO: 13, 14 or 15.

In some aspects, the primary signaling domain or region encoded by the transgene sequence include any primary signaling domain or region described herein, for example, in Section III.B.1.

(d) Additional Domains, e.g., Multimerization Domains

In some embodiments, the transgene also includes a sequence of nucleotides encoding one or more multimerization domain(s), e.g., a dimerization domain. In some aspects, the encoded multimerization domain can be extracellular or intracellular. In some embodiments, the encoded multimerization domain is extracellular. In some embodiments, the encoded multimerization domain is intracellular. In some embodiments, the portion of the intracellular region encoded by the transgene sequences comprises a multimerization domain, optionally a dimerization domain. In some embodiments, the transgene comprises a sequence of nucleotides encoding an extracellular region. In some embodiments, the extracellular region comprises a multimerization domain, optionally a dimerization domain. In some embodiments, the multimerization domain is capable of dimerization upon binding to an inducer.

In some aspects, the recombinant receptor is a multi-chain recombinant receptor, such as a multi-chain CAR. In some embodiments, one or more chains of the multi-chain recombinant receptor or a portion thereof is encoded by the transgene sequence. In some embodiments, one or more chains of the multi-chain recombinant receptor can together form a functional or active recombinant receptor, by virtue of multimerization of the multimerization domain included in each chain of the recombinant receptor.

In some aspects, the sequence of nucleotides encoding a multimerization domain is 5′ or 3′ of other domains. For example, in some embodiments, the encoded multimerization domain is extracellular, and the sequence encoding the multimerization domain is 5′ of the sequence encoding the spacer. In some embodiments, the encoded multimerization domain is intracellular, and the sequence encoding the multimerization domain is 5′ of the sequence encoding the primary signaling region or domain. In some embodiments, the multimerization domain is intracellular, and the sequence encoding the multimerization domain is 5′ or 3′ of the sequence encoding one or more costimulatory signaling domain(s). In some embodiments, the encoded multimerization domain can multimerize (e.g., dimerize), upon binding of an inducer. Exemplary encoded multimerization domain includes any multimerization domain described herein, e.g., in Section III.B herein.

(iii) Exemplary T Cell Receptor (TCR)-Encoding Sequences

In some embodiments, the recombinant receptor encoded by the transgene sequences is a recombinant T cell receptor (TCR). In some aspects, the transgene sequence can encode all or a portion of the recombinant TCR. In some embodiments, the transgene sequence comprises a sequence of nucleotides encoding one or more chains, regions or domains of a recombinant TCR. Exemplary recombinant TCR encoded by the transgene sequences are described below, and also can include any chains, region or domain of exemplary recombinant TCRs described in Sections B.4 below.

In some embodiments, the TCR, comprises two or more separate polypeptide chains such as TCR alpha (TCRα) and TCR beta (TCRβ) chains. In some aspects, the transgene sequence can encode one or more chains of the recombinant TCR, such as a TCRα or a TCRβ or both. In some aspects, the transgene sequence can encode both TCRα and TCRβ chains. In some aspects, the sequences encoding the TCRα and TCRβ are optionally separated by a multicistronic element, such as a 2A element.

In certain embodiments, the transgene includes nucleic acid sequence encoding recombinant receptor is a recombinant TCR or an antigen-binding fragment thereof. In some aspects, the transgene sequence can encode a chain if the recombinant TCR, containing a variable domain and a constant domain. In some aspects, the transgene sequence encodes a chain of a recombinant TCR that contains one or more variable domains and one or more constant domains. In some of embodiments, the transgene contains a sequence encoding a TCRα and a TCRβ chain.

In some embodiments, the encoded TCRα chain and TCRβ chain are separated by a linker region. In some embodiments, a linker sequence is included that links the TCRα and TCRβ chains to form the single polypeptide strand. In some embodiments, the linker is of sufficient length to span the distance between the C terminus of the α chain and the N terminus of the β chain, or vice versa, while also ensuring that the linker length is not so long so that it blocks or reduces bonding to a target peptide-MHC complex. In some embodiments, the linker may be any linker capable of forming a single polypeptide strand, while retaining TCR binding specificity. In some embodiments, the linker can contain from or from about 10 to 45 amino acids, such as 10 to 30 amino acids or 26 to 41 amino acids residues, for example 29, 30, 31 or 32 amino acids. In some embodiments, the linker has the formula -PGGG-(SGGGG)n-P-, wherein n is 5 or 6 and P is proline, G is glycine and S is serine (SEQ ID NO: 22). In some embodiments, the linker has the sequence GSADDAKKDAAKKDGKS (SEQ ID NO: 23). In some embodiments, the linker between the TCRα chain or portion thereof and the TCRβ chain or portion thereof that is recognized by and/or is capable of being cleaved by a protease. In certain embodiments, the linker between the nucleic acid sequence encoding a TCRα chain or portion thereof and the nucleic acid sequence encoding a TCRβ chain or portion thereof contains a multicistronic element.

In some embodiments, the transgene is or include a sequence of nucleotides that is or includes the structure [TCRβ chain]-[linker or multicistronic element]-[TCRα chain]. In particular embodiments, the transgene is or include a sequence of nucleotides that is or includes the structure [TCRα chain]-[linker or multicistronic element]-[TCRβ chain]. In some aspects, the multicistronic element includes a ribosome skipping element/self-cleavage element (e.g., a 2A element or an internal ribosome entry site (IRES), such as any described herein.

(iv) Additional Molecules, e.g., Markers

In some embodiments, the transgene also includes a sequence of nucleotides encoding one or more additional molecules, such as an antibody, an antigen, an additional chimeric or additional polypeptide chains of a multi-chain recombinant receptor (e.g., multi-chain CAR, chimeric co-stimulatory receptor, inhibitory receptor, regulatable chimeric antigen receptor or other components of multi-chain recombinant receptor systems described herein, for example, in Section III.B.2 or a recombinant T cell receptor (TCR) described in Section III.B.3), a transduction marker or a surrogate marker (e.g., truncated cell surface marker), an enzyme, an factors, a transcription factor, an inhibitory peptide, a growth factor, a nuclear receptor, a hormone, a lymphokine, a cytokine, a chemokine, a soluble receptor, a soluble cytokine receptor, a soluble chemokine receptor, a reporter, functional fragments or functional variants of any of the foregoing and combinations of the foregoing. In some aspects, such sequence of nucleotides encoding one or more additional molecules can be placed 5′ of the sequence of nucleotides encoding regions or domains of the recombinant receptor. In some aspects, the sequences encoding one or more other molecules and the sequence of nucleotides encoding regions or domains of the recombinant receptor are separated by regulatory sequences, such as a 2A ribosome skipping element and/or promoter sequences.

In some embodiments, the transgene also includes a sequence of nucleotides encoding one or more additional molecules. In some aspects, one or more additional molecules include one or more marker(s). In some embodiments, the one or more marker(s) includes a transduction marker, a surrogate marker and/or a selection marker. In some embodiments, the transgene also includes nucleic acid sequences that can improve the efficacy of therapy, such as by promoting viability and/or function of transferred cells; nucleic acid sequences to provide a genetic marker for selection and/or evaluation of the cells, such as to assess in vivo survival or localization; nucleic acid sequences to improve safety, for example, by making the cell susceptible to negative selection in vivo as described by Lupton S. D. et al., Mol. and Cell Biol., 11:6 (1991); and Riddell et al., Human Gene Therapy 3:319-338 (1992); see also WO 1992008796 and WO 1994028143 describing the use of bifunctional selectable fusion genes derived from fusing a dominant positive selectable marker with a negative selectable marker, and U.S. Pat. No. 6,040,177. In some aspects, the markers include any markers described herein, for example, in this section or Sections II or III.B, or any additional molecules and/or receptor polypeptides described herein, for example, in Section III.B.2. In some embodiments, the additional molecule is a surrogate marker, optionally a truncated receptor, optionally wherein the truncated receptor lacks an intracellular signaling domain and/or is not capable of mediating intracellular signaling when bound by its ligand.

In some embodiments, the marker is a transduction marker or a surrogate marker. A transduction marker or a surrogate marker can be used to detect cells that have been introduced with the polynucleotide, e.g., a polynucleotide encoding a recombinant receptor. In some embodiments, the transduction marker can indicate or confirm modification of a cell. In some embodiments, the surrogate marker is a protein that is made to be co-expressed on the cell surface with the recombinant receptor, e.g. TCR or CAR. In particular embodiments, such a surrogate marker is a surface protein that has been modified to have little or no activity. In certain embodiments, the surrogate marker is encoded on the same polynucleotide that encodes the recombinant receptor. In some embodiments, the nucleic acid sequence encoding the recombinant receptor is operably linked to a nucleic acid sequence encoding a marker, optionally separated by an internal ribosome entry site (IRES), or a nucleic acid encoding a self-cleaving peptide or a peptide that causes ribosome skipping, such as a 2A sequence, such as a T2A, a P2A, an E2A or an F2A. Extrinsic marker genes may in some cases be utilized in connection with engineered cell to permit detection or selection of cells and, in some cases, also to promote cell elimination and/or cell suicide.

Exemplary surrogate markers can include truncated forms of cell surface polypeptides, such as truncated forms that are non-functional and to not transduce or are not capable of transducing a signal or a signal ordinarily transduced by the full-length form of the cell surface polypeptide, and/or do not or are not capable of internalizing. Exemplary truncated cell surface polypeptides including truncated forms of growth factors or other receptors such as a truncated human epidermal growth factor receptor 2 (tHER2), a truncated epidermal growth factor receptor (tEGFR, exemplary tEGFR sequence set forth in SEQ ID NO:7 or 16) or a prostate-specific membrane antigen (PSMA) or modified form thereof. tEGFR may contain an epitope recognized by the antibody cetuximab (Erbitux®) or other therapeutic anti-EGFR antibody or binding molecule, which can be used to identify or select cells that have been engineered with the tEGFR construct and an encoded exogenous protein, and/or to eliminate or separate cells expressing the encoded exogenous protein. See U.S. Pat. No. 8,802,374 and Liu et al., Nature Biotech. 2016 April; 34(4): 430-434). In some aspects, the marker, e.g. surrogate marker, includes all or part (e.g., truncated form) of CD34, a NGFR, a CD19 or a truncated CD19, e.g., a truncated non-human CD19, or epidermal growth factor receptor (e.g., tEGFR).

In some embodiments, the marker is or comprises a detectable protein, such as a fluorescent protein, such as green fluorescent protein (GFP), enhanced green fluorescent protein (EGFP), such as super-fold GFP (sfGFP), red fluorescent protein (RFP), such as tdTomato, mCherry, mStrawberry, AsRed2, DsRed or DsRed2, cyan fluorescent protein (CFP), blue green fluorescent protein (BFP), enhanced blue fluorescent protein (EBFP), and yellow fluorescent protein (YFP), and variants thereof, including species variants, monomeric variants, codon-optimized, stabilized and/or enhanced variants of the fluorescent proteins. In some embodiments, the marker is or comprises an enzyme, such as a luciferase, the lacZ gene from E. coli, alkaline phosphatase, secreted embryonic alkaline phosphatase (SEAP), chloramphenicol acetyl transferase (CAT). Exemplary light-emitting reporter genes include luciferase (luc), β-galactosidase, chloramphenicol acetyltransferase (CAT), β-glucuronidase (GUS) or variants thereof. In some aspects, expression of the enzyme can be detected by addition of a substrate that can be detected upon the expression and functional activity of the enzyme.

In some embodiments, the marker is a selection marker. In some embodiments, the selection marker is or comprises a polypeptide that confers resistance to exogenous agents or drugs. In some embodiments, the selection marker is an antibiotic resistance gene. In some embodiments, the selection marker is an antibiotic resistance gene confers antibiotic resistance to a mammalian cell. In some embodiments, the selection marker is or comprises a Puromycin resistance gene, a Hygromycin resistance gene, a Blasticidin resistance gene, a Neomycin resistance gene, a Geneticin resistance gene or a Zeocin resistance gene or a modified form thereof.

In some embodiments, the molecule is a non-self molecule, e.g., non-self protein, i.e., one that is not recognized as “self” by the immune system of the host into which the cells will be adoptively transferred.

In some embodiments, the marker serves no therapeutic function and/or produces no effect other than to be used as a marker for genetic engineering, e.g., for selecting cells successfully engineered. In other embodiments, the marker may be a therapeutic molecule or molecule otherwise exerting some desired effect, such as a ligand for a cell to be encountered in vivo, such as a costimulatory or immune checkpoint molecule to enhance and/or dampen responses of the cells upon adoptive transfer and encounter with ligand.

In some embodiments, the transgene includes sequences encoding one or more additional molecule that is an immunomodulatory agent. In some embodiments, the immunomodulatory molecule is selected from an immune checkpoint modulator, an immune checkpoint inhibitor, a cytokine or a chemokine. In some embodiments, the immunomodulatory agent is an immune checkpoint inhibitor capable of inhibiting or blocking a function of an immune checkpoint molecule or a signaling pathway involving an immune checkpoint molecule. In some embodiments, the immune checkpoint molecule is selected from among PD-1, PD-L1, PD-L2, CTLA-4, LAG-3, TIM3, VISTA, an adenosine receptor or extracellular adenosine, optionally an adenosine 2A Receptor (A2AR) or adenosine 2B receptor (A2BR), or adenosine or a pathway involving any of the foregoing. Other exemplary additional molecules include epitope tags, detectable molecules such as fluorescent or luminescent proteins, or molecules that mediate enhanced cell growth and/or gene amplification (e.g., dihydrofolate reductase). Epitope tags include, for example, one or more copies of FLAG, His, myc, Tap, HA or any detectable amino acid sequence. In some embodiments, additional molecules can include non-coding sequences, inhibitory nucleic acid sequences, such as antisense RNAs, RNAi, shRNAs and micro RNAs (miRNAs), or nuclease recognition sequences.

In some aspects, the additional molecule can include any additional receptor polypeptides described herein, such as any additional polypeptide chain of the multi-chain recombinant receptor, e.g., as described in Section III.B.2.

(v) Multicistronic Elements and Regulatory or Control Elements

In some embodiments, the transgene (e.g., exogenous nucleic acid sequences) also contains one or more heterologous or exogenous regulatory or control elements, e.g., cis-regulatory elements, that are not, or are different from the regulatory or control elements of the endogenous TGFBR2 locus. In some aspects, the heterologous regulatory or control elements include such as a promoter, an enhancer, an intron, an insulator, a polyadenylation signal, a transcription termination sequence, a Kozak consensus sequence, a multicistronic element (e.g., internal ribosome entry sites (IRES), a 2A sequence), sequences corresponding to untranslated regions (UTR) of a messenger RNA (mRNA), and splice acceptor or donor sequences, such as those that are not, or are different from the regulatory or control element at the TGFBR2 locus. In some embodiments, the heterologous regulatory or control elements include a promoter, an enhancer, an intron, a polyadenylation signal, a Kozak consensus sequence, a splice acceptor sequence and/or a splice donor sequence. In some embodiments, the transgene comprises a promoter that is heterologous and/or not typically present at or near the target site. In some aspects, the regulatory or control element includes elements required to regulate or control the expression of the recombinant receptor, when integrated at the TGFBR2 locus. In some embodiments, the transgene sequences include sequences corresponding to 5′ and/or 3′ untranslated regions (UTRs) of a heterologous gene or locus. In some aspects, the transgene sequence can include any regulatory or control elements described herein, including those described in this section and Section II.

The transgene, including the transgene encoding the one or more chains of a recombinant receptor or a portion thereof, can be inserted so that its expression is driven by the endogenous promoter at the integration site, namely the promoter that drives expression of the endogenous TGFBR2 gene. In some embodiments in which the polypeptide encoding sequences are promoterless, expression of the integrated transgene is then ensured by transcription driven by an endogenous promoter or other control element in the region of interest. For example, the transgene encoding a portion of the recombinant receptor can be inserted without a promoter, but in-frame with the coding sequence of the endogenous TGFBR2 locus, such that expression of the integrated transgene is controlled by the transcription of the endogenous promoter and/or other regulatory elements at the integration site. In some embodiments, a multicistronic element such as a ribosome skipping element/self-cleavage element (e.g., a 2A element or an internal ribosome entry site (IRES)), is placed upstream of the transgene encoding a portion of the recombinant receptor, such that the multicistronic element is placed in-frame with one or more exons of the endogenous open reading frame at the TGFBR2 locus, such that the expression of the transgene encoding the recombinant receptor is operably linked to the endogenous TGFBR2 promoter. In some embodiments, the transgene sequence does not comprise a sequence encoding a 3′ UTR. In some embodiments, upon integration of the transgene into the endogenous TGFBR2 locus, the transgene is integrated upstream of the 3′ UTR of the endogenous TGFBR2 locus, such that the message encoding the recombinant receptor contains a 3′ UTR of the endogenous TGFBR2 locus, e.g., from the open reading frame or partial sequence thereof of the endogenous TGFBR2 locus. In some embodiments, the open reading frame or a partial sequence thereof encoding the remaining portion of the recombinant receptor comprises a 3′ UTR of the endogenous TGFBR2 locus.

In some embodiments, a “tandem” cassette is integrated into the selected site. In some embodiments, one or more of the “tandem” cassettes encode one or more polypeptide or factors, each independently controlled by a regulatory element or all controlled as a multi-cistronic expression system. In some embodiments, such as those where the polynucleotide contains a first and second nucleic acid sequence, the coding sequences encoding each of the different polypeptide chains can be operatively linked to a promoter, which can be the same or different. In some embodiments, the nucleic acid molecule can contain a promoter that drives the expression of two or more different polypeptide chains. In some embodiments, such nucleic acid molecules can be multicistronic (bicistronic or tricistronic, see e.g., U.S. Pat. No. 6,060,273). In some embodiments, transcription units can be engineered as a bicistronic unit containing an IRES (internal ribosome entry site), which allows coexpression of gene products by a message from a single promoter. Alternatively, in some cases, a single promoter may direct expression of an RNA that contains, in a single open reading frame (ORF), two or three polypeptides separated from one another by sequences encoding a self-cleavage peptide (e.g., 2A sequences) or a protease recognition site (e.g., furin), as described herein. The ORF thus encodes a single polypeptide, which, either during (in the case of 2A) or after translation, is processed into the individual proteins. In some embodiments, the “tandem cassette” includes the first component of the cassette comprising a promoterless sequence, followed by a transcription termination sequence, and a second sequence, encoding an autonomous expression cassette or a multi-cistronic expression sequence. In some embodiments, the tandem cassette encodes two or more different polypeptides or factors, e.g., two or more chains or domains of a recombinant receptor. In some embodiments, nucleic acid sequences encoding two or more chains or domains of the recombinant receptor are introduced as tandem expression cassettes or bi- or multi-cistronic cassettes, into one target DNA integration site.

In some cases, the multicistronic element, such as a T2A, can cause the ribosome to skip (ribosome skipping) synthesis of a peptide bond at the C-terminus of a 2A element, leading to separation between the end of the 2A sequence and the next peptide downstream (see, for example, de Felipe, Genetic Vaccines and Ther. 2:13 (2004) and de Felipe et al. Traffic 5:616-626 (2004); also referred to as a self-cleavage element). This allows the inserted transgene to be controlled by the transcription of the endogenous promoter at the integration site such as a TGFBR2 promoter. Exemplary multicistronic element include 2A sequences from the foot-and-mouth disease virus (F2A, e.g., SEQ ID NO: 21), equine rhinitis A virus (E2A, e.g., SEQ ID NO: 20), Thosea asigna virus (T2A, e.g., SEQ ID NO: 6 or 17), and porcine teschovirus-1 (P2A, e.g., SEQ ID NO: 18 or 19) as described in U.S. Patent Pub. No. 20070116690. In some embodiments, the template polynucleotide includes a P2A ribosome skipping element (sequence set forth in SEQ ID NO: 18 or 19) upstream of the transgene, e.g., nucleic acids encoding the recombinant receptor or portion thereof.

In some embodiments, the transgene encoding the one or more chains of a recombinant receptor or portion thereof and/or the sequences encoding an additional molecule independently comprises one or more multicistronic element(s). In some embodiments, the one or more multicistronic element(s) are upstream of the nucleic acid sequence encoding the recombinant receptor portion thereof and/or the sequences encoding an additional molecule. In some embodiments, the multicistronic element(s) is positioned between the nucleic acid sequence encoding the recombinant receptor portion thereof and/or the sequences encoding an additional molecule. In some embodiments, the multicistronic element(s) is positioned between the nucleic acid sequence encoding portions or chains of the recombinant receptor.

In some embodiments, the heterologous regulatory or control element comprises a heterologous promoter. In some embodiments, the heterologous promoter is selected from among a constitutive promoter, an inducible promoter, a repressible promoter, and/or a tissue-specific promoter. In some embodiments, regulatory or control element is a promoter and/or enhancer, for example a constitutive promoter or an inducible or tissue-specific promoter. In some embodiments, the promoter is selected from among an RNA pol I, pol II or pol III promoter. In some embodiments, the promoter is recognized by RNA polymerase II (e.g., a CMV, SV40 early region or adenovirus major late promoter). In some embodiments, the promoter is recognized by RNA polymerase III (e.g., a U6 or H1 promoter). In some embodiments, the promoter is or comprises a constitutive promoter. Exemplary constitutive promoters include, e.g., simian virus 40 early promoter (SV40), cytomegalovirus immediate-early promoter (CMV), human Ubiquitin C promoter (UBC), human elongation factor 1α promoter (EF1α), mouse phosphoglycerate kinase 1 promoter (PGK), and chicken R-Actin promoter coupled with CMV early enhancer (CAGG). In some embodiments, the heterologous promoter is or comprises a human elongation factor 1 alpha (EF1α) promoter or an MND promoter or a variant thereof.

In some embodiments, the promoter is a regulated promoter (e.g., inducible promoter). In some embodiments, the promoter is an inducible promoter or a repressible promoter. In some embodiments, the promoter comprises a Lac operator sequence, a tetracycline operator sequence, a galactose operator sequence, a doxycycline operator sequence, or a transforming growth factor beta (TGFβ) responsive element or is an analog thereof or is capable of being bound by or recognized by a Lac repressor or a tetracycline repressor or a TGFβ responsive transcription factor, or an analog thereof. Exemplary TGFβ responsive elements include those described in, for example, Mostert et al., (2001) Eur. J. Biochem 268:6176-6181; Denissova et al., (2000) Proc Natl Acad Sci USA. 2000 Jun. 6; 97(12):6397-402; Riccio et al., (1992) Mol. Cel. Boil. 12(4):1846-1855; and Boon et al., (2007) Arteriosclerosis, Thrombosis, and Vascular Biology 27:532-539. In some embodiments, the promoter is a tissue-specific promoter. In some instances, the promoter is only expressed in a specific cell type (e.g., a T cell or B cell or NK cell specific promoter).

In some embodiments, the promoter is or comprises a constitutive promoter. Exemplary constitutive promoters include, e.g., simian virus 40 early promoter (SV40), cytomegalovirus immediate-early promoter (CMV), human Ubiquitin C promoter (UBC), human elongation factor 1α promoter (EF1α), mouse phosphoglycerate kinase 1 promoter (PGK), and chicken R-Actin promoter coupled with CMV early enhancer (CAGG). In some embodiments, the constitutive promoter is a synthetic or modified promoter. In some embodiments, the promoter is or comprises an MND promoter, a synthetic promoter that contains the U3 region of a modified MoMuLV LTR with myeloproliferative sarcoma virus enhancer (see Challita et al. (1995) J. Virol. 69(2):748-755). In some embodiments, the promoter is a tissue-specific promoter. In some instances, the promoter drives expression only in a specific cell type (e.g., a T cell or B cell or NK cell specific promoter).

In some embodiments, the promoter is a viral promoter. In some embodiments, the promoter is a non-viral promoter. In some cases, the promoter is selected from among human elongation factor 1 alpha (EF1α) promoter (such as set forth in SEQ ID NO:77 or 118) or a modified form thereof (EF1α promoter with HTLV1 enhancer; such as set forth in SEQ ID NO:119) or the MND promoter (such as set forth in SEQ ID NO:186). In some embodiments, the polynucleotide does not include a heterologous or exogenous regulatory element, e.g., a promoter. In some embodiments, the promoter is a bidirectional promoter (see, e.g., WO2016/022994).

In some embodiments, transgene sequences may also include splice acceptor sequences. Exemplary known splice acceptor site sequences include, e.g., CTGACCTCTTCTCTTCCTCCCACAG (SEQ ID NO:78) (from the human HBB gene) and TTTCTCTCCACAG (SEQ ID NO:79) (from the human IgG gene).

In some embodiments, the transgene sequences may also include sequences required for transcription termination and/or polyadenylation signal. In some aspects, exemplary polyadenylation signal is selected from SV40, hGH, BGH, and rbGlob transcription termination sequence and/or polyadenylation signal. In some embodiments, the transgene includes an SV40 polyadenylation signal. In some embodiments, if present within the transgene, the transcription termination sequence and/or polyadenylation signal is typically the most 3′ sequence within the transgene, and is linked to one of the homology arm. In some aspects, the transgene sequence does not comprise a sequence encoding a 3′ UTR or a transcription terminator. In some embodiments, upon integration of the transgene into the endogenous TGFBR2 locus, the transgene is integrated upstream of the 3′ UTR and/or the transcription terminator of the endogenous TGFBR2 locus, such that the message encoding the recombinant receptor contains a 3′ UTR of the endogenous TGFBR2 locus, e.g., from the open reading frame or partial sequence thereof of the endogenous TGFBR2 locus. Thus, in some embodiments, upon integration of the transgene sequences encoding a portion of the recombinant receptor, the nucleic acid sequences encoding the recombinant receptor is operably linked to be under the control of 3′ UTR, transcription terminator and/or other regulatory elements of the endogenous TGFBR2 locus.

(vi) Exemplary Transgene Sequences

In some embodiments, an exemplary transgene includes, in 5′ to 3′ order, sequence of nucleotides encoding each encoding: a transmembrane domain (or a membrane association domain) and an intracellular region. In some embodiments, an exemplary transgene includes, in 5′ to 3′ order, sequence of nucleotides encoding each encoding: an extracellular region, a transmembrane domain and an intracellular region.

In some embodiments, the encoded recombinant receptor is a CAR, and an exemplary transgene sequence comprises, in 5′ to 3′ direction, sequence of nucleotides each encoding: a signal peptide, an extracellular binding domain, a spacer, a transmembrane domain and an intracellular region comprising a primary signaling domain or region and/or a co-stimulatory signaling domain. In some embodiments, an exemplary transgene sequence comprises, in 5′ to 3′ direction, sequence of nucleotides each encoding: a signal peptide, an extracellular binding domain, a spacer, a transmembrane domain and one or more costimulatory signaling domains. In some embodiments, an exemplary transgene sequence comprises, in 5′ to 3′ direction, sequence of nucleotides each encoding: a signal peptide, an extracellular binding domain, a spacer, a transmembrane domain and one or more costimulatory signaling domains and primary signaling domain or region.

In some embodiments, an exemplary transgene sequence comprises, in 5′ to 3′ direction, sequence of nucleotides each encoding: a transmembrane domain (or a membrane association domain), an intracellular multimerization domain, optionally one or more costimulatory signaling domain(s), and a primary signaling domain or region. In some embodiments, an exemplary transgene sequence comprises, in 5′ to 3′ direction, sequence of nucleotides each encoding: an extracellular multimerization domain, a transmembrane domain, optionally one or more costimulatory signaling domain(s), and a primary signaling domain or region.

In some embodiments, the transgene sequence comprises, in order a sequence of nucleotides encoding an extracellular binding domain, optionally an scFv; a spacer, optionally comprising a sequence from a human immunoglobulin hinge, optionally from IgG1, IgG2 or IgG4 or a modified version thereof, optionally further comprising a C_{H}2 region and/or a C_{H}3 region; and a transmembrane domain, optionally from human CD28; a costimulatory signaling domain, optionally from human 4-1BB; and an intracellular signaling region, optionally a CD3ζ chain or a portion thereof. In some embodiments, the encoded intracellular region of the recombinant receptor comprises, from its N to C terminus in order: the one or more costimulatory signaling domain(s) and a primary signaling domain or region, such as containing a CD3zeta chain or a fragment thereof.

In some embodiments, an exemplary transgene includes, in 5′ to 3′ order, sequence of nucleotides encoding each encoding: a transmembrane domain (or a membrane association domain) and an intracellular region. In some embodiments, an exemplary transgene includes, in 5′ to 3′ order, sequence of nucleotides encoding each encoding: an extracellular region, a transmembrane domain and an intracellular region.

In some embodiments, an exemplary transgene sequence encodes all or a portion of a TCRα chain. In some embodiments, an exemplary transgene sequence encodes all or a portion of a TCRβ chain. In some embodiments, an exemplary transgene sequence encodes all or a portion of both a TCRα chain and a TCRβ chain. In some embodiments, the encoded recombinant receptor is a recombinant T cell receptor (TCR) and an exemplary transgene includes, in 5′ to 3′ order, [TCRβ chain]-[linker or multicistronic element]-[TCRα chain]. In some embodiments, the encoded recombinant receptor is a recombinant TCR and an exemplary transgene includes, in 5′ to 3′ order, [TCRα chain]-[linker or multicistronic element]-[TCRβ chain].

In some embodiments, the exemplary transgene sequences can also comprise a multicistronic element, e.g., a 2A element or an internal ribosome entry site (IRES), and/or a regulatory or control element, e.g., a promoter, placed 5′ of the sequences encoding the signal peptide and/or the extracellular region. In some embodiments, the exemplary transgene sequences can also comprise additional sequences, e.g., sequence of nucleotides encoding one or more additional molecules, such as a marker, an additional recombinant receptor, an antibody or an antigen-binding fragment thereof, an immunomodulatory molecule, a ligand, a cytokine or a chemokine. In some aspects, the sequences encoding one or more other molecules and the sequence of nucleotides encoding regions or domains of the recombinant receptor are separated by regulatory sequences, such as a 2A ribosome skipping element and/or promoter sequences. In some aspects, in the exemplary transgene, the sequence of nucleotides encoding one or more additional molecules is placed 5′ of the sequences encoding the signal peptide and/or the extracellular region. In some embodiments, the sequence of nucleotides encoding one or more additional molecules is placed between the multicistronic element and/or regulatory or control element, and the sequence of nucleotides encoding regions or domains of the recombinant receptor. In some embodiments, the sequence of nucleotides encoding one or more additional molecules is placed between two elements and/or regulatory or control elements. In some embodiments, an exemplary transgene sequence comprises, in 5′ to 3′ direction: a multicistronic element and/or a regulatory element, a sequence of nucleotides encoding an additional molecule, a multicistronic element and/or a regulatory element, a signal peptide, nucleic acid sequence encoding regions or domains of the recombinant receptor (e.g., extracellular region, transmembrane domain, intracellular region).

b. Homology Arms

In some embodiments, the template polynucleotide contains one or more homology sequences (also called “homology arms”) on the 5′ and/or 3′ ends, linked to or surrounding the transgene sequences encoding one or more chains of a recombinant receptor or a portion thereof. In some embodiments, the one or more homology arms include the 5′ and/or 3′ homology arms. The homology arms allow the DNA repair mechanisms, e.g., homologous recombination machinery, to recognize the homology and use the template polynucleotide as a template for repair, and the nucleic acid sequence between the homology arms are copied into the DNA being repaired, effectively inserting or integrating the transgene sequences into the target site of integration in the genome between the location of the homology.

In some aspects, upon integration of the transgene sequences, the entire recombinant receptor is encoded by the transgene sequences, and the entire coding sequence or a portion of the coding sequences of the endogenous TGFBR2 locus is deleted. In some embodiments, the transgene sequence comprises a sequence of nucleotides that is in-frame with one or more exons of the open reading frame of the TGFBR2 locus comprised in the one or more homology arm(s). In some aspects, the entire recombinant receptor is encoded by the transgene sequences, and only a portion of the TGFBR2 locus is deleted, and the remaining portion of the endogenous TGFBR2 locus is expressed. In some aspects, the remaining portion of the TGFBR2 locus that is expressed, in some cases, encodes a dominant negative form of TGFBRII.

In some embodiments, the homology arm sequences include sequences that are homologous to the genomic sequences surrounding the genetic disruption, e.g., a target site within the TGFBR2 locus. In some embodiments, the template polynucleotide comprises the following components: [5′ homology arm]-[transgene sequences (exogenous or heterologous nucleic acid sequences, e.g., encoding a one or more chains of a recombinant receptor or a portion thereof)]-[3′ homology arm]. In some embodiments, the 5′ homology arm sequences include contiguous sequences that are homologous to sequences located near the genetic disruption on the 5′ side. In some embodiments, the 3′ homology arm sequences include contiguous sequences that are homologous to sequences located near the genetic disruption on the 3′ side. In some aspects, the target site is determined by targeting of the one or more agent(s) capable of introducing a genetic disruption, e.g., Cas9 and gRNA targeting a specific site within the TGFBR2 locus.

In some aspects, the transgene sequences within the template polynucleotide can be used to guide the location of target sites and/or homology arms. In some aspects, the target site of genetic disruption can be used as a guide to design template polynucleotides and/or homology arms used for HDR. In some embodiments, the genetic disruption can be targeted near a desired site of targeted integration of transgene sequences. In some aspects, the homology arms are designed to target integration within an exon of the open reading frame of the endogenous TGFBR2 locus, and the homology arm sequences are determined based on the desired location of integration surrounding the genetic disruption, including exon and intron sequences surrounding the genetic disruption. In some embodiments, the location of the target site, relative location of the one or more homology arm(s), and the transgene (exogenous nucleic acid sequence) for insertion can be designed depending on the requirement for efficient targeting and the length of the template polynucleotide or vector that can be used. In some aspects, the homology arms are designed to target integration within an intron of the open reading frame of the TGFBR2 locus. In some aspects, the homology arms are designed to target integration within an exon of the open reading frame of the TGFBR2 locus.

In some aspects, the target integration site (site for targeted integration) within the TGFBR2 locus is located within an open reading frame at the endogenous TGFBR2 locus. In some embodiments, the target integration site is at or near any of the target sites described herein, e.g., in Section I.A. In some aspects, the target location for integration is at or around the target site for genetic disruption, e.g., within less than 500, 450, 400, 350, 300, 250, 200, 150, 100 or 50 bp of the target site for genetic disruption.

In some aspects, the target integration site is within an exon of the open reading frame of the endogenous TGFBR2 locus. In some aspects, the target integration site is within an intron of the open reading frame of the TGFBR2 locus. In some aspects, the target integration site is within a regulatory or control element, e.g., a promoter, of the TGFBR2 locus. In some embodiments, the target integration site is within or in close proximity to exons corresponding to early coding region, e.g., exon 1, 2, 3, 4 or 5 of the open reading frame of the endogenous TGFBR2 locus, or including sequence immediately following a transcription start site, within exon 1, 2, 3, 4 or 5 (such as described in Table 1 or 2 herein), or within less than 500, 450, 400, 350, 300, 250, 200, 150, 100 or 50 bp of exon 1, 2, 3, 4 or 5. In some embodiments, the integration is targeted at or near exon 2 of the endogenous TGFBR2 locus, or within less than 500, 450, 400, 350, 300, 250, 200, 150, 100 or 50 bp of exon 2. In some aspects, the target integration site is at or near exon 1 of the endogenous TGFBR2 locus, e.g., within less than 500, 450, 400, 350, 300, 250, 200, 150, 100 or 50 bp of exon 1. In some embodiments, the target integration site is at or near exon 2 of the endogenous TGFBR2 locus, or within less than 500, 450, 400, 350, 300, 250, 200, 150, 100 or 50 bp of exon 2. In some aspects, the target integration site is at or near exon 3 of the endogenous TGFBR2 locus, e.g., within less than 500, 450, 400, 350, 300, 250, 200, 150, 100 or 50 bp of exon 3. In some aspects, the target integration site is at or near exon 4 of the endogenous TGFBR2 locus, e.g., within less than 500, 450, 400, 350, 300, 250, 200, 150, 100 or 50 bp of exon 4. In some aspects, the target integration site is at or near exon 5 of the endogenous TGFBR2 locus, e.g., within less than 500, 450, 400, 350, 300, 250, 200, 150, 100 or 50 bp of exon 5. In some aspects, the target integration site is within a regulatory or control element, e.g., a promoter, of the TGFBR2 locus.

In some embodiments, the 5′ homology arm sequences include contiguous sequences of approximately 10, 20, 30, 40, 50, 100, 200, 300, 400, 500, 600, 700, 800, 900, 1000, 1500, 2000, 3000, 4000, or 5000 base pairs 5′ of the target site for genetic disruption, starting near the target site at the endogenous TGFBR2 locus. In some embodiments, the 3′ homology arm sequences include contiguous sequences of approximately 10, 20, 30, 40, 50, 100, 200, 300, 400, 500, 600, 700, 800, 900, 1000, 1500, 2000, 3000, 4000, or 5000 base pairs 3′ of the target site for genetic disruption, starting near the target site at the endogenous TGFBR2 locus. Thus, upon integration via HDR, the transgene sequence is targeted for integration at or near the target site for genetic disruption, e.g., a target site within an exon or intron of the endogenous TGFBR2 locus.

In some aspects, the homology arms contain sequences that are homologous to a portion of an open reading frame sequence at the endogenous TGFBR2 locus. In some aspects, the homology arm sequences contain sequences homologous to contiguous portion of an open reading frame sequence, including exons and introns, at the endogenous TGFBR2 locus. In some aspects, the homology arm contains sequences that are identical to a contiguous portion of an open reading frame sequence, including exons and introns, at the endogenous TGFBR2 locus.

In some embodiments, the template polynucleotide contains homology arms for targeting integration of the transgene sequences at the endogenous TGFBR2 locus (exemplary genomic locus sequence described in Table 1 or 2 herein; exemplary human TGFBRII mRNA sequence set forth in SEQ ID NO:61, NCBI Reference Sequence: NM_003242.5 or SEQ ID NO:62, NCBI Reference Sequence: NM_001024847.2). In some embodiments, the genetic disruption is introduced using any of the agents for genetic disruption, e.g., targeted nucleases and/or gRNAs described herein. In some embodiments, the template polynucleotide comprises about 500 to 1000, e.g., 500 to 900 or 600 to 700, base pairs of homology on either side of the genetic disruption introduced by the targeted nucleases and/or gRNAs. In some embodiments, the template polynucleotide comprises about 500, 600, 700, 800, 900 or 1000 base pairs of 5′ homology arm sequences, which is homologous to 500, 600, 700, 800, 900 or 1000 base pairs of sequences 5′ of the genetic disruption at a TGFBR2 locus, the transgene, and about 500, 600, 700, 800, 900 or 1000 base pairs of 3′ homology arm sequences, which is homologous to 500, 600, 700, 800, 900 or 1000 base pairs of sequences 3′ of the genetic disruption at a TGFBR2 locus.

In some aspects, the boundary between the transgene and the one or more homology arm sequences, is designed such that upon HDR and targeted integration of the transgene sequences, the sequences within the transgene that encode one or more polypeptide, e.g., chain(s), domain(s) or region(s) of a recombinant receptor, is integrated in-frame with one or more exons of the open reading frame sequence at the endogenous TGFBR2 locus, and/or generates an in-frame fusion of the transgene that encode a polypeptide and one or more exons of the open reading frame sequence at the endogenous TGFBR2 locus. In some embodiments, a dominant negative (DN) form of the TGFBRII polypeptide is encoded by the nucleic acid sequences of the endogenous open reading frame, and a polypeptide of the recombinant receptor or a portion thereof is encoded by the integrated transgene sequences, optionally, separated by a multicistronic element, such as a 2A element.

In some embodiments, the one or more homology arm sequences include sequences that are homologous, substantially identical or identical to sequences that surround or flank the target site that are within an open reading frame sequence at the endogenous TGFBR2 locus. In some aspects, the one or more homology arm sequences contain introns and exons of a partial sequence of an open reading frame at the endogenous TGFBR2 locus. In some aspects, the boundary of the 5′ homology arm sequence and the transgene is such that, in a case of a transgene that does not contain a heterologous promoter, the coding portion of the transgene sequence is fused in-frame with an upstream exon or a portion thereof, e.g., exon 1, 2, 3, 4 or 5, depending on the location of targeted integration, of the open reading frame of the endogenous TGFBR2 locus.

In some aspects, the boundary of the 5′ homology arm sequence and the transgene is such that, the upstream exons or a portion thereof, e.g., exons 1, 2, 3, 4, or 5, of the open reading frame of the endogenous TGFBR2 locus, is fused in-frame with the coding portions of the transgene sequence. Thus, upon targeted integration, transcription and translation, the encoded recombinant receptor that is a contiguous polypeptide is produced, from a fusion DNA sequence of an open reading frame sequence of the endogenous TGFBR2 locus and the transgene. In some aspects, the upstream exons or a portion thereof encode a dominant negative form of the TGFBRII polypeptide. In some aspects, upon targeted integration, a multicistronic element, e.g., a 2A element or an internal ribosome entry site (IRES) separates the open reading frame sequence of the endogenous TGFBR2 locus and the transgene sequence encoding the recombinant receptor. In some aspects, when expressed and translated from the modified TGFBR2 locus, the polypeptide is cleaved to generate a dominant negative form of the TGFBRII polypeptide and a recombinant receptor.

In some embodiments, exemplary 5′ homology arm for targeting integration at the endogenous TGFBR2 locus comprises the sequence set forth in SEQ ID NO:69-71, or a sequence that exhibits at least 85%, 86%, 87%, 88%, 89%, 90%, 91%, 92%, 93%, 94%, 95%, 96%, 97%, 98%, 99% or more sequence identity to SEQ ID NO: 69-71 or a partial sequence thereof. In some aspects, exemplary 5′ homology arm for targeting integration of the transgene at the endogenous TGFBR2 locus and generating a modified TGFBR2 locus encoding a dominant negative TGFBRII comprises the sequence set forth in SEQ ID NO:70, or a sequence that exhibits at least 85%, 86%, 87%, 88%, 89%, 90%, 91%, 92%, 93%, 94%, 95%, 96%, 97%, 98%, 99% or more sequence identity to SEQ ID NO:70 or a partial sequence thereof.

In some embodiments, exemplary 3′ homology arm for targeting integration at the endogenous TGFBR2 locus comprises the sequence set forth in SEQ ID NO:72, or a sequence that exhibits at least 85%, 86%, 87%, 88%, 89%, 90%, 91%, 92%, 93%, 94%, 95%, 96%, 97%, 98%, 99% or more sequence identity to SEQ ID NO:72 or a partial sequence thereof.

In some aspects, the target site can determine the relative location and sequences of the homology arms. The homology arm can typically extend at least as far as the region in which end resection by the DNA repair mechanism can occur after the genetic disruption, e.g., DSB, is introduced, e.g., in order to allow the resected single stranded overhang to find a complementary region within the template polynucleotide. The overall length could be limited by parameters such as plasmid size, viral packaging limits or construct size limit.

In some embodiments, the homology arm comprises about 500 to 1000, e.g., 600 to 900 or 700 to 800, base pairs of homology on either side of the target site at the endogenous gene. In some embodiments, the homology arm comprises about at least or less than or about 200, 300, 400, 500, 600, 700, 800, 900 or 1000 base pairs homology 5′ of the target site, 3′ of the target site, or both 5′ and 3′ of the target site at TGFBR2 locus.

In some embodiments, the homology arm comprises at or about 10, 20, 30, 40, 50, 100, 200, 300, 400, 500, 600, 700, 800, 900, 1000, 1500, 2000, 3000, 4000, or 5000 base pairs homology 3′ of the target site at TGFBR2 locus. In some embodiments, the homology arm comprises at or about 100 to 500, 200 to 400 or 250 to 350, base pairs homology 3′ of the transgene and/or target site at TGFBR2 locus. In some embodiments, the homology arm comprises less than about 100, 90, 80, 70, 60, 50, 40, 30, 20, 15, or 10 base pairs homology 5′ of the target site at TGFBR2 locus.

In some embodiments, the homology arm comprises at or about 10, 20, 30, 40, 50, 100, 200, 300, 400, 500, 600, 700, 800, 900, 1000, 1500, 2000, 3000, 4000, or 5000 base pairs homology 5′ of the target site at TGFBR2 locus. In some embodiments, the homology arm comprises at or about 100 to 500, 200 to 400 or 250 to 350, base pairs homology 5′ of the transgene and/or target site at TGFBR2 locus. In some embodiments, the homology arm comprises less than about 100, 90, 80, 70, 60, 50, 40, 30, 20, 15, or 10 base pairs homology 3′ of the target site at TGFBR2 locus.

In some embodiments, the 3′ end of the 5′ homology arm is the position next to the 5′ end of the transgene. In some embodiments, the 5′ homology arm can extend at least at or about 10, 20, 30, 40, 50, 100, 200, 300, 400, 500, 600, 700, 800, 900, 1000, 1500, 2000, 3000, 4000, or 5000 nucleotides 5′ from the 5′ end of the transgene.

In some embodiments, the 5′ end of the 3′ homology arm is the position next to the 3′ end of the transgene. In some embodiments, the 3′ homology arm can extend at least at or about 10, 20, 30, 40, 50, 100, 200, 300, 400, 500, 600, 700, 800, 900, 1000, 1500, 2000, 3000, 4000, or 5000 nucleotides 3′ from the 3′ end of the transgene.

In some embodiments, for targeted insertion, the homology arms, e.g., the 5′ and 3′ the homology arms, may each comprise about 1000 base pairs (bp) of sequence flanking the most distal target sites (e.g., 1000 bp of sequence on either side of the mutation).

Exemplary homology arm lengths include at least at or about 50, 100, 200, 250, 300, 400, 500, 600, 700, 750, 800, 900, 1000, 2000, 3000, 4000, or 5000 nucleotides. In some embodiments, the homology arm length is at or about 50-100, 100-250, 250-500, 500-750, 750-1000, 1000-2000, 2000-3000, 3000-4000, or 4000-5000 nucleotides. Exemplary homology arm lengths include less than or less than about or is or is about 50, 100, 200, 250, 300, 400, 500, 600, 700, 750, 800, 900, 1000, 2000, 3000, 4000, or 5000 nucleotides. In some embodiments, the homology arm length is at or about 50-100, 100-250, 250-500, 500-750, 750-1000, 1000-2000, 2000-3000, 3000-4000, or 4000-5000 nucleotides. Exemplary homology arm lengths include from at or about 100 to at or about 1000 nucleotides, from at or about 100 to at or about 750 nucleotides, from at or about 100 to at or about 600 nucleotides, from at or about 100 to at or about 400 nucleotides, from at or about 100 to at or about 300 nucleotides, from at or about 100 to at or about 200 nucleotides, from at or about 200 to at or about 1000 nucleotides, from at or about 200 to at or about 750 nucleotides, from at or about 200 to at or about 600 nucleotides, from at or about 200 to at or about 400 nucleotides, from at or about 200 to at or about 300 nucleotides, from at or about 300 to at or about 1000 nucleotides, from at or about 300 to at or about 750 nucleotides, from at or about 300 to at or about 600 nucleotides, from at or about 300 to at or about 400 nucleotides, from at or about 400 to at or about 1000 nucleotides, from at or about 400 to at or about 750 nucleotides, from at or about 400 to at or about 600 nucleotides, from at or about 600 to at or about 1000 nucleotides, from at or about 600 to at or about 750 nucleotides or 750 to at or about 1000 nucleotides.

In some of any such embodiments, the transgene is integrated by a template polynucleotide introduced into each of a plurality of T cells. In particular embodiments, the template polynucleotide comprises the structure [5′ homology arm]-[transgene]-[3′ homology arm]. In certain embodiments, the 5′ homology arm and the 3′ homology arm comprises nucleic acid sequences homologous to nucleic acid sequences surrounding the at least at or about one target site. In some embodiments, the 5′ homology arm comprises nucleic acid sequences that are homologous to nucleic acid sequences 5′ of the target site. In particular embodiments, the 3′ homology arm comprises nucleic acid sequences that are homologous to nucleic acid sequences 3′ of the target site. In certain embodiments, the 5′ homology arm and the 3′ homology arm independently are at least at or about or at least at or about 10, 20, 30, 40, 50, 100, 200, 300, 400, 500, 600, 700, 800, 900, 1000, 1500, or 2000 nucleotides, or less than or less than about 10, 20, 30, 40, 50, 100, 200, 300, 400, 500, 600, 700, 800, 900, 1000, 1500, or 2000 nucleotides. In some embodiments, the 5′ homology arm and the 3′ homology arm independently are between at or about 50 and at or about 100, 100 and at or about 250, 250 and at or about 500, 500 and at or about 750, 750 and at or about 1000, 1000 and at or about 2000 nucleotides. In some of any such embodiments, the 5′ homology arm and the 3′ homology arm independently are between at or about 50 and at or about 100 nucleotides in length, at or about 100 and at or about 250 nucleotides in length, at or about 250 and at or about 500 nucleotides in length, at or about 500 and at or about 750 nucleotides in length, at or about 750 and at or about 1000 nucleotides in length, or at or about 1000 and at or about 2000 nucleotides in length.

In particular embodiments, the 5′ homology arm and the 3′ homology arm independently are from at or about 100 to at or about 1000 nucleotides, from at or about 100 to at or about 750 nucleotides, from at or about 100 to at or about 600 nucleotides, from at or about 100 to at or about 400 nucleotides, from at or about 100 to at or about 300 nucleotides, from at or about 100 to at or about 200 nucleotides, from at or about 200 to at or about 1000 nucleotides, from at or about 200 to at or about 750 nucleotides, from at or about 200 to at or about 600 nucleotides, from at or about 200 to at or about 400 nucleotides, from at or about 200 to at or about 300 nucleotides, from at or about 300 to at or about 1000 nucleotides, from at or about 300 to at or about 750 nucleotides, from at or about 300 to at or about 600 nucleotides, from at or about 300 to at or about 400 nucleotides, from at or about 400 to at or about 1000 nucleotides, from at or about 400 to at or about 750 nucleotides, from at or about 400 to at or about 600 nucleotides, from at or about 600 to at or about 1000 nucleotides, from at or about 600 to at or about 750 nucleotides or from at or about 750 to at or about 1000 nucleotides. In particular embodiments, the 5′ homology arm and the 3′ homology arm independently are from at or about 100 to at or about at or about 1000 nucleotides, from at or about 100 to at or about 750 nucleotides, from at or about 100 to at or about 600 nucleotides, from at or about 100 to at or about 400 nucleotides, from at or about 100 to at or about 300 nucleotides, from at or about 100 to at or about 200 nucleotides, from at or about 200 to at or about 1000 nucleotides, from at or about 200 to at or about 750 nucleotides, from at or about 200 to at or about 600 nucleotides, from at or about 200 to at or about 400 nucleotides, from at or about 200 to at or about 300 nucleotides, from at or about 300 to at or about 1000 nucleotides, from at or about 300 to at or about 750 nucleotides, from at or about 300 to at or about 600 nucleotides, from at or about 300 to at or about 400 nucleotides, from at or about 400 to at or about 1000 nucleotides, from at or about 400 to at or about 750 nucleotides, from at or about 400 to at or about 600 nucleotides, from at or about 600 to at or about 1000 nucleotides, from at or about 600 to at or about 750 nucleotides or from at or about 750 to at or about 1000 nucleotides in length. In some embodiments, the 5′ homology arm and the 3′ homology arm independently are at or about 200, 300, 400, 500, 600, 700 or 800 nucleotides in length, or any value between any of the foregoing. In some embodiments, the 5′ homology arm and the 3′ homology arm independently are greater than at or about 300 nucleotides in length, optionally wherein the 5′ homology arm and the 3′ homology arm independently are at or about 400, 500 or 600 nucleotides in length or any value between any of the foregoing. In some embodiments, the 5′ homology arm and the 3′ homology arm independently are greater than at or about 300 nucleotides in length.

In some embodiments, one or more of the homology arms contain a sequence of nucleotides are homologous to sequences that encode a TGFBRII or a fragment thereof. In some embodiments, one or more homology arms are connected or linked in frame with the transgene sequences encoding a recombinant receptor or a portion thereof.

In some embodiments, alternative HDR is employed. In some embodiments, alternative HDR proceeds more efficiently when the template polynucleotide has extended homology 5′ to the target site (i.e., in the 5′ direction of the target site strand). Accordingly, in some embodiments, the template polynucleotide has a longer homology arm and a shorter homology arm, wherein the longer homology arm can anneal 5′ of the target site. In some embodiments, the arm that can anneal 5′ to the target site is at least 25, 50, 75, 100, 125, 150, 175, or 200, 300, 400, 500, 600, 700, 800, 900, 1000, 1500, 2000, 3000, 4000, or 5000 nucleotides from the target site or the 5′ or 3′ end of the transgene. In some embodiments, the arm that can anneal 5′ to the target site is at least 10%, 20%, 30%, 40%, or 50% longer than the arm that can anneal 3′ to the target site. In some embodiments, the arm that can anneal 5′ to the target site is at least 2×, 3×, 4×, or 5× longer than the arm that can anneal 3′ to the target site. Depending on whether a ssDNA template can anneal to the intact strand or the targeted strand, the homology arm that anneals 5′ to the target site may be at the 5′ end of the ssDNA template or the 3′ end of the ssDNA template, respectively.

Similarly, in some embodiments, the template polynucleotide has a 5′ homology arm, a transgene, and a 3′ homology arm, such that the template polynucleotide contains extended homology to the 5′ of the target site. For example, the 5′ homology arm and the 3′ homology arm may be substantially the same length, but the transgene may extend farther 5′ of the target site than 3′ of the target site. In some embodiments, the homology arm extends at least 10%, 20%, 30%, 40%, 50%, 2×, 3×, 4×, or 5× further to the 5′ end of the target site than the 3′ end of the target site.

In some embodiments alternative HDR proceeds more efficiently when the template polynucleotide is centered on the target site. Accordingly, in some embodiments, the template polynucleotide has two homology arms that are essentially the same size. In some embodiments, the first homology arm (e.g., 5′ homology arm) of a template polynucleotide may have a length that is within 10%, 9%, 8%, 7%, 6%, 5%, 4%, 3%, 2%, or 1% of the second homology arm (e.g., 3′ homology arm) of the template polynucleotide.

Similarly, in some embodiments, the template polynucleotide has a 5′ homology arm, a transgene, and a 3′ homology arm, such that the template polynucleotide extends substantially the same distance on either side of the target site. For example, the homology arms may have different lengths, but the transgene may be selected to compensate for this. For example, the transgene may extend further 5′ from the target site than it does 3′ of the target site, but the homology arm 5′ of the target site is shorter than the homology arm 3′ of the target site, to compensate. The converse is also possible, e.g., that the transgene may extend further 3′ from the target site than it does 5′ of the target site, but the homology arm 3′ of the target site is shorter than the homology arm 5′ of the target site, to compensate.

In some embodiments, the length of the template polynucleotide, including the transgene sequence and the one or more homology arms, is between or between about 1000 to about 20,000 base pairs, such as about 1000, 1500, 2000, 2500, 3000, 3500, 4000, 4500, 5000, 6000, 7000, 8000, 9000, 10000, 11000, 12000, 13000, 14000, 15000, 16000, 17000, 18000, 19000 or 20000 base pairs. In some embodiments, the length of the template polynucleotide is limited by the maximum length of polynucleotide that can be prepared, synthesized or assembled and/or introduced into the cell or the capacity of the viral vector, and the type of polynucleotide or vector. In some aspects, the limited capacity of the template polynucleotide can determine the length of the transgene sequences and/or the one or more homology arms. In some aspects, the combined total length of the transgene sequences and the one or more homology arms must be within the maximum length or capacity of the polynucleotide or vector. For example, in some aspects, the transgene portion of the template polynucleotide is about 1000, 1500, 2000, 2500, 3000, 3500 or 4000 base pairs, and if the maximum length of the template polynucleotide is about 5000 base pairs, the remaining portion of the sequence can be divided among the one or more homology arms, e.g., such that the 3′ or 5′ homology arms can be approximately 500, 750, 1000, 1250, 1500, 1750 or 2000 base pairs.

3. Delivery of Template Polynucleotides

In some embodiments, the polynucleotide, e.g., a polynucleotide such as a template polynucleotide containing transgene sequences encoding the one or more chains of a recombinant receptor (for example, described in Section I.B.2 herein), are introduced into the cells in nucleotide form, e.g., as a polynucleotide or a vector. In particular embodiments, the polynucleotide contains a transgene that encodes the one or more chains of a recombinant receptor or a portion thereof and one or more homology arms, and can be introduced into the cell for homology-directed repair (HDR)-mediated integration of the transgene sequences.

In some aspects, the provided embodiments genetic engineering of cells, by the introduction of one or more agent(s) or components thereof capable of inducing a genetic disruption and a template polynucleotide, to induce (HDR and targeted integration of the transgene sequences. In some aspects, the one or more agent(s) and the template polynucleotide are delivered simultaneously. In some aspects, the one or more agent(s) and the template polynucleotide are delivered sequentially. In some embodiments, the one or more agent(s) are delivered prior to the delivery of the polynucleotide.

In some embodiments, the template polynucleotide is introduced into the cell for engineering, in addition to the agent(s) capable of inducing a targeted genetic disruption, e.g., nuclease and/or gRNAs. In some embodiments, the template polynucleotide(s) may be delivered prior to, simultaneously or after one or more components of the agent(s) capable of inducing a targeted genetic disruption is introduced into a cell. In some embodiments, the template polynucleotide(s) are delivered simultaneously with the agents. In some embodiments, the template polynucleotides are delivered prior to the agents, for example, seconds to hours to days before the template polynucleotides, including, but not limited to, 1 to 60 minutes (or any time therebetween) before the agents, 1 to 24 hours (or any time therebetween) before the agents or more than 24 hours before the agents. In some embodiments, the template polynucleotides are delivered after the agents, seconds to hours to days after the template polynucleotides, including immediately after delivery of the agent, e.g., between 30 seconds to 4 hours, such as about 30 seconds, 1 minute, 2 minutes, 3 minutes, 4 minutes, 5 minutes, 6 minutes, 6 minutes, 8 minutes, 9 minutes, 10 minutes, 15 minutes, 20 minutes, 30 minutes, 40 minutes, 50 minutes, 60 minutes, 90 minutes, 2 hours, 3 hours or 4 hours after delivery of the agents and/or preferably within 4 hours of delivery of the agents. In some embodiments, the template polynucleotide is delivered more than 4 hours after delivery of the agents.

In some embodiments, the template polynucleotides may be delivered using the same delivery systems as the agent(s) capable of inducing a targeted genetic disruption, e.g., nuclease and/or gRNAs. In some embodiments, the template polynucleotides may be delivered using different same delivery systems as the agent(s) capable of inducing a targeted genetic disruption, e.g., nuclease and/or gRNAs. In some embodiments, the template polynucleotide is delivered simultaneously with the agent(s). In other embodiments, the template polynucleotide is delivered at a different time, before or after delivery of the agent(s). Any of the delivery method described herein in Section I.A.3 (e.g., in Tables 4 and 5) for delivery of nucleic acids in the agent(s) capable of inducing a targeted genetic disruption, e.g., nuclease and/or gRNAs, can be used to deliver the template polynucleotide.

In some embodiments, the one or more agent(s) and the template polynucleotide are delivered in the same format or method. For example, in some embodiments, the one or more agent(s) and the template polynucleotide are both comprised in a vector, e.g., viral vector. In some embodiments, the template polynucleotide is encoded on the same vector backbone, e.g. AAV genome, plasmid DNA, as the Cas9 and gRNA. In some aspects, the one or more agent(s) and the template polynucleotide are in different formats, e.g., ribonucleic acid-protein complex (RNP) for the Cas9-gRNA agent and a linear DNA for the template polynucleotide, but they are delivered using the same method.

In some embodiments, the template polynucleotide is a linear or circular nucleic acid molecule, such as a linear or circular DNA or linear RNA, and can be delivered using any of the methods described in Section I.A.3 herein (e.g., Tables 4 and 5 herein) for delivering nucleic acid molecules into the cell.

In particular embodiments, the polynucleotide, e.g., the template polynucleotide, are introduced into the cells in nucleotide form, e.g., as or within a non-viral vector. In some embodiments, the non-viral vector is or includes a polynucleotide, e.g., a DNA or RNA polynucleotide, that is suitable for transduction and/or transfection by any suitable and/or known non-viral method for gene delivery, such as but not limited to microinjection, electroporation, transient cell compression or squeezing (such as described in Lee, et al. (2012) Nano Lett 12: 6322-27), lipid-mediated transfection, peptide-mediated delivery, e.g., cell-penetrating peptides, or a combination thereof. In some embodiments, the non-viral polynucleotide is delivered into the cell by a non-viral method described herein, such as a non-viral method listed in Table 5 herein.

In some embodiments, the template polynucleotide sequence can be comprised in a vector molecule containing sequences that are not homologous to the region of interest in the genomic DNA. In some embodiments, the virus is a DNA virus (e.g., dsDNA or ssDNA virus). In some embodiments, the virus is an RNA virus (e.g., an ssRNA virus). Exemplary viral vectors/viruses include, e.g., retroviruses, lentiviruses, adenovirus, adeno-associated virus (AAV), vaccinia viruses, poxviruses, and herpes simplex viruses, or any of the viruses described elsewhere herein. A polynucleotide can be introduced into a cell as part of a vector molecule having additional sequences such as, for example, replication origins, promoters and genes encoding antibiotic resistance. Moreover, template polynucleotides can be introduced as naked nucleic acid, as nucleic acid complexed with materials such as a liposome, nanoparticle or poloxamer, or can be delivered by viruses (e.g., adenovirus, AAV, herpesvirus, retrovirus, lentivirus and integrase defective lentivirus (IDLV)).

In some embodiments, the template polynucleotide can be transferred into cells using recombinant infectious virus particles, such as, e.g., vectors derived from simian virus 40 (SV40), adenoviruses, adeno-associated virus (AAV). In some embodiments, the template polynucleotide is transferred into T cells using recombinant lentiviral vectors or retroviral vectors, such as gamma-retroviral vectors (see, e.g., Koste et al. (2014) Gene Therapy 2014 Apr. 3. doi: 10.1038/gt.2014.25; Carlens et al. (2000) Exp Hematol 28(10): 1137-46; Alonso-Camino et al. (2013) Mol Ther Nucl Acids 2, e93; Park et al., Trends Biotechnol. 2011 Nov. 29(11): 550-557 or HIV-1 derived lentiviral vectors.

In other aspects, the template polynucleotide is delivered by viral and/or non-viral gene transfer methods. In some embodiments, the template polynucleotide is delivered to the cell via an adeno associated virus (AAV). Any AAV vector can be used, including, but not limited to, AAV1, AAV2, AAV3, AAV4, AAV5, AAV6, AAV7, AAV8 and combinations thereof. In some instances, the AAV comprises LTRs that are of a heterologous serotype in comparison with the capsid serotype (e.g., AAV2 ITRs with AAV5, AAV6, or AAV8 capsids). The template polynucleotide may be delivered using the same gene transfer system as used to deliver the nuclease (including on the same vector) or may be delivered using a different delivery system that is used for the nuclease. In some embodiments, the template polynucleotide is delivered using a viral vector (e.g., AAV) and the nuclease(s) is(are) delivered in mRNA form. The cell may also be treated with one or more molecules that inhibit binding of the viral vector to a cell surface receptor as described herein prior to, simultaneously and/or after delivery of the viral vector (e.g., carrying the nuclease(s) and/or template polynucleotide).

In some embodiments, the retroviral vector has a long terminal repeat sequence (LTR), e.g., a recombinant retroviral vector derived from the Moloney murine leukemia virus (MoMLV), myeloproliferative sarcoma virus (MPSV), murine embryonic stem cell virus (MESV), murine stem cell virus (MSCV), or spleen focus forming virus (SFFV). Most retroviral vectors are derived from murine retroviruses. In some embodiments, the retroviruses include those derived from any avian or mammalian cell source. The retroviruses typically are amphotropic, meaning that they are capable of infecting host cells of several species, including humans. In one embodiment, the gene to be expressed replaces the retroviral gag, pol and/or env sequences. A number of illustrative retroviral systems have been described (e.g., U.S. Pat. Nos. 5,219,740; 6,207,453; 5,219,740; Miller and Rosman (1989) BioTechniques 7:980-990; Miller, A. D. (1990) Human Gene Therapy 1:5-14; Scarpa et al. (1991) Virology 180:849-852; Burns et al. (1993) Proc. Natl. Acad. Sci. USA 90:8033-8037; and Boris-Lawrie and Temin (1993) Cur. Opin. Genet. Develop. 3:102-109).

In some embodiments, the template polynucleotides are delivered using an AAV vector and the agent(s) capable of inducing a targeted genetic disruption, such as nuclease and/or gRNAs are delivered as a different form, such as mRNAs encoding the nucleases and/or gRNAs. In some embodiments, the template polynucleotides and nucleases are delivered using the same type of method, such as a viral vector, but on separate vectors. In some embodiments, the template polynucleotides are delivered in a different delivery system as the agents capable of inducing a genetic disruption, such as nucleases and/or gRNAs. Types or nucleic acids and vectors for delivery include any of those described in Section III herein.

In some embodiments, the template polynucleotides and nucleases may be on the same vector, for example an AAV vector (such as AAV6). In some embodiments, the template polynucleotides are delivered using an AAV vector and the agent(s) capable of inducing a targeted genetic disruption, such as nuclease and/or gRNAs are delivered as a different form, such as mRNAs encoding the nucleases and/or gRNAs. In some embodiments, the template polynucleotides and nucleases are delivered using the same type of method, such as a viral vector, but on separate vectors. In some embodiments, the template polynucleotides are delivered in a different delivery system as the agents capable of inducing a genetic disruption, such as nucleases and/or gRNAs. In some embodiments, the template polynucleotide is excised from a vector backbone in vivo, such as it is flanked by gRNA recognition sequences. In some embodiments, the template polynucleotide is on a separate polynucleotide molecule as the Cas9 and gRNA. In some embodiments, the Cas9 and the gRNA are introduced in the form of a ribonucleoprotein (RNP) complex, and the template polynucleotide is introduced as a polynucleotide molecule, such as in a vector or a linear nucleic acid molecule, such as linear DNA. Types or nucleic acids and vectors for delivery include any of those described in Section II herein.

In some embodiments, the template polynucleotide is an adenovirus vector, e.g., an AAV vector, e.g., a ssDNA molecule of a length and sequence that allows it to be packaged in an AAV capsid. The vector may be, e.g., less than 5 kb and may contain an ITR sequence that promotes packaging into the capsid. The vector may be integration-deficient. In some embodiments, the template polynucleotide comprises about 150 to 1000 nucleotides of homology on either side of the transgene and/or the target site. In some embodiments, the template polynucleotide comprises about 100, 150, 200, 300, 400, 500, 600, 700, 800, 900, 1000, 1500, or 2000 nucleotides 5′ of the target site or transgene, 3′ of the target site or transgene, or both 5′ and 3′ of the target site or transgene. In some embodiments, the template polynucleotide comprises at least 100, 150, 200, 300, 400, 500, 600, 700, 800, 900, 1000, 1500, or 2000 nucleotides 5′ of the target site or transgene, 3′ of the target site or transgene, or both 5′ and 3′ of the target site or transgene. In some embodiments, the template polynucleotide comprises at most 100, 150, 200, 300, 400, 500, 600, 700, 800, 900, 1000, 1500, or 2000 nucleotides 5′ of the target site or transgene, 3′ of the target site or transgene, or both 5′ and 3′ of the target site or transgene.

In some embodiments, the template polynucleotide is a lentiviral vector, e.g., an IDLV (integration deficiency lentivirus). In some embodiments, the template polynucleotide comprises about 500 to 1000 base pairs of homology on either side of the transgene and/or the target site. In some embodiments, the template polynucleotide comprises about 300, 400, 500, 600, 700, 800, 900, 1000, 1500, or 2000 base pairs of homology 5′ of the target site or transgene, 3′ of the target site or transgene, or both 5′ and 3′ of the target site or transgene. In some embodiments, the template polynucleotide comprises at least 300, 400, 500, 600, 700, 800, 900, 1000, 1500, or 2000 base pairs of homology 5′ of the target site or transgene, 3′ of the target site or transgene, or both 5′ and 3′ of the target site or transgene. In some embodiments, the template polynucleotide comprises no more than 300, 400, 500, 600, 700, 800, 900, 1000, 1500, or 2000 base pairs of homology 5′ of the target site or transgene, 3′ of the target site or transgene, or both 5′ and 3′ of the target site or transgene. In some embodiments, the template polynucleotide comprises one or more mutations, e.g., silent mutations, that prevent Cas9 from recognizing and cleaving the template polynucleotide. The template polynucleotide may comprise, e.g., at least 1, 2, 3, 4, 5, 10, 20, or 30 silent mutations relative to the corresponding sequence in the genome of the cell to be altered. In some embodiments, the template polynucleotide comprises at most 2, 3, 4, 5, 10, 20, 30, or 50 silent mutations relative to the corresponding sequence in the genome of the cell to be altered. In some embodiments, the cDNA comprises one or more mutations, e.g., silent mutations that prevent Cas9 from recognizing and cleaving the template polynucleotide. The template polynucleotide may comprise, e.g., at least 1, 2, 3, 4, 5, 10, 20, or 30 silent mutations relative to the corresponding sequence in the genome of the cell to be altered. In some embodiments, the template polynucleotide comprises at most 2, 3, 4, 5, 10, 20, 30, or 50 silent mutations relative to the corresponding sequence in the genome of the cell to be altered.

The double-stranded template polynucleotides described herein may include one or more non-natural bases and/or backbones. In particular, insertion of a template polynucleotide with methylated cytosines may be carried out using the methods described herein to achieve a state of transcriptional quiescence in a region of interest.

### II. NUCLEIC ACIDS, VECTORS AND DELIVERY

In some embodiments, the polynucleotide, such as a polynucleotide such as a template polynucleotide encoding one or more chains of a recombinant receptor or a portion thereof, are introduced into the cells in nucleotide form, such as a polynucleotide or a vector. In particular embodiments, the polynucleotide contains a transgene that encodes the recombinant receptor or a portion thereof. In certain embodiments, the one or more agent(s) or components thereof for genetic disruption are introduced into the cells in nucleic acid form, such as polynucleotides and/or vectors. In some embodiments, the components for engineering can be delivered in various forms using various delivery methods, including any suitable methods used for delivery of agent(s) as described in Section I.A.3 and Tables 4 and 5 herein. Also provided are one or more polynucleotides (such as nucleic acid molecules) encoding one or more components of the one or more agent(s) capable of inducing a genetic disruption (for example, any described in Section I.A herein). Also provided are one or more template polynucleotides containing transgene (for example, any described in Section I.B.2 herein), and vectors for genetically engineering cells for targeted integration of the transgene, such as a template polynucleotide or a polynucleotide encoding one or more components of the one or more agent(s) capable of inducing a genetic disruption.

In some embodiments, provided are polynucleotides, such as template polynucleotides for targeting transgene at a specific genomic target location, such as at the TGFBR2 locus. In some embodiments, provided are any template polynucleotides described in Section I.B herein. In some embodiments, the template polynucleotide contains transgene that include nucleic acid sequences that encode a recombinant receptor or a portion thereof or other polypeptides and/or factors, and homology arms for targeted integration. In some embodiments, the template polynucleotide can be contained in a vector.

In some embodiments, agents capable of inducing a genetic disruption can be encoded in one or more polynucleotides. In some embodiments, the component of the agents, such as Cas9 molecule and/or a gRNA molecule, can be encoded in one or more polynucleotides, and introduced into the cells. In some embodiments, the polynucleotide encoding one or more component of the agents can be included in a vector.

In some embodiments, a vector may comprise a sequence that encodes a Cas9 molecule and/or a gRNA molecule and/or template polynucleotides. A vector may also comprise a sequence encoding a signal peptide (such as for nuclear localization, nucleolar localization, mitochondrial localization), fused, such as to a Cas9 molecule sequence. For example, a vector may comprise a nuclear localization sequence (such as from SV40) fused to the sequence encoding the Cas9 molecule. In some embodiments, provided are vectors for genetically engineering cells for targeted integration of the transgene sequences contained in the polynucleotides, such as the template polynucleotides described in Section I.B.2.

In particular embodiments, one or more regulatory/control elements, such as a promoter, an enhancer, an intron, a polyadenylation signal, a Kozak consensus sequence, internal ribosome entry sites (IRES), a 2A sequence, and splice acceptor or donor can be included in the vectors. In some embodiments, the promoter is selected from among an RNA pol I, pol II or pol III promoter. In some embodiments, the promoter is recognized by RNA polymerase II (such as a CMV, SV40 early region or adenovirus major late promoter). In another embodiment, the promoter is recognized by RNA polymerase III (such as a U6 or H1 promoter).

In certain embodiments, the promoter is a regulated promoter (such as inducible promoter). In some embodiments, the promoter is an inducible promoter or a repressible promoter. In some embodiments, the promoter comprises a Lac operator sequence, a tetracycline operator sequence, a galactose operator sequence or a doxycycline operator sequence, or is an analog thereof or is capable of being bound by or recognized by a Lac repressor or a tetracycline repressor, or an analog thereof.

In some embodiments, the promoter is or comprises a constitutive promoter. Exemplary constitutive promoters include, e.g., simian virus 40 early promoter (SV40), cytomegalovirus immediate-early promoter (CMV), human Ubiquitin C promoter (UBC), human elongation factor 1α promoter (EF1α), mouse phosphoglycerate kinase 1 promoter (PGK), and chicken R-Actin promoter coupled with CMV early enhancer (CAGG). In some embodiments, the constitutive promoter is a synthetic or modified promoter. In some embodiments, the promoter is or comprises an MND promoter, a synthetic promoter that contains the U3 region of a modified MoMuLV LTR with myeloproliferative sarcoma virus enhancer (sequence set forth in SEQ ID NO:186; see Challita et al. (1995) J. Virol. 69(2):748-755). In some embodiments, the promoter is a tissue-specific promoter. In another embodiment, the promoter is a viral promoter. In another embodiment, the promoter is a non-viral promoter. In some embodiments, exemplary promoters can include, but are not limited to, human elongation factor 1 alpha (EF1α) promoter (such as set forth in SEQ ID NO:77 or 118) or a modified form thereof (EF1α promoter with HTLV1 enhancer; such as set forth in SEQ ID NO:119) or the MND promoter (such as set forth in SEQ ID NO:186). In some embodiments, the polynucleotide and/or vector does not include a regulatory element, e.g. promoter.

In particular embodiments, the polynucleotide, e.g., the polynucleotide encoding the recombinant receptor or a portion thereof, are introduced into the cells in nucleotide form, e.g., as or within a non-viral vector. In some embodiments, the polynucleotide is a DNA or an RNA polynucleotide. In some embodiments, the polynucleotide is a double-stranded or single-stranded polynucleotide. In some embodiments, the non-viral vector is or includes a polynucleotide, e.g., a DNA or RNA polynucleotide, that is suitable for transduction and/or transfection by any suitable and/or known non-viral method for gene delivery, such as but not limited to microinjection, electroporation, transient cell compression or squeezing (such as described in Lee, et al. (2012) Nano Lett 12: 6322-27), lipid-mediated transfection, peptide-mediated delivery, or a combination thereof. In some embodiments, the non-viral polynucleotide is delivered into the cell by a non-viral method described herein, such as a non-viral method listed in Table 5.

In some embodiments, the vector or delivery vehicle is a viral vector (e.g., for generation of recombinant viruses). In some embodiments, the virus is a DNA virus (e.g., dsDNA or ssDNA virus). In some embodiments, the virus is an RNA virus (e.g., an ssRNA virus). Exemplary viral vectors/viruses include, e.g., retroviruses, lentiviruses, adenovirus, adeno-associated virus (AAV), vaccinia viruses, poxviruses, and herpes simplex viruses, or any of the viruses described elsewhere herein.

In some embodiments, the virus infects dividing cells. In another embodiment, the virus infects non-dividing cells. In another embodiment, the virus infects both dividing and non-dividing cells. In another embodiment, the virus can integrate into the host genome. In another embodiment, the virus is engineered to have reduced immunity, e.g., in human. In another embodiment, the virus is replication-competent. In another embodiment, the virus is replication-defective, e.g., having one or more coding regions for the genes necessary for additional rounds of virion replication and/or packaging replaced with other genes or deleted. In another embodiment, the virus causes transient expression of the Cas9 molecule and/or the gRNA molecule for the purposes of transient induction of genetic disruption. In another embodiment, the virus causes long-lasting, e.g., at least 1 week, 2 weeks, 1 month, 2 months, 3 months, 6 months, 9 months, 1 year, 2 years, or permanent expression, of the Cas9 molecule and/or the gRNA molecule. The packaging capacity of the viruses may vary, e.g., from at least about 4 kb to at least about 30 kb, e.g., at least about 5 kb, 10 kb, 15 kb, 20 kb, 25 kb, 30 kb, 35 kb, 40 kb, 45 kb, or 50 kb.

In some embodiments, the polynucleotide containing the agent(s) and/or template polynucleotide is delivered by a recombinant retrovirus. In another embodiment, the retrovirus (e.g., Moloney murine leukemia virus) comprises a reverse transcriptase, e.g., that allows integration into the host genome. In some embodiments, the retrovirus is replication-competent. In another embodiment, the retrovirus is replication-defective, e.g., having one of more coding regions for the genes necessary for additional rounds of virion replication and packaging replaced with other genes, or deleted.

In some embodiments, the polynucleotide containing the agent(s) and/or template polynucleotide is delivered by a recombinant lentivirus. For example, the lentivirus is replication-defective, e.g., does not comprise one or more genes required for viral replication.

In some embodiments, the polynucleotide containing the agent(s) and/or template polynucleotide is delivered by a recombinant adenovirus. In another embodiment, the adenovirus is engineered to have reduced immunity in humans.

In some embodiments, the polynucleotide containing the agent(s) and/or template polynucleotide is delivered by a recombinant AAV. In some embodiments, the AAV can incorporate its genome into that of a host cell, e.g., a target cell as described herein. In another embodiment, the AAV is a self-complementary adeno-associated virus (scAAV), e.g., a scAAV that packages both strands which anneal together to form double stranded DNA. AAV serotypes that may be used in the disclosed methods, include AAV1, AAV2, modified AAV2 (e.g., modifications at Y444F, Y500F, Y730F and/or S662V), AAV3, modified AAV3 (e.g., modifications at Y705F, Y731F and/or T492V), AAV4, AAV5, AAV6, modified AAV6 (e.g., modifications at S663V and/or T492V), AAV7, AAV8, AAV 8.2, AAV9, AAV.rh10, modified AAV.rh10, AAV.rh32/33, modified AAV.rh32/33, AAV.rh43, modified AAV.rh43, AAV.rh64R1, modified AAV.rh64R1, and pseudotyped AAV, such as AAV2/8, AAV2/5 and AAV2/6 can also be used in the disclosed methods.

In some embodiments, the polynucleotide containing the agent(s) and/or template polynucleotide is delivered by a hybrid virus, e.g., a hybrid of one or more of the viruses described herein.

A packaging cell is used to form a virus particle that is capable of infecting a target cell. Such a cell includes a 293 cell, which can package adenovirus, and a ψ2 cell or a PA317 cell, which can package retrovirus. A viral vector used in gene therapy is usually generated by a producer cell line that packages a nucleic acid vector into a viral particle. The vector typically contains the minimal viral sequences required for packaging and subsequent integration into a host or target cell (if applicable), with other viral sequences being replaced by an expression cassette encoding the protein to be expressed, e.g., Cas9. For example, an AAV vector used in gene therapy typically only possesses inverted terminal repeat (ITR) sequences from the AAV genome which are required for packaging and gene expression in the host or target cell. The missing viral functions are supplied in trans by the packaging cell line. Henceforth, the viral DNA is packaged in a cell line, which contains a helper plasmid encoding the other AAV genes, namely rep and cap, but lacking ITR sequences. The cell line is also infected with adenovirus as a helper. The helper virus promotes replication of the AAV vector and expression of AAV genes from the helper plasmid. The helper plasmid is not packaged in significant amounts due to a lack of ITR sequences. Contamination with adenovirus can be reduced by, e.g., heat treatment to which adenovirus is more sensitive than AAV.

In some embodiments, the viral vector has the ability of cell type recognition. For example, the viral vector can be pseudotyped with a different/alternative viral envelope glycoprotein; engineered with a cell type-specific receptor (e.g., genetic modification of the viral envelope glycoproteins to incorporate targeting ligands such as a peptide ligand, a single chain antibody, a growth factor); and/or engineered to have a molecular bridge with dual specificities with one end recognizing a viral glycoprotein and the other end recognizing a moiety of the target cell surface (e.g., ligand-receptor, monoclonal antibody, avidin-biotin and chemical conjugation).

In some embodiments, the viral vector achieves cell type specific expression. For example, a tissue-specific promoter can be constructed to restrict expression of the agent capable of introducing a genetic disruption (e.g., Cas9 and gRNA) in only a specific target cell. The specificity of the vector can also be mediated by microRNA-dependent control of expression. In some embodiments, the viral vector has increased efficiency of fusion of the viral vector and a target cell membrane. For example, a fusion protein such as fusion-competent hemagglutinin (HA) can be incorporated to increase viral uptake into cells. In some embodiments, the viral vector has the ability of nuclear localization. For example, a virus that requires the breakdown of the nuclear membrane (during cell division) and therefore will not infect a non-diving cell can be altered to incorporate a nuclear localization peptide in the matrix protein of the virus thereby enabling the transduction of non-proliferating cells.

### III. ENGINEERED CELLS EXPRESSING RECOMBINANT RECEPTORS AND CELL COMPOSITIONS

Provided herein are genetically engineered cells comprising a modified TGFBR2 locus that comprises nucleic acid sequences, such as a transgene encoding one or more chains of a recombinant receptor, such as a chimeric antigen receptor (CAR), or a portion thereof. In some aspects, the modified TGFBR2 locus in the genetically engineered cell comprises exogenous nucleic acid sequences (e.g., transgene sequences) encoding one or more chains of a recombinant receptor or portion thereof, integrated into the endogenous TGFBR2 locus. In some aspects, the provided engineered cells are produced using methods described herein, e.g., involving homology-dependent repair (HDR) by employing agent(s) for inducing a genetic disruption (for example, as described in Section I.A) and template polynucleotides containing the transgene sequences for repair (for example, described in Section I.B). In some aspects, a part, e.g., a contiguous segment of the provided polynucleotides, such as any template polynucleotides described in Section I.B, can be targeted for integration at the endogenous TGFBR2 locus, to generate a cell containing a modified TGFBR2 locus comprising a nucleic acid sequence, such as a transgene encoding a recombinant receptor or a portion thereof. In some embodiments, the part of the template polynucleotide that is integrated by HDR into the endogenous TGFBR2 locus includes the transgene sequence portion, such as any described herein, for example in Section I.B, of the template polynucleotide.

In some aspects, the cells are engineered to express a recombinant receptor, such as a CAR or a recombinant T cell receptor (TCR). In some aspects, the recombinant receptor is encoded by the nucleic acid sequences present at the modified TGFBR2 locus in the engineered cells. In some aspects, the cells are generated by integrating transgene sequences encoding all or a portion of the recombinant receptor, via HDR. In some embodiments, the recombinant receptor contains a binding domain that binds to or recognizes a ligand or an antigen, e.g., an antigen associated with a disease or disorder.

In some aspects, the engineered cells are immune cells, such as T cells. In some aspects, the immune cells are engineered to express a recombinant receptor, e.g., chimeric antigen receptor or modified recombinant receptors, such as any described herein.

In some embodiments, the methods, compositions, articles of manufacture, and/or kits provided herein are useful to generate, manufacture, or produce genetically engineered cells, e.g., genetically engineered immune cells and/or T cells, that have or contain a modified TGFBR2 locus. In particular embodiments, the methods provided herein result in genetically engineered cells that have or contain a modified TGFBR2 locus. In some embodiments, the modified locus is or contains a transgene sequence, e.g., a transgene sequence described in Section I.B, integrated in an open reading frame of the endogenous TGFBR2 gene. In certain embodiments, the transgene is inserted in-frame into the open reading frame of the endogenous TGFBR2 gene, resulting in a modified TGFBR2 locus that encodes a partial TGFBRII polypeptide and a recombinant receptor or a portion thereof. In some embodiments, the partial TGFBRII polypeptide encoded by the modified locus is a dominant negative form of the TGFBRII polypeptide. In some embodiments, the recombinant receptor is a chimeric antigen receptor (CAR). In some aspects, the recombinant receptor is a recombinant T cell receptor (TCR).

In some cases, the cell is engineered to express one or more additional molecules, e.g., an additional factors and/or an accessory molecule, such as any additional molecules, including therapeutic molecules, described herein. In some embodiments, the additional molecules can include a marker, an additional recombinant receptor polypeptide chain, an antibody or an antigen-binding fragment thereof, an immunomodulatory molecule, a ligand, a cytokine or a chemokine. In some embodiments, the additional factors is a soluble molecule. In some embodiments, the additional factors is a membrane-bound molecule. In some aspects, the additional factors can be used to overcome or counteract the effect of an immunosuppressive environment, such as a tumor microenvironment (TME). In some aspects, exemplary additional molecule includes a cytokine, a cytokine receptor, a chimeric co-stimulatory receptor, a co-stimulatory ligand and other modulators of T cell function or activity. In some embodiments, the additional molecules expressed by the engineered cell include IL-7, IL-12, IL-15, CD40 ligand (CD40L), and 4-1BB ligand (4-1BBL). In some aspects, the additional molecule is an additional receptor, e.g., a membrane-bound receptor, that binds a different molecule. For example, in some embodiments, the additional molecule is a cytokine receptor or a chemokine receptor, e.g., IL-4 receptor or CCL2 receptor. In some cases, the engineered cells are called “armored CARs” or T cells redirected for universal cytokine killing (TRUCKs).

Also provided are compositions containing a plurality of the engineered cells. In some aspects, the compositions containing the engineered cells exhibit improved, uniform, homogeneous and/or stable expression and/or antigen binding by the recombinant receptor, compared to cells or cell compositions generated using other methods of engineering, such as methods in which the recombinant receptor is introduced randomly into the genome of a cell. In some embodiments, the engineered cells or the composition comprising the engineered cells can be used in therapy, e.g., adoptive cell therapy. In some embodiments, the provided cells or cell compositions can be used in any of the methods of treatment described herein or for therapeutic uses described herein.

### A. Modified TGFBR2 Locus

In some aspects, provided are genetically engineered cells comprising a modified TGFBR2 locus. In some embodiments, the modified TGFBR2 locus comprises a nucleic acid sequence encoding a recombinant receptor or a portion thereof. In some embodiments, the nucleic acid sequence comprises a transgene sequence encoding one or more chains of a recombinant receptor or a portion thereof, the transgene sequence having been integrated at the endogenous TGFBR2 locus, optionally via homology directed repair (HDR). In some aspects, the modified TGFBR2 locus can encode any one or more of the recombinant receptors described herein, for example in Section III.B, or a portion thereof, such as a domain or region thereof, or one or more chains of a multi-chain recombinant receptor described herein.

In some aspects, the modified TGFBR2 locus is generated as a result of genetic disruption and integration of transgene sequences (e.g. exogenous or heterologous nucleic acid sequences) that includes a sequence of nucleotides encoding a recombinant receptor or a portion thereof, such as via HDR methods. In some aspects, the nucleic acid sequence present at the modified TGFBR2 locus includes the transgene sequence(s), such as an exogenous sequence, integrated at a region in the endogenous TGFBR2 locus that normally would include an open reading frame encoding full length TGFBRII. In some aspects, upon targeted integration of the transgene by HDR, the genome of the cell contains a modified TGFBR2 locus, comprising a nucleic acid sequence encoding a recombinant receptor or a portion thereof and lacking all or at least a portion of the endogenous genome encoding full-length TGFBRII. In some embodiments, upon targeted integration, the modified TGFBR2 locus contains the transgene integrated into a site within the open reading frame of the endogenous TGFBR2 locus, such that the recombinant receptor is expressed from the engineered cell, and, in some cases, also a portion of TGFBRII, e.g. a partial or truncated TGFBRII.

In some embodiments, upon integration of the transgene sequences, the endogenous sequences of the TGFBR2 locus comprise a genetic disruption, such as a deletion of nucleic acid sequences encoding one or more amino acids and/or a mutation introducing a stop codon. In some embodiments, upon integration of the transgene sequences, the endogenous sequences of the TGFBR2 locus do not encode a functional TGFBRII polypeptide. In some embodiments, upon integration of the transgene sequences, the endogenous sequences of the TGFBR2 locus encode a partial TGFBRII polypeptide or a truncated TGFBRII polypeptide. In some embodiments, a partial or truncated TGFBRII polypeptide encoded by the endogenous sequences of the TGFBR2 locus is a dominant negative (DN) form of the TGFBRII polypeptide. In some aspects, a dominant negative form of the TGFBR2 includes a variant of TGFBR2 that, when expressed in a cell, can inhibit, reduce or interfere with signal transduction by the TGFβ receptor complex. In some aspects, exemplary dominant negative form of TGFBRII include a truncated TGFBRII, such as a TGFBRII that lacks all or a portion of the cytoplasmic domain. In some embodiments, dominant negative TGFBRII include those described in, e.g., Wieser et al., (1993) Mol. Cell Biol. 13(12): 7239-7247; Brand et al., (1995) JBC 270: 8274-8284; Bottinger et al., (1997) EMBO J 16(10): 2621-2633; Shah et al., (2002) Cancer Res 62:7135-7138; Bollard et al. (2002) Gene Therapy 99(9): 3179-87; and Zhang et al., (2013) Gene Therapy 20: 575-580; and Pang et al. (2013) Cancer Discov. 3(8): 936-951.

In some embodiments, the mRNA transcribed from the modified locus contains a 3′UTR that is encoded by the endogenous TGFBR2 locus and/or is identical to a 3′UTR of an mRNA that is transcribed from the endogenous TGFBR2 locus. In some embodiments, the transgene contains a ribosomal skipping element upstream, e.g., immediately upstream, of the sequence of nucleic acids encoding the portion of the CAR. In some embodiments, the mRNA encoding the CAR contains a 5′UTR that is encoded by the endogenous TGFBR2 locus and/or is identical to a 5′UTR of an mRNA that is transcribed from the endogenous TGFBR2 locus.

In some aspects, exemplary dominant negative form of TGFBRII include a TGFBRII containing a deletion of one or more amino acid residues, optionally one or more contiguous amino acid residues, in the an intracellular region of TGFBR2, e.g., including amino acid residues 188-567 of the human TGFBRII precursor sequence (isoform 1) set forth in SEQ ID NO:59, or amino acid residues 213-592 of the human TGFBRII precursor sequence (isoform 2) set forth in SEQ ID NO:60. In some aspects, an exemplary dominant negative form of TGFBRII includes an amino acid sequence corresponding to residues 22-191 of the amino acid sequence set forth in SEQ ID NO:59, or an amino acid sequence corresponding to residues 22-216 of the amino acid sequence set forth in SEQ ID NO:60, or a sequence that exhibits at least 85%, 86%, 87%, 88%, 89%, 90%, 91%, 92%, 93%, 94%, 95%, 96%, 97%, 98%, or 99% sequence identity thereto or a fragment thereof.

In certain embodiments, the transgene encodes a recombinant receptor and is inserted in-frame within an endogenous open reading frame of the TGFBR2 locus. In particular embodiments, the transcription of the modified locus results in an mRNA that encodes the recombinant receptor, such as a CAR. In some aspects, the nucleic acid sequence present in the open reading frame of the endogenous TGFBR2 locus can encode a partial or a truncated TGFBRII polypeptide, such as a dominant negative form of TGFBRII. In some embodiments, the transgene is integrated at a target site immediately downstream of and in frame with one or more exons of open reading frame of the endogenous TGFBR2 locus. In some embodiments, the transgene sequences is integrated or inserted downstream of exon 1, 2, 3 or 4 and upstream of exon 6, 7 or 8 of the open reading frame of the endogenous TGFBR2 locus (such as described in Tables 1 and 2 herein). In some embodiments, the transgene sequences is integrated or inserted downstream of exon 1, 2, 3 or 4 and upstream of exon 6 of the open reading frame of the endogenous TGFBR2 locus (such as described in Tables 1 and 2 herein). In some embodiments, the transgene sequence is downstream of exon 1 and upstream of exon 6 of the open reading frame of the endogenous TGFBR2 locus. In some embodiments, the transgene sequence is downstream of exon 3 and upstream of exon 5 of the open reading frame of the endogenous TGFBR2 locus. In some embodiments, the transgene sequence is downstream of exon 4 and upstream of exon 6 of the open reading frame of the endogenous TGFBR2 locus.

In some embodiments, the recombinant receptor encoded from the modified TGFBR2 locus is a CAR. In some embodiments, the CAR encoded by the modified TGFBR2 locus binds to and/or is capable of binding to a target antigen. In some embodiments, the target antigen is associated with, specific to, and/or expressed on a cell or tissue that is associated with a disease, disorder, or condition. In some embodiments, the CAR is capable of stimulating and/or inducing a primary activation signal in a T cell, a signaling domain of a T cell receptor (TCR) component and/or a signaling domain comprising an immunoreceptor tyrosine-based activation motif (ITAM), such as via an intracellular signaling domain or region of a CD3-zeta (CD3ζ) chain or a functional variant or signaling portion thereof.

In some embodiments, the recombinant receptor encoded from the modified TGFBR2 locus is a is a recombinant TCR. In some aspects, the recombinant TCR comprises two polypeptide chains, for example, a TCR alpha (TCRα) and a TCR beta (TCRβ) chain; or a TCR gamma (TCRγ) and a TCR delta (TCRδ) chain. In some aspects, the modified TGFBR2 locus encodes one or more chains of the recombinant TCR. In some embodiments, the modified TGFBR2 locus encodes a TCRα. In some embodiments, the modified TGFBR2 locus encodes a TCRβ. In some embodiments, the modified TGFBR2 locus encodes a TCRα and a TCRβ, optionally separated by a multicistronic element such as a 2A element.

### B. Encoded Recombinant Receptors

In some embodiments, the recombinant receptor encoded by the engineered cells, for example at the modified TGFBR2 locus as described herein, or the engineered cells generated according to the methods provided herein, include a chimeric antigen receptor (CAR) or a portion thereof, or a recombinant T cell receptor (TCR) or a portion thereof. Among the recombinant receptors are chimeric receptors, antigen receptors and receptors containing one or more component of chimeric receptors or antigen receptors. The recombinant receptors may include those containing ligand-binding domains or binding fragments thereof and intracellular signaling domains or regions. In some embodiments, the recombinant receptors encoded by the engineered cells include functional non-TCR antigen receptors, chimeric antigen receptors (CARs), chimeric autoantibody receptor (CAAR), recombinant T cell receptors (TCRs) and region(s), chain(s), domain(s) or component(s) of any of the foregoing. In some aspects, the recombinant receptor or a portion thereof is encoded by transgene sequences present in the polynucleotides provided herein, such as any template polynucleotides described in Section I.B.2 above. In some aspects, the transgene sequence encoding the recombinant receptor or a portion thereof contained in the polynucleotides, is integrated at the endogenous TGFBR2 locus of the engineered cell, to result in a modified TGFBR2 locus that encodes a recombinant receptor or a portion thereof, such as any recombinant receptor described herein, including one or more polypeptide chains of a multi-chain recombinant receptor.

In some embodiments, exemplary recombinant receptors expressed from the engineered cell include multi-chain receptors that contain two or more receptor polypeptides, which, in some cases, contain different components, domains or regions. In some aspects, the recombinant receptor contains two or more polypeptides that together comprise a functional recombinant receptor. In some aspects, the multi-chain receptor is a dual-chain receptor, comprising two polypeptides that together comprise a functional recombinant receptor. In some embodiments, the recombinant receptor is a TCR comprising two different receptor polypeptides, for example, a TCR alpha (TCRα) and a TCR beta (TCRβ) chain; or a TCR gamma (TCRγ) and a TCR delta (TCRδ) chain. In some embodiments, the recombinant receptor is a multi-chain receptor in which one or more of the polypeptides regulates, modifies or controls the expression, activity or function of another receptor polypeptide. In some aspects, multi-chain receptors allows spatial or temporal regulation or control of specificity, activity, antigen (or ligand) binding, function and/or expression of the receptor.

In some embodiments, the recombinant receptor, encoded in the genetically engineered cells provided herein, contains a transmembrane domain or a membrane association domain. In some aspects, the recombinant receptor also contains an extracellular region. In some aspects, the recombinant receptor also contains an intracellular region. In some embodiments, the recombinant receptor encoded in the genetically engineered cells provided herein contains various regions or domains such as one or more of extracellular region (e.g., containing one or more extracellular binding domain(s) and/or spacers), transmembrane domain and intracellular region (e.g., containing an intracellular signaling region and/or one or more costimulatory signaling domains). In some aspects, the encoded recombinant receptor further contains other domains, such as multimerization domains, linkers and/or regulatory elements.

In some embodiments, an exemplary encoded recombinant receptor comprises, in its N- to C-terminus order: a transmembrane domain (or a membrane association domain) and an intracellular region. In some embodiments, an exemplary encoded recombinant receptor comprises, in its N- to C-terminus order: an extracellular region, a transmembrane domain and an intracellular region. In some embodiments, the extracellular region is or comprises an extracellular binding domain and, in some aspects, the encoded recombinant receptor comprises, from its N to C terminus in order: an extracellular binding domain, a transmembrane domain and an intracellular region. In some cases, a spacer that separates or is positioned between the extracellular region, e.g. extracellular binding domain, and the transmembrane domain. In some embodiments, the encoded recombinant receptor comprises, from its N to C terminus in order: an extracellular binding domain, a spacer, a transmembrane domain and an intracellular region. In some embodiments, the intracellular signaling region present in a recombinant receptor contains an immunoreceptor tyrosine-based activation motif (ITAM) and/or one or more costimulatory signaling domains, such as one, two or three costimulatory signaling domains

In some embodiments, the recombinant receptor contains a multimerization domain, which in some aspects, is able to effect formation of a multi-chain polypeptide thereof. In some embodiments, an exemplary encoded recombinant receptor comprises, in its N- to C-terminus order: a transmembrane domain (or a membrane association domain), an intracellular multimerization domain, optionally one or more costimulatory signaling domain(s), and an intracellular signaling region. In some embodiments, an exemplary recombinant receptor polypeptide comprises, in its N- to C-terminus order: an extracellular multimerization domain, a transmembrane domain, optionally one or more costimulatory signaling domain(s), and an intracellular signaling region.

In some embodiments, the encoded recombinant receptor is a chimeric receptor, such as a CAR. An exemplary encoded CAR sequence comprises: an extracellular binding domain, a spacer, a transmembrane domain and an intracellular region comprising a primary signaling domain or region and one or more co-stimulatory signaling domain. In some embodiments, an exemplary encoded CAR sequence comprises: an extracellular binding domain, a spacer, a transmembrane domain and one or more costimulatory signaling domains and primary signaling domain or region.

In some embodiments, an exemplary encoded polypeptide, such as a polypeptide chain of a multi-chain CAR, sequence comprises: a transmembrane domain (or a membrane association domain), an intracellular multimerization domain, optionally one or more costimulatory signaling domain(s), and a primary signaling domain or region. In some embodiments, an exemplary encoded polypeptide, such as a polypeptide chain of a multi-chain CAR, sequence comprises: an extracellular multimerization domain, a transmembrane domain, optionally one or more costimulatory signaling domain(s), and a primary signaling domain or region.

In some embodiments, an exemplary encoded CAR sequence comprises, in order a sequence of nucleotides encoding an extracellular binding domain, optionally an scFv; a spacer, optionally comprising a sequence from a human immunoglobulin hinge, optionally from IgG1, IgG2 or IgG4 or a modified version thereof, optionally further comprising a C_{H}2 region and/or a C_{H}3 region; and a transmembrane domain, optionally from human CD28; a costimulatory signaling domain, optionally from human 4-1BB; and an intracellular signaling region, optionally a CD3ζ chain or a portion thereof. In some embodiments, the encoded intracellular region of the recombinant receptor comprises, from its N to C terminus in order: the one or more costimulatory signaling domain(s) and a primary signaling domain or region, such as containing a CD3zeta chain or a fragment thereof.

In some embodiments, the encoded recombinant receptor is a recombinant TCR and an exemplary encoded TCR includes, a TCRα chain or a TCRβ chain or both. In some embodiments, an exemplary encoded polypeptide, such as a polypeptide of a recombinant receptor, comprises all or a portion of a TCRα chain. In some embodiments, an exemplary encoded polypeptide, such as a polypeptide of a recombinant receptor, comprises all or a portion of a TCRβ chain. In some aspects, an exemplary encoded recombinant receptor is a recombinant TCR comprising a TCRα chain and a TCRβ chain.

1. Chimeric Antigen Receptors (CARs)

In some embodiments, the recombinant receptor encoded by the modified TGFBR2 locus is a chimeric antigen receptor (CAR). In some embodiments, the engineered cells, such as T cells, express a recombinant receptor such as a CAR, with specificity for a particular antigen (or marker or ligand), such as an antigen expressed on the surface of a particular cell type. In some aspects, at least a portion of any of the CARs described herein, including multi-chain or regulatable CAR, is encoded in the transgene sequences. In some aspects, the transgene sequences encoding the CARs described herein or a portion thereof, can be any described in Section I.B.2. In some aspects, upon integration of the transgene sequences via HDR, the resulting modified TGFBR2 locus contains nucleic acid sequences encoding a CAR, such as any CAR described herein, including multi-chain or regulatable CAR.

In some embodiments, the recombinant receptor, e.g., CAR, encoded by the modified TGFBR2 locus, contains one or more of extracellular region (e.g., containing one or more extracellular binding domain(s) and/or spacers), transmembrane domain and/or intracellular region (e.g., containing a primary signaling region or domain and/or one or more costimulatory signaling domains). In some aspects, the encoded recombinant receptor further contains other domains, such as multimerization domains. In some aspects, the modified TGFBR2 locus contains sequences encoding linkers and/or regulatory elements. In some embodiments, the encoded recombinant receptor comprises, from its N to C terminus in order: an extracellular binding domain, a transmembrane domain and an intracellular region, e.g., comprising a primary signaling region or domain or a portion thereof and/or a costimulatory signaling domain. In some embodiments, the encoded recombinant receptor comprises, from its N to C terminus in order: an extracellular binding domain, a spacer, a transmembrane domain and an intracellular region, e.g., comprising a primary signaling region or domain or a portion thereof and/or a costimulatory signaling domain.

a. Binding Domain

In some embodiments, the extracellular region of the encoded recombinant receptor comprises a binding domain. In some embodiments, the binding domain is an extracellular binding domain. In some embodiments, the binding domain is or comprises a polypeptide, a ligand, a receptor, a ligand-binding domain, a receptor-binding domain, an antigen, an epitope, an antibody, an antigen-binding domain, an epitope-binding domain, an antibody-binding domain, a tag-binding domain or a fragment of any of the foregoing. In some embodiments, the binding domain is a ligand- or antigen-binding domain.

In some aspects, the extracellular binding domain, such as a ligand- (e.g., antigen-) binding region or domain(s) and the intracellular region or domain(s) are linked or connected via one or more linkers and/or transmembrane domain(s). In some embodiments, the chimeric antigen receptor includes a transmembrane domain disposed between the extracellular region and the intracellular region.

In some embodiments, the antigen, e.g., an antigen that binds the binding domain of the recombinant receptor, is a polypeptide. In some embodiments, the antigen is a carbohydrate or other molecule. In some embodiments, the antigen is selectively expressed or overexpressed on cells of the disease, disorder or condition, e.g., the tumor or pathogenic cells, as compared to normal or non-targeted cells or tissues, e.g., in healthy cells or tissues. In some embodiments, the disease, disorder or condition is an infectious disease or disorder, an autoimmune disease, an inflammatory disease, or a tumor or a cancer. In some embodiments, the antigen is expressed on normal cells and/or is expressed on the engineered cells. In some aspects, the recombinant receptor, e.g., a CAR, includes one or more regions or domains selected from an extracellular ligand- (e.g., antigen-) binding or region or domains, e.g., any of the antibody or fragment described herein, and an intracellular region. In some embodiments, the ligand- (e.g., antigen-) binding region or domain is or includes an scFv or a single-domain V_{H} antibody and the intracellular region comprises an intracellular signaling region or domain comprising an immunoreceptor tyrosine-based activation motif (ITAM).

Exemplary encoded recombinant receptors, including CARs, include those described, for example, in International Pat. App. Pub. Nos. WO2000/14257, WO2013/126726, WO2012/129514, WO2014/031687, WO2013/166321, WO2013/071154, WO2013/123061, U.S. Pat. App. Pub. Nos. US2002131960, US2013287748, US20130149337, U.S. Pat. Nos. 6,451,995, 7,446,190, 8,252,592, 8,339,645, 8,398,282, 7,446,179, 6,410,319, 7,070,995, 7,265,209, 7,354,762, 7,446,191, 8,324,353, and 8,479,118, and European Pat. App. No. EP2537416, and/or those described by Sadelain et al., Cancer Discov. 2013 April; 3(4): 388-398; Davila et al. (2013) PLoS ONE 8(4): e61338; Turtle et al., Curr. Opin. Immunol., 2012 October; 24(5): 633-39; and Wu et al., Cancer, 2012 Mar. 18(2): 160-75. In some aspects, the antigen receptors include a CAR as described in U.S. Pat. No. 7,446,190, and those described in International Pat. App. Pub. No. Pub. No WO 2014/055668. Examples of the CARs include CARs as disclosed in any of the aforementioned references, such as WO2014/031687, U.S. Pat. Nos. 8,339,645, 7,446,179, US 2013/0149337, U.S. Pat. Nos. 7,446,190, 8,389,282, Kochenderfer et al., 2013, Nature Reviews Clinical Oncology, 10, 267-276 (2013); Wang et al. (2012) J. Immunother. 35(9): 689-701; and Brentjens et al., Sci Transl Med. 2013 5(177).

In some embodiments, the encoded recombinant receptor, e.g., antigen receptor contains an extracellular binding domain, such as an antigen- or ligand-binding domain that binds, e.g., specifically binds, to an antigen, a ligand and/or a marker. Among the antigen receptors are functional non-TCR antigen receptors, such as chimeric antigen receptors (CARs). In some embodiments, the antigen receptor is a CAR that contains an extracellular antigen-recognition domain that specifically binds to an antigen. In some embodiments, the CAR is constructed with a specificity for a particular antigen, marker or ligand, such as an antigen expressed in a particular cell type to be targeted by adoptive therapy, e.g., a cancer marker, and/or an antigen intended to induce a dampening response, such as an antigen expressed on a normal or non-diseased cell type. Thus, the CAR typically includes in its extracellular portion one or more ligand- (e.g., antigen-) binding molecules, such as one or more antigen-binding fragment, domain, or portion, or one or more antibody variable domains, and/or antibody molecules. In some embodiments, the CAR includes an antigen-binding portion or portions of an antibody molecule, such as a single-chain antibody fragment (scFv) derived from the variable heavy (V_{H}) and variable light (V_{L}) chains of a monoclonal antibody (mAb), or a single domain antibody (sdAb), such as sdFv, nanobody, V_{H}H and V_{NAR}. In some embodiments, an antigen-binding fragment comprises antibody variable regions joined by a flexible linker.

In some embodiments, the encoded CAR contains an antibody or an antigen-binding fragment (e.g. scFv) that specifically recognizes an antigen or ligand, such as an intact antigen, expressed on the surface of a cell. In some embodiments, the antigen or ligand, is a protein expressed on the surface of cells. In some embodiments, the antigen or ligand is a polypeptide. In some embodiments, it is a carbohydrate or other molecule. In some embodiments, the antigen or ligand is selectively expressed or overexpressed on cells of the disease or condition, e.g., the tumor or pathogenic cells, as compared to normal or non-targeted cells or tissues. In other embodiments, the antigen is expressed on normal cells and/or is expressed on the engineered cells.

In some embodiments, among the antigens targeted by the recombinant receptors are those expressed in the context of a disease, condition, or cell type to be targeted via the adoptive cell therapy. Among the diseases and conditions are proliferative, neoplastic, and malignant diseases and disorders, including cancers and tumors, including hematologic malignancy, cancers of the immune system, such as lymphomas, leukemias, and/or myelomas, such as B, T, and myeloid leukemias, lymphomas, and multiple myelomas.

In some embodiments, the antigen or ligand is a tumor antigen or cancer marker. In some embodiments, the antigen associated with the disease or disorder is or includes αvβ6 integrin (avb6 integrin), B cell maturation antigen (BCMA), B7-H3, B7-H6, carbonic anhydrase 9 (CA9, also known as CAIX or G250), a cancer-testis antigen, cancer/testis antigen 1B (CTAG, also known as NY-ESO-1 and LAGE-2), carcinoembryonic antigen (CEA), a cyclin, cyclin A2, C—C Motif Chemokine Ligand 1 (CCL-1), CD19, CD20, CD22, CD23, CD24, CD30, CD33, CD38, CD44, CD44v6, CD44v7/8, CD123, CD133, CD138, CD171, chondroitin sulfate proteoglycan 4 (CSPG4), epidermal growth factor protein (EGFR), type III epidermal growth factor receptor mutation (EGFR vIII), epithelial glycoprotein 2 (EPG-2), epithelial glycoprotein 40 (EPG-40), ephrinB2, ephrin receptor A2 (EPHa2), estrogen receptor, Fc receptor like 5 (FCRL5; also known as Fc receptor homolog 5 or FCRH5), fetal acetylcholine receptor (fetal AchR), a folate binding protein (FBP), folate receptor alpha, ganglioside GD2, O-acetylated GD2 (OGD2), ganglioside GD3, glycoprotein 100 (gp100), glypican-3 (GPC3), G protein-coupled receptor class C group 5 member D (GPRC5D), Her2/neu (receptor tyrosine kinase erb-B2), Her3 (erb-B3), Her4 (erb-B4), erbB dimers, Human high molecular weight-melanoma-associated antigen (HMW-MAA), hepatitis B surface antigen, Human leukocyte antigen A1 (HLA-A1), Human leukocyte antigen A2 (HLA-A2), IL-22 receptor alpha (IL-22Rα), IL-13 receptor alpha 2 (IL-13Rα2), kinase insert domain receptor (kdr), kappa light chain, L1 cell adhesion molecule (L1-CAM), CE7 epitope of Li-CAM, Leucine Rich Repeat Containing 8 Family Member A (LRRC8A), Lewis Y, Melanoma-associated antigen (MAGE)-A1, MAGE-A3, MAGE-A6, MAGE-A10, mesothelin (MSLN), c-Met, murine cytomegalovirus (CMV), mucin 1 (MUC1), MUC16, natural killer group 2 member D (NKG2D) ligands, melan A (MART-1), neural cell adhesion molecule (NCAM), oncofetal antigen, Preferentially expressed antigen of melanoma (PRAME), progesterone receptor, a prostate specific antigen, prostate stem cell antigen (PSCA), prostate specific membrane antigen (PSMA), Receptor Tyrosine Kinase Like Orphan Receptor 1 (ROR1), survivin, Trophoblast glycoprotein (TPBG also known as 5T4), tumor-associated glycoprotein 72 (TAG72), Tyrosinase related protein 1 (TRP1, also known as TYRP1 or gp75), Tyrosinase related protein 2 (TRP2, also known as dopachrome tautomerase, dopachrome delta-isomerase or DCT), vascular endothelial growth factor receptor (VEGFR), vascular endothelial growth factor receptor 2 (VEGFR2), Wilms Tumor 1 (WT-1), a pathogen-specific or pathogen-expressed antigen, or an antigen associated with a universal tag, and/or biotinylated molecules, and/or molecules expressed by HIV, HCV, HBV or other pathogens. Antigens targeted by the receptors in some embodiments include antigens associated with a B cell malignancy, such as any of a number of known B cell marker. In some embodiments, the antigen is or includes CD20, CD19, CD22, ROR1, CD45, CD21, CD5, CD33, Igkappa, Iglambda, CD79a, CD79b or CD30.

In some embodiments, the antigen is or includes a pathogen-specific or pathogen-expressed antigen. In some embodiments, the antigen is a viral antigen (such as a viral antigen from HIV, HCV, HBV, etc.), bacterial antigens, and/or parasitic antigens.

In some embodiments, the antibody or an antigen-binding fragment (e.g. scFv or V_{H} domain) specifically recognizes an antigen, such as CD19. In some embodiments, the antibody or antigen-binding fragment is derived from, or is a variant of, antibodies or antigen-binding fragment that specifically binds to CD19.

In some embodiments, the scFv is derived from FMC63. FMC63 generally refers to a mouse monoclonal IgG1 antibody raised against Nalm-1 and -16 cells expressing CD19 of human origin (Ling, N. R., et al. (1987). Leucocyte typing III. 302). In some embodiments, the FMC63 antibody comprises a CDR-H1 and a CDR-H2 set forth in SEQ ID NOS: 38 and 39, respectively, and a CDR-H3 set forth in SEQ ID NO: 40 or 54; and a CDR-L1 set forth in SEQ ID NO: 35 and a CDR-L2 set forth in SEQ ID NO: 36 or 55 and a CDR-L3 set forth in SEQ ID NO: 37 or 56. In some embodiments, the FMC63 antibody comprises a heavy chain variable region (V_{H}) comprising the amino acid sequence of SEQ ID NO: 41 and a light chain variable region (V_{L}) comprising the amino acid sequence of SEQ ID NO: 42.

In some embodiments, the scFv comprises a variable light chain containing a CDR-L1 sequence of SEQ ID NO:35, a CDR-L2 sequence of SEQ ID NO:36, and a CDR-L3 sequence of SEQ ID NO:37 and/or a variable heavy chain containing a CDR-H1 sequence of SEQ ID NO:38, a CDR-H2 sequence of SEQ ID NO:39, and a CDR-H3 sequence of SEQ ID NO:40. In some embodiments, the scFv comprises a variable heavy chain region set forth in SEQ ID NO:41 and a variable light chain region set forth in SEQ ID NO:42. In some embodiments, the variable heavy and variable light chains are connected by a linker. In some embodiments, the linker is set forth in SEQ ID NO:58. In some embodiments, the scFv comprises, in order, a V_{H}, a linker, and a V_{L}. In some embodiments, the scFv comprises, in order, a V_{L}, a linker, and a V_{H}. In some embodiments, the scFv is encoded by a sequence of nucleotides set forth in SEQ ID NO:57 or a sequence that exhibits at least 85%, 86%, 87%, 88%, 89%, 90%, 91%, 92%, 93%, 94%, 95%, 96%, 97%, 98%, or 99% sequence identity to SEQ ID NO:57. In some embodiments, the scFv comprises the sequence of amino acids set forth in SEQ ID NO:43 or a sequence that exhibits at least 85%, 86%, 87%, 88%, 89%, 90%, 91%, 92%, 93%, 94%, 95%, 96%, 97%, 98%, or 99% sequence identity to SEQ ID NO:43.

In some embodiments the scFv is derived from SJ25C1. SJ25C1 is a mouse monoclonal IgG1 antibody raised against Nalm-1 and -16 cells expressing CD19 of human origin (Ling, N. R., et al. (1987). Leucocyte typing III. 302). In some embodiments, the SJ25C1 antibody comprises a CDR-H1, a CDR-H2 and a CDR-H3 sequence set forth in SEQ ID NOS: 47-49, respectively, and a CDR-L1, a CDR-L2 and a CDR-L3 sequence set forth in SEQ ID NOS: 44-46, respectively. In some embodiments, the SJ25C1 antibody comprises a heavy chain variable region (V_{H}) comprising the amino acid sequence of SEQ ID NO: 50 and a light chain variable region (V_{L}) comprising the amino acid sequence of SEQ ID NO: 51.

In some embodiments, the scFv comprises a variable light chain containing a CDR-L1 sequence of SEQ ID NO:44, a CDR-L2 sequence of SEQ ID NO: 45, and a CDR-L3 sequence of SEQ ID NO:46 and/or a variable heavy chain containing a CDR-H1 sequence of SEQ ID NO:47, a CDR-H2 sequence of SEQ ID NO:48, and a CDR-H3 sequence of SEQ ID NO:49. In some embodiments, the scFv comprises a variable heavy chain region set forth in SEQ ID NO:50 and a variable light chain region set forth in SEQ ID NO:51. In some embodiments, the variable heavy and variable light chain are connected by a linker. In some embodiments, the linker is set forth in SEQ ID NO:52. In some embodiments, the scFv comprises, in order, a V_{H}, a linker, and a V_{L}. In some embodiments, the scFv comprises, in order, a V_{L}, a linker, and a V_{H}. In some embodiments, the scFv comprises the sequence of amino acids set forth in SEQ ID NO:53 or a sequence that exhibits at least 85%, 86%, 87%, 88%, 89%, 90%, 91%, 92%, 93%, 94%, 95%, 96%, 97%, 98%, or 99% sequence identity to SEQ ID NO:53.

In some embodiments, the antigen is CD20. In some embodiments, the scFv contains a V_{H} and a V_{L} derived from an antibody or an antibody fragment specific to CD20. In some embodiments, the antibody or antibody fragment that binds CD20 is an antibody that is or is derived from Rituximab, such as is Rituximab scFv.

In some embodiments, the antigen is CD22. In some embodiments, the scFv contains a V_{H} and a V_{L} derived from an antibody or an antibody fragment specific to CD22. In some embodiments, the antibody or antibody fragment that binds CD22 is an antibody that is or is derived from m971, such as is m971 scFv.

In some embodiments, the antigen is BCMA. In some embodiments, the scFv contains a V_{H} and a V_{L} derived from an antibody or an antibody fragment specific to BCMA. In some embodiments, the antibody or antibody fragment that binds BCMA is or contains a V_{H} and a V_{L} from an antibody or antibody fragment set forth in International Patent Applications, Publication Number WO 2016/090327 and WO 2016/090320.

In some embodiments, the antigen is GPRC5D. In some embodiments, the scFv contains a V_{H} and a V_{L} derived from an antibody or an antibody fragment specific to GPRC5D. In some embodiments, the antibody or antibody fragment that binds GPRC5D is or contains a V_{H} and a V_{L} from an antibody or antibody fragment set forth in International Patent Applications, Publication Number WO 2016/090329 and WO 2016/090312.

In some aspects, the encoded CAR contains a ligand- (e.g., antigen-) binding domain that binds or recognizes, e.g., specifically binds, a universal tag or a universal epitope. In some aspects, the binding domain can bind a molecule, a tag, a polypeptide and/or an epitope that can be linked to a different binding molecule (e.g., antibody or antigen-binding fragment) that recognizes an antigen associated with a disease or disorder. Exemplary tag or epitope includes a dye (e.g., fluorescein isothiocyanate) or a biotin. In some aspects, a binding molecule (e.g., antibody or antigen-binding fragment) linked to a tag, that recognizes the antigen associated with a disease or disorder, e.g., tumor antigen, with an engineered cell expressing a CAR specific for the tag, to effect cytotoxicity or other effector function of the engineered cell. In some aspects, the specificity of the CAR to the antigen associated with a disease or disorder is provided by the tagged binding molecule (e.g., antibody), and different tagged binding molecule can be used to target different antigens. Exemplary CARs specific for a universal tag or a universal epitope include those described, e.g., in U.S. Pat. No. 9,233,125, WO 2016/030414, Urbanska et al., (2012) Cancer Res 72: 1844-1852, and Tamada et al., (2012) Clin Cancer Res 18:6436-6445.

In some embodiments, the encoded CAR contains a TCR-like antibody, such as an antibody or an antigen-binding fragment (e.g. scFv) that specifically recognizes an intracellular antigen, such as a tumor-associated antigen, presented on the cell surface as a major histocompatibility complex (MHC)-peptide complex. In some embodiments, an antibody or antigen-binding portion thereof that recognizes an MHC-peptide complex can be expressed on cells as part of a recombinant receptor, such as an antigen receptor. Among the antigen receptors are functional non-T cell receptor (TCR) antigen receptors, such as chimeric antigen receptors (CARs). In some embodiments, a CAR containing an antibody or antigen-binding fragment that exhibits TCR-like specificity directed against peptide-MHC complexes also may be referred to as a TCR-like CAR. In some embodiments, the CAR is a TCR-like CAR and the antigen is a processed peptide antigen, such as a peptide antigen of an intracellular protein, which, like a TCR, is recognized on the cell surface in the context of an MHC molecule. In some embodiments, the extracellular antigen-binding domain specific for an MHC-peptide complex of a TCR-like CAR is linked to one or more intracellular signaling components, in some aspects via linkers and/or transmembrane domain(s). In some embodiments, such molecules can typically mimic or approximate a signal through a natural antigen receptor, such as a TCR, and, optionally, a signal through such a receptor in combination with a costimulatory receptor.

In some embodiments, Major histocompatibility complex (MHC) includes a protein, generally a glycoprotein, that contains a polymorphic peptide binding site or binding groove that can, in some cases, complex with peptide antigens of polypeptides, including peptide antigens processed by the cell machinery. In some cases, MHC molecules can be displayed or expressed on the cell surface, including as a complex with peptide, i.e. MHC-peptide complex, for presentation of an antigen in a conformation recognizable by an antigen receptor on T cells, such as a TCRs or TCR-like antibody. Generally, MHC class I molecules are heterodimers having a membrane spanning α chain, in some cases with three α domains, and a non-covalently associated β2 microglobulin. Generally, MHC class II molecules are composed of two transmembrane glycoproteins, α and β, both of which typically span the membrane. An MHC molecule can include an effective portion of an MHC that contains an antigen binding site or sites for binding a peptide and the sequences necessary for recognition by the appropriate antigen receptor. In some embodiments, MHC class I molecules deliver peptides originating in the cytosol to the cell surface, where a MHC-peptide complex is recognized by T cells, such as generally CD8⁺ T cells, but in some cases CD4⁺ T cells. In some embodiments, MHC class II molecules deliver peptides originating in the vesicular system to the cell surface, where they are typically recognized by CD4⁺ T cells. Generally, MHC molecules are encoded by a group of linked loci, which are collectively termed H-2 in the mouse and human leukocyte antigen (HLA) in humans. Hence, typically human MHC can also be referred to as human leukocyte antigen (HLA).

The term “MHC-peptide complex” or “peptide-MHC complex” or variations thereof, refers to a complex or association of a peptide antigen and an MHC molecule, such as, generally, by non-covalent interactions of the peptide in the binding groove or cleft of the MHC molecule. In some embodiments, the MHC-peptide complex is present or displayed on the surface of cells. In some embodiments, the MHC-peptide complex can be specifically recognized by an antigen receptor, such as a TCR, TCR-like CAR or antigen-binding portions thereof.

In some embodiments, a peptide, such as a peptide antigen or epitope, of a polypeptide can associate with an MHC molecule, such as for recognition by an antigen receptor. Generally, the peptide is derived from or based on a fragment of a longer biological molecule, such as a polypeptide or protein. In some embodiments, the peptide typically is about 8 to about 24 amino acids in length. In some embodiments, a peptide has a length of from or from about 9 to 22 amino acids for recognition in the MHC Class II complex. In some embodiments, a peptide has a length of from or from about 8 to 13 amino acids for recognition in the MHC Class I complex. In some embodiments, upon recognition of the peptide in the context of an MHC molecule, such as MHC-peptide complex, the antigen receptor, such as TCR or TCR-like CAR, produces or triggers an activation signal to the T cell that induces a T cell response, such as T cell proliferation, cytokine production, a cytotoxic T cell response or other response.

In some embodiments, a TCR-like antibody or antigen-binding portion, are known or can be produced by known methods (see e.g., US Pat. App. Pub. Nos. US 2002/0150914; US 2003/0223994; US 2004/0191260; US 2006/0034850; US 2007/00992530; US20090226474; US20090304679; and International App. Pub. No. WO 03/068201).

In some embodiments, an antibody or antigen-binding portion thereof that specifically binds to a MHC-peptide complex, can be produced by immunizing a host with an effective amount of an immunogen containing a specific MHC-peptide complex. In some cases, the peptide of the MHC-peptide complex is an epitope of antigen capable of binding to the MHC, such as a tumor antigen, for example a universal tumor antigen, myeloma antigen or other antigen as described herein. In some embodiments, an effective amount of the immunogen is then administered to a host for eliciting an immune response, wherein the immunogen retains a three-dimensional form thereof for a period of time sufficient to elicit an immune response against the three-dimensional presentation of the peptide in the binding groove of the MHC molecule. Serum collected from the host is then assayed to determine if desired antibodies that recognize a three-dimensional presentation of the peptide in the binding groove of the MHC molecule is being produced. In some embodiments, the produced antibodies can be assessed to confirm that the antibody can differentiate the MHC-peptide complex from the MHC molecule alone, the peptide of interest alone, and a complex of MHC and irrelevant peptide. The desired antibodies can then be isolated.

In some embodiments, an antibody or antigen-binding portion thereof that specifically binds to an MHC-peptide complex can be produced by employing antibody library display methods, such as phage antibody libraries. In some embodiments, phage display libraries of mutant Fab, scFv or other antibody forms can be generated, for example, in which members of the library are mutated at one or more residues of a CDR or CDRs. See e.g. US Pat. App. Pub. No. US20020150914, US20140294841; and Cohen C J. et al. (2003) J Mol. Recogn. 16:324-332.

The term “antibody” herein is used in the broadest sense and includes polyclonal and monoclonal antibodies, including intact antibodies and functional (antigen-binding) antibody fragments, including fragment antigen binding (Fab) fragments, F(ab′)₂ fragments, Fab′ fragments, Fv fragments, recombinant IgG (rIgG) fragments, variable heavy chain (V_{H}) regions capable of specifically binding the antigen, single chain antibody fragments, including single chain variable fragments (scFv), and single domain antibodies (e.g., sdAb, sdFv, nanobody, V_{H}H or V_{NAR}) or fragments. The term encompasses genetically engineered and/or otherwise modified forms of immunoglobulins, such as intrabodies, peptibodies, chimeric antibodies, fully human antibodies, humanized antibodies, and heteroconjugate antibodies, multispecific, e.g., bispecific, antibodies, diabodies, triabodies, and tetrabodies, tandem di-scFv, tandem tri-scFv. Unless otherwise stated, the term “antibody” should be understood to encompass functional antibody fragments thereof. The term also encompasses intact or full-length antibodies, including antibodies of any class or sub-class, including IgG and sub-classes thereof, IgM, IgE, IgA, and IgD. In some aspects, the CAR is a bispecific CAR, e.g., containing two antigen-binding domains with different specificities.

In some embodiments, the antigen-binding proteins, antibodies and antigen binding fragments thereof specifically recognize an antigen of a full-length antibody. In some embodiments, the heavy and light chains of an antibody can be full-length or can be an antigen-binding portion (a Fab, F(ab′)2, Fv or a single chain Fv fragment (scFv)). In other embodiments, the antibody heavy chain constant region is chosen from, e.g., IgG1, IgG2, IgG3, IgG4, IgM, IgA1, IgA2, IgD, and IgE, particularly chosen from, e.g., IgG1, IgG2, IgG3, and IgG4, more particularly, IgG1 (e.g., human IgG1). In some embodiments, the antibody light chain constant region is chosen from, e.g., kappa or lambda, particularly kappa.

Among the binding domains of the encoded recombinant receptors are antibody fragments. An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂; diabodies; linear antibodies; variable heavy chain (V_{H}) regions, single-chain antibody molecules such as scFvs and single-domain V_{H} single antibodies; and multispecific antibodies formed from antibody fragments. In particular embodiments, the antibodies are single-chain antibody fragments comprising a variable heavy chain region and/or a variable light chain region, such as scFvs.

The term “variable region” or “variable domain” refers to the domain of an antibody heavy or light chain that is involved in binding the antibody to antigen. The variable domains of the heavy chain and light chain (V_{H} and V_{L}, respectively) of a native antibody generally have similar structures, with each domain comprising four conserved framework regions (FRs) and three CDRs. (See, e.g., Kindt et al. Kuby Immunology, 6th ed., W.H. Freeman and Co., page 91 (2007). A single V_{H} or V_{L} domain may be sufficient to confer antigen-binding specificity. Furthermore, antibodies that bind a particular antigen may be isolated using a V_{H} or V_{L} domain from an antibody that binds the antigen to screen a library of complementary V_{L} or V_{H} domains, respectively. See, e.g., Portolano et al., J. Immunol. 150:880-887 (1993); Clarkson et al., Nature 352:624-628 (1991).

Single-domain antibodies (sdAb) are antibody fragments comprising all or a portion of the heavy chain variable domain or all or a portion of the light chain variable domain of an antibody. In certain embodiments, a single-domain antibody is a human single-domain antibody. In some embodiments, the CAR comprises an antibody heavy chain domain that specifically binds the antigen, such as a cancer marker or cell surface antigen of a cell or disease to be targeted, such as a tumor cell or a cancer cell, such as any of the target antigens described herein or known. Exemplary single-domain antibodies include sdFv, nanobody, V_{H}H or V_{NAR}.

Antibody fragments can be made by various techniques, including but not limited to proteolytic digestion of an intact antibody as well as production by recombinant host cells. In some embodiments, the antibodies are recombinantly produced fragments, such as fragments comprising arrangements that do not occur naturally, such as those with two or more antibody regions or chains joined by synthetic linkers, e.g., peptide linkers, and/or that are may not be produced by enzyme digestion of a naturally-occurring intact antibody. In some embodiments, the antibody fragments are scFvs.

A “humanized” antibody is an antibody in which all or substantially all CDR amino acid residues are derived from non-human CDRs and all or substantially all FR amino acid residues are derived from human FRs. A humanized antibody optionally may include at least a portion of an antibody constant region derived from a human antibody. A “humanized form” of a non-human antibody, refers to a variant of the non-human antibody that has undergone humanization, typically to reduce immunogenicity to humans, while retaining the specificity and affinity of the parental non-human antibody. In some embodiments, some FR residues in a humanized antibody are substituted with corresponding residues from a non-human antibody (e.g., the antibody from which the CDR residues are derived), e.g., to restore or improve antibody specificity or affinity.

Thus, in some embodiments, the encoded chimeric antigen receptor, including TCR-like CARs, includes an extracellular portion containing an antibody or antibody fragment. In some embodiments, the antibody or fragment includes an scFv. In some aspects, the antibody or antigen-binding fragment can be obtained by screening a plurality, such as a library, of antigen-binding fragments or molecules, such as by screening an scFv library for binding to a specific antigen or ligand.

In some embodiments, the encoded CAR is a multi-specific CAR, e.g., contains a plurality of ligand- (e.g., antigen-) binding domains that can bind and/or recognize, e.g., specifically bind, a plurality of different antigens. In some aspects, the encoded CAR is a bispecific CAR, for example, targeting two antigens, such as by containing two antigen-binding domains with different specificities. In some embodiments, the CAR contains a bispecific binding domain, e.g., a bispecific antibody or fragment thereof, containing at least one antigen-binding domain binding to different surface antigens on a target cell, e.g., selected from any of the listed antigens as described herein, e.g. CD19 and CD22 or CD19 and CD20. In some embodiments, binding of the bispecific binding domain to each of its epitope or antigen can result in stimulation of function, activity and/or responses of the T cell, e.g., cytotoxic activity and subsequent lysis of the target cell. Among such exemplary bispecific binding domain can include tandem scFv molecules, in some cases fused to each other via, e.g. a flexible linker; diabodies and derivatives thereof, including tandem diabodies (Holliger et al, Prot Eng 9, 299-305 (1996); Kipriyanov et al, J Mol Biol 293, 41-66 (1999)); dual affinity retargeting (DART) molecules that can include the diabody format with a C-terminal disulfide bridge; bispecific T cell engager (BiTE) molecules, which contain tandem scFv molecules fused by a flexible linker (see e.g. Nagorsen and Bauerle, Exp Cell Res 317, 1255-1260 (2011); or triomabs that include whole hybrid mouse/rat IgG molecules (Seimetz et al, Cancer Treat Rev 36, 458-467 (2010). Any of such binding domains can be contained in any of the CARs described herein.

b. Spacer and Transmembrane Domain

In some aspects, the encoded recombinant receptor, e.g., a chimeric antigen receptor (CAR), includes an extracellular portion containing one or more ligand- (e.g., antigen-) binding domains, such as an antibody or fragment thereof, and one or more intracellular signaling region or domain (also interchangeably called a cytoplasmic signaling domain or region). In some aspects, the recombinant receptor, e.g., CAR, further includes a spacer and/or a transmembrane domain or portion. In some aspects, the spacer and/or transmembrane domain can link the extracellular portion containing the ligand- (e.g., antigen-) binding domain and the intracellular signaling region(s) or domain(s).

In some embodiments, the encoded recombinant receptor such as the CAR further includes a spacer, which may be or include at least a portion of an immunoglobulin constant region or variant or modified version thereof, such as a hinge region, e.g., an IgG4 hinge region, and/or a C_{H}1/C_{L} and/or Fc region. In some embodiments, the recombinant receptor further comprises a spacer and/or a hinge region. In some embodiments, the constant region or portion is of a human IgG, such as IgG4, IgG2 or IgG1. In some aspects, the portion of the constant region serves as a spacer region between the antigen-recognition component, e.g., scFv, and transmembrane domain. The spacer can be of a length that provides for increased responsiveness of the cell following antigen binding, as compared to in the absence of the spacer. In some examples, the spacer is at or about 12 amino acids in length or is no more than 12 amino acids in length. Exemplary spacers include those having at least about 10 to 229 amino acids, about 10 to 200 amino acids, about 10 to 175 amino acids, about 10 to 150 amino acids, about 10 to 125 amino acids, about 10 to 100 amino acids, about 10 to 75 amino acids, about 10 to 50 amino acids, about 10 to 40 amino acids, about 10 to 30 amino acids, about 10 to 20 amino acids, or about 10 to 15 amino acids, and including any integer between the endpoints of any of the listed ranges. In some embodiments, a spacer region has about 12 amino acids or less, about 119 amino acids or less, or about 229 amino acids or less. In some embodiments, the spacer is less than 250 amino acids in length, less than 200 amino acids in length, less than 150 amino acids in length, less than 100 amino acids in length, less than 75 amino acids in length, less than 50 amino acids in length, less than 25 amino acids in length, less than 20 amino acids in length, less than 15 amino acids in length, less than 12 amino acids in length, or less than 10 amino acids in length. In some embodiments, the spacer is from or from about 10 to 250 amino acids in length, 10 to 150 amino acids in length, 10 to 100 amino acids in length, 10 to 50 amino acids in length, 10 to 25 amino acids in length, 10 to 15 amino acids in length, 15 to 250 amino acids in length, 15 to 150 amino acids in length, 15 to 100 amino acids in length, 15 to 50 amino acids in length, 15 to 25 amino acids in length, 25 to 250 amino acids in length, 25 to 100 amino acids in length, 25 to 50 amino acids in length, 50 to 250 amino acids in length, 50 to 150 amino acids in length, 50 to 100 amino acids in length, 100 to 250 amino acids in length, 100 to 150 amino acids in length, or 150 to 250 amino acids in length. Exemplary spacers include IgG4 hinge alone, IgG4 hinge linked to C_{H}2 and C_{H}3 domains, or IgG4 hinge linked to the C_{H}3 domain. Exemplary spacers include, but are not limited to, those described in Hudecek et al. (2013) Clin. Cancer Res., 19:3153, Hudecek et al. (2015) Cancer Immunol Res. 3(2): 125-135 or International Pat. App. Pub. No. WO2014031687.

In some embodiments, the spacer can be derived all or in part from IgG4 and/or IgG2. In some embodiments, the spacer can be a chimeric polypeptide containing one or more of a hinge, C_{H}2 and/or C_{H}3 sequence(s) derived from IgG4, IgG2, and/or IgG2 and IgG4. In some embodiments, the spacer can contain mutations, such as one or more single amino acid mutations in one or more domains. In some examples, the amino acid modification is a substitution of a proline (P) for a serine (S) in the hinge region of an IgG4. In some embodiments, the amino acid modification is a substitution of a glutamine (Q) for an asparagine (N) to reduce glycosylation heterogeneity, such as an N to Q substitution at a position corresponding to position 177 in the C_{H}2 region of the IgG4 heavy chain constant region sequence set forth in SEQ ID NO: 184 (Uniprot Accession No. P01861; position corresponding to position 297 by EU numbering and position 79 of the hinge-C_{H}2-C_{H}3 spacer sequence set forth in SEQ ID NO:4) or an N to Q substitution at a position corresponding to position 176 in the C_{H}2 region of the IgG2 heavy chain constant region sequence set forth in SEQ ID NO: 183 (Uniprot Accession No. P01859; position corresponding to position 297 by EU numbering).

In some aspects, the spacer contains only a hinge region of an IgG, such as only a hinge of IgG4, IgG2 or IgG1, such as the hinge only spacer set forth in SEQ ID NO:1, and is encoded by the sequence set forth in SEQ ID NO: 2. In other embodiments, the spacer is an Ig hinge, e.g., and IgG4 hinge, linked to a C_{H}2 and/or C_{H}3 domains. In some embodiments, the spacer is an Ig hinge, e.g., an IgG4 hinge, linked to C_{H}2 and C_{H}3 domains, such as set forth in SEQ ID NO:3. In some embodiments, the spacer is an Ig hinge, e.g., an IgG4 hinge, linked to a C_{H}3 domain only, such as set forth in SEQ ID NO:4. In some embodiments, the spacer is or comprises a glycine-serine rich sequence or other flexible linker such as known flexible linkers. In some embodiments, the constant region or portion is of IgD. In some embodiments, the spacer has the sequence set forth in SEQ ID NO: 5. In some embodiments, the spacer has a sequence of amino acids that exhibits at least or at least about 85%, 86%, 87%, 88%, 89%, 90%, 91%, 92%, 93%, 94%, 95%, 96%, 97%, 98%, 99% or more sequence identity to any of SEQ ID NOS: 1, 3, 4 and 5.

In some aspects, the spacer is a polypeptide spacer such as one or more selected from: (a) comprises or consists of all or a portion of an immunoglobulin hinge or a modified version thereof or comprises about 15 amino acids or less, and does not comprise a CD28 extracellular region or a CD8 extracellular region, (b) comprises or consists of all or a portion of an immunoglobulin hinge, optionally an IgG4 hinge, or a modified version thereof and/or comprises about 15 amino acids or less, and does not comprise a CD28 extracellular region or a CD8 extracellular region, or (c) is at or about 12 amino acids in length and/or comprises or consists of all or a portion of an immunoglobulin hinge, optionally an IgG4, or a modified version thereof; or (d) consists or comprises the sequence of amino acids set forth in SEQ ID NOS: 1, 3-5 or 27-34, or a variant of any of the foregoing having at least 85%, 86%, 87%, 88%, 89%, 90%, 91%, 92%, 93%, 94%, 95%, 96%, 97%, 98%, 99% or more sequence identity thereto, or (e) comprises or consists of the formula X₁PPX₂P, where X₁ is glycine, cysteine or arginine and X₂ is cysteine or threonine.

Exemplary spacers include those containing portion(s) of an immunoglobulin constant region such as those containing an Ig hinge, such as an IgG hinge domain. In some aspects, the spacer includes an IgG hinge alone, an IgG hinge linked to one or more of a C_{H}2 and C_{H}3 domain, or IgG hinge linked to the C_{H}3 domain. In some embodiments, the IgG hinge, C_{H}2 and/or C_{H}3 can be derived all or in part from IgG4 or IgG2. In some embodiments, the spacer can be a chimeric polypeptide containing one or more of a hinge, C_{H}2 and/or C_{H}3 sequence(s) derived from IgG4, IgG2, and/or IgG2 and IgG4. In some embodiments, the hinge region comprises all or a portion of an IgG4 hinge region and/or of an IgG2 hinge region, wherein the IgG4 hinge region is optionally a human IgG4 hinge region and the IgG2 hinge region is optionally a human IgG2 hinge region; the C_{H}2 region comprises all or a portion of an IgG4 C_{H}2 region and/or of an IgG2 C_{H}2 region, wherein the IgG4 C_{H}2 region is optionally a human IgG4 C_{H}2 region and the IgG2 C_{H}2 region is optionally a human IgG2 C_{H}2 region; and/or the C_{H}3 region comprises all or a portion of an IgG4 C_{H}3 region and/or of an IgG2 C_{H}3 region, wherein the IgG4 C_{H}3 region is optionally a human IgG4 C_{H}3 region and the IgG2 C_{H}3 region is optionally a human IgG2 C_{H}3 region. In some embodiments, the hinge, C_{H}2 and C_{H}3 comprises all or a portion of each of a hinge region, C_{H}2 and C_{H}3 from IgG4. In some embodiments, the hinge region is chimeric and comprises a hinge region from human IgG4 and human IgG2; the C_{H}2 region is chimeric and comprises a C_{H}2 region from human IgG4 and human IgG2; and/or the C_{H}3 region is chimeric and comprises a C_{H}3 region from human IgG4 and human IgG2. In some embodiments, the spacer comprises an IgG4/2 chimeric hinge or a modified IgG4 hinge comprising at least one amino acid replacement compared to human IgG4 hinge region; an human IgG2/4 chimeric C_{H}2 region; and a human IgG4 C_{H}3 region.

In some embodiments, the spacer can be derived all or in part from IgG4 and/or IgG2 and can contain mutations, such as one or more single amino acid mutations in one or more domains. In some examples, the amino acid modification is a substitution of a proline (P) for a serine (S) in the hinge region of an IgG4. In some embodiments, the amino acid modification is a substitution of a glutamine (Q) for an asparagine (N) to reduce glycosylation heterogeneity, such as an N177Q mutation at position 177, in the C_{H}2 region, of the full-length IgG4 Fc sequence set forth in SEQ ID NO: 184 or an N176Q. at position 176, in the C_{H}2 region, of the full-length IgG2 Fc sequence set forth in SEQ ID NO: 183. In some embodiments, the spacer is or comprises an IgG4/2 chimeric hinge or a modified IgG4 hinge; an IgG2/4 chimeric C_{H}2 region; and an IgG4 C_{H}3 region and optionally is about 228 amino acids in length; or a spacer set forth in SEQ ID NO: 187. In some embodiments, the ligand- (e.g., antigen-) binding or recognition domain of the CAR is linked to an intracellular region, e.g., containing one or more intracellular signaling components, such as an intracellular signaling region or domain, and/or signaling components that mimic activation through an antigen receptor complex, such as a TCR complex, and/or signal via another cell surface receptor. Thus, in some embodiments, the extracellular region, e.g., containing a binding domain such as an antigen binding component (e.g., antibody), is linked to one or more transmembrane and intracellular region(s) or domain(s). In some embodiments, the transmembrane domain is fused to the extracellular region. In some embodiments, a transmembrane domain that naturally is associated with one of the domains in the receptor, e.g., CAR, is used. In some instances, the transmembrane domain is selected or modified by amino acid substitution to avoid binding of such domains to the transmembrane domains of the same or different surface membrane proteins to minimize interactions with other members of the receptor complex.

The transmembrane domain in some embodiments is derived either from a natural or from a synthetic source. Where the source is natural, the domain in some aspects is derived from any membrane-bound or transmembrane protein. Transmembrane regions include those derived from (i.e., comprise at least the transmembrane region(s) of) the alpha, beta or zeta chain of the T-cell receptor, CD28, CD3 epsilon, CD45, CD4, CD5, CD8, CD9, CD16, CD22, CD33, CD37, CD64, CD80, CD86, CD134, CD137 (4-1BB), or CD154. Alternatively the transmembrane domain in some embodiments is synthetic. In some aspects, the synthetic transmembrane domain comprises predominantly hydrophobic residues such as leucine and valine. In some aspects, a triplet of phenylalanine, tryptophan and valine will be found at each end of a synthetic transmembrane domain. In some embodiments, the linkage is by linkers, spacers, and/or transmembrane domain(s). In some aspects, the transmembrane domain contains a transmembrane portion of CD28 or a variant thereof. The extracellular region and transmembrane can be linked directly or indirectly. In some embodiments, the extracellular region and transmembrane are linked by a spacer, such as any described herein.

In some embodiments, the transmembrane domain of the receptor, e.g., the CAR is a transmembrane domain of human CD28 or variant thereof, e.g., a 27-amino acid transmembrane domain of a human CD28 (Accession No.: P10747.1), or is a transmembrane domain that comprises the sequence of amino acids set forth in SEQ ID NO: 8 or a sequence of amino acids that exhibits at least or at least about 85%, 86%, 87%, 88%, 89%, 90%, 91%, 92%, 93%, 94%, 95%, 96%, 97%, 98%, 99% or more sequence identity to SEQ ID NO:8; in some embodiments, the transmembrane-domain containing portion of the recombinant receptor comprises the sequence of amino acids set forth in SEQ ID NO: 9 or a sequence of amino acids having at least or at least about 85%, 86%, 87%, 88%, 89%, 90%, 91%, 92%, 93%, 94%, 95%, 96%, 97%, 98%, 99% or more sequence identity thereto.

c. Intracellular Region

In some aspects, the recombinant receptor, e.g., CAR, encoded in the modified TGFBR2 locus, includes an intracellular region (also called cytoplasmic region) that comprises a signaling region or domain. In some embodiments, the intracellular region comprises an intracellular signaling region or domain. In some embodiments, the intracellular signaling region or domain is or comprises a primary signaling region, a signaling domain that is capable of stimulating and/or inducing a primary activation signal in a T cell, a signaling domain of a T cell receptor (TCR) component (e.g. an intracellular signaling domain or region of a CD3-zeta (CD3ζ) chain or a functional variant or signaling portion thereof), and/or a signaling domain comprising an immunoreceptor tyrosine-based activation motif (ITAM).

In some embodiments, the recombinant receptor, e.g., CAR, includes at least one intracellular signaling component or components, such as an intracellular signaling region or domain. Among the intracellular signaling region are those that mimic or approximate a signal through a natural antigen receptor, a signal through such a receptor in combination with a costimulatory receptor, and/or a signal through a costimulatory receptor alone. In some embodiments, a short oligo- or polypeptide linker, for example, a linker of between 2 and 10 amino acids in length, such as one containing glycines and serines, e.g., glycine-serine doublet, is present and forms a linkage between the transmembrane domain and the cytoplasmic signaling domain of the CAR.

In some embodiments, upon ligation of the CAR, the cytoplasmic (or intracellular) domain or regions, e.g., intracellular signaling region, of the CAR stimulates and/or activates at least one of the normal effector functions or responses of the immune cell, e.g., T cell engineered to express the CAR. For example, in some contexts, the CAR induces a function of a T cell such as cytolytic activity or T-helper activity, such as secretion of cytokines or other factors. In some embodiments, a truncated portion of an intracellular signaling region or domain of an antigen receptor component or costimulatory molecule is used in place of an intact immunostimulatory chain, for example, if it transduces the effector function signal. In some embodiments, the intracellular signaling regions, e.g., comprising intracellular domain or domains, include the cytoplasmic sequences of a T cell receptor (TCR), and in some aspects also those of co-receptors that in the natural context act in concert with such receptor to initiate signal transduction following antigen receptor engagement, and/or any derivative or variant of such molecules, and/or any synthetic sequence that has the same functional capability. In some embodiments, the intracellular signaling regions, e.g., comprising intracellular domain or domains, include the cytoplasmic sequences of a region or domain that is involved in providing costimulatory signal.

(i) Costimulatory Signaling Domain

In some embodiments, to promote full stimulation and/or activation, one or more components for generating secondary or costimulatory signal is included in the encoded CAR. In other embodiments, the encoded CAR does not include a component for generating a costimulatory signal. In some aspects, an additional receptor polypeptide or portion thereof is expressed in the same cell and provides the component for generating the secondary or costimulatory signal.

In some embodiments, the encoded CAR includes a signaling region and/or transmembrane portion of a costimulatory receptor, such as CD28, 4-1BB, OX40 (CD134), CD27, DAP10, DAP12, ICOS and/or other costimulatory receptors. In some aspects, the same CAR includes both the primary cytoplasmic signaling region and costimulatory signaling components.

In some embodiments, one or more different recombinant receptors can contain one or more different intracellular signaling region(s) or domain(s). In some embodiments, the primary cytoplasmic signaling region is included within one encoded CAR, whereas the costimulatory component is provided by another receptor, e.g., another CAR recognizing another antigen. In some embodiments, the encoded CARs include activating or stimulatory CARs, and costimulatory CARs, both expressed on the same cell (see WO2014/055668).

In certain embodiments, the intracellular signaling region comprises a CD28 transmembrane and signaling domain linked to a CD3 (e.g., CD3ζ) intracellular region or domain. In some embodiments, the intracellular region comprises a chimeric CD28 and CD137 (4-1BB, TNFRSF9) co-stimulatory domains, linked to a CD3ζ intracellular region or domain.

In some embodiments, the encoded CAR encompasses one or more, e.g., two or more, costimulatory domains and primary cytoplasmic signaling region, in the cytoplasmic portion. Exemplary CARs include intracellular components, such as intracellular signaling region(s) or domain(s), of CD3-zeta, CD28, CD137 (4-1BB), OX40 (CD134), CD27, DAP10, DAP12, NKG2D and/or ICOS. In some embodiments, the chimeric antigen receptor contains an intracellular signaling region or domain of a T cell costimulatory molecule, e.g., from CD28, CD137 (4-1BB), OX40 (CD134), CD27, DAP10, DAP12, NKG2D and/or ICOS, in some cases, between the transmembrane domain and intracellular signaling region or domain. In some aspects, the T cell costimulatory molecule is one or more of CD28, CD137 (4-1BB), OX40 (CD134), CD27, DAP10, DAP12, NKG2D and/or ICOS. In some embodiments, the costimulatory molecule is a human costimulatory molecule.

In some embodiments, the intracellular signaling region or domain comprises an intracellular costimulatory signaling domain of human CD28 or functional variant or portion thereof, such as a 41 amino acid domain thereof and/or such a domain with an LL to GG substitution at positions 186-187 of a native CD28 protein. In some embodiments, the intracellular signaling domain can comprise the sequence of amino acids set forth in SEQ ID NO: 10 or 11 or a sequence of amino acids that exhibits at least or at least about 85%, 86%, 87%, 88%, 89%, 90%, 91%, 92%, 93%, 94%, 95%, 96%, 97%, 98%, 99% or more sequence identity to SEQ ID NO: 10 or 11. In some embodiments, the intracellular region comprises an intracellular costimulatory signaling domain or region of CD137(4-1BB) or functional variant or portion thereof, such as a 42-amino acid cytoplasmic domain of a human 4-1BB (Accession No. Q07011.1) or functional variant or portion thereof, such as the sequence of amino acids set forth in SEQ ID NO: 12 or a sequence of amino acids that exhibits at least or at least about 85%, 86%, 87%, 88%, 89%, 90%, 91%, 92%, 93%, 94%, 95%, 96%, 97%, 98%, 99% or more sequence identity to SEQ ID NO: 12.

In some cases, the encoded CARs are referred to as first, second, third or fourth generation CARs. In some aspects, a first generation CAR is one that solely provides a primary stimulation or activation signal, e.g., via CD3-chain induced signal upon antigen binding; in some aspects, a second-generation CAR is one that provides such a signal and costimulatory signal, such as one including an intracellular signaling region(s) or domain(s) from one or more costimulatory receptor such as CD28, CD137 (4-1BB), OX40 (CD134), CD27, DAP10, DAP12, NKG2D, ICOS and/or other costimulatory receptors; in some aspects, a third generation CAR is one that includes multiple costimulatory domains of different costimulatory receptors, e.g., selected from CD28, CD137 (4-1BB), OX40 (CD134), CD27, DAP10, DAP12, NKG2D, ICOS and/or other costimulatory receptors; in some aspects, a fourth generation CAR is one that includes three or more costimulatory domains of different costimulatory receptors, e.g., selected from CD28, CD137 (4-1BB), OX40 (CD134), CD27, DAP10, DAP12, NKG2D, ICOS and/or other costimulatory receptors.

(ii) Primary Signaling Region, e.g., CD3ζ Chain

In some embodiments, the encoded recombinant receptor, e.g., CAR, includes an intracellular component of a TCR complex, such as a TCR CD3 chain that mediates T-cell activation and cytotoxicity, e.g., CD3 zeta chain. Thus, in some aspects, the antigen-binding or antigen-recognition domain is linked to one or more cell signaling modules. In some embodiments, cell signaling modules include CD3 transmembrane domain, CD3 intracellular signaling domains, and/or other CD transmembrane domains. In some embodiments, the encoded recombinant receptor, e.g., CAR, further includes one or more additional molecules such as Fc receptor gamma (FcRγ), CD8 alpha, CD8 beta, CD4, CD25 or CD16. For example, in some aspects, the CAR includes a chimeric molecule between CD3 zeta (CD3ζ) and one or more of CD8 alpha, CD8 beta, CD4, CD25 or CD16.

In the context of a natural TCR, full stimulation generally requires not only signaling through the TCR, but also a costimulatory signal. T cell stimulation is in some aspects can be mediated by two classes of cytoplasmic signaling sequences: those that initiate antigen-dependent primary activation through the TCR (primary cytoplasmic signaling region(s) or domain(s)), and those that act in an antigen-independent manner to provide a secondary or co-stimulatory signal (secondary cytoplasmic signaling region(s) or domain(s)). In some aspects, the CAR includes one or both of such signaling components.

In some aspects, the encoded CAR includes an intracellular region comprising a primary cytoplasmic signaling region that regulates primary stimulation and/or activation of the TCR complex. Primary cytoplasmic signaling region(s) that act in a stimulatory manner may contain signaling motifs which are known as immunoreceptor tyrosine-based activation motifs or ITAMs, e.g., derived from CD3 zeta (CD3ζ). In some embodiments, the CAR contain(s) a cytoplasmic signaling domain, fragment or portion thereof, or sequence derived from CD3ζ. In some embodiments, the intracellular (or cytoplasmic) signaling region comprises a human CD3 zeta chain or a fragment or portion thereof, including the intracellular or cytoplasmic stimulatory signaling domain of CD3ζ or functional variant thereof, such as an 112 AA cytoplasmic domain of isoform 3 of human CD3ζ (Accession No.: P20963.2) or a CD3ζ signaling domain as described in U.S. Pat. Nos. 7,446,190 or 8,911,993. In some embodiments, the intracellular region of the encoded recombinant receptor comprises the sequence of amino acids set forth in SEQ ID NO: 13, 14 or 15 or a sequence of amino acids that exhibits at least or at least about 85%, 86%, 87%, 88%, 89%, 90%, 91%, 92%, 93%, 94%, 95%, 96%, 97%, 98%, 99% or more sequence identity to SEQ ID NO: 13, 14 or 15 or a partial sequence thereof. In some embodiments, exemplary CD3ζ chain or a fragment thereof encoded by the modified TGFBR2 locus include the ITAM domains of the CD3ζ chain, e.g., amino acid residues 61-89, 100-128 or 131-159 of the human CD3ζ chain precursor sequence set forth in SEQ ID NO:188 or a sequence of amino acids that containing one or more ITAM domains from the CD3ζ chain and exhibits at least or at least about 85%, 86%, 87%, 88%, 89%, 90%, 91%, 92%, 93%, 94%, 95%, 96%, 97%, 98%, 99% or more sequence identity to SEQ ID NO: 188.

In some embodiments, the cell is engineered to express one or more additional molecules (e.g., polypeptides, such as an additional recombinant receptor polypeptides or portion thereof) are used to regulate, control, or modulate function and/or activity of the encoded CAR. Exemplary multi-chain recombinant receptors, such as multi-chain CARs, and are described herein, for example, in Section III.B.2.

In some embodiments, the encoded CAR contains an antibody, e.g., an antibody fragment, a transmembrane domain that is or contains a transmembrane portion of CD28 or a functional variant thereof, and an intracellular signaling region containing a signaling portion of CD28 or functional variant thereof and a signaling portion of CD3 zeta or functional variant thereof. In some embodiments, the CAR contains an antibody, e.g., antibody fragment, a transmembrane domain that is or contains a transmembrane portion of CD28 or a functional variant thereof, and an intracellular signaling domain containing a signaling portion of a 4-1BB or functional variant thereof and a signaling portion of CD3 zeta or functional variant thereof. In some such embodiments, the receptor further includes a spacer containing a portion of an Ig molecule, such as a human Ig molecule, such as an Ig hinge, e.g. an IgG4 hinge, such as a hinge-only spacer. In some embodiments, the recombinant receptor comprises a CD3 zeta (CD3ζ) at the C-terminus of the receptor.

2. Multi-Chain CARs

In some embodiments, the recombinant receptor encoded by the nucleic acid sequences of the modified TGFBR2 locus can be a multi-chain CAR. In some embodiments, if the multi-chain CAR comprising two or more polypeptide chains is expressed in the cell, at least one of the polypeptide chains encoded by the modified TGFBR2 locus. In some aspects, the polynucleotide used to introduce nucleic acid sequences encoding one or more chains of the multi-chain CAR can include any described in Section I.B herein. In some aspects, a polynucleotide, e.g., template polynucleotide, contains transgene sequences encoding at least one chain of the multi-chain CAR or a portion thereof, such as at least a portion of at least one polypeptide of a multi-chain CAR. In some aspects, the transgene sequence also includes sequences encoding a different or additional polypeptide, e.g., the other or additional chain of the multi-chain CAR, or additional molecules, such as those described in Section I.B.2.(iv) herein. In some aspects, an additional polynucleotide, e.g., an additional template polynucleotide, can be introduced, that encodes additional components of the multi-chain CAR. In some aspects, the additional polynucleotide can be any polynucleotide described herein, e.g., in Section I.B.2, or a modified form thereof, such as one comprising different homology arms for targeting the nucleic acid for integration at a distinct genomic locus.

In some embodiments, the provided engineered cells include cells that express multi-chain receptors, such as multi-chain CARs In some embodiments, exemplary multi-chain CARs can contain two or more genetically engineered receptors on the cell, which together can comprise a functional recombinant receptor. In some aspects, the various polypeptide chains in combination can perform functions or activities of a CAR, and/or regulate, control, or modulate function and/or activity of the CAR. In some aspects, a multi-chain CAR can contain two or more polypeptide chains, each recognizing the same of a different antigen and typically each including different regions or domains, such as a different intracellular signaling component. In some aspects, the modified TGFBR2 locus can include nucleic acid sequences encoding at least one chain of a multi-chain receptor, such as a multi-chain CAR.

In some embodiments, the recombinant receptor is multi-chain CAR or a dual-chain CAR, that comprises two or more polypeptide chains. In some embodiments, the multi-chain receptor is a regulatable CAR, a conditionally active CAR or an inducible CAR. In some aspects, two or more polypeptides of the recombinant receptor, such as a dual-chain CAR, allows spatial or temporal regulation or control of specificity, activity, antigen (or ligand) binding, function and/or expression of the recombinant receptors. In some of such embodiments, the recombinant receptor encoded by the nucleic acid sequences at the modified TGFBR2 locus can include one or more chains of the dual-chain or multi-chain receptors. In some aspects, in cases where only one of the dual-chain CAR is encoded by the modified TGFBR2 locus, the other chain can be encoded by a separate nucleic acid molecule that is integrated at a different genomic location or is episomal.

In some embodiments, the multi-chain CARs can include combinations of activating and costimulatory CARs. For example, in some embodiments, the multi-chain CAR can include two polypeptides encoding CARs targeting two different antigens present individually on non-target cells, e.g., normal cells, but present together only on cells of the disease or condition to be treated. In some embodiments, the multi-chain CARs can include an activating and an inhibitory CAR, such as those in which the activating CAR binds to one antigen expressed on both normal or non-diseased cells and cells of the disease or condition to be treated, and the inhibitory CAR binds to another antigen expressed only on the normal cells or cells which it is not desired to treat. In some aspects, multi-chain CARs can include one or more polypeptides encoding CARs that are capable of being regulated, modulated or controlled.

In some embodiments, the multi-chain CAR includes one or more polypeptide chains encode one or more domains or regions of a CAR. In some aspects, various polypeptide chains in combination can comprise a CAR. In some embodiments, one or more additional domains or regions are present in the CAR. In some embodiments, various domains or regions present in one or more polypeptide chains of the multi-chain CAR are used to regulate, control, or modulate function and/or activity of the CAR. In some embodiments, the engineered cells express two or more polypeptide chains that contain different components, domains or regions. In some aspects, two or more polypeptide chains allows spatial or temporal regulation or control of specificity, activity, antigen (or ligand) binding, function and/or expression of the recombinant receptors. In some embodiments of the multi-chain CAR including more than one polypeptides, e.g., 2 or more polypeptides, the nucleic acid sequence encoding at least one polypeptide, is targeted for integration at the endogenous TGFBR2 locus. In some embodiments, the nucleic acid sequence encoding an additional molecule or polypeptide, e.g., additional polypeptide chain of the multi-chain CAR or an additional molecule, can be targeted at the same locus, e.g. by virtue of placement on the same polynucleotide used for targeting. In some nucleic acid sequence encoding an additional molecule or polypeptide is targeted at a different locus or is delivered by different methods.

In some aspects, one or more polypeptide chain encoding domains or regions of a CAR can target one or more antigens or molecules. Exemplary multi-chain CARs or other multi-targeting strategies include those described in, for example, in International Pat. App. Pub. No. WO 2014055668 or Fedorov et al., Sci. Transl. Medicine, Sci Transl Med. (2013) 5(215):215ra172; Sadelain, Curr Opin Immunol. (2016) 41: 68-76; Wang et al. (2017) Front. Immunol. 8:1934; Mirzaei et al. (2017) Front. Immunol. 8:1850; Marin-Acevedo et al. (2018) Journal of Hematology & Oncology 11:8; Fesnak et al. (2016) Nat Rev Cancer. 16(9): 566-581; and Abate-Daga and Davila, (2016) Molecular Therapy—Oncolytics 3, 16014.

In some embodiments, the engineered cells can express a first polypeptide chain of the recombinant receptor, e.g., CAR, which is capable of inducing an activating or stimulating signal to the cell, generally upon specific binding to the antigen recognized by the first polypeptide chain, e.g., the first antigen. In some embodiments, the cell can further express a second polypeptide chain of the recombinant receptor, e.g., CAR, in some cases called a chimeric costimulatory receptor, which is capable of inducing a costimulatory signal to the immune cell, generally upon specific binding to a second antigen recognized by the second polypeptide chain. In some embodiments, the first antigen and second antigen are the same. In some embodiments, the first antigen and second antigen are different.

In some embodiments, the first and/or second polypeptide chain is capable of inducing an activating or stimulating signal to the cell. In some embodiments, the receptor includes an intracellular signaling component containing ITAM or ITAM-like motifs. In some embodiments, the activation induced by the first polypeptide chain involves a signal transduction or change in protein expression in the cell resulting in initiation of an immune response, such as ITAM phosphorylation and/or initiation of ITAM-mediated signal transduction cascade, formation of an immunological synapse and/or clustering of molecules near the bound receptor (e.g., CD4 or CD8, etc.), activation of one or more transcription factors, such as NF-κB and/or AP-1, and/or induction of gene expression of factors such as cytokines, proliferation, and/or survival. In some embodiments, the activating domain is included within at least one of the multi-chain CAR, such as the polypeptide chain that is encoded by the modified TGFBR2 locus, whereas the costimulatory component is provided by another polypeptide recognizing another antigen. In some embodiments, the engineered cells can include multi-chain CARs, including activating or stimulatory CARs, costimulatory CARs, both expressed on the same cell (see WO2014/055668). In some aspects, the cells express one or more stimulatory or activating CAR (such as those encoded by the modified TGFBR2 locus as described herein, e.g., in Section III.A) and/or a costimulatory CAR.

In some embodiments, the first and/or second polypeptide chain, includes intracellular signaling regions or domains of costimulatory receptors such as CD28, CD137 (4-1BB), OX40 (CD134), CD27, DAP10, DAP12, NKG2D, ICOS and/or other costimulatory receptors. In some embodiments, the first and second polypeptide chains can contain intracellular signaling domain(s) of a costimulatory receptor that are different. In one embodiment, the first polypeptide chain contains a CD28 costimulatory signaling domain and the second polypeptide chain contain a 4-1BB co-stimulatory signaling region or vice versa.

In some embodiments, the first and/or second polypeptide chain includes both an intracellular signaling domain containing ITAM or ITAM-like motifs, such as those from a CD3zeta (CD3ζ) chain or a fragment or portion thereof, such as the CD3ζ intracellular signaling domain and an intracellular signaling domain of a costimulatory receptor. In some embodiments, the first polypeptide chain contains an intracellular signaling domain containing ITAM or ITAM-like motifs and the second polypeptide chain contains an intracellular signaling domain of a costimulatory receptor. The costimulatory signal in combination with the activating or stimulating signal induced in the same cell is one that results in an immune response, such as a robust and sustained immune response, such as increased gene expression, secretion of cytokines and other factors, and T cell mediated effector functions such as cell killing.

In some embodiments, neither ligation of the first polypeptide chain alone nor ligation of the second polypeptide chain alone induces a robust immune response. In some aspects, if only one receptor is ligated, the cell becomes tolerized or unresponsive to antigen, or inhibited, and/or is not induced to proliferate or secrete factors or carry out effector functions. In some such embodiments, however, when the multiple polypeptide chains are ligated, such as upon encounter of a cell expressing the first and second antigens, a desired response is achieved, such as full immune activation or stimulation, e.g., as indicated by secretion of one or more cytokine, proliferation, persistence, and/or carrying out an immune effector function such as cytotoxic killing of a target cell.

In some embodiments, one or more chain of the multi-chain CAR can include inhibitory CARs (iCARs, see Fedorov et al., Sci. Transl. Medicine, 5(215) (2013), such as a CAR recognizing an antigen other than the one associated with and/or specific for the disease or condition whereby an activating signal delivered through the disease-targeting CAR is diminished or inhibited by binding of the inhibitory CAR to its ligand, e.g., to reduce off-target effects. In some embodiments, the inhibitory CAR can be encoded by the same polynucleotide as the stimulating or activating CAR (e.g., containing a CD3zeta (CD3ζ) chain or a fragment or portion thereof), or by a different polynucleotide.

In some embodiments, the two polypeptide chains of the multi-chain CAR induce, respectively, an activating and an inhibitory signal to the cell, such that ligation of one polypeptide chain to its antigen activates the cell or induces a response, but ligation of the second polypeptide chain, e.g., an inhibitory receptor, to its antigen induces a signal that suppresses or dampens that response. Examples are combinations of activating CARs and inhibitory CARs (iCARs). Such a strategy may be used, for example, to reduce the likelihood of off-target effects in the context in which the activating CAR binds an antigen expressed in a disease or condition but which is also expressed on normal cells, and the inhibitory receptor binds to a separate antigen which is expressed on the normal cells but not cells of the disease or condition.

In some aspects, an additional receptor polypeptide expressed in the cell further includes an inhibitory CAR (e.g. iCAR) and includes intracellular components that dampen or suppress an immune response, such as an ITAM- and/or co stimulatory-promoted response in the cell. Exemplary of such intracellular signaling components are those found on immune checkpoint molecules, including PD-1, CTLA4, LAG3, BTLA, OX2R, TIM-3, TIGIT, LAIR-1, PGE2 receptors, EP2/4 Adenosine receptors including A2AR. In some aspects, the engineered cell includes an inhibitory CAR including a signaling domain of or derived from such an inhibitory molecule, such that it serves to dampen the response of the cell, for example, that induced by an activating and/or costimulatory CAR.

In some embodiments, a multi-chain CAR can be employed where an antigen associated with a particular disease or condition is expressed on a non-diseased cell and/or is expressed on the engineered cell itself, either transiently (e.g., upon stimulation in association with genetic engineering) or permanently. In such cases, by requiring ligation of two separate and individually specific polypeptides, specificity, selectivity, and/or efficacy may be improved.

In some embodiments, the plurality of antigens, e.g., the first and second antigens, are expressed on the cell, tissue, or disease or condition being targeted, such as on the cancer cell. In some aspects, the cell, tissue, disease or condition is multiple myeloma or a multiple myeloma cell. In some embodiments, one or more of the plurality of antigens generally also is expressed on a cell which it is not desired to target with the cell therapy, such as a normal or non-diseased cell or tissue, and/or the engineered cells themselves. In such embodiments, by requiring ligation of multiple receptors to achieve a response of the cell, specificity and/or efficacy is achieved.

In some embodiments, one of the first and/or second polypeptide chains can regulate the expression, antigen binding and/or activity of the other polypeptide chain.

In some aspects, a two polypeptide chain system can be used to regulate the expression of at least one of the polypeptide chains. In some embodiments, the first polypeptide chain contains a first ligand- (e.g., antigen-) binding domain linked to a regulatory molecule, such as a transcription factor, linked via a regulatable cleavage element. In some aspects, the regulatable cleavage element is derived from a modified Notch receptor (e.g., synNotch), which is capable of cleaving and releasing an intracellular domain upon engagement of the first ligand- (e.g., antigen-) biding domain. In some aspects, the second polypeptide chain contains a second ligand- (e.g., antigen-) binding domain linked to an intracellular signaling component capable of inducing an activating or stimulating signal to the cell, such as an ITAM-containing intracellular signaling domain. In some aspects, the nucleic acid sequence encoding the second polypeptide chain is operably linked to transcriptional regulatory elements, e.g., promoter, that is capable of being regulated by a particular transcription factor, e.g., transcription factor encoded by the first polypeptide chain. In some aspects, engagement of a ligand or an antigen to the first ligand- (e.g., antigen-) binding domain leads to proteolytic release of the transcription factor, which in turn can induce the expression of the second polypeptide chain (see Roybal et al. (2016) Cell 164:770-779; Morsut et al. (2016) Cell 164:780-791). In some embodiments, the first antigen and second antigen are different.

In some instances, the recombinant receptor, e.g., CAR, is capable of being regulated, controlled, induced or inhibited, can be desirable to optimize the safety and efficacy of a therapy with the recombinant receptor. In some embodiments, the multi-chain CAR is a regulatable CAR. In some aspects, provided herein is an engineered cell comprising a CAR that is capable of being regulated. A recombinant receptor that is capable of being regulated, also referred to herein as a “regulatable recombinant receptor,” or a “regulatable CAR” refers to multiple polypeptides, such as a set of at least two polypeptide chains, which when expressed in an engineered cell (e.g., engineered T cell), provides the engineered cell with the ability to generate an intracellular signal under the control of an inducer.

In some embodiments, the polypeptides of the regulatable CAR contain multimerization domains that are capable of multimerization with another multimerization domain. In some embodiments, the multimerization domain is capable of multimerization upon binding to an inducer. For example, the multimerization domain can bind an inducer, such as a chemical inducer, which results in multimerization of the polypeptides of the regulatable CAR by virtue of multimerization of the multimerization domain, thereby producing the regulatable CAR.

In some embodiments, one polypeptide of the regulatable CAR comprises a ligand- (e.g., antigen-) binding domain and a different polypeptide of the regulatable CAR comprises an intracellular signaling region, wherein multimerization of the two polypeptides by virtue of multimerization of the multimerization domain produces a regulatable CAR comprising a ligand-binding domain and an intracellular signaling region. In some embodiments, multimerization can induce, modulate, activate, mediate and/or promote signals in the engineered cell containing the regulatable CAR. In some embodiments, an inducer binds to a multimerization domain at least one polypeptide of a regulatable CAR and induces a conformational change of the regulatable CAR, wherein the conformational change activates signaling. In some embodiments, binding of a ligand to such chimeric receptors induces conformational changes in the polypeptide chain, including, in some cases, polypeptide chain oligomerization, which can render the receptors competent for intracellular signaling.

In some embodiments, an inducer functions to couple or multimerize (e.g., dimerize) a set of at least two polypeptide chains of a regulatable CAR expressed in an engineered cell in order for the regulatable CAR to produce a desired intracellular signal such as during interaction of the regulatable CAR with a target antigen. Coupling or multimerization of at least two polypeptides of a regulatable CAR by an inducer is achieved upon binding of an inducer to a multimerization domain. For example, in some embodiments, a first polypeptide and a second polypeptide in an engineered cell may each comprise a multimerization domain capable of binding an inducer. Upon binding of the multimerization domain by the inducer, the first polypeptide and the second polypeptide are coupled together to produce the desired intracellular signal. In some embodiments, a multimerization domain is located on an intracellular portion of a polypeptide. In some embodiments, a multimerization domain is located on an extracellular portion of a polypeptide.

In some embodiments, a set of at least two polypeptides of a regulatable CAR comprises two, three, four, or five or more polypeptides. In some embodiments, the set of at least two polypeptides are the same polypeptides, for example, two, three, or more of the same polypeptides comprising an intracellular signaling region, and a multimerization domain. In some embodiments, the set of at least two polypeptides are different polypeptides, for example, a first polypeptide comprising an ligand- (e.g., antigen-) binding domain and a multimerization domain and a second polypeptide comprising an intracellular signaling region and a multimerization domain. In some embodiments, the intercellular signal is generated in the presence of an inducer. In some embodiments, the intracellular signal is generated in the absence of an inducer, e.g., an inducer interferes with multimerization of at least two polypeptides of a regulatable CAR thereby preventing intracellular signaling by the regulatable CAR.

In some embodiments, the multi-chain CAR, the nucleic acid sequence encoding at least one of the polypeptide chains, is integrated into the endogenous TGFBR2 locus, e.g., by HDR. In some embodiments, the nucleic acid sequences encoding the other of the two or more separate polypeptide chains, can be targeted within the same locus (e.g., within the same transgene sequence, and can be placed 5′ or 3′ of the nucleic acid sequence encoding the other polypeptide chain), or at a different locus. In some aspects, the introduction of the nucleic acid sequences encoding the other of the two or more separate polypeptide chains may be via different delivery methods, e.g., by transient delivery methods or as an episomal nucleic acid molecule.

In some embodiments, one or more of the polypeptide chains of a multi-chain CAR, can include a multimerization domain. In some embodiments, the multimerization domain can multimerize (e.g., dimerize), upon binding of an inducer. An inducer contemplated herein includes, but is not limited to, a chemical inducer or a protein (e.g., a caspase). In some embodiments, the inducer is selected from an estrogen, a glucocorticoid, a vitamin D, a steroid, a tetracycline, a cyclosporine, Rapamycin, Coumermycin, Gibberellin, FK1012, FK506, FKCsA, rimiducid or HaXS, or analogs or derivatives thereof. In some embodiments, the inducer is AP20187 or an AP20187 analog, such as, AP1510.

In some embodiments, the multimerization domain can multimerize (e.g., dimerize), upon binding of an inducer such as an inducer provided herein. In some embodiments, the multimerization domain can be from an FKBP, a cyclophilin receptor, a steroid receptor, a tetracycline receptor, an estrogen receptor, a glucocorticoid receptor, a vitamin D receptor, Calcineurin A, CyP-Fas, FRB domain of mTOR, GyrB, GAI, GID1, Snap-tag and/or HaloTag, or portions or derivatives thereof. In some embodiments, the multimerization domain is an FK506 binding protein (FKBP) or derivative thereof, or fragment and/or multimer thereof, such as FKBP12v36. In some embodiments, FKBP comprises the amino acid sequence

(SEQ ID NO: 82)
GVQVETISPGDGRTFPKRGQTCVVHYTGMLEDGKKMDSSRDRNKPFKFML
GKQEVIRGWEEGVAQMSVGQRAKLTISPDYAYGATGHPGIIPPHATLVFD
VELLKLE.

In some embodiments, FKBP12v36 comprises the amino acid sequence

(SEQ ID NO: 83)
GVQVETISPGDGRTFPKRGQTCVVHYTGMLEDGKKVDSSRDRNKPFKFM
LGKQEVIRGWEEGVAQMSVGQRAKLTISPDYAYGATGHPGIIPPHATLV
FDVELLKLE.

Exemplary inducers and corresponding multimerization domains are known, e.g., as described in U.S. Pat. App. Pub. No. 2016/0046700, Clackson et al. (1998) Proc Natl Acad Sci USA. 95(18):10437-42; Spencer et al. (1993) Science 262(5136):1019-24; Farrar et al. (1996) Nature 383 (6596):178-81; Miyamoto et al. (2012) Nature Chemical Biology 8(5): 465-70; Erhart et al. (2013) Chemistry and Biology 20(4): 549-57). In some embodiments, the inducer is rimiducid (also known as AP1903; CAS Index Name: 2-Piperidinecarboxylic acid, 1-[(2S)-1-oxo-2-(3,4,5-trimethoxyphenyl)butyl]-, 1,2-ethanediylbis [imino (2-oxo-2, 1-ethanediyl)oxy-3,1-phenylene[(1R)-3-(3,4-Dimethoxyphenyl)propylidene]]ester, [2S-[1(R*),2R*[S*[S*[1(R*),2R]]]]]-(9Cl); CAS Registry Number: 195514-63-7; Molecular Formula: C₇₈H₉₈N₄O₂₀; Molecular Weight: 1411.65), and the multimerization domain is an FK506 binding protein (FKBP).

In some embodiments, the cell membrane of the engineered cell is impermeable to the inducer. In some embodiments, the cell membrane of the engineered cell is permeable to the inducer.

In some embodiments, the regulatable CAR are not part of a multimer or a dimer in the absence of the inducer. Upon the binding of the inducer, the multimerization domains can multimerize, e.g., dimerize. In some aspects, multimerization of the multimerization domain results in multimerization of a polypeptide of the regulatable CAR with another polypeptide of the regulatable CAR, e.g. multimeric complex of at least two polypeptides of the regulatable CARs. In some embodiments, multimerization of the multimerization domain can induce, modulate, activate, mediate and/or promote signal transduction by virtue of inducing physical proximity of signaling components or formation of the multimer or dimer. In some embodiments, upon the binding of an inducer, multimerization of the multimerization domain also induces multimerization of signaling domains linked, directly or indirectly, to the multimerization domain. In some embodiments, the multimerization induces, modulates, activates, mediates and/or promotes signaling through the signaling domain or region. In some embodiments, the signaling domain or region linked to the multimerization domain is an intracellular signaling region.

In some embodiments, the multimerization domain is intracellular or is associated with the cell membrane on the intracellular or cytoplasmic side of the engineered cell (e.g., engineered T cell). In some aspects, the intracellular multimerization domain is linked, directly or indirectly, to a membrane association domain (e.g., a lipid linking domain), such as a myristoylation domain, palmitoylation domain, prenylation domain, or a transmembrane domain. In some embodiments, the multimerization domain is intracellular, and is linked to the extracellular ligand- (e.g., antigen-) binding domain via a transmembrane domain. In some embodiments, the intracellular multimerization domain is linked, directly or indirectly, to the intracellular signaling region. In some aspects, induced multimerization of the multimerization domain also brings the intracellular signaling regions in proximity with one another, to allow multimerization, e.g., dimerization, and stimulate intracellular signaling. In some embodiments, a polypeptide of the regulatable CAR comprises a transmembrane domain, one or more intracellular signaling region(s), and one or more multimerization domain(s), each of which are linked directly or indirectly.

In some embodiments, the multimerization domain is extracellular or is associated with the cell membrane on the extracellular side of the engineered cell (e.g., engineered T cell). In some aspects, the extracellular multimerization domain is linked, directly or indirectly, to a membrane association domain (e.g., a lipid linking domain), such as a myristoylation domain, palmitoylation domain, prenylation domain, or a transmembrane domain. In some embodiments, the extracellular multimerization domain is linked, directly or indirectly, to a ligand-binding domain, e.g., an antigen-binding domain such as for binding to an antigen associated with a disease. In some embodiments, the multimerization domain is extracellular, and is linked to an intracellular signaling region via a transmembrane domain.

In some aspects, the membrane association domain is a transmembrane domain of an existing transmembrane protein. In some examples, the membrane association domain is any of the transmembrane domains described herein. In some aspects, the membrane association domain contains protein-protein interaction motifs or transmembrane sequences.

In some aspects, the membrane association domain is an acylation domain, such as a myristoylation domain, palmitoylation domain, prenylation domain (i.e., farnesylation, geranyl-geranylation, CAAX Box). For example, the membrane association domain can be an acylation sequence motif present in N-terminus or C-terminus of a protein. Such domains contain particular sequence motifs that can be recognized by acyltransferases that transfer acyl moieties to the polypeptide that contains the domain. For example, the acylation motifs can be modified with a single acyl moiety (in some cases, followed by several positively charged residues (e.g. human c-Src: MGSNKSKPKDASQRRR (SEQ ID NO:84) to improve association with anionic lipid head groups). In other aspects, the acetylation motif is capable of being modified with multiple acyl moieties. For example, dual acylation regions are located within the N-terminal regions of certain protein kinases, such as a subset of Src family members (e.g., Yes, Fyn, Lek) and G-protein alpha subunits. Exemplary dual acylation regions contain the sequence motif Met-Gly-Cys-Xaa-Cys, (SEQ ID NO:85) where the Met is cleaved, the Gly is N-acylated and one of the Cys residues is S-acylated. The Gly often is myristoylated and a Cys can be palmitoylated.

Other exemplary acylation regions include sequence motif Cys-Ala-Ala-Xaa (so called “CAAX boxes”; SEQ ID NO:86) that can modified with C15 or 010 isoprenyl moieties, and are known (see, e.g., Gauthier-Campbell et al. (2004) Molecular Biology of the Cell 15:2205-2217; Glabati et al. (1994) Biochem. J. 303: 697-700 and Zlakine et al. (1997) J. Cell Science 110:673-679; ten Klooster et al. (2007) Biology of the Cell 99:1-12; Vincent et al. (2003) Nature Biotechnology 21:936-40). In some embodiments, the acyl moiety is a C1-C20 alkyl, C2-C20 alkenyl, C2-C20 alkynyl, C3-C6 cycloalkyl, C1-C4 haloalkyl, C4-C12 cycloalkylalkyl, aryl, substituted aryl, or aryl (C1-C4) alkyl. In some embodiments, the acyl-containing moiety is a fatty acid, and examples of fatty acid moieties are propyl (C3), butyl (C4), pentyl (C5), hexyl (C6), heptyl (C7), octyl (C8), nonyl (C9), decyl (C10), undecyl (C11), lauryl (C12), myristyl (C14), palmityl (C16), stearyl (C18), arachidyl (C20), behenyl (C22) and lignoceryl moieties (C24), and each moiety can contain 0, 1, 2, 3, 4, 5, 6, 7 or 8 unsaturated bonds (i.e., double bonds). In some examples, the acyl moiety is a lipid molecule, such as a phosphatidyl lipid (e.g., phosphatidyl serine, phosphatidyl inositol, phosphatidyl ethanolamine, phosphatidyl choline), sphingolipid (e.g., shingomyelin, sphingosine, ceramide, ganglioside, cerebroside), or modified versions thereof. In certain embodiments, one, two, three, four or five or more acyl moieties are linked to a membrane association domain.

In some aspects, the membrane association domain is a domain that promotes an addition of a glycolipid (also known as glycosyl phosphatidylinositols or GPIs). In some aspects, a GPI molecule is post-translationally attached to a protein target by a transamidation reaction, which results in the cleavage of a carboxy-terminal GPI signal sequence (see, e.g., White et al. (2000) J. Cell Sci. 113:721) and the simultaneous transfer of the already synthesized GPI anchor molecule to the newly formed carboxy-terminal amino acid (See, e.g., Varki A, et al., editors. Essentials of Glycobiology. Cold Spring Harbor (N.Y.): Cold Spring Harbor Laboratory Press; 1999. Chapter 10, Glycophospholipid Anchors. Available from: https://www.ncbi.nlm.nih.gov/books/NBK20711/). In certain embodiments, the membrane association domain is a GPI signal sequence.

In some embodiments, a multimerization domain as provided herein is linked to an intracellular signaling regions, e.g., a primary signaling region and/or costimulatory signaling domains. In some embodiments, the multimerization domain is extracellular, and is linked to the intracellular signaling region via a transmembrane domain. In some embodiments, the multimerization domain is intracellular, and is linked to the ligand- (e.g., antigen-) binding domain via a transmembrane domain. The ligand-binding domain and transmembrane domain can be linked directly or indirectly. In some embodiments, the ligand-binding domain and transmembrane are linked by a spacer, such as any described herein. In some embodiments, the multimerization domain is an FK506 binding protein (FKBP) or derivative or fragment thereof, such as FKBP12v36. In some examples, upon the introduction of an inducer, such as a rimiducid, the polypeptides of the regulatable CAR multimerize, e.g., dimerize, thereby stimulating the signaling domains associated with the multimerization domain and forming a multimeric complex. Formation of the multimeric complex results in inducing, modulating, stimulating, activating, mediating and/or promoting signals through intracellular signaling region.

In some embodiments, signaling through the regulatable CAR can be modulated in a conditional manner through conditional multimerization. For example, the multimerization domain of the polypeptides of the regulatable CAR can bind an inducer to multimerize, and the inducer can be provided exogenously. In some aspects, upon binding of the inducer, the multimerization domain multimerizes and induces, modulates, activates, mediates and/or promotes signaling through the signaling domain. For example, the inducer can be exogenously administered, thereby controlling the location and duration of the signal provided to the engineered cell containing the regulatable CAR. In some embodiments, the multimerization domain of the polypeptides of the regulatable CAR can bind an inducer to multimerize, and the inducer can be provided endogenously. For example, the inducer can be produced endogenously by the engineered cell (e.g., engineered T cell) from a recombinant expression vector or from the genome of the engineered cell under the control of an inducible or conditional promoter, thereby controlling the location and duration of the signal provided to the engineered cell containing the regulatable CAR.

In some embodiments, the regulatable CAR is controlled using a suicide switch. Exemplary chimeric receptors utilize an inducible caspase-9 (iCasp9) system, comprising a fusion of human caspase-9 and a modified FKBP dimerization domain, allowing conditional dimerization upon binding with an inducer, e.g., AP1903. Upon dimerization by binding of the inducer, caspase-9 becomes activated and results in apoptosis and cell death of the cells expressing the chimeric receptor (see, e.g., Di Stasi et al. (2011) N. Engl. J. Med. 365:1673-1683).

In some embodiments, exemplary regulatable CAR includes: (1) a first polypeptide of a regulatable CAR comprising: (i) intracellular signaling region; and (ii) at least one multimerization domain capable of binding an inducer; and (2) a second polypeptide of a regulatable CAR comprising: (i) a ligand- (e.g., antigen-) binding domain; (ii) a transmembrane domain; and (iii) at least one multimerization domain capable of binding an inducer. In some embodiments, exemplary regulatable CAR includes: (1) a first polypeptide of a regulatable CAR comprising: (i) a transmembrane domain or an acylation domain; (ii) intracellular signaling region; and (iii) at least one multimerization domain capable of binding an inducer; and (2) a second polypeptide of a regulatable CAR comprising: (i) a ligand- (e.g., antigen-) binding domain; (ii) a transmembrane domain; and (iii) at least one multimerization domain capable of binding an inducer. In some embodiments, the intracellular signaling region further comprises a costimulatory signaling domain. In some embodiments, the second polypeptide further comprises a costimulatory signaling domain. In some embodiments, the at least one multimerization domain(s) on both polypeptides is intracellular. In some embodiments, the at least one multimerization domain(s) on both polypeptides is extracellular.

In some embodiments, exemplary regulatable CAR includes: (1) a first polypeptide of a regulatable CAR comprising: (i) at least one extracellular multimerization domain capable of binding an inducer; (ii) a transmembrane domain; and (iii) intracellular signaling region; and (2) a second polypeptide of a regulatable CAR comprising: (i) a ligand- (e.g., antigen-) binding domain; (ii) at least one extracellular multimerization domain capable of binding an inducer and (iii) a transmembrane domain, an acylation domain or a GPI signal sequence. In some embodiments, the intracellular signaling region further comprises a costimulatory signaling domain. In some embodiments, the second polypeptide further comprises a costimulatory signaling domain.

In some embodiments, exemplary regulatable CAR includes: (1) a first polypeptide of a regulatable CAR comprising: (i) a transmembrane domain or an acylation domain; (ii) at least one costimulatory domain; (iii) a multimerization domain capable of binding an inducer and (iv) intracellular signaling region; and (iii) at least one costimulatory domain; and (2) a second polypeptide of a regulatable CAR comprising: (i) a ligand- (e.g., antigen-) binding domain; (ii) a transmembrane domain; (iii) at least one costimulatory domain; and (iv) at least one extracellular multimerization domain capable of binding an inducer.

In some aspects, any of the regions and/or domains described in the exemplary regulatable CARs can be ordered in various different orders. In some aspects, the various polypeptides of the regulatable CAR(s) contain the multimerization domain on the same side of the cell membrane, e.g., the multimerization domain in the two or more polypeptides are all intracellular or all extracellular.

Variations of regulatable CARs are known, for example, described in U.S. Pat. App. Pub. No. 2014/0286987, U.S. Pat. App. Pub. No. 2015/0266973, International Pat. App. Pub. No. WO2014/127261, and International Pat. App. Pub. No. WO2015/142675.

3. Chimeric Auto-Antibody Receptor (CAAR)

In some embodiments, the recombinant receptor encoded by the modified TGFBR2 locus is a chimeric autoantibody receptor (CAAR). In some embodiments, the CAAR binds, e.g., specifically binds, or recognizes, an autoantibody. In some embodiments, a cell expressing the CAAR, such as a T cell engineered to express a CAAR, can be used to bind to and kill autoantibody-expressing cells, but not normal antibody expressing cells. In some embodiments, CAAR-expressing cells can be used to treat an autoimmune disease associated with expression of self-antigens, such as autoimmune diseases. In some embodiments, CAAR-expressing cells can target B cells that ultimately produce the autoantibodies and display the autoantibodies on their cell surfaces, mark these B cells as disease-specific targets for therapeutic intervention. In some embodiments, CAAR-expressing cells can be used to efficiently targeting and killing the pathogenic B cells in autoimmune diseases by targeting the disease-causing B cells using an antigen-specific chimeric autoantibody receptor. In some embodiments, the recombinant receptor is a CAAR, such as any described in U.S. Patent Application Pub. No. US 2017/0051035.

In some embodiments, the CAAR comprises an autoantibody binding domain, a transmembrane domain, and one or more intracellular signaling region or domain (also interchangeably called a cytoplasmic signaling domain or region). In some embodiments, the intracellular signaling region comprises an intracellular signaling domain. In some embodiments, the intracellular signaling domain is or comprises a primary signaling region, a signaling domain that is capable of stimulating and/or inducing a primary activation signal in a T cell, a signaling domain of a T cell receptor (TCR) component (e.g. an intracellular signaling domain or region of a CD3-zeta (CD3ζ) chain or a functional variant or signaling portion thereof), and/or a signaling domain comprising an immunoreceptor tyrosine-based activation motif (ITAM).

In some embodiments, the autoantibody binding domain comprises an autoantigen or a fragment thereof. The choice of autoantigen can depend upon the type of autoantibody being targeted. For example, the autoantigen may be chosen because it recognizes an autoantibody on a target cell, such as a B cell, associated with a particular disease state, e.g. an autoimmune disease, such as an autoantibody-mediated autoimmune disease. In some embodiments, the autoimmune disease includes pemphigus vulgaris (PV). Exemplary autoantigens include desmoglein 1 (Dsg1) and Dsg3.

4. T Cell Receptors (TCRs)

In some embodiments, the recombinant receptor encoded by the modified TGFBR2 locus is a T cell receptor (TCR) or portion thereof, such as a recombinant TCR or an antigen-binding portion thereof, that recognizes an intracellular and/or a peptide epitope or T cell epitope of a target polypeptide, such as an antigen of a tumor, viral or autoimmune protein. In some aspects, the encoded receptor is or includes a recombinant TCR. In some aspects, the recombinant TCR is a single-chain TCR or a multi-chain TCR, such as a dual-chain TCR.

In some embodiments, a “T cell receptor” or “TCR” is a molecule that contains a variable a and β chains (also known as TCRα and TCRβ, respectively) or a variable γ and δ chains (also known as TCRγ and TCRδ, respectively), or antigen-binding portions thereof, and which is capable of specifically binding to a peptide bound to an MHC molecule. In some embodiments, the TCR is in the αβ form. In some embodiments, TCRs that exist in αβ and γδ forms are generally structurally similar, but T cells expressing them may have distinct anatomical locations or functions. A TCR can be found on the surface of a cell or in soluble form. In some embodiments, the TCR is a dual-chain TCR, comprising a TCRα and a TCRβ; or a TCRγ and a TCRδ chain. In some aspects, a TCR is found on the surface of T cells (or T lymphocytes) where it is generally responsible for recognizing antigens bound to major histocompatibility complex (MHC) molecules.

In some embodiments, a TCR encompasses a full-length TCRs or antigen-binding portions or antigen-binding fragments thereof. In some embodiments, the TCR is an intact or full-length TCR, including TCRs in the αβ form or γδ form. In some embodiments, the TCR is an antigen-binding portion that is less than a full-length TCR but that binds to a specific peptide bound in an MHC molecule, such as binds to an MHC-peptide complex. In some cases, an antigen-binding portion or fragment of a TCR can contain only a portion of the structural domains of a full-length or intact TCR, but yet is able to bind the peptide epitope, such as MHC-peptide complex, to which the full TCR binds. In some cases, an antigen-binding portion contains the variable domains of a TCR, such as variable α (V_{α}) chain and variable β (V_{β}) chain of a TCR, or antigen-binding fragments thereof sufficient to form a binding site for binding to a specific MHC-peptide complex.

In some embodiments, the variable domains of the encoded TCR contain hypervariable loops, or complementarity determining regions (CDRs), which generally are the primary contributors to antigen recognition and binding capabilities and specificity. In some embodiments, a CDR of a TCR or combination thereof forms all or substantially all of the antigen-binding site of a given TCR molecule. The various CDRs within a variable region of a TCR chain generally are separated by framework regions (FRs), which generally display less variability among TCR molecules as compared to the CDRs (see, e.g., Jores et al., Proc. Nat'l Acad. Sci. U.S.A. 87:9138, 1990; Chothia et al., EMBO J. 7:3745, 1988; see also Lefranc et al., Dev. Comp. Immunol. 27:55, 2003). In some embodiments, CDR3 is the main CDR responsible for antigen binding or specificity, or is the most important among the three CDRs on a given TCR variable region for antigen recognition, and/or for interaction with the processed peptide portion of the peptide-MHC complex. In some contexts, the CDR1 of the alpha chain can interact with the N-terminal part of certain antigenic peptides. In some contexts, CDR1 of the beta chain can interact with the C-terminal part of the peptide. In some contexts, CDR2 contributes most strongly to or is the primary CDR responsible for the interaction with or recognition of the MHC portion of the MHC-peptide complex. In some embodiments, the variable region of the f-chain can contain a further hypervariable region (CDR4 or HVR4), which generally is involved in superantigen binding and not antigen recognition (Kotb (1995) Clinical Microbiology Reviews, 8:411-426).

In some embodiments, the encoded TCR also can contain a constant domain, a transmembrane domain and/or a short cytoplasmic tail (see, e.g., Janeway et al., Immunobiology: The Immune System in Health and Disease, 3rd Ed., Current Biology Publications, p. 4:33, 1997). In some aspects, each chain of the TCR can possess one N-terminal immunoglobulin variable domain, one immunoglobulin constant domain, a transmembrane region, and a short cytoplasmic tail at the C-terminal end. In some embodiments, a TCR is associated with invariant proteins of the CD3 complex involved in mediating signal transduction.

In some embodiments, the encoded TCR chain contains one or more constant domain. For example, the extracellular portion of a given TCR chain (e.g., α-chain or β-chain) can contain two immunoglobulin-like domains, such as a variable domain (e.g., Vα or Vβ; typically amino acids 1 to 116 based on Kabat numbering Kabat et al., “Sequences of Proteins of Immunological Interest, US Dept. Health and Human Services, Public Health Service National Institutes of Health, 1991, 5th ed.) and a constant domain (e.g., α-chain constant domain or Cα, typically positions 117 to 259 of the chain based on Kabat numbering or β chain constant domain or C_{β}, typically positions 117 to 295 of the chain based on Kabat) adjacent to the cell membrane. For example, in some cases, the extracellular portion of the TCR formed by the two chains contains two membrane-proximal constant domains, and two membrane-distal variable domains, which variable domains each contain CDRs. The constant domain of the TCR may contain short connecting sequences in which a cysteine residue forms a disulfide bond, thereby linking the two chains of the TCR. In some embodiments, a TCR may have an additional cysteine residue in each of the α and β chains, such that the TCR contains two disulfide bonds in the constant domains.

In some embodiments, the encoded TCR chains contain a transmembrane domain. In some embodiments, the transmembrane domain is positively charged. In some cases, the TCR chain contains a cytoplasmic tail. In some cases, the structure allows the TCR to associate with other molecules like CD3 and subunits thereof. For example, a TCR containing constant domains with a transmembrane region may anchor the protein in the cell membrane and associate with invariant subunits of the CD3 signaling apparatus or complex. The intracellular tails of CD3 signaling subunits (e.g. CD3γ, CD3δ, CD3ε and CD3ζ chains) contain one or more immunoreceptor tyrosine-based activation motif or ITAM that are involved in the signaling capacity of the TCR complex.

In some embodiments, the encoded TCR contains various domains or regions. In some cases, the exact domain or region can vary depending on the particular structural or homology modeling or other features used to describe a particular domain. It is understood that reference to amino acids, including to a specific sequence set forth as a SEQ ID NO used to describe domain organization of a recombinant receptor, e.g., TCR, are for illustrative purposes and are not meant to limit the scope of the embodiments provided. In some cases, the specific domain (e.g. variable or constant) can be several amino acids (such as one, two, three or four) longer or shorter. In some aspects, residues of a TCR are known or can be identified according to the International Immunogenetics Information System (IMGT) numbering system (see e.g. www.imgt.org; see also, Lefranc et al. (2003) Developmental and Comparative Immunology, 27; 55-77; and The T Cell Factsbook 2nd Edition, Lefranc and LeFranc Academic Press 2001). Using this system, the CDR1 sequences within a TCR Vα chains and/or Vβ chain correspond to the amino acids present between residue numbers 27-38, inclusive, the CDR2 sequences within a TCR Vα chain and/or Vβ chain correspond to the amino acids present between residue numbers 56-65, inclusive, and the CDR3 sequences within a TCR Vα chain and/or Vβ chain correspond to the amino acids present between residue numbers 105-117, inclusive.

In some embodiments, the α chain and β chain of a TCR each further contain a constant domain. In some embodiments, the α chain constant domain (Cα) and β chain constant domain (Cβ) individually are mammalian, such as is a human or murine constant domain. In some embodiments, the constant domain is adjacent to the cell membrane. For example, in some cases, the extracellular portion of the encoded TCR formed by the two chains contains two membrane-proximal constant domains, and two membrane-distal variable domains, which variable domains each contain CDRs.

In some embodiments, each of the Cα and Cβ domains is human. In some embodiments, the Cα is encoded by the TRAC gene (IMGT nomenclature) or is a variant thereof. In some embodiments, the Cβ is encoded by TRBC1 or TRBC2 genes (IMGT nomenclature) or is a variant thereof. In some embodiments, any of the provided TCRs or antigen-binding fragments thereof can be a human/mouse chimeric TCR. In some cases, the encoded TCR or antigen-binding fragment thereof have α chain and/or a β chain comprising a mouse constant region. In some aspects, the Cα and/or Cβ regions are mouse constant regions. In some of any such embodiments, the encoded TCR or antigen-binding fragment thereof is encoded by a nucleotide sequence that has been codon-optimized.

In some of any such embodiments, the binding molecule or TCR or antigen-binding fragment thereof is isolated or purified or is recombinant. In some of any such embodiments, the binding molecule or TCR or antigen-binding fragment thereof is human.

In some embodiments, the encoded TCR may be a heterodimer of two chains α and β that are linked, such as by a disulfide bond or disulfide bonds. In some embodiments, the constant domain of the encoded TCR may contain short connecting sequences in which a cysteine residue forms a disulfide bond, thereby linking the two chains of the encoded TCR. In some embodiments, a TCR may have an additional cysteine residue in each of the α and β chains, such that the encoded TCR contains two disulfide bonds in the constant domains. In some embodiments, each of the constant and variable domains contains disulfide bonds formed by cysteine residues.

In some embodiments, the encoded TCR may be a heterodimer of two chains α and β or γ and δ, such as a dual-chain TCR, or it may be a single chain TCR construct. In some embodiments, the TCR is a heterodimer containing two separate chains (dual-chain TCR, α and β chains or γ and δ chains) that are linked, such as by a disulfide bond or disulfide bonds.

In some embodiments, the encoded TCR can be generated from a known TCR sequence(s), such as sequences of Vα,β chains, for which a substantially full-length coding sequence is readily available. Methods for obtaining full-length TCR sequences, including V chain sequences, from cell sources are well known. In some embodiments, nucleic acids encoding the TCR can be obtained from a variety of sources, such as by polymerase chain reaction (PCR) amplification of TCR-encoding nucleic acids within or isolated from a given cell or cells, or synthesis of publicly available TCR DNA sequences.

In some embodiments, the encoded recombinant receptors include recombinant TCRs and/or TCRs cloned from naturally occurring T cells. In some embodiments, a high-affinity T cell clone for a target antigen (e.g., a cancer antigen) is identified, isolated from a patient, and introduced into the cells. In some embodiments, the TCR clone for a target antigen has been generated in transgenic mice engineered with human immune system genes (e.g., the human leukocyte antigen system, or HLA). See, e.g., tumor antigens (see, e.g., Parkhurst et al. (2009) Clin Cancer Res. 15:169-180 and Cohen et al. (2005) J Immunol. 175:5799-5808. In some embodiments, phage display is used to isolate TCRs against a target antigen (see, e.g., Varela-Rohena et al. (2008) Nat Med. 14:1390-1395 and Li (2005) Nat Biotechnol. 23:349-354.

In some embodiments, the encoded TCR is obtained from a biological source, such as from cells such as from a T cell (e.g. cytotoxic T cell), T-cell hybridomas or other publicly available source. In some embodiments, the T-cells can be obtained from in vivo isolated cells. In some embodiments, the TCR is a thymically selected TCR. In some embodiments, the TCR is a neoepitope-restricted TCR. In some embodiments, the T-cells can be a cultured T-cell hybridoma or clone. In some embodiments, the TCR or antigen-binding portion thereof or antigen-binding fragment thereof can be synthetically generated from knowledge of the sequence of the TCR.

In some embodiments, the encoded TCR is generated from a TCR identified or selected from screening a library of candidate TCRs against a target polypeptide antigen, or target T cell epitope thereof. TCR libraries can be generated by amplification of the repertoire of Vα and Vβ from T cells isolated from a subject, including cells present in PBMCs, spleen or other lymphoid organ. In some cases, T cells can be amplified from tumor-infiltrating lymphocytes (TILs). In some embodiments, TCR libraries can be generated from CD4+ or CD8+ cells. In some embodiments, the TCRs can be amplified from a T cell source of a normal of healthy subject, i.e. normal TCR libraries. In some embodiments, the TCRs can be amplified from a T cell source of a diseased subject, i.e., diseased TCR libraries. In some embodiments, degenerate primers are used to amplify the gene repertoire of Vα and Vβ, such as by RT-PCR in samples, such as T cells, obtained from humans. In some embodiments, libraries, such as single-chain TCR (scTv) libraries, can be assembled from naïve Vα and Vβ libraries in which the amplified products are cloned or assembled to be separated by a linker. Depending on the source of the subject and cells, the libraries can be HLA allele-specific. Alternatively, in some embodiments, TCR libraries can be generated by mutagenesis or diversification of a parent or scaffold TCR molecule.

In some aspects, the encoded TCRs are subjected to directed evolution, such as by mutagenesis, e.g., of the α or β chain. In some aspects, particular residues within CDRs of the TCR are altered. In some embodiments, selected TCRs can be modified by affinity maturation. In some embodiments, antigen-specific T cells may be selected, such as by screening to assess CTL activity against the peptide. In some aspects, encoded TCRs, e.g. present on the antigen-specific T cells, may be selected, such as by binding activity, e.g., particular affinity or avidity for the antigen.

In some embodiments, the encoded TCR or antigen-binding portion thereof is one that has been modified or engineered. In some embodiments, directed evolution methods are used to generate TCRs with altered properties, such as with higher affinity for a specific MHC-peptide complex. In some embodiments, directed evolution is achieved by display methods including, but not limited to, yeast display (Holler et al. (2003) Nat Immunol, 4, 55-62; Holler et al. (2000) Proc Natl Acad Sci USA, 97, 5387-92), phage display (Li et al. (2005) Nat Biotechnol, 23, 349-54), or T cell display (Chervin et al. (2008) J Immunol Methods, 339, 175-84). In some embodiments, display approaches involve engineering, or modifying, a known, parent or reference TCR. For example, in some cases, a wild-type TCR can be used as a template for producing mutagenized TCRs in which in one or more residues of the CDRs are mutated, and mutants with an desired altered property, such as higher affinity for a desired target antigen, are selected.

In some embodiments, the antigen is a tumor antigen that can be a glioma-associated antigen, f-human chorionic gonadotropin, alphafetoprotein (AFP), B-cell maturation antigen (BCMA, BCM), B-cell activating factor receptor (BAFFR, BR3), and/or transmembrane activator and CAML interactor (TACI), Fc Receptor-like 5 (FCRL5, FcRH5), lectin-reactive AFP, thyroglobulin, RAGE-1, MN-CA IX, human telomerase reverse transcriptase, RU1, RU2 (AS), intestinal carboxyl esterase, mut hsp70-2, M-CSF, Melanin-A/MART-1, WT-1, S-100, MBP, CD63, MUC1 (e.g. MUC1-8), p53, Ras, cyclin B1, HER-2/neu, carcinoembryonic antigen (CEA), gp100, MAGE-A1, MAGE-A2, MAGE-A3, MAGE-A4, MAGE-A5, MAGE-A6, MAGE-A7, MAGE-A8, MAGE-A9, MAGE-A10, MAGE-A11, MAGE-A11, MAGE-B1, MAGE-B2, MAGE-B3, MAGE-B4, MAGE-C1, BAGE, GAGE-1, GAGE-2, p15, tyrosinase, tyrosinase-related protein 1 (TRP-1), tyrosinase-related protein 2 (TRP-2), 0-catenin, NY-ESO-1, LAGE-1a, PP1, MDM2, MDM4, EGVFvIII, Tax, SSX2, telomerase, TARP, pp65, CDK4, vimentin, 5100, eIF-4A1, IFN-inducible p78, and melanotransferrin (p97), Uroplakin II, prostate specific antigen (PSA), human kallikrein (huK2), prostate specific membrane antigen (PSM), and prostatic acid phosphatase (PAP), neutrophil elastase, ephrin B2, BA-46, beta-catenin, Bcr-abl, E2A-PRL, H4-RET, IGH-IGK, MYL-RAR, Caspase 8 or a B-Raf antigen. Other tumor antigens can include any derived from FRa, CD24, CD44, CD133, CD 166, epCAM, CA-125, HE4, Oval, estrogen receptor, progesterone receptor, uPA, PAI-1, CD19, CD20, CD22, ROR1, mesothelin, CD33/IL3Ra, c-Met, PSMA, Glycolipid F77, GD-2, insulin growth factor (IGF)-I, IGF-II and IGF-I receptor. Specific tumor-associated antigens or T cell epitopes are known (see e.g. van der Bruggen et al. (2013) Cancer Immun, available at www.cancerimmunity.org/peptide/; Cheever et al. (2009) Clin Cancer Res, 15, 5323-37).

In some embodiments, the antigen is a viral antigen. Many viral antigen targets have been identified and are known, including peptides derived from viral genomes in HIV, HTLV and other viruses (see e.g., Addo et al. (2007) PLoS ONE, 2, e321; Tsomides et al. (1994) J Exp Med, 180, 1283-93; Utz et al. (1996) J Virol, 70, 843-51). Exemplary viral antigens include, but are not limited to, an antigen from hepatitis A, hepatitis B (e.g., HBV core and surface antigens (HBVc, HBVs)), hepatitis C (HCV), Epstein-Barr virus (e.g. EBVA), human papillomavirus (HPV; e.g. E6 and E7), human immunodeficiency type-1 virus (HIV1), Kaposi's sarcoma herpes virus (KSHV), human papilloma virus (HPV), influenza virus, Lassa virus, HTLN-1, HIN-1, HIN-II, CMN, EBN or HPN. In some embodiments, the target protein is a bacterial antigen or other pathogenic antigen, such as Mycobacterium tuberculosis (MT) antigens, trypanosome, e.g., Tiypansoma cruzi (T. cruzi), antigens such as surface antigen (TSA), or malaria antigens. Specific viral antigen or epitopes or other pathogenic antigens or T cell epitopes are known (see e.g., Addo et al. (2007) PLoS ONE, 2:e321; Anikeeva et al. (2009) Clin Immunol, 130:98-109).

In some embodiments, the antigen is an antigen derived from a virus associated with cancer, such as an oncogenic virus. For example, an oncogenic virus is one in which infection from certain viruses are known to lead to the development of different types of cancers, for example, hepatitis A, hepatitis B (e.g., HBV core and surface antigens (HBVc, HBVs)), hepatitis C (HCV), human papilloma virus (HPV), hepatitis viral infections, Epstein-Barr virus (EBV), human herpes virus 8 (HHV-8), human T-cell leukemia virus-1 (HTLV-1), human T-cell leukemia virus-2 (HTLV-2), or a cytomegalovirus (CMV) antigen.

In some embodiments, the viral antigen is an HPV antigen, which, in some cases, can lead to a greater risk of developing cervical cancer. In some embodiments, the antigen can be a HPV-16 antigen, and HPV-18 antigen, and HPV-31 antigen, an HPV-33 antigen or an HPV-35 antigen. In some embodiments, the viral antigen is an HPV-16 antigen (e.g., seroreactive regions of the E1, E2, E6 and/or E7 proteins of HPV-16, see e.g., U.S. Pat. No. 6,531,127) or an HPV-18 antigen (e.g., seroreactive regions of the L1 and/or L2 proteins of HPV-18, such as described in U.S. Pat. No. 5,840,306). In some embodiments, the viral antigen is an HPV-16 antigen that is from the E6 and/or E7 proteins of HPV-16. In some embodiments, the TCR is a TCR directed against an HPV-16 E6 or HPV-16 E7. In some embodiments, the TCR is a TCR described in, e.g., WO 2015/184228, WO 2015/009604 and WO 2015/009606.

In some embodiments, the viral antigen is a HBV or HCV antigen, which, in some cases, can lead to a greater risk of developing liver cancer than HBV or HCV negative subjects. For example, in some embodiments, the heterologous antigen is an HBV antigen, such as a hepatitis B core antigen or a hepatitis B envelope antigen (US2012/0308580).

In some embodiments, the viral antigen is an EBV antigen, which, in some cases, can lead to a greater risk for developing Burkitt's lymphoma, nasopharyngeal carcinoma and Hodgkin's disease than EBV negative subjects. For example, EBV is a human herpes virus that, in some cases, is found associated with numerous human tumors of diverse tissue origin. While primarily found as an asymptomatic infection, EBV-positive tumors can be characterized by active expression of viral gene products, such as EBNA-1, LMP-1 and LMP-2A. In some embodiments, the heterologous antigen is an EBV antigen that can include Epstein-Barr nuclear antigen (EBNA)-1, EBNA-2, EBNA-3A, EBNA-3B, EBNA-3C, EBNA-leader protein (EBNA-LP), latent membrane proteins LMP-1, LMP-2A and LMP-2B, EBV-EA, EBV-MA or EBV-VCA.

In some embodiments, the viral antigen is an HTLV-1 or HTLV-2 antigen, which, in some cases, can lead to a greater risk for developing T-cell leukemia than HTLV-1 or HTLV-2 negative subjects. For example, in some embodiments, the heterologous antigen is an HTLV-antigen, such as TAX.

In some embodiments, the viral antigen is a HHV-8 antigen, which, in some cases, can lead to a greater risk for developing Kaposi's sarcoma than HHV-8 negative subjects. In some embodiments, the heterologous antigen is a CMV antigen, such as pp65 or pp64 (see U.S. Pat. No. 8,361,473).

In some embodiments, the antigen is an autoantigen, such as an antigen of a polypeptide associated with an autoimmune disease or disorder. In some embodiments, the autoimmune disease or disorder can be multiple sclerosis (MS), rheumatoid arthritis (RA), Sjogren syndrome, scleroderma, polymyositis, dermatomyositis, systemic lupus erythematosus, juvenile rheumatoid arthritis, ankylosing spondylitis, myasthenia gravis (MG), bullous pemphigoid (antibodies to basement membrane at dermal-epidermal junction), pemphigus (antibodies to mucopolysaccharide protein complex or intracellular cement substance), glomerulonephritis (antibodies to glomerular basement membrane), Goodpasture's syndrome, autoimmune hemolytic anemia (antibodies to erythrocytes), Hashimoto's disease (antibodies to thyroid), pernicious anemia (antibodies to intrinsic factor), idiopathic thrombocytopenic purpura (antibodies to platelets), Grave's disease, or Addison's disease (antibodies to thyroglobulin). In some embodiments, the autoantigen, such as an autoantigen associated with one of the foregoing autoimmune disease, can be collagen, such as type II collagen, mycobacterial heat shock protein, thyroglobulin, acetyl choline receptor (AcHR), myelin basic protein (MBP) or proteolipid protein (PLP). Specific autoimmune associated epitopes or antigens are known (see e.g., Bulek et al. (2012) Nat Immunol, 13:283-9; Harkiolaki et al. (2009) Immunity, 30:348-57; Skowera et al. (2008) J Clin Invest, 1(18): 3390-402).

In some embodiments, peptides of a target polypeptide for use in producing or generating a TCR of interest are known or can be readily identified. In some embodiments, peptides suitable for use in generating TCRs or antigen-binding portions can be determined based on the presence of an HLA-restricted motif in a target polypeptide of interest, such as a target polypeptide described below. In some embodiments, peptides are identified using available computer prediction models. In some examples, HLA-A0201-binding motifs and the cleavage sites for proteasomes and immune-proteasomes using computer prediction models are known. In some embodiments, for predicting MHC class I binding sites, such models include, but are not limited to, ProPredl (Singh and Raghava (2001) Bioinformatics 17(12):1236-1237, and SYFPEITHI (see Schuler et al. (2007) Immunoinformatics Methods in Molecular Biology, 409(1): 75-93 2007). In some embodiments, the MHC-restricted epitope is HLA-A0201, which is expressed in approximately 39-46% of all Caucasians and therefore, represents a suitable choice of MHC antigen for use preparing a TCR or other MHC-peptide binding molecule.

In some embodiments, the TCR or antigen binding portion thereof may be a recombinantly produced natural protein or mutated form thereof in which one or more property, such as binding characteristic, has been altered. In some embodiments, a TCR may be derived from one of various animal species, such as human, mouse, rat, or other mammal. A TCR may be cell-bound or in soluble form. In some embodiments, for purposes of the provided methods, the TCR is in cell-bound form expressed on the surface of a cell.

In some embodiments, the encoded recombinant TCR is a full-length TCR. In some embodiments, the recombinant TCR is an antigen-binding portion. In some embodiments, the TCR is a dimeric TCR (dTCR). In some embodiments, the TCR is a single-chain TCR (scTCR). In some embodiments, a dTCR or scTCR have the structures as described in, e.g., International Pat. App. Pub. No. WO 03/020763, WO 04/033685 and WO 2011/044186.

In some embodiments, the encoded recombinant TCR contains a sequence corresponding to the transmembrane sequence. In some embodiments, the TCR does contain a sequence corresponding to cytoplasmic sequences. In some embodiments, the TCR is capable of forming a TCR complex with CD3. In some embodiments, any of the recombinant TCRs, including a dTCR or scTCR, can be linked to signaling domains that yield an active TCR on the surface of a T cell. In some embodiments, the recombinant TCR is expressed on the surface of cells. In some embodiments of the dTCR or scTCR containing introduced or engineered inter-chain disulfide bonds, the native disulfide bonds are not present.

In certain embodiments, the encoded TCR contains one or more modifications(s) to introduce one or more cysteine residues that are capable of forming one or more non-native disulfide bridges between the TCRα chain and TCRβ chain. In some embodiments, the encoded TCR contains a TCRα chain or a portion thereof containing a TCRα constant domain containing one or more cysteine residues capable of forming a non-native disulfide bond with a TCRβ chain. In some embodiments, the transgene encodes a TCRβ chain or a portion thereof containing a TCRβ constant domain containing one or more cysteine residues capable of forming a non-native disulfide bond with a TCRα chain. In some embodiments, the encoded TCR comprises a TCRα and/or a TCRβ chain and/or a TCRα and/or a TCRβ chain constant domains containing one or more modifications to introduce one or more disulfide bonds. In some embodiments, the transgene encodes a TCRα and/or a TCRβ chain and/or a TCRα and/or a TCRβ with one or more modifications to remove or prevent a native disulfide bond, e.g., between the TCRα encoded by the transgene and the endogenous TCRβ chain, or between the TCRβ encoded by the transgene and the endogenous TCRα chain. In some embodiments, one or more native cysteines that form and/or are capable of forming a native inter-chain disulfide bond are substituted to another residue, e.g., serine or alanine. In some embodiments, the cysteine is introduced at one or more of residue Thr48, Thr45, Tyr10, Thr45, and Ser15 with reference to numbering of a TCRα constant domain. In certain embodiments, cysteines can be introduced at residue Ser57, Ser77, Ser17, Asp59, of Glu15 of the TCRβ chain constant domain. Exemplary non-native disulfide bonds of a TCR are described in published International PCT No. WO2006/000830, WO 2006/037960 and Kuball et al. (2007) Blood, 109:2331-2338. In some embodiments, cysteines can be introduced or substituted at a residue corresponding to Thr48 of the Cα chain and Ser57 of the Cβ chain, at residue Thr45 of the Cα chain and Ser77 of the CO chain, at residue Tyr10 of the Cα chain and Ser17 of the Cβ chain, at residue Thr45 of the Cα chain and Asp59 of the Cβ chain and/or at residue Ser15 of the Cα chain and Glu15 of the Cβ chain. In some embodiments, any of the cysteine mutations can be made at a corresponding position in another sequence, for example, in a human or mouse Cα and Cβ sequence described above. The term “corresponding” with reference to positions of a protein, such as recitation that amino acid positions “correspond to” amino acid positions in an exemplary Cα and Cβ refers to amino acid positions identified upon alignment with the disclosed sequence based on structural sequence alignment or using a standard alignment algorithm, such as the GAP algorithm.

In some embodiments, the one or more of the native cysteines forming a native inter-chain disulfide bonds are substituted to another residue, such as to a serine or alanine. In some embodiments, an introduced or engineered disulfide bond can be formed by mutating non-cysteine residues on the first and second segments to cysteine. Exemplary non-native disulfide bonds of a TCR are described in published International PCT No. WO2006/000830.

In some embodiments, the encoded recombinant TCR is a dimeric TCR (dTCR). In some embodiments, the dTCR contains a first polypeptide wherein a sequence corresponding to a TCRα chain variable region sequence is fused to the N terminus of a sequence corresponding to a TCRα chain constant region extracellular sequence, and a second polypeptide wherein a sequence corresponding to a TCRβ chain variable region sequence is fused to the N terminus a sequence corresponding to a TCRβ chain constant region extracellular sequence, the first and second polypeptides being linked by a disulfide bond. In some embodiments, the bond can correspond to the native inter-chain disulfide bond present in native dimeric αβ TCRs. In some embodiments, the inter-chain disulfide bonds are not present in a native TCR. For example, in some embodiments, one or more cysteines can be incorporated into the constant region extracellular sequences of dTCR polypeptide pair. In some cases, both a native and a non-native disulfide bond may be desirable. In some embodiments, the TCR contains a transmembrane sequence to anchor to the membrane.

In some embodiments, the dTCR contains a TCRα chain containing a variable α domain, a constant α domain and a first dimerization motif attached to the C-terminus of the constant α domain, and a TCRβ chain comprising a variable β domain, a constant β domain and a first dimerization motif attached to the C-terminus of the constant β domain, wherein the first and second dimerization motifs interact to form a covalent bond between an amino acid in the first dimerization motif and an amino acid in the second dimerization motif linking the TCRα chain and TCRβ chain together.

In some embodiments, the encoded recombinant TCR is a single-chain TCR (scTCR or scTv). Typically, a scTCR can be generated using known methods, See e.g., Soo Hoo, W. F. et al. PNAS (USA) 89, 4759 (1992); Wulfing, C. and Pluckthun, A., J. Mol. Biol. 242, 655 (1994); Kurucz, I. et al. PNAS (USA) 90 3830 (1993); International Pat. App. Pub. Nos. WO 96/13593, WO 96/18105, WO 99/60120, WO 99/18129, WO 03/020763, WO 2011/044186; and Schlueter, C. J. et al. J. Mol. Biol. 256, 859 (1996). In some embodiments, the scTCR contains an introduced non-native disulfide inter-chain bond to facilitate the association of the TCR chains (see e.g. International Pat. App. Pub. No. WO 03/020763). In some embodiments, the scTCR is a non-disulfide linked truncated TCR in which heterologous leucine zippers fused to the C-termini thereof facilitate chain association (see e.g. International Pat. App. Pub. No. WO 99/60120). In some embodiments, the scTCR contains a TCRα variable domain covalently linked to a TCRβ variable domain via a peptide linker (see e.g., International Pat. App. Pub. No. WO 99/18129).

In some embodiments, the scTCR contains a first segment constituted by an amino acid sequence corresponding to a TCRα chain variable region, a second segment constituted by an amino acid sequence corresponding to a TCRβ chain variable region sequence fused to the N terminus of an amino acid sequence corresponding to a TCRβ chain constant domain extracellular sequence, and a linker sequence linking the C terminus of the first segment to the N terminus of the second segment. In some embodiments, the scTCR contains a first segment constituted by an α chain variable region sequence fused to the N terminus of an α chain extracellular constant domain sequence, and a second segment constituted by a β chain variable region sequence fused to the N terminus of a sequence β chain extracellular constant and transmembrane sequence, and, optionally, a linker sequence linking the C terminus of the first segment to the N terminus of the second segment. In some embodiments, the scTCR contains a first segment constituted by a TCRβ chain variable region sequence fused to the N terminus of a β chain extracellular constant domain sequence, and a second segment constituted by an α chain variable region sequence fused to the N terminus of a sequence α chain extracellular constant and transmembrane sequence, and, optionally, a linker sequence linking the C terminus of the first segment to the N terminus of the second segment.

In some embodiments, the linker of the scTCRs that links the first and second TCR segments can be any linker capable of forming a single polypeptide strand, while retaining TCR binding specificity. In some embodiments, the linker sequence may, for example, have the formula -P-AA-P- wherein P is proline and AA represents an amino acid sequence wherein the amino acids are glycine and serine. In some embodiments, the first and second segments are paired so that the variable region sequences thereof are orientated for such binding. Hence, in some cases, the linker has a sufficient length to span the distance between the C terminus of the first segment and the N terminus of the second segment, or vice versa, but is not too long to block or reduces bonding of the scTCR to the target ligand. In some embodiments, the linker can contain from or from about 10 to 45 amino acids, such as 10 to 30 amino acids or 26 to 41 amino acids residues, for example 29, 30, 31 or 32 amino acids. In some embodiments, the linker has the formula -PGGG-(SGGGG)₅-P- wherein P is proline, G is glycine and S is serine (SEQ ID NO:22). In some embodiments, the linker has the sequence

(SEQ ID NO: 23)
GSADDAKKDAAKKDGKS

In some embodiments, the scTCR contains a covalent disulfide bond linking a residue of the immunoglobulin region of the constant domain of the α chain to a residue of the immunoglobulin region of the constant domain of the β chain. In some embodiments, the interchain disulfide bond in a native TCR is not present. For example, in some embodiments, one or more cysteines can be incorporated into the constant region extracellular sequences of the first and second segments of the scTCR polypeptide. In some cases, both a native and a non-native disulfide bond may be desirable.

In some embodiments, the encoded TCR or antigen-binding fragment thereof exhibits an affinity with an equilibrium dissociation constant (K_{D}) for a target antigen of between or between about 10⁻⁵ and 10⁻¹² M and all individual values and ranges therein. In some embodiments, the target antigen is an MHC-peptide complex or ligand.

### C. Cells and Preparation of Cells for Genetic Engineering

In some embodiments, provided are engineered cells, e.g., genetically engineered or modified cells, and methods of engineering cells, including genetically engineered cells comprising a modified TGFBR2 locus that comprises a transgene sequence encoding a recombinant receptor or a portion thereof. In some embodiments, polynucleotides, e.g., template polynucleotides, such as any of the template polynucleotides described herein, such as in Section I.B.2, containing nucleic acid sequences encoding a recombinant receptor or a portion thereof and/or additional molecule(s), are introduced into one a cell for engineering, e.g., according to the methods of engineering described herein. In some aspects, the modified TGFBR2 locus of the engineered cell include those described in Section III.A herein.

In some aspects, the transgene sequences (exogenous or heterologous nucleic acid sequences) in the polynucleotides and/or portions thereof are heterologous, i.e., normally not present in a cell or sample obtained from the cell, such as one obtained from another organism or cell, which for example, is not ordinarily found in the cell being engineered and/or an organism from which such cell is derived. In some embodiments, the nucleic acid sequences are not naturally occurring, such as a nucleic acid sequences not found in nature or is modified from a nucleic acid sequence found in nature, including one comprising chimeric combinations of nucleic acids encoding various domains from multiple different cell types.

In some aspects, provided are method of producing a genetically engineered T cell, the method involving introducing any of the provided polynucleotides, e.g., described herein in Section I.B.2, into a T cell comprising a genetic disruption at a TGFBR2 locus. In some aspects, the genetic disruption is introduced by any agents or methods for introducing a targeted genetic disruption, including any described herein, such as in Section I.A. In some aspects, the method produces a modified TGFBR2 locus, said modified TGFBR2 locus comprising a nucleic acid sequence encoding the recombinant receptor. In some aspects, provided are method of producing a genetically engineered T cell that involves introducing, into a T cell, one or more agent(s) capable of inducing a genetic disruption at a target site within an endogenous TGFBR2 locus of the T cell; and introducing any of the provided polynucleotides, e.g., described herein in Section I.B.2, into a T cell comprising a genetic disruption at a TGFBR2 locus, wherein the method produces a modified TGFBR2 locus, said modified TGFBR2 locus comprising a nucleic acid sequence encoding the recombinant receptor, such as a CAR or a TCR. In some embodiments, the nucleic acid sequence comprises a transgene sequence encoding the recombinant receptor or a portion thereof, and the transgene sequence is targeted for integration within the endogenous TGFBR2 locus via homology directed repair (HDR).

In some embodiments, provided are methods of producing a genetically engineered T cell that involves introducing, into a T cell, a polynucleotide comprising a nucleic acid sequence encoding a recombinant receptor or a portion thereof, said T cell having a genetic disruption within a TGFBR2 locus of the T cell, wherein the nucleic acid sequence encoding the recombinant receptor or a portion thereof is targeted for integration within the endogenous TGFBR2 locus via homology directed repair (HDR). In some embodiments, the method produces a modified TGFBR2 locus, said modified TGFBR2 locus comprising a nucleic acid sequence encoding a recombinant receptor. In some embodiments, the nucleic acid sequence comprises a transgene sequence encoding the recombinant receptor or a portion thereof, such as any described herein, for example, in Section I.B.2. In some embodiments, upon performance of the methods, the expression of the endogenous TGFBRII is reduced or eliminated, or a non-functional and/or partial sequence of TGFBRII is expressed. In some embodiments, upon performance of the methods, a dominant negative (DN) form of TGFBRII is expressed.

The cells generally are eukaryotic cells, such as mammalian cells, and typically are human cells. In some embodiments, the cells are derived from the blood, bone marrow, lymph, or lymphoid organs, are cells of the immune system, such as cells of the innate or adaptive immunity, e.g., myeloid or lymphoid cells, including lymphocytes, typically T cells and/or NK cells. Other exemplary cells include stem cells, such as multipotent and pluripotent stem cells, including induced pluripotent stem cells (iPSCs). The cells typically are primary cells, such as those isolated directly from a subject and/or isolated from a subject and frozen. In some embodiments, the cells include one or more subsets of T cells or other cell types, such as whole T cell populations, CD4+ cells, CD8+ cells, and subpopulations thereof, such as those defined by function, activation state, maturity, potential for differentiation, expansion, recirculation, localization, and/or persistence capacities, antigen-specificity, type of antigen receptor, presence in a particular organ or compartment, marker or cytokine secretion profile, and/or degree of differentiation. With reference to the subject to be treated, the cells may be allogeneic and/or autologous. Among the methods include off-the-shelf methods. In some aspects, such as for off-the-shelf technologies, the cells are pluripotent and/or multipotent, such as stem cells, such as iPSCs. In some embodiments, the methods include isolating cells from the subject, preparing, processing, culturing, and/or engineering them, and re-introducing them into the same subject, before or after cryopreservation.

Among the sub-types and subpopulations of T cells and/or of CD4+ and/or of CD8+ T cells are naïve T (T_{N}) cells, effector T cells (T_{EFF}), memory T cells and sub-types thereof, such as stem cell memory T (T_{SCM}), central memory T (T_{CM}), effector memory T (T_{EM}), or terminally differentiated effector memory T cells, tumor-infiltrating lymphocytes (TIL), immature T cells, mature T cells, helper T cells, cytotoxic T cells, mucosa-associated invariant T (MAIT) cells, naturally occurring and adaptive regulatory T (Treg) cells, helper T cells, such as TH1 cells, TH2 cells, TH3 cells, TH17 cells, TH9 cells, TH22 cells, follicular helper T cells, alpha/beta T cells, and delta/gamma T cells.

In some embodiments, the cells are natural killer (NK) cells. In some embodiments, the cells are monocytes or granulocytes, e.g., myeloid cells, macrophages, neutrophils, dendritic cells, mast cells, eosinophils, and/or basophils. In some embodiments, the cells include one or more nucleic acids introduced via genetic engineering, and thereby express recombinant or genetically engineered products of such nucleic acids. In some embodiments, the nucleic acids are heterologous, i.e., normally not present in a cell or sample obtained from the cell, such as one obtained from another organism or cell, which for example, is not ordinarily found in the cell being engineered and/or an organism from which such cell is derived. In some embodiments, the nucleic acids are not naturally occurring, such as a nucleic acid not found in nature, including one comprising chimeric combinations of nucleic acids encoding various domains from multiple different cell types.

In some embodiments, preparation of the engineered cells includes one or more culture and/or preparation steps. The cells for introduction of the nucleic acid encoding the transgenic receptor such as the CAR, may be isolated from a sample, such as a biological sample, e.g., one obtained from or derived from a subject. In some embodiments, the subject from which the cell is isolated is one having the disease or condition or in need of a cell therapy or to which cell therapy will be administered. The subject in some embodiments is a human in need of a particular therapeutic intervention, such as the adoptive cell therapy for which cells are being isolated, processed, and/or engineered.

Accordingly, the cells in some embodiments are primary cells, e.g., primary human cells. The samples include tissue, fluid, and other samples taken directly from the subject, as well as samples resulting from one or more processing steps, such as separation, centrifugation, genetic engineering (e.g. transduction with viral vector), washing, and/or incubation. The biological sample can be a sample obtained directly from a biological source or a sample that is processed. Biological samples include, but are not limited to, body fluids, such as blood, plasma, serum, cerebrospinal fluid, synovial fluid, urine and sweat, tissue and organ samples, including processed samples derived therefrom.

In some aspects, the sample from which the cells are derived or isolated is blood or a blood-derived sample, or is or is derived from an apheresis or leukapheresis product. Exemplary samples include whole blood, peripheral blood mononuclear cells (PBMCs), leukocytes, bone marrow, thymus, tissue biopsy, tumor, leukemia, lymphoma, lymph node, gut associated lymphoid tissue, mucosa associated lymphoid tissue, spleen, other lymphoid tissues, liver, lung, stomach, intestine, colon, kidney, pancreas, breast, bone, prostate, cervix, testes, ovaries, tonsil, or other organ, and/or cells derived therefrom. Samples include, in the context of cell therapy, e.g., adoptive cell therapy, samples from autologous and allogeneic sources.

In some embodiments, the cells are derived from cell lines, e.g., T cell lines. The cells in some embodiments are obtained from a xenogeneic source, for example, from mouse, rat, non-human primate, and pig.

In some embodiments, isolation of the cells includes one or more preparation and/or non-affinity based cell separation steps. In some examples, cells are washed, centrifuged, and/or incubated in the presence of one or more reagents, for example, to remove unwanted components, enrich for desired components, lyse or remove cells sensitive to particular reagents. In some examples, cells are separated based on one or more property, such as density, adherent properties, size, sensitivity and/or resistance to particular components.

In some examples, cells from the circulating blood of a subject are obtained, e.g., by apheresis or leukapheresis. The samples, in some aspects, contain lymphocytes, including T cells, monocytes, granulocytes, B cells, other nucleated white blood cells, red blood cells, and/or platelets, and in some aspects contains cells other than red blood cells and platelets.

In some embodiments, the blood cells collected from the subject are washed, e.g., to remove the plasma fraction and to place the cells in an appropriate buffer or media for subsequent processing steps. In some embodiments, the cells are washed with phosphate buffered saline (PBS). In some embodiments, the wash solution lacks calcium and/or magnesium and/or many or all divalent cations. In some aspects, a washing step is accomplished a semi-automated “flow-through” centrifuge (for example, the Cobe 2991 cell processor, Baxter) according to the manufacturer's instructions. In some aspects, a washing step is accomplished by tangential flow filtration (TFF) according to the manufacturer's instructions. In some embodiments, the cells are resuspended in a variety of biocompatible buffers after washing, such as, for example, Ca⁺⁺/Mg⁺⁺ free PBS. In certain embodiments, components of a blood cell sample are removed and the cells directly resuspended in culture media.

In some embodiments, the methods include density-based cell separation methods, such as the preparation of white blood cells from peripheral blood by lysing the red blood cells and centrifugation through a Percoll or Ficoll gradient.

In some embodiments, the isolation methods include the separation of different cell types based on the expression or presence in the cell of one or more specific molecules, such as surface markers, e.g., surface proteins, intracellular markers, or nucleic acid. In some embodiments, any known method for separation based on such markers may be used. In some embodiments, the separation is affinity- or immunoaffinity-based separation. For example, the isolation in some aspects includes separation of cells and cell populations based on the cells' expression or expression level of one or more markers, typically cell surface markers, for example, by incubation with an antibody or binding partner that specifically binds to such markers, followed generally by washing steps and separation of cells having bound the antibody or binding partner, from those cells having not bound to the antibody or binding partner.

Such separation steps can be based on positive selection, in which the cells having bound the reagents are retained for further use, and/or negative selection, in which the cells having not bound to the antibody or binding partner are retained. In some examples, both fractions are retained for further use. In some aspects, negative selection can be particularly useful where no antibody is available that specifically identifies a cell type in a heterogeneous population, such that separation is best carried out based on markers expressed by cells other than the desired population.

The separation need not result in 100% enrichment or removal of a particular cell population or cells expressing a particular marker. For example, positive selection of or enrichment for cells of a particular type, such as those expressing a marker, refers to increasing the number or percentage of such cells, but need not result in a complete absence of cells not expressing the marker. Likewise, negative selection, removal, or depletion of cells of a particular type, such as those expressing a marker, refers to decreasing the number or percentage of such cells, but need not result in a complete removal of all such cells.

In some examples, multiple rounds of separation steps are carried out, where the positively or negatively selected fraction from one step is subjected to another separation step, such as a subsequent positive or negative selection. In some examples, a single separation step can deplete cells expressing multiple markers simultaneously, such as by incubating cells with a plurality of antibodies or binding partners, each specific for a marker targeted for negative selection. Likewise, multiple cell types can simultaneously be positively selected by incubating cells with a plurality of antibodies or binding partners expressed on the various cell types.

For example, in some aspects, specific subpopulations of T cells, such as cells positive or expressing high levels of one or more surface markers, e.g., CD28⁺, CD62L⁺, CCR7⁺, CD27⁺, CD127⁺, CD4⁺, CD8⁺, CD45RA⁺, and/or CD45RO⁺ T cells, are isolated by positive or negative selection techniques.

For example, CD3⁺, CD28⁺ T cells can be positively selected using anti-CD3/anti-CD28 conjugated magnetic beads (e.g., DYNABEADS® M-450 CD3/CD28 T Cell Expander).

In some embodiments, isolation is carried out by enrichment for a particular cell population by positive selection, or depletion of a particular cell population, by negative selection. In some embodiments, positive or negative selection is accomplished by incubating cells with one or more antibodies or other binding agent that specifically bind to one or more surface markers expressed or expressed (marker⁺) at a relatively higher level (marker^{high}) on the positively or negatively selected cells, respectively.

In some embodiments, T cells are separated from a PBMC sample by negative selection of markers expressed on non-T cells, such as B cells, monocytes, or other white blood cells, such as CD14. In some aspects, a CD4⁺ or CD8⁺ selection step is used to separate CD4⁺ helper and CD8⁺ cytotoxic T cells. Such CD4⁺ and CD8⁺ populations can be further sorted into sub-populations by positive or negative selection for markers expressed or expressed to a relatively higher degree on one or more naive, memory, and/or effector T cell subpopulations.

In some embodiments, CD8⁺ cells are further enriched for or depleted of naive, central memory, effector memory, and/or central memory stem cells, such as by positive or negative selection based on surface antigens associated with the respective subpopulation. In some embodiments, enrichment for central memory T (T_{CM}) cells is carried out to increase efficacy, such as to improve long-term survival, expansion, and/or engraftment following administration, which in some aspects is particularly robust in such sub-populations. See Terakura et al. (2012) Blood. 1:72-82; Wang et al. (2012) J Immunother. 35(9):689-701. In some embodiments, combining T_{CM}-enriched CD8⁺ T cells and CD4⁺ T cells further enhances efficacy.

In embodiments, memory T cells are present in both CD62L⁺ and CD62L⁻ subsets of CD8⁺ peripheral blood lymphocytes. PBMC can be enriched for or depleted of CD62L-CD8⁺ and/or CD62L⁺CD8⁺ fractions, such as using anti-CD8 and anti-CD62L antibodies.

In some embodiments, the enrichment for central memory T (T_{CM}) cells is based on positive or high surface expression of CD45RO, CD62L, CCR7, CD28, CD3, and/or CD127; in some aspects, it is based on negative selection for cells expressing or highly expressing CD45RA and/or granzyme B. In some aspects, isolation of a CD8⁺ population enriched for T_{CM} cells is carried out by depletion of cells expressing CD4, CD14, CD45RA, and positive selection or enrichment for cells expressing CD62L. In one aspect, enrichment for central memory T (T_{CM}) cells is carried out starting with a negative fraction of cells selected based on CD4 expression, which is subjected to a negative selection based on expression of CD14 and CD45RA, and a positive selection based on CD62L. Such selections in some aspects are carried out simultaneously and in other aspects are carried out sequentially, in either order. In some aspects, the same CD4 expression-based selection step used in preparing the CD8⁺ cell population or subpopulation, also is used to generate the CD4⁺ cell population or sub-population, such that both the positive and negative fractions from the CD4-based separation are retained and used in subsequent steps of the methods, optionally following one or more further positive or negative selection steps.

In a particular example, a sample of PBMCs or other white blood cell sample is subjected to selection of CD4⁺ cells, where both the negative and positive fractions are retained. The negative fraction then is subjected to negative selection based on expression of CD14 and CD45RA or CD19, and positive selection based on a marker characteristic of central memory T cells, such as CD62L or CCR7, where the positive and negative selections are carried out in either order.

CD4⁺ T helper cells are sorted into naïve, central memory, and effector cells by identifying cell populations that have cell surface antigens. CD4⁺ lymphocytes can be obtained by standard methods. In some embodiments, naive CD4⁺ T lymphocytes are CD45RO, CD45RA⁺, CD62L⁺, CD4⁺ T cells. In some embodiments, central memory CD4⁺ cells are CD62L⁺ and CD45RO⁺. In some embodiments, effector CD4⁺ cells are CD62L and CD45RO.

In one example, to enrich for CD4⁺ cells by negative selection, a monoclonal antibody cocktail typically includes antibodies to CD14, CD20, CD11b, CD16, HLA-DR, and CD8. In some embodiments, the antibody or binding partner is bound to a solid support or matrix, such as a magnetic bead or paramagnetic bead, to allow for separation of cells for positive and/or negative selection. For example, in some embodiments, the cells and cell populations are separated or isolated using immunomagnetic (or affinity magnetic) separation techniques (reviewed in Methods in Molecular Medicine, vol. 58: Metastasis Research Protocols, Vol. 2: Cell Behavior In Vitro and In Vivo, p 17-25 Edited by: S. A. Brooks and U. Schumacher © Humana Press Inc., Totowa, N.J.).

In some aspects, the sample or composition of cells to be separated is incubated with small, magnetizable or magnetically responsive material, such as magnetically responsive particles or microparticles, such as paramagnetic beads (e.g., such as Dynalbeads or MACS beads). The magnetically responsive material, e.g., particle, generally is directly or indirectly attached to a binding partner, e.g., an antibody, that specifically binds to a molecule, e.g., surface marker, present on the cell, cells, or population of cells that it is desired to separate, e.g., that it is desired to negatively or positively select.

In some embodiments, the magnetic particle or bead comprises a magnetically responsive material bound to a specific binding member, such as an antibody or other binding partner. There are many well-known magnetically responsive materials used in magnetic separation methods. Suitable magnetic particles include those described in Molday, U.S. Pat. No. 4,452,773, and in European Patent Specification EP 452342 B, which are hereby incorporated by reference. Colloidal sized particles, such as those described in Owen U.S. Pat. No. 4,795,698, and Liberti et al., U.S. Pat. No. 5,200,084 are other examples.

The incubation generally is carried out under conditions whereby the antibodies or binding partners, or molecules, such as secondary antibodies or other reagents, which specifically bind to such antibodies or binding partners, which are attached to the magnetic particle or bead, specifically bind to cell surface molecules if present on cells within the sample.

In some aspects, the sample is placed in a magnetic field, and those cells having magnetically responsive or magnetizable particles attached thereto will be attracted to the magnet and separated from the unlabeled cells. For positive selection, cells that are attracted to the magnet are retained; for negative selection, cells that are not attracted (unlabeled cells) are retained. In some aspects, a combination of positive and negative selection is performed during the same selection step, where the positive and negative fractions are retained and further processed or subject to further separation steps.

In certain embodiments, the magnetically responsive particles are coated in primary antibodies or other binding partners, secondary antibodies, lectins, enzymes, or streptavidin. In certain embodiments, the magnetic particles are attached to cells via a coating of primary antibodies specific for one or more markers. In certain embodiments, the cells, rather than the beads, are labeled with a primary antibody or binding partner, and then cell-type specific secondary antibody- or other binding partner (e.g., streptavidin)-coated magnetic particles, are added. In certain embodiments, streptavidin-coated magnetic particles are used in conjunction with biotinylated primary or secondary antibodies.

In some embodiments, the magnetically responsive particles are left attached to the cells that are to be subsequently incubated, cultured and/or engineered; in some aspects, the particles are left attached to the cells for administration to a patient. In some embodiments, the magnetizable or magnetically responsive particles are removed from the cells. Methods for removing magnetizable particles from cells are known and include, e.g., the use of competing non-labeled antibodies, and magnetizable particles or antibodies conjugated to cleavable linkers. In some embodiments, the magnetizable particles are biodegradable.

In some embodiments, the affinity-based selection is via magnetic-activated cell sorting (MACS) (Miltenyi Biotec, Auburn, Calif.). Magnetic Activated Cell Sorting (MACS) systems are capable of high-purity selection of cells having magnetized particles attached thereto. In certain embodiments, MACS operates in a mode wherein the non-target and target species are sequentially eluted after the application of the external magnetic field. That is, the cells attached to magnetized particles are held in place while the unattached species are eluted. Then, after this first elution step is completed, the species that were trapped in the magnetic field and were prevented from being eluted are freed in some manner such that they can be eluted and recovered. In certain embodiments, the non-target cells are labelled and depleted from the heterogeneous population of cells.

In certain embodiments, the isolation or separation is carried out using a system, device, or apparatus that carries out one or more of the isolation, cell preparation, separation, processing, incubation, culture, and/or formulation steps of the methods. In some aspects, the system is used to carry out each of these steps in a closed or sterile environment, for example, to minimize error, user handling and/or contamination. In one example, the system is a system as described in International Pat. App. Pub. No. WO2009/072003 or US 20110003380.

In some embodiments, the system or apparatus carries out one or more, e.g., all, of the isolation, processing, engineering, and formulation steps in an integrated or self-contained system, and/or in an automated or programmable fashion. In some aspects, the system or apparatus includes a computer and/or computer program in communication with the system or apparatus, which allows a user to program, control, assess the outcome of, and/or adjust various aspects of the processing, isolation, engineering, and formulation steps.

In some aspects, the separation and/or other steps is carried out using CliniMACS system (Miltenyi Biotec), for example, for automated separation of cells on a clinical-scale level in a closed and sterile system. Components can include an integrated microcomputer, magnetic separation unit, peristaltic pump, and various pinch valves. The integrated computer in some aspects controls all components of the instrument and directs the system to perform repeated procedures in a standardized sequence. The magnetic separation unit in some aspects includes a movable permanent magnet and a holder for the selection column. The peristaltic pump controls the flow rate throughout the tubing set and, together with the pinch valves, ensures the controlled flow of buffer through the system and continual suspension of cells.

The CliniMACS system in some aspects uses antibody-coupled magnetizable particles that are supplied in a sterile, non-pyrogenic solution. In some embodiments, after labelling of cells with magnetic particles the cells are washed to remove excess particles. A cell preparation bag is then connected to the tubing set, which in turn is connected to a bag containing buffer and a cell collection bag. The tubing set consists of pre-assembled sterile tubing, including a pre-column and a separation column, and are for single use only. After initiation of the separation program, the system automatically applies the cell sample onto the separation column. Labelled cells are retained within the column, while unlabeled cells are removed by a series of washing steps. In some embodiments, the cell populations for use with the methods described herein are unlabeled and are not retained in the column. In some embodiments, the cell populations for use with the methods described herein are labeled and are retained in the column. In some embodiments, the cell populations for use with the methods described herein are eluted from the column after removal of the magnetic field, and are collected within the cell collection bag.

In certain embodiments, separation and/or other steps are carried out using the CliniMACS Prodigy system (Miltenyi Biotec). The CliniMACS Prodigy system in some aspects is equipped with a cell processing unity that permits automated washing and fractionation of cells by centrifugation. The CliniMACS Prodigy system can also include an onboard camera and image recognition software that determines the optimal cell fractionation endpoint by discerning the macroscopic layers of the source cell product. For example, peripheral blood is automatically separated into erythrocytes, white blood cells and plasma layers. The CliniMACS Prodigy system can also include an integrated cell cultivation chamber which accomplishes cell culture protocols such as, e.g., cell differentiation and expansion, antigen loading, and long-term cell culture. Input ports can allow for the sterile removal and replenishment of media and cells can be monitored using an integrated microscope. See, e.g., Klebanoff et al. (2012) J Immunother. 35(9): 651-660, Terakura et al. (2012) Blood. 1:72-82, and Wang et al. (2012) J Immunother. 35(9):689-701.

In some embodiments, a cell population described herein is collected and enriched (or depleted) via flow cytometry, in which cells stained for multiple cell surface markers are carried in a fluidic stream. In some embodiments, a cell population described herein is collected and enriched (or depleted) via preparative scale (FACS)-sorting. In certain embodiments, a cell population described herein is collected and enriched (or depleted) by use of microelectromechanical systems (MEMS) chips in combination with a FACS-based detection system (see, e.g., WO 2010/033140, Cho et al. (2010) Lab Chip 10, 1567-1573; and Godin et al. (2008) J Biophoton. 1(5):355-376. In both cases, cells can be labeled with multiple markers, allowing for the isolation of well-defined T cell subsets at high purity.

In some embodiments, the antibodies or binding partners are labeled with one or more detectable marker, to facilitate separation for positive and/or negative selection. For example, separation may be based on binding to fluorescently labeled antibodies. In some examples, separation of cells based on binding of antibodies or other binding partners specific for one or more cell surface markers are carried in a fluidic stream, such as by fluorescence-activated cell sorting (FACS), including preparative scale (FACS) and/or microelectromechanical systems (MEMS) chips, e.g., in combination with a flow-cytometric detection system. Such methods allow for positive and negative selection based on multiple markers simultaneously.

In some embodiments, the preparation methods include steps for freezing, e.g., cryopreserving, the cells, either before or after isolation, incubation, and/or engineering. In some embodiments, the freeze and subsequent thaw step removes granulocytes and, to some extent, monocytes in the cell population. In some embodiments, the cells are suspended in a freezing solution, e.g., following a washing step to remove plasma and platelets. Any of a variety of known freezing solutions and parameters in some aspects may be used. One example involves using PBS containing 20% DMSO and 8% human serum albumin (HSA), or other suitable cell freezing media. This is then diluted 1:1 with media so that the final concentration of DMSO and HSA are 10% and 4%, respectively. The cells are generally then frozen to −80° C. at a rate of 1° per minute and stored in the vapor phase of a liquid nitrogen storage tank.

In some embodiments, the cells are incubated and/or cultured prior to or in connection with genetic engineering. The incubation steps can include culture, cultivation, stimulation, activation, and/or propagation. The incubation and/or engineering may be carried out in a culture vessel, such as a unit, chamber, well, column, tube, tubing set, valve, vial, culture dish, bag, or other container for culture or cultivating cells. In some embodiments, the compositions or cells are incubated in the presence of stimulating conditions or a stimulatory agent. Such conditions include those designed to induce proliferation, expansion, activation, and/or survival of cells in the population, to mimic antigen exposure, and/or to prime the cells for genetic engineering, such as for the introduction of a recombinant antigen receptor.

The conditions can include one or more of particular media, temperature, oxygen content, carbon dioxide content, time, agents, e.g., nutrients, amino acids, antibiotics, ions, and/or stimulatory factors, such as cytokines, chemokines, antigens, binding partners, fusion proteins, recombinant soluble receptors, and any other agents designed to activate the cells.

In some embodiments, the stimulating conditions or agents include one or more agent, e.g., ligand, which is capable of stimulating or activating an intracellular signaling domain of a TCR complex. In some aspects, the agent turns on or initiates TCR/CD3 intracellular signaling cascade in a T cell. Such agents can include antibodies, such as those specific for a TCR, e.g. anti-CD3. In some embodiments, the stimulating conditions include one or more agent, e.g. ligand, which is capable of stimulating a costimulatory receptor, e.g., anti-CD28. In some embodiments, such agents and/or ligands may be, bound to solid support such as a bead, and/or one or more cytokines. Optionally, the expansion method may further comprise the step of adding anti-CD3 and/or anti CD28 antibody to the culture medium (e.g., at a concentration of at least about 0.5 ng/mL). In some embodiments, the stimulating agents include IL-2, IL-15 and/or IL-7. In some aspects, the IL-2 concentration is at least about 10 units/mL.

In some aspects, incubation is carried out in accordance with techniques such as those described in U.S. Pat. No. 6,040,177, Klebanoff et al. (2012) J Immunother. 35(9): 651-660, Terakura et al. (2012) Blood. 1:72-82, and/or Wang et al. (2012) J Immunother. 35(9):689-701.

In some embodiments, the T cells are expanded by adding to a culture-initiating composition feeder cells, such as non-dividing peripheral blood mononuclear cells (PBMC), (e.g., such that the resulting population of cells contains at least about 5, 10, 20, or 40 or more PBMC feeder cells for each T lymphocyte in the initial population to be expanded); and incubating the culture (e.g. for a time sufficient to expand the numbers of T cells). In some aspects, the non-dividing feeder cells can comprise gamma-irradiated PBMC feeder cells. In some embodiments, the PBMC are irradiated with gamma rays in the range of about 3000 to 3600 rads to prevent cell division. In some aspects, the feeder cells are added to culture medium prior to the addition of the populations of T cells.

In some embodiments, the stimulating conditions include temperature suitable for the growth of human T lymphocytes, for example, at least about 25 degrees Celsius, generally at least about 30 degrees, and generally at or about 37 degrees Celsius. Optionally, the incubation may further comprise adding non-dividing EBV-transformed lymphoblastoid cells (LCL) as feeder cells. LCL can be irradiated with gamma rays in the range of about 6000 to 10,000 rads. The LCL feeder cells in some aspects is provided in any suitable amount, such as a ratio of LCL feeder cells to initial T lymphocytes of at least about 10:1.

In embodiments, antigen-specific T cells, such as antigen-specific CD4+ and/or CD8+ T cells, are obtained by stimulating naive or antigen specific T lymphocytes with antigen. For example, antigen-specific T cell lines or clones can be generated to cytomegalovirus antigens by isolating T cells from infected subjects and stimulating the cells in vitro with the same antigen.

Various methods for the introduction of genetically engineered components, e.g., agents for inducing a genetic disruption and/or nucleic acids encoding recombinant receptors, e.g., CARs or TCRs, are known and may be used with the provided methods and compositions. Exemplary methods include those for transfer of nucleic acids encoding the polypeptides or receptors, including via viral vectors, e.g., retroviral or lentiviral, non-viral vectors or transposons, e.g. Sleeping Beauty transposon system. Methods of gene transfer can include transduction, electroporation or other method that results into gene transfer into the cell, or any delivery methods described in Section I.A herein. Other approaches and vectors for transfer of the nucleic acids encoding the recombinant products are those described, e.g., in WO2014055668 and U.S. Pat. No. 7,446,190.

In some embodiments, recombinant nucleic acids are transferred into T cells via electroporation (see, e.g., Chicaybam et al, (2013) PLoS ONE 8(3): e60298 and Van Tedeloo et al. (2000) Gene Therapy 7(16): 1431-1437). In some embodiments, recombinant nucleic acids are transferred into T cells via transposition (see, e.g., Manuri et al. (2010) Hum Gene Ther 21(4): 427-437; Sharma et al. (2013) Molec Ther Nucl Acids 2, e74; and Huang et al. (2009) Methods Mol Biol 506: 115-126). Other methods of introducing and expressing genetic material in immune cells include calcium phosphate transfection (such as described in Current Protocols in Molecular Biology, John Wiley & Sons, New York. N.Y.), protoplast fusion, cationic liposome-mediated transfection; tungsten particle-facilitated microparticle bombardment (Johnston, Nature, 346: 776-777 (1990)); and strontium phosphate DNA co-precipitation (Brash et al., Mol. Cell Biol., 7: 2031-2034 (1987)).

In some embodiments, gene transfer is accomplished by first stimulating the cell, such as by combining it with a stimulus that induces a response such as proliferation, survival, and/or activation, e.g., as measured by expression of a cytokine or activation marker, followed by transduction of the activated cells, and expansion in culture to numbers sufficient for clinical applications.

In some contexts, it may be desired to safeguard against the potential that overexpression of a stimulatory factor (for example, a lymphokine or a cytokine) could potentially result in an unwanted outcome or lower efficacy in a subject, such as a factor associated with toxicity in a subject. Thus, in some contexts, the engineered cells include gene segments that cause the cells to be susceptible to negative selection in vivo, such as upon administration in adoptive immunotherapy. For example in some aspects, the cells are engineered so that they can be eliminated as a result of a change in the in vivo condition of the patient to which they are administered. The negative selectable phenotype may result from the insertion of a gene that confers sensitivity to an administered agent, for example, a compound. Negative selectable genes include the Herpes simplex virus type I thymidine kinase (HSV-I TK) gene (Wigler et al., Cell 11:223, 1977) which confers ganciclovir sensitivity; the cellular hypoxanthine phosphribosyltransferase (HPRT) gene, the cellular adenine phosphoribosyltransferase (APRT) gene, bacterial cytosine deaminase (Mullen et al., Proc. Natl. Acad. Sci. USA. 89:33 (1992)).

In some embodiments, the cells, e.g., T cells, may be engineered either during or after expansion. This engineering for the introduction of the gene of the desired polypeptide or receptor can be carried out with any suitable retroviral vector, for example. The genetically modified cell population can then be liberated from the initial stimulus (the CD3/CD28 stimulus, for example) and subsequently be stimulated with a second type of stimulus (e.g. via a de novo introduced receptor). This second type of stimulus may include an antigenic stimulus in form of a peptide/MHC molecule, the cognate (cross-linking) ligand of the genetically introduced receptor (e.g. natural ligand of a CAR) or any ligand (such as an antibody) that directly binds within the framework of the new receptor (e.g. by recognizing constant regions within the receptor). See, for example, Cheadle et al, “Chimeric antigen receptors for T-cell based therapy” Methods Mol Biol. 2012; 907:645-66 or Barrett et al., Chimeric Antigen Receptor Therapy for Cancer Annual Review of Medicine Vol. 65: 333-347 (2014).

Among additional nucleic acids, e.g., genes for introduction are those to improve the efficacy of therapy, such as by promoting viability and/or function of transferred cells; genes to provide a genetic marker for selection and/or evaluation of the cells, such as to assess in vivo survival or localization; genes to improve safety, for example, by making the cell susceptible to negative selection in vivo as described by Lupton S. D. et al., Mol. and Cell Biol., 11:6 (1991); and Riddell et al., Human Gene Therapy 3:319-338 (1992); see also the publications of PCT/US91/08442 and PCT/US94/05601 by Lupton et al. describing the use of bifunctional selectable fusion genes derived from fusing a dominant positive selectable marker with a negative selectable marker. See, e.g., Riddell et al., U.S. Pat. No. 6,040,177, at columns 14-17.

As described herein, in some embodiments, the cells are incubated and/or cultured prior to or in connection with genetic engineering. The incubation steps can include culture, cultivation, stimulation, activation, propagation and/or freezing for preservation, e.g. cryopreservation.

### D. Composition of Cells Expressing Recombinant Receptor

Also provided are plurality or populations of the engineered cells, compositions containing such cells and/or enriched for such cells. In some aspects, the provided engineered cells and/or composition of engineered cells include any described herein, e.g., comprising a modified TGFBR2 locus comprising a transgene sequence encoding a recombinant receptor or a portion thereof, and/or are produced by the methods described herein. In some aspects, the plurality or population of engineered cells contain any of the engineered cells described herein, e.g., in Section III.C herein. In some aspects, the provided cells and cell composition can be engineered using any of the methods described herein, e.g., using agent(s) or methods for introducing genetic disruption, for example, as described in Section I.A herein, and/or using polynucleotides, such as template polynucleotide descried herein, for example in Section I.B.2, via homology-directed repair (HDR). In some aspects, such cell population and/or compositions provided herein is or are comprised in a pharmaceutical composition or a composition for therapeutic uses or methods, for example, as described in Section V herein.

In some embodiments, the provided cell population and/or compositions containing engineered cells include a cell population that exhibits more improved, uniform, homogeneous and/or stable expression and/or antigen binding by the recombinant receptor, e.g., exhibit reduced coefficient of variation, compared to the expression and/or antigen binding of cell populations and/or compositions generated using other methods. In some embodiments, the cell population and/or compositions exhibit at least 100%, 95%, 90%, 80%, 70%, 60%, 50%, 40%, 30%, 20% or 10% lower coefficient of variation of expression of the recombinant receptor and/or antigen binding by the recombinant receptor compared to a respective population generated using other methods, e.g., random integration of sequences encoding the recombinant receptor. The coefficient of variation is defined as standard deviation of expression of the nucleic acid of interest (e.g., transgene sequences encoding a recombinant receptor or a portion thereof) within a population of cells, for example CD4+ and/or CD8+ T cells, divided by the mean of expression of the respective nucleic acid of interest in the respective population of cells. In some embodiments, the cell population and/or compositions exhibit a coefficient of variation that is lower than 0.70, 0.65, 0.60, 0.55, 0.50, 0.45, 0.40, 0.35 or 0.30 or less, when measured among CD4+ and/or CD8+ T cell populations that have been engineered using the methods provided herein.

In some embodiments, the provided cell population and/or compositions containing engineered cells include a cell population that exhibits minimal or reduced random integration of the transgene encoding a recombinant receptor or a portion thereof. In some aspects, random integration of transgene into the genome of the cell can result in adverse effects or cell death due to integration of the transgene into undesired location in the genome, e.g., into an essential gene or a gene critical in regulating the activity of the cell, and/or unregulated or uncontrolled expression of the receptor. In some aspects, random integration of the transgene is reduced by at least or greater than 50%, 60%, 70%, 80%, 90%, 91%, 92%, 93%, 94%, 95%, 96%, 97%, 98%, 99%, or more compared to cell populations generated using other methods.

In some embodiments, provided are cell population and/or compositions that include a plurality of engineered immune cells expressing a recombinant receptor, wherein the nucleic acid sequence encoding the recombinant receptor is present at the TGFBR2 locus, e.g., by integration of a transgene encoding recombinant receptor or a portion thereof at the TGFBR2 locus via homology directed repair (HDR). In some embodiments, at least or greater than 30%, 35%, 40%, 45%, 50%, 55%, 60%, 65%, 70%, 75%, 80%, or 90% of the cells in the composition and/or cells in the composition that contains a genetic disruption at the TGFBR2 locus comprise integration of the transgene encoding recombinant receptor or a portion thereof at the TGFBR2 locus.

In some embodiments, the provided compositions containing cells such as in which cells expressing the recombinant receptor make up at least 30%, 40%, 50%, 60%, 70%, 80%, 90%, 91%, 92%, 93%, 94%, 95%, 96%, 97%, 98%, 99%, or more of the total cells in the composition or cells of a certain type such as T cells or CD8+ or CD4+ cells. In some embodiments, the provided compositions containing cells such as in which cells expressing the recombinant receptor make up at least 30%, 40%, 50%, 60%, 70%, 80%, 90%, 91%, 92%, 93%, 94%, 95%, 96%, 97%, 98%, 99%, or more of the total cells in the composition that contains a genetic disruption at the TGFBR2 locus.

### IV. METHODS OF TREATMENT

Provided herein are methods of treatment, e.g., including administering any of the engineered cells or compositions containing the engineered cells described herein, for example, engineered cells comprising a modified TGFBR2 locus comprising a transgene encoding a recombinant receptor or a portion thereof. In some aspects, also provided are methods of administering any of the engineered cells or compositions containing engineered cells described herein to a subject, such as a subject that has a disease or disorder. The engineered cells expressing a recombinant receptor, such as a chimeric antigen receptor (CAR) or a T cell receptor (TCR), or compositions comprising the same, described herein are useful in a variety of therapeutic, diagnostic and prophylactic indications. For example, the engineered cells or compositions comprising the engineered cells are useful in treating a variety of diseases and disorders in a subject. Such methods and uses include therapeutic methods and uses, for example, involving administration of the engineered cells, or compositions containing the same, to a subject having a disease, condition, or disorder, such as a tumor or cancer. In some embodiments, the engineered cells or compositions comprising the same are administered in an effective amount to effect treatment of the disease or disorder. Uses include uses of the engineered cells or compositions in such methods and treatments, and in the preparation of a medicament in order to carry out such therapeutic methods. In some embodiments, the methods are carried out by administering the engineered cells, or compositions comprising the same, to the subject having or suspected of having the disease or condition. In some embodiments, the methods thereby treat the disease or condition or disorder in the subject. Also provided are therapeutic methods for administering the cells and compositions to subjects, e.g., patients.

Methods for administration of cells for adoptive cell therapy are known and may be used in connection with the provided methods and compositions. For example, adoptive T cell therapy methods are described, e.g., in US Pat. App. Pub. No. 2003/0170238 to Gruenberg et al; U.S. Pat. No. 4,690,915 to Rosenberg; Rosenberg (2011) Nat Rev Clin Oncol. 8(10):577-85). See, e.g., Themeli et al. (2013) Nat Biotechnol. 31(10): 928-933; Tsukahara et al. (2013) Biochem Biophys Res Commun 438(1): 84-9; Davila et al. (2013) PLoS ONE 8(4): e61338.

The disease or condition that is treated can be any in which expression of an antigen is associated with and/or involved in the etiology of a disease condition or disorder, e.g. causes, exacerbates or otherwise is involved in such disease, condition, or disorder. Exemplary diseases and conditions can include diseases or conditions associated with malignancy or transformation of cells (e.g. cancer), autoimmune or inflammatory disease, or an infectious disease, e.g. caused by a bacterial, viral or other pathogen. Exemplary antigens, which include antigens associated with various diseases and conditions that can be treated, are described herein. In particular embodiments, the chimeric antigen receptor or transgenic TCR specifically binds to an antigen associated with the disease or condition.

Among the diseases, conditions, and disorders are tumors, including solid tumors, hematologic malignancies, and melanomas, and including localized and metastatic tumors, infectious diseases, such as infection with a virus or other pathogen, e.g., HIV, HCV, HBV, CMV, HPV, and parasitic disease, and autoimmune and inflammatory diseases. In some embodiments, the disease, disorder or condition is a tumor, cancer, malignancy, neoplasm, or other proliferative disease or disorder. Such diseases include but are not limited to leukemia, lymphoma, e.g., acute myeloid (or myelogenous) leukemia (AML), chronic myeloid (or myelogenous) leukemia (CML), acute lymphocytic (or lymphoblastic) leukemia (ALL), chronic lymphocytic leukemia (CLL), hairy cell leukemia (HCL), small lymphocytic lymphoma (SLL), Mantle cell lymphoma (MCL), Marginal zone lymphoma, Burkitt lymphoma, Hodgkin lymphoma (HL), non-Hodgkin lymphoma (NHL), Anaplastic large cell lymphoma (ALCL), follicular lymphoma, refractory follicular lymphoma, diffuse large B-cell lymphoma (DLBCL) and multiple myeloma (MM). In some embodiments, disease or condition is a B cell malignancy selected from among acute lymphoblastic leukemia (ALL), adult ALL, chronic lymphoblastic leukemia (CLL), non-Hodgkin lymphoma (NHL), and Diffuse Large B-Cell Lymphoma (DLBCL). In some embodiments, the disease or condition is NHL and the NHL is selected from the group consisting of aggressive NHL, diffuse large B cell lymphoma (DLBCL), NOS (de novo and transformed from indolent), primary mediastinal large B cell lymphoma (PMBCL), T cell/histocyte-rich large B cell lymphoma (TCHRBCL), Burkitt's lymphoma, mantle cell lymphoma (MCL), and/or follicular lymphoma (FL), optionally, follicular lymphoma Grade 3B (FL3B).

In some embodiments, the disease or disorder is a multiple myeloma (MM). In some embodiments, administration of the provided cells, e.g., engineered cells with a modified TGFBR2 locus, can result in treatment of and/or amelioration of a disease or condition, such as a MM in the subject. In some embodiments, the subject has or is suspected of having a MM that is associated with expression of a tumor-associated antigen, such as a B cell maturation antigen (BCMA).

In some embodiments, the disease or disorder is a chronic lymphocytic leukemia (CLL). In some embodiments, administration of the provided cells, e.g., engineered cells with a modified TGFBR2 locus, can result in treatment of and/or amelioration of a disease or condition, such as a CLL in the subject. In some embodiments, the subject has or is suspected of having a CLL that is associated with expression of a tumor-associated antigen, such as a Receptor Tyrosine Kinase Like Orphan Receptor 1 (ROR1).

In some embodiments, the disease or disorder is a solid tumor, or a cancer associated with a non-hematological tumor. In some embodiments, the disease or disorder is a solid tumor, or a cancer associated with a solid tumor. In some embodiments, the disease or disorder is a pancreatic cancer, bladder cancer, colorectal cancer, breast cancer, prostate cancer, renal cancer, hepatocellular cancer, lung cancer, ovarian cancer, cervical cancer, pancreatic cancer, rectal cancer, thyroid cancer, uterine cancer, gastric cancer, esophageal cancer, head and neck cancer, melanoma, neuroendocrine cancers, CNS cancers, brain tumors, bone cancer, or soft tissue sarcoma. In some embodiments, the disease or disorder is a bladder, lung, brain, melanoma (e.g. small-cell lung, melanoma), breast, cervical, ovarian, colorectal, pancreatic, endometrial, esophageal, kidney, liver, prostate, skin, thyroid, or uterine cancers. In some embodiments, the disease or disorder is a pancreatic cancer, bladder cancer, colorectal cancer, breast cancer, prostate cancer, renal cancer, hepatocellular cancer, lung cancer, ovarian cancer, cervical cancer, pancreatic cancer, rectal cancer, thyroid cancer, uterine cancer, gastric cancer, esophageal cancer, head and neck cancer, melanoma, neuroendocrine cancers, CNS cancers, brain tumors, bone cancer, or soft tissue sarcoma.

In some embodiments, the disease or disorder is a non-small cell lung cancer (NSCLC). In some embodiments, administration of the provided cells, e.g., engineered cells with a modified TGFBR2 locus, can result in treatment of and/or amelioration of a disease or condition, such as a NSCLC in the subject. In some embodiments, the subject has or is suspected of having a NSCLC that is associated with expression of a tumor-associated antigen, such as a Receptor Tyrosine Kinase Like Orphan Receptor 1 (ROR1).

In some embodiments, the disease or disorder is a head and neck squamous cell carcinoma (HNSCC). In some embodiments, administration of the provided cells, e.g., engineered cells with a modified TGFBR2 locus, can result in treatment of and/or amelioration of a disease or condition, such as a HNSCC in the subject. In some embodiments, the subject has or is suspected of having a HNSCC that is associated with expression of a tumor-associated antigen, such as a human papilloma virus (HPV) 16 E6 or E7. In some embodiments, the disease or condition is an infectious disease or condition, such as, but not limited to, viral, retroviral, bacterial, and protozoal infections, immunodeficiency, Cytomegalovirus (CMV), Epstein-Barr virus (EBV), adenovirus, BK polyomavirus. In some embodiments, the disease or condition is an autoimmune or inflammatory disease or condition, such as arthritis, e.g., rheumatoid arthritis (RA), Type I diabetes, systemic lupus erythematosus (SLE), inflammatory bowel disease, psoriasis, scleroderma, autoimmune thyroid disease, Grave's disease, Crohn's disease, multiple sclerosis, asthma, and/or a disease or condition associated with transplant.

In some embodiments, the antigen associated with the disease or disorder is or includes αvβ6 integrin (avb6 integrin), B cell maturation antigen (BCMA), B7-H3, B7-H6, carbonic anhydrase 9 (CA9, also known as CAIX or G250), a cancer-testis antigen, cancer/testis antigen 1B (CTAG, also known as NY-ESO-1 and LAGE-2), carcinoembryonic antigen (CEA), a cyclin, cyclin A2, C—C Motif Chemokine Ligand 1 (CCL-1), CD19, CD20, CD22, CD23, CD24, CD30, CD33, CD38, CD44, CD44v6, CD44v7/8, CD123, CD133, CD138, CD171, chondroitin sulfate proteoglycan 4 (CSPG4), epidermal growth factor protein (EGFR), type III epidermal growth factor receptor mutation (EGFR vIII), epithelial glycoprotein 2 (EPG-2), epithelial glycoprotein 40 (EPG-40), ephrinB2, ephrin receptor A2 (EPHa2), estrogen receptor, Fc receptor like 5 (FCRL5; also known as Fc receptor homolog 5 or FCRH5), fetal acetylcholine receptor (fetal AchR), a folate binding protein (FBP), folate receptor alpha, ganglioside GD2, O-acetylated GD2 (OGD2), ganglioside GD3, glycoprotein 100 (gp100), glypican-3 (GPC3), G protein-coupled receptor class C group 5 member D (GPRC5D), Her2/neu (receptor tyrosine kinase erb-B2), Her3 (erb-B3), Her4 (erb-B4), erbB dimers, Human high molecular weight-melanoma-associated antigen (HMW-MAA), hepatitis B surface antigen, Human leukocyte antigen A1 (HLA-A1), Human leukocyte antigen A2 (HLA-A2), IL-22 receptor alpha (IL-22Rα), IL-13 receptor alpha 2 (IL-13Rα2), kinase insert domain receptor (kdr), kappa light chain, L1 cell adhesion molecule (L1-CAM), CE7 epitope of L1-CAM, Leucine Rich Repeat Containing 8 Family Member A (LRRC8A), Lewis Y, Melanoma-associated antigen (MAGE)-A1, MAGE-A3, MAGE-A6, MAGE-A10, mesothelin (MSLN), c-Met, murine cytomegalovirus (CMV), mucin 1 (MUC1), MUC16, natural killer group 2 member D (NKG2D) ligands, melan A (MART-1), neural cell adhesion molecule (NCAM), oncofetal antigen, Preferentially expressed antigen of melanoma (PRAME), progesterone receptor, a prostate specific antigen, prostate stem cell antigen (PSCA), prostate specific membrane antigen (PSMA), Receptor Tyrosine Kinase Like Orphan Receptor 1 (ROR1), survivin, Trophoblast glycoprotein (TPBG also known as 5T4), tumor-associated glycoprotein 72 (TAG72), Tyrosinase related protein 1 (TRP1, also known as TYRP1 or gp75), Tyrosinase related protein 2 (TRP2, also known as dopachrome tautomerase, dopachrome delta-isomerase or DCT), vascular endothelial growth factor receptor (VEGFR), vascular endothelial growth factor receptor 2 (VEGFR2), Wilms Tumor 1 (WT-1), a pathogen-specific or pathogen-expressed antigen, or an antigen associated with a universal tag, and/or biotinylated molecules, and/or molecules expressed by HIV, HCV, HBV or other pathogens. Antigens targeted by the receptors in some embodiments include antigens associated with a B cell malignancy, such as any of a number of known B cell marker. In some embodiments, the antigen is or includes CD20, CD19, CD22, ROR1, CD45, CD21, CD5, CD33, Igkappa, Iglambda, CD79a, CD79b or CD30.

In some embodiments, the antigen is or includes a pathogen-specific or pathogen-expressed antigen. In some embodiments, the antigen is a viral antigen (such as a viral antigen from HIV, HCV, HBV, etc.), bacterial antigens, and/or parasitic antigens.

In some aspects, the recombinant receptor, such as a CAR, specifically binds to an antigen associated with the disease or condition or expressed in cells of the environment of a lesion associated with the B cell malignancy. Antigens targeted by the receptors in some embodiments include antigens associated with a B cell malignancy, such as any of a number of known B cell marker. In some embodiments, the antigen targeted by the receptor is CD20, CD19, CD22, ROR1, CD45, CD21, CD5, CD33, Igkappa, Iglambda, CD79a, CD79b or CD30, or combinations thereof.

In some embodiments, the disease or condition is a myeloma, such as a multiple myeloma. In some aspects, the recombinant receptor, such as a CAR, specifically binds to an antigen associated with the disease or condition or expressed in cells of the environment of a lesion associated with the multiple myeloma. Antigens targeted by the receptors in some embodiments include antigens associated with multiple myeloma. In some aspects, the antigen, e.g., the second or additional antigen, such as the disease-specific antigen and/or related antigen, is expressed on multiple myeloma, such as B cell maturation antigen (BCMA), G protein-coupled receptor class C group 5 member D (GPRC5D), CD38 (cyclic ADP ribose hydrolase), CD138 (syndecan-1, syndecan, SYN-1), CS-1 (CS1, CD2 subset 1, CRACC, SLAMF7, CD319, and 19A24), BAFF-R, TACI and/or FcRH5. Other exemplary multiple myeloma antigens include CD56, TIM-3, CD33, CD123, CD44, CD20, CD40, CD74, CD200, EGFR, β2-Microglobulin, HM1.24, IGF-1R, IL-6R, TRAIL-R1, and the activin receptor type IIA (ActRIIA). See Benson and Byrd, J. Clin. Oncol. (2012) 30(16): 2013-15; Tao and Anderson, Bone Marrow Research (2011):924058; Chu et al., Leukemia (2013) 28(4):917-27; Garfall et al., Discov Med. (2014) 17(91):37-46. In some embodiments, the antigens include those present on lymphoid tumors, myeloma, AIDS-associated lymphoma, and/or post-transplant lymphoproliferations, such as CD38. Antibodies or antigen-binding fragments directed against such antigens are known and include, for example, those described in U.S. Pat. Nos. 8,153,765; 8,603,477, 8,008,450; U.S. Pub. No. US20120189622 or US20100260748; and/or International PCT Publication Nos. WO2006099875, WO2009080829 or WO2012092612 or WO2014210064. In some embodiments, such antibodies or antigen-binding fragments thereof (e.g. scFv) are contained in multispecific antibodies, multispecific chimeric receptors, such as multispecific CARs, and/or multispecific cells.

In some embodiments, the disease or disorder is associated with expression of G protein-coupled receptor class C group 5 member D (GPRC5D) and/or expression of B cell maturation antigen (BCMA).

In some embodiments, the disease or disorder is a B cell-related disorder. In some of any of the provided embodiments of the provided methods, the disease or disorder associated with BCMA is an autoimmune disease or disorder. In some of any of the provided embodiments of the provided methods, the autoimmune disease or disorder is systemic lupus erythematosus (SLE), lupus nephritis, inflammatory bowel disease, rheumatoid arthritis, ANCA associated vasculitis, idiopathic thrombocytopenia purpura (ITP), thrombotic thrombocytopenia purpura (TTP), autoimmune thrombocytopenia, Chagas' disease, Grave's disease, Wegener's granulomatosis, poly-arteritis nodosa, Sjogren's syndrome, pemphigus vulgaris, scleroderma, multiple sclerosis, psoriasis, IgA nephropathy, IgM polyneuropathies, vasculitis, diabetes mellitus, Reynaud's syndrome, anti-phospholipid syndrome, Goodpasture's disease, Kawasaki disease, autoimmune hemolytic anemia, myasthenia gravis, or progressive glomerulonephritis.

In some embodiments, the disease or disorder is a cancer. In some embodiments, the cancer is a GPRC5D-expressing cancer. In some embodiments, the cancer is a plasma cell malignancy and the plasma cell malignancy is multiple myeloma (MM) or plasmacytoma. In some embodiments, the cancer is multiple myeloma (MM). In some embodiments, the cancer is a relapsed/refractory multiple myeloma.

In some embodiments, the antigen is associated a virus, such as a human papilloma virus (HPV), and the disease or disorder is a cancer, such as a HNSCC. In some embodiments, the antigen is ROR1, and the disease or disorder is CLL. In some embodiments, the antigen is ROR1, and the disease or disorder is NSCLC.

In some embodiments, the antibody or an antigen-binding fragment (e.g. scFv or V_{H} domain) specifically recognizes an antigen, such as CD19, BCMA, GPRC5D or ROR1. In some embodiments, the antibody or antigen-binding fragment is derived from, or is a variant of, antibodies or antigen-binding fragment that specifically binds to CD19, BCMA, GPRC5D or ROR1.

In some embodiments, the cell therapy, e.g., adoptive T cell therapy, is carried out by autologous transfer, in which the cells are isolated and/or otherwise prepared from the subject who is to receive the cell therapy, or from a sample derived from such a subject. Thus, in some aspects, the cells are derived from a subject, e.g., patient, in need of a treatment and the cells, following isolation and processing are administered to the same subject.

In some embodiments, the cell therapy, e.g., adoptive T cell therapy, is carried out by allogeneic transfer, in which the cells are isolated and/or otherwise prepared from a subject other than a subject who is to receive or who ultimately receives the cell therapy, e.g., a first subject. In such embodiments, the cells then are administered to a different subject, e.g., a second subject, of the same species. In some embodiments, the first and second subjects are genetically identical. In some embodiments, the first and second subjects are genetically similar. In some embodiments, the second subject expresses the same HLA class or supertype as the first subject.

The cells can be administered by any suitable means, for example, by bolus infusion, by injection, e.g., intravenous or subcutaneous injections, intraocular injection, periocular injection, subretinal injection, intravitreal injection, trans-septal injection, subscleral injection, intrachoroidal injection, intracameral injection, subconjectval injection, subconjuntival injection, sub-Tenon's injection, retrobulbar injection, peribulbar injection, or posterior juxtascleral delivery. In some embodiments, they are administered by parenteral, intrapulmonary, and intranasal, and, if desired for local treatment, intralesional administration. Parenteral infusions include intramuscular, intravenous, intraarterial, intraperitoneal, or subcutaneous administration. In some embodiments, a given dose is administered by a single bolus administration of the cells. In some embodiments, it is administered by multiple bolus administrations of the cells, for example, over a period of no more than 3 days, or by continuous infusion administration of the cells. In some embodiments, administration of the cell dose or any additional therapies, e.g., the lymphodepleting therapy, intervention therapy and/or combination therapy, is carried out via outpatient delivery.

For the prevention or treatment of disease, the appropriate dosage may depend on the type of disease to be treated, the type of cells or recombinant receptors, the severity and course of the disease, whether the cells are administered for preventive or therapeutic purposes, previous therapy, the subject's clinical history and response to the cells, and the discretion of the attending physician. The compositions and cells are in some embodiments suitably administered to the subject at one time or over a series of treatments.

In some embodiments, the cells are administered as part of a combination treatment, such as simultaneously with or sequentially with, in any order, another therapeutic intervention, such as an antibody or engineered cell or receptor or agent, such as a cytotoxic or therapeutic agent. The cells in some embodiments are co-administered with one or more additional therapeutic agents or in connection with another therapeutic intervention, either simultaneously or sequentially in any order. In some contexts, the cells are co-administered with another therapy sufficiently close in time such that the cell populations enhance the effect of one or more additional therapeutic agents, or vice versa. In some embodiments, the cells are administered prior to the one or more additional therapeutic agents. In some embodiments, the cells are administered after the one or more additional therapeutic agents. In some embodiments, the one or more additional agents include a cytokine, such as IL-2, for example, to enhance persistence. In some embodiments, the methods comprise administration of a chemotherapeutic agent.

In some embodiments, the methods comprise administration of a chemotherapeutic agent, e.g., a conditioning chemotherapeutic agent, for example, to reduce tumor burden prior to the administration.

Preconditioning subjects with immunodepleting (e.g., lymphodepleting) therapies in some aspects can improve the effects of adoptive cell therapy (ACT).

Thus, in some embodiments, the methods include administering a preconditioning agent, such as a lymphodepleting or chemotherapeutic agent, such as cyclophosphamide, fludarabine, or combinations thereof, to a subject prior to the initiation of the cell therapy. For example, the subject may be administered a preconditioning agent at least 2 days prior, such as at least 3, 4, 5, 6, or 7 days prior, to the initiation of the cell therapy. In some embodiments, the subject is administered a preconditioning agent no more than 7 days prior, such as no more than 6, 5, 4, 3, or 2 days prior, to the initiation of the cell therapy.

In some embodiments, the subject is preconditioned with cyclophosphamide at a dose between or between about 20 mg/kg and 100 mg/kg, such as between or between about 40 mg/kg and 80 mg/kg. In some aspects, the subject is preconditioned with or with about 60 mg/kg of cyclophosphamide. In some embodiments, the cyclophosphamide can be administered in a single dose or can be administered in a plurality of doses, such as given daily, every other day or every three days. In some embodiments, the cyclophosphamide is administered once daily for one or two days. In some embodiments, where the lymphodepleting agent comprises cyclophosphamide, the subject is administered cyclophosphamide at a dose between or between about 100 mg/m² and 500 mg/m², such as between or between about 200 mg/m² and 400 mg/m², or 250 mg/m² and 350 mg/m², inclusive. In some instances, the subject is administered about 300 mg/m² of cyclophosphamide. In some embodiments, the cyclophosphamide can be administered in a single dose or can be administered in a plurality of doses, such as given daily, every other day or every three days. In some embodiments, cyclophosphamide is administered daily, such as for 1-5 days, for example, for 3 to 5 days. In some instances, the subject is administered about 300 mg/m² of cyclophosphamide, daily for 3 days, prior to initiation of the cell therapy.

In some embodiments, where the lymphodepleting agent comprises fludarabine, the subject is administered fludarabine at a dose between or between about 1 mg/m² and 100 mg/m², such as between or between about 10 mg/m² and 75 mg/m², 15 mg/m² and 50 mg/m², 20 mg/m² and 40 mg/m², or 24 mg/m² and 35 mg/m², inclusive. In some instances, the subject is administered about 30 mg/m² of fludarabine. In some embodiments, the fludarabine can be administered in a single dose or can be administered in a plurality of doses, such as given daily, every other day or every three days. In some embodiments, fludarabine is administered daily, such as for 1-5 days, for example, for 3 to 5 days. In some instances, the subject is administered about 30 mg/m² of fludarabine, daily for 3 days, prior to initiation of the cell therapy.

In some embodiments, the lymphodepleting agent comprises a combination of agents, such as a combination of cyclophosphamide and fludarabine. Thus, the combination of agents may include cyclophosphamide at any dose or administration schedule, such as those described herein, and fludarabine at any dose or administration schedule, such as those described herein. For example, in some aspects, the subject is administered 60 mg/kg (˜2 g/m²) of cyclophosphamide and 3 to 5 doses of 25 mg/m² fludarabine prior to the first or subsequent dose.

Following administration of the cells, the biological activity of the engineered cell populations in some embodiments is measured, e.g., by any of a number of known methods. Parameters to assess include specific binding of an engineered or natural T cell or other immune cell to antigen, in vivo, e.g., by imaging, or ex vivo, e.g., by ELISA or flow cytometry. In certain embodiments, the ability of the engineered cells to destroy target cells can be measured using any suitable known methods, such as cytotoxicity assays described in, for example, Kochenderfer et al., J. Immunotherapy, 32(7): 689-702 (2009), and Herman et al. J. Immunological Methods, 285(1): 25-40 (2004). In certain embodiments, the biological activity of the cells is measured by assaying expression and/or secretion of one or more cytokines, such as CD107a, IFNγ, IL-2, and TNF. In some aspects the biological activity is measured by assessing clinical outcome, such as reduction in tumor burden or load.

In certain embodiments, the engineered cells are further modified in any number of ways, such that their therapeutic or prophylactic efficacy is increased. For example, the engineered CAR expressed by the population can be conjugated either directly or indirectly through a linker to a targeting moiety. The practice of conjugating compounds, e.g., the CAR, to targeting moieties is known. See, e.g., Wadwa et al., J. Drug Targeting 3: 1 1 1 (1995), and U.S. Pat. No. 5,087,616.

In some embodiments, the cells are administered as part of a combination treatment, such as simultaneously with or sequentially with, in any order, another therapeutic intervention, such as an antibody or engineered cell or receptor or agent, such as a cytotoxic or therapeutic agent. The cells in some embodiments are co-administered with one or more additional therapeutic agents or in connection with another therapeutic intervention, either simultaneously or sequentially in any order. In some contexts, the cells are co-administered with another therapy sufficiently close in time such that the cell populations enhance the effect of one or more additional therapeutic agents, or vice versa. In some embodiments, the cells are administered prior to the one or more additional therapeutic agents. In some embodiments, the cells are administered after the one or more additional therapeutic agents. In some embodiments, the one or more additional agent includes a cytokine, such as IL-2, for example, to enhance persistence.

In some embodiments, a dose of cells is administered to subjects in accord with the provided methods, and/or with the provided articles of manufacture or compositions. In some embodiments, the size or timing of the doses is determined as a function of the particular disease or condition in the subject. In some cases, the size or timing of the doses for a particular disease in view of the provided description may be empirically determined.

In some embodiments, the dose of cells comprises between at or about 2×10⁵ of the cells/kg and at or about 2×10⁶ of the cells/kg, such as between at or about 4×10⁵ of the cells/kg and at or about 1×10⁶ of the cells/kg or between at or about 6×10⁵ of the cells/kg and at or about 8×10⁵ of the cells/kg. In some embodiments, the dose of cells comprises no more than 2×10⁵ of the cells (e.g. antigen-expressing, such as CAR-expressing cells) per kilogram body weight of the subject (cells/kg), such as no more than at or about 3×10⁵ cells/kg, no more than at or about 4×10⁵ cells/kg, no more than at or about 5×10⁵ cells/kg, no more than at or about 6×10⁵ cells/kg, no more than at or about 7×10⁵ cells/kg, no more than at or about 8×10⁵ cells/kg, no more than at or about 9×10⁵ cells/kg, no more than at or about 1×10⁶ cells/kg, or no more than at or about 2×10⁶ cells/kg. In some embodiments, the dose of cells comprises at least or at least about or at or about 2×10⁵ of the cells (e.g. antigen-expressing, such as CAR-expressing cells) per kilogram body weight of the subject (cells/kg), such as at least or at least about or at or about 3×10⁵ cells/kg, at least or at least about or at or about 4×10⁵ cells/kg, at least or at least about or at or about 5×10⁵ cells/kg, at least or at least about or at or about 6×10⁵ cells/kg, at least or at least about or at or about 7×10⁵ cells/kg, at least or at least about or at or about 8×10⁵ cells/kg, at least or at least about or at or about 9×10⁵ cells/kg, at least or at least about or at or about 1×10⁶ cells/kg, or at least or at least about or at or about 2×10⁶ cells/kg.

In certain embodiments, the cells, or individual populations of sub-types of cells, are administered to the subject at a range of at or about 0.1 million to at or about 100 billion cells and/or that amount of cells per kilogram of body weight of the subject, such as, e.g., at or about 0.1 million to at or about 50 billion cells (e.g., at or about 5 million cells, at or about 25 million cells, at or about 500 million cells, at or about 1 billion cells, at or about 5 billion cells, at or about 20 billion cells, at or about 30 billion cells, at or about 40 billion cells, or a range defined by any two of the foregoing values), at or about 1 million to at or about 50 billion cells (e.g., at or about 5 million cells, at or about 25 million cells, at or about 500 million cells, at or about 1 billion cells, at or about 5 billion cells, at or about 20 billion cells, at or about 30 billion cells, at or about 40 billion cells, or a range defined by any two of the foregoing values), such as at or about 10 million to at or about 100 billion cells (e.g., at or about 20 million cells, at or about 30 million cells, at or about 40 million cells, at or about 60 million cells, at or about 70 million cells, at or about 80 million cells, at or about 90 million cells, at or about 10 billion cells, at or about 25 billion cells, at or about 50 billion cells, at or about 75 billion cells, at or about 90 billion cells, or a range defined by any two of the foregoing values), and in some cases at or about 100 million cells to at or about 50 billion cells (e.g., at or about 120 million cells, at or about 250 million cells, at or about 350 million cells, at or about 650 million cells, at or about 800 million cells, at or about 900 million cells, at or about 3 billion cells, at or about 30 billion cells, at or about 45 billion cells) or any value in between these ranges and/or per kilogram of body weight of the subject. Dosages may vary depending on attributes particular to the disease or disorder and/or patient and/or other treatments. In some embodiments, such values refer to numbers of recombinant receptor-expressing cells; in other embodiments, they refer to number of T cells or PBMCs or total cells administered.

In some embodiments, for example, where the subject is a human, the dose includes fewer than about 5×10⁸ total recombinant receptor (e.g., CAR)-expressing cells, T cells, or peripheral blood mononuclear cells (PBMCs), e.g., in the range of at or about 1×10⁶ to at or about 5×10⁸ such cells, such as at or about 2×10⁶, 5×10⁶, 1×10⁷, 5×10⁷, 1×10⁸, 1.5×10⁸, or 5×10⁸ total such cells, or the range between any two of the foregoing values. In some embodiments, for example, where the subject is a human, the dose includes more than at or about 1×10⁶ total recombinant receptor (e.g., CAR)-expressing cells, T cells, or peripheral blood mononuclear cells (PBMCs) and fewer than at or about 2×10⁹ total recombinant receptor (e.g., CAR)-expressing cells, T cells, or peripheral blood mononuclear cells (PBMCs), e.g., in the range of at or about 2.5×10⁷ to at or about 1.2×10⁸ such cells, such as at or about 2.5×10⁷, 5×10⁷, 1×10⁸, 1.5×10⁸, 8×10⁸, or 1.2×10⁸ total such cells, or the range between any two of the foregoing values.

In some embodiments, the dose of genetically engineered cells comprises from at or about 1×10⁵ to at or about 5×10⁸ total CAR-expressing (CAR⁺) T cells, from at or about 1×10⁵ to at or about 2.5×10⁸ total CAR⁺ T cells, from at or about 1×10⁵ to at or about 1×10⁸ total CAR⁺ T cells, from at or about 1×10⁵ to at or about 5×10⁷ total CAR⁺ T cells, from at or about 1×10⁵ to at or about 2.5×10⁷ total CAR⁺ T cells, from at or about 1×10⁵ to at or about 1×10⁷ total CAR⁺ T cells, from at or about 1×10⁵ to at or about 5×10⁶ total CAR⁺ T cells, from at or about 1×10⁵ to at or about 2.5×10⁶ total CAR⁺ T cells, from at or about 1×10⁵ to at or about 1×10⁶ total CAR⁺ T cells, from at or about 1×10⁶ to at or about 5×10⁸ total CAR⁺ T cells, from at or about 1×10⁶ to at or about 2.5×10⁸ total CAR⁺ T cells, from at or about 1×10⁶ to at or about 1×10⁸ total CAR⁺ T cells, from at or about 1×10⁶ to at or about 5×10⁷ total CAR⁺ T cells, from at or about 1×10⁶ to at or about 2.5×10⁷ total CAR⁺ T cells, from at or about 1×10⁶ to at or about 1×10⁷ total CAR⁺ T cells, from at or about 1×10⁶ to at or about 5×10⁶ total CAR⁺ T cells, from at or about 1×10⁶ to at or about 2.5×10⁶ total CAR⁺ T cells, from at or about 2.5×10⁶ to at or about 5×10⁸ total CAR⁺ T cells, from at or about 2.5×10⁶ to at or about 2.5×10⁸ total CAR⁺ T cells, from at or about 2.5×10⁶ to at or about 1×10⁸ total CAR⁺ T cells, from at or about 2.5×10⁶ to at or about 5×10⁷ total CAR⁺ T cells, from at or about 2.5×10⁶ to at or about 2.5×10⁷ total CAR⁺ T cells, from at or about 2.5×10⁶ to at or about 1×10⁷ total CAR⁺ T cells, from at or about 2.5×10⁶ to at or about 5×10⁶ total CAR⁺ T cells, from at or about 5×10⁶ to at or about 5×10⁸ total CAR⁺ T cells, from at or about 5×10⁶ to at or about 2.5×10⁸ total CAR⁺ T cells, from at or about 5×10⁶ to at or about 1×10⁸ total CAR⁺ T cells, from at or about 5×10⁶ to at or about 5×10⁷ total CAR⁺ T cells, from at or about 5×10⁶ to at or about 2.5×10⁷ total CAR⁺ T cells, from at or about 5×10⁶ to at or about 1×10⁷ total CAR⁺ T cells, from at or about 1×10⁷ to at or about 5×10⁸ total CAR⁺ T cells, from at or about 1×10⁷ to at or about 2.5×10⁸ total CAR⁺ T cells, from at or about 1×10⁷ to at or about 1×10⁸ total CAR⁺ T cells, from at or about 1×10⁷ to at or about 5×10⁷ total CAR⁺ T cells, from at or about 1×10⁷ to at or about 2.5×10⁷ total CAR⁺ T cells, from at or about 2.5×10⁷ to at or about 5×10⁸ total CAR⁺ T cells, from at or about 2.5×10⁷ to at or about 2.5×10⁸ total CAR⁺ T cells, from at or about 2.5×10⁷ to at or about 1×10⁸ total CAR⁺ T cells, from at or about 2.5×10⁷ to at or about 5×10⁷ total CAR⁺ T cells, from at or about 5×10⁷ to at or about 5×10⁸ total CAR⁺ T cells, from at or about 5×10⁷ to at or about 2.5×10⁸ total CAR⁺ T cells, from at or about 5×10⁷ to at or about 1×10⁸ total CAR⁺ T cells, from at or about 1×10⁸ to at or about 5×10⁸ total CAR⁺ T cells, from at or about 1×10⁸ to at or about 2.5×10⁸ total CAR⁺ T cells, from at or about or 2.5×10⁸ to at or about 5×10⁸ total CAR⁺ T cells. In some embodiments, the dose of genetically engineered cells comprises from or from about 2.5×10⁷ to at or about 1.5×10⁸ total CAR⁺ T cells, such as from or from about 5×10⁷ to or to about 1×10⁸ total CAR⁺ T cells.

In some embodiments, the dose of genetically engineered cells comprises at least at or about 1×10⁵ CAR⁺ cells, at least at or about 2.5×10⁵ CAR⁺ cells, at least at or about 5×10⁵ CAR⁺ cells, at least at or about 1×10⁶ CAR⁺ cells, at least at or about 2.5×10⁶ CAR⁺ cells, at least at or about 5×10⁶ CAR⁺ cells, at least at or about 1×10⁷ CAR⁺ cells, at least at or about 2.5×10⁷ CAR⁺ cells, at least at or about 5×10⁷ CAR⁺ cells, at least at or about 1×10⁸ CAR⁺ cells, at least at or about 1.5×10⁸ CAR⁺ cells, at least at or about 2.5×10⁸ CAR⁺ cells, or at least at or about 5×10⁸ CAR⁺ cells.

In some embodiments, the cell therapy comprises administration of a dose comprising a number of cell from or from about 1×10⁵ to or to about 5×10⁸ total recombinant receptor-expressing cells, total T cells, or total peripheral blood mononuclear cells (PBMCs), from or from about 5×10⁵ to or to about 1×10⁷ total recombinant receptor-expressing cells, total T cells, or total peripheral blood mononuclear cells (PBMCs) or from or from about 1×10⁶ to or to about 1×10⁷ total recombinant receptor-expressing cells, total T cells, or total peripheral blood mononuclear cells (PBMCs), each inclusive. In some embodiments, the cell therapy comprises administration of a dose of cells comprising a number of cells at least or at least about 1×10⁵ total recombinant receptor-expressing cells, total T cells, or total peripheral blood mononuclear cells (PBMCs), such at least or at least 1×10⁶, at least or at least about 1×10⁷, at least or at least about 1×10⁸ of such cells. In some embodiments, the number is with reference to the total number of CD3⁺ or CD8⁺, in some cases also recombinant receptor-expressing (e.g. CAR⁺) cells. In some embodiments, the cell therapy comprises administration of a dose comprising a number of cell from or from about 1×10⁵ to or to about 5×10⁸ CD3⁺ or CD8⁺ total T cells or CD3⁺ or CD8⁺ recombinant receptor-expressing cells, from or from about 5×10⁵ to or to about 1×10⁷ CD3⁺ or CD8⁺ total T cells or CD3⁺ or CD8⁺ recombinant receptor-expressing cells, or from or from about 1×10⁶ to or to about 1×10⁷ CD3⁺ or CD8⁺ total T cells or CD3⁺ or CD8⁺ recombinant receptor-expressing cells, each inclusive. In some embodiments, the cell therapy comprises administration of a dose comprising a number of cell from or from about 1×10⁵ to or to about 5×10⁸ total CD3⁺/CAR⁺ or CD8⁺/CAR⁺ cells, from or from about 5×10⁵ to or to about 1×10⁷ total CD3⁺/CAR⁺ or CD8⁺/CAR⁺ cells, or from or from about 1×10⁶ to or to about 1×10⁷ total CD3⁺/CAR⁺ or CD8⁺/CAR⁺ cells, each inclusive.

In some embodiments, the T cells of the dose include CD4⁺ T cells, CD8⁺ T cells or CD4⁺ and CD8⁺ T cells.

In some embodiments, for example, where the subject is human, the CD8⁺ T cells of the dose, including in a dose including CD4⁺ and CD8⁺ T cells, includes between at or about 1×10⁶ and at or about 5×10⁸ total recombinant receptor (e.g., CAR)-expressing CD8⁺ cells, e.g., in the range of from at or about 5×10⁶ to at or about 1×10⁸ such cells, such as 1×10⁷, 2.5×10⁷, 5×10⁷, 7.5×10⁷, 1×10⁸, 1.5×10⁸, or 5×10⁸ total such cells, or the range between any two of the foregoing values. In some embodiments, the patient is administered multiple doses, and each of the doses or the total dose can be within any of the foregoing values. In some embodiments, the dose of cells comprises the administration of from or from about 1×10⁷ to or to about 0.75×10⁸ total recombinant receptor-expressing CD8⁺ T cells, from or from about 1×10⁷ to or to about 5×10⁷ total recombinant receptor-expressing CD8⁺ T cells, from or from about 1×10⁷ to or to about 0.25×10⁸ total recombinant receptor-expressing CD8⁺ T cells, each inclusive. In some embodiments, the dose of cells comprises the administration of at or about 1×10⁷, 2.5×10⁷, 5×10⁷, 7.5×10⁷, 1×10⁸, 1.5×10⁸, 2.5×10⁸, or 5×10⁸ total recombinant receptor-expressing CD8⁺ T cells.

In some embodiments, the dose of cells, e.g., recombinant receptor-expressing T cells, is administered to the subject as a single dose or is administered only one time within a period of two weeks, one month, three months, six months, 1 year or more.

In the context of adoptive cell therapy, administration of a given “dose” encompasses administration of the given amount or number of cells as a single composition and/or single uninterrupted administration, e.g., as a single injection or continuous infusion, and also encompasses administration of the given amount or number of cells as a split dose or as a plurality of compositions, provided in multiple individual compositions or infusions, over a specified period of time, such as over no more than 3 days. Thus, in some contexts, the dose is a single or continuous administration of the specified number of cells, given or initiated at a single point in time. In some contexts, however, the dose is administered in multiple injections or infusions over a period of no more than three days, such as once a day for three days or for two days or by multiple infusions over a single day period.

Thus, in some aspects, the cells of the dose are administered in a single pharmaceutical composition. In some embodiments, the cells of the dose are administered in a plurality of compositions, collectively containing the cells of the dose.

In some embodiments, the term “split dose” refers to a dose that is split so that it is administered over more than one day. This type of dosing is encompassed by the present methods and is considered to be a single dose.

Thus, the dose of cells may be administered as a split dose, e.g., a split dose administered over time. For example, in some embodiments, the dose may be administered to the subject over 2 days or over 3 days. Exemplary methods for split dosing include administering 25% of the dose on the first day and administering the remaining 75% of the dose on the second day. In other embodiments, 33% of the dose may be administered on the first day and the remaining 67% administered on the second day. In some aspects, 10% of the dose is administered on the first day, 30% of the dose is administered on the second day, and 60% of the dose is administered on the third day. In some embodiments, the split dose is not spread over more than 3 days.

In some embodiments, cells of the dose may be administered by administration of a plurality of compositions or solutions, such as a first and a second, optionally more, each containing some cells of the dose. In some aspects, the plurality of compositions, each containing a different population and/or sub-types of cells, are administered separately or independently, optionally within a certain period of time. For example, the populations or sub-types of cells can include CD8⁺ and CD4⁺ T cells, respectively, and/or CD8+- and CD4+-enriched populations, respectively, e.g., CD4+ and/or CD8+ T cells each individually including cells genetically engineered to express the recombinant receptor. In some embodiments, the administration of the dose comprises administration of a first composition comprising a dose of CD8+ T cells or a dose of CD4+ T cells and administration of a second composition comprising the other of the dose of CD4+ T cells and the CD8+ T cells.

In some embodiments, the administration of the composition or dose, e.g., administration of the plurality of cell compositions, involves administration of the cell compositions separately. In some aspects, the separate administrations are carried out simultaneously, or sequentially, in any order. In some embodiments, the dose comprises a first composition and a second composition, and the first composition and second composition are administered from at or about 0 to at or about 12 hours apart, from at or about 0 to at or about 6 hours apart or from at or about 0 to at or about 2 hours apart. In some embodiments, the initiation of administration of the first composition and the initiation of administration of the second composition are carried out no more than at or about 2 hours, no more than at or about 1 hour, or no more than at or about 30 minutes apart, no more than at or about 15 minutes, no more than at or about 10 minutes or no more than at or about 5 minutes apart. In some embodiments, the initiation and/or completion of administration of the first composition and the completion and/or initiation of administration of the second composition are carried out no more than at or about 2 hours, no more than at or about 1 hour, or no more than at or about 30 minutes apart, no more than at or about 15 minutes, no more than at or about 10 minutes or no more than at or about 5 minutes apart.

In some composition, the first composition, e.g., first composition of the dose, comprises CD4+ T cells. In some composition, the first composition, e.g., first composition of the dose, comprises CD8+ T cells. In some embodiments, the first composition is administered prior to the second composition.

In some embodiments, the dose or composition of cells includes a defined or target ratio of CD4+ cells expressing a recombinant receptor to CD8+ cells expressing a recombinant receptor and/or of CD4+ cells to CD8+ cells, which ratio optionally is approximately 1:1 or is between approximately 1:3 and approximately 3:1, such as approximately 1:1. In some aspects, the administration of a composition or dose with the target or desired ratio of different cell populations (such as CD4+:CD8+ ratio or CAR+CD4+:CAR+CD8+ ratio, e.g., 1:1) involves the administration of a cell composition containing one of the populations and then administration of a separate cell composition comprising the other of the populations, where the administration is at or approximately at the target or desired ratio. In some aspects, administration of a dose or composition of cells at a defined ratio leads to improved expansion, persistence and/or antitumor activity of the T cell therapy.

In some embodiments, the subject receives multiple doses, e.g., two or more doses or multiple consecutive doses, of the cells. In some embodiments, two doses are administered to a subject. In some embodiments, the subject receives the consecutive dose, e.g., second dose, is administered approximately 4, 5, 6, 7, 8, 9, 10, 11, 12, 13, 14, 15, 16, 17, 18, 19, 20 or 21 days after the first dose. In some embodiments, multiple consecutive doses are administered following the first dose, such that an additional dose or doses are administered following administration of the consecutive dose. In some aspects, the number of cells administered to the subject in the additional dose is the same as or similar to the first dose and/or consecutive dose. In some embodiments, the additional dose or doses are larger than prior doses.

In some aspects, the size of the first and/or consecutive dose is determined based on one or more criteria such as response of the subject to prior treatment, e.g. chemotherapy, disease burden in the subject, such as tumor load, bulk, size, or degree, extent, or type of metastasis, stage, and/or likelihood or incidence of the subject developing toxic outcomes, e.g., CRS, macrophage activation syndrome, tumor lysis syndrome, neurotoxicity, and/or a host immune response against the cells and/or recombinant receptors being administered.

In some aspects, the time between the administration of the first dose and the administration of the consecutive dose is about 9 to about 35 days, about 14 to about 28 days, or 15 to 27 days. In some embodiments, the administration of the consecutive dose is at a time point more than about 14 days after and less than about 28 days after the administration of the first dose. In some aspects, the time between the first and consecutive dose is about 21 days. In some embodiments, an additional dose or doses, e.g. consecutive doses, are administered following administration of the consecutive dose. In some aspects, the additional consecutive dose or doses are administered at least about 14 and less than about 28 days following administration of a prior dose. In some embodiments, the additional dose is administered less than about 14 days following the prior dose, for example, 4, 5, 6, 7, 8, 9, 10, 11, 12, or 13 days after the prior dose. In some embodiments, no dose is administered less than about 14 days following the prior dose and/or no dose is administered more than about 28 days after the prior dose.

In some embodiments, the dose of cells, e.g., recombinant receptor-expressing cells, comprises two doses (e.g., a double dose), comprising a first dose of the T cells and a consecutive dose of the T cells, wherein one or both of the first dose and the second dose comprises administration of the split dose of T cells.

In some embodiments, the dose of cells is generally large enough to be effective in reducing disease burden.

In some embodiments, the cells are administered at a desired dosage, which in some aspects includes a desired dose or number of cells or cell type(s) and/or a desired ratio of cell types. Thus, the dosage of cells in some embodiments is based on a total number of cells (or number per kg body weight) and a desired ratio of the individual populations or sub-types, such as the CD4+ to CD8+ ratio. In some embodiments, the dosage of cells is based on a desired total number (or number per kg of body weight) of cells in the individual populations or of individual cell types. In some embodiments, the dosage is based on a combination of such features, such as a desired number of total cells, desired ratio, and desired total number of cells in the individual populations.

In some embodiments, the populations or sub-types of cells, such as CD8⁺ and CD4⁺ T cells, are administered at or within a tolerated difference of a desired dose of total cells, such as a desired dose of T cells. In some aspects, the desired dose is a desired number of cells or a desired number of cells per unit of body weight of the subject to whom the cells are administered, e.g., cells/kg. In some aspects, the desired dose is at or above a minimum number of cells or minimum number of cells per unit of body weight. In some aspects, among the total cells, administered at the desired dose, the individual populations or sub-types are present at or near a desired output ratio (such as CD4⁺ to CD8⁺ ratio), e.g., within a certain tolerated difference or error of such a ratio.

In some embodiments, the cells are administered at or within a tolerated difference of a desired dose of one or more of the individual populations or sub-types of cells, such as a desired dose of CD4+ cells and/or a desired dose of CD8+ cells. In some aspects, the desired dose is a desired number of cells of the sub-type or population, or a desired number of such cells per unit of body weight of the subject to whom the cells are administered, e.g., cells/kg. In some aspects, the desired dose is at or above a minimum number of cells of the population or sub-type, or minimum number of cells of the population or sub-type per unit of body weight.

Thus, in some embodiments, the dosage is based on a desired fixed dose of total cells and a desired ratio, and/or based on a desired fixed dose of one or more, e.g., each, of the individual sub-types or sub-populations. Thus, in some embodiments, the dosage is based on a desired fixed or minimum dose of T cells and a desired ratio of CD4⁺ to CD8⁺ cells, and/or is based on a desired fixed or minimum dose of CD4⁺ and/or CD8⁺ cells.

In some embodiments, the cells are administered at or within a tolerated range of a desired output ratio of multiple cell populations or sub-types, such as CD4+ and CD8+ cells or sub-types. In some aspects, the desired ratio can be a specific ratio or can be a range of ratios. for example, in some embodiments, the desired ratio (e.g., ratio of CD4⁺ to CD8⁺ cells) is between at or about 5:1 and at or about 5:1 (or greater than about 1:5 and less than about 5:1), or between at or about 1:3 and at or about 3:1 (or greater than about 1:3 and less than about 3:1), such as between at or about 2:1 and at or about 1:5 (or greater than about 1:5 and less than about 2:1, such as at or about 5:1, 4.5:1, 4:1, 3.5:1, 3:1, 2.5:1, 2:1, 1.9:1, 1.8:1, 1.7:1, 1.6:1, 1.5:1, 1.4:1, 1.3:1, 1.2:1, 1.1:1, 1:1, 1:1.1, 1:1.2, 1:1.3, 1:1.4, 1:1.5, 1:1.6, 1:1.7, 1:1.8, 1:1.9: 1:2, 1:2.5, 1:3, 1:3.5, 1:4, 1:4.5, or 1:5. In some aspects, the tolerated difference is within about 1%, about 2%, about 3%, about 4% about 5%, about 10%, about 15%, about 20%, about 25%, about 30%, about 35%, about 40%, about 45%, about 50% of the desired ratio, including any value in between these ranges.

In particular embodiments, the numbers and/or concentrations of cells refer to the number of recombinant receptor (e.g., CAR)-expressing cells. In other embodiments, the numbers and/or concentrations of cells refer to the number or concentration of all cells, T cells, or peripheral blood mononuclear cells (PBMCs) administered.

In some aspects, the size of the dose is determined based on one or more criteria such as response of the subject to prior treatment, e.g. chemotherapy, disease burden in the subject, such as tumor load, bulk, size, or degree, extent, or type of metastasis, stage, and/or likelihood or incidence of the subject developing toxic outcomes, e.g., CRS, macrophage activation syndrome, tumor lysis syndrome, neurotoxicity, and/or a host immune response against the cells and/or recombinant receptors being administered.

In some embodiments, the methods also include administering one or more additional doses of cells expressing a chimeric antigen receptor (CAR) and/or lymphodepleting therapy, and/or one or more steps of the methods are repeated. In some embodiments, the one or more additional dose is the same as the initial dose. In some embodiments, the one or more additional dose is different from the initial dose, e.g., higher, such as 2-fold, 3-fold, 4-fold, 5-fold, 6-fold, 7-fold, 8-fold, 9-fold or 10-fold or more higher than the initial dose, or lower, such as e.g., higher, such as 2-fold, 3-fold, 4-fold, 5-fold, 6-fold, 7-fold, 8-fold, 9-fold or 10-fold or more lower than the initial dose. In some embodiments, administration of one or more additional doses is determined based on response of the subject to the initial treatment or any prior treatment, disease burden in the subject, such as tumor load, bulk, size, or degree, extent, or type of metastasis, stage, and/or likelihood or incidence of the subject developing toxic outcomes, e.g., CRS, macrophage activation syndrome, tumor lysis syndrome, neurotoxicity, and/or a host immune response against the cells being administered.

### V. PHARMACEUTICAL COMPOSITION AND FORMULATION

Also provided are compositions, such as pharmaceutical compositions and formulations for administration, such as for adoptive cell therapy. In some aspects, the pharmaceutical compositions contain any of the engineered cells or compositions containing the engineered cells described herein, e.g., comprising a modified TGFBR2 locus comprising a transgene sequence encoding a recombinant or chimeric receptor. In some embodiments, the dose of cells comprising the provided engineered cells, e.g., comprising a modified TGFBR2 locus comprising a transgene sequence encoding a recombinant antigen receptor or a portion thereof, e.g. CAR, is provided as a composition or formulation, such as a pharmaceutical composition or formulation. Such compositions can be used in accord with the provided methods, and/or with the provided articles of manufacture or compositions, such as in the prevention or treatment of diseases, conditions, and disorders, or in detection, diagnostic, and prognostic methods.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

In some aspects, the choice of carrier is determined in part by the particular cell or agent and/or by the method of administration. Accordingly, there are a variety of suitable formulations. For example, the pharmaceutical composition can contain preservatives. Suitable preservatives may include, for example, methylparaben, propylparaben, sodium benzoate, and benzalkonium chloride. In some aspects, a mixture of two or more preservatives is used. The preservative or mixtures thereof are typically present in an amount of about 0.0001% to about 2% by weight of the total composition. Carriers are described, e.g., by Remington's Pharmaceutical Sciences 16th edition, Osol, A. Ed. (1980). Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG).

Buffering agents in some aspects are included in the compositions. Suitable buffering agents include, for example, citric acid, sodium citrate, phosphoric acid, potassium phosphate, and various other acids and salts. In some aspects, a mixture of two or more buffering agents is used. The buffering agent or mixtures thereof are typically present in an amount of about 0.001% to about 4% by weight of the total composition. Methods for preparing administrable pharmaceutical compositions are known. Exemplary methods are described in more detail in, for example, Remington: The Science and Practice of Pharmacy, Lippincott Williams & Wilkins; 21st ed. (May 1, 2005).

The formulation or composition may also contain more than one active ingredient useful for the particular indication, disease, or condition being prevented or treated with the cells or agents, where the respective activities do not adversely affect one another. Such active ingredients are suitably present in combination in amounts that are effective for the purpose intended. Thus, in some embodiments, the pharmaceutical composition further includes other pharmaceutically active agents or drugs, such as chemotherapeutic agents, e.g., asparaginase, busulfan, carboplatin, cisplatin, daunorubicin, doxorubicin, fluorouracil, gemcitabine, hydroxyurea, methotrexate, paclitaxel, rituximab, vinblastine, vincristine, etc. In some embodiments, the agents or cells are administered in the form of a salt, e.g., a pharmaceutically acceptable salt. Suitable pharmaceutically acceptable acid addition salts include those derived from mineral acids, such as hydrochloric, hydrobromic, phosphoric, metaphosphoric, nitric, and sulphuric acids, and organic acids, such as tartaric, acetic, citric, malic, lactic, fumaric, benzoic, glycolic, gluconic, succinic, and arylsulphonic acids, for example, p-toluenesulphonic acid.

The pharmaceutical composition in some embodiments contains agents or cells in amounts effective to treat or prevent the disease or condition, such as a therapeutically effective or prophylactically effective amount. Therapeutic or prophylactic efficacy in some embodiments is monitored by periodic assessment of treated subjects. For repeated administrations over several days or longer, depending on the condition, the treatment is repeated until a desired suppression of disease symptoms occurs. However, other dosage regimens may be useful and can be determined. The desired dosage can be delivered by a single bolus administration of the composition, by multiple bolus administrations of the composition, or by continuous infusion administration of the composition.

The agents or cells can be administered by any suitable means, for example, by bolus infusion, by injection, e.g., intravenous or subcutaneous injections, intraocular injection, periocular injection, subretinal injection, intravitreal injection, trans-septal injection, subscleral injection, intrachoroidal injection, intracameral injection, subconjectval injection, subconjuntival injection, sub-Tenon's injection, retrobulbar injection, peribulbar injection, or posterior juxtascleral delivery. In some embodiments, they are administered by parenteral, intrapulmonary, and intranasal, and, if desired for local treatment, intralesional administration. Parenteral infusions include intramuscular, intravenous, intraarterial, intraperitoneal, or subcutaneous administration. In some embodiments, a given dose is administered by a single bolus administration of the cells or agent. In some embodiments, it is administered by multiple bolus administrations of the cells or agent, for example, over a period of no more than 3 days, or by continuous infusion administration of the cells or agent.

For the prevention or treatment of disease, the appropriate dosage may depend on the type of disease to be treated, the type of agent or agents, the type of cells or recombinant receptors, the severity and course of the disease, whether the agent or cells are administered for preventive or therapeutic purposes, previous therapy, the subject's clinical history and response to the agent or the cells, and the discretion of the attending physician. The compositions are in some embodiments suitably administered to the subject at one time or over a series of treatments.

The cells or agents may be administered using standard administration techniques, formulations, and/or devices. Provided are formulations and devices, such as syringes and vials, for storage and administration of the compositions. With respect to cells, administration can be autologous or heterologous. In some aspects, the cells are isolated from a subject, engineered, and administered to the same subject. In other aspects, they are isolated from one subject, engineered, and administered to another subject. For example, immunoresponsive cells or progenitors can be obtained from one subject, and administered to the same subject or a different, compatible subject. Peripheral blood derived immunoresponsive cells or their progeny (e.g., in vivo, ex vivo or in vitro derived) can be administered via localized injection, including catheter administration, systemic injection, localized injection, intravenous injection, or parenteral administration. When administering a therapeutic composition (e.g., a pharmaceutical composition containing a genetically modified immunoresponsive cell or an agent that treats or ameliorates symptoms of neurotoxicity), it will generally be formulated in a unit dosage injectable form (solution, suspension, emulsion).

Formulations include those for oral, intravenous, intraperitoneal, subcutaneous, pulmonary, transdermal, intramuscular, intranasal, buccal, sublingual, or suppository administration. In some embodiments, the agent or cell populations are administered parenterally. The term “parenteral,” as used herein, includes intravenous, intramuscular, subcutaneous, rectal, vaginal, and intraperitoneal administration. In some embodiments, the agent or cell populations are administered to a subject using peripheral systemic delivery by intravenous, intraperitoneal, or subcutaneous injection.

Compositions in some embodiments are provided as sterile liquid preparations, e.g., isotonic aqueous solutions, suspensions, emulsions, dispersions, or viscous compositions, which may in some aspects be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol) and suitable mixtures thereof.

Sterile injectable solutions can be prepared by incorporating the agent or cells in a solvent, such as in admixture with a suitable carrier, diluent, or excipient such as sterile water, physiological saline, glucose, dextrose, or the like.

The formulations to be used for in vivo administration are generally sterile. Sterility may be readily accomplished, e.g., by filtration through sterile filtration membranes.

### VI. KITS AND ARTICLES OF MANUFACTURE

Also provided are articles of manufacture, systems, apparatuses, and kits useful in performing the provided embodiments. In some embodiments, the provided articles of manufacture or kits contain one or more components of the one or more agent(s) capable of inducing genetic disruption and/or template polynucleotide(s), e.g., template polynucleotides containing transgene sequences encoding a recombinant receptor or a portion thereof. In some embodiments, the articles of manufacture or kits can be used in methods for engineering T cells to express a recombinant receptor and/or other molecules as described herein, for example, to generate the engineered cells comprising a modified TGFBR2 locus comprising a transgene encoding a recombinant receptor or a portion thereof.

In some embodiments, the articles of manufacture or kits include polypeptides, nucleic acids, vectors and/or polynucleotides useful in performing the provided methods. In some embodiments, the articles of manufacture or kits include one or more agent(s) capable of inducing a genetic disruption, for example, at a TGFBR2 locus (such as those described in Section I.A herein). In some embodiments, the articles of manufacture or kits include one or more nucleic acid molecules, e.g., a plasmid or a DNA fragment, that encodes one or more components of the one or more agent(s) capable of inducing genetic disruption and/or comprises template polynucleotide(s), e.g., for use in targeting transgene sequences into the cell via HDR, such as those described in Section I.B.2 herein. In some embodiments, the articles of manufacture or kits provided herein contain control vectors.

In some embodiments, the articles of manufacture or kits provided herein contain one or more agent(s), wherein each of the one or more agent is independently capable of inducing a genetic disruption of a target site within a TGFBR2 locus; and a template polynucleotide comprising a transgene encoding a recombinant receptor or a portion thereof, wherein the transgene is targeted for integration at or near the target site via homology directed repair (HDR). In some aspects, the one or more agent(s) capable of inducing a genetic disruption is any described herein. In some aspects, the one or more agent(s) is a ribonucleoprotein (RNP) complex comprising a Cas9/gRNA complex. In some aspects, the gRNA included in the RNP targets a target site in the TGFBR2 locus, such as any target site described herein. In some aspects, the template polynucleotide is any of the template polynucleotide described herein.

In some embodiments, the articles of manufacture or kits include one or more containers, typically a plurality of containers, packaging material, and a label or package insert on or associated with the container or containers and/or packaging, generally including instructions for use, e.g., instructions for introducing the components into the cells for engineering.

The articles of manufacture provided herein contain packaging materials. Packaging materials for use in packaging the provided materials are well known. See, for example, U.S. Pat. Nos. 5,323,907, 5,052,558 and 5,033,252, each of which is incorporated herein in its entirety. Examples of packaging materials include, but are not limited to, blister packs, bottles, tubes, inhalers, pumps, bags, vials, containers, syringes, disposable laboratory supplies, e.g., pipette tips and/or plastic plates, or bottles. The articles of manufacture or kits can include a device so as to facilitate dispensing of the materials or to facilitate use in a high-throughput or large-scale manner, e.g., to facilitate use in robotic equipment. Typically, the packaging is non-reactive with the compositions contained therein.

In some embodiments, the one or more agent(s) capable of inducing genetic disruption and/or template polynucleotide(s) are packaged separately. In some embodiments, each container can have a single compartment. In some embodiments, other components of the articles of manufacture or kits are packaged separately, or together in a single compartment.

Also provided are articles of manufacture, systems, apparatuses, and kits useful in administering the provided cells and/or cell compositions, e.g., for use in therapy or treatment. In some embodiments, the articles of manufacture or kits provided herein contain T cells and/or T cell compositions, such as any T cells and/or T cell compositions described herein. In some aspects, the articles of manufacture or kits provided herein can be used for administration of the T cells or T cell compositions, and can include instructions for use.

In some embodiments, the articles of manufacture or kits provided herein contain T cells, and/or T cell compositions, such as any T cells, and/or T cell compositions described herein. In some embodiments, the T cells, and/or T cell compositions any of the modified T cells used the screening methods described herein. In some embodiments, the articles of manufacture or kits provided herein contain control or unmodified T cells and/or T cell compositions. In some embodiments, the article of manufacture or kits include one or more instructions for administration of the engineered cells and/or cell compositions for therapy.

The articles of manufacture and/or kits containing cells or cell compositions for therapy, may include a container and a label or package insert on or associated with the container. Suitable containers include, for example, bottles, vials, syringes, IV solution bags, etc. The containers may be formed from a variety of materials such as glass or plastic. The container in some embodiments holds a composition which is by itself or combined with another composition effective for treating, preventing and/or diagnosing the condition. In some embodiments, the container has a sterile access port. Exemplary containers include an intravenous solution bags, vials, including those with stoppers pierceable by a needle for injection, or bottles or vials for orally administered agents. The label or package insert may indicate that the composition is used for treating a disease or condition. The article of manufacture may include (a) a first container with a composition contained therein, wherein the composition includes engineered cells expressing a recombinant receptor; and (b) a second container with a composition contained therein, wherein the composition includes the second agent. In some embodiments, the article of manufacture may include (a) a first container with a first composition contained therein, wherein the composition includes a subtype of engineered cells expressing a recombinant receptor; and (b) a second container with a composition contained therein, wherein the composition includes a different subtype of engineered cells expressing a recombinant receptor. The article of manufacture may further include a package insert indicating that the compositions can be used to treat a particular condition. Alternatively, or additionally, the article of manufacture may further include another or the same container comprising a pharmaceutically-acceptable buffer. It may further include other materials such as other buffers, diluents, filters, needles, and/or syringes.

### VII. DEFINITIONS

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

As used herein, the singular forms “a,” “an,” and “the” include plural referents unless the context clearly dictates otherwise. For example, “a” or “an” means “at least one” or “one or more.” It is understood that aspects and variations described herein include “consisting” and/or “consisting essentially of” aspects and variations.

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be included in the smaller ranges, and are also encompassed within the claimed subject matter, subject to any specifically excluded limit in the stated range. Where the stated range includes one or both of the limits, ranges excluding either or both of those included limits are also included in the claimed subject matter. This applies regardless of the breadth of the range.

The term “about” as used herein refers to the usual error range for the respective value readily known. Reference to “about” a value or parameter herein includes (and describes) embodiments that are directed to that value or parameter per se. For example, description referring to “about X” includes description of “X”. In some embodiments, “about” may refer to ±25%, ±20%, ±15%, ±10%, ±5%, or ±1%.

As used herein, recitation that nucleotides or amino acid positions “correspond to” nucleotides or amino acid positions in a disclosed sequence, such as set forth in the Sequence listing, refers to nucleotides or amino acid positions identified upon alignment with the disclosed sequence to maximize identity using a standard alignment algorithm, such as the GAP algorithm. By aligning the sequences, corresponding residues can be identified, for example, using conserved and identical amino acid residues as guides. In general, to identify corresponding positions, the sequences of amino acids are aligned so that the highest order match is obtained (see, e.g.: Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991; Carrillo et al. (1988) SIAM J Applied Math 48: 1073).

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.” Among the vectors are viral vectors, such as retroviral, e.g., gammaretroviral and lentiviral vectors.

The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

As used herein, a statement that a cell or population of cells is “positive” for a particular marker refers to the detectable presence on or in the cell of a particular marker, typically a surface marker. When referring to a surface marker, the term refers to the presence of surface expression as detected by flow cytometry, for example, by staining with an antibody that specifically binds to the marker and detecting said antibody, wherein the staining is detectable by flow cytometry at a level substantially above the staining detected carrying out the same procedure with an isotype-matched control under otherwise identical conditions and/or at a level substantially similar to that for cell known to be positive for the marker, and/or at a level substantially higher than that for a cell known to be negative for the marker.

As used herein, a statement that a cell or population of cells is “negative” for a particular marker refers to the absence of substantial detectable presence on or in the cell of a particular marker, typically a surface marker. When referring to a surface marker, the term refers to the absence of surface expression as detected by flow cytometry, for example, by staining with an antibody that specifically binds to the marker and detecting said antibody, wherein the staining is not detected by flow cytometry at a level substantially above the staining detected carrying out the same procedure with an isotype-matched control under otherwise identical conditions, and/or at a level substantially lower than that for cell known to be positive for the marker, and/or at a level substantially similar as compared to that for a cell known to be negative for the marker.

As used herein, “percent (%) amino acid sequence identity” and “percent identity” when used with respect to an amino acid sequence (reference polypeptide sequence) is defined as the percentage of amino acid residues in a candidate sequence (e.g., the subject antibody or fragment) that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various known ways, in some embodiments, using publicly available computer software such as BLAST, BLAST-2, ALIGN or Megalign (DNASTAR) software. Appropriate parameters for aligning sequences can be determined, including any algorithms needed to achieve maximal alignment over the full length of the sequences being compared.

In some embodiments, “operably linked” may include the association of components, such as a DNA sequence, e.g. a heterologous nucleic acid) and a regulatory sequence(s), in such a way as to permit gene expression when the appropriate molecules (e.g. transcriptional activator proteins) are bound to the regulatory sequence. Hence, it means that the components described are in a relationship permitting them to function in their intended manner.

An amino acid substitution may include replacement of one amino acid in a polypeptide with another amino acid. The substitution may be a conservative amino acid substitution or a non-conservative amino acid substitution. Amino acid substitutions may be introduced into a binding molecule, e.g., antibody, of interest and the products screened for a desired activity, e.g., retained/improved antigen binding, decreased immunogenicity, or improved ADCC or CDC.

Amino acids generally can be grouped according to the following common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

In some embodiments, conservative substitutions can involve the exchange of a member of one of these classes for another member of the same class. In some embodiments, non-conservative amino acid substitutions can involve exchanging a member of one of these classes for another class.

As used herein, a composition refers to any mixture of two or more products, substances, or compounds, including cells. It may be a solution, a suspension, liquid, powder, a paste, aqueous, non-aqueous or any combination thereof.

As used herein, a “subject” is a mammal, such as a human or other animal, and typically is human.

### VIII. EXEMPLARY EMBODIMENTS

Among the provided embodiments are:

1. A genetically engineered T cell, comprising a modified transforming growth factor-beta receptor type-2 (TGFBR2) locus, said modified TGFBR2 locus comprising a transgene sequence encoding a recombinant receptor or a portion thereof.

2. The genetically engineered T cell of embodiment 1, wherein the transgene sequence has been integrated at the endogenous TGFBR2 locus, optionally via homology directed repair (HDR).

3. The genetically engineered T cell of embodiment 1 or embodiment 2, wherein the modified TGFBR2 locus does not encode a functional TGFBRII polypeptide.

4. The genetically engineered T cell of any of embodiments 1-3, wherein the modified TGFBR2 locus does not encode a TGFBRII polypeptide or the expression of TGFBRII polypeptide is eliminated.

5. The genetically engineered T cell of any of embodiments 1-3, wherein the modified TGFBR2 locus does not encode a full length TGFBRII polypeptide or encodes a partial TGFBRII polypeptide.

6. The genetically engineered T cell of any of embodiments 1-3 and 5, wherein the modified TGFBR2 locus encodes a dominant negative TGFBRII polypeptide.

7. The genetically engineered T cell of any of embodiments 1-3, 5 and 6, wherein the encoded TGFBRII polypeptide comprises an amino acid sequence corresponding to residues 22-191 of SEQ ID NO:59 or residues 22-216 of SEQ ID NO:60, or a sequence that exhibits at least 85%, 86%, 87%, 88%, 89%, 90%, 91%, 92%, 93%, 94%, 95%, 96%, 97%, 98%, 99% or more sequence identity to an amino acid sequence corresponding to residues 22-191 of SEQ ID NO:59 or residues 22-216 of SEQ ID NO:60, or a fragment thereof.

8. The genetically engineered T cell of any of embodiments 1-3 and 5-7, wherein the transgene sequence is in-frame with one or more exons of an open reading frame or partial sequence thereof of the endogenous TGFBR2 locus.

9. The genetically engineered T cell of any of embodiments 1-8, wherein the transgene sequence is downstream of exon 1 and upstream of exon 6 of the open reading frame of the endogenous TGFBR2 locus.

10. The genetically engineered T cell of any of embodiments 1-9, wherein the transgene sequence is downstream of exon 4 and upstream of exon 6 of the open reading frame of the endogenous TGFBR2 locus.

11. The genetically engineered T cell of any of embodiments 1-10, wherein the recombinant receptor is or comprises recombinant T cell receptor (TCR).

12. The genetically engineered T cell of any of embodiments 1-11, wherein the recombinant receptor is a recombinant TCR and the transgene sequence encodes a TCR alpha (TCRα) chain, a TCR beta (TCRβ) chain or both.

13. The genetically engineered T cell of any of embodiments 1-10, wherein the recombinant receptor is or comprises a functional non-T cell receptor (non-TCR) antigen receptor.

14. The genetically engineered T cell of any of embodiments 1-10 and 13, wherein the recombinant receptor is a chimeric antigen receptor (CAR).

15. The genetically engineered T cell of embodiment 14, wherein the CAR comprises an extracellular region, a transmembrane domain, and an intracellular region.

16. The genetically engineered T cell of embodiment 15, wherein the extracellular region comprises a binding domain.

17. The genetically engineered T cell of embodiment 16, wherein the binding domain is or comprises an antibody or an antigen-binding fragment thereof.

18. The genetically engineered T cell of embodiment 16 and embodiment 17, wherein the binding domain is capable of binding to a target antigen that is associated with, specific to, or expressed on a cell or tissue of a disease, disorder or condition.

19. The genetically engineered T cell of embodiment 18, wherein the target antigen is a tumor antigen.

20. The genetically engineered T cell of embodiment 18 or embodiment 19, wherein the target antigen is selected from among αvβ6 integrin (avb6 integrin), B cell maturation antigen (BCMA), B7-H3, B7-H6, carbonic anhydrase 9 (CA9, also known as CAIX or G250), a cancer-testis antigen, cancer/testis antigen 1B (CTAG, also known as NY-ESO-1 and LAGE-2), carcinoembryonic antigen (CEA), a cyclin, cyclin A2, C—C Motif Chemokine Ligand 1 (CCL-1), CD19, CD20, CD22, CD23, CD24, CD30, CD33, CD38, CD44, CD44v6, CD44v7/8, CD123, CD133, CD138, CD171, chondroitin sulfate proteoglycan 4 (CSPG4), epidermal growth factor protein (EGFR), type III epidermal growth factor receptor mutation (EGFR vIII), epithelial glycoprotein 2 (EPG-2), epithelial glycoprotein 40 (EPG-40), ephrinB2, ephrin receptor A2 (EPHa2), estrogen receptor, Fc receptor like 5 (FCRL5; also known as Fc receptor homolog 5 or FCRH5), fetal acetylcholine receptor (fetal AchR), a folate binding protein (FBP), folate receptor alpha, ganglioside GD2, O-acetylated GD2 (OGD2), ganglioside GD3, glycoprotein 100 (gp100), glypican-3 (GPC3), G protein-coupled receptor class C group 5 member D (GPRC5D), Her2/neu (receptor tyrosine kinase erb-B2), Her3 (erb-B3), Her4 (erb-B4), erbB dimers, Human high molecular weight-melanoma-associated antigen (HMW-MAA), hepatitis B surface antigen, Human leukocyte antigen A1 (HLA-A1), Human leukocyte antigen A2 (HLA-A2), IL-22 receptor alpha (IL-22Rα), IL-13 receptor alpha 2 (IL-13Rα2), kinase insert domain receptor (kdr), kappa light chain, L1 cell adhesion molecule (L1-CAM), CE7 epitope of L1-CAM, Leucine Rich Repeat Containing 8 Family Member A (LRRC8A), Lewis Y, Melanoma-associated antigen (MAGE)-A1, MAGE-A3, MAGE-A6, MAGE-A10, mesothelin (MSLN), c-Met, murine cytomegalovirus (CMV), mucin 1 (MUC1), MUC16, natural killer group 2 member D (NKG2D) ligands, melan A (MART-1), neural cell adhesion molecule (NCAM), oncofetal antigen, Preferentially expressed antigen of melanoma (PRAME), progesterone receptor, a prostate specific antigen, prostate stem cell antigen (PSCA), prostate specific membrane antigen (PSMA), Receptor Tyrosine Kinase Like Orphan Receptor 1 (ROR1), survivin, Trophoblast glycoprotein (TPBG also known as 5T4), tumor-associated glycoprotein 72 (TAG72), Tyrosinase related protein 1 (TRP1, also known as TYRP1 or gp75), Tyrosinase related protein 2 (TRP2, also known as dopachrome tautomerase, dopachrome delta-isomerase or DCT), vascular endothelial growth factor receptor (VEGFR), vascular endothelial growth factor receptor 2 (VEGFR2), Wilms Tumor 1 (WT-1), a pathogen-specific or pathogen-expressed antigen, or an antigen associated with a universal tag, and/or biotinylated molecules, and/or molecules expressed by HIV, HCV, HBV or other pathogens.

21. The genetically engineered T cell of any of embodiments 15-20, wherein the extracellular region comprises a spacer, optionally wherein the spacer is operably linked between the binding domain and the transmembrane domain.

22. The genetically engineered T cell of embodiment 21, wherein the spacer comprises an immunoglobulin hinge region.

23. The genetically engineered T cell of embodiment 21 or embodiment 22, wherein the spacer comprises a C_{H}2 region and a C_{H}3 region.

24. The genetically engineered T cell of any of embodiments 15-23, wherein the intracellular region comprises an intracellular signaling domain.

25. The genetically engineered T cell of embodiment 24, wherein the intracellular signaling domain is or comprises an intracellular signaling domain of a CD3 chain, optionally a CD3-zeta (CD3ζ) chain, or a signaling portion thereof.

26. The genetically engineered T cell of embodiment 24 or embodiment 25, wherein the intracellular region comprises one or more costimulatory signaling domain(s).

27. The genetically engineered T cell of embodiment 26, wherein the one or more costimulatory signaling domain comprises an intracellular signaling domain of a CD28, a 4-1BB or an ICOS or a signaling portion thereof.

28. The chimeric antigen receptor of embodiment 26 or embodiment 27, wherein the costimulatory signaling region comprises an intracellular signaling domain of 4-1BB.

29. The genetically engineered T cell of any of embodiments 16-28, wherein the modified TGFBR2 locus encodes a recombinant receptor that comprises, from its N to C terminus in order: the extracellular binding domain, the spacer, the transmembrane domain and an intracellular signaling region.

30. The genetically engineered T cell of any of embodiments 1-10 and 13-29, wherein

the transgene sequence comprises in order a sequence of nucleotides encoding an extracellular binding domain, optionally an scFv; a spacer, optionally comprising a sequence from a human immunoglobulin hinge, optionally from IgG1, IgG2 or IgG4 or a modified version thereof, optionally further comprising a C_{H}2 region and/or a C_{H}3 region; and a transmembrane domain, optionally from human CD28; a costimulatory signaling domain, optionally from human 4-1BB; and an intracellular signaling region, optionally a CD3ζ chain or a portion thereof; and/or

the modified TGFBR2 locus comprises in order: a sequence of nucleotides encoding an extracellular binding domain, optionally an scFv; a spacer, optionally comprising a sequence from a human immunoglobulin hinge, optionally from IgG1, IgG2 or IgG4 or a modified version thereof, optionally further comprising a C_{H}2 region and/or a C_{H}3 region; and a transmembrane domain, optionally from human CD28; a costimulatory signaling domain, optionally from human 4-1BB; and an intracellular signaling region, optionally a CD3ζ chain or a portion thereof.

31. The genetically engineered T cell of any of embodiments 14-30, wherein the CAR is a multi-chain CAR.

32. The genetically engineered T cell of any of embodiments 1-30, wherein the transgene sequence comprises a sequence of nucleotides encoding at least one further protein.

33. The genetically engineered T cell of any of embodiments 1-32, wherein the transgene sequence comprises one or more multicistronic element(s).

34. The genetically engineered T cell of embodiment 33, wherein the one or more multicistronic element is positioned between the sequence of nucleotides encoding the CAR and the sequence of nucleotides encoding the at least one further protein.

35. The genetically engineered T cell of any of embodiments 32-34, wherein the at least one further protein is a surrogate marker, optionally wherein the surrogate marker is a truncated receptor, optionally wherein the truncated receptor lacks an intracellular signaling domain and/or is not capable of mediating intracellular signaling when bound by its ligand.

36. The genetically engineered T cell of embodiment 33, wherein the recombinant receptor is a recombinant TCR, and a multicistronic element is positioned between a sequence of nucleotides encoding the TCRα and a sequence of nucleotides encoding the TCRβ.

37. The genetically engineered T cell of embodiment 33, wherein the recombinant receptor is a multi-chain CAR, and a multicistronic element is positioned between a sequence of nucleotides encoding one chain of the multi-chain CAR and a sequence of nucleotides encoding another chain of the multi-chain CAR.

38. The genetically engineered T cell of any of embodiments 33-37, wherein the one or more multicistronic element(s) are upstream of the sequence of nucleotides encoding the recombinant receptor.

39. The genetically engineered T cell of any of embodiments 33-38, wherein the one or more multicistronic element is or comprises a ribosome skip sequence, optionally wherein the ribosome skip sequence is a T2A, a P2A, an E2A, or an F2A element.

40. The genetically engineered T cell of any of embodiments 1-39, wherein the modified TGFBR2 locus comprises the promoter and/or regulatory or control element of the endogenous TGFBR2 locus operably linked to control expression the nucleic acid sequence encoding the recombinant receptor.

41. The genetically engineered T cell of any of embodiments 1-39, wherein the modified locus comprises one or more heterologous regulatory or control element(s) operably linked to control expression of the nucleic acid sequence encoding the recombinant receptor.

42. The genetically engineered T cell of embodiment 41, wherein the one or more heterologous regulatory or control element comprises a heterologous promoter, an enhancer, an intron, a polyadenylation signal, a Kozak consensus sequence, a splice acceptor sequence or a splice donor sequence.

43. The genetically engineered T cell of embodiment 42, wherein the heterologous promoter is or comprises a human elongation factor 1 alpha (EF1α) promoter or an MND promoter or a variant thereof.

44. The genetically engineered T cell of any of embodiments 1-44, wherein the T cell is a primary T cell derived from a subject, optionally wherein the subject is a human.

45. The genetically engineered T cell of any of embodiments 1-44, wherein the T cell is a CD8+ T cell or subtypes thereof.

46. The genetically engineered T cell of any of embodiments 1-44, wherein the T cell is a CD4+ T cell or subtypes thereof.

47. The genetically engineered T cell of any of embodiments 1-46, wherein the T cell is derived from a multipotent or pluripotent cell, which optionally is an iPSC.

48. A polynucleotide, comprising:

(a) a nucleic acid sequence encoding a recombinant receptor or a portion thereof; and

(b) one or more homology arm(s) linked to the nucleic acid sequence, wherein the one or more homology arm(s) comprise a sequence homologous to one or more region(s) of an open reading frame of a transforming growth factor-beta receptor type-2 (TGFBR2) locus.

49. The polynucleotide of embodiment 48, wherein the recombinant receptor or a portion thereof is encoded by a modified TGFBR2 locus comprising the nucleic acid sequence encoding the recombinant receptor or a portion thereof when the recombinant receptor is expressed from a cell introduced with the polynucleotide.

50. The polynucleotide of embodiment 48 or embodiment 49, wherein the nucleic acid sequence in (a) is a sequence that is exogenous or heterologous to an open reading frame of the endogenous genomic TGFBR2 locus a T cell, optionally a human T cell.

51. The polynucleotide of any of embodiments 48-50, wherein the one or more homology arm(s) comprise at least one intron or at least one exon of the open reading frame of the TGFBR2 locus.

52. The polynucleotide of any of embodiments 48-51, wherein the modified TGFBR2 locus does not encode a functional TGFBRII polypeptide, in a cell introduced with the polynucleotide.

53. The polynucleotide of any of embodiments 48-52, wherein the modified TGFBR2 locus does not encode a TGFBRII polypeptide or the expression of TGFBRII polypeptide is eliminated, in a cell introduced with the polynucleotide.

54. The polynucleotide of any of embodiments 48-52, wherein the modified TGFBR2 locus does not encode a full length TGFBRII polypeptide or encodes a partial TGFBRII polypeptide, in a cell introduced with the polynucleotide.

55. The polynucleotide of any of embodiments 48-52 and 54, wherein the modified TGFBR2 locus encodes a dominant negative TGFBRII polypeptide, in a cell introduced with the polynucleotide.

56. The polynucleotide of any of embodiments 48-52, 54 and 55, wherein the encoded TGFBRII polypeptide in a cell introduced with the polynucleotide comprises an amino acid sequence corresponding to residues 22-191 of SEQ ID NO:59 or residues 22-216 of SEQ ID NO:60, or a sequence that exhibits at least 85%, 86%, 87%, 88%, 89%, 90%, 91%, 92%, 93%, 94%, 95%, 96%, 97%, 98%, 99% or more sequence identity to an amino acid sequence corresponding to residues 22-191 of SEQ ID NO:59 or residues 22-216 of SEQ ID NO:60, or a fragment thereof.

57. The polynucleotide of any of embodiments 48-52 and 54-56, wherein the nucleic acid sequence in (a) is in-frame with one or more exons of the open reading frame of the TGFBR2 locus comprised in the one or more homology arm(s).

58. The polynucleotide of any of embodiments 48-57, wherein the one or more region(s) of the open reading frame is or comprises sequences that are downstream of exon 1 of the open reading frame of the endogenous TGFBR2 locus.

59. The polynucleotide of any of embodiments 48-58, wherein the one or more region(s) of the open reading frame is or comprises sequences that includes at least a portion of exon 4 or downstream of exon 4 of the open reading frame of the TGFBR2 locus.

60. The polynucleotide of any of embodiments 48-59, wherein the one or more homology arm comprises a 5′ homology arm and a 3′ homology arm.

61. The polynucleotide of embodiment 60, wherein the polynucleotide comprises the structure [5′ homology arm]-[nucleic acid sequence of (a)]-[3′ homology arm].

62. The polynucleotide of embodiment 60 or embodiment 61, wherein the 5′ homology arm and the 3′ homology arm independently are from at or about 50 to at or about 2000 nucleotides, from at or about 100 to at or about 1000 nucleotides, from at or about 100 to at or about 750 nucleotides, from at or about 100 to at or about 600 nucleotides, from at or about 100 to at or about 400 nucleotides, from at or about 100 to at or about 300 nucleotides, from at or about 100 to at or about 200 nucleotides, from at or about 200 to at or about 1000 nucleotides, from at or about 200 to at or about 750 nucleotides, from at or about 200 to at or about 600 nucleotides, from at or about 200 to at or about 400 nucleotides, from at or about 200 to at or about 300 nucleotides, from at or about 300 to at or about 1000 nucleotides, from at or about 300 to at or about 750 nucleotides, from at or about 300 to at or about 600 nucleotides, from at or about 300 to at or about 400 nucleotides, from at or about 400 to at or about 1000 nucleotides, from at or about 400 to at or about 750 nucleotides, from at or about 400 to at or about 600 nucleotides, from at or about 600 to at or about 1000 nucleotides, from at or about 600 to at or about 750 nucleotides or from at or about 750 to at or about 1000 nucleotides in length.

63. The polynucleotide of any of embodiments 60-62, wherein the 5′ homology arm and the 3′ homology arm independently are at or about 200, 300, 400, 500, 600, 700 or 800 nucleotides in length, or any value between any of the foregoing.

64. The polynucleotide of any of embodiments 60-63, wherein the 5′ homology arm and the 3′ homology arm independently are greater than at or about 300 nucleotides in length, optionally wherein the 5′ homology arm and the 3′ homology arm independently are at or about 400, 500 or 600 nucleotides in length, or any value between any of the foregoing.

65. The polynucleotide of any of embodiments 60-64, wherein the 5′ homology arm comprises the sequence set forth in SEQ ID NOS: 69-71 or a sequence that exhibits at least 85%, 86%, 87%, 88%, 89%, 90%, 91%, 92%, 93%, 94%, 95%, 96%, 97%, 98%, 99% or more sequence identity to SEQ ID NOS: 69-71 or a partial sequence thereof.

66. The polynucleotide of any of embodiments 60-65, wherein the 3′ homology arm comprises the sequence set forth in SEQ ID NO:72, or a sequence that exhibits at least 85%, 86%, 87%, 88%, 89%, 90%, 91%, 92%, 93%, 94%, 95%, 96%, 97%, 98%, 99% or more sequence identity to SEQ ID NO:72 or a partial sequence thereof.

67. The polynucleotide of any of embodiments 48-66, wherein the encoded recombinant receptor is or comprises recombinant T cell receptor (TCR).

68. The polynucleotide of any of embodiments 48-67, wherein the encoded recombinant receptor is a recombinant TCR and the nucleic acid sequence in (a) encodes a TCR alpha (TCRα) chain, a TCR beta (TCRβ) chain or both.

69. The polynucleotide of any of embodiments 48-66, wherein the encoded recombinant receptor is or comprises a functional non-T cell receptor (non-TCR) antigen receptor.

70. The polynucleotide of any of embodiments 48-66 and 69, wherein the encoded recombinant receptor is a chimeric antigen receptor (CAR).

71. The polynucleotide of embodiment 70, wherein the CAR comprises an extracellular region, a transmembrane domain, and an intracellular region.

72. The polynucleotide of any of embodiments 71, wherein the extracellular region comprises a binding domain.

73. The polynucleotide of embodiment 72, wherein the binding domain is or comprises an antibody or an antigen-binding fragment thereof.

74. The polynucleotide of embodiment 72 and embodiment 73, wherein the binding domain is capable of binding to a target antigen that is associated with, specific to, or expressed on a cell or tissue of a disease, disorder or condition.

75. The polynucleotide of embodiment 74, wherein the target antigen is a tumor antigen.

76. The polynucleotide of embodiment 74 or embodiment 75, wherein the target antigen is selected from among αvβ6 integrin (avb6 integrin), B cell maturation antigen (BCMA), B7-H3, B7-H6, carbonic anhydrase 9 (CA9, also known as CAIX or G250), a cancer-testis antigen, cancer/testis antigen 1B (CTAG, also known as NY-ESO-1 and LAGE-2), carcinoembryonic antigen (CEA), a cyclin, cyclin A2, C—C Motif Chemokine Ligand 1 (CCL-1), CD19, CD20, CD22, CD23, CD24, CD30, CD33, CD38, CD44, CD44v6, CD44v7/8, CD123, CD133, CD138, CD171, chondroitin sulfate proteoglycan 4 (CSPG4), epidermal growth factor protein (EGFR), type III epidermal growth factor receptor mutation (EGFR vIII), epithelial glycoprotein 2 (EPG-2), epithelial glycoprotein 40 (EPG-40), ephrinB2, ephrin receptor A2 (EPHa2), estrogen receptor, Fc receptor like 5 (FCRL5; also known as Fc receptor homolog 5 or FCRH5), fetal acetylcholine receptor (fetal AchR), a folate binding protein (FBP), folate receptor alpha, ganglioside GD2, O-acetylated GD2 (OGD2), ganglioside GD3, glycoprotein 100 (gp100), glypican-3 (GPC3), G protein-coupled receptor class C group 5 member D (GPRC5D), Her2/neu (receptor tyrosine kinase erb-B2), Her3 (erb-B3), Her4 (erb-B4), erbB dimers, Human high molecular weight-melanoma-associated antigen (HMW-MAA), hepatitis B surface antigen, Human leukocyte antigen A1 (HLA-A1), Human leukocyte antigen A2 (HLA-A2), IL-22 receptor alpha (IL-22Rα), IL-13 receptor alpha 2 (IL-13Rα2), kinase insert domain receptor (kdr), kappa light chain, L1 cell adhesion molecule (L1-CAM), CE7 epitope of L1-CAM, Leucine Rich Repeat Containing 8 Family Member A (LRRC8A), Lewis Y, Melanoma-associated antigen (MAGE)-A1, MAGE-A3, MAGE-A6, MAGE-A10, mesothelin (MSLN), c-Met, murine cytomegalovirus (CMV), mucin 1 (MUC1), MUC16, natural killer group 2 member D (NKG2D) ligands, melan A (MART-1), neural cell adhesion molecule (NCAM), oncofetal antigen, Preferentially expressed antigen of melanoma (PRAME), progesterone receptor, a prostate specific antigen, prostate stem cell antigen (PSCA), prostate specific membrane antigen (PSMA), Receptor Tyrosine Kinase Like Orphan Receptor 1 (ROR1), survivin, Trophoblast glycoprotein (TPBG also known as 5T4), tumor-associated glycoprotein 72 (TAG72), Tyrosinase related protein 1 (TRP1, also known as TYRP1 or gp75), Tyrosinase related protein 2 (TRP2, also known as dopachrome tautomerase, dopachrome delta-isomerase or DCT), vascular endothelial growth factor receptor (VEGFR), vascular endothelial growth factor receptor 2 (VEGFR2), Wilms Tumor 1 (WT-1), a pathogen-specific or pathogen-expressed antigen, or an antigen associated with a universal tag, and/or biotinylated molecules, and/or molecules expressed by HIV, HCV, HBV or other pathogens.

77. The polynucleotide of any of embodiments 71-76, wherein the extracellular region comprises a spacer, optionally wherein the spacer is operably linked between the binding domain and the transmembrane domain.

78. The polynucleotide of embodiment 77, wherein the spacer comprises an immunoglobulin hinge region.

79. The polynucleotide of embodiment 77 or embodiment 78, wherein the spacer comprises a C_{H}2 region and a C_{H}3 region.

80. The polynucleotide of any of embodiments 71-79, wherein the intracellular region comprises an intracellular signaling domain.

81. The polynucleotide of any of embodiments 71-80, wherein the intracellular signaling domain is or comprises an intracellular signaling domain of a CD3 chain, optionally a CD3-zeta (CD3) chain, or a signaling portion thereof.

82. The polynucleotide of any of embodiments 71-81, wherein the intracellular region comprises one or more costimulatory signaling domain(s).

83. The polynucleotide of embodiment 82, wherein the one or more costimulatory signaling domain comprises an intracellular signaling domain of a CD28, a 4-1BB or an ICOS or a signaling portion thereof.

84. The polynucleotide of embodiment 82 or embodiment 83, wherein the costimulatory signaling region comprises an intracellular signaling domain of 4-1BB.

85. The polynucleotide of any of embodiments 72-84, wherein the modified TGFBR2 locus encodes a recombinant receptor that comprises, from its N to C terminus in order: the extracellular binding domain, the spacer, the transmembrane domain and an intracellular signaling region.

86. The polynucleotide of any of embodiments 48-66 and 68-85, wherein the transgene sequence comprises in order a sequence of nucleotides encoding an extracellular binding domain, optionally an scFv; a spacer, optionally comprising a sequence from a human immunoglobulin hinge, optionally from IgG1, IgG2 or IgG4 or a modified version thereof, optionally further comprising a C_{H}2 region and/or a C_{H}3 region; and a transmembrane domain, optionally from human CD28; a costimulatory signaling domain, optionally from human 4-1BB; and an intracellular signaling region, optionally a CD3ζ chain or a portion thereof.

87. The polynucleotide of any of embodiments 70-86, wherein the CAR is a multi-chain CAR.

88. The polynucleotide of any of embodiments 48-87, wherein the nucleic acid sequence in (a) comprises a sequence of nucleotides encoding at least one further protein.

89. The polynucleotide of any of embodiments 48-88, wherein the nucleic acid sequence in (a) comprises one or more multicistronic element(s).

90. The polynucleotide of embodiment 89, wherein the one or more multicistronic element is positioned between the sequence of nucleotides encoding the CAR and the sequence of nucleotides encoding the at least one further protein.

91. The polynucleotide of any of embodiments 88-90, wherein the at least one further protein is a surrogate marker, optionally wherein the surrogate marker is a truncated receptor, optionally wherein the truncated receptor lacks an intracellular signaling domain and/or is not capable of mediating intracellular signaling when bound by its ligand.

92. The polynucleotide of embodiment 89, wherein the recombinant receptor is a recombinant TCR, and a multicistronic element is positioned between a sequence of nucleotides encoding the TCRα and a sequence of nucleotides encoding the TCRβ.

93. The polynucleotide of embodiment 89, wherein the recombinant receptor is a multi-chain CAR, and a multicistronic element is positioned between a sequence of nucleotides encoding one chain of the multi-chain CAR and a sequence of nucleotides encoding another chain of the multi-chain CAR.

94. The polynucleotide of any of embodiments 89-93, wherein the one or more multicistronic element(s) are upstream of the sequence of nucleotides encoding the recombinant receptor.

95. The polynucleotide of any of embodiments 89-94, wherein the one or more multicistronic element is or comprises a ribosome skip sequence, optionally wherein the ribosome skip sequence is a T2A, a P2A, an E2A, or an F2A element.

96. The polynucleotide of any of embodiments 48-95, wherein the nucleic acid sequence of (a) comprises one or more heterologous or regulatory control element(s) operably linked to control expression of the recombinant receptor when expressed from a cell introduced with the polynucleotide.

97. The polynucleotide of embodiment 96, wherein the one or more heterologous regulatory or control element comprises a heterologous promoter, an enhancer, an intron, a polyadenylation signal, a Kozak consensus sequence, a splice acceptor sequence and/or a splice donor sequence.

98. The polynucleotide of embodiment 97, wherein the heterologous promoter is or comprises a human elongation factor 1 alpha (EF1α) promoter or an MND promoter or a variant thereof.

99. The polynucleotide of any of embodiments 48-98, wherein the polynucleotide is comprised in a viral vector.

100. The polynucleotide of embodiment 99, wherein the viral vector is an AAV vector.

101. The polynucleotide of embodiment 100, wherein the AAV vector is selected from among AAV1, AAV2, AAV3, AAV4, AAV5, AAV6, AAV7 or AAV8 vector.

102. The polynucleotide of embodiment 100 or embodiment 101, wherein the AAV vector is an AAV2 or AAV6 vector.

103. The polynucleotide of embodiment 99, wherein the viral vector is a retroviral vector, optionally a lentiviral vector.

104. The polynucleotide of any of embodiments 48-98, that is a linear polynucleotide, optionally a double-stranded polynucleotide or a single-stranded polynucleotide.

105. The polynucleotide of any of embodiments 48-104, wherein the polynucleotide is at least at or about 2500, 2750, 3000, 3250, 3500, 3750, 4000, 4250, 4500, 4760, 5000, 5250, 5500, 5750, 6000, 7000, 7500, 8000, 9000 or 10000 nucleotides in length, or any value between any of the foregoing.

106. The polynucleotide of any of embodiments 48-105, wherein the polynucleotide is between at or about 2500 and at or about 5000 nucleotides, at or about 3500 and at or about 4500 nucleotides, or at or about 3750 nucleotides and at or about 4250 nucleotides in length.

107. A method of producing a genetically engineered T cell, the method comprising introducing the polynucleotide of any of embodiments 48-106 into a T cell comprising a genetic disruption at a TGFBR2 locus.

108. A method of producing a genetically engineered T cell, the method comprising:

(a) introducing, into a T cell, one or more agent(s) capable of inducing a genetic disruption at a target site within an endogenous TGFBR2 locus of the T cell; and

(b) introducing the polynucleotide of any of embodiments 48-106 into a T cell comprising a genetic disruption at a TGFBR2 locus, wherein the method produces a modified TGFBR2 locus, said modified TGFBR2 locus comprising a nucleic acid sequence encoding the recombinant receptor or a portion thereof.

109. The method of embodiment 108, wherein the nucleic acid sequence encoding a recombinant receptor or a portion thereof is integrated within the endogenous TGFBR2 locus via homology directed repair (HDR).

110. A method of producing a genetically engineered T cell, the method comprising introducing, into a T cell, a polynucleotide comprising a nucleic acid sequence encoding a recombinant receptor or a portion thereof, said T cell having a genetic disruption within a TGFBR2 locus of the T cell, wherein the nucleic acid sequence encoding the recombinant receptor or a portion thereof is integrated within the endogenous TGFBR2 locus via homology directed repair (HDR).

111. The method of embodiment 107 or embodiment 110, wherein the genetic disruption is carried out by introducing, into a T cell, one or more agent(s) capable of inducing a genetic disruption at a target site within an endogenous TGFBR2 locus of the T cell.

112. The method of any of embodiments 107-111, wherein the method produces a modified TGFBR2 locus, said modified TGFBR2 locus comprising a nucleic acid sequence encoding a recombinant receptor or a portion thereof.

113. The method of any of embodiments 110-112, wherein the polynucleotide further comprises one or more homology arm(s) linked to the nucleic acid sequence, wherein the one or more homology arm(s) comprise a sequence homologous to one or more region(s) of an open reading frame of a transforming growth factor-beta receptor type-2 (TGFBR2) locus.

114. The method of any of embodiments 110-113, wherein the modified TGFBR2 locus does not encode a functional TGFBRII polypeptide, in a cell generated by the method.

115. The method of any of embodiments 110-114, wherein the modified TGFBR2 locus does not encode a TGFBRII polypeptide or the expression of TGFBRII polypeptide is eliminated, in a cell generated by the method.

116. The method of any of embodiments 110-114, wherein the modified TGFBR2 locus does not encode a full length TGFBRII polypeptide or encodes a partial TGFBRII polypeptide, in a cell generated by the method.

117. The method of any of embodiments 110-114 and 116, wherein the modified TGFBR2 locus encodes a dominant negative TGFBRII polypeptide, in a cell generated by the method.

118. The method of any of embodiments 113-117, wherein the one or more homology arm comprises a 5′ homology arm and a 3′ homology arm.

119. The method of embodiment 118, wherein the polynucleotide comprises the structure [5′ homology arm]-[the nucleic acid sequence encoding a recombinant receptor or a portion thereof]-[3′ homology arm].

120. The method of embodiment 118 or embodiment 119, wherein the 5′ homology arm and the 3′ homology arm independently are from at or about 50 to at or about 2000 nucleotides, from at or about 100 to at or about 1000 nucleotides, from at or about 100 to at or about 750 nucleotides, from at or about 100 to at or about 600 nucleotides, from at or about 100 to at or about 400 nucleotides, from at or about 100 to at or about 300 nucleotides, from at or about 100 to at or about 200 nucleotides, from at or about 200 to at or about 1000 nucleotides, from at or about 200 to at or about 750 nucleotides, from at or about 200 to at or about 600 nucleotides, from at or about 200 to at or about 400 nucleotides, from at or about 200 to at or about 300 nucleotides, from at or about 300 to at or about 1000 nucleotides, from at or about 300 to at or about 750 nucleotides, from at or about 300 to at or about 600 nucleotides, from at or about 300 to at or about 400 nucleotides, from at or about 400 to at or about 1000 nucleotides, from at or about 400 to at or about 750 nucleotides, from at or about 400 to at or about 600 nucleotides, from at or about 600 to at or about 1000 nucleotides, from at or about 600 to at or about 750 nucleotides or from at or about 750 to at or about 1000 nucleotides in length.

121. The method of any of embodiments 118-120, wherein the 5′ homology arm and the 3′ homology arm independently are at or about 200, 300, 400, 500, 600, 700 or 800 nucleotides in length, or any value between any of the foregoing.

122. The method of any of embodiments 118-121, wherein the 5′ homology arm and the 3′ homology arm independently are greater than at or about 300 nucleotides in length, optionally wherein the 5′ homology arm and the 3′ homology arm independently are at or about 400, 500 or 600 nucleotides in length, or any value between any of the foregoing.

123. The method of any of embodiments 118-122, wherein the 5′ homology arm comprises the sequence set forth in SEQ ID NOS: 69-71 or a sequence that exhibits at least 85%, 86%, 87%, 88%, 89%, 90%, 91%, 92%, 93%, 94%, 95%, 96%, 97%, 98%, 99% or more sequence identity to SEQ ID NOS: 69-71 or a partial sequence thereof.

124. The method of any of embodiments 118-123, wherein the 3′ homology arm comprises the sequence set forth in SEQ ID NO:72, or a sequence that exhibits at least 85%, 86%, 87%, 88%, 89%, 90%, 91%, 92%, 93%, 94%, 95%, 96%, 97%, 98%, 99% or more sequence identity to SEQ ID NO:72 or a partial sequence thereof.

125. The method of any of embodiments 110-124, wherein the encoded recombinant receptor is or comprises recombinant T cell receptor (TCR).

126. The method of any of embodiments 110-124, wherein the encoded recombinant receptor is a chimeric antigen receptor (CAR).

127. The method of any of embodiments 108 and 111-126, wherein the one or more agent(s) capable of inducing a genetic disruption comprises a DNA binding protein or DNA-binding nucleic acid that specifically binds to or hybridizes to the target site, a fusion protein comprising a DNA-targeting protein and a nuclease, or an RNA-guided nuclease, optionally wherein the one or more agent(s) comprises a zinc finger nuclease (ZFN), a TAL-effector nuclease (TALEN), or and a CRISPR-Cas9 combination that specifically binds to, recognizes, or hybridizes to the target site.

128. The method of any of embodiments 108 and 111-127, wherein the each of the one or more agent(s) comprises a guide RNA (gRNA) having a targeting domain that is complementary to the at least one target site.

129. The method of embodiment 128, wherein the one or more agent(s) is introduced as a ribonucleoprotein (RNP) complex comprising the gRNA and a Cas9 protein.

130. The method of embodiment 129, wherein the RNP is introduced via electroporation, particle gun, calcium phosphate transfection, cell compression or squeezing, optionally via electroporation.

131. The method of embodiment 129 or embodiment 130, wherein the concentration of the RNP is from at or about 1 μM to at or about 5 μM, optionally wherein the concentration of the RNP is at or about 2 μM.

132. The method of any of embodiments 128-131, wherein the gRNA has a targeting domain sequence of GUGGAUGACCUGGCUAACAG (SEQ ID NO:73).

133. The method of any of embodiments 107-132, wherein the T cell is a primary T cell derived from a subject, optionally wherein the subject is a human.

134. The method of any of embodiments 107-133, wherein the T cell is a CD8+ T cell or subtypes thereof.

135. The method of any of embodiments 107-133, wherein the T cell is a CD4+ T cell or subtypes thereof.

136. The method of any of embodiments 107-135, wherein the T cell is derived from a multipotent or pluripotent cell, which optionally is an iPSC.

137. The method of any of embodiments 110-136, wherein the polynucleotide is comprised in a viral vector.

138. The method of embodiment 137, wherein the viral vector is an AAV vector.

139. The method of embodiment 138, wherein the AAV vector is selected from among AAV1, AAV2, AAV3, AAV4, AAV5, AAV6, AAV7 or AAV8 vector.

140. The method of embodiment 138 or embodiment 139, wherein the AAV vector is an AAV2 or AAV6 vector.

141. The method of embodiment 137, wherein the viral vector is a retroviral vector, optionally a lentiviral vector.

142. The method of any of embodiments 110-136, wherein the polynucleotide is a linear polynucleotide, optionally a double-stranded polynucleotide or a single-stranded polynucleotide.

143. The method of any of embodiments 108 and 111-142, wherein the one or more agent(s) and the polynucleotide are introduced simultaneously or sequentially, in any order.

144. The method of any of embodiments 108 and 111-143, wherein the polynucleotide is introduced after the introduction of the one or more agent(s).

145. The method of embodiment 144, wherein the polynucleotide is introduced immediately after, or within about 30 seconds, 1 minute, 2 minutes, 3 minutes, 4 minutes, 5 minutes, 6 minutes, 6 minutes, 8 minutes, 9 minutes, 10 minutes, 15 minutes, 20 minutes, 30 minutes, 40 minutes, 50 minutes, 60 minutes, 90 minutes, 2 hours, 3 hours or 4 hours after the introduction of the agent.

146. The method of any of embodiments 108 and 111-141, wherein prior to the introducing of the one or more agent, the method comprises incubating the cells, in vitro with a stimulatory agent(s) under conditions to stimulate or activate the one or more immune cells.

147. The method of embodiment 146, wherein the stimulatory agent(s) comprises and anti-CD3 and/or anti-CD28 antibodies, optionally anti-CD3/anti-CD28 beads, optionally wherein the bead to cell ratio is or is about 1:1.

148. The method of embodiment 146 or embodiment 147, comprising removing the stimulatory agent(s) from the one or more immune cells prior to the introducing with the one or more agents.

149. The method of any of embodiments 108 and 111-148, wherein the method further comprises incubating the cells prior to, during or subsequent to the introducing of the one or more agents and/or the introducing of the template polynucleotide with one or more recombinant cytokines, optionally wherein the one or more recombinant cytokines are selected from the group consisting of IL-2, IL-7, and IL-15.

150. The method of embodiment 149, wherein the one or more recombinant cytokine is added at a concentration selected from a concentration of IL-2 from at or about 10 U/mL to at or about 200 U/mL, optionally at or about 50 IU/mL to at or about 100 U/mL; IL-7 at a concentration of 0.5 ng/mL to 50 ng/mL, optionally at or about 5 ng/mL to at or about 10 ng/mL and/or IL-15 at a concentration of 0.1 ng/mL to 20 ng/mL, optionally at or about 0.5 ng/mL to at or about 5 ng/mL.

151. The method of embodiment 149 or embodiment 150, wherein the incubation is carried out subsequent to the introducing of the one or more agents and the introducing of the template polynucleotide for up to or approximately 24 hours, 36 hours, 48 hours, 3, 4, 5, 6, 7, 8, 9, 10, 11, 12, 13, 14, 15, 16, 17, 18, 19, 20 or 21 days, optionally up to or about 7 days.

152. The method of any of embodiments 107-151, wherein at least or greater than 35%, 40%, 45%, 50%, 55%, 60%, 65%, 70%, 75%, 80%, or 90% of the cells in a plurality of engineered cells generated by the method comprise a genetic disruption of at least one target site within a TGFBR2 locus.

153. The method of any of embodiments 107-152, wherein at least or greater than 35%, 40%, 45%, 50%, 55%, 60%, 65%, 70%, 75%, 80%, or 90% of the cells in a plurality of engineered cells generated by the method express the recombinant receptor or antigen-binding fragment thereof.

154. An engineered T cell or a plurality of engineered T cells generated using the method of any of embodiments 107-153.

155. A composition, comprising the engineered T cell any of embodiments 1-47 and 154.

156. A composition, comprising a plurality of the engineered T cell any of embodiments 1-47 and 154.

157. The composition of embodiment 155 or embodiment 156, wherein the composition comprises CD4+ and/or CD8+ T cells.

158. The composition of any of embodiments 155-157, wherein the composition comprises CD4+ and CD8+ T cells and the ratio of CD4+ to CD8+ T cells is from or from about 1:3 to 3:1, optionally 1:1.

159. The composition of any of embodiments 155-158, wherein cells expressing the recombinant receptor make up at least 30%, 40%, 50%, 60%, 70%, 80%, 90%, 91%, 92%, 93%, 94%, 95%, 96%, 97%, 98%, 99%, or more of the total cells in the composition or of the total CD4+ or CD8+ cells in the composition.

160. A method of treatment comprising administering the engineered cell, plurality of engineered cells or composition of any of embodiments 1-47 and 154-159 to a subject having a disease or disorder.

161. Use of the engineered cell, plurality of engineered cells or composition of any of embodiments 1-47 and 154-159 for the treatment of a disease or disorder.

162. Use of the engineered cell, plurality of engineered cells or composition of any of embodiments 1-47 and 154-159 in the manufacture of a medicament for treating a disease or disorder.

163. The engineered cell, plurality of engineered cells or composition of any of embodiments 1-47 and 154-159 for use in the treatment of a disease or disorder.

164. The method, use or the engineered cell, plurality of engineered cells or composition for use of any of embodiments 160-163, wherein the disease or disorder is a cancer or a tumor.

165. The method, use or the engineered cell, plurality of engineered cells or composition for use of embodiment 164, wherein the cancer or the tumor is a hematologic malignancy, optionally a lymphoma, a leukemia, or a plasma cell malignancy.

166. The method, use or the engineered cell, plurality of engineered cells or composition for use of embodiment 164 or embodiment 165, wherein the cancer is a lymphoma and the lymphoma is Burkitt's lymphoma, non-Hodgkin's lymphoma (NHL), Hodgkin's lymphoma, Waldenstrom macroglobulinemia, follicular lymphoma, small non-cleaved cell lymphoma, mucosa-associated lymphatic tissue lymphoma (MALT), marginal zone lymphoma, splenic lymphoma, nodal monocytoid B cell lymphoma, immunoblastic lymphoma, large cell lymphoma, diffuse mixed cell lymphoma, pulmonary B cell angiocentric lymphoma, small lymphocytic lymphoma, primary mediastinal B cell lymphoma, lymphoplasmacytic lymphoma (LPL), or mantle cell lymphoma (MCL).

167. The method, use or the engineered cell, plurality of engineered cells or composition for use of embodiment 164 or embodiment 165, wherein the cancer is a leukemia and the leukemia is chronic lymphocytic leukemia (CLL), plasma cell leukemia or acute lymphocytic leukemia (ALL).

168. The method, use or the engineered cell, plurality of engineered cells or composition for use of embodiment 164 or embodiment 165, wherein the cancer is a plasma cell malignancy and the plasma cell malignancy is multiple myeloma (MM).

169. The method, use or the engineered cell, plurality of engineered cells or composition for use of embodiment 164, wherein the tumor is a solid tumor.

170. The method, use or the engineered cell, plurality of engineered cells or composition for use of embodiment 169, wherein the solid tumor is a non-small cell lung cancer (NSCLC) or a head and neck squamous cell carcinoma (HNSCC).

171. A kit comprising:

one or more agent(s) capable of inducing a genetic disruption at a target site within a TGFBR2 locus; and

the polynucleotide of any of embodiments 48-106.

172. A kit, comprising:

one or more agent(s) capable of inducing a genetic disruption at a target site within a TGFBR2 locus; and

a polynucleotide comprising a nucleic acid sequence encoding recombinant receptor or a portion thereof, wherein the transgene encoding the recombinant receptor or antigen-binding fragment or chain thereof is targeted for integration at or near the target site via homology directed repair (HDR); and

instructions for carrying out the method of any of embodiments 107-153.

### IX. EXAMPLES

The following examples are included for illustrative purposes only and are not intended to limit the scope of the invention.

### Example 1 Generation and In Vivo Assessment of Engineered T Cells Expressing a Chimeric Antigen Receptor (CAR) with a Knockout (KO) or Dominant Negative (DN) Transforming Growth Factor Beta Receptor 2 (TGFBR2)

Human T cells were engineered to express an exemplary chimeric antigen receptor (CAR) that specifically binds an antigen associated with a tumor, and also were modified by genetic disruption to knock-out (KO) the transforming growth factor beta receptor 2 (TGFBR2) locus or by expressing a dominant negative transforming growth factor beta receptor II (DN-TGFBRII). DN-TGFBRII, which lacks the protein kinase domain of the receptor, was used as an alternative method to interfere with TGF beta (TGFβ) signaling, as the expression of DN-TGFBRII competes with the wild-type TGFBRII for TGFβ binding and forms a non-functional receptor complex. The engineered T cells were administered to a mouse tumor model with tumor cells that express the antigen, and monitored for anti-tumor activity.

### A. Generation of TGFBR2 KO and DN T Cells Expressing an Exemplary CAR

Primary human CD4+ and CD8+ T cells were isolated by immunoaffinity-based selection from human peripheral blood mononuclear cells (PBMCs) obtained from healthy donors. The resulting CD4+ and CD8+ cells (at 1:1 ratio) were stimulated by culturing with an anti-CD3/anti-CD28 reagent.

Lentiviral preparations were prepared for transduction of stimulated cells. An exemplary lentiviral vector for transduction of a chimeric antigen receptor (CAR) contained nucleic acid sequences encoding an exemplary anti-ROR1 CAR containing an scFv antigen-binding domain derived from the variable heavy and light chain of a chimeric rabbit/human IgG1 antibody designated R12 (see, e.g., Yang et al. (2011) PloS ONE, 6:e21018; U.S. Patent Application No. US 2013/0251642). The encoded CAR also included an immunoglobulin-derived spacer, a transmembrane domain, a costimulatory region, and a CD3ζ signaling domain. For transduction of a DN-TGFBRII with the CAR, the lentiviral construct contained nucleic acid sequences encoding a mature form of the dominant negative TGFBRII sequence corresponding to residues 22-191 of the TGFBR2 sequence set forth in SEQ ID NO:59) separated from the sequences encoding the anti-ROR1 CAR by a sequence encoding a T2A ribosome skip element. The nucleic acid sequence encoding the CAR (LV), or the CAR and DN-TGFBRII (LV+DN), was incorporated into an exemplary HIV-1 derived lentiviral vector. Pseudotyped lentiviral vector particles were produced by standard procedures by transiently transfecting HEK-293T cells with the resulting vectors, helper plasmids (containing gagpol plasmids and rev plasmid), and a pseudotyping plasmid and used to transduce cells.

At 24 hours, the cells were transduced with the lentiviral preparations, or were mock transduced as control (mock). For cells transduced with the anti-ROR1 (R12) CAR-encoding lentiviral preparation (not containing the DN-TGFBRII), cells also were engineered to knockout the endogenous TGFBR2 locus (LV+KO). The anti-CD3/anti-CD28 reagent was removed 72 hours after stimulation, and the stimulated cells were electroporated with 2.2 μM of ribonucleoprotein (RNP) complexes containing TGFBR2-targeting gRNA (containing the sequence GUGGAUGACCUGGCUAACAG (SEQ ID NO:73), which targets a genetic disruption within exon 4 of the endogenous TGFBR2 sequence (exon numbering based on isoform 1 as set forth in Table 1 herein)) and Streptococcus pyogenes Cas9, for knockout of the endogenous TGFBR2 gene (LV+KO or mock KO control), or electroporated without any RNP complexes (LV only or LV+DN). Electroporated cells were cultured for approximately 7 days before cryopreservation. Cells transduced with the R12 CAR-encoding lentivirus electroporated without RNPs (LV) and mock treated cells electroporated with RNPs (mock KO) were assessed as controls.

### B. Assessment of In Vivo Anti-Tumor Activity

The anti-tumor effects of exemplary engineered CAR-expressing primary human T cells with a knockout of TGFBR2 or expressing a DN-TGFBRII were assessed by monitoring tumors following adoptive transfer of cells into a tumor-bearing mouse xenograft model. NOD.Cg.Prkd^{scid}IL2rg^{tm1Wil}/SzJ (NSG) mice were each injected subcutaneously with approximately 5×10⁶ H1975 non-small cell lung cancer cells. On day 24 following tumor engraftment, the tumor volume was measured. Prior to CAR-expressing T cell administration, the mean tumor volume was approximately 190 mm³, with a range between 83 and 302 mm³.

Eight (8) mice in each group received a single intravenous (i.v.) injection of one of the engineered primary T cell compositions generated from one of two independent human donors (Donor 1, Donor 2), as follows: (1) engineered T cells expressing the anti-ROR1 CAR R12 by lentiviral delivery (LV only), (2) engineered T cells expressing the anti-ROR1 CAR R12 by lentiviral delivery and TGFBR2 knockout (LV+KO), or (3) engineered T cells expressing the anti-ROR1 CAR R12 and DN-TGFBRII by lentiviral delivery (LV+DN). The different groups of engineered T cells were each administered at a dose of 1×10⁶ cells (low dose) or 3×10⁶ cells (high dose). As a control, mice were administered 3×10⁶ mock treated cells (mock KO) or were untreated (tumor only). Tumor-free survival and tumor volume were assessed over approximately 120 days.

Anti-tumor activity of the adoptively transferred anti-ROR1 CAR⁺ T cells was monitored by determining the tumor volume every 3 to 6 days post administration. As shown in FIGS. 1A and 1C (group; Donor 1 and 2, respectively) and FIGS. 1B and 1D (individual mice; Donor 1 and 2, respectively), administration of anti-ROR1 CAR-expressing cells with a knockout of the TGFBR2 gene (KO) resulted in greater tumor volume reduction compared to administration of engineered T cells expressing the same anti-ROR1 CAR without the knockout (LV) or with expression of a dominant negative form of TGFBRII (DN). The level of expression of CD103, an E-Cadherin binding integrin induced by TGFβ, was assessed, and was observed to be higher in engineered cells expressing the anti-ROR1 CAR and endogenous levels of TGFBRII, compared to cells engineered to express the anti-ROR1 CAR and KO for TGFBR2 or expressing DN-TGFBRII.

As shown in FIGS. 2A and 2B (Donor 1 and 2, respectively), administration of anti-ROR1 CAR-expressing cells that were KO for TGFBR2 or expressing DN-TGFBRII resulted in improved tumor-free survival compared to mice administered T cells engineered only to express the anti-ROR1 CAR, although donor-to-donor variability was observed. Administration of engineered T cells expressing the anti-ROR1 CAR-expressing cells that were KO for TGFBR2 at both the tested low and high doses, resulted in the greatest tumor-free survival in these studies. Administration of the engineered T cells expressing the anti-ROR1 CAR and DN-TGFBRII resulted in improved tumor volume reduction and tumor-free survival compared to administration of the engineered T cells expressing the anti-ROR1 CAR only.

The results were consistent with an observation that inhibition of TGFβ-mediated immune suppression, by either a knockout of the TGFBR2 gene or expression of a dominant negative (DN) TGFBRII, in engineered T cells expressing an exemplary chimeric antigen receptor (CAR), results in improved anti-tumor activity and improved survival of mice administered such cells.

### Example 2 Assessment of Expansion, Tumor Infiltration and Anti-Tumor Activity of CAR-Expressing T Cells with KO or DN TGFBR2

The expansion, tumor infiltration and anti-tumor activity (based on a spheroid assay) of exemplary CAR-expressing cells in which TGFβ signaling is inhibited, by either a knockout of the TGFBR2 gene or expression of a dominant negative (DN) TGFBRII, were assessed.

NSG mice were engrafted with H1975 cells as described in Example 1.B above. On day 24 following tumor engraftment, five (5) mice in each group received a single intravenous (i.v.) injection of 1×10⁶ cells engineered primary human T cells expressing as follows: (1) engineered T cells expressing the anti-ROR1 CAR R12 by lentiviral delivery (LV), (2) engineered T cells expressing the anti-ROR1 CAR R12 by lentiviral delivery and TGFBR2 knockout (KO), or (3) engineered T cells expressing the anti-ROR1 CAR R12 and DN-TGFBRII by lentiviral delivery (DN) at a dose of 1×10⁶ cells, with engineered cells in all groups subject to electroporation.

Tumor volume was monitored up until fourteen (14) days after administration of the engineered cells. At day 14 after administration of the engineered cells, tumor, spleen and blood samples were harvested and assessed by flow cytometry. Isolated tumor-infiltrating lymphocytes (TILs) from the tumor samples were subject to a spheroid killing assay to determine anti-tumor activity.

### A. Tumor Volume

FIGS. 3A (group) and 3B (individual) show the change in tumor volume for the first 14 days after administration of the engineered T cells, prior to collection of the tumor, spleen and blood samples. As shown, administration of anti-ROR1 CAR-expressing cells with a knockout of the TGFBR2 gene (KO) resulted in greater tumor volume reduction compared to administration of engineered T cells expressing the same anti-ROR1 CAR without the knockout (LV) or with expression of a dominant negative form of TGFBRII (DN), consistent with the results described in Example 1.

### B. In Vivo Expansion of CAR-Expressing T Cells and Tumor Infiltration

As shown in FIGS. 4A (blood) and 4B (spleen), the frequency of CAR-expressing CD4+ and CD8+ T cells in the blood or spleen was the highest in mice that had been administered engineered T cells expressing the anti-ROR1 CAR R12 with TGFBR2 KO (KO) compared to the other groups. As shown in FIG. 4C (lower panel), the frequency of CD8+ CAR-expressing cells infiltrating the tumor was higher in mice administered engineered T cells expressing the anti-ROR1 CAR R12 with TGFBR2 KO (KO) compared to the other groups. The mean frequency of CD4+ CAR-expressing cells infiltrating the tumor was similar between mice administered cells expressing anti-ROR1 CAR R12 with TGFBR2 KO (KO) and mice administered cells expressing the same anti-ROR1 CAR with expression of a dominant negative form of TGFBRII (DN) and were higher than the mean frequency in mice administered the anti-ROR1 CAR R12 alone (LV) (FIG. 4C, upper panel). Among tumor-infiltrating engineered cells, the mean percentage of CD103+CD8+ CAR-expressing T cells was lower in engineered cells with TGFBR2 KO compared to the other groups (FIG. 4D, lower panel), while the mean percentage of CD103+CD4+ cells was similar in mice administered anti-ROR1 CAR R12 with TGFBR2 KO (KO) and mice administered the same anti-ROR1 CAR with expression of a dominant negative form of TGFBRII (DN) (FIG. 4D, upper panel).

### C. Anti-Tumor Activity by Spheroid Assay

Anti-tumor activity was assessed in a spheroid killing assay in which isolated tumor-infiltrating lymphocytes (TILs) from the tumor samples from mice administered engineered T cells as described above, were incubated with H1975 tumor spheroids at an effector to target ratio of 1:5 in the presence of a low level of TGFβ in serum-containing media. The H1975 tumor spheroids cells were labeled with a red fluorescent dye to permit monitoring of tumor cell lysis (using the IncuCyte® Live Cell Analysis System, Essen Bioscience), and the incubation was carried out in the presence of a green fluorescent caspase3/7 reagent to monitor apoptosis (using IncuCyte® Caspase-3/7 reagent system). Fluorescence was monitored by microscopy over time for approximately 9 days. T cells recovered from the spleen from mice administered the engineered T cells were also assessed. As controls, H1975 tumor spheroid cells were incubated without the engineered cells (tumor only).

As shown in FIG. 5A, caspase activity was highest in tumor cells recovered from mice administered anti-CAR-expressing T cells with TGFBR2 KO compared to in cells recovered from other treated mice. Likewise, as shown in FIG. 5B, the reduction of spheroid size (as monitored by reduced red fluorescence) was the greatest in tumor cells recovered from mice administered anti-CAR-expressing T cells with TGFBR2 KO (LV KO) compared to in cells recovered from other treated mice. CAR-expressing TGFBR2 KO cells recovered from the spleen also exhibited some caspase activity and anti-tumor activity, at later time points assessed. Engineered T cells expressing the anti-ROR1 CAR and a DN-TGFBRII (LV DN) exhibited some improvements in caspase activity and tumor spheroid lysis compared to cells expressing the anti-ROR1 CAR without modification of TGFBR2. The results were consistent with an observation that CAR-expressing T cells with TGFBR2 knock-out (LV KO) demonstrated improved anti-tumor activity against spheroids as shown by a spheroid killing assay and caspase activity, compared to CAR-expressing cells with a dominant negative (LV DN) TGFBRII or CAR-expressing cells without a knock-out of TGFBR2. The results further support inhibiting TGFβ-mediated immune suppression, for example by KO of TGFBR2 in engineered T cells, to achieve improved activity and function of the engineered cells.

### Example 3 Assessment of Anti-tumor Activity of Fully Human CAR-expressing T Cells with TGFBR2 Knockout

Anti-tumor activity of engineered cells expressing an exemplary fully human chimeric antigen receptor (CAR) was assessed using a spheroid assay.

Primary human CD4+ and CD8+ T cells were isolated, stimulated and engineered to express an exemplary fully human anti-ROR1 CAR, with (fully human KO) a knockout of TGFBR2 or without (fully human WT), generally as described in Example 1.A above, except that the CAR contained a fully human anti-ROR1 scFv antigen-binding domain instead of the scFv derived from a chimeric rabbit/human anti-ROR1. The engineered cells were then cryopreserved. Cells expressing the anti-ROR1 CAR with an scFv antigen-binding domain derived from R12, with a knockout of TGFBR2 (R12 KO) or without (R12 WT), described in Example 1.A above, and cells treated by mock transduction and electroporation without RNPs (mock) or mock transduction with RNPs for TGFBR2 knockout (mock KO) were also assessed as controls.

For the spheroid killing assay, the cryopreserved engineered cells were thawed and incubated with H1975 tumor spheroids at an effector to target ratio of 1:5. Caspase activity (green dye) and spheroid size (red dye) were monitored by microscopy over time for approximately 7 days, generally as described in Example 2.A above. The amount of secreted cytokine interferon-gamma (IFN-γ) was also measured.

As shown in FIGS. 6A (caspase) and 6B (spheroid size), both the fully human anti-ROR1 CAR and the anti-ROR1 CAR R12 with a knockout of TGFBR2 exhibited improved caspase activity and spheroid killing activity compared to cells expressing the same receptor without the knockout of TGFBR2. The result also showed that the production of IFN-γ was generally higher in TGFBR2 KO cells compared to the cells without TGFBR2 KO.

### Example 4 Targeted Knock-In (KI) of Transgene Sequences Encoding a Chimeric Antigen Receptor (CAR) at the Endogenous Transforming Growth Factor Beta Receptor 2 (TGFBR2) Locus in a T Cell

Human T cells were engineered to express an exemplary chimeric antigen receptor (CAR) by targeted integration of the nucleic acid encoding the CAR at the endogenous transforming growth factor beta receptor 2 (TGFBR2) locus, via homology-dependent repair (HDR). The strategy resulted in knock-in of the CAR-encoding sequences at the endogenous TGFBR2 locus and knock-out of the endogenous TGFBR2 locus (KO/KI).

### A. gRNA and Transgene Construct for Targeted KI or Random Integration

Ribonucleoprotein (RNP) complexes were generated for introducing a genetic disruption at the endogenous TGFBR2 locus by CRISPR/Cas9-mediated gene editing. The RNP complexes contained Streptococcus pyogenes Cas9 and a guide RNA (gRNA) with the targeting domain sequence GUGGAUGACCUGGCUAACAG (SEQ ID NO:73), generally as described in Example 1.A above.

Exemplary template polynucleotides were generated for targeted integration (knock-in) of transgene sequences containing nucleic acid sequences encoding an exemplary chimeric antigen receptor (CAR). The transgene sequences included nucleic acid sequences encoding an exemplary CAR specific for the B cell maturation antigen (BCMA), and either a) a human elongation factor 1 alpha (EF1α) promoter with an enhancer (SEQ ID NO:119) to drive the expression of the CAR-encoding sequences under the control of a heterologous promoter (EF1α-CAR); or b) sequences encoding a P2A ribosome skip element (SEQ ID NO:120) upstream of the nucleic acid sequences encoding the exemplary CAR (P2A-CAR), to drive expression of the CAR from the endogenous TGFBR2 promoter upon HDR-mediated targeted integration in-frame into the TGFBR2 open reading frame. The encoded CAR included an scFv that binds to the exemplary target antigen BCMA, an immunoglobulin-derived spacer, a transmembrane domain derived from CD28, a costimulatory region derived from 4-1BB, and a CD3ζ signaling domain.

The general structure of the exemplary template polynucleotides were as follows: [5′ homology arm]-[transgene sequences]-[3′ homology arm]. An exemplary 5′ homology arm contained approximately 600 bp of sequence that is homologous to a portion of the third intron and the fourth exon of the endogenous human TGFBR2 locus (5′ homology arm sequence set forth in SEQ ID NO:69; exon and intron numbering based on isoform 1 as set forth in Table 1 herein), or approximately 600 bp of sequence that is homologous to a portion of the fourth exon (5′ homology arm sequence set forth in SEQ ID NO:71). An exemplary 3′ homology arm contained approximately 600 bp sequence that is homologous to a portion of the fourth intron (3′ homology arm sequence set forth in SEQ ID NO:72).

Integration of the transgene sequences by HDR resulted in a deletion of a portion of the fourth exon, replaced by transgene sequences encoding a CAR and regulatory or multicistronic elements.

As control, CAR-encoding nucleic acid sequences were incorporated into an exemplary HIV-1 derived lentiviral vector for expression of the CAR from sequences introduced into the T cell by random integration. For expression of a dominant negative (DN) form of transforming growth factor beta receptor II (DN-TGFBRII), the lentiviral transduction construct further contained nucleic acid sequences encoding a DN-TGFBRII, generally as described in Example 1.A above

### B. Generation of Engineered T Cells Expressing an Exemplary CAR by Homology Dependent Repair (HDR)

For targeted integration by HDR, adeno-associated virus (AAV) stocks containing vector constructs containing the polynucleotides described above were generated for transduction of cells. For random integration, lentiviral vector particles were produced generally as described in Example 1.A above.

Primary human CD4+ and CD8+ T cells were isolated by immunoaffinity-based selection from human peripheral blood mononuclear cells (PBMCs) obtained from healthy donors. The resulting CD4+ and CD8+ cells (at 1:1 ratio) were stimulated for 72 hours by culturing with an anti-CD3/anti-CD28 reagent. The anti-CD3/anti-CD28 reagent was removed, and the stimulated cells were electroporated with 2.2 μM of the RNP complexes containing TGFBR2-targeting gRNA (containing TGFBR2 targeting domain sequence set forth in SEQ ID NO:73) and Streptococcus pyogenes Cas9 as described above. Within 0 to 3 hours following electroporation, the cells were incubated with AAV stock containing each of the template polynucleotides at 5% volume. Cells electroporated with TGFBR2-targeting RNP but not contacted with AAV preparations (RNP only), mock electroporated and transduced cells (mock), and cells transduced with the lentiviral vector for random integration of CAR-encoding transgene sequences and a dominant negative form of TGFBRII (Lenti DN-TGFBRII) were assessed as controls. The cells were cultured for 3 days and assessed by flow cytometry after staining with an anti-CD4 antibody, an anti-CD8 antibody and a detection agent that specifically binds the CAR, to detect expression of the CAR.

The results are shown in FIG. 7. Introduction of template polynucleotides for targeted integration by HDR at the TGFBR2 locus resulted in expression of the CAR on the surface of the cell in approximately 42-58% of the cells tested (FIG. 7). Expression of the CAR by lentiviral transduction, e.g. as observed in cells engineered to express the CAR and DN-TGFBRII, was higher than the HDR conditions. The results of anti-CD4 and anti-CD8 staining showed that the process for targeted integration of the CAR-encoding sequences did not substantially change the percentage of CD4+ or CD8+ cells in the composition.

The results are consistent with a finding that nucleic acid sequences encoding a CAR can be targeted for integration at the TGFBR2 locus for expression of the CAR under the control of the endogenous TGFBR2 promoter or a heterologous promoter, such as EF1α, generating engineered T cells expressing a CAR.

### Example 5 Anti-Tumor Activity of Engineered T Cells with Targeted Integration of Transgene Sequences Encoding a CAR by Homology Dependent Repair (HDR) at the Endogenous TGFBR2 Locus

The activity of exemplary chimeric antigen receptor (CAR)-expressing cells, engineered by targeted integration at the TGFBR2 locus (KO/KI), or by random integration, with a knockout (KO) of the endogenous TGFBR2 locus or expression of dominant negative TGFBRII (DN), was assessed in a spheroid assay.

### A. Generation of Engineered T Cells by HDR and Expression of CAR

Primary human CD4+ and CD8+ T cells from three (3) human donors were isolated, stimulated and engineered to express an exemplary anti-ROR1 CAR R12 (see Example 1.A) by either: (1) lentiviral delivery alone (LV), (2) lentiviral delivery with TGFBR2 knockout (LV+KO), or (3) lentiviral delivery and expression of dominant negative TGFBRII (LV+DN), each generally as described in Example 1.A above; or by (4) targeted knock-in at the TGFBR2 locus by HDR (KO/KI), substantially as described in Example 4 above except using a nucleic acid encoding the anti-ROR1 CAR R12 and under the control of a different heterologous promoter (MND).

For targeted knock-in, the cells were electroporated with RNP complexes containing TGFBR2-targeting gRNA (containing TGFBR2 targeting domain sequence set forth in SEQ ID NO:73) and Streptococcus pyogenes Cas9 as described above in Example 4.A. Within 0 to 3 hours following electroporation, the cells were incubated with AAV preparations containing template polynucleotides with a structure [5′ homology arm]-[transgene sequences]-[3′ homology arm], with the 5′ homology arm sequence set forth in SEQ ID NO:69 and the 3′ homology arm sequence set forth in SEQ ID NO:72), and the transgene sequences including nucleic acid sequences encoding the anti-ROR1 CAR R12, under operable control of an MND promoter, a synthetic promoter that contains the U3 region of a modified MoMuLV LTR with myeloproliferative sarcoma virus enhancer (sequence set forth in SEQ ID NO:186; see Challita et al. (1995) J. Virol. 69(2):748-755) and linked to an SV40 poly adenylation signal (sequence set forth in SEQ ID NO:185) (KO/KI). The engineered cells were cultured for approximately 7 days after electroporation, and cryopreserved. As controls, cells treated by mock transduction and electroporation without RNPs (mock) or mock transduction with RNPs for TGFBR2 knockout (mock KO) were also assessed. The level of expression of the anti-ROR1 CAR was assessed in each group.

### B. Anti-tumor Activity by Spheroid Assay

For the spheroid killing assay, the engineered cells expressing anti-ROR1 CAR R12 were thawed and incubated with H1975 tumor spheroids at an effector to target ratio of 1:5. Caspase activity (green dye) and spheroid size (red dye) were monitored by microscopy over time for approximately 14 days, generally as described in Example 2.C above.

As shown in FIG. 8A, in this experiment the anti-ROR1 CAR R12 expression (geometric mean fluorescence by flow cytometry) was highest in cells engineered with the exemplary CAR alone by lentiviral delivery (LV) or with DN-TGFBRII (LV+DN) compared to in cells that were delivered the CAR by lentiviral delivery with knock-out of TGFBRII (LV+KO) or by HDR-mediated targeted integration of the CAR at the TGFBR2 locus (KO/KI). Anti-tumor activity, as shown by increased caspase activity (FIG. 8B) and reduced spheroid size (FIG. 8C), was the highest in spheroid cultures incubated with CAR-expressing cells engineered by HDR integration into the TGFBR2 locus (KO/KI). Improved anti-tumor activity also was observed in CAR-expressing cells that were engineered by lentiviral delivery and with KO of the TGFBR2 locus (LV+KO) or with a DN-TGFBRII (LV+DN), compared to cells that were engineered only to express the exemplary CAR (LV).

Similar results for anti-tumor activity were observed in studies using similarly engineered T cells but with a fully human anti-ROR1 CAR.

### C. Spheroid Assay after Prolonged Stimulation

The anti-tumor activity of the engineered cells expressing anti-ROR1 CAR R12 were assessed by a spheroid killing assay after prolonged stimulation. The cryopreserved engineered cells generated as described in Example 5.A, were thawed and subject to a 7-day prolonged stimulation by incubation with beads coated with a recombinant ROR1-Fc fusion protein, which can result in chronic stimulation of the cells and reduced activity. CAR-positive T cells were mixed with ROR1-Fc beads at a ratio of 1 to 1. On day 7, the beads containing ROR1-Fc were removed, and the cells were incubated with H1975 tumor spheroids at an effector to target ratio of 1:5 or 1:10. The percentage of cells expressing the CAR were assessed before and after the prolonged stimulation. Caspase activity (green dye) and spheroid size (red dye) were monitored by microscopy over time for approximately 14 days, generally as described in Example 2.C above. The amount of secreted cytokine interferon-gamma (IFN-γ) was also measured on day 1 of the prolonged stimulation and day 1 of the spheroid killing assay.

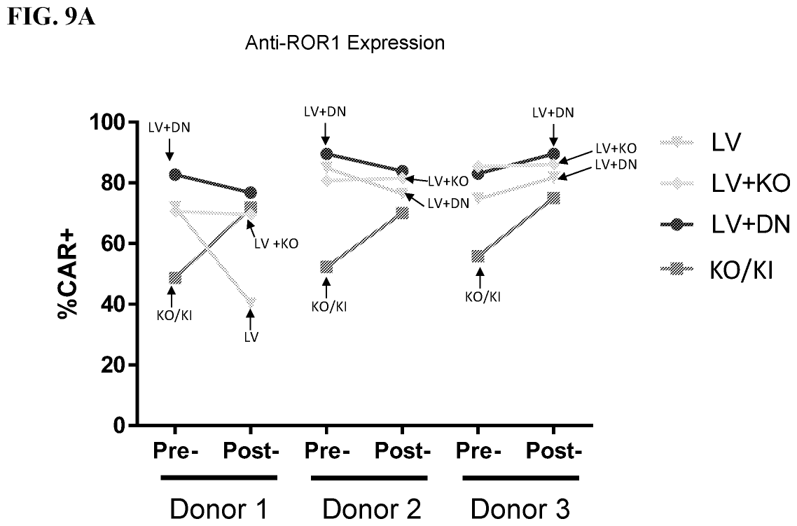

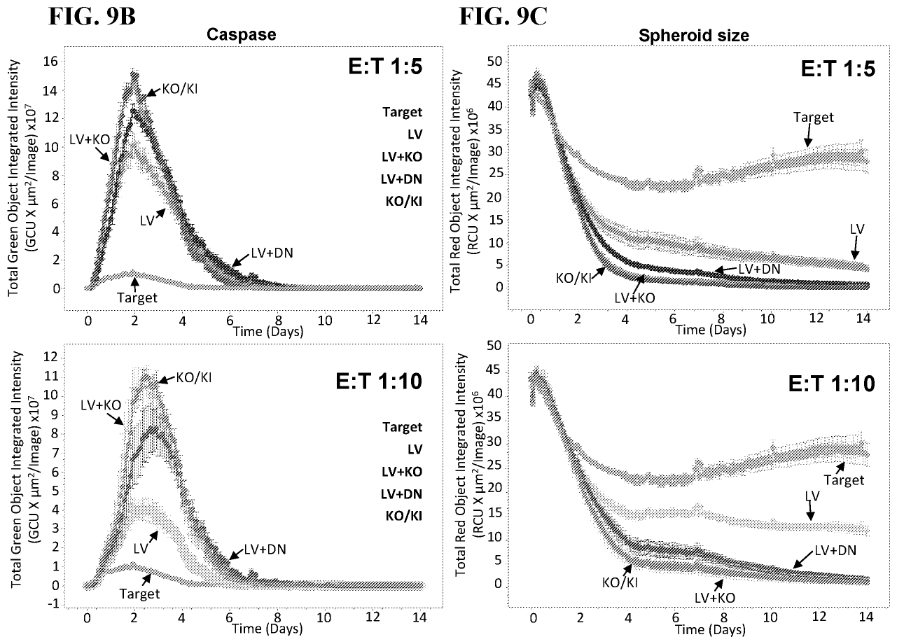

As shown in FIG. 9A, there was an enrichment in the percentage of CAR⁺ cells expressing the anti-ROR1 CAR R12 at thaw prior to the prolonged stimulation (pre) or after the prolonged stimulation (post) in cells engineered with the CAR by HDR-mediated targeted integration of the CAR at the TGFBRII locus (KO/KI). The percentage of CAR-expressing cells at pre- or post-prolonged stimulation were generally similar for the other groups of engineered cells in each of the three donors (with one donor showing a decrease in frequency of expression of the CAR in LV cells engineered to express only the CAR by lentiviral delivery). As shown in FIGS. 9B (caspase) and 9C (spheroid size), cells engineered with the CAR by HDR-mediated targeted integration of the CAR at the TGFBRII locus (KO/KI) or by lentiviral delivery with KO of the TGFBRII locus (LV+KO) exhibited the highest caspase activity and greatest reduction in spheroid size at each of the E:T ratios tested in this study. Improved anti-tumor activity also was observed in CAR-expressing cells that were engineered by lentiviral delivery with a DN-TGFBRII (LV+DN) compared to cells that were engineered only to express the exemplary CAR (LV).

### D. Conclusion

The results are consistent with an observation that targeted knock-in of exemplary CAR-encoding nucleic acid sequences into the endogenous TGFBR2 gene (which also eliminates the expression of the endogenous TGFBR2 gene) results in improved anti-tumor activity, as shown by a spheroid killing assay. The improvement was observed with different exemplary anti-ROR1 CARs, and was similar to or greater than those achieved by cells engineered with CAR-encoding nucleic acids sequences delivered by lentiviral delivery and containing a knock-out of TGFBR2. The results support the use of targeted knock-in of recombinant receptor expressing sequences into the endogenous TGFBR2 gene, for example, by homology-dependent repair (HDR), to produce engineered cells that are less susceptible to, or resistant to, TGFβ-mediated immune suppression and that exhibit improved anti-tumor activity and function.

### Example 6 Generation and Assessment of Anti-Tumor Activity of Engineered T Cells Expressing a Recombinant T Cell Receptor (TCR) with Knock-In (KI) at or Knockout (KO) of TGFBR2

Human T cells were engineered to express an exemplary recombinant T cell receptor (TCR), and a genetic disruption (knockout) of the transforming growth factor beta receptor 2 (TGFBR2) locus, or by targeted integration (knock-in) of the nucleic acid sequences encoding the recombinant TCR at the endogenous TGFBR2 locus.

### A. TGFBR2 KO T Cells Expressing an Exemplary TCR

Primary human CD4+ and CD8+ T cells were isolated, stimulated and engineered to express an exemplary recombinant TCR specific for human papillomavirus 16 (HPV16) E7(11-19) peptide presented on a major histocompatibility complex (MHC) class I molecule, with or without a knockout of TGFBR2. The methods for engineering the cells were generally as described in Example 1.A above, with the exception of using lentiviral vectors that contain nucleic acid sequences encoding the recombinant TCR by either: (1) lentiviral delivery alone (TCR), (2) lentiviral delivery with TGFBR2 knockout (TCR+KO), or (3) lentiviral delivery and mock electroporation without RNPs (TCR EP). As controls, cells treated by mock transduction (mock), mock transduction and electroporation without RNPs (mock EP) or mock transduction and electroporated with RNPs for a TGFBR2 knockout (mock KO) were also assessed.

Anti-tumor activity of the engineered cells expressing an exemplary TCR was assessed by a spheroid killing assay, generally as described in Example 2.C above, with the following exception: anti-HPV16 E7 TCR expressing cells were incubated with tumor spheroids comprising UPCI:SCC152 (ATCC® CRL-3240™) squamous cell carcinoma cells at an E:T ratio of 1:10, with or without 10 ng/mL TGFβ in the media. The amount of secreted cytokines interferon-gamma (IFN-γ), interleukin-2 (IL-2) and tumor necrosis factor alpha (TNF-α) was also measured on day 1 of the spheroid killing assay.

As shown in FIGS. 10A (caspase) and 10B (spheroid size), anti-tumor activity, as shown by increased caspase activity and reduced spheroid size, respectively, was substantially higher for anti-HPV TCR-expressing cells with TGFBR2 KO compared to control cells expressing the same anti-HPV TCR but without a TGFBR2 KO, in studies both with and without addition of TGFβ. The results showed full tumor spheroid clearance even at a sub-optimal E:T ratio of 1:10 by anti-HPV TCR-expressing cells with TGFBR2 KO.

### B. Engineered T Cells Expressing an Exemplary CAR by Homology Dependent Repair (HDR)

Primary human CD4+ and CD8+ T cells from 3 donors (Donors 1, 2 and 3) were isolated, stimulated and engineered to express an exemplary recombinant TCR specific for human papillomavirus 16 (HPV16) by targeted integration via HDR. The methods for engineering the cells were generally as described in Example 4 above, with the following exceptions: the transgene sequences included nucleic acid sequences encoding an exemplary anti-HPV16 TCR, under the control of either a) a human elongation factor 1 alpha (EF1α) promoter (EF1α KO/KI) or b) an MND promoter (MND KO/KI). Cells expressing the recombinant TCR by lentiviral delivery with TGFBR2 knockout (TCR LV TGFBR2 KO) or without TGFBR2 knockout (TCR LV) were also assessed. Additional controls included cells subject to mock treatment (mock) and cells with TGFBR2 knockout that were not engineered to express the recombinant TCR (TGFBR2 KO). The cells were cultured for 8 days and cryopreserved.

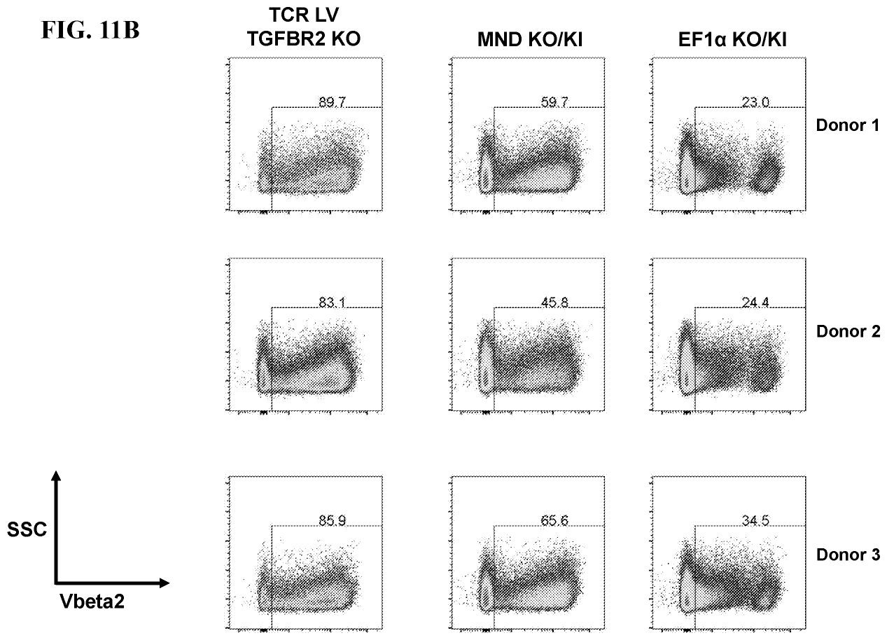

The level of expression of the anti-HPV TCR were assessed in each group by staining with an anti-Vbeta2 antibody that recognizes to the recombinant TCR. The expression of the recombinant TCR in each of the engineered cells is shown in FIGS. 11A and 11B. As shown, the percentage of cells expressing the recombinant TCR was generally higher for cells engineered using lentiviral delivery (TCR LV, see FIG. 11A; or TCR LV TGFBR2 KO, see FIG. 11B), compared to cells engineered by HDR (MND KO/KI or EF1α KO/KI, see FIG. 11B). Approximately 6-9% of the endogenous T cells exhibited non-specific background staining with the anti-Vbeta2 antibody, as shown in the mock group. Among the groups engineered by HDR, expression of the recombinant TCR was higher in cells in which the recombinant TCR was under the control of the MND promoter, compared to under the control of the EF1α promoter.

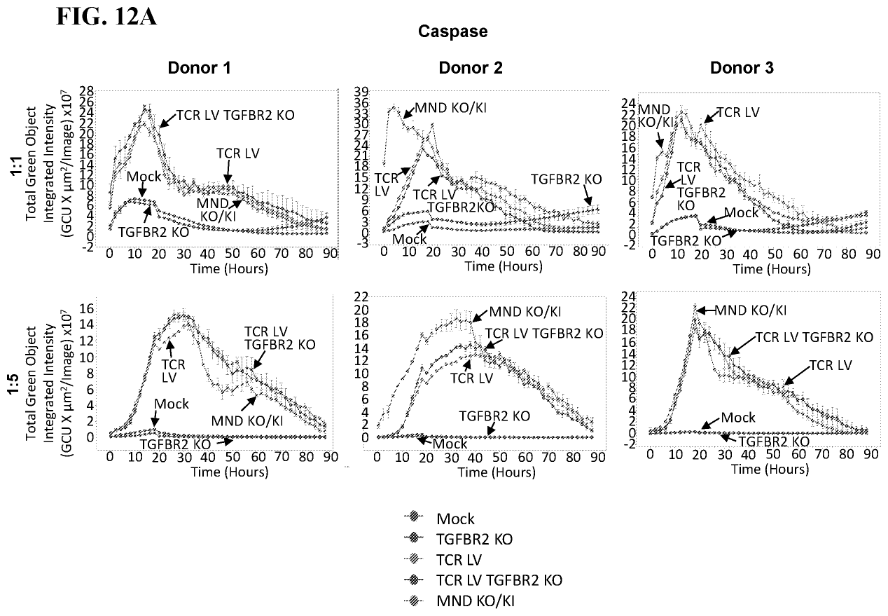

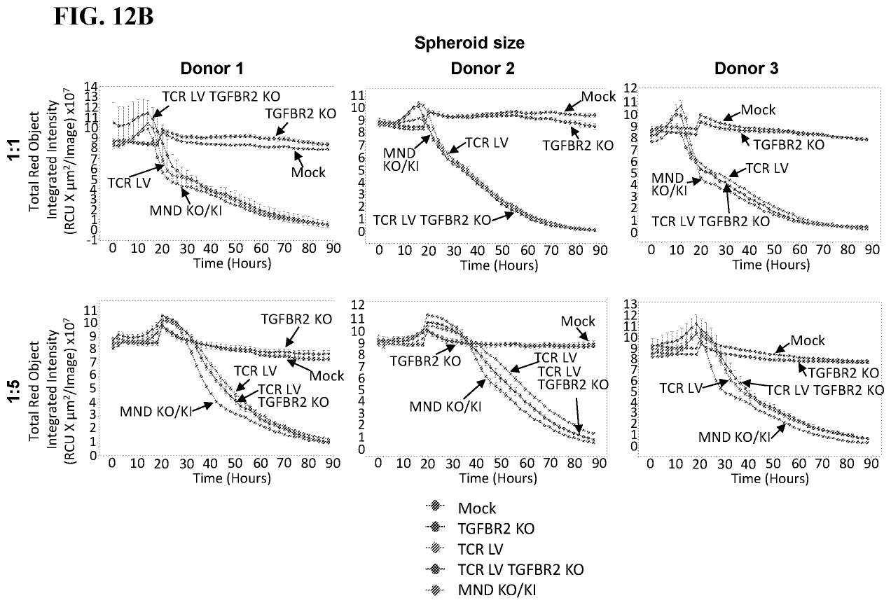

Anti-tumor activity of the engineered cells expressing an exemplary TCR was assessed by a spheroid killing assay, generally as described in Example 6.A above except at an E:T ratio of 1:1 or 1:5 and no addition of exogenous TGFβ. As shown in FIGS. 12A (caspase) and 12B (spheroid size), cells engineered with the recombinant TCR by HDR-mediated targeted integration of the TCR at the TGFBRII locus (MND KO/KI) exhibited the highest caspase activity and greatest reduction in spheroid size at each of the E:T ratios tested in this study. Cells engineered with the recombinant TCR by lentiviral delivery with KO of the TGFBRII locus (TCR LV TGFBR2 KO) also showed similarly high caspase activity.

### C. Conclusion

The results were consistent with an observation that knockout of the endogenous TGFBR2 gene or a targeted knock-in of exemplary TCR-encoding nucleic acid sequences into the endogenous TGFBR2 gene (which also results in knock-out of the endogenous TGFBR2 gene) results in improved anti-tumor activity, as shown by a spheroid killing assay. The results further support the use of targeted knock-in of nucleic acid sequences encoding a recombinant receptor, such as a recombinant TCR, for example, by homology-dependent repair (HDR), to produce engineered cells that are less susceptible to, or resistant to, TGFβ-mediated immune suppression and that exhibit improved anti-tumor activity and function.

### Example 7 Template Polynucleotides for Generation of Dominant Negative TGFBR2 at the Endogenous Locus and Targeted Integration of Transgene Sequences Encoding a CAR

Exemplary template polynucleotides are generated for targeted integration of transgene sequences encoding an exemplary CAR at the endogenous transforming growth factor beta receptor 2 (TGFBR2) locus while also generating a dominant negative TGFBRII (DN-TGFBRII) from the endogenous TGFBR2 locus.

As described in Example 1.A above, DN-TGFBRII lacks the protein kinase domain of the receptor and can interfere with TGF beta (TGFβ) signaling by forming a non-functional receptor complex. An exemplary template polynucleotide with the general structure [5′ homology arm]-[transgene sequences]-[3′ homology arm] is generated. The transgene sequences include i) sequences encoding a CAR, which included an scFv that binds to BCMA, an immunoglobulin-derived spacer, a transmembrane domain derived from CD28 and a costimulatory region derived from 4-1BB, and ii) sequences encoding a P2A ribosome skip element upstream of the nucleic acid sequences encoding the CAR. The 5′ homology arm contains approximately 600 bp of sequence that is homologous to a portion of the third intron and the fourth exon of the endogenous human TGFBR2 locus, including a portion of the sequences encoding the transmembrane domain of TGFBR2 (5′ homology arm sequence set forth in SEQ ID NO:70). The 3′ homology arm contains approximately 600 bp sequence that is homologous to a portion of the fourth intron (3′ homology arm sequence set forth in SEQ ID NO:72).

Integration of the transgene sequences by HDR results in expression of a mRNA transcript encoding DN-TGFBRII-P2A-CAR under the control of the endogenous TGFBR2 promoter, which, upon translation and ribosome skipping, results in DN-TGFBRII polypeptide (fused with cleaved N-terminal portion of the P2A sequence) and a CAR (fused with the cleaved C-terminal proline of the P2A sequence).

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure.

Sequences

| # | SEQUENCE | ANNOTATION |
|---|---|---|
| 1 | ESKYGPPCPPCP | spacer |
| | | (IgG4hinge) |
| | | (aa) |
| 2 | GAATCTAAGTACGGACCGCCCTGCCCCCCTTGCCCT | spacer |
| | | (IgG4hinge) |
| | | (nt) |
| 3 | ESKYGPPCPPCPGQPREPQVYTLPPSQEEMTKNQVSLTCLVKGFYPSDIAVEWESNGQPE | Hinge-C_{H}3 |
| | NNYKTTPPVLDSDGSFFLYSRLTVDKSRWQEGNVFSCSVMHEALHNHYTQKSLSLSLGK | spacer |
| 4 | ESKYGPPCPPCPAPEFLGGPSVFLFPPKPKDTLMISRTPEVTCVVVDVSQEDPEVQFNWY | Hinge-CH2- |
| | VDGVEVHNAKTKPREEQFNSTYRVVSVLTVLHQDWLNGKEYKCKVSNKGLPSSIEKTISK | C_{H}3 spacer |
| | AKGQPREPQVYTLPPSQEEMTKNQVSLTCLVKGFYPSDIAVEWESNGQPENNYKTTPPVL | |
| | DSDGSFFLYSRLTVDKSRWQEGNVFSCSVMHEALHNHYTQKSLSLSLGK | |
| 5 | RWPESPKAQASSVPTAQPQAEGSLAKATTAPATTRNTGRGGEEKKKEKEKEEQEERETKT | IgD-hinge-Fc |
| | PECPSHTQPLGVYLLTPAVQDLWLRDKATFTCFVVGSDLKDAHLTWEVAGKVPTGGVEEG | |
| | LLERHSNGSQSQHSRLTLPRSLWNAGTSVTCTLNHPSLPPQRLMALREPAAQAPVKLSLN | |
| | LLASSDPPEAASWLLCEVSGFSPPNILLMWLEDQREVNTSGFAPARPPPQPGSTTFWAWS | |
| | VLRVPAPPSPQPATYTCVVSHEDSRTLLNASRSLEVSYVTDH | |
| 6 | LEGGGEGRGSLLTCGDVEENPGPR | T2A |
| 7 | MLLLVTSLLLCELPHPAFLLIPRKVCNGIGIGEFKDSLSINATNIKHFKNCTSISGDLHI | tEGFR |
| | LPVAFRGDSFTHTPPLDPQELDILKTVKEITGFLLIQAWPENRTDLHAFENLEIIRGRTK | |
| | QHGQFSLAVVSLNITSLGLRSLKEISDGDVIISGNKNLCYANTINWKKLFGTSGQKTKII | |
| | SNRGENSCKATGQVCHALCSPEGCWGPEPRDCVSCRNVSRGRECVDKCNLLEGEPREFVE | |
| | NSECIQCHPECLPQAMNITCTGRGPDNCIQCAHYIDGPHCVKTCPAGVMGENNTLVWKYA | |
| | DAGHVCHLCHPNCTYGCTGPGLEGCPTNGPKIPSIATGMVGALLLLLVVALGIGLFM | |
| 8 | FWVLVVVGGVLACYSLLVTVAFIIFWV | CD28 (aa 153- |
| | | 179 of Uniprot |
| | | P10747) |
| 9 | IEVMYPPPYLDNEKSNGTIIHVKGKHLCPSPLFPGPSKP | CD28 (aa 114- |
| | FWVLVVVGGVLACYSLLVTVAFIIFWV | 179 of Uniprot |
| | | P10747) |
| 10 | RSKRSRLLHSDYMNMTPRRPGPTRKHYQPYAPPRDFAAYRS | CD28 (aa 180- |
| | | 220 of Uniprot |
| | | P10747) |
| 11 | RSKRSRGGHSDYMNMTPRRPGPTRKHYQPYAPPRDFAAYRS | CD28 (LL to |
| | | GG) |
| 12 | KRGRKKLLYIFKQPFMRPVQTTQEEDGCSCRFPEEEEGGCEL | 4-1BB (aa 214- |
| | | 255 of |
| | | Q07011.1) |
| 13 | RVKFSRSADAPAYQQGQNQLYNELNLGRREEYDVLDKRRGRDPEMGGKPRRKNPQEGLYN | CD3 zeta |
| | ELQKDKMAEAYSEIGMKGERRRGKGHDGLYQGLSTATKDTYDALHMQALPPR | |
| 14 | RVKFSRSAEPPAYQQGQNQLYNELNLGRREEYDVLDKRRGRDPEMGGKPRRKNPQEGLYN | CD3 zeta |
| | ELQKDKMAEAYSEIGMKGERRRGKGHDGLYQGLSTATKDTYDALHMQALPPR | |
| 15 | RVKFSRSADAPAYKQGQNQLYNELNLGRREEYDVLDKRRGRDPEMGGKPRRKNPQEGLYN | CD3 zeta |
| | ELQKDKMAEAYSEIGMKGERRRGKGHDGLYQGLSTATKDTYDALHMQALPPR | |
| 16 | RKVCNGIGIGEFKDSLSINATNIKHFKNCTSISGDLHILPVAFRGDSFTHTPPLDPQELD | tEGFR |
| | ILKTVKEITGFLLIQAWPENRTDLHAFENLEIIRGRTKQHGQFSLAVVSLNITSLGLRSL | |
| | KEISDGDVIISGNKNLCYANTINWKKLFGTSGQKTKIISNRGENSCKATGQVCHALCSPE | |
| | GCWGPEPRDCVSCRNVSRGRECVDKCNLLEGEPREFVENSECIQCHPECLPQAMNITCTG | |
| | RGPDNCIQCAHYIDGPHCVKTCPAGVMGENNTLVWKYADAGHVCHLCHPNCTYGCTGPGL | |
| | EGCPTNGPKIPSIATGMVGALLLLLVVALGIGLFM | |
| 17 | EGRGSLLTCGDVEENPGP | T2A |
| 18 | GSGATNFSLLKQAGDVEENPGP | P2A |
| 19 | ATNFSLLKQAGDVEENPGP | P2A |
| 20 | QCTNYALLKLAGDVESNPGP | E2A |
| 21 | VKQTLNFDLLKLAGDVESNPGP | F2A |
| 22 | -PGGG-(SGGGG)₅-P-wherein P is proline, G is glycine and S | Linker |
| | is serine | |
| 23 | GSADDAKKDAAKKDGKS | Linker |
| 24 | atgcttctcctggtgacaagccttctgctctgtgagttaccacacccagcattcctcctg | GMCSFR alpha |
| | atccca | chain signal |
| | | sequence |
| 25 | MLLLVTSLLLCELPHPAFLLIP | GMCSFR alpha |
| | | chain signal |
| | | sequence |
| 26 | MALPVTALLLPLALLLHA | CD8 alpha |
| | | signal peptide |
| 27 | EPKSCDKTHTCPPCP | Hinge |
| 28 | ERKCCVECPPCP | Hinge |
| 29 | ELKTPLGDTHTCPRCPEPKSCDTPPPCPRCPEPKSCDTPPPCPRCPEPKSCDTPPPCPRC | Hinge |
| | P | |
| 30 | ESKYGPPCPSCP | Hinge |
| 31 | X₁PPX₂P | Hinge |
| | X₁ is glycine, cysteine or arginine | |
| | X₂ is cysteine or threonine | |
| 32 | Tyr Gly Pro Pro Cys Pro Pro Cys Pro | Hinge |
| 33 | Lys Tyr Gly Pro Pro Cys Pro Pro Cys Pro | Hinge |
| 34 | Glu Val Val Val Lys Tyr Gly Pro Pro Cys Pro Pro Cys Pro | Hinge |
| 35 | RASQDISKYLN | CDR L1 |
| 36 | SRLHSGV | CDR L2 |
| 37 | GNTLPYTFG | CDR L3 |
| 38 | DYGVS | CDR H1 |
| 39 | VIWGSETTYYNSALKS | CDR H2 |
| 40 | YAMDYWG | CDR H3 |
| 41 | EVKLQESGPGLVAPSQSLSVTCTVSGVSLPDYGVSWIRQPPRKGLEWLGVIWGSETTYYN | VH |
| | SALKSRLTIIKDNSKSQVFLKMNSLQTDDTAIYYCAKHYYYGGSYAMDYWGQGTSVTVSS | |
| 42 | DIQMTQTTSSLSASLGDRVTISCRASQDISKYLNWYQQKPDGTVKLLIYHTSRLHSGVPS | VL |
| | RFSGSGSGTDYSLTISNLEQEDIATYFCQQGNTLPYTFGGGTKLEIT | |
| 43 | DIQMTQTTSSLSASLGDRVTISCRASQDISKYLNWYQQKPDGTVKLLIYHTSRLHSGVPS | scFv |
| | RFSGSGSGTDYSLTISNLEQEDIATYFCQQGNTLPYTFGGGTKLEITGSTSGSGKPGSGE | |
| | GSTKGEVKLQESGPGLVAPSQSLSVTCTVSGVSLPDYGVSWIRQPPRKGLEWLGVIWGSE | |
| | TTYYNSALKSRLTIIKDNSKSQVFLKMNSLQTDDTAIYYCAKHYYYGGSYAMDYWGQGTS | |
| | VTVSS | |
| 44 | KASQNVGTNVA | CDR L1 |
| 45 | SATYRNS | CDR L2 |
| 46 | QQYNRYPYT | CDR L3 |
| 47 | SYWMN | CDR H1 |
| 48 | QIYPGDGDTNYNGKFKG | CDR H2 |
| 49 | KTISSVVDFYFDY | CDR H3 |
| 50 | EVKLQQSGAELVRPGSSVKISCKASGYAFSSYWMNWVKQRPGQGLEWIGQIYPGDGDTNY | VH |
| | NGKFKGQATLTADKSSSTAYMQLSGLTSEDSAVYFCARKTISSVVDFYFDYWGQGTTVTV | |
| | SS | |
| 51 | DIELTQSPKFMSTSVGDRVSVTCKASQNVGTNVAWYQQKPGQSPKPLIYSATYRNSGVPD | VL |
| | RFTGSGSGTDFTLTITNVQSKDLADYFCQQYNRYPYTSGGGTKLEIKR | |
| 52 | GGGGSGGGGSGGGGS | Linker |
| 53 | EVKLQQSGAELVRPGSSVKISCKASGYAFSSYWMNWVKQRPGQGLEWIGQIYPGDGDTNY | scFv |
| | NGKFKGQATLTADKSSSTAYMQLSGLTSEDSAVYFCARKTISSVVDFYFDYWGQGTTVTV | |
| | SSGGGGSGGGGSGGGGSDIELTQSPKFMSTSVGDRVSVTCKASQNVGTNVAWYQQKPGQS | |
| | PKPLIYSATYRNSGVPDRFTGSGSGTDFTLTITNVQSKDLADYFCQQYNRYPYTSGGGTK | |
| | LEIKR | |
| 54 | HYYYGGSYAMDY | HC-CDR3 |
| 55 | HTSRLHS | LC-CDR2 |
| 56 | QQGNTLPYT | LC-CDR3 |
| 57 | gacatccagatgacccagaccacctccagcctgagcgccagcctgggcgaccgggtgacc | Sequence |
| | atcagctgccgggccagccaggacatcagcaagtacctgaactggtatcagcagaagccc | encoding scFv |
| | gacggcaccgtcaagctgctgatctaccacaccagccggctgcacagcggcgtgcccagc | |
| | cggtttagcggcagcggctccggcaccgactacagcctgaccatctccaacctggaacag | |
| | gaagatatcgccacctacttttgccagcagggcaacacactgccctacacctttggcggc | |
| | ggaacaaagctggaaatcaccggcagcacctccggcagcggcaagcctggcagcggcgag | |
| | ggcagcaccaagggcgaggtgaagctgcaggaaagcggccctggcctggtggcccccagc | |
| | cagagcctgagcgtgacctgcaccgtgagcggcgtgagcctgcccgactacggcgtgagc | |
| | tggatccggcagccccccaggaagggcctggaatggctgggcgtgatctggggcagcgag | |
| | accacctactacaacagcgccctgaagagccggctgaccatcatcaaggacaacagcaag | |
| | agccaggtgttcctgaagatgaacagcctgcagaccgacgacaccgccatctactactgc | |
| | gccaagcactactactacggcggcagctacgccatggactactggggccagggcaccagc | |
| | gtgaccgtgagcagc | |
| 58 | GSTSGSGKPGSGEGSTKG | Linker |
| 59 | MGRGLLRGLWPLHIVLWTRIASTIPPHVQKSVNNDMIVTDNNGAVKFPQLCKFCDVRFST | Human TGF- |
| | CDNQKSCMSNCSITSICEKPQEVCVAVWRKNDENITLETVCHDPKLPYHDFILEDAASPK | beta receptor |
| | CIMKEKKKPGETFFMCSCSSDECNDNIIFSEEYNTSNPDLLLVIFQVTGISLLPPLGVAI | type-2 (TGFR2) |
| | SVIIIFYCYRVNRQQKLSSTWETGKTRKLMEFSEHCAIILEDDRSDISSTCANNINHNTE | isoform 1 |
| | LLPIELDTLVGKGRFAEVYKAKLKQNTSEQFETVAVKIFPYEEYASWKTEKDIFSDINLK | Uniprot |
| | HENILQFLTAEERKTELGKQYWLITAFHAKGNLQEYLTRHVISWEDLRKLGSSLARGIAH | P37173-1 |
| | LHSDHTPCGRPKMPIVHRDLKSSNILVKNDLTCCLCDFGLSLRLDPTLSVDDLANSGQVG | |
| | TARYMAPEVLESRMNLENVESFKQTDVYSMALVLWEMTSRCNAVGEVKDYEPPFGSKVRE | |
| | HPCVESMKDNVLRDRGRPEIPSFWLNHQGIQMVCETLTECWDHDPEARLTAQCVAERFSE | |
| | LEHLDRLSGRSCSEEKIPEDGSLNTTK | |
| 60 | MGRGLLRGLWPLHIVLWTRIASTIPPHVQKSDVEMEAQKDEIICPSCNRTAHPLRHINND | Human TGF- |
| | MIVTDNNGAVKFPQLCKFCDVRFSTCDNQKSCMSNCSITSICEKPQEVCVAVWRKNDENI | beta receptor |
| | TLETVCHDPKLPYHDFILEDAASPKCIMKEKKKPGETFFMCSCSSDECNDNIIFSEEYNT | type-2 (TGFR2) |
| | SNPDLLLVIFQVTGISLLPPLGVAISVIIIFYCYRVNRQQKLSSTWETGKTRKLMEFSEH | isoform 2 |
| | CAIILEDDRSDISSTCANNINHNTELLPIELDTLVGKGRFAEVYKAKLKQNTSEQFETVA | Uniprot |
| | VKIFPYEEYASWKTEKDIFSDINLKHENILQFLTAEERKTELGKQYWLITAFHAKGNLQE | P37173-2 |
| | YLTRHVISWEDLRKLGSSLARGIAHLHSDHTPCGRPKMPIVHRDLKSSNILVKNDLTCCL | |
| | CDFGLSLRLDPTLSVDDLANSGQVGTARYMAPEVLESRMNLENVESFKQTDVYSMALVLW | |
| | EMTSRCNAVGEVKDYEPPFGSKVREHPCVESMKDNVLRDRGRPEIPSFWLNHQGIQMVCE | |
| | TLTECWDHDPEARLTAQCVAERFSELEHLDRLSGRSCSEEKIPEDGSLNTTK | |
| 61 | ggagagggagaaggctctcgggcggagagaggtcctgcccagctgttggcgaggagtttc | Human TGF- |
| | ctgtttcccccgcagcgctgagttgaagttgagtgagtcactcgcgcgcacggagcgacg | beta receptor |
| | acacccccgcgcgtgcacccgctcgggacaggagccggactcctgtgcagcttccctcgg | type-2 (TGFR2) |
| | ccgccgggggcctccccgcgcctcgccggcctccaggccccctcctggctggcgagcggg | transcript |
| | cgccacatctggcccgcacatctgcgctgccggcccggcgcggggtccggagagggcgcg | variant B |
| | gcgcggaggcgcagccaggggtccgggaaggcgccgtccgctgcgctgggggctcggtct | NCBI |
| | atgacgagcagcggggtctgccatgggtcgggggctgctcaggggcctgtggccgctgca | Reference |
| | catcgtcctgtggacgcgtatcgccagcacgatcccaccgcacgttcagaagtcggttaa | Sequence: |
| | taacgacatgatagtcactgacaacaacggtgcagtcaagtttccacaactgtgtaaatt | NM_003242.5 |
| | ttgtgatgtgagattttccacctgtgacaaccagaaatcctgcatgagcaactgcagcat | |
| | cacctccatctgtgagaagccacaggaagtctgtgtggctgtatggagaaagaatgacga | |
| | gaacataacactagagacagtttgccatgaccccaagctcccctaccatgactttattct | |
| | ggaagatgctgcttctccaaagtgcattatgaaggaaaaaaaaaagcctggtgagacttt | |
| | cttcatgtgttcctgtagctctgatgagtgcaatgacaacatcatcttctcagaagaata | |
| | taacaccagcaatcctgacttgttgctagtcatatttcaagtgacaggcatcagcctcct | |
| | gccaccactgggagttgccatatctgtcatcatcatcttctactgctaccgcgttaaccg | |
| | gcagcagaagctgagttcaacctgggaaaccggcaagacgcggaagctcatggagttcag | |
| | cgagcactgtgccatcatcctggaagatgaccgctctgacatcagctccacgtgtgccaa | |
| | caacatcaaccacaacacagagctgctgcccattgagctggacaccctggtggggaaagg | |
| | tcgctttgctgaggtctataaggccaagctgaagcagaacacttcagagcagtttgagac | |
| | agtggcagtcaagatctttccctatgaggagtatgcctcttggaagacagagaaggacat | |
| | cttctcagacatcaatctgaagcatgagaacatactccagttcctgacggctgaggagcg | |
| | gaagacggagttggggaaacaatactggctgatcaccgccttccacgccaagggcaacct | |
| | acaggagtacctgacgcggcatgtcatcagctgggaggacctgcgcaagctgggcagctc | |
| | cctcgcccgggggattgctcacctccacagtgatcacactccatgtgggaggcccaagat | |
| | gcccatcgtgcacagggacctcaagagctccaatatcctcgtgaagaacgacctaacctg | |
| | ctgcctgtgtgactttgggctttccctgcgtctggaccctactctgtctgtggatgacct | |
| | ggctaacagtgggcaggtgggaactgcaagatacatggctccagaagtcctagaatccag | |
| | gatgaatttggagaatgttgagtccttcaagcagaccgatgtctactccatggctctggt | |
| | gctctgggaaatgacatctcgctgtaatgcagtgggagaagtaaaagattatgagcctcc | |
| | atttggttccaaggtgcgggagcacccctgtgtcgaaagcatgaaggacaacgtgttgag | |
| | agatcgagggcgaccagaaattcccagcttctggctcaaccaccagggcatccagatggt | |
| | gtgtgagacgttgactgagtgctgggaccacgacccagaggcccgtctcacagcccagtg | |
| | tgtggcagaacgcttcagtgagctggagcatctggacaggctctcggggaggagctgctc | |
| | ggaggagaagattcctgaagacggctccctaaacactaccaaatagctcttctggggcag | |
| | gctgggccatgtccaaagaggctgcccctctcaccaaagaacagaggcagcaggaagctg | |
| | cccctgaactgatgcttcctggaaaaccaagggggtcactcccctccctgtaagctgtgg | |
| | ggataagcagaaacaacagcagcagggagtgggtgacatagagcattctatgcctttgac | |
| | attgtcataggataagctgtgttagcacttcctcaggaaatgagattgatttttacaata | |
| | gccaataacatttgcactttattaatgcctgtatataaatatgaatagctatgttttata | |
| | tatatatatatatatctatatatgtctatagctctatatatatagccataccttgaaaag | |
| | agacaaggaaaaacatcaaatattcccaggaaattggttttattggagaactccagaacc | |
| | aagcagagaaggaagggacccatgacagcattagcatttgacaatcacacatgcagtggt | |
| | tctctgactgtaaaacagtgaactttgcatgaggaaagaggctccatgtctcacagccag | |
| | ctatgaccacattgcacttgcttttgcaaaataatcattccctgcctagcacttctcttc | |
| | tggccatggaactaagtacagtggcactgtttgaggaccagtgttcccggggttcctgtg | |
| | tgcccttatttctcctggacttttcatttaagctccaagccccaaatctggggggctagt | |
| | ttagaaactctccctcaacctagtttagaaactctaccccatctttaataccttgaatgt | |
| | tttgaaccccactttttaccttcatgggttgcagaaaaatcagaacagatgtccccatcc | |
| | atgcgattgccccaccatctactaatgaaaaattgttctttttttcatctttcccctgca | |
| | cttatgttactattctctgctcccagccttcatccttttctaaaaaggagcaaattctca | |
| | ctctaggctttatcgtgtttactttttcattacacttgacttgattttctagttttctat | |
| | acaaacaccaatgggttccatctttctgggctcctgattgctcaagcacagtttggcctg | |
| | atgaagaggatttcaactacacaatactatcattgtcaggactatgacctcaggcactct | |
| | aaacatatgttttgtttggtcagcacagcgtttcaaaaagtgaagccactttataaatat | |
| | ttggagattttgcaggaaaatctggatccccaggtaaggatagcagatggttttcagtta | |
| | tctccagtccacgttcacaaaatgtgaaggtgtggagacacttacaaagctgcctcactt | |
| | ctcactgtaaacattagctctttccactgcctacctggaccccagtctaggaattaaatc | |
| | tgcacctaaccaaggtcccttgtaagaaatgtccattcaagcagtcattctctgggtata | |
| | taatatgattttgactaccttatctggtgttaagatttgaagttggccttttattggact | |
| | aaaggggaactcctttaagggtctcagttagcccaagtttcttttgcttatatgttaata | |
| | gttttaccctctgcattggagagaggagtgctttactccaagaagctttcctcatggtta | |
| | ccgttctctccatcatgccagccttctcaacctttgcagaaattactagagaggatttga | |
| | atgtgggacacaaaggtcccatttgcagttagaaaatttgtgtccacaaggacaagaaca | |
| | aagtatgagctttaaaactccataggaaacttgttaatcaacaaagaagtgttaatgctg | |
| | caagtaatctcttttttaaaactttttgaagctacttattttcagccaaataggaatatt | |
| | agagagggactggtagtgagaatatcagctctgtttggatggtggaaggtctcattttat | |
| | tgagatttttaagatacatgcaaaggtttggaaatagaacctctaggcaccctcctcagt | |
| | gtgggtgggctgagagttaaagacagtgtggctgcagtagcatagaggcgcctagaaatt | |
| | ccacttgcaccgtagggcatgctgataccatcccaatagctgttgcccattgacctctag | |
| | tggtgagtttctagaatactggtccattcatgagatattcaagattcaagagtattctca | |
| | cttctgggttatcagcataaactggaatgtagtgtcagaggatactgtggcttgttttgt | |
| | ttatgtttttttttcttattcaagaaaaaagaccaaggaataacattctgtagttcctaa | |
| | aaatactgacttttttcactactatacataaagggaaagttttattcttttatggaacac | |
| | ttcagctgtactcatgtattaaaataggaatgtgaatgctatatactctttttatatcaa | |
| | aagtctcaagcacttatttttattctatgcattgtttgtcttttacataaataaaatgtt | |
| | tattagattgaataaagcaaaatactcaggtgagcatcctgcctcctgttcccattccta | |
| | gtagctaaa | |
| 62 | ggagagggagaaggctctcgggcggagagaggtcctgcccagctgttggcgaggagtttc | Human TGF- |
| | ctgtttcccccgcagcgctgagttgaagttgagtgagtcactcgcgcgcacggagcgacg | beta receptor |
| | acacccccgcgcgtgcacccgctcgggacaggagccggactcctgtgcagcttccctcgg | type-2 (TGFR2) |
| | ccgccgggggcctccccgcgcctcgccggcctccaggccccctcctggctggcgagcggg | transcript |
| | cgccacatctggcccgcacatctgcgctgccggcccggcgcggggtccggagagggcgcg | variant A |
| | gcgcggaggcgcagccaggggtccgggaaggcgccgtccgctgcgctgggggctcggtct | NCBI |
| | atgacgagcagcggggtctgccatgggtcgggggctgctcaggggcctgtggccgctgca | Reference |
| | catcgtcctgtggacgcgtatcgccagcacgatcccaccgcacgttcagaagtcggatgt | Sequence: |
| | ggaaatggaggcccagaaagatgaaatcatctgccccagctgtaataggactgcccatcc | NCBI |
| | actgagacatattaataacgacatgatagtcactgacaacaacggtgcagtcaagtttcc | Reference |
| | acaactgtgtaaattttgtgatgtgagattttccacctgtgacaaccagaaatcctgcat | Sequence: |
| | gagcaactgcagcatcacctccatctgtgagaagccacaggaagtctgtgtggctgtatg | NM_001024847.2 |
| | gagaaagaatgacgagaacataacactagagacagtttgccatgaccccaagctccccta | |
| | ccatgactttattctggaagatgctgcttctccaaagtgcattatgaaggaaaaaaaaaa | |
| | gcctggtgagactttcttcatgtgttcctgtagctctgatgagtgcaatgacaacatcat | |
| | cttctcagaagaatataacaccagcaatcctgacttgttgctagtcatatttcaagtgac | |
| | aggcatcagcctcctgccaccactgggagttgccatatctgtcatcatcatcttctactg | |
| | ctaccgcgttaaccggcagcagaagctgagttcaacctgggaaaccggcaagacgcggaa | |
| | gctcatggagttcagcgagcactgtgccatcatcctggaagatgaccgctctgacatcag | |
| | ctccacgtgtgccaacaacatcaaccacaacacagagctgctgcccattgagctggacac | |
| | cctggtggggaaaggtcgctttgctgaggtctataaggccaagctgaagcagaacacttc | |
| | agagcagtttgagacagtggcagtcaagatctttccctatgaggagtatgcctcttggaa | |
| | gacagagaaggacatcttctcagacatcaatctgaagcatgagaacatactccagttcct | |
| | gacggctgaggagcggaagacggagttggggaaacaatactggctgatcaccgccttcca | |
| | cgccaagggcaacctacaggagtacctgacgcggcatgtcatcagctgggaggacctgcg | |
| | caagctgggcagctccctcgcccgggggattgctcacctccacagtgatcacactccatg | |
| | tgggaggcccaagatgcccatcgtgcacagggacctcaagagctccaatatcctcgtgaa | |
| | gaacgacctaacctgctgcctgtgtgactttgggctttccctgcgtctggaccctactct | |
| | gtctgtggatgacctggctaacagtgggcaggtgggaactgcaagatacatggctccaga | |
| | agtcctagaatccaggatgaatttggagaatgttgagtccttcaagcagaccgatgtcta | |
| | ctccatggctctggtgctctgggaaatgacatctcgctgtaatgcagtgggagaagtaaa | |
| | agattatgagcctccatttggttccaaggtgcgggagcacccctgtgtcgaaagcatgaa | |
| | ggacaacgtgttgagagatcgagggcgaccagaaattcccagcttctggctcaaccacca | |
| | gggcatccagatggtgtgtgagacgttgactgagtgctgggaccacgacccagaggcccg | |
| | tctcacagcccagtgtgtggcagaacgcttcagtgagctggagcatctggacaggctctc | |
| | ggggaggagctgctcggaggagaagattcctgaagacggctccctaaacactaccaaata | |
| | gctcttctggggcaggctgggccatgtccaaagaggctgcccctctcaccaaagaacaga | |
| | ggcagcaggaagctgcccctgaactgatgcttcctggaaaaccaagggggtcactcccct | |
| | ccctgtaagctgtggggataagcagaaacaacagcagcagggagtgggtgacatagagca | |
| | ttctatgcctttgacattgtcataggataagctgtgttagcacttcctcaggaaatgaga | |
| | ttgatttttacaatagccaataacatttgcactttattaatgcctgtatataaatatgaa | |
| | tagctatgttttatatatatatatatatatctatatatgtctatagctctatatatatag | |
| | ccataccttgaaaagagacaaggaaaaacatcaaatattcccaggaaattggttttattg | |
| | gagaactccagaaccaagcagagaaggaagggacccatgacagcattagcatttgacaat | |
| | cacacatgcagtggttctctgactgtaaaacagtgaactttgcatgaggaaagaggctcc | |
| | atgtctcacagccagctatgaccacattgcacttgcttttgcaaaataatcattccctgc | |
| | ctagcacttctcttctggccatggaactaagtacagtggcactgtttgaggaccagtgtt | |
| | cccggggttcctgtgtgcccttatttctcctggacttttcatttaagctccaagccccaa | |
| | atctggggggctagtttagaaactctccctcaacctagtttagaaactctaccccatctt | |
| | taataccttgaatgttttgaaccccactttttaccttcatgggttgcagaaaaatcagaa | |
| | cagatgtccccatccatgcgattgccccaccatctactaatgaaaaattgttcttttttt | |
| | catctttcccctgcacttatgttactattctctgctcccagccttcatccttttctaaaa | |
| | aggagcaaattctcactctaggctttatcgtgtttactttttcattacacttgacttgat | |
| | tttctagttttctatacaaacaccaatgggttccatctttctgggctcctgattgctcaa | |
| | gcacagtttggcctgatgaagaggatttcaactacacaatactatcattgtcaggactat | |
| | gacctcaggcactctaaacatatgttttgtttggtcagcacagcgtttcaaaaagtgaag | |
| | ccactttataaatatttggagattttgcaggaaaatctggatccccaggtaaggatagca | |
| | gatggttttcagttatctccagtccacgttcacaaaatgtgaaggtgtggagacacttac | |
| | aaagctgcctcacttctcactgtaaacattagctctttccactgcctacctggaccccag | |
| | tctaggaattaaatctgcacctaaccaaggtcccttgtaagaaatgtccattcaagcagt | |
| | cattctctgggtatataatatgattttgactaccttatctggtgttaagatttgaagttg | |
| | gccttttattggactaaaggggaactcctttaagggtctcagttagcccaagtttctttt | |
| | gcttatatgttaatagttttaccctctgcattggagagaggagtgctttactccaagaag | |
| | ctttcctcatggttaccgttctctccatcatgccagccttctcaacctttgcagaaatta | |
| | ctagagaggatttgaatgtgggacacaaaggtcccatttgcagttagaaaatttgtgtcc | |
| | acaaggacaagaacaaagtatgagctttaaaactccataggaaacttgttaatcaacaaa | |
| | gaagtgttaatgctgcaagtaatctcttttttaaaactttttgaagctacttattttcag | |
| | ccaaataggaatattagagagggactggtagtgagaatatcagctctgtttggatggtgg | |
| | aaggtctcattttattgagatttttaagatacatgcaaaggtttggaaatagaacctcta | |
| | ggcaccctcctcagtgtgggtgggctgagagttaaagacagtgtggctgcagtagcatag | |
| | aggcgcctagaaattccacttgcaccgtagggcatgctgataccatcccaatagctgttg | |
| | cccattgacctctagtggtgagtttctagaatactggtccattcatgagatattcaagat | |
| | tcaagagtattctcacttctgggttatcagcataaactggaatgtagtgtcagaggatac | |
| | tgtggcttgttttgtttatgtttttttttcttattcaagaaaaaagaccaaggaataaca | |
| | ttctgtagttcctaaaaatactgacttttttcactactatacataaagggaaagttttat | |
| | tcttttatggaacacttcagctgtactcatgtattaaaataggaatgtgaatgctatata | |
| | ctctttttatatcaaaagtctcaagcacttatttttattctatgcattgtttgtctttta | |
| | cataaataaaatgtttattagattgaataaagcaaaatactcaggtgagcatcctgcctc | |
| | ctgttcccattcctagtagctaaa | |
| 63 | GCAGACCGAUGUCUACUCCA | TGFBR2 |
| | | targeting |
| | | domain |
| | | sequence 1 |
| 64 | CCCCUACCAUGACUUUAUUC | TGFBR2 |
| | | targeting |
| | | domain |
| | | sequence 2 |
| 65 | GACAUCUCGCUGUAAUGCAG | TGFBR2 |
| | | targeting |
| | | domain |
| | | sequence 3 |
| 66 | CACAUGAAGAAAGUCUCACC | TGFBR2 |
| | | targeting |
| | | domain |
| | | sequence 4 |
| 67 | AUGAUAGUCACUGACAACAA | TGFBR2 |
| | | targeting |
| | | domain |
| | | sequence 5 |
| 68 | CUCCAUCUGUGAGAAGCCAC | TGFBR2 |
| | | targeting |
| | | domain |
| | | sequence 6 |
| 69 | TACATGCAGATTTTTTGAAGGCAGAAGCTGTGTCATTTTTTTTCATGTTCCCAATGTCCT | TGFBR2 5′ |
| | GAGCTTAGATAACACTCAGTAAATGGTTTGTCTTTTTATTTGGCAATATTGAGGACCTGC | homology arm |
| | TGTGTGCTAAGTGCAGTTTACAGTAGTGAAGAAGACATGGTACCTTCCAGCATGGAGTTC | sequence 1 |
| | CCTGTCCGTGGGGGATGGCAAGAGTAGGGAAAGACAGATGTGAAATCAAGAGGTAGAGTC | |
| | ATAGTTCATTTAGTTTAAGTTGTACTGAATTGTTACCTAGGAAAAGTATAAGGTGCTATG | |
| | AAAATGTATAAAATAAGACAGTTTTCCAAGTTTTTCTAGGCCTCTCTTAAGCAGTGACAT | |
| | TTAAGCTGAAGTTTGAAGGAAGAGCAGGGGATGACGAACAGATGGCCAGAGGCAGGGAAG | |
| | GCTGAACGAGCATGCACTTGCATCCCTGAAATAAAAATTAACAATATCGTATCTACAAAA | |
| | ACTATGCAGATGCTAAAATCTATAGATGCTCAGGCATGAACCCACTTCCTGACAGTACTT | |
| | ACCTACCACATCCAACTCCTTCTCTCCTTGTTTTGTTTCCCCATCAGAATATAACACCAG | |
| | CAATCCTGAC | |
| 70 | GTGCAGTTTACAGTAGTGAAGAAGACATGGTACCTTCCAGCATGGAGTTCCCTGTCCGTG | TGFBR2 5′ |
| | GGGGATGGCAAGAGTAGGGAAAGACAGATGTGAAATCAAGAGGTAGAGTCATAGTTCATT | homology arm |
| | TAGTTTAAGTTGTACTGAATTGTTACCTAGGAAAAGTATAAGGTGCTATGAAAATGTATA | sequence 2 |
| | AAATAAGACAGTTTTCCAAGTTTTTCTAGGCCTCTCTTAAGCAGTGACATTTAAGCTGAA | |
| | GTTTGAAGGAAGAGCAGGGGATGACGAACAGATGGCCAGAGGCAGGGAAGGCTGAACGAG | |
| | CATGCACTTGCATCCCTGAAATAAAAATTAACAATATCGTATCTACAAAAACTATGCAGA | |
| | TGCTAAAATCTATAGATGCTCAGGCATGAACCCACTTCCTGACAGTACTTACCTACCACA | |
| | TCCAACTCCTTCTCTCCTTGTTTTGTTTCCCCATCAGAATATAACACCAGCAATCCTGAC | |
| | TTGTTGCTAGTCATATTTCAAGTGACAGGCATCAGCCTCCTGCCACCACTGGGAGTTGCC | |
| | ATATCTGTCATCATCATCTTCTACTGCTACCGCGTTAACCGGCAGCAGAAGCTGAGTTCA | |
| 71 | ATGGAGTTCAGCGAGCACTGTGCCATCATCCTGGAAGATGACCGCTCTGACATCAGCTCC | TGFBR2 5′ |
| | ACGTGTGCCAACAACATCAACCACAACACAGAGCTGCTGCCCATTGAGCTGGACACCCTG | homology arm |
| | GTGGGGAAAGGTCGCTTTGCTGAGGTCTATAAGGCCAAGCTGAAGCAGAACACTTCAGAG | sequence 3 |
| | CAGTTTGAGACAGTGGCAGTCAAGATCTTTCCCTATGAGGAGTATGCCTCTTGGAAGACA | |
| | GAGAAGGACATCTTCTCAGACATCAATCTGAAGCATGAGAACATACTCCAGTTCCTGACG | |
| | GCTGAGGAGCGGAAGACGGAGTTGGGGAAACAATACTGGCTGATCACCGCCTTCCACGCC | |
| | AAGGGCAACCTACAGGAGTACCTGACGCGGCATGTCATCAGCTGGGAGGACCTGCGCAAG | |
| | CTGGGCAGCTCCCTCGCCCGGGGGATTGCTCACCTCCACAGTGATCACACTCCATGTGGG | |
| | AGGCCCAAGATGCCCATCGTGCACAGGGACCTCAAGAGCTCCAATATCCTCGTGAAGAAC | |
| | GACCTAACCTGCTGCCTGTGTGACTTTGGGCTTTCCCTGCGTCTGGACCCTACTCTGTCT | |
| 72 | GTAAGTTAGAGCTAGTGCTAGATCCCCTTTACCTTGAGCCTGGCCTCACCCTACCTCTTG | TGFBR2 3′ |
| | ATCCATATCTCCTGGCTCTTATCTCAAACAGCCCTGTACTCTGGACACTGGTCTAGGGAA | homology arm |
| | TCTAGCCAAAGTATGGAGTCTGCCTTGAGCATACTCTGCTCTGTCCTGCCTGAGCATTTT | sequence 1 |
| | TGCTAATGGACAGCATTTCTCCTCCTATCTTCAAATCCTTCCCAGTTCAGCACATTTTTT | |
| | CCTCCTGGATCAATCCTCATTTCTCTTCCAGCAAATGTTTTTTCTTTGTTTCAAGCACTG | |
| | TTAGTACTTTACCTCTATTTTTTCCCTCTCTTATGGTTGTACTCAGTCCTTTCTGCTCTA | |
| | TACTAGCTGTAGTTGTGTTGGTTTCTTTGTATTAAAAGCATCGTGGAAGGCAATCTCCCT | |
| | GAAGTCCAAATCTACATCCACATGGTCACCCAAGATATGTAGCACAATGCCTTGAACATT | |
| | GAAAGTAAAATAAGTACTTGTCGACTGAGTGAGCACTTCCACTCTTGAAGCACTCTCACA | |
| | GATTAAAATGGAAATGTTTTTGGCTAAGAAACTATTGGAAGGTGATTGGAAATCACCACA | |
| 73 | GUGGAUGACCUGGCUAACAG | TGFBR2 |
| | | targeting |
| | | domain |
| | | sequence |
| 74 | GCAGACCGATGTCTACTCCA | TGFBR2 target |
| | | sequence 1 |
| 75 | GACATCTCGCTGTAATGCAG | TGFBR2 target |
| | | sequence 2 |
| 76 | ACAGTGATCACACTCCATGT | TGFBR2 target |
| | | sequence 3 |
| 77 | cgtgaggctccggtgcccgtcagtgggcagagcgcacatcgcccacagtccccgagaagt | EF1alpha |
| | tggggggaggggtcggcaattgaaccggtgcctagagaaggtggcgcggggtaaactggg | promoter |
| | aaagtgatgtcgtgtactggctccgcctttttcccgagggtgggggagaaccgtatataa | (GenBank: |
| | gtgcagtagtcgccgtgaacgttctttttcgcaacgggtttgccgccagaacacaggtaa | J04617.1) |
| | gtgccgtgtgtggttcccgcgggcctggcctctttacgggttatggcccttgcgtgcctt | |
| | gaattacttccacgcccctggctgcagtacgtgattcttgatcccgagcttcgggttgga | |
| | agtgggtgggagagttcgaggccttgcgcttaaggagccccttcgcctcgtgcttgagtt | |
| | gaggcctggcctgggcgctggggccgccgcgtgcgaatctggtggcaccttcgcgcctgt | |
| | ctcgctgctttcgataagtctctagccatttaaaatttttgatgacctgctgcgacgctt | |
| | tttttctggcaagatagtcttgtaaatgcgggccaagatctgcacactggtatttcggtt | |
| | tttggggccgcgggcggcgacggggcccgtgcgtcccagcgcacatgttcggcgaggcgg | |
| | ggcctgcgagcgcggccaccgagaatcggacgggggtagtctcaagctggccggcctgct | |
| | ctggtgcctggcctcgcgccgccgtgtatcgccccgccctgggcggcaaggctggcccgg | |
| | tcggcaccagttgcgtgagcggaaagatggccgcttcccggccctgctgcagggagctca | |
| | aaatggaggacgcggcgctcgggagagcgggcgggtgagtcacccacacaaaggaaaagg | |
| | gcctttccgtcctcagccgtcgcttcatgtgactccacggagtaccgggcgccgtccagg | |
| | cacctcgattagttctcgagcttttggagtacgtcgtctttaggttggggggaggggttt | |
| | tatgcgatggagtttccccacactgagtgggtggagactgaagttaggccagcttggcac | |
| | ttgatgtaattctccttggaatttgccctttttgagtttggatcttggttcattctcaag | |
| | cctcagacagtggttcaaagtttttttcttccatttcaggtgtcgtgaa | |
| 78 | CTGACCTCTTCTCTTCCTCCCACAG | human HBB |
| | | splice acceptor |
| | | site |
| 79 | TTTCTCTCCACAG | Human IgG |
| | | splice acceptor |
| | | site |
| 80 | GTAGCTCTGATGAGTGCAAT | TGFBR2 target |
| | | sequence A |
| 81 | ATGAATCTCTTCACTCTAGG | TGFBR2 target |
| | | sequence B |
| 82 | GVQVETISPGDGRTFPKRGQTCVVHYTGMLEDGKKMDSSRDRNKPFKFMLGKQEVIRGWE | FKBP |
| | EGVAQMSVGQRAKLTISPDYAYGATGHPGIIPPHATLVFDVELLKLE | |
| 83 | GVQVETISPGDGRTFPKRGQTCVVHYTGMLEDGKKVDSSRDRNKPFKFMLGKQEVIRGWE | FKBP12v36 |
| | EGVAQMSVGQRAKLTISPDYAYGATGHPGIIPPHATLVFDVELLKLE | |
| 84 | MGSNKSKPKDASQRRR | human C-Src |
| | | acylation motif |
| 85 | MGCXC | dual acylation |
| | | motif |
| 86 | CAAX | CAAX motif |
| 87 | ACAGGAGTACCTGACGCGGC | TGFBR2 target |
| | | sequence C |
| 88 | CTGTTAGCCAGGTCATCCAC | TGFBR2 target |
| | | sequence D |
| 89 | GGGTGTCCAGCTCAATGGGC | TGFBR2 target |
| | | sequence E |
| 90 | TCATAATGCACTTTGGAGAA | TGFBR2 target |
| | | sequence F |
| 91 | TGACTTTATTCTGGAAGATG | TGFBR2 target |
| | | sequence G |
| 92 | GGCCGCTGCACATCGTCCTG | TGFBR2 target |
| | | sequence 4 |
| 93 | GCGGGGTCTGCCATGGGTCG | TGFBR2 target |
| | | sequence 5 |
| 94 | AGTTGCTCATGCAGGATTTC | TGFBR2 target |
| | | sequence 6 |
| 95 | AAGTCATGGTAGGGGAGCTT | TGFBR2 target |
| | | sequence 7 |
| 96 | AGTCATGGTAGGGGAGCTTG | TGFBR2 target |
| | | sequence 8 |
| 97 | NNNNNNNNNNNNNNNNNNNNGUUUUAGAGCUAGAAAUAGCAAGUUAAAAUAAGGCUAGUC | exemplary |
| | CGUUAUCAACUUGAAAAAGUGGCACCGAGUCGGUGC | gRNA |
| | | complementary |
| | | domain |
| 98 | NNNNNNNNNNNNNNNNNNNNGUUUUAGAGCUAUGCUGAAAAGCAUAGCAAGUUAAAAUAA | exemplary |
| | GGCUAGUCCGUUAUCAACUUGAAAAAGUGGCACCGAGUCGGUGC | gRNA |
| | | complementary |
| | | domain |
| 99 | NNNNNNNNNNNNNNNNNNNNGUUUUAGAGCUAUGCUGGAAACAGCAUAGCAAGUUAAAAU | exemplary |
| | AAGGCUAGUCCGUUAUCAACUUGAAAAAGUGGCACCGAGUCGGUGC | gRNA |
| | | complementary |
| | | domain |
| 100 | NNNNNNNNNNNNNNNNNNNNGUUUUAGAGCUAUGCUGUUUUGGAAACAAAACAGCAUAGC | exemplary |
| | AAGUUAAAAUAAGGCUAGUCCGUUAUCAACUUGAAAAAGUGGCACCGAGUCGGUGC | gRNA |
| | | complementary |
| | | domain |
| 101 | NNNNNNNNNNNNNNNNNNNNGUAUUAGAGCUAGAAAUAGCAAGUUAAUAUAAGGCUAGUC | exemplary |
| | CGUUAUCAACUUGAAAAAGUGGCACCGAGUCGGUGC | gRNA |
| 102 | NNNNNNNNNNNNNNNNNNNNGUUUAAGAGCUAGAAAUAGCAAGUUUAAAUAAGGCUAGUC | exemplary |
| | CGUUAUCAACUUGAAAAAGUGGCACCGAGUCGGUGC | gRNA |
| 103 | NNNNNNNNNNNNNNNNNNNNGUAUUAGAGCUAUGCUGUAUUGGAAACAAUACAGCAUAGC | exemplary |
| | AAGUUAAUAUAAGGCUAGUCCGUUAUCAACUUGAAAAAGUGGCACCGAGUCGGUGC | gRNA |
| 104 | AAGGCUAGUCCGUUAUCAACUUGAAAAAGUGGCACCGAGUCGGUGCU | exemplary |
| | | proximal and |
| | | tail domain |
| 105 | AAGGCUAGUCCGUUAUCAACUUGAAAAAGUGGCACCGAGUCGGUGGUGC | exemplary |
| | | proximal and |
| | | tail domain |
| 106 | AAGGCUAGUCCGUUAUCAACUUGAAAAAGUGGCACCGAGUCGGUGCGGAUC | exemplary |
| | | proximal and |
| | | tail domain |
| 107 | AAGGCUAGUCCGUUAUCAACUUGAAAAAGUG | exemplary |
| | | proximal and |
| | | tail domain |
| 108 | AAGGCUAGUCCGUUAUCA | exemplary |
| | | proximal and |
| | | tail domain |
| 109 | AAGGCUAGUCCG | exemplary |
| | | proximal and |
| | | tail domain |
| 110 | NNNNNNNNNNNNNNNNNNNNGUUUUAGAGCUAGAAAUAGCAAGUUAAAAUAAGGCUAGUC | exemplary |
| | CGUUAUCAACUUGAAAAAGUGGCACCGAGUCGGUGCUUUUUU | chimeric gRNA |
| 111 | NNNNNNNNNNNNNNNNNNNNGUUUUAGUACUCUGGAAACAGAAUCUACUAAAACAAGGCA | exemplary |
| | AAAUGCCGUGUUUAUCUCGUCAACUUGUUGGCGAGAUUUUUU | chimeric gRNA |
| 112 | KKPYSIGLDIGTNSVGWAVVTDDYKVPAKKMKVLGNTDKSHIEKNLLGALLFDSGNTAED | Streptococcus |
| | RRLKRTARRRYTRRRNRILYLQEIFSEEMGKVDDSFFHRLEDSFLVTEDKRGERHPIFGN | mutans Cas9 |
| | LEEEVKYHENFPTIYHLRQYLADNPEKVDLRLVYLALAHIIKFRGHFLIEGKFDTRNNDV | |
| | QRLFQEFLAVYDNTFENSSLQEQNVQVEEILTDKISKSAKKDRVLKLFPNEKSNGRFAEF | |
| | LKLIVGNQADFKKHFELEEKAPLQFSKDTYEEELEVLLAQIGDNYAELFLSAKKLYDSIL | |
| | LSGILTVTDVGTKAPLSASMIQRYNEHQMDLAQLKQFIRQKLSDKYNEVFSDVSKDGYAG | |
| | YIDGKTNQEAFYKYLKGLLNKIEGSGYFLDKIEREDFLRKQRTFDNGSIPHQIHLQEMRA | |
| | IIRRQAEFYPFLADNQDRIEKLLTFRIPYYVGPLARGKSDFAWLSRKSADKITPWNFDEI | |
| | VDKESSAEAFINRMTNYDLYLPNQKVLPKHSLLYEKFTVYNELTKVKYKTEQGKTAFFDA | |
| | NMKQEIFDGVFKVYRKVTKDKLMDFLEKEFDEFRIVDLTGLDKENKVFNASYGTYHDLCK | |
| | ILDKDFLDNSKNEKILEDIVLTLTLFEDREMIRKRLENYSDLLTKEQVKKLERRHYTGWG | |
| | RLSAELIHGIRNKESRKTILDYLIDDGNSNRNFMQLINDDALSFKEEIAKAQVIGETDNL | |
| | NQVVSDIAGSPAIKKGILQSLKIVDELVKIMGHQPENIVVEMARENQFTNQGRRNSQQRL | |
| | KGLTDSIKEFGSQILKEHPVENSQLQNDRLFLYYLQNGRDMYTGEELDIDYLSQYDIDHI | |
| | IPQAFIKDNSIDNRVLTSSKENRGKSDDVPSKDVVRKMKSYWSKLLSAKLITQRKFDNLT | |
| | KAERGGLTDDDKAGFIKRQLVETRQITKHVARILDERFNTETDENNKKIRQVKIVTLKSN | |
| | LVSNFRKEFELYKVREINDYHHAHDAYLNAVIGKALLGVYPQLEPEFVYGDYPHFHGHKE | |
| | NKATAKKFFYSNIMNFFKKDDVRTDKNGEIIWKKDEHISNIKKVLSYPQVNIVKKVEEQT | |
| | GGFSKESILPKGNSDKLIPRKTKKFYWDTKKYGGFDSPIVAYSILVIADIEKGKSKKLKT | |
| | VKALVGVTIMEKMTFERDPVAFLERKGYRNVQEENIIKLPKYSLFKLENGRKRLLASARE | |
| | LQKGNEIVLPNHLGTLLYHAKNIHKVDEPKHLDYVDKHKDEFKELLDVVSNFSKKYTLAE | |
| | GNLEKIKELYAQNNGEDLKELASSFINLLTFTAIGAPATFKFFDKNIDRKRYTSTTEILN | |
| | ATLIHQSITGLYETRIDLNKLGGD | |
| 113 | DKKYSIGLDIGTNSVGWAVITDEYKVPSKKFKVLGNTDRHSIKKNLIGALLFDSGETAEA | Streptococcus |
| | TRLKRTARRRYTRRKNRICYLQEIFSNEMAKVDDSFFHRLEESFLVEEDKKHERHPIFGN | pyogenes Cas9 |
| | IVDEVAYHEKYPTIYHLRKKLVDSTDKADLRLIYLALAHMIKFRGHFLIEGDLNPDNSDV | |
| | DKLFIQLVQTYNQLFEENPINASGVDAKAILSARLSKSRRLENLIAQLPGEKKNGLFGNL | |
| | IALSLGLTPNFKSNFDLAEDAKLQLSKDTYDDDLDNLLAQIGDQYADLFLAAKNLSDAIL | |
| | LSDILRVNTEITKAPLSASMIKRYDEHHQDLTLLKALVRQQLPEKYKEIFFDQSKNGYAG | |
| | YIDGGASQEEFYKFIKPILEKMDGTEELLVKLNREDLLRKQRTFDNGSIPHQIHLGELHA | |
| | ILRRQEDFYPFLKDNREKIEKILTFRIPYYVGPLARGNSRFAWMTRKSEETITPWNFEEV | |
| | VDKGASAQSFIERMTNFDKNLPNEKVLPKHSLLYEYFTVYNELTKVKYVTEGMRKPAFLS | |
| | GEQKKAIVDLLFKTNRKVTVKQLKEDYFKKIECFDSVEISGVEDRFNASLGTYHDLLKII | |
| | KDKDFLDNEENEDILEDIVLTLTLFEDREMIEERLKTYAHLFDDKVMKQLKRRRYTGWGR | |
| | LSRKLINGIRDKQSGKTILDFLKSDGFANRNFMQLIHDDSLTFKEDIQKAQVSGQGDSLH | |
| | EHIANLAGSPAIKKGILQTVKVVDELVKVMGRHKPENIVIEMARENQTTQKGQKNSRERM | |
| | KRIEEGIKELGSQILKEHPVENTQLQNEKLYLYYLQNGRDMYVDQELDINRLSDYDVDHI | |
| | VPQSFLKDDSIDNKVLTRSDKNRGKSDNVPSEEVVKKMKNYWRQLLNAKLITQRKFDNLT | |
| | KAERGGLSELDKAGFIKRQLVETRQITKHVAQILDSRMNTKYDENDKLIREVKVITLKSK | |
| | LVSDFRKDFQFYKVREINNYHHAHDAYLNAVVGTALIKKYPKLESEFVYGDYKVYDVRKM | |
| | IAKSEQEIGKATAKYFFYSNIMNFFKTEITLANGEIRKRPLIETNGETGEIVWDKGRDFA | |
| | TVRKVLSMPQVNIVKKTEVQTGGFSKESILPKRNSDKLIARKKDWDPKKYGGFDSPTVAY | |
| | SVLVVAKVEKGKSKKLKSVKELLGITIMERSSFEKNPIDFLEAKGYKEVKKDLIIKLPKY | |
| | SLFELENGRKRMLASAGELQKGNELALPSKYVNFLYLASHYEKLKGSPEDNEQKQLFVEQ | |
| | HKHYLDEIIEQISEFSKRVILADANLDKVLSAYNKHRDKPIREQAENIIHLFTLTNLGAP | |
| | AAFKYFDTTIDRKRYTSTKEVLDATLIHQSITGLYETRIDLSQLGGD | |
| 114 | TKPYSIGLDIGTNSVGWAVTTDNYKVPSKKMKVLGNTSKKYIKKNLLGVLLFDSGITAEG | Streptococcus |
| | RRLKRTARRRYTRRRNRILYLQEIFSTEMATLDDAFFQRLDDSFLVPDDKRDSKYPIFGN | thermophilus |
| | LVEEKAYHDEFPTIYHLRKYLADSTKKADLRLVYLALAHMIKYRGHFLIEGEFNSKNNDI | Cas9 |
| | QKNFQDFLDTYNAIFESDLSLENSKQLEEIVKDKISKLEKKDRILKLFPGEKNSGIFSEF | |
| | LKLIVGNQADFRKCFNLDEKASLHFSKESYDEDLETLLGYIGDDYSDVFLKAKKLYDAIL | |
| | LSGFLTVTDNETEAPLSSAMIKRYNEHKEDLALLKEYIRNISLKTYNEVFKDDTKNGYAG | |
| | YIDGKTNQEDFYVYLKKLLAEFEGADYFLEKIDREDFLRKQRTFDNGSIPYQIHLQEMRA | |
| | ILDKQAKFYPFLAKNKERIEKILTFRIPYYVGPLARGNSDFAWSIRKRNEKITPWNFEDV | |
| | IDKESSAEAFINRMTSFDLYLPEEKVLPKHSLLYETFNVYNELTKVRFIAESMRDYQFLD | |
| | SKQKKDIVRLYFKDKRKVTDKDIIEYLHAIYGYDGIELKGIEKQFNSSLSTYHDLLNIIN | |
| | DKEFLDDSSNEAIIEEIIHTLTIFEDREMIKQRLSKFENIFDKSVLKKLSRRHYTGWGKL | |
| | SAKLINGIRDEKSGNTILDYLIDDGISNRNFMQLIHDDALSFKKKIQKAQIIGDEDKGNI | |
| | KEVVKSLPGSPAIKKGILQSIKIVDELVKVMGGRKPESIVVEMARENQYTNQGKSNSQQR | |
| | LKRLEKSLKELGSKILKENIPAKLSKIDNNALQNDRLYLYYLQNGKDMYTGDDLDIDRLS | |
| | NYDIDHIIPQAFLKDNSIDNKVLVSSASNRGKSDDVPSLEVVKKRKTFWYQLLKSKLISQ | |
| | RKFDNLTKAERGGLSPEDKAGFIQRQLVETRQITKHVARLLDEKFNNKKDENNRAVRTVK | |
| | IITLKSTLVSQFRKDFELYKVREINDFHHAHDAYLNAVVASALLKKYPKLEPEFVYGDYP | |
| | KYNSFRERKSATEKVYFYSNIMNIFKKSISLADGRVIERPLIEVNEETGESVWNKESDLA | |
| | TVRRVLSYPQVNVVKKVEEQNHGLDRGKPKGLFNANLSSKPKPNSNENLVGAKEYLDPKK | |
| | YGGYAGISNSFTVLVKGTIEKGAKKKITNVLEFQGISILDRINYRKDKLNFLLEKGYKDI | |
| | ELIIELPKYSLFELSDGSRRMLASILSTNNKRGEIHKGNQIFLSQKFVKLLYHAKRISNT | |
| | INENHRKYVENHKKEFEELFYYILEFNENYVGAKKNGKLLNSAFQSWQNHSIDELCSSFI | |
| | GPTGSERKGLFELTSRGSAADFEFLGVKIPRYRDYTPSSLLKDATLIHQSVTGLYETRID | |
| | LAKLGEG | |
| 115 | KKPYTIGLDIGTNSVGWAVLTDQYDLVKRKMKIAGDSEKKQIKKNFWGVRLFDEGQTAAD | Listeria innocua |
| | RRMARTARRRIERRRNRISYLQGIFAEEMSKTDANFFCRLSDSFYVDNEKRNSRHPFFAT | Cas9 |
| | IEEEVEYHKNYPTIYHLREELVNSSEKADLRLVYLALAHIIKYRGNFLIEGALDTQNTSV | |
| | DGIYKQFIQTYNQVFASGIEDGSLKKLEDNKDVAKILVEKVTRKEKLERILKLYPGEKSA | |
| | GMFAQFISLIVGSKGNFQKPFDLIEKSDIECAKDSYEEDLESLLALIGDEYAELFVAAKN | |
| | AYSAVVLSSIITVAETETNAKLSASMIERFDTHEEDLGELKAFIKLHLPKHYEEIFSNTE | |
| | KHGYAGYIDGKTKQADFYKYMKMTLENIEGADYFIAKIEKENFLRKQRTFDNGAIPHQLH | |
| | LEELEAILHQQAKYYPFLKENYDKIKSLVTFRIPYFVGPLANGQSEFAWLTRKADGEIRP | |
| | WNIEEKVDFGKSAVDFIEKMTNKDTYLPKENVLPKHSLCYQKYLVYNELTKVRYINDQGK | |
| | TSYFSGQEKEQIFNDLFKQKRKVKKKDLELFLRNMSHVESPTIEGLEDSFNSSYSTYHDL | |
| | LKVGIKQEILDNPVNTEMLENIVKILTVFEDKRMIKEQLQQFSDVLDGVVLKKLERRHYT | |
| | GWGRLSAKLLMGIRDKQSHLTILDYLMNDDGLNRNLMQLINDSNLSFKSIIEKEQVTTAD | |
| | KDIQSIVADLAGSPAIKKGILQSLKIVDELVSVMGYPPQTIVVEMARENQTTGKGKNNSR | |
| | PRYKSLEKAIKEFGSQILKEHPTDNQELRNNRLYLYYLQNGKDMYTGQDLDIHNLSNYDI | |
| | DHIVPQSFITDNSIDNLVLTSSAGNREKGDDVPPLEIVRKRKVFWEKLYQGNLMSKRKFD | |
| | YLTKAERGGLTEADKARFIHRQLVETRQITKNVANILHQRFNYEKDDHGNTMKQVRIVTL | |
| | KSALVSQFRKQFQLYKVRDVNDYHHAHDAYLNGVVANTLLKVYPQLEPEFVYGDYHQFDW | |
| | FKANKATAKKQFYTNIMLFFAQKDRIIDENGEILWDKKYLDTVKKVMSYRQMNIVKKTEI | |
| | QKGEFSKATIKPKGNSSKLIPRKTNWDPMKYGGLDSPNMAYAVVIEYAKGKNKLVFEKKI | |
| | IRVTIMERKAFEKDEKAFLEEQGYRQPKVLAKLPKYTLYECEEGRRRMLASANEAQKGNQ | |
| | QVLPNHLVTLLHHAANCEVSDGKSLDYIESNREMFAELLAHVSEFAKRYTLAEANLNKIN | |
| | QLFEQNKEGDIKAIAQSFVDLMAFNAMGAPASFKFFETTIERKRYNNLKELLNSTIIYQS | |
| | ITGLYESRKRLDD | |
| 116 | MAAFKPNSINYILGLDIGIASVGWAMVEIDEEENPIRLIDLGVRVFERAEVPKTGDSLAM | Neisseria |
| | ARRLARSVRRLTRRRAHRLLRTRRLLKREGVLQAANFDENGLIKSLPNTPWQLRAAALDR | meningitidis |
| | KLTPLEWSAVLLHLIKHRGYLSQRKNEGETADKELGALLKGVAGNAHALQTGDFRTPAEL | Cas9 |
| | ALNKFEKESGHIRNQRSDYSHTFSRKDLQAELILLFEKQKEFGNPHVSGGLKEGIETLLM | |
| | TQRPALSGDAVQKMLGHCTFEPAEPKAAKNTYTAERFIWLTKLNNLRILEQGSERPLTDT | |
| | ERATLMDEPYRKSKLTYAQARKLLGLEDTAFFKGLRYGKDNAEASTLMEMKAYHAISRAL | |
| | EKEGLKDKKSPLNLSPELQDEIGTAFSLFKTDEDITGRLKDRIQPEILEALLKHISFDKF | |
| | VQISLKALRRIVPLMEQGKRYDEACAEIYGDHYGKKNTEEKIYLPPIPADEIRNPVVLRA | |
| | LSQARKVINGVVRRYGSPARIHIETAREVGKSFKDRKEIEKRQEENRKDREKAAAKFREY | |
| | FPNFVGEPKSKDILKLRLYEQQHGKCLYSGKEINLGRLNEKGYVEIDHALPFSRTWDDSF | |
| | NNKVLVLGSENQNKGNQTPYEYFNGKDNSREWQEFKARVETSRFPRSKKQRILLQKFDED | |
| | GFKERNLNDTRYVNRFLCQFVADRMRLTGKGKKRVFASNGQITNLLRGFWGLRKVRAEND | |
| | RHHALDAVVVACSTVAMQQKITRFVRYKEMNAFDGKTIDKETGEVLHQKTHFPQPWEFFA | |
| | QEVMIRVFGKPDGKPEFEEADTLEKLRTLLAEKLSSRPEAVHEYVTPLFVSRAPNRKMSG | |
| | QGHMETVKSAKRLDEGVSVLRVPLTQLKLKDLEKMVNREREPKLYEALKARLEAHKDDPA | |
| | KAFAEPFYKYDKAGNRTQQVKAVRVEQVQKTGVWVRNHNGIADNATMVRVDVFEKGDKYY | |
| | LVPIYSWQVAKGILPDRAVVQGKDEEDWQLIDDSFNFKFSLHPNDLVEVITKKARMFGYF | |
| | ASCHRGTGNINIRIHDLDHKIGKNGILEGIGVKTALSFQKYQIDELGKEIRPCRLKKRPP | |
| | VR | |
| 117 | MDKKYSIGLDIGTNSVGWAVITDEYKVPSKKFKVLGNTDRHSIKKNLIGALLFDSGETAE | Streptococcus |
| | ATRLKRTARRRYTRRKNRICYLQEIFSNEMAKVDDSFFHRLEESFLVEEDKKHERHPIFG | pyogenes Cas9 |
| | NIVDEVAYHEKYPTIYHLRKKLVDSTDKADLRLIYLALAHMIKFRGHFLIEGDLNPDNSD | |
| | VDKLFIQLVQTYNQLFEENPINASGVDAKAILSARLSKSRRLENLIAQLPGEKKNGLFGN | |
| | LIALSLGLTPNFKSNFDLAEDAKLQLSKDTYDDDLDNLLAQIGDQYADLFLAAKNLSDAI | |
| | LLSDILRVNTEITKAPLSASMIKRYDEHHQDLTLLKALVRQQLPEKYKEIFFDQSKNGYA | |
| | GYIDGGASQEEFYKFIKPILEKMDGTEELLVKLNREDLLRKQRTFDNGSIPHQIHLGELH | |
| | AILRRQEDFYPFLKDNREKIEKILTFRIPYYVGPLARGNSRFAWMTRKSEETITPWNFEE | |
| | VVDKGASAQSFIERMTNFDKNLPNEKVLPKHSLLYEYFTVYNELTKVKYVTEGMRKPAFL | |
| | SGEQKKAIVDLLFKTNRKVTVKQLKEDYFKKIECFDSVEISGVEDRFNASLGTYHDLLKI | |
| | IKDKDFLDNEENEDILEDIVLTLTLFEDREMIEERLKTYAHLFDDKVMKQLKRRRYTGWG | |
| | RLSRKLINGIRDKQSGKTILDFLKSDGFANRNFMQLIHDDSLTFKEDIQKAQVSGQGDSL | |
| | HEHIANLAGSPAIKKGILQTVKVVDELVKVMGRHKPENIVIEMARENQTTQKGQKNSRER | |
| | MKRIEEGIKELGSQILKEHPVENTQLQNEKLYLYYLQNGRDMYVDQELDINRLSDYDVDH | |
| | IVPQSFLKDDSIDNKVLTRSDKNRGKSDNVPSEEVVKKMKNYWRQLLNAKLITQRKFDNL | |
| | TKAERGGLSELDKAGFIKRQLVETRQITKHVAQILDSRMNTKYDENDKLIREVKVITLKS | |
| | KLVSDFRKDFQFYKVREINNYHHAHDAYLNAVVGTALIKKYPKLESEFVYGDYKVYDVRK | |
| | MIAKSEQEIGKATAKYFFYSNIMNFFKTEITLANGEIRKRPLIETNGETGEIVWDKGRDF | |
| | ATVRKVLSMPQVNIVKKTEVQTGGFSKESILPKRNSDKLIARKKDWDPKKYGGFDSPTVA | |
| | YSVLVVAKVEKGKSKKLKSVKELLGITIMERSSFEKNPIDFLEAKGYKEVKKDLIIKLPK | |
| | YSLFELENGRKRMLASAGELQKGNELALPSKYVNFLYLASHYEKLKGSPEDNEQKQLFVE | |
| | QHKHYLDEIIEQISEFSKRVILADANLDKVLSAYNKHRDKPIREQAENIIHLFTLTNLGA | |
| | PAAFKYFDTTIDRKRYTSTKEVLDATLIHQSITGLYETRIDLSQLGGD | |
| 118 | CGTGAGGCTCCGGTGCCCGTCAGTGGGCAGAGCGCACATCGCCCACAGTCCCCGAGAAGT | EF1alpha |
| | TGGGGGGAGGGGTCGGCAATTGAACCGGTGCCTAGAGAAGGTGGCGCGGGGTAAACTGGG | promoter |
| | AAAGTGATGTCGTGTACTGGCTCCGCCTTTTTCCCGAGGGTGGGGGAGAACCGTATATAA | |
| | GTGCACTAGTCGCCGTGAACGTTCTTTTTCGCAACGGGTTTGCCGCCAGAACACAGGTAA | |
| | GTGCCGTGTGTGGTTCCCGCGGGCCTGGCCTCTTTACGGGTTATGGCCCTTGCGTGCCTT | |
| | GAATTACTTCCACCTGGCTGCAGTACGTGATTCTTGATCCCGAGCTTCGGGTTGGAAGTG | |
| | GGTGGGAGAGTTCGTGGCCTTGCGCTTAAGGAGCCCCTTCGCCTCGTGCTTGAGTTGTGG | |
| | CCTGGCCTGGGCGCTGGGGCCGCCGCGTGCGAATCTGGTGGCACCTTCGCGCCTGTCTCG | |
| | CTGCTTTCGATAAGTCTCTAGCCATTTAAAATTTTTGATGACCTGCTGCGACGCTTTTTT | |
| | TCTGGCAAGATAGTCTTGTAAATGCGGGCCAAGATCAGCACACTGGTATTTCGGTTTTTG | |
| | GGGCCGCGGGCGGCGACGGGGCCCGTGCGTCCCAGCGCACATGTTCGGCGAGGCGGGGCC | |
| | TGCGAGCGCGGCCACCGAGAATCGGACGGGGGTAGTCTCAAGCTGCCCGGCCTGCTCTGG | |
| | TGCCTGGCCTCGCGCCGCCGTGTATCGCCCCGCCCTGGGCGGCAAGGCTGGCCCGGTCGG | |
| | CACCAGTTGCGTGAGCGGAAAGATGGCCGCTTCCCGGCCCTGCTGCAGGGAGCACAAAAT | |
| | GGAGGACGCGGCGCTCGGGAGAGCGGGCGGGTGAGTCACCCACACAAAGGAAAAGGGCCT | |
| | TTCCGTCCTCAGCCGTCGCTTCATGTGACTCCACGGAGTACCGGGCGCCGTCCAGGCACC | |
| | TCGATTAGTTCTCCAGCTTTTGGAGTACGTCGTCTTTAGGTTGGGGGGAGGGGTTTTATG | |
| | CGATGGAGTTTCCCCACACTGAGTGGGTGGAGACTGAAGTTAGGCCAGCTTGGCACTTGA | |
| | TGTAATTCTCCTTGGAATTTGCCCTTTTTGAGTTTGGATCTTGGTTCATTCTCAAGCCTC | |
| | AGACAGTGGTTCAAAGTTTTTTTCTTCCATTTCAGGTGTCGTGAAAACTACCCCTAAAAG | |
| | CCAAA | |
| 119 | GGATCTGCGATCGCTCCGGTGCCCGTCAGTGGGCAGAGCGCACATCGCCCACAGTCCCCG | Ef1alpha |
| | AGAAGTTGGGGGGAGGGGTCGGCAATTGAACCGGTGCCTAGAGAAGGTGGCGCGGGGTAA | promoter with |
| | ACTGGGAAAGTGATGTCGTGTACTGGCTCCGCCTTTTTCCCGAGGGTGGGGGAGAACCGT | HTLV1 |
| | ATATAAGTGCAGTAGTCGCCGTGAACGTTCTTTTTCGCAACGGGTTTGCCGCCAGAACAC | enhancer |
| | AGCTGAAGCTTCGAGGGGCTCGCATCTCTCCTTCACGCGCCCGCCGCCCTACCTGAGGCC | |
| | GCCATCCACGCCGGTTGAGTCGCGTTCTGCCGCCTCCCGCCTGTGGTGCCTCCTGAACTG | |
| | CGTCCGCCGTCTAGGTAAGTTTAAAGCTCAGGTCGAGACCGGGCCTTTGTCCGGCGCTCC | |
| | CTTGGAGCCTACCTAGACTCAGCCGGCTCTCCACGCTTTGCCTGACCCTGCTTGCTCAAC | |
| | TCTACGTCTTTGTTTCGTTTTCTGTTCTGCGCCGTTACAGATCCAAGCTGTGACCGGCGC | |
| | CTAC | |
| 120 | GGATCGGAGCGACGAATTTTAGTCTACTGAAACAAGCGGGAGACGTGGAGGAAAACCCT | P2A nucleotide |
| | GGACCT | sequence |
| 121 | atggataaaaagtacagcatcgggctggacatcggtacaaactcagtggggtgggccgtg | S. pyogenes |
| | attacggacgagtacaaggtaccctccaaaaaatttaaagtgctgggtaacacggacaga | Cas9 codon |
| | cactctataaagaaaaatcttattggagccttgctgttcgactcaggcgagacagccgaa | optimized |
| | gccacaaggttgaagcggaccgccaggaggcggtataccaggagaaagaaccgcatatgc | nucleic acid |
| | tacctgcaagaaatcttcagtaacgagatggcaaaggttgacgatagctttttccatcgc | sequence |
| | ctggaagaatcctttcttgttgaggaagacaagaagcacgaacggcaccccatctttggc | |
| | aatattgtcgacgaagtggcatatcacgaaaagtacccgactatctaccacctcaggaag | |
| | aagctggtggactctaccgataaggcggacctcagacttatttatttggcactcgcccac | |
| | atgattaaatttagaggacatttcttgatcgagggcgacctgaacccggacaacagtgac | |
| | gtcgataagctgttcatccaacttgtgcagacctacaatcaactgttcgaagaaaaccct | |
| | ataaatgcttcaggagtcgacgctaaagcaatcctgtccgcgcgcctctcaaaatctaga | |
| | agacttgagaatctgattgctcagttgcccggggaaaagaaaaatggattgtttggcaac | |
| | ctgatcgccctcagtctcggactgaccccaaatttcaaaagtaacttcgacctggccgaa | |
| | gacgctaagctccagctgtccaaggacacatacgatgacgacctcgacaatctgctggcc | |
| | cagattggggatcagtacgccgatctctttttggcagcaaagaacctgtccgacgccatc | |
| | ctgttgagcgatatcttgagagtgaacaccgaaattactaaagcaccccttagcgcatct | |
| | atgatcaagcggtacgacgagcatcatcaggatctgaccctgctgaaggctcttgtgagg | |
| | caacagctccccgaaaaatacaaggaaatcttctttgaccagagcaaaaacggctacgct | |
| | ggctatatagatggtggggccagtcaggaggaattctataaattcatcaagcccattctc | |
| | gagaaaatggacggcacagaggagttgctggtcaaacttaacagggaggacctgctgcgg | |
| | aagcagcggacctttgacaacgggtctatcccccaccagattcatctgggcgaactgcac | |
| | gcaatcctgaggaggcaggaggatttttatccttttcttaaagataaccgcgagaaaata | |
| | gaaaagattcttacattcaggatcccgtactacgtgggacctctcgcccggggcaattca | |
| | cggtttgcctggatgacaaggaagtcagaggagactattacaccttggaacttcgaagaa | |
| | gtggtggacaagggtgcatctgcccagtctttcatcgagcggatgacaaattttgacaag | |
| | aacctccctaatgagaaggtgctgcccaaacattctctgctctacgagtactttaccgtc | |
| | tacaatgaactgactaaagtcaagtacgtcaccgagggaatgaggaagccggcattcctt | |
| | agtggagaacagaagaaggcgattgtagacctgttgttcaagaccaacaggaaggtgact | |
| | gtgaagcaacttaaagaagactactttaagaagatcgaatgttttgacagtgtggaaatt | |
| | tcaggggttgaagaccgcttcaatgcgtcattggggacttaccatgatcttctcaagatc | |
| | ataaaggacaaagacttcctggacaacgaagaaaatgaggatattctcgaagacatcgtc | |
| | ctcaccctgaccctgttcgaagacagggaaatgatagaagagcgcttgaaaacctatgcc | |
| | cacctcttcgacgataaagttatgaagcagctgaagcgcaggagatacacaggatgggga | |
| | agattgtcaaggaagctgatcaatggaattagggataaacagagtggcaagaccatactg | |
| | gatttcctcaaatctgatggcttcgccaataggaacttcatgcaactgattcacgatgac | |
| | tctcttaccttcaaggaggacattcaaaaggctcaggtgagcgggcagggagactccctt | |
| | catgaacacatcgcgaatttggcaggttcccccgctattaaaaagggcatccttcaaact | |
| | gtcaaggtggtggatgaattggtcaaggtaatgggcagacataagccagaaaatattgtg | |
| | atcgagatggcccgcgaaaaccagaccacacagaagggccagaaaaatagtagagagcgg | |
| | atgaagaggatcgaggagggcatcaaagagctgggatctcagattctcaaagaacacccc | |
| | gtagaaaacacacagctgcagaacgaaaaattgtacttgtactatctgcagaacggcaga | |
| | gacatgtacgtcgaccaagaacttgatattaatagactgtccgactatgacgtagaccat | |
| | atcgtgccccagtccttcctgaaggacgactccattgataacaaagtcttgacaagaagc | |
| | gacaagaacaggggtaaaagtgataatgtgcctagcgaggaggtggtgaaaaaaatgaag | |
| | aactactggcgacagctgcttaatgcaaagctcattacacaacggaagttcgataatctg | |
| | acgaaagcagagagaggtggcttgtctgagttggacaaggcagggtttattaagcggcag | |
| | ctggtggaaactaggcagatcacaaagcacgtggcgcagattttggacagccggatgaac | |
| | acaaaatacgacgaaaatgataaactgatacgagaggtcaaagttatcacgctgaaaagc | |
| | aagctggtgtccgattttcggaaagacttccagttctacaaagttcgcgagattaataac | |
| | taccatcatgctcacgatgcgtacctgaacgctgttgtcgggaccgccttgataaagaag | |
| | tacccaaagctggaatccgagttcgtatacggggattacaaagtgtacgatgtgaggaaa | |
| | atgatagccaagtccgagcaggagattggaaaggccacagctaagtacttcttttattct | |
| | aacatcatgaatttttttaagacggaaattaccctggccaacggagagatcagaaagcgg | |
| | ccccttatagagacaaatggtgaaacaggtgaaatcgtctgggataagggcagggatttc | |
| | gctactgtgaggaaggtgctgagtatgccacaggtaaatatcgtgaaaaaaaccgaagta | |
| | cagaccggaggattttccaaggaaagcattttgcctaaaagaaactcagacaagctcatc | |
| | gcccgcaagaaagattgggaccctaagaaatacgggggatttgactcacccaccgtagcc | |
| | tattctgtgctggtggtagctaaggtggaaaaaggaaagtctaagaagctgaagtccgtg | |
| | aaggaactcttgggaatcactatcatggaaagatcatcctttgaaaagaaccctatcgat | |
| | ttcctggaggctaagggttacaaggaggtcaagaaagacctcatcattaaactgccaaaa | |
| | tactctctcttcgagctggaaaatggcaggaagagaatgttggccagcgccggagagctg | |
| | caaaagggaaacgagcttgctctgccctccaaatatgttaattttctctatctcgcttcc | |
| | cactatgaaaagctgaaagggtctcccgaagataacgagcagaagcagctgttcgtcgaa | |
| | cagcacaagcactatctggatgaaataatcgaacaaataagcgagttcagcaaaagggtt | |
| | atcctggcggatgctaatttggacaaagtactgtctgcttataacaagcaccgggataag | |
| | cctattagggaacaagccgagaatataattcacctctttacactcacgaatctcggagcc | |
| | cccgccgccttcaaatactttgatacgactatcgaccggaaacggtataccagtaccaaa | |
| | gaggtcctcgatgccaccctcatccaccagtcaattactggcctgtacgaaacacggatc | |
| | gacctctctcaactgggcggcgactag | |
| 122 | MDKKYSIGLDIGTNSVGWAVITDEYKVPSKKFKVLGNTDRHSIKKNLIGALLFDSGETAE | S. pyogenes |
| | ATRLKRTARRRYTRRKNRICYLQEIFSNEMAKVDDSFFHRLEESFLVEEDKKHERHPIFG | Cas9 |
| | NIVDEVAYHEKYPTIYHLRKKLVDSTDKADLRLIYLALAHMIKFRGHFLIEGDLNPDNSD | |
| | VDKLFIQLVQTYNQLFEENPINASGVDAKAILSARLSKSRRLENLIAQLPGEKKNGLFGN | |
| | LIALSLGLTPNFKSNFDLAEDAKLQLSKDTYDDDLDNLLAQIGDQYADLFLAAKNLSDAI | |
| | LLSDILRVNTEITKAPLSASMIKRYDEHHQDLTLLKALVRQQLPEKYKEIFFDQSKNGYA | |
| | GYIDGGASQEEFYKFIKPILEKMDGTEELLVKLNREDLLRKQRTFDNGSIPHQIHLGELH | |
| | AILRRQEDFYPFLKDNREKIEKILTFRIPYYVGPLARGNSRFAWMTRKSEETITPWNFEE | |
| | VVDKGASAQSFIERMTNFDKNLPNEKVLPKHSLLYEYFTVYNELTKVKYVTEGMRKPAFL | |
| | SGEQKKAIVDLLFKTNRKVTVKQLKEDYFKKIECFDSVEISGVEDRFNASLGTYHDLLKI | |
| | IKDKDFLDNEENEDILEDIVLTLTLFEDREMIEERLKTYAHLFDDKVMKQLKRRRYTGWG | |
| | RLSRKLINGIRDKQSGKTILDFLKSDGFANRNFMQLIHDDSLTFKEDIQKAQVSGQGDSL | |
| | HEHIANLAGSPAIKKGILQTVKVVDELVKVMGRHKPENIVIEMARENQTTQKGQKNSRER | |
| | MKRIEEGIKELGSQILKEHPVENTQLQNEKLYLYYLQNGRDMYVDQELDINRLSDYDVDH | |
| | IVPQSFLKDDSIDNKVLTRSDKNRGKSDNVPSEEVVKKMKNYWRQLLNAKLITQRKFDNL | |
| | TKAERGGLSELDKAGFIKRQLVETRQITKHVAQILDSRMNTKYDENDKLIREVKVITLKS | |
| | KLVSDFRKDFQFYKVREINNYHHAHDAYLNAVVGTALIKKYPKLESEFVYGDYKVYDVRK | |
| | MIAKSEQEIGKATAKYFFYSNIMNFFKTEITLANGEIRKRPLIETNGETGEIVWDKGRDF | |
| | ATVRKVLSMPQVNIVKKTEVQTGGFSKESILPKRNSDKLIARKKDWDPKKYGGFDSPTVA | |
| | YSVLVVAKVEKGKSKKLKSVKELLGITIMERSSFEKNPIDFLEAKGYKEVKKDLIIKLPK | |
| | YSLFELENGRKRMLASAGELQKGNELALPSKYVNFLYLASHYEKLKGSPEDNEQKQLFVE | |
| | QHKHYLDEIIEQISEFSKRVILADANLDKVLSAYNKHRDKPIREQAENIIHLFTLTNLGA | |
| | PAAFKYFDTTIDRKRYTSTKEVLDATLIHQSITGLYETRIDLSQLGGD | |
| 123 | atggccgccttcaagcccaaccccatcaactacatcctgggcctggacatcggcatcgcc | N. meningitidis |
| | agcgtgggctgggccatggtggagatcgacgaggacgagaaccccatctgcctgatcgac | Cas9 codon |
| | ctgggtgtgcgcgtgttcgagcgcgctgaggtgcccaagactggtgacagtctggctatg | optimized |
| | gctcgccggcttgctcgctctgttcggcgccttactcgccggcgcgctcaccgccttctg | nucleic acid |
| | cgcgctcgccgcctgctgaagcgcgagggtgtgctgcaggctgccgacttcgacgagaac | sequence |
| | ggcctgatcaagagcctgcccaacactccttggcagctgcgcgctgccgctctggaccgc | |
| | aagctgactcctctggagtggagcgccgtgctgctgcacctgatcaagcaccgcggctac | |
| | ctgagccagcgcaagaacgagggcgagaccgccgacaaggagctgggtgctctgctgaag | |
| | ggcgtggccgacaacgcccacgccctgcagactggtgacttccgcactcctgctgagctg | |
| | gccctgaacaagttcgagaaggagagcggccacatccgcaaccagcgcggcgactacagc | |
| | cacaccttcagccgcaaggacctgcaggccgagctgatcctgctgttcgagaagcagaag | |
| | gagttcggcaacccccacgtgagcggcggcctgaaggagggcatcgagaccctgctgatg | |
| | acccagcgccccgccctgagcggcgacgccgtgcagaagatgctgggccactgcaccttc | |
| | gagccagccgagcccaaggccgccaagaacacctacaccgccgagcgcttcatctggctg | |
| | accaagctgaacaacctgcgcatcctggagcagggcagcgagcgccccctgaccgacacc | |
| | gagcgcgccaccctgatggacgagccctaccgcaagagcaagctgacctacgcccaggcc | |
| | cgcaagctgctgggtctggaggacaccgccttcttcaagggcctgcgctacggcaaggac | |
| | aacgccgaggccagcaccctgatggagatgaaggcctaccacgccatcagccgcgccctg | |
| | gagaaggagggcctgaaggacaagaagagtcctctgaacctgagccccgagctgcaggac | |
| | gagatcggcaccgccttcagcctgttcaagaccgacgaggacatcaccggccgcctgaag | |
| | gaccgcatccagcccgagatcctggaggccctgctgaagcacatcagcttcgacaagttc | |
| | gtgcagatcagcctgaaggccctgcgccgcatcgtgcccctgatggagcagggcaagcgc | |
| | tacgacgaggcctgcgccgagatctacggcgaccactacggcaagaagaacaccgaggag | |
| | aagatctacctgcctcctatccccgccgacgagatccgcaaccccgtggtgctgcgcgcc | |
| | ctgagccaggcccgcaaggtgatcaacggcgtggtgcgccgctacggcagccccgcccgc | |
| | atccacatcgagaccgcccgcgaggtgggcaagagcttcaaggaccgcaaggagatcgag | |
| | aagcgccaggaggagaaccgcaaggaccgcgagaaggccgccgccaagttccgcgagtac | |
| | ttccccaacttcgtgggcgagcccaagagcaaggacatcctgaagctgcgcctgtacgag | |
| | cagcagcacggcaagtgcctgtacagcggcaaggagatcaacctgggccgcctgaacgag | |
| | aagggctacgtggagatcgaccacgccctgcccttcagccgcacctgggacgacagcttc | |
| | aacaacaaggtgctggtgctgggcagcgagaaccagaacaagggcaaccagaccccctac | |
| | gagtacttcaacggcaaggacaacagccgcgagtggcaggagttcaaggcccgcgtggag | |
| | accagccgcttcccccgcagcaagaagcagcgcatcctgctgcagaagttcgacgaggac | |
| | ggcttcaaggagcgcaacctgaacgacacccgctacgtgaaccgcttcctgtgccagttc | |
| | gtggccgaccgcatgcgcctgaccggcaagggcaagaagcgcgtgttcgccagcaacggc | |
| | cagatcaccaacctgctgcgcggcttctggggcctgcgcaaggtgcgcgccgagaacgac | |
| | cgccaccacgccctggacgccgtggtggtggcctgcagcaccgtggccatgcagcagaag | |
| | atcacccgcttcgtgcgctacaaggagatgaacgccttcgacggtaaaaccatcgacaag | |
| | gagaccggcgaggtgctgcaccagaagacccacttcccccagccctgggagttcttcgcc | |
| | caggaggtgatgatccgcgtgttcggcaagcccgacggcaagcccgagttcgaggaggcc | |
| | gacacccccgagaagctgcgcaccctgctggccgagaagctgagcagccgccctgaggcc | |
| | gtgcacgagtacgtgactcctctgttcgtgagccgcgcccccaaccgcaagatgagcggt | |
| | cagggtcacatggagaccgtgaagagcgccaagcgcctggacgagggcgtgagcgtgctg | |
| | cgcgtgcccctgacccagctgaagctgaaggacctggagaagatggtgaaccgcgagcgc | |
| | gagcccaagctgtacgaggccctgaaggcccgcctggaggcccacaaggacgaccccgcc | |
| | aaggccttcgccgagcccttctacaagtacgacaaggccggcaaccgcacccagcaggtg | |
| | aaggccgtgcgcgtggagcaggtgcagaagaccggcgtgtgggtgcgcaaccacaacggc | |
| | atcgccgacaacgccaccatggtgcgcgtggacgtgttcgagaagggcgacaagtactac | |
| | ctggtgcccatctacagctggcaggtggccaagggcatcctgcccgaccgcgccgtggtg | |
| | cagggcaaggacgaggaggactggcagctgatcgacgacagcttcaacttcaagttcagc | |
| | ctgcaccccaacgacctggtggaggtgatcaccaagaaggcccgcatgttcggctacttc | |
| | gccagctgccaccgcggcaccggcaacatcaacatccgcatccacgacctggaccacaag | |
| | atcggcaagaacggcatcctggagggcatcggcgtgaagaccgccctgagcttccagaag | |
| | taccagatcgacgagctgggcaaggagatccgcccctgccgcctgaagaagcgccctcct | |
| | gtgcgctaa | |
| 124 | MAAFKPNPINYILGLDIGIASVGWAMVEIDEDENPICLIDLGVRVFERAEVPKTGDSLAM | N. meningitidis |
| | ARRLARSVRRLTRRRAHRLLRARRLLKREGVLQAADFDENGLIKSLPNTPWQLRAAALDR | Cas9 |
| | KLTPLEWSAVLLHLIKHRGYLSQRKNEGETADKELGALLKGVADNAHALQTGDFRTPAEL | |
| | ALNKFEKESGHIRNQRGDYSHTFSRKDLQAELILLFEKQKEFGNPHVSGGLKEGIETLLM | |
| | TQRPALSGDAVQKMLGHCTFEPAEPKAAKNTYTAERFIWLTKLNNLRILEQGSERPLTDT | |
| | ERATLMDEPYRKSKLTYAQARKLLGLEDTAFFKGLRYGKDNAEASTLMEMKAYHAISRAL | |
| | EKEGLKDKKSPLNLSPELQDEIGTAFSLFKTDEDITGRLKDRIQPEILEALLKHISFDKF | |
| | VQISLKALRRIVPLMEQGKRYDEACAEIYGDHYGKKNTEEKIYLPPIPADEIRNPVVLRA | |
| | LSQARKVINGVVRRYGSPARIHIETAREVGKSFKDRKEIEKRQEENRKDREKAAAKFREY | |
| | FPNFVGEPKSKDILKLRLYEQQHGKCLYSGKEINLGRLNEKGYVEIDHALPFSRTWDDSF | |
| | NNKVLVLGSENQNKGNQTPYEYFNGKDNSREWQEFKARVETSRFPRSKKQRILLQKFDED | |
| | GFKERNLNDTRYVNRFLCQFVADRMRLTGKGKKRVFASNGQITNLLRGFWGLRKVRAEND | |
| | RHHALDAVVVACSTVAMQQKITRFVRYKEMNAFDGKTIDKETGEVLHQKTHFPQPWEFFA | |
| | QEVMIRVFGKPDGKPEFEEADTPEKLRTLLAEKLSSRPEAVHEYVTPLFVSRAPNRKMSG | |
| | QGHMETVKSAKRLDEGVSVLRVPLTQLKLKDLEKMVNREREPKLYEALKARLEAHKDDPA | |
| | KAFAEPFYKYDKAGNRTQQVKAVRVEQVQKTGVWVRNHNGIADNATMVRVDVFEKGDKYY | |
| | LVPIYSWQVAKGILPDRAVVQGKDEEDWQLIDDSFNFKFSLHPNDLVEVITKKARMFGYF | |
| | ASCHRGTGNINIRIHDLDHKIGKNGILEGIGVKTALSFQKYQIDELGKEIRPCRLKKRPP | |
| | VR | |
| 125 | atgaaaaggaactacattctggggctggacatcgggattacaagcgtggggtatgggatt | S. aureus Cas9 |
| | attgactatgaaacaagggacgtgatcgacgcaggcgtcagactgttcaaggaggccaac | codon |
| | gtggaaaacaatgagggacggagaagcaagaggggagccaggcgcctgaaacgacggaga | optimized |
| | aggcacagaatccagagggtgaagaaactgctgttcgattacaacctgctgaccgaccat | nucleic acid |
| | tctgagctgagtggaattaatccttatgaagccagggtgaaaggcctgagtcagaagctg | sequence |
| | tcagaggaagagttttccgcagctctgctgcacctggctaagcgccgaggagtgcataac | |
| | gtcaatgaggtggaagaggacaccggcaacgagctgtctacaaaggaacagatctcacgc | |
| | aatagcaaagctctggaagagaagtatgtcgcagagctgcagctggaacggctgaagaaa | |
| | gatggcgaggtgagagggtcaattaataggttcaagacaagcgactacgtcaaagaagcc | |
| | aagcagctgctgaaagtgcagaaggcttaccaccagctggatcagagcttcatcgatact | |
| | tatatcgacctgctggagactcggagaacctactatgagggaccaggagaagggagcccc | |
| | ttcggatggaaagacatcaaggaatggtacgagatgctgatgggacattgcacctatttt | |
| | ccagaagagctgagaagcgtcaagtacgcttataacgcagatctgtacaacgccctgaat | |
| | gacctgaacaacctggtcatcaccagggatgaaaacgagaaactggaatactatgagaag | |
| | ttccagatcatcgaaaacgtgtttaagcagaagaaaaagcctacactgaaacagattgct | |
| | aaggagatcctggtcaacgaagaggacatcaagggctaccgggtgacaagcactggaaaa | |
| | ccagagttcaccaatctgaaagtgtatcacgatattaaggacatcacagcacggaaagaa | |
| | atcattgagaacgccgaactgctggatcagattgctaagatcctgactatctaccagagc | |
| | tccgaggacatccaggaagagctgactaacctgaacagcgagctgacccaggaagagatc | |
| | gaacagattagtaatctgaaggggtacaccggaacacacaacctgtccctgaaagctatc | |
| | aatctgattctggatgagctgtggcatacaaacgacaatcagattgcaatctttaaccgg | |
| | ctgaagctggtcccaaaaaaggtggacctgagtcagcagaaagagatcccaaccacactg | |
| | gtggacgatttcattctgtcacccgtggtcaagcggagcttcatccagagcatcaaagtg | |
| | atcaacgccatcatcaagaagtacggcctgcccaatgatatcattatcgagctggctagg | |
| | gagaagaacagcaaggacgcacagaagatgatcaatgagatgcagaaacgaaaccggcag | |
| | accaatgaacgcattgaagagattatccgaactaccgggaaagagaacgcaaagtacctg | |
| | attgaaaaaatcaagctgcacgatatgcaggagggaaagtgtctgtattctctggaggcc | |
| | atccccctggaggacctgctgaacaatccattcaactacgaggtcgatcatattatcccc | |
| | agaagcgtgtccttcgacaattcctttaacaacaaggtgctggtcaagcaggaagagaac | |
| | tctaaaaagggcaataggactcctttccagtacctgtctagttcagattccaagatctct | |
| | tacgaaacctttaaaaagcacattctgaatctggccaaaggaaagggccgcatcagcaag | |
| | accaaaaaggagtacctgctggaagagcgggacatcaacagattctccgtccagaaggat | |
| | tttattaaccggaatctggtggacacaagatacgctactcgcggcctgatgaatctgctg | |
| | cgatcctatttccgggtgaacaatctggatgtgaaagtcaagtccatcaacggcgggttc | |
| | acatcttttctgaggcgcaaatggaagtttaaaaaggagcgcaacaaagggtacaagcac | |
| | catgccgaagatgctctgattatcgcaaatgccgacttcatctttaaggagtggaaaaag | |
| | ctggacaaagccaagaaagtgatggagaaccagatgttcgaagagaagcaggccgaatct | |
| | atgcccgaaatcgagacagaacaggagtacaaggagattttcatcactcctcaccagatc | |
| | aagcatatcaaggatttcaaggactacaagtactctcaccgggtggataaaaagcccaac | |
| | agagagctgatcaatgacaccctgtatagtacaagaaaagacgataaggggaataccctg | |
| | attgtgaacaatctgaacggactgtacgacaaagataatgacaagctgaaaaagctgatc | |
| | aacaaaagtcccgagaagctgctgatgtaccaccatgatcctcagacatatcagaaactg | |
| | aagctgattatggagcagtacggcgacgagaagaacccactgtataagtactatgaagag | |
| | actgggaactacctgaccaagtatagcaaaaaggataatggccccgtgatcaagaagatc | |
| | aagtactatgggaacaagctgaatgcccatctggacatcacagacgattaccctaacagt | |
| | cgcaacaaggtggtcaagctgtcactgaagccatacagattcgatgtctatctggacaac | |
| | ggcgtgtataaatttgtgactgtcaagaatctggatgtcatcaaaaaggagaactactat | |
| | gaagtgaatagcaagtgctacgaagaggctaaaaagctgaaaaagattagcaaccaggca | |
| | gagttcatcgcctccttttacaacaacgacctgattaagatcaatggcgaactgtatagg | |
| | gtcatcggggtgaacaatgatctgctgaaccgcattgaagtgaatatgattgacatcact | |
| | taccgagagtatctggaaaacatgaatgataagcgcccccctcgaattatcaaaacaatt | |
| | gcctctaagactcagagtatcaaaaagtactcaaccgacattctgggaaacctgtatgag | |
| | gtgaagagcaaaaagcaccctcagattatcaaaaagggc | |
| 126 | MKRNYILGLDIGITSVGYGIIDYETRDVIDAGVRLFKEANVENNEGRRSKRGARRLKRRR | S. aureus Cas9 |
| | RHRIQRVKKLLFDYNLLTDHSELSGINPYEARVKGLSQKLSEEEFSAALLHLAKRRGVHN | |
| | VNEVEEDTGNELSTKEQISRNSKALEEKYVAELQLERLKKDGEVRGSINRFKTSDYVKEA | |
| | KQLLKVQKAYHQLDQSFIDTYIDLLETRRTYYEGPGEGSPFGWKDIKEWYEMLMGHCTYF | |
| | PEELRSVKYAYNADLYNALNDLNNLVITRDENEKLEYYEKFQIIENVFKQKKKPTLKQIA | |
| | KEILVNEEDIKGYRVTSTGKPEFTNLKVYHDIKDITARKEIIENAELLDQIAKILTIYQS | |
| | SEDIQEELTNLNSELTQEEIEQISNLKGYTGTHNLSLKAINLILDELWHTNDNQIAIFNR | |
| | LKLVPKKVDLSQQKEIPTTLVDDFILSPVVKRSFIQSIKVINAIIKKYGLPNDIIIELAR | |
| | EKNSKDAQKMINEMQKRNRQTNERIEEIIRTTGKENAKYLIEKIKLHDMQEGKCLYSLEA | |
| | IPLEDLLNNPFNYEVDHIIPRSVSEDNSENNKVLVKQEENSKKGNRTPFQYLSSSDSKIS | |
| | YETFKKHILNLAKGKGRISKTKKEYLLEERDINRFSVQKDFINRNLVDTRYATRGLMNLL | |
| | RSYFRVNNLDVKVKSINGGFTSFLRRKWKFKKERNKGYKHHAEDALIIANADFIFKEWKK | |
| | LDKAKKVMENQMFEEKQAESMPEIETEQEYKEIFITPHQIKHIKDFKDYKYSHRVDKKPN | |
| | RELINDTLYSTRKDDKGNTLIVNNLNGLYDKDNDKLKKLINKSPEKLLMYHHDPQTYQKL | |
| | KLIMEQYGDEKNPLYKYYEETGNYLTKYSKKDNGPVIKKIKYYGNKLNAHLDITDDYPNS | |
| | RNKVVKLSLKPYREDVYLDNGVYKEVTVKNLDVIKKENYYEVNSKCYEEAKKLKKISNQA | |
| | EFIASFYNNDLIKINGELYRVIGVNNDLLNRIEVNMIDITYREYLENMNDKRPPRIIKTI | |
| | ASKTQSIKKYSTDILGNLYEVKSKKHPQIIKKG | |
| 127 | ATTGCACTCATCAGAGCTAC | TGFBR2 target |
| | | sequence 9 |
| 128 | CCTAGAGTGAAGAGATTCAT | TGFBR2 target |
| | | sequence 10 |
| 129 | CCAATGAATCTCTTCACTCT | TGFBR2 target |
| | | sequence 11 |
| 130 | AAAGTCATGGTAGGGGAGCT | TGFBR2 target |
| | | sequence 12 |
| 131 | GTGAGCAATCCCCCGGGCGA | TGFBR2 target |
| | | sequence 13 |
| 132 | GTCGTTCTTCACGAGGATAT | TGFBR2 target |
| | | sequence 14 |
| 133 | GCCGCGTCAGGTACTCCTGT | TGFBR2 target |
| | | sequence 15 |
| 134 | GACGCGGCATGTCATCAGCT | TGFBR2 target |
| | | sequence 16 |
| 135 | GCTTCTGCTGCCGGTTAACG | TGFBR2 target |
| | | sequence 17 |
| 136 | GTGGATGACCTGGCTAACAG | TGFBR2 target |
| | | sequence 18 |
| 137 | GTGATCACACTCCATGTGGG | TGFBR2 target |
| | | sequence 19 |
| 138 | GCCCATTGAGCTGGACACCC | TGFBR2 target |
| | | sequence 20 |
| 139 | GCGGTCATCTTCCAGGATGA | TGFBR2 target |
| | | sequence 21 |
| 140 | GGGAGCTGCCCAGCTTGCGC | TGFBR2 target |
| | | sequence 22 |
| 141 | GTTGATGTTGTTGGCACACG | TGFBR2 target |
| | | sequence 23 |
| 142 | GGCATCTTGGGCCTCCCACA | TGFBR2 target |
| | | sequence 24 |
| 143 | GCGGCATGTCATCAGCTGGG | TGFBR2 target |
| | | sequence 25 |
| 144 | GCTCCTCAGCCGTCAGGAAC | TGFBR2 target |
| | | sequence 26 |
| 145 | GCTGGTGTTATATTCTGATG | TGFBR2 target |
| | | sequence 27 |
| 146 | CCGACTTCTGAACGTGCGGT | TGFBR2 target |
| | | sequence 28 |
| 147 | TGCTGGCGATACGCGTCCAC | TGFBR2 target |
| | | sequence 29 |
| 148 | CCCGACTTCTGAACGTGCGG | TGFBR2 target |
| | | sequence 30 |
| 149 | CCACCGCACGTTCAGAAGTC | TGFBR2 target |
| | | sequence 31 |
| 150 | TCACCCGACTTCTGAACGTG | TGFBR2 target |
| | | sequence 32 |
| 151 | CCCACCGCACGTTCAGAAGT | TGFBR2 target |
| | | sequence 33 |
| 152 | CGAGCAGCGGGGTCTGCCAT | TGFBR2 target |
| | | sequence 34 |
| 153 | ACGAGCAGCGGGGTCTGCCA | TGFBR2 target |
| | | sequence 35 |
| 154 | AGCGGGGTCTGCCATGGGTC | TGFBR2 target |
| | | sequence 36 |
| 155 | CCTGAGCAGCCCCCGACCCA | TGFBR2 target |
| | | sequence 37 |
| 156 | AACGTGCGGTGGGATCGTGC | TGFBR2 target |
| | | sequence 38 |
| 157 | GGACGATGTGCAGCGGCCAC | TGFBR2 target |
| | | sequence 39 |
| 158 | GTCCACAGGACGATGTGCAG | TGFBR2 target |
| | | 1sequence 40 |
| 159 | CATGGGTCGGGGGCTGCTCA | TGFBR2 target |
| | | sequence 41 |
| 160 | CCATGGGTCGGGGGCTGCTC | TGFBR2 target |
| | | sequence 42 |
| 161 | CAGCGGGGTCTGCCATGGGT | TGFBR2 target |
| | | sequence 43 |
| 162 | ATGGGTCGGGGGCTGCTCAG | TGFBR2 target |
| | | sequence 44 |
| 163 | CGGGGTCTGCCATGGGTCGG | TGFBR2 target |
| | | sequence 45 |
| 164 | AGGAAGTCTGTGTGGCTGTA | TGFBR2 target |
| | | sequence 46 |
| 165 | CTCCATCTGTGAGAAGCCAC | TGFBR2 target |
| | | sequence 47 |
| 166 | ATGATAGTCACTGACAACAA | TGFBR2 target |
| | | sequence 48 |
| 167 | GATGCTGCAGTTGCTCATGC | TGFBR2 target |
| | | sequence 49 |
| 168 | ACAGCCACACAGACTTCCTG | TGFBR2 target |
| | | sequence 50 |
| 169 | GAAGCCACAGGAAGTCTGTG | TGFBR2 target |
| | | sequence 51 |
| 170 | TTCCTGTGGCTTCTCACAGA | TGFBR2 target |
| | | sequence 52 |
| 171 | CTGTGGCTTCTCACAGATGG | TGFBR2 target |
| | | sequence 53 |
| 172 | TCACAAAATTTACACAGTTG | TGFBR2 target |
| | | sequence 54 |
| 173 | CCCCTACCATGACTTTATTC | TGFBR2 target |
| | | sequence 55 |
| 174 | CCAGAATAAAGTCATGGTAG | TGFBR2 target |
| | | sequence 56 |
| 175 | GACAACATCATCTTCTCAGA | TGFBR2 target |
| | | sequence 57 |
| 176 | TCCAGAATAAAGTCATGGTA | TGFBR2 target |
| | | sequence 58 |
| 177 | GGTAGGGGAGCTTGGGGTCA | TGFBR2 target |
| | | sequence 59 |
| 178 | TTCTCCAAAGTGCATTATGA | TGFBR2 target |
| | | sequence 60 |
| 179 | CATCTTCCAGAATAAAGTCA | TGFBR2 target |
| | | sequence 61 |
| 180 | CACATGAAGAAAGTCTCACC | TGFBR2 target |
| | | sequence 62 |
| 181 | TTCCAGAATAAAGTCATGGT | TGFBR2 target |
| | | sequence 63 |
| 182 | TTTTCCTTCATAATGCACTT | TGFBR2 target |
| | | sequence 64 |
| 183 | ASTKGPSVFPLAPCSRSTSESTAALGCLVKDYFPEPVTVSWNSGALTSGVHTFPAVLQSS | Human IgG2 Fc |
| | GLYSLSSVVTVPSSNFGTQTYTCNVDHKPSNTKVDKTVERKCCVECPPCPAPPVAGPSVF | (Uniprot |
| | LFPPKPKDTLMISRTPEVTCVVVDVSHEDPEVQFNWYVDGVEVHNAKTKPREEQFNSTFR | P01859) |
| | VVSVLTVVHQDWLNGKEYKCKVSNKGLPAPIEKTISKTKGQPREPQVYTLPPSREEMTKN | |
| | QVSLTCLVKGFYPSDISVEWESNGQPENNYKTTPPMLDSDGSFFLYSKLTVDKSRWQQGN | |
| | VFSCSVMHEALHNHYTQKSLSLSPGK | |
| 184 | ASTKGPSVFPLAPCSRSTSESTAALGCLVKDYFPEPVTVSWNSGALTSGVHTFPAVLQSS | Human IgG4 Fc |
| | GLYSLSSVVTVPSSSLGTKTYTCNVDHKPSNTKVDKRVESKYGPPCPSCPAPEFLGGPSV | (Uniprot |
| | FLFPPKPKDTLMISRTPEVTCVVVDVSQEDPEVQFNWYVDGVEVHNAKTKPREEQFNSTY | P01861) |
| | RVVSVLTVLHQDWLNGKEYKCKVSNKGLPSSIEKTISKAKGQPREPQVYTLPPSQEEMTK | |
| | NQVSLTCLVKGFYPSDIAVEWESNGQPENNYKTTPPVLDSDGSFFLYSRLTVDKSRWQEG | |
| | NVFSCSVMHEALHNHYTQKSLSLSLGK | |
| 185 | tgctttatttgtgaaatttgtgatgctattgctttatttgtaaccattataagctgcaat | SV40 poly A |
| | aaacaagttaacaacaacaattgcattcattttatgtttcaggttcagggggaggtgtgg | signal |
| | gaggttttttaaa | |
| 186 | gaacagagaaacaggagaatatgggccaaacaggatatctgtggtaagcagttcctgccc | MND promoter |
| | cggctcagggccaagaacagttggaacagcagaatatgggccaaacaggatatctgtggt | |
| | aagcagttcctgccccggctcagggccaagaacagatggtccccagatgcggtcccgccc | |
| | tcagcagtttctagagaaccatcagatgtttccagggtgccccaaggacctgaaatgacc | |
| | ctgtgccttatttgaactaaccaatcagttcgcttctcgcttctgttcgcgcgcttctgc | |
| | tccccgagctctatataagcagagctcgtttagtgaaccgtcagatc | |
| 187 | ESKYGPPCPPCPAPPVAGPSVFLFPPKPKDTLMISRTPEVTCVVVDVSQEDPEVQFNWYV | Spacer |
| | DGVEVHNAKTKPREEQFQSTYRVVSVLTVLHQDWLNGKEYKCKVSNKGLPSSIEKTISKA | |
| | KGQPREPQVYTLPPSQEEMTKNQVSLTCLVKGFYPSDIAVEWESNGQPENNYKTTPPVLD | |
| | SDGSFFLYSRLTVDKSRWQEGNVFSCSVMHEALHNHYTQKSLSLSLGK | |
| 188 | MKWKALFTAAILQAQLPITEAQSFGLLDPKLCYLLDGILFIYGVILTALFLRVKFSRSAD | CD3zeta |
| | APAYQQGQNQLYNELNLGRREEYDVLDKRRGRDPEMGGKPQRRKNPQEGLYNELQKDKMA | isoform 1 |
| | EAYSEIGMKGERRRGKGHDGLYQGLSTATKDTYDALHMQALPPR | precursor |
| | | protein |
| | | sequence |
| | | (NCBI |
| | | Reference |
| | | Sequence: |
| | | NP_932170.1) |
| 189 | MKWKALFTAAILQAQLPITEAQSFGLLDPKLCYLLDGILFIYGVILTALFLRVKFSRSAD | CD3 zeta |
| | APAYQQGQNQLYNELNLGRREEYDVLDKRRGRDPEMGGKPRRKNPQEGLYNELQKDKMAE | isoform 2 |
| | AYSEIGMKGERRRGKGHDGLYQGLSTATKDTYDALHMQALPPR | precursor |
| | | protein |
| | | sequence |
| | | (NCBI |
| | | Reference |
| | | Sequence: |
| | | NP_000725.1) |

1. A genetically engineered T cell, comprising a modified transforming growth factor-beta receptor type-2 (TGFBR2) locus, said modified TGFBR2 locus comprising a transgene sequence encoding a recombinant receptor or a portion thereof.

2. The genetically engineered T cell of claim 1, wherein the transgene sequence has been integrated at an endogenous TGFBR2 locus of a T cell, optionally via homology directed repair (HDR).

3. The genetically engineered T cell of claim 1 or 2, wherein the modified TGFBR2 locus:
does not encode a functional TGFBRII polypeptide;
does not encode a TGFBRII polypeptide or the expression of TGFBRII polypeptide is eliminated;
does not encode a full length TGFBRII polypeptide; and/or
encodes a dominant negative TGFBRII polypeptide, optionally wherein the dominant negative TGFBRII polypeptide comprises an amino acid sequence corresponding to residues 22-191 of SEQ ID NO:59 or residues 22-216 of SEQ ID NO:60, or a sequence that exhibits at least 85%, 86%, 87%, 88%, 89%, 90%, 91%, 92%, 93%, 94%, 95%, 96%, 97%, 98%, 99% or more sequence identity to an amino acid sequence corresponding to residues 22-191 of SEQ ID NO:59 or residues 22-216 of SEQ ID NO:60, or a fragment thereof.

4. The genetically engineered T cell of any of claims 1-3, wherein the transgene sequence is in-frame with one or more exons of an open reading frame or partial sequence thereof, of the endogenous TGFBR2.

5. The genetically engineered T cell of any of claims 1-4, wherein the transgene sequence is downstream of exon 1 and upstream of exon 6, of the open reading frame of the endogenous TGFBR2 locus.

6. The genetically engineered T cell of any of claims 1-5, wherein the transgene sequence is downstream of exon 4 and upstream of exon 6, of the open reading frame of the endogenous TGFBR2 locus.

7. The genetically engineered T cell of any of claims 1-6, wherein the recombinant receptor is or comprises a recombinant T cell receptor (TCR), and the transgene sequence encodes a TCR alpha (TCRα) chain, a TCR beta (TCRβ) chain or both.

8. The genetically engineered T cell of any of claims 1-6, wherein the recombinant receptor is a chimeric antigen receptor (CAR), wherein the CAR comprises an extracellular region comprising a binding domain, a transmembrane domain, and an intracellular region.

9. The genetically engineered T cell of claim 8, wherein the binding domain is or comprises an antibody or an antigen-binding fragment thereof.

10. The genetically engineered T cell of claim 8 or 9, wherein the binding domain is capable of binding to a target antigen that is associated with, specific to, or expressed on a cell or tissue of a disease, disorder or condition, optionally wherein the target antigen is a tumor antigen.

11. The genetically engineered T cell of claim 10, wherein the target antigen is selected from among αvβ6 integrin (avb6 integrin), B cell maturation antigen (BCMA), B7-H3, B7-H6, carbonic anhydrase 9 (CA9, also known as CAIX or G250), a cancer-testis antigen, cancer/testis antigen 1B (CTAG, also known as NY-ESO-1 and LAGE-2), carcinoembryonic antigen (CEA), a cyclin, cyclin A2, C—C Motif Chemokine Ligand 1 (CCL-1), CD19, CD20, CD22, CD23, CD24, CD30, CD33, CD38, CD44, CD44v6, CD44v7/8, CD123, CD133, CD138, CD171, chondroitin sulfate proteoglycan 4 (CSPG4), epidermal growth factor protein (EGFR), type III epidermal growth factor receptor mutation (EGFR vIII), epithelial glycoprotein 2 (EPG-2), epithelial glycoprotein 40 (EPG-40), ephrinB2, ephrin receptor A2 (EPHa2), estrogen receptor, Fc receptor like 5 (FCRL5; also known as Fc receptor homolog 5 or FCRH5), fetal acetylcholine receptor (fetal AchR), a folate binding protein (FBP), folate receptor alpha, ganglioside GD2, O-acetylated GD2 (OGD2), ganglioside GD3, glycoprotein 100 (gp100), glypican-3 (GPC3), G protein-coupled receptor class C group 5 member D (GPRC5D), Her2/neu (receptor tyrosine kinase erb-B2), Her3 (erb-B3), Her4 (erb-B4), erbB dimers, Human high molecular weight-melanoma-associated antigen (HMW-MAA), hepatitis B surface antigen, Human leukocyte antigen A1 (HLA-A1), Human leukocyte antigen A2 (HLA-A2), IL-22 receptor alpha (IL-22Rα), IL-13 receptor alpha 2 (IL-13Rα2), kinase insert domain receptor (kdr), kappa light chain, L1 cell adhesion molecule (L1-CAM), CE7 epitope of L1-CAM, Leucine Rich Repeat Containing 8 Family Member A (LRRC8A), Lewis Y, Melanoma-associated antigen (MAGE)-A1, MAGE-A3, MAGE-A6, MAGE-A10, mesothelin (MSLN), c-Met, murine cytomegalovirus (CMV), mucin 1 (MUC1), MUC16, natural killer group 2 member D (NKG2D) ligands, melan A (MART-1), neural cell adhesion molecule (NCAM), oncofetal antigen, Preferentially expressed antigen of melanoma (PRAME), progesterone receptor, a prostate specific antigen, prostate stem cell antigen (PSCA), prostate specific membrane antigen (PSMA), Receptor Tyrosine Kinase Like Orphan Receptor 1 (ROR1), survivin, Trophoblast glycoprotein (TPBG also known as 5T4), tumor-associated glycoprotein 72 (TAG72), Tyrosinase related protein 1 (TRP1, also known as TYRP1 or gp75), Tyrosinase related protein 2 (TRP2, also known as dopachrome tautomerase, dopachrome delta-isomerase or DCT), vascular endothelial growth factor receptor (VEGFR), vascular endothelial growth factor receptor 2 (VEGFR2), Wilms Tumor 1 (WT-1), a pathogen-specific or pathogen-expressed antigen, or an antigen associated with a universal tag, and/or biotinylated molecules, and/or molecules expressed by HIV, HCV, HBV or other pathogens.

12. The genetically engineered T cell of any of claims 8-11, wherein the extracellular region comprises a spacer, optionally wherein the spacer is operably linked between the binding domain and the transmembrane domain.

13. The genetically engineered T cell of claim 12, wherein the spacer comprises an immunoglobulin hinge region and/or a C_{H}2 region and a C_{H}3 region.

14. The genetically engineered T cell of any of claims 8-13, wherein the intracellular region comprises an intracellular signaling domain.

15. The genetically engineered T cell of claim 14, wherein the intracellular signaling domain is or comprises an intracellular signaling domain of a CD3 chain, optionally a CD3-zeta (CD3) chain, or a signaling portion thereof.

16. The genetically engineered T cell of any of claims 8-15, wherein the intracellular region comprises one or more costimulatory signaling domain(s).

17. The genetically engineered T cell of claim 16, wherein the one or more costimulatory signaling domain comprises an intracellular signaling domain of a CD28, a 4-1BB or an ICOS or a signaling portion thereof.

18. The genetically engineered T cell of any of claims 1-17, wherein
the transgene sequence comprises, in order: a sequence of nucleotides encoding a binding domain, optionally a single chain Fv fragment (scFv); a spacer, optionally comprising a sequence from a human immunoglobulin hinge, optionally from IgG1, IgG2 or IgG4 or a modified version thereof, optionally further comprising a C_{H}2 region and/or a C_{H}3 region; and a transmembrane domain, optionally from human CD28; a costimulatory signaling domain, optionally from human 4-1BB; and an intracellular signaling region, optionally a CD3ζ chain or a portion thereof; and/or
the modified TGFBR2 locus comprises, in order: a sequence of nucleotides encoding a binding domain, optionally an scFv; a spacer, optionally comprising a sequence from a human immunoglobulin hinge, optionally from IgG1, IgG2 or IgG4 or a modified version thereof, optionally further comprising a C_{H}2 region and/or a C_{H}3 region; and a transmembrane domain, optionally from human CD28; a costimulatory signaling domain, optionally from human 4-1BB; and an intracellular signaling region, optionally a CD3ζ chain or a portion thereof.

19. The genetically engineered T cell of any of claims 1-18, wherein the transgene sequence comprises a sequence of nucleotides encoding at least one further protein.

20. The genetically engineered T cell of claim 19, wherein the at least one further protein is a surrogate marker, optionally wherein the surrogate marker is a truncated receptor, optionally wherein the truncated receptor lacks an intracellular signaling domain and/or is not capable of mediating intracellular signaling when bound by its ligand.

21. The genetically engineered T cell of any of claims 1-20, wherein the transgene sequence comprises one or more multicistronic element(s).

22. The genetically engineered T cell of claim 21, wherein:
the transgene sequence comprises a sequence of nucleotides encoding the recombinant receptor or a portion thereof, and the one or more multicistronic element(s) are positioned upstream of the sequence of nucleotides encoding the recombinant receptor or a portion thereof; and/or positioned between the sequence of nucleotides encoding the recombinant receptor or a portion thereof and the sequence of nucleotides encoding the at least one further protein; and/or
the recombinant receptor is a TCR, and the one or more multicistronic element(s) are positioned between a sequence of nucleotides encoding the TCRα and a sequence of nucleotides encoding the TCRβ; and/or
the recombinant receptor is a CAR that a multi-chain CAR, and the one or more multicistronic element(s) are positioned between a sequence of nucleotides encoding one chain of the multi-chain CAR and a sequence of nucleotides encoding another chain of the multi-chain CAR.

23. The genetically engineered T cell of claim 21 or 22, wherein the one or more multicistronic element is or comprises a ribosome skip sequence, optionally wherein the ribosome skip sequence is a T2A, a P2A, an E2A, or an F2A element.

24. The genetically engineered T cell of any of claims 1-23, wherein the modified TGFBR2 locus comprises the promoter and/or regulatory or control element of the endogenous TGFBR2 locus operably linked to control expression the transgene sequence encoding the recombinant receptor or a portion thereof; or the modified TGFBR2 locus comprises one or more heterologous regulatory or control element(s) operably linked to control expression of the recombinant receptor or a portion thereof.

25. The genetically engineered T cell of any of claims 1-24, wherein the T cell is a primary T cell derived from a subject, optionally wherein the subject is a human.

26. The genetically engineered T cell of any of claims 1-25, wherein the T cell is a CD8+ T cell or subtypes thereof or a CD4+ T cell or subtypes thereof.

27. A polynucleotide, comprising:
(a) a nucleic acid sequence encoding a recombinant receptor or a portion thereof; and
(b) one or more homology arm(s) linked to the nucleic acid sequence, wherein the one or more homology arm(s) comprise a sequence homologous to one or more region(s) of an open reading frame of transforming growth factor-beta receptor type-2 (TGFBR2) locus.

28. The polynucleotide of claim 27, wherein the nucleic acid sequence of (a) is a sequence that is exogenous or heterologous to the open reading frame of the endogenous TGFBR2 locus of a T cell, optionally a human T cell.

29. The polynucleotide of claim 27 or 28, wherein the one or more homology arm(s) comprise at least one intron or at least one exon of the open reading frame of the TGFBR2 locus of a T cell, optionally a human T cell.

30. The polynucleotide of any of claims 27-29, wherein the nucleic acid sequence of (a) is in-frame with one or more exons of the open reading frame of the TGFBR2 locus comprised in the one or more homology arm(s).

31. The polynucleotide of any of claims 27-30, wherein the one or more region(s) of the open reading frame is or comprises sequences that are downstream of exon 1 of the open reading frame of the TGFBR2 locus.

32. The polynucleotide of any of claims 27-31, wherein the one or more region(s) of the open reading frame is or comprises sequences that includes at least a portion of exon 4 or downstream of exon 4 of the open reading frame of the TGFBR2 locus.

33. The polynucleotide of any of claims 27-32, wherein the one or more homology arm comprises a 5′ homology arm and a 3′ homology arm, and the polynucleotide comprises the structure [5′ homology arm]-[nucleic acid sequence of (a)]-[3′ homology arm].

34. The polynucleotide of claim 33, wherein the 5′ homology arm and the 3′ homology arm independently are at or about 200, 300, 400, 500, 600, 700 or 800 nucleotides in length, or any value between any of the foregoing, or are greater than at or about 300 nucleotides in length, optionally at or about 400, 500 or 600 nucleotides in length, or any value between any of the foregoing.

35. The polynucleotide of claim 33 or 34, wherein the 5′ homology arm comprises the sequence set forth in SEQ ID NOS: 69-71 or a sequence that exhibits at least 85%, 86%, 87%, 88%, 89%, 90%, 91%, 92%, 93%, 94%, 95%, 96%, 97%, 98%, 99% or more sequence identity to SEQ ID NOS: 69-71 or a partial sequence thereof, and/or the 3′ homology arm comprises the sequence set forth in SEQ ID NO:72, or a sequence that exhibits at least 85%, 86%, 87%, 88%, 89%, 90%, 91%, 92%, 93%, 94%, 95%, 96%, 97%, 98%, 99% or more sequence identity to SEQ ID NO:72 or a partial sequence thereof.

36. The polynucleotide of any of claims 27-35 wherein the encoded recombinant receptor is or comprises a recombinant T cell receptor (TCR), and the nucleic acid sequence of (a) encodes a TCR alpha (TCRα) chain, a TCR beta (TCRβ) chain or both.

37. The polynucleotide of any of claims 27-35, wherein the encoded recombinant receptor is a chimeric antigen receptor (CAR), wherein the CAR comprises an extracellular region comprising a binding domain, a transmembrane domain, and an intracellular region.

38. The polynucleotide of claim 37, wherein the binding domain is or comprises an antibody or an antigen-binding fragment thereof.

39. The polynucleotide of claim 37 or 38, wherein the binding domain is capable of binding to a target antigen that is associated with, specific to, or expressed on a cell or tissue of a disease, disorder or condition, optionally wherein the target antigen is a tumor antigen.

40. The polynucleotide of claim 39, wherein the target antigen is selected from among αvβ6 integrin (avb6 integrin), B cell maturation antigen (BCMA), B7-H3, B7-H6, carbonic anhydrase 9 (CA9, also known as CAIX or G250), a cancer-testis antigen, cancer/testis antigen 1B (CTAG, also known as NY-ESO-1 and LAGE-2), carcinoembryonic antigen (CEA), a cyclin, cyclin A2, C—C Motif Chemokine Ligand 1 (CCL-1), CD19, CD20, CD22, CD23, CD24, CD30, CD33, CD38, CD44, CD44v6, CD44v7/8, CD123, CD133, CD138, CD171, chondroitin sulfate proteoglycan 4 (CSPG4), epidermal growth factor protein (EGFR), type III epidermal growth factor receptor mutation (EGFR vIII), epithelial glycoprotein 2 (EPG-2), epithelial glycoprotein 40 (EPG-40), ephrinB2, ephrin receptor A2 (EPHa2), estrogen receptor, Fc receptor like 5 (FCRL5; also known as Fc receptor homolog 5 or FCRH5), fetal acetylcholine receptor (fetal AchR), a folate binding protein (FBP), folate receptor alpha, ganglioside GD2, O-acetylated GD2 (OGD2), ganglioside GD3, glycoprotein 100 (gp100), glypican-3 (GPC3), G protein-coupled receptor class C group 5 member D (GPRC5D), Her2/neu (receptor tyrosine kinase erb-B2), Her3 (erb-B3), Her4 (erb-B4), erbB dimers, Human high molecular weight-melanoma-associated antigen (HMW-MAA), hepatitis B surface antigen, Human leukocyte antigen A1 (HLA-A1), Human leukocyte antigen A2 (HLA-A2), IL-22 receptor alpha (IL-22Rα), IL-13 receptor alpha 2 (IL-13Rα2), kinase insert domain receptor (kdr), kappa light chain, L1 cell adhesion molecule (L1-CAM), CE7 epitope of L1-CAM, Leucine Rich Repeat Containing 8 Family Member A (LRRC8A), Lewis Y, Melanoma-associated antigen (MAGE)-A1, MAGE-A3, MAGE-A6, MAGE-A10, mesothelin (MSLN), c-Met, murine cytomegalovirus (CMV), mucin 1 (MUC1), MUC16, natural killer group 2 member D (NKG2D) ligands, melan A (MART-1), neural cell adhesion molecule (NCAM), oncofetal antigen, Preferentially expressed antigen of melanoma (PRAME), progesterone receptor, a prostate specific antigen, prostate stem cell antigen (PSCA), prostate specific membrane antigen (PSMA), Receptor Tyrosine Kinase Like Orphan Receptor 1 (ROR1), survivin, Trophoblast glycoprotein (TPBG also known as 5T4), tumor-associated glycoprotein 72 (TAG72), Tyrosinase related protein 1 (TRP1, also known as TYRP1 or gp75), Tyrosinase related protein 2 (TRP2, also known as dopachrome tautomerase, dopachrome delta-isomerase or DCT), vascular endothelial growth factor receptor (VEGFR), vascular endothelial growth factor receptor 2 (VEGFR2), Wilms Tumor 1 (WT-1), a pathogen-specific or pathogen-expressed antigen, or an antigen associated with a universal tag, and/or biotinylated molecules, and/or molecules expressed by HIV, HCV, HBV or other pathogens.

41. The polynucleotide of any of claims 37-40, wherein the extracellular region comprises a spacer, optionally wherein the spacer is operably linked between the binding domain and the transmembrane domain.

42. The polynucleotide of claim 41, wherein the spacer comprises an immunoglobulin hinge region and/or a C_{H}2 region and a C_{H}3 region.

43. The polynucleotide of any of claims 37-42, wherein the intracellular region comprises an intracellular signaling domain.

44. The polynucleotide of claim 43, wherein the intracellular signaling domain is or comprises an intracellular signaling domain of a CD3 chain, optionally a CD3-zeta (CD3) chain, or a signaling portion thereof.

45. The polynucleotide of any of claims 37-44, wherein the intracellular region comprises one or more costimulatory signaling domain(s).

46. The polynucleotide of claim 45, wherein the one or more costimulatory signaling domain comprises an intracellular signaling domain of a CD28, a 4-1BB or an ICOS or a signaling portion thereof.

47. The polynucleotide of any of claims 27-46, wherein the nucleic acid sequence of (a) comprises, in order: a sequence of nucleotides encoding a binding domain, optionally a single chain Fv fragment (scFv); a spacer, optionally comprising a sequence from a human immunoglobulin hinge, optionally from IgG1, IgG2 or IgG4 or a modified version thereof, optionally further comprising a C_{H}2 region and/or a C_{H}3 region; and a transmembrane domain, optionally from human CD28; a costimulatory signaling domain, optionally from human 4-1BB; and an intracellular signaling region, optionally a CD3ζ chain or a portion thereof.

48. The polynucleotide of any of claims 27-47, wherein the nucleic acid sequence of (a) comprises a sequence of nucleotides encoding at least one further protein.

49. The polynucleotide of claim 48, wherein the at least one further protein is a surrogate marker, optionally wherein the surrogate marker is a truncated receptor, optionally wherein the truncated receptor lacks an intracellular signaling domain and/or is not capable of mediating intracellular signaling when bound by its ligand.

50. The polynucleotide of any of claims 27-49, wherein the nucleic acid sequence of (a) comprises one or more multicistronic element(s).

51. The polynucleotide of claim 50, wherein:
the nucleic acid sequence of (a) comprises a sequence of nucleotides encoding the recombinant receptor or a portion thereof, and the one or more multicistronic element(s) are positioned upstream of the sequence of nucleotides encoding the recombinant receptor or a portion thereof; and/or positioned between the sequence of nucleotides encoding the recombinant receptor or a portion thereof and the sequence of nucleotides encoding the at least one further protein; and/or
the recombinant receptor is a TCR, and the one or more multicistronic element(s) are positioned between a sequence of nucleotides encoding the TCRα and a sequence of nucleotides encoding the TCRβ; and/or
the recombinant receptor is a CAR that a multi-chain CAR, and the one or more multicistronic element(s) are positioned between a sequence of nucleotides encoding one chain of the multi-chain CAR and a sequence of nucleotides encoding another chain of the multi-chain CAR.

52. The polynucleotide of claim 50 or 51, wherein the one or more multicistronic element is or comprises a ribosome skip sequence, optionally wherein the ribosome skip sequence is a T2A, a P2A, an E2A, or an F2A element.

53. The polynucleotide of any of claims 27-52, wherein the nucleic acid sequence of (a) comprises one or more heterologous regulatory or control element(s) operably linked to control expression of the recombinant receptor or a portion thereof.

54. The polynucleotide of any of claims 27-53, wherein the polynucleotide is comprised in a viral vector.

55. The polynucleotide of claim 54, wherein the viral vector is an AAV vector, optionally wherein the AAV vector is an AAV2 or AAV6 vector.

56. The polynucleotide of claim 54, wherein the viral vector is a retroviral vector, optionally a lentiviral vector.

57. The polynucleotide of any of claims 27-53, that is a linear polynucleotide, optionally a double-stranded polynucleotide or a single-stranded polynucleotide.

58. The polynucleotide of any of claims 27-57, wherein the polynucleotide is between at or about 2500 and at or about 5000 nucleotides, at or about 3500 and at or about 4500 nucleotides, or at or about 3750 nucleotides and at or about 4250 nucleotides in length.

59. A method of producing a genetically engineered T cell, the method comprising introducing the polynucleotide of any of claims 27-58 into a T cell comprising a genetic disruption at a TGFBR2 locus.

60. A method of producing a genetically engineered T cell, the method comprising:
(a) introducing, into a T cell, one or more agent(s) capable of inducing a genetic disruption at a target site within an endogenous TGFBR2 locus of the T cell; and
(b) introducing the polynucleotide of any of claims 27-58 into a T cell comprising a genetic disruption at a TGFBR2 locus.

61. The method of claim 59 or 60, wherein the nucleic acid sequence encoding a recombinant receptor or a portion thereof is integrated within the endogenous TGFBR2 locus via homology directed repair (HDR).

62. A method of producing a genetically engineered T cell, the method comprising introducing, into a T cell, a polynucleotide comprising a nucleic acid sequence encoding a recombinant receptor or a portion thereof, said T cell having a genetic disruption within a TGFBR2 locus of the T cell, wherein the nucleic acid sequence encoding the recombinant receptor or a portion thereof is integrated within the endogenous TGFBR2 locus via homology directed repair (HDR).

63. The method of any of claims 59, 61 and 62, wherein the genetic disruption is carried out by introducing, into a T cell, one or more agent(s) capable of inducing a genetic disruption at a target site within an endogenous TGFBR2 locus of the T cell.

64. The method of any of claims 59-63, wherein the method produces a modified TGFBR2 locus in the T cell, said modified TGFBR2 locus comprising a nucleic acid sequence encoding a recombinant receptor or a portion thereof.

65. The method of any of claims 62-64, wherein the polynucleotide further comprises one or more homology arm(s) linked to the nucleic acid sequence, wherein the one or more homology arm(s) comprise a sequence homologous to one or more region(s) of an open reading frame of a transforming growth factor-beta receptor type-2 (TGFBR2) locus.

66. The method of any of claims 59-65, wherein, in a cell generated by the method, the modified TGFBR2 locus:
does not encode a functional TGFBRII polypeptide, in a cell generated by the method;
does not encode a TGFBRII polypeptide or the expression of TGFBRII polypeptide is eliminated; and/or
does not encode a full length TGFBRII polypeptide or encodes a dominant negative TGFBRII polypeptide.

67. The method of claim 65 or 66, wherein the one or more homology arm comprises a 5′ homology arm and a 3′ homology arm, and the polynucleotide comprises the structure [5′ homology arm]-[the nucleic acid sequence encoding a recombinant receptor or a portion thereof]-[3′ homology arm].

68. The method of any of claims 59-67, wherein the encoded recombinant receptor is or comprises recombinant T cell receptor (TCR).

69. The method of any of claims 59-67, wherein the encoded recombinant receptor is a chimeric antigen receptor (CAR).

70. The method of any of claims 60 and 63-69, wherein the one or more agent(s) capable of inducing a genetic disruption comprises a DNA binding protein or DNA-binding nucleic acid that specifically binds to or hybridizes to the target site, a fusion protein comprising a DNA-targeting protein and a nuclease, or an RNA-guided nuclease, optionally wherein the one or more agent(s) comprises a zinc finger nuclease (ZFN), a TAL-effector nuclease (TALEN), or and a CRISPR-Cas9 combination that specifically binds to, recognizes, or hybridizes to the target site.

71. The method of any of claims 60 and 63-70, wherein the one or more agent(s) comprises a guide RNA (gRNA) having a targeting domain that is complementary to the at least one target site.

72. The method of claim 71, wherein the one or more agent(s) is introduced as a ribonucleoprotein (RNP) complex comprising the gRNA and a Cas9 protein, optionally wherein the RNP is introduced via electroporation, particle gun, calcium phosphate transfection, cell compression or squeezing, optionally via electroporation.

73. The method of claim 72, wherein the concentration of the RNP is from at or about 1 μM to at or about 5 μM, optionally wherein the concentration of the RNP is at or about 2 μM.

74. The method of any of claims 71-73, wherein the gRNA has a targeting domain sequence of GUGGAUGACCUGGCUAACAG (SEQ ID NO:73).

75. The method of any of claims 59-74, wherein the T cell is a primary T cell derived from a subject, optionally wherein the subject is a human.

76. The method of any of claims 59-75, wherein the T cell is a CD8+ T cell or subtypes thereof, or a CD4+ T cell or subtypes thereof.

77. The method of any of claims 59-76, wherein the polynucleotide is comprised in a viral vector.

78. The method of claim 77, wherein the viral vector is an AAV vector, optionally wherein the AAV vector is an AAV2 or AAV6 vector.

79. The method of any of claims 59-78, wherein the polynucleotide is a linear polynucleotide, optionally a double-stranded polynucleotide or a single-stranded polynucleotide.

80. The method of any of claims 60 and 63-79, wherein the polynucleotide is introduced after the introduction of the one or more agent(s).

81. The method of claim 80, wherein the polynucleotide is introduced immediately after, or within about 30 seconds, 1 minute, 2 minutes, 3 minutes, 4 minutes, 5 minutes, 6 minutes, 6 minutes, 8 minutes, 9 minutes, 10 minutes, 15 minutes, 20 minutes, 30 minutes, 40 minutes, 50 minutes, 60 minutes, 90 minutes, 2 hours, 3 hours or 4 hours after the introduction of the agent.

82. The method of any of claims 60 and 64-81, wherein prior to the introducing of the one or more agent, the method comprises incubating the cells, in vitro with one or more stimulatory agent(s) under conditions to stimulate or activate the one or more immune cells, optionally wherein the one or more stimulatory agent(s) comprises and anti-CD3 and/or anti-CD28 antibodies, optionally anti-CD3/anti-CD28 beads, optionally wherein the bead to cell ratio is or is about 1:1.

83. The method of any of claims 60 and 64-82, wherein the method further comprises incubating the cells prior to, during or subsequent to the introducing of the one or more agents and/or the introducing of the polynucleotide with one or more recombinant cytokines, optionally wherein the one or more recombinant cytokines are selected from the group consisting of IL-2, IL-7, and IL-15, optionally wherein the one or more recombinant cytokine is added at a concentration selected from a concentration of IL-2 from at or about 10 U/mL to at or about 200 U/mL, optionally at or about 50 IU/mL to at or about 100 U/mL; IL-7 at a concentration of 0.5 ng/mL to 50 ng/mL, optionally at or about 5 ng/mL to at or about 10 ng/mL and/or IL-15 at a concentration of 0.1 ng/mL to 20 ng/mL, optionally at or about 0.5 ng/mL to at or about 5 ng/mL.

84. The method of claim 82 or 83, wherein the incubation is carried out subsequent to the introducing of the one or more agents and the introducing of the polynucleotide for up to or approximately 24 hours, 36 hours, 48 hours, 3, 4, 5, 6, 7, 8, 9, 10, 11, 12, 13, 14, 15, 16, 17, 18, 19, 20 or 21 days, optionally up to or about 7 days.

85. The method of any of claims 59-84, wherein at least or greater than 35%, 40%, 45%, 50%, 55%, 60%, 65%, 70%, 75%, 80%, or 90% of the cells in a plurality of engineered cells generated by the method comprise a genetic disruption of at least one target site within a TGFBR2 locus; and/or at least or greater than 35%, 40%, 45%, 50%, 55%, 60%, 65%, 70%, 75%, 80%, or 90% of the cells in a plurality of engineered cells generated by the method express the recombinant receptor.

86. A genetically engineered T cell or a plurality of genetically engineered T cells generated using the method of any of claims 59-85.

87. A composition, comprising the genetically engineered T cell any of claims 1-26 and 86;
or a plurality of the genetically engineered T cell of any of claims 1-26 and 86.

88. The composition of claim 87, wherein the composition comprises CD4+ T cells and/or CD8+ T cells.

89. The composition of claim 88, wherein the composition comprises CD4+ T cells and CD8+ T cells and the ratio of CD4+ to CD8+ T cells is from or from about 1:3 to 3:1, optionally 1:1.

90. The composition of any of claims 87-89, wherein cells expressing the recombinant receptor make up at least 30%, 40%, 50%, 60%, 70%, 80%, 90%, 91%, 92%, 93%, 94%, 95%, 96%, 97%, 98%, 99%, or more of the total cells in the composition or of the total CD4+ T cells or CD8+ T cells in the composition.

91. A method of treatment comprising administering the genetically engineered T cell, plurality of genetically engineered T cells or composition of any of claims 1-26 and 86-90 to a subject having a disease or disorder.

92. Use of the genetically engineered T cell, plurality of genetically engineered T cells or composition of any of claims 1-26 and 86-90 for the treatment of a disease or disorder.

93. Use of the genetically engineered T cell, plurality of genetically engineered T cells or composition of any of claims 1-26 and 86-90 in the manufacture of a medicament for treating a disease or disorder.

94. The genetically engineered T cell, plurality of genetically engineered T cells or composition of any of claims 1-26 and 86-90 for use in the treatment of a disease or disorder.

95. The method, use or the genetically engineered T cell, plurality of genetically engineered T cells or composition for use of any of claims 91-94, wherein the disease or disorder is a cancer or a tumor.

96. The method, use or the genetically engineered T cell, plurality of genetically engineered T cells or composition for use of claim 95, wherein the cancer or the tumor is a hematologic malignancy, optionally a lymphoma, a leukemia, or a plasma cell malignancy.

97. The method, use or the genetically engineered T cell, plurality of genetically engineered T cells or composition for use of claim 95, wherein the cancer or the tumor is a solid tumor, optionally wherein the solid tumor is a non-small cell lung cancer (NSCLC) or a head and neck squamous cell carcinoma (HNSCC).

98. A kit comprising:
one or more agent(s) capable of inducing a genetic disruption at a target site within a TGFBR2 locus; and
the polynucleotide of any of claims 27-58.

99. A kit, comprising:
one or more agent(s) capable of inducing a genetic disruption at a target site within a TGFBR2 locus; and
a polynucleotide comprising a nucleic acid sequence encoding a recombinant receptor or a portion thereof, wherein the nucleic acid sequence encoding the recombinant receptor or a portion thereof is targeted for integration at or near the target site via homology directed repair (HDR); and
instructions for carrying out the method of any of claims 59-85.


SEQUENCE LISTING (189 sequences)

SEQ ID NO: 1 - protein, 12 aa, Homo sapiens
  spacer (IgG4hinge)
ESKYGPPCPPCP

SEQ ID NO: 2 - DNA, 36 bp, Homo sapiens
  spacer (IgG4hinge)
gaatctaagtacggaccgccctgccccccttgccct

SEQ ID NO: 3 - protein, 119 aa, Homo sapiens
  Hinge-CH3 spacer
ESKYGPPCPPCPGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 4 - protein, 229 aa, Homo sapiens
  Hinge-CH2-CH3 spacer
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 5 - protein, 282 aa, Artificial Sequence
  IgD-hinge-Fc
RWPESPKAQASSVPTAQPQAEGSLAKATTAPATTRNTGRGGEEKKKEKEKEEQEERETKTPECPSHTQPLGVYLLTPAVQDLWLRDKATFTCFVVGSDLKDAHLTWEVAGKVPTGGVEEGLLERHSNGSQSQHSRLTLPRSLWNAGTSVTCTLNHPSLPPQRLMALREPAAQAPVKLSLNLLASSDPPEAASWLLCEVSGFSPPNILLMWLEDQREVNTSGFAPARPPPQPGSTTFWAWSVLRVPAPPSPQPATYTCVVSHEDSRTLLNASRSLEVSYVTDH

SEQ ID NO: 6 - protein, 24 aa, Artificial Sequence
  T2A
LEGGGEGRGSLLTCGDVEENPGPR

SEQ ID NO: 7 - protein, 357 aa, Artificial Sequence
  tEGFR
MLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 8 - protein, 27 aa, Homo sapiens
  CD28
FWVLVVVGGVLACYSLLVTVAFIIFWV

SEQ ID NO: 9 - protein, 66 aa, Homo sapiens
  CD28
IEVMYPPPYLDNEKSNGTIIHVKGKHLCPSPLFPGPSKPFWVLVVVGGVLACYSLLVTVAFIIFWV

SEQ ID NO: 10 - protein, 41 aa, Homo sapiens
  CD28
RSKRSRLLHSDYMNMTPRRPGPTRKHYQPYAPPRDFAAYRS

SEQ ID NO: 11 - protein, 41 aa, Homo sapiens
  CD28 (LL to GG)
RSKRSRGGHSDYMNMTPRRPGPTRKHYQPYAPPRDFAAYRS

SEQ ID NO: 12 - protein, 42 aa, Homo sapiens
  4-1BB
KRGRKKLLYIFKQPFMRPVQTTQEEDGCSCRFPEEEEGGCEL

SEQ ID NO: 13 - protein, 112 aa, Homo sapiens
  CD3 zeta
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 14 - protein, 112 aa, Homo sapiens
  CD3 zeta
RVKFSRSAEPPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 15 - protein, 112 aa, Homo sapiens
  CD3 zeta
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 16 - protein, 335 aa, Artificial Sequence
  tEGFR
RKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 17 - protein, 18 aa, Artificial Sequence
  T2A
EGRGSLLTCGDVEENPGP

SEQ ID NO: 18 - protein, 22 aa, Artificial Sequence
  P2A
GSGATNFSLLKQAGDVEENPGP

SEQ ID NO: 19 - protein, 19 aa, Artificial Sequence
  P2A
ATNFSLLKQAGDVEENPGP

SEQ ID NO: 20 - protein, 20 aa, Artificial Sequence
  E2A
QCTNYALLKLAGDVESNPGP

SEQ ID NO: 21 - protein, 22 aa, Artificial Sequence
  F2A
VKQTLNFDLLKLAGDVESNPGP

SEQ ID NO: 22 - protein, 10 aa, Artificial Sequence
  Linker
  REPEAT (5)...(9): SGGGG is repeated 5 times
PGGGSGGGGP

SEQ ID NO: 23 - protein, 17 aa, Artificial Sequence
  Linker
GSADDAKKDAAKKDGKS

SEQ ID NO: 24 - DNA, 66 bp, Artificial Sequence
  GMCSFR alpha chain signal sequence
atgcttctcctggtgacaagccttctgctctgtgagttaccacacccagcattcctcctgatccca

SEQ ID NO: 25 - protein, 22 aa, Artificial Sequence
  GMCSFR alpha chain signal sequence
MLLLVTSLLLCELPHPAFLLIP

SEQ ID NO: 26 - protein, 18 aa, Artificial Sequence
  CD8 alpha signal peptide
MALPVTALLLPLALLLHA

SEQ ID NO: 27 - protein, 15 aa, Artificial Sequence
  Hinge
EPKSCDKTHTCPPCP

SEQ ID NO: 28 - protein, 12 aa, Artificial Sequence
  Hinge
ERKCCVECPPCP

SEQ ID NO: 29 - protein, 61 aa, Artificial Sequence
  Hinge
ELKTPLGDTHTCPRCPEPKSCDTPPPCPRCPEPKSCDTPPPCPRCPEPKSCDTPPPCPRCP

SEQ ID NO: 30 - protein, 12 aa, Artificial Sequence
  Hinge
ESKYGPPCPSCP

SEQ ID NO: 31 - protein, 5 aa, Artificial Sequence
  Hinge
  VARIANT (1)...(1): Xaa is glycine, cysteine or arginine
  VARIANT (4)...(4): Xaa is cysteine or threonine
XPPXP

SEQ ID NO: 32 - protein, 9 aa, Artificial Sequence
  Hinge
YGPPCPPCP

SEQ ID NO: 33 - protein, 10 aa, Artificial Sequence
  Hinge
KYGPPCPPCP

SEQ ID NO: 34 - protein, 14 aa, Artificial Sequence
  Hinge
EVVVKYGPPCPPCP

SEQ ID NO: 35 - protein, 11 aa, Artificial Sequence
  CDR L1
RASQDISKYLN

SEQ ID NO: 36 - protein, 7 aa, Artificial Sequence
  CDR L2
SRLHSGV

SEQ ID NO: 37 - protein, 9 aa, Artificial Sequence
  CDR L3
GNTLPYTFG

SEQ ID NO: 38 - protein, 5 aa, Artificial Sequence
  CDR H1
DYGVS

SEQ ID NO: 39 - protein, 16 aa, Artificial Sequence
  CDR H2
VIWGSETTYYNSALKS

SEQ ID NO: 40 - protein, 7 aa, Artificial Sequence
  CDR H3
YAMDYWG

SEQ ID NO: 41 - protein, 120 aa, Artificial Sequence
  VH
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 42 - protein, 107 aa, Artificial Sequence
  VL
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIT

SEQ ID NO: 43 - protein, 245 aa, Artificial Sequence
  scFv
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 44 - protein, 11 aa, Artificial Sequence
  CDR L1
KASQNVGTNVA

SEQ ID NO: 45 - protein, 7 aa, Artificial Sequence
  CDR L2
SATYRNS

SEQ ID NO: 46 - protein, 9 aa, Artificial Sequence
  CDR L3
QQYNRYPYT

SEQ ID NO: 47 - protein, 5 aa, Artificial Sequence
  CDR H1
SYWMN

SEQ ID NO: 48 - protein, 17 aa, Artificial Sequence
  CDR H2
QIYPGDGDTNYNGKFKG

SEQ ID NO: 49 - protein, 13 aa, Artificial Sequence
  CDR H3
KTISSVVDFYFDY

SEQ ID NO: 50 - protein, 122 aa, Artificial Sequence
  VH
EVKLQQSGAELVRPGSSVKISCKASGYAFSSYWMNWVKQRPGQGLEWIGQIYPGDGDTNYNGKFKGQATLTADKSSSTAYMQLSGLTSEDSAVYFCARKTISSVVDFYFDYWGQGTTVTVSS

SEQ ID NO: 51 - protein, 108 aa, Artificial Sequence
  VL
DIELTQSPKFMSTSVGDRVSVTCKASQNVGTNVAWYQQKPGQSPKPLIYSATYRNSGVPDRFTGSGSGTDFTLTITNVQSKDLADYFCQQYNRYPYTSGGGTKLEIKR

SEQ ID NO: 52 - protein, 15 aa, Artificial Sequence
  Linker
GGGGSGGGGSGGGGS

SEQ ID NO: 53 - protein, 245 aa, Artificial Sequence
  scFv
EVKLQQSGAELVRPGSSVKISCKASGYAFSSYWMNWVKQRPGQGLEWIGQIYPGDGDTNYNGKFKGQATLTADKSSSTAYMQLSGLTSEDSAVYFCARKTISSVVDFYFDYWGQGTTVTVSSGGGGSGGGGSGGGGSDIELTQSPKFMSTSVGDRVSVTCKASQNVGTNVAWYQQKPGQSPKPLIYSATYRNSGVPDRFTGSGSGTDFTLTITNVQSKDLADYFCQQYNRYPYTSGGGTKLEIKR

SEQ ID NO: 54 - protein, 12 aa, Artificial Sequence
  HC-CDR3
HYYYGGSYAMDY

SEQ ID NO: 55 - protein, 7 aa, Artificial Sequence
  LC-CDR2
HTSRLHS

SEQ ID NO: 56 - protein, 9 aa, Artificial Sequence
  LC-CDR3
QQGNTLPYT

SEQ ID NO: 57 - DNA, 735 bp, Artificial Sequence
  Sequence encoding scFv
gacatccagatgacccagaccacctccagcctgagcgccagcctgggcgaccgggtgaccatcagctgccgggccagccaggacatcagcaagtacctgaactggtatcagcagaagcccgacggcaccgtcaagctgctgatctaccacaccagccggctgcacagcggcgtgcccagccggtttagcggcagcggctccggcaccgactacagcctgaccatctccaacctggaacaggaagatatcgccacctacttttgccagcagggcaacacactgccctacacctttggcggcggaacaaagctggaaatcaccggcagcacctccggcagcggcaagcctggcagcggcgagggcagcaccaagggcgaggtgaagctgcaggaaagcggccctggcctggtggcccccagccagagcctgagcgtgacctgcaccgtgagcggcgtgagcctgcccgactacggcgtgagctggatccggcagccccccaggaagggcctggaatggctgggcgtgatctggggcagcgagaccacctactacaacagcgccctgaagagccggctgaccatcatcaaggacaacagcaagagccaggtgttcctgaagatgaacagcctgcagaccgacgacaccgccatctactactgcgccaagcactactactacggcggcagctacgccatggactactggggccagggcaccagcgtgaccgtgagcagc

SEQ ID NO: 58 - protein, 18 aa, Artificial Sequence
  Linker
GSTSGSGKPGSGEGSTKG

SEQ ID NO: 59 - protein, 567 aa, Artificial Sequence
  Human TGF-beta receptor type-2 (TGFR2) isoform 1
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQQKLSSTWETGKTRKLMEFSEHCAIILEDDRSDISSTCANNINHNTELLPIELDTLVGKGRFAEVYKAKLKQNTSEQFETVAVKIFPYEEYASWKTEKDIFSDINLKHENILQFLTAEERKTELGKQYWLITAFHAKGNLQEYLTRHVISWEDLRKLGSSLARGIAHLHSDHTPCGRPKMPIVHRDLKSSNILVKNDLTCCLCDFGLSLRLDPTLSVDDLANSGQVGTARYMAPEVLESRMNLENVESFKQTDVYSMALVLWEMTSRCNAVGEVKDYEPPFGSKVREHPCVESMKDNVLRDRGRPEIPSFWLNHQGIQMVCETLTECWDHDPEARLTAQCVAERFSELEHLDRLSGRSCSEEKIPEDGSLNTTK

SEQ ID NO: 60 - protein, 592 aa, Artificial Sequence
  Human TGF-beta receptor type-2 (TGFR2) isoform 2
MGRGLLRGLWPLHIVLWTRIASTIPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQQKLSSTWETGKTRKLMEFSEHCAIILEDDRSDISSTCANNINHNTELLPIELDTLVGKGRFAEVYKAKLKQNTSEQFETVAVKIFPYEEYASWKTEKDIFSDINLKHENILQFLTAEERKTELGKQYWLITAFHAKGNLQEYLTRHVISWEDLRKLGSSLARGIAHLHSDHTPCGRPKMPIVHRDLKSSNILVKNDLTCCLCDFGLSLRLDPTLSVDDLANSGQVGTARYMAPEVLESRMNLENVESFKQTDVYSMALVLWEMTSRCNAVGEVKDYEPPFGSKVREHPCVESMKDNVLRDRGRPEIPSFWLNHQGIQMVCETLTECWDHDPEARLTAQCVAERFSELEHLDRLSGRSCSEEKIPEDGSLNTTK

SEQ ID NO: 61 - DNA, 4629 bp, Artificial Sequence
  Human TGF-beta receptor type-2 (TGFR2) transcript variant B
ggagagggagaaggctctcgggcggagagaggtcctgcccagctgttggcgaggagtttcctgtttcccccgcagcgctgagttgaagttgagtgagtcactcgcgcgcacggagcgacgacacccccgcgcgtgcacccgctcgggacaggagccggactcctgtgcagcttccctcggccgccgggggcctccccgcgcctcgccggcctccaggccccctcctggctggcgagcgggcgccacatctggcccgcacatctgcgctgccggcccggcgcggggtccggagagggcgcggcgcggaggcgcagccaggggtccgggaaggcgccgtccgctgcgctgggggctcggtctatgacgagcagcggggtctgccatgggtcgggggctgctcaggggcctgtggccgctgcacatcgtcctgtggacgcgtatcgccagcacgatcccaccgcacgttcagaagtcggttaataacgacatgatagtcactgacaacaacggtgcagtcaagtttccacaactgtgtaaattttgtgatgtgagattttccacctgtgacaaccagaaatcctgcatgagcaactgcagcatcacctccatctgtgagaagccacaggaagtctgtgtggctgtatggagaaagaatgacgagaacataacactagagacagtttgccatgaccccaagctcccctaccatgactttattctggaagatgctgcttctccaaagtgcattatgaaggaaaaaaaaaagcctggtgagactttcttcatgtgttcctgtagctctgatgagtgcaatgacaacatcatcttctcagaagaatataacaccagcaatcctgacttgttgctagtcatatttcaagtgacaggcatcagcctcctgccaccactgggagttgccatatctgtcatcatcatcttctactgctaccgcgttaaccggcagcagaagctgagttcaacctgggaaaccggcaagacgcggaagctcatggagttcagcgagcactgtgccatcatcctggaagatgaccgctctgacatcagctccacgtgtgccaacaacatcaaccacaacacagagctgctgcccattgagctggacaccctggtggggaaaggtcgctttgctgaggtctataaggccaagctgaagcagaacacttcagagcagtttgagacagtggcagtcaagatctttccctatgaggagtatgcctcttggaagacagagaaggacatcttctcagacatcaatctgaagcatgagaacatactccagttcctgacggctgaggagcggaagacggagttggggaaacaatactggctgatcaccgccttccacgccaagggcaacctacaggagtacctgacgcggcatgtcatcagctgggaggacctgcgcaagctgggcagctccctcgcccgggggattgctcacctccacagtgatcacactccatgtgggaggcccaagatgcccatcgtgcacagggacctcaagagctccaatatcctcgtgaagaacgacctaacctgctgcctgtgtgactttgggctttccctgcgtctggaccctactctgtctgtggatgacctggctaacagtgggcaggtgggaactgcaagatacatggctccagaagtcctagaatccaggatgaatttggagaatgttgagtccttcaagcagaccgatgtctactccatggctctggtgctctgggaaatgacatctcgctgtaatgcagtgggagaagtaaaagattatgagcctccatttggttccaaggtgcgggagcacccctgtgtcgaaagcatgaaggacaacgtgttgagagatcgagggcgaccagaaattcccagcttctggctcaaccaccagggcatccagatggtgtgtgagacgttgactgagtgctgggaccacgacccagaggcccgtctcacagcccagtgtgtggcagaacgcttcagtgagctggagcatctggacaggctctcggggaggagctgctcggaggagaagattcctgaagacggctccctaaacactaccaaatagctcttctggggcaggctgggccatgtccaaagaggctgcccctctcaccaaagaacagaggcagcaggaagctgcccctgaactgatgcttcctggaaaaccaagggggtcactcccctccctgtaagctgtggggataagcagaaacaacagcagcagggagtgggtgacatagagcattctatgcctttgacattgtcataggataagctgtgttagcacttcctcaggaaatgagattgatttttacaatagccaataacatttgcactttattaatgcctgtatataaatatgaatagctatgttttatatatatatatatatatctatatatgtctatagctctatatatatagccataccttgaaaagagacaaggaaaaacatcaaatattcccaggaaattggttttattggagaactccagaaccaagcagagaaggaagggacccatgacagcattagcatttgacaatcacacatgcagtggttctctgactgtaaaacagtgaactttgcatgaggaaagaggctccatgtctcacagccagctatgaccacattgcacttgcttttgcaaaataatcattccctgcctagcacttctcttctggccatggaactaagtacagtggcactgtttgaggaccagtgttcccggggttcctgtgtgcccttatttctcctggacttttcatttaagctccaagccccaaatctggggggctagtttagaaactctccctcaacctagtttagaaactctaccccatctttaataccttgaatgttttgaaccccactttttaccttcatgggttgcagaaaaatcagaacagatgtccccatccatgcgattgccccaccatctactaatgaaaaattgttctttttttcatctttcccctgcacttatgttactattctctgctcccagccttcatccttttctaaaaaggagcaaattctcactctaggctttatcgtgtttactttttcattacacttgacttgattttctagttttctatacaaacaccaatgggttccatctttctgggctcctgattgctcaagcacagtttggcctgatgaagaggatttcaactacacaatactatcattgtcaggactatgacctcaggcactctaaacatatgttttgtttggtcagcacagcgtttcaaaaagtgaagccactttataaatatttggagattttgcaggaaaatctggatccccaggtaaggatagcagatggttttcagttatctccagtccacgttcacaaaatgtgaaggtgtggagacacttacaaagctgcctcacttctcactgtaaacattagctctttccactgcctacctggaccccagtctaggaattaaatctgcacctaaccaaggtcccttgtaagaaatgtccattcaagcagtcattctctgggtatataatatgattttgactaccttatctggtgttaagatttgaagttggccttttattggactaaaggggaactcctttaagggtctcagttagcccaagtttcttttgcttatatgttaatagttttaccctctgcattggagagaggagtgctttactccaagaagctttcctcatggttaccgttctctccatcatgccagccttctcaacctttgcagaaattactagagaggatttgaatgtgggacacaaaggtcccatttgcagttagaaaatttgtgtccacaaggacaagaacaaagtatgagctttaaaactccataggaaacttgttaatcaacaaagaagtgttaatgctgcaagtaatctcttttttaaaactttttgaagctacttattttcagccaaataggaatattagagagggactggtagtgagaatatcagctctgtttggatggtggaaggtctcattttattgagatttttaagatacatgcaaaggtttggaaatagaacctctaggcaccctcctcagtgtgggtgggctgagagttaaagacagtgtggctgcagtagcatagaggcgcctagaaattccacttgcaccgtagggcatgctgataccatcccaatagctgttgcccattgacctctagtggtgagtttctagaatactggtccattcatgagatattcaagattcaagagtattctcacttctgggttatcagcataaactggaatgtagtgtcagaggatactgtggcttgttttgtttatgtttttttttcttattcaagaaaaaagaccaaggaataacattctgtagttcctaaaaatactgacttttttcactactatacataaagggaaagttttattcttttatggaacacttcagctgtactcatgtattaaaataggaatgtgaatgctatatactctttttatatcaaaagtctcaagcacttatttttattctatgcattgtttgtcttttacataaataaaatgtttattagattgaataaagcaaaatactcaggtgagcatcctgcctcctgttcccattcctagtagctaaa

SEQ ID NO: 62 - DNA, 4704 bp, Artificial Sequence
  Human TGF-beta receptor type-2 (TGFR2) transcript variant A
ggagagggagaaggctctcgggcggagagaggtcctgcccagctgttggcgaggagtttcctgtttcccccgcagcgctgagttgaagttgagtgagtcactcgcgcgcacggagcgacgacacccccgcgcgtgcacccgctcgggacaggagccggactcctgtgcagcttccctcggccgccgggggcctccccgcgcctcgccggcctccaggccccctcctggctggcgagcgggcgccacatctggcccgcacatctgcgctgccggcccggcgcggggtccggagagggcgcggcgcggaggcgcagccaggggtccgggaaggcgccgtccgctgcgctgggggctcggtctatgacgagcagcggggtctgccatgggtcgggggctgctcaggggcctgtggccgctgcacatcgtcctgtggacgcgtatcgccagcacgatcccaccgcacgttcagaagtcggatgtggaaatggaggcccagaaagatgaaatcatctgccccagctgtaataggactgcccatccactgagacatattaataacgacatgatagtcactgacaacaacggtgcagtcaagtttccacaactgtgtaaattttgtgatgtgagattttccacctgtgacaaccagaaatcctgcatgagcaactgcagcatcacctccatctgtgagaagccacaggaagtctgtgtggctgtatggagaaagaatgacgagaacataacactagagacagtttgccatgaccccaagctcccctaccatgactttattctggaagatgctgcttctccaaagtgcattatgaaggaaaaaaaaaagcctggtgagactttcttcatgtgttcctgtagctctgatgagtgcaatgacaacatcatcttctcagaagaatataacaccagcaatcctgacttgttgctagtcatatttcaagtgacaggcatcagcctcctgccaccactgggagttgccatatctgtcatcatcatcttctactgctaccgcgttaaccggcagcagaagctgagttcaacctgggaaaccggcaagacgcggaagctcatggagttcagcgagcactgtgccatcatcctggaagatgaccgctctgacatcagctccacgtgtgccaacaacatcaaccacaacacagagctgctgcccattgagctggacaccctggtggggaaaggtcgctttgctgaggtctataaggccaagctgaagcagaacacttcagagcagtttgagacagtggcagtcaagatctttccctatgaggagtatgcctcttggaagacagagaaggacatcttctcagacatcaatctgaagcatgagaacatactccagttcctgacggctgaggagcggaagacggagttggggaaacaatactggctgatcaccgccttccacgccaagggcaacctacaggagtacctgacgcggcatgtcatcagctgggaggacctgcgcaagctgggcagctccctcgcccgggggattgctcacctccacagtgatcacactccatgtgggaggcccaagatgcccatcgtgcacagggacctcaagagctccaatatcctcgtgaagaacgacctaacctgctgcctgtgtgactttgggctttccctgcgtctggaccctactctgtctgtggatgacctggctaacagtgggcaggtgggaactgcaagatacatggctccagaagtcctagaatccaggatgaatttggagaatgttgagtccttcaagcagaccgatgtctactccatggctctggtgctctgggaaatgacatctcgctgtaatgcagtgggagaagtaaaagattatgagcctccatttggttccaaggtgcgggagcacccctgtgtcgaaagcatgaaggacaacgtgttgagagatcgagggcgaccagaaattcccagcttctggctcaaccaccagggcatccagatggtgtgtgagacgttgactgagtgctgggaccacgacccagaggcccgtctcacagcccagtgtgtggcagaacgcttcagtgagctggagcatctggacaggctctcggggaggagctgctcggaggagaagattcctgaagacggctccctaaacactaccaaatagctcttctggggcaggctgggccatgtccaaagaggctgcccctctcaccaaagaacagaggcagcaggaagctgcccctgaactgatgcttcctggaaaaccaagggggtcactcccctccctgtaagctgtggggataagcagaaacaacagcagcagggagtgggtgacatagagcattctatgcctttgacattgtcataggataagctgtgttagcacttcctcaggaaatgagattgatttttacaatagccaataacatttgcactttattaatgcctgtatataaatatgaatagctatgttttatatatatatatatatatctatatatgtctatagctctatatatatagccataccttgaaaagagacaaggaaaaacatcaaatattcccaggaaattggttttattggagaactccagaaccaagcagagaaggaagggacccatgacagcattagcatttgacaatcacacatgcagtggttctctgactgtaaaacagtgaactttgcatgaggaaagaggctccatgtctcacagccagctatgaccacattgcacttgcttttgcaaaataatcattccctgcctagcacttctcttctggccatggaactaagtacagtggcactgtttgaggaccagtgttcccggggttcctgtgtgcccttatttctcctggacttttcatttaagctccaagccccaaatctggggggctagtttagaaactctccctcaacctagtttagaaactctaccccatctttaataccttgaatgttttgaaccccactttttaccttcatgggttgcagaaaaatcagaacagatgtccccatccatgcgattgccccaccatctactaatgaaaaattgttctttttttcatctttcccctgcacttatgttactattctctgctcccagccttcatccttttctaaaaaggagcaaattctcactctaggctttatcgtgtttactttttcattacacttgacttgattttctagttttctatacaaacaccaatgggttccatctttctgggctcctgattgctcaagcacagtttggcctgatgaagaggatttcaactacacaatactatcattgtcaggactatgacctcaggcactctaaacatatgttttgtttggtcagcacagcgtttcaaaaagtgaagccactttataaatatttggagattttgcaggaaaatctggatccccaggtaaggatagcagatggttttcagttatctccagtccacgttcacaaaatgtgaaggtgtggagacacttacaaagctgcctcacttctcactgtaaacattagctctttccactgcctacctggaccccagtctaggaattaaatctgcacctaaccaaggtcccttgtaagaaatgtccattcaagcagtcattctctgggtatataatatgattttgactaccttatctggtgttaagatttgaagttggccttttattggactaaaggggaactcctttaagggtctcagttagcccaagtttcttttgcttatatgttaatagttttaccctctgcattggagagaggagtgctttactccaagaagctttcctcatggttaccgttctctccatcatgccagccttctcaacctttgcagaaattactagagaggatttgaatgtgggacacaaaggtcccatttgcagttagaaaatttgtgtccacaaggacaagaacaaagtatgagctttaaaactccataggaaacttgttaatcaacaaagaagtgttaatgctgcaagtaatctcttttttaaaactttttgaagctacttattttcagccaaataggaatattagagagggactggtagtgagaatatcagctctgtttggatggtggaaggtctcattttattgagatttttaagatacatgcaaaggtttggaaatagaacctctaggcaccctcctcagtgtgggtgggctgagagttaaagacagtgtggctgcagtagcatagaggcgcctagaaattccacttgcaccgtagggcatgctgataccatcccaatagctgttgcccattgacctctagtggtgagtttctagaatactggtccattcatgagatattcaagattcaagagtattctcacttctgggttatcagcataaactggaatgtagtgtcagaggatactgtggcttgttttgtttatgtttttttttcttattcaagaaaaaagaccaaggaataacattctgtagttcctaaaaatactgacttttttcactactatacataaagggaaagttttattcttttatggaacacttcagctgtactcatgtattaaaataggaatgtgaatgctatatactctttttatatcaaaagtctcaagcacttatttttattctatgcattgtttgtcttttacataaataaaatgtttattagattgaataaagcaaaatactcaggtgagcatcctgcctcctgttcccattcctagtagctaaa

SEQ ID NO: 63 - RNA, 20 nt, Artificial Sequence
  TGFBR2 targeting domain sequence 1
gcagaccgaugucuacucca

SEQ ID NO: 64 - RNA, 20 nt, Artificial Sequence
  TGFBR2 targeting domain sequence 2
ccccuaccaugacuuuauuc

SEQ ID NO: 65 - RNA, 20 nt, Artificial Sequence
  TGFBR2 targeting domain sequence 3
gacaucucgcuguaaugcag

SEQ ID NO: 66 - RNA, 20 nt, Artificial Sequence
  TGFBR2 targeting domain sequence 4
cacaugaagaaagucucacc

SEQ ID NO: 67 - RNA, 20 nt, Artificial Sequence
  TGFBR2 targeting domain sequence 5
augauagucacugacaacaa

SEQ ID NO: 68 - RNA, 20 nt, Artificial Sequence
  TGFBR2 targeting domain sequence 6
cuccaucugugagaagccac

SEQ ID NO: 69 - DNA, 610 bp, Artificial Sequence
  TGFBR2 5′ homology arm sequence 1
tacatgcagattttttgaaggcagaagctgtgtcattttttttcatgttcccaatgtcctgagcttagataacactcagtaaatggtttgtctttttatttggcaatattgaggacctgctgtgtgctaagtgcagtttacagtagtgaagaagacatggtaccttccagcatggagttccctgtccgtgggggatggcaagagtagggaaagacagatgtgaaatcaagaggtagagtcatagttcatttagtttaagttgtactgaattgttacctaggaaaagtataaggtgctatgaaaatgtataaaataagacagttttccaagtttttctaggcctctcttaagcagtgacatttaagctgaagtttgaaggaagagcaggggatgacgaacagatggccagaggcagggaaggctgaacgagcatgcacttgcatccctgaaataaaaattaacaatatcgtatctacaaaaactatgcagatgctaaaatctatagatgctcaggcatgaacccacttcctgacagtacttacctaccacatccaactccttctctccttgttttgtttccccatcagaatataacaccagcaatcctgac

SEQ ID NO: 70 - DNA, 600 bp, Artificial Sequence
  TGFBR2 5′ homology arm sequence 2
gtgcagtttacagtagtgaagaagacatggtaccttccagcatggagttccctgtccgtgggggatggcaagagtagggaaagacagatgtgaaatcaagaggtagagtcatagttcatttagtttaagttgtactgaattgttacctaggaaaagtataaggtgctatgaaaatgtataaaataagacagttttccaagtttttctaggcctctcttaagcagtgacatttaagctgaagtttgaaggaagagcaggggatgacgaacagatggccagaggcagggaaggctgaacgagcatgcacttgcatccctgaaataaaaattaacaatatcgtatctacaaaaactatgcagatgctaaaatctatagatgctcaggcatgaacccacttcctgacagtacttacctaccacatccaactccttctctccttgttttgtttccccatcagaatataacaccagcaatcctgacttgttgctagtcatatttcaagtgacaggcatcagcctcctgccaccactgggagttgccatatctgtcatcatcatcttctactgctaccgcgttaaccggcagcagaagctgagttca

SEQ ID NO: 71 - DNA, 600 bp, Artificial Sequence
  TGFBR2 5′ homology arm sequence 3
atggagttcagcgagcactgtgccatcatcctggaagatgaccgctctgacatcagctccacgtgtgccaacaacatcaaccacaacacagagctgctgcccattgagctggacaccctggtggggaaaggtcgctttgctgaggtctataaggccaagctgaagcagaacacttcagagcagtttgagacagtggcagtcaagatctttccctatgaggagtatgcctcttggaagacagagaaggacatcttctcagacatcaatctgaagcatgagaacatactccagttcctgacggctgaggagcggaagacggagttggggaaacaatactggctgatcaccgccttccacgccaagggcaacctacaggagtacctgacgcggcatgtcatcagctgggaggacctgcgcaagctgggcagctccctcgcccgggggattgctcacctccacagtgatcacactccatgtgggaggcccaagatgcccatcgtgcacagggacctcaagagctccaatatcctcgtgaagaacgacctaacctgctgcctgtgtgactttgggctttccctgcgtctggaccctactctgtct

SEQ ID NO: 72 - DNA, 600 bp, Artificial Sequence
  TGFBR2 3′ homology arm sequence 1
gtaagttagagctagtgctagatcccctttaccttgagcctggcctcaccctacctcttgatccatatctcctggctcttatctcaaacagccctgtactctggacactggtctagggaatctagccaaagtatggagtctgccttgagcatactctgctctgtcctgcctgagcatttttgctaatggacagcatttctcctcctatcttcaaatccttcccagttcagcacattttttcctcctggatcaatcctcatttctcttccagcaaatgttttttctttgtttcaagcactgttagtactttacctctattttttccctctcttatggttgtactcagtcctttctgctctatactagctgtagttgtgttggtttctttgtattaaaagcatcgtggaaggcaatctccctgaagtccaaatctacatccacatggtcacccaagatatgtagcacaatgccttgaacattgaaagtaaaataagtacttgtcgactgagtgagcacttccactcttgaagcactctcacagattaaaatggaaatgtttttggctaagaaactattggaaggtgattggaaatcaccaca

SEQ ID NO: 73 - RNA, 20 nt, Artificial Sequence
  TGFBR2 targeting domain sequence
guggaugaccuggcuaacag

SEQ ID NO: 74 - DNA, 20 bp, Artificial Sequence
  TGFBR2 target sequence 1
gcagaccgatgtctactcca

SEQ ID NO: 75 - DNA, 20 bp, Artificial Sequence
  TGFBR2 target sequence 2
gacatctcgctgtaatgcag

SEQ ID NO: 76 - DNA, 20 bp, Artificial Sequence
  TGFBR2 target sequence 3
acagtgatcacactccatgt

SEQ ID NO: 77 - DNA, 1189 bp, Homo sapiens
  EF1alpha promoter
cgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaa

SEQ ID NO: 78 - DNA, 25 bp, Artificial Sequence
  human HBB splice acceptor site
ctgacctcttctcttcctcccacag

SEQ ID NO: 79 - DNA, 13 bp, Artificial Sequence
  Human IgG splice acceptor site
tttctctccacag

SEQ ID NO: 80 - DNA, 20 bp, Artificial Sequence
  TGFBR2 target sequence A
gtagctctgatgagtgcaat

SEQ ID NO: 81 - DNA, 20 bp, Artificial Sequence
  TGFBR2 target sequence B
atgaatctcttcactctagg

SEQ ID NO: 82 - protein, 107 aa, Artificial Sequence
  FKBP
GVQVETISPGDGRTFPKRGQTCVVHYTGMLEDGKKMDSSRDRNKPFKFMLGKQEVIRGWEEGVAQMSVGQRAKLTISPDYAYGATGHPGIIPPHATLVFDVELLKLE

SEQ ID NO: 83 - protein, 107 aa, Artificial Sequence
  FKBP12v36
GVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLE

SEQ ID NO: 84 - protein, 16 aa, Artificial Sequence
  human C-Src acylation motif
MGSNKSKPKDASQRRR

SEQ ID NO: 85 - protein, 5 aa, Artificial Sequence
  dual acylation motif
  VARIANT (4)...(4): Xaa is any amino acid
MGCXC

SEQ ID NO: 86 - protein, 4 aa, Artificial Sequence
  CAAX motif
  VARIANT (4)...(4): Xaa is any amino acid
CAAX

SEQ ID NO: 87 - DNA, 20 bp, Artificial Sequence
  TGFBR2 target sequence C
acaggagtacctgacgcggc

SEQ ID NO: 88 - DNA, 20 bp, Artificial Sequence
  TGFBR2 target sequence D
ctgttagccaggtcatccac

SEQ ID NO: 89 - DNA, 20 bp, Artificial Sequence
  TGFBR2 target sequence E
gggtgtccagctcaatgggc

SEQ ID NO: 90 - DNA, 20 bp, Artificial Sequence
  TGFBR2 target sequence F
tcataatgcactttggagaa

SEQ ID NO: 91 - DNA, 20 bp, Artificial Sequence
  TGFBR2 target sequence G
tgactttattctggaagatg

SEQ ID NO: 92 - DNA, 20 bp, Artificial Sequence
  TGFBR2 target sequence 4
ggccgctgcacatcgtcctg

SEQ ID NO: 93 - DNA, 20 bp, Artificial Sequence
  TGFBR2 target sequence 5
gcggggtctgccatgggtcg

SEQ ID NO: 94 - DNA, 20 bp, Artificial Sequence
  TGFBR2 target sequence 6
agttgctcatgcaggatttc

SEQ ID NO: 95 - DNA, 20 bp, Artificial Sequence
  TGFBR2 target sequence 7
aagtcatggtaggggagctt

SEQ ID NO: 96 - DNA, 20 bp, Artificial Sequence
  TGFBR2 target sequence 8
agtcatggtaggggagcttg

SEQ ID NO: 97 - RNA, 96 nt, Artificial Sequence
  exemplary gRNA complementary domain
  modified_base (1)...(20): a, c, u, g, unknown or other
  modified_base (1)...(20): n is a, c, g, or u
nnnnnnnnnnnnnnnnnnnnguuuuagagcuagaaauagcaaguuaaaauaaggcuaguccguuaucaacuugaaaaaguggcaccgagucggugc

SEQ ID NO: 98 - RNA, 104 nt, Artificial Sequence
  exemplary gRNA complementary domain
  modified_base (1)...(20): a, c, u, g, unknown or other
  modified_base (1)...(20): n is a, c, g, or u
nnnnnnnnnnnnnnnnnnnnguuuuagagcuaugcugaaaagcauagcaaguuaaaauaaggcuaguccguuaucaacuugaaaaaguggcaccgagucggugc

SEQ ID NO: 99 - RNA, 106 nt, Artificial Sequence
  exemplary gRNA complementary domain
  modified_base (1)...(20): a, c, u, g, unknown or other
  modified_base (1)...(20): n is a, c, g, or u
nnnnnnnnnnnnnnnnnnnnguuuuagagcuaugcuggaaacagcauagcaaguuaaaauaaggcuaguccguuaucaacuugaaaaaguggcaccgagucggugc

SEQ ID NO: 100 - RNA, 116 nt, Artificial Sequence
  exemplary gRNA complementary domain
  modified_base (1)...(20): a, c, u, g, unknown or other
  modified_base (1)...(20): n is a, c, g, or u
nnnnnnnnnnnnnnnnnnnnguuuuagagcuaugcuguuuuggaaacaaaacagcauagcaaguuaaaauaaggcuaguccguuaucaacuugaaaaaguggcaccgagucggugc

SEQ ID NO: 101 - RNA, 96 nt, Artificial Sequence
  exemplary gRNA complementary domain
  modified_base (1)...(20): a, c, u, g, unknown or other
  modified_base (1)...(20): n is a, c, g, or u
nnnnnnnnnnnnnnnnnnnnguauuagagcuagaaauagcaaguuaauauaaggcuaguccguuaucaacuugaaaaaguggcaccgagucggugc

SEQ ID NO: 102 - RNA, 96 nt, Artificial Sequence
  exemplary gRNA complementary domain
  modified_base (1)...(20): a, c, u, g, unknown or other
  modified_base (1)...(20): n is a, c, g, or u
nnnnnnnnnnnnnnnnnnnnguuuaagagcuagaaauagcaaguuuaaauaaggcuaguccguuaucaacuugaaaaaguggcaccgagucggugc

SEQ ID NO: 103 - RNA, 116 nt, Artificial Sequence
  exemplary gRNA complementary domain
  modified_base (1)...(20): a, c, u, g, unknown or other
  modified_base (1)...(20): n is a, c, g, or u
nnnnnnnnnnnnnnnnnnnnguauuagagcuaugcuguauuggaaacaauacagcauagcaaguuaauauaaggcuaguccguuaucaacuugaaaaaguggcaccgagucggugc

SEQ ID NO: 104 - RNA, 47 nt, Artificial Sequence
  exemplary proximal and tail domain
aaggcuaguccguuaucaacuugaaaaaguggcaccgagucggugcu

SEQ ID NO: 105 - RNA, 49 nt, Artificial Sequence
  exemplary proximal and tail domain
aaggcuaguccguuaucaacuugaaaaaguggcaccgagucgguggugc

SEQ ID NO: 106 - RNA, 51 nt, Artificial Sequence
  exemplary proximal and tail domain
aaggcuaguccguuaucaacuugaaaaaguggcaccgagucggugcggauc

SEQ ID NO: 107 - RNA, 31 nt, Artificial Sequence
  exemplary proximal and tail domain
aaggcuaguccguuaucaacuugaaaaagug

SEQ ID NO: 108 - RNA, 18 nt, Artificial Sequence
  exemplary proximal and tail domain
aaggcuaguccguuauca

SEQ ID NO: 109 - RNA, 12 nt, Artificial Sequence
  exemplary proximal and tail domain
aaggcuaguccg

SEQ ID NO: 110 - RNA, 102 nt, Artificial Sequence
  exemplary chimeric gRNA
  modified_base (1)...(20): a, c, u, g, unknown or other
  modified_base (1)...(20): n is a, c, g, or u
nnnnnnnnnnnnnnnnnnnnguuuuagagcuagaaauagcaaguuaaaauaaggcuaguccguuaucaacuugaaaaaguggcaccgagucggugcuuuuuu

SEQ ID NO: 111 - RNA, 102 nt, Artificial Sequence
  exemplary chimeric gRNA
  modified_base (1)...(20): a, c, u, g, unknown or other
  modified_base (1)...(20): n is a, c, g, or u
nnnnnnnnnnnnnnnnnnnnguuuuaguacucuggaaacagaaucuacuaaaacaaggcaaaaugccguguuuaucucgucaacuuguuggcgagauuuuuu

SEQ ID NO: 112 - protein, 1344 aa, streptococcus mutans
  Cas9
KKPYSIGLDIGTNSVGWAVVTDDYKVPAKKMKVLGNTDKSHIEKNLLGALLFDSGNTAEDRRLKRTARRRYTRRRNRILYLQEIFSEEMGKVDDSFFHRLEDSFLVTEDKRGERHPIFGNLEEEVKYHENFPTIYHLRQYLADNPEKVDLRLVYLALAHIIKFRGHFLIEGKFDTRNNDVQRLFQEFLAVYDNTFENSSLQEQNVQVEEILTDKISKSAKKDRVLKLFPNEKSNGRFAEFLKLIVGNQADFKKHFELEEKAPLQFSKDTYEEELEVLLAQIGDNYAELFLSAKKLYDSILLSGILTVTDVGTKAPLSASMIQRYNEHQMDLAQLKQFIRQKLSDKYNEVFSDVSKDGYAGYIDGKTNQEAFYKYLKGLLNKIEGSGYFLDKIEREDFLRKQRTFDNGSIPHQIHLQEMRAIIRRQAEFYPFLADNQDRIEKLLTFRIPYYVGPLARGKSDFAWLSRKSADKITPWNFDEIVDKESSAEAFINRMTNYDLYLPNQKVLPKHSLLYEKFTVYNELTKVKYKTEQGKTAFFDANMKQEIFDGVFKVYRKVTKDKLMDFLEKEFDEFRIVDLTGLDKENKVFNASYGTYHDLCKILDKDFLDNSKNEKILEDIVLTLTLFEDREMIRKRLENYSDLLTKEQVKKLERRHYTGWGRLSAELIHGIRNKESRKTILDYLIDDGNSNRNFMQLINDDALSFKEEIAKAQVIGETDNLNQVVSDIAGSPAIKKGILQSLKIVDELVKIMGHQPENIVVEMARENQFTNQGRRNSQQRLKGLTDSIKEFGSQILKEHPVENSQLQNDRLFLYYLQNGRDMYTGEELDIDYLSQYDIDHIIPQAFIKDNSIDNRVLTSSKENRGKSDDVPSKDVVRKMKSYWSKLLSAKLITQRKFDNLTKAERGGLTDDDKAGFIKRQLVETRQITKHVARILDERFNTETDENNKKIRQVKIVTLKSNLVSNFRKEFELYKVREINDYHHAHDAYLNAVIGKALLGVYPQLEPEFVYGDYPHFHGHKENKATAKKFFYSNIMNFFKKDDVRTDKNGEIIWKKDEHISNIKKVLSYPQVNIVKKVEEQTGGFSKESILPKGNSDKLIPRKTKKFYWDTKKYGGFDSPIVAYSILVIADIEKGKSKKLKTVKALVGVTIMEKMTFERDPVAFLERKGYRNVQEENIIKLPKYSLFKLENGRKRLLASARELQKGNEIVLPNHLGTLLYHAKNIHKVDEPKHLDYVDKHKDEFKELLDVVSNFSKKYTLAEGNLEKIKELYAQNNGEDLKELASSFINLLTFTAIGAPATFKFFDKNIDRKRYTSTTEILNATLIHQSITGLYETRIDLNKLGGD

SEQ ID NO: 113 - protein, 1367 aa, Streptococcus pyogenes
  Cas9
DKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 114 - protein, 1387 aa, Streptococcus thermophilus
  Cas9
TKPYSIGLDIGTNSVGWAVTTDNYKVPSKKMKVLGNTSKKYIKKNLLGVLLFDSGITAEGRRLKRTARRRYTRRRNRILYLQEIFSTEMATLDDAFFQRLDDSFLVPDDKRDSKYPIFGNLVEEKAYHDEFPTIYHLRKYLADSTKKADLRLVYLALAHMIKYRGHFLIEGEFNSKNNDIQKNFQDFLDTYNAIFESDLSLENSKQLEEIVKDKISKLEKKDRILKLFPGEKNSGIFSEFLKLIVGNQADFRKCFNLDEKASLHFSKESYDEDLETLLGYIGDDYSDVFLKAKKLYDAILLSGFLTVTDNETEAPLSSAMIKRYNEHKEDLALLKEYIRNISLKTYNEVFKDDTKNGYAGYIDGKTNQEDFYVYLKKLLAEFEGADYFLEKIDREDFLRKQRTFDNGSIPYQIHLQEMRAILDKQAKFYPFLAKNKERIEKILTFRIPYYVGPLARGNSDFAWSIRKRNEKITPWNFEDVIDKESSAEAFINRMTSFDLYLPEEKVLPKHSLLYETFNVYNELTKVRFIAESMRDYQFLDSKQKKDIVRLYFKDKRKVTDKDIIEYLHAIYGYDGIELKGIEKQFNSSLSTYHDLLNIINDKEFLDDSSNEAIIEEIIHTLTIFEDREMIKQRLSKFENIFDKSVLKKLSRRHYTGWGKLSAKLINGIRDEKSGNTILDYLIDDGISNRNFMQLIHDDALSFKKKIQKAQIIGDEDKGNIKEVVKSLPGSPAIKKGILQSIKIVDELVKVMGGRKPESIVVEMARENQYTNQGKSNSQQRLKRLEKSLKELGSKILKENIPAKLSKIDNNALQNDRLYLYYLQNGKDMYTGDDLDIDRLSNYDIDHIIPQAFLKDNSIDNKVLVSSASNRGKSDDVPSLEVVKKRKTFWYQLLKSKLISQRKFDNLTKAERGGLSPEDKAGFIQRQLVETRQITKHVARLLDEKFNNKKDENNRAVRTVKIITLKSTLVSQFRKDFELYKVREINDFHHAHDAYLNAVVASALLKKYPKLEPEFVYGDYPKYNSFRERKSATEKVYFYSNIMNIFKKSISLADGRVIERPLIEVNEETGESVWNKESDLATVRRVLSYPQVNVVKKVEEQNHGLDRGKPKGLFNANLSSKPKPNSNENLVGAKEYLDPKKYGGYAGISNSFTVLVKGTIEKGAKKKITNVLEFQGISILDRINYRKDKLNFLLEKGYKDIELIIELPKYSLFELSDGSRRMLASILSTNNKRGEIHKGNQIFLSQKFVKLLYHAKRISNTINENHRKYVENHKKEFEELFYYILEFNENYVGAKKNGKLLNSAFQSWQNHSIDELCSSFIGPTGSERKGLFELTSRGSAADFEFLGVKIPRYRDYTPSSLLKDATLIHQSVTGLYETRIDLAKLGEG

SEQ ID NO: 115 - protein, 1333 aa, Listeria innocua
  Cas9
KKPYTIGLDIGTNSVGWAVLTDQYDLVKRKMKIAGDSEKKQIKKNFWGVRLFDEGQTAADRRMARTARRRIERRRNRISYLQGIFAEEMSKTDANFFCRLSDSFYVDNEKRNSRHPFFATIEEEVEYHKNYPTIYHLREELVNSSEKADLRLVYLALAHIIKYRGNFLIEGALDTQNTSVDGIYKQFIQTYNQVFASGIEDGSLKKLEDNKDVAKILVEKVTRKEKLERILKLYPGEKSAGMFAQFISLIVGSKGNFQKPFDLIEKSDIECAKDSYEEDLESLLALIGDEYAELFVAAKNAYSAVVLSSIITVAETETNAKLSASMIERFDTHEEDLGELKAFIKLHLPKHYEEIFSNTEKHGYAGYIDGKTKQADFYKYMKMTLENIEGADYFIAKIEKENFLRKQRTFDNGAIPHQLHLEELEAILHQQAKYYPFLKENYDKIKSLVTFRIPYFVGPLANGQSEFAWLTRKADGEIRPWNIEEKVDFGKSAVDFIEKMTNKDTYLPKENVLPKHSLCYQKYLVYNELTKVRYINDQGKTSYFSGQEKEQIFNDLFKQKRKVKKKDLELFLRNMSHVESPTIEGLEDSFNSSYSTYHDLLKVGIKQEILDNPVNTEMLENIVKILTVFEDKRMIKEQLQQFSDVLDGVVLKKLERRHYTGWGRLSAKLLMGIRDKQSHLTILDYLMNDDGLNRNLMQLINDSNLSFKSIIEKEQVTTADKDIQSIVADLAGSPAIKKGILQSLKIVDELVSVMGYPPQTIVVEMARENQTTGKGKNNSRPRYKSLEKAIKEFGSQILKEHPTDNQELRNNRLYLYYLQNGKDMYTGQDLDIHNLSNYDIDHIVPQSFITDNSIDNLVLTSSAGNREKGDDVPPLEIVRKRKVFWEKLYQGNLMSKRKFDYLTKAERGGLTEADKARFIHRQLVETRQITKNVANILHQRFNYEKDDHGNTMKQVRIVTLKSALVSQFRKQFQLYKVRDVNDYHHAHDAYLNGVVANTLLKVYPQLEPEFVYGDYHQFDWFKANKATAKKQFYTNIMLFFAQKDRIIDENGEILWDKKYLDTVKKVMSYRQMNIVKKTEIQKGEFSKATIKPKGNSSKLIPRKTNWDPMKYGGLDSPNMAYAVVIEYAKGKNKLVFEKKIIRVTIMERKAFEKDEKAFLEEQGYRQPKVLAKLPKYTLYECEEGRRRMLASANEAQKGNQQVLPNHLVTLLHHAANCEVSDGKSLDYIESNREMFAELLAHVSEFAKRYTLAEANLNKINQLFEQNKEGDIKAIAQSFVDLMAFNAMGAPASFKFFETTIERKRYNNLKELLNSTIIYQSITGLYESRKRLDD

SEQ ID NO: 116 - protein, 1082 aa, Neisseria meningitidis
  Cas9
MAAFKPNSINYILGLDIGIASVGWAMVEIDEEENPIRLIDLGVRVFERAEVPKTGDSLAMARRLARSVRRLTRRRAHRLLRTRRLLKREGVLQAANFDENGLIKSLPNTPWQLRAAALDRKLTPLEWSAVLLHLIKHRGYLSQRKNEGETADKELGALLKGVAGNAHALQTGDFRTPAELALNKFEKESGHIRNQRSDYSHTFSRKDLQAELILLFEKQKEFGNPHVSGGLKEGIETLLMTQRPALSGDAVQKMLGHCTFEPAEPKAAKNTYTAERFIWLTKLNNLRILEQGSERPLTDTERATLMDEPYRKSKLTYAQARKLLGLEDTAFFKGLRYGKDNAEASTLMEMKAYHAISRALEKEGLKDKKSPLNLSPELQDEIGTAFSLFKTDEDITGRLKDRIQPEILEALLKHISFDKFVQISLKALRRIVPLMEQGKRYDEACAEIYGDHYGKKNTEEKIYLPPIPADEIRNPVVLRALSQARKVINGVVRRYGSPARIHIETAREVGKSFKDRKEIEKRQEENRKDREKAAAKFREYFPNFVGEPKSKDILKLRLYEQQHGKCLYSGKEINLGRLNEKGYVEIDHALPFSRTWDDSFNNKVLVLGSENQNKGNQTPYEYFNGKDNSREWQEFKARVETSRFPRSKKQRILLQKFDEDGFKERNLNDTRYVNRFLCQFVADRMRLTGKGKKRVFASNGQITNLLRGFWGLRKVRAENDRHHALDAVVVACSTVAMQQKITRFVRYKEMNAFDGKTIDKETGEVLHQKTHFPQPWEFFAQEVMIRVFGKPDGKPEFEEADTLEKLRTLLAEKLSSRPEAVHEYVTPLFVSRAPNRKMSGQGHMETVKSAKRLDEGVSVLRVPLTQLKLKDLEKMVNREREPKLYEALKARLEAHKDDPAKAFAEPFYKYDKAGNRTQQVKAVRVEQVQKTGVWVRNHNGIADNATMVRVDVFEKGDKYYLVPIYSWQVAKGILPDRAVVQGKDEEDWQLIDDSFNFKFSLHPNDLVEVITKKARMFGYFASCHRGTGNINIRIHDLDHKIGKNGILEGIGVKTALSFQKYQIDELGKEIRPCRLKKRPPVR

SEQ ID NO: 117 - protein, 1368 aa, Streptococcus pyogenes [sequence omitted]

SEQ ID NO: 118 - DNA, 1205 bp, Artificial Sequence
  EF1alpha promoter
cgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcactagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgtggccttgcgcttaaggagccccttcgcctcgtgcttgagttgtggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatcagcacactggtatttcggtttttggggccgcgggcggcgacggggcccgtgcgtcccagcgcacatgttcggcgaggcggggcctgcgagcgcggccaccgagaatcggacgggggtagtctcaagctgcccggcctgctctggtgcctggcctcgcgccgccgtgtatcgccccgccctgggcggcaaggctggcccggtcggcaccagttgcgtgagcggaaagatggccgcttcccggccctgctgcagggagcacaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctccagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaaaactacccctaaaagccaaa

SEQ ID NO: 119 - DNA, 544 bp, Artificial Sequence
  Ef1alpha promoter with HTLV1 enhancer
ggatctgcgatcgctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctac

SEQ ID NO: 120 - DNA, 66 bp, Artificial Sequence
  P2A nucleotide sequence
ggatctggagcgacgaattttagtctactgaaacaagcgggagacgtggaggaaaaccctggacct

SEQ ID NO: 121 - DNA, 4107 bp, Streptococcus pyogenes
  Cas9 codon optimized nucleic acid sequence
atggataaaaagtacagcatcgggctggacatcggtacaaactcagtggggtgggccgtgattacggacgagtacaaggtaccctccaaaaaatttaaagtgctgggtaacacggacagacactctataaagaaaaatcttattggagccttgctgttcgactcaggcgagacagccgaagccacaaggttgaagcggaccgccaggaggcggtataccaggagaaagaaccgcatatgctacctgcaagaaatcttcagtaacgagatggcaaaggttgacgatagctttttccatcgcctggaagaatcctttcttgttgaggaagacaagaagcacgaacggcaccccatctttggcaatattgtcgacgaagtggcatatcacgaaaagtacccgactatctaccacctcaggaagaagctggtggactctaccgataaggcggacctcagacttatttatttggcactcgcccacatgattaaatttagaggacatttcttgatcgagggcgacctgaacccggacaacagtgacgtcgataagctgttcatccaacttgtgcagacctacaatcaactgttcgaagaaaaccctataaatgcttcaggagtcgacgctaaagcaatcctgtccgcgcgcctctcaaaatctagaagacttgagaatctgattgctcagttgcccggggaaaagaaaaatggattgtttggcaacctgatcgccctcagtctcggactgaccccaaatttcaaaagtaacttcgacctggccgaagacgctaagctccagctgtccaaggacacatacgatgacgacctcgacaatctgctggcccagattggggatcagtacgccgatctctttttggcagcaaagaacctgtccgacgccatcctgttgagcgatatcttgagagtgaacaccgaaattactaaagcaccccttagcgcatctatgatcaagcggtacgacgagcatcatcaggatctgaccctgctgaaggctcttgtgaggcaacagctccccgaaaaatacaaggaaatcttctttgaccagagcaaaaacggctacgctggctatatagatggtggggccagtcaggaggaattctataaattcatcaagcccattctcgagaaaatggacggcacagaggagttgctggtcaaacttaacagggaggacctgctgcggaagcagcggacctttgacaacgggtctatcccccaccagattcatctgggcgaactgcacgcaatcctgaggaggcaggaggatttttatccttttcttaaagataaccgcgagaaaatagaaaagattcttacattcaggatcccgtactacgtgggacctctcgcccggggcaattcacggtttgcctggatgacaaggaagtcagaggagactattacaccttggaacttcgaagaagtggtggacaagggtgcatctgcccagtctttcatcgagcggatgacaaattttgacaagaacctccctaatgagaaggtgctgcccaaacattctctgctctacgagtactttaccgtctacaatgaactgactaaagtcaagtacgtcaccgagggaatgaggaagccggcattccttagtggagaacagaagaaggcgattgtagacctgttgttcaagaccaacaggaaggtgactgtgaagcaacttaaagaagactactttaagaagatcgaatgttttgacagtgtggaaatttcaggggttgaagaccgcttcaatgcgtcattggggacttaccatgatcttctcaagatcataaaggacaaagacttcctggacaacgaagaaaatgaggatattctcgaagacatcgtcctcaccctgaccctgttcgaagacagggaaatgatagaagagcgcttgaaaacctatgcccacctcttcgacgataaagttatgaagcagctgaagcgcaggagatacacaggatggggaagattgtcaaggaagctgatcaatggaattagggataaacagagtggcaagaccatactggatttcctcaaatctgatggcttcgccaataggaacttcatgcaactgattcacgatgactctcttaccttcaaggaggacattcaaaaggctcaggtgagcgggcagggagactcccttcatgaacacatcgcgaatttggcaggttcccccgctattaaaaagggcatccttcaaactgtcaaggtggtggatgaattggtcaaggtaatgggcagacataagccagaaaatattgtgatcgagatggcccgcgaaaaccagaccacacagaagggccagaaaaatagtagagagcggatgaagaggatcgaggagggcatcaaagagctgggatctcagattctcaaagaacaccccgtagaaaacacacagctgcagaacgaaaaattgtacttgtactatctgcagaacggcagagacatgtacgtcgaccaagaacttgatattaatagactgtccgactatgacgtagaccatatcgtgccccagtccttcctgaaggacgactccattgataacaaagtcttgacaagaagcgacaagaacaggggtaaaagtgataatgtgcctagcgaggaggtggtgaaaaaaatgaagaactactggcgacagctgcttaatgcaaagctcattacacaacggaagttcgataatctgacgaaagcagagagaggtggcttgtctgagttggacaaggcagggtttattaagcggcagctggtggaaactaggcagatcacaaagcacgtggcgcagattttggacagccggatgaacacaaaatacgacgaaaatgataaactgatacgagaggtcaaagttatcacgctgaaaagcaagctggtgtccgattttcggaaagacttccagttctacaaagttcgcgagattaataactaccatcatgctcacgatgcgtacctgaacgctgttgtcgggaccgccttgataaagaagtacccaaagctggaatccgagttcgtatacggggattacaaagtgtacgatgtgaggaaaatgatagccaagtccgagcaggagattggaaaggccacagctaagtacttcttttattctaacatcatgaatttttttaagacggaaattaccctggccaacggagagatcagaaagcggccccttatagagacaaatggtgaaacaggtgaaatcgtctgggataagggcagggatttcgctactgtgaggaaggtgctgagtatgccacaggtaaatatcgtgaaaaaaaccgaagtacagaccggaggattttccaaggaaagcattttgcctaaaagaaactcagacaagctcatcgcccgcaagaaagattgggaccctaagaaatacgggggatttgactcacccaccgtagcctattctgtgctggtggtagctaaggtggaaaaaggaaagtctaagaagctgaagtccgtgaaggaactcttgggaatcactatcatggaaagatcatcctttgaaaagaaccctatcgatttcctggaggctaagggttacaaggaggtcaagaaagacctcatcattaaactgccaaaatactctctcttcgagctggaaaatggcaggaagagaatgttggccagcgccggagagctgcaaaagggaaacgagcttgctctgccctccaaatatgttaattttctctatctcgcttcccactatgaaaagctgaaagggtctcccgaagataacgagcagaagcagctgttcgtcgaacagcacaagcactatctggatgaaataatcgaacaaataagcgagttcagcaaaagggttatcctggcggatgctaatttggacaaagtactgtctgcttataacaagcaccgggataagcctattagggaacaagccgagaatataattcacctctttacactcacgaatctcggagcccccgccgccttcaaatactttgatacgactatcgaccggaaacggtataccagtaccaaagaggtcctcgatgccaccctcatccaccagtcaattactggcctgtacgaaacacggatcgacctctctcaactgggcggcgactag

SEQ ID NO: 122 - protein, 1368 aa, Streptococcus pyogenes [sequence omitted]

SEQ ID NO: 123 - DNA, 3249 bp, Neisseria meningitidis
  Cas9 codon optimized nucleic acid sequence
atggccgccttcaagcccaaccccatcaactacatcctgggcctggacatcggcatcgccagcgtgggctgggccatggtggagatcgacgaggacgagaaccccatctgcctgatcgacctgggtgtgcgcgtgttcgagcgcgctgaggtgcccaagactggtgacagtctggctatggctcgccggcttgctcgctctgttcggcgccttactcgccggcgcgctcaccgccttctgcgcgctcgccgcctgctgaagcgcgagggtgtgctgcaggctgccgacttcgacgagaacggcctgatcaagagcctgcccaacactccttggcagctgcgcgctgccgctctggaccgcaagctgactcctctggagtggagcgccgtgctgctgcacctgatcaagcaccgcggctacctgagccagcgcaagaacgagggcgagaccgccgacaaggagctgggtgctctgctgaagggcgtggccgacaacgcccacgccctgcagactggtgacttccgcactcctgctgagctggccctgaacaagttcgagaaggagagcggccacatccgcaaccagcgcggcgactacagccacaccttcagccgcaaggacctgcaggccgagctgatcctgctgttcgagaagcagaaggagttcggcaacccccacgtgagcggcggcctgaaggagggcatcgagaccctgctgatgacccagcgccccgccctgagcggcgacgccgtgcagaagatgctgggccactgcaccttcgagccagccgagcccaaggccgccaagaacacctacaccgccgagcgcttcatctggctgaccaagctgaacaacctgcgcatcctggagcagggcagcgagcgccccctgaccgacaccgagcgcgccaccctgatggacgagccctaccgcaagagcaagctgacctacgcccaggcccgcaagctgctgggtctggaggacaccgccttcttcaagggcctgcgctacggcaaggacaacgccgaggccagcaccctgatggagatgaaggcctaccacgccatcagccgcgccctggagaaggagggcctgaaggacaagaagagtcctctgaacctgagccccgagctgcaggacgagatcggcaccgccttcagcctgttcaagaccgacgaggacatcaccggccgcctgaaggaccgcatccagcccgagatcctggaggccctgctgaagcacatcagcttcgacaagttcgtgcagatcagcctgaaggccctgcgccgcatcgtgcccctgatggagcagggcaagcgctacgacgaggcctgcgccgagatctacggcgaccactacggcaagaagaacaccgaggagaagatctacctgcctcctatccccgccgacgagatccgcaaccccgtggtgctgcgcgccctgagccaggcccgcaaggtgatcaacggcgtggtgcgccgctacggcagccccgcccgcatccacatcgagaccgcccgcgaggtgggcaagagcttcaaggaccgcaaggagatcgagaagcgccaggaggagaaccgcaaggaccgcgagaaggccgccgccaagttccgcgagtacttccccaacttcgtgggcgagcccaagagcaaggacatcctgaagctgcgcctgtacgagcagcagcacggcaagtgcctgtacagcggcaaggagatcaacctgggccgcctgaacgagaagggctacgtggagatcgaccacgccctgcccttcagccgcacctgggacgacagcttcaacaacaaggtgctggtgctgggcagcgagaaccagaacaagggcaaccagaccccctacgagtacttcaacggcaaggacaacagccgcgagtggcaggagttcaaggcccgcgtggagaccagccgcttcccccgcagcaagaagcagcgcatcctgctgcagaagttcgacgaggacggcttcaaggagcgcaacctgaacgacacccgctacgtgaaccgcttcctgtgccagttcgtggccgaccgcatgcgcctgaccggcaagggcaagaagcgcgtgttcgccagcaacggccagatcaccaacctgctgcgcggcttctggggcctgcgcaaggtgcgcgccgagaacgaccgccaccacgccctggacgccgtggtggtggcctgcagcaccgtggccatgcagcagaagatcacccgcttcgtgcgctacaaggagatgaacgccttcgacggtaaaaccatcgacaaggagaccggcgaggtgctgcaccagaagacccacttcccccagccctgggagttcttcgcccaggaggtgatgatccgcgtgttcggcaagcccgacggcaagcccgagttcgaggaggccgacacccccgagaagctgcgcaccctgctggccgagaagctgagcagccgccctgaggccgtgcacgagtacgtgactcctctgttcgtgagccgcgcccccaaccgcaagatgagcggtcagggtcacatggagaccgtgaagagcgccaagcgcctggacgagggcgtgagcgtgctgcgcgtgcccctgacccagctgaagctgaaggacctggagaagatggtgaaccgcgagcgcgagcccaagctgtacgaggccctgaaggcccgcctggaggcccacaaggacgaccccgccaaggccttcgccgagcccttctacaagtacgacaaggccggcaaccgcacccagcaggtgaaggccgtgcgcgtggagcaggtgcagaagaccggcgtgtgggtgcgcaaccacaacggcatcgccgacaacgccaccatggtgcgcgtggacgtgttcgagaagggcgacaagtactacctggtgcccatctacagctggcaggtggccaagggcatcctgcccgaccgcgccgtggtgcagggcaaggacgaggaggactggcagctgatcgacgacagcttcaacttcaagttcagcctgcaccccaacgacctggtggaggtgatcaccaagaaggcccgcatgttcggctacttcgccagctgccaccgcggcaccggcaacatcaacatccgcatccacgacctggaccacaagatcggcaagaacggcatcctggagggcatcggcgtgaagaccgccctgagcttccagaagtaccagatcgacgagctgggcaaggagatccgcccctgccgcctgaagaagcgccctcctgtgcgctaa

SEQ ID NO: 124 - protein, 1082 aa, Neisseria meningitidis
  Cas9
MAAFKPNPINYILGLDIGIASVGWAMVEIDEDENPICLIDLGVRVFERAEVPKTGDSLAMARRLARSVRRLTRRRAHRLLRARRLLKREGVLQAADFDENGLIKSLPNTPWQLRAAALDRKLTPLEWSAVLLHLIKHRGYLSQRKNEGETADKELGALLKGVADNAHALQTGDFRTPAELALNKFEKESGHIRNQRGDYSHTFSRKDLQAELILLFEKQKEFGNPHVSGGLKEGIETLLMTQRPALSGDAVQKMLGHCTFEPAEPKAAKNTYTAERFIWLTKLNNLRILEQGSERPLTDTERATLMDEPYRKSKLTYAQARKLLGLEDTAFFKGLRYGKDNAEASTLMEMKAYHAISRALEKEGLKDKKSPLNLSPELQDEIGTAFSLFKTDEDITGRLKDRIQPEILEALLKHISFDKFVQISLKALRRIVPLMEQGKRYDEACAEIYGDHYGKKNTEEKIYLPPIPADEIRNPVVLRALSQARKVINGVVRRYGSPARIHIETAREVGKSFKDRKEIEKRQEENRKDREKAAAKFREYFPNFVGEPKSKDILKLRLYEQQHGKCLYSGKEINLGRLNEKGYVEIDHALPFSRTWDDSFNNKVLVLGSENQNKGNQTPYEYFNGKDNSREWQEFKARVETSRFPRSKKQRILLQKFDEDGFKERNLNDTRYVNRFLCQFVADRMRLTGKGKKRVFASNGQITNLLRGFWGLRKVRAENDRHHALDAVVVACSTVAMQQKITRFVRYKEMNAFDGKTIDKETGEVLHQKTHFPQPWEFFAQEVMIRVFGKPDGKPEFEEADTPEKLRTLLAEKLSSRPEAVHEYVTPLFVSRAPNRKMSGQGHMETVKSAKRLDEGVSVLRVPLTQLKLKDLEKMVNREREPKLYEALKARLEAHKDDPAKAFAEPFYKYDKAGNRTQQVKAVRVEQVQKTGVWVRNHNGIADNATMVRVDVFEKGDKYYLVPIYSWQVAKGILPDRAVVQGKDEEDWQLIDDSFNFKFSLHPNDLVEVITKKARMFGYFASCHRGTGNINIRIHDLDHKIGKNGILEGIGVKTALSFQKYQIDELGKEIRPCRLKKRPPVR

SEQ ID NO: 125 - DNA, 3159 bp, staphylococcus aureus
  Cas9 codon optimized nucleic acid sequence
atgaaaaggaactacattctggggctggacatcgggattacaagcgtggggtatgggattattgactatgaaacaagggacgtgatcgacgcaggcgtcagactgttcaaggaggccaacgtggaaaacaatgagggacggagaagcaagaggggagccaggcgcctgaaacgacggagaaggcacagaatccagagggtgaagaaactgctgttcgattacaacctgctgaccgaccattctgagctgagtggaattaatccttatgaagccagggtgaaaggcctgagtcagaagctgtcagaggaagagttttccgcagctctgctgcacctggctaagcgccgaggagtgcataacgtcaatgaggtggaagaggacaccggcaacgagctgtctacaaaggaacagatctcacgcaatagcaaagctctggaagagaagtatgtcgcagagctgcagctggaacggctgaagaaagatggcgaggtgagagggtcaattaataggttcaagacaagcgactacgtcaaagaagccaagcagctgctgaaagtgcagaaggcttaccaccagctggatcagagcttcatcgatacttatatcgacctgctggagactcggagaacctactatgagggaccaggagaagggagccccttcggatggaaagacatcaaggaatggtacgagatgctgatgggacattgcacctattttccagaagagctgagaagcgtcaagtacgcttataacgcagatctgtacaacgccctgaatgacctgaacaacctggtcatcaccagggatgaaaacgagaaactggaatactatgagaagttccagatcatcgaaaacgtgtttaagcagaagaaaaagcctacactgaaacagattgctaaggagatcctggtcaacgaagaggacatcaagggctaccgggtgacaagcactggaaaaccagagttcaccaatctgaaagtgtatcacgatattaaggacatcacagcacggaaagaaatcattgagaacgccgaactgctggatcagattgctaagatcctgactatctaccagagctccgaggacatccaggaagagctgactaacctgaacagcgagctgacccaggaagagatcgaacagattagtaatctgaaggggtacaccggaacacacaacctgtccctgaaagctatcaatctgattctggatgagctgtggcatacaaacgacaatcagattgcaatctttaaccggctgaagctggtcccaaaaaaggtggacctgagtcagcagaaagagatcccaaccacactggtggacgatttcattctgtcacccgtggtcaagcggagcttcatccagagcatcaaagtgatcaacgccatcatcaagaagtacggcctgcccaatgatatcattatcgagctggctagggagaagaacagcaaggacgcacagaagatgatcaatgagatgcagaaacgaaaccggcagaccaatgaacgcattgaagagattatccgaactaccgggaaagagaacgcaaagtacctgattgaaaaaatcaagctgcacgatatgcaggagggaaagtgtctgtattctctggaggccatccccctggaggacctgctgaacaatccattcaactacgaggtcgatcatattatccccagaagcgtgtccttcgacaattcctttaacaacaaggtgctggtcaagcaggaagagaactctaaaaagggcaataggactcctttccagtacctgtctagttcagattccaagatctcttacgaaacctttaaaaagcacattctgaatctggccaaaggaaagggccgcatcagcaagaccaaaaaggagtacctgctggaagagcgggacatcaacagattctccgtccagaaggattttattaaccggaatctggtggacacaagatacgctactcgcggcctgatgaatctgctgcgatcctatttccgggtgaacaatctggatgtgaaagtcaagtccatcaacggcgggttcacatcttttctgaggcgcaaatggaagtttaaaaaggagcgcaacaaagggtacaagcaccatgccgaagatgctctgattatcgcaaatgccgacttcatctttaaggagtggaaaaagctggacaaagccaagaaagtgatggagaaccagatgttcgaagagaagcaggccgaatctatgcccgaaatcgagacagaacaggagtacaaggagattttcatcactcctcaccagatcaagcatatcaaggatttcaaggactacaagtactctcaccgggtggataaaaagcccaacagagagctgatcaatgacaccctgtatagtacaagaaaagacgataaggggaataccctgattgtgaacaatctgaacggactgtacgacaaagataatgacaagctgaaaaagctgatcaacaaaagtcccgagaagctgctgatgtaccaccatgatcctcagacatatcagaaactgaagctgattatggagcagtacggcgacgagaagaacccactgtataagtactatgaagagactgggaactacctgaccaagtatagcaaaaaggataatggccccgtgatcaagaagatcaagtactatgggaacaagctgaatgcccatctggacatcacagacgattaccctaacagtcgcaacaaggtggtcaagctgtcactgaagccatacagattcgatgtctatctggacaacggcgtgtataaatttgtgactgtcaagaatctggatgtcatcaaaaaggagaactactatgaagtgaatagcaagtgctacgaagaggctaaaaagctgaaaaagattagcaaccaggcagagttcatcgcctccttttacaacaacgacctgattaagatcaatggcgaactgtatagggtcatcggggtgaacaatgatctgctgaaccgcattgaagtgaatatgattgacatcacttaccgagagtatctggaaaacatgaatgataagcgcccccctcgaattatcaaaacaattgcctctaagactcagagtatcaaaaagtactcaaccgacattctgggaaacctgtatgaggtgaagagcaaaaagcaccctcagattatcaaaaagggc

SEQ ID NO: 126 - protein, 1053 aa, Staphylococcus aureus
  Cas9
MKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 127 - DNA, 20 bp, Artificial Sequence
  TGFBR2 target sequence 9
attgcactcatcagagctac

SEQ ID NO: 128 - DNA, 20 bp, Artificial Sequence
  TGFBR2 target sequence 10
cctagagtgaagagattcat

SEQ ID NO: 129 - DNA, 20 bp, Artificial Sequence
  TGFBR2 target sequence 11
ccaatgaatctcttcactct

SEQ ID NO: 130 - DNA, 20 bp, Artificial Sequence
  TGFBR2 target sequence 12
aaagtcatggtaggggagct

SEQ ID NO: 131 - DNA, 20 bp, Artificial Sequence
  TGFBR2 target sequence 13
gtgagcaatcccccgggcga

SEQ ID NO: 132 - DNA, 20 bp, Artificial Sequence
  TGFBR2 target sequence 14
gtcgttcttcacgaggatat

SEQ ID NO: 133 - DNA, 20 bp, Artificial Sequence
  TGFBR2 target sequence 15
gccgcgtcaggtactcctgt

SEQ ID NO: 134 - DNA, 20 bp, Artificial Sequence
  TGFBR2 target sequence 16
gacgcggcatgtcatcagct

SEQ ID NO: 135 - DNA, 20 bp, Artificial Sequence
  TGFBR2 target sequence 17
gcttctgctgccggttaacg

SEQ ID NO: 136 - DNA, 20 bp, Artificial Sequence
  TGFBR2 target sequence 18
gtggatgacctggctaacag

SEQ ID NO: 137 - DNA, 20 bp, Artificial Sequence
  TGFBR2 target sequence 19
gtgatcacactccatgtggg

SEQ ID NO: 138 - DNA, 20 bp, Artificial Sequence
  TGFBR2 target sequence 20
gcccattgagctggacaccc

SEQ ID NO: 139 - DNA, 20 bp, Artificial Sequence
  TGFBR2 target sequence 21
gcggtcatcttccaggatga

SEQ ID NO: 140 - DNA, 20 bp, Artificial Sequence
  TGFBR2 target sequence 22
gggagctgcccagcttgcgc

SEQ ID NO: 141 - DNA, 20 bp, Artificial Sequence
  TGFBR2 target sequence 23
gttgatgttgttggcacacg

SEQ ID NO: 142 - DNA, 20 bp, Artificial Sequence
  TGFBR2 target sequence 24
ggcatcttgggcctcccaca

SEQ ID NO: 143 - DNA, 20 bp, Artificial Sequence
  TGFBR2 target sequence 25
gcggcatgtcatcagctggg

SEQ ID NO: 144 - DNA, 20 bp, Artificial Sequence
  TGFBR2 target sequence 26
gctcctcagccgtcaggaac

SEQ ID NO: 145 - DNA, 20 bp, Artificial Sequence
  TGFBR2 target sequence 27
gctggtgttatattctgatg

SEQ ID NO: 146 - DNA, 20 bp, Artificial Sequence
  TGFBR2 target sequence 28
ccgacttctgaacgtgcggt

SEQ ID NO: 147 - DNA, 20 bp, Artificial Sequence
  TGFBR2 target sequence 29
tgctggcgatacgcgtccac

SEQ ID NO: 148 - DNA, 20 bp, Artificial Sequence
  TGFBR2 target sequence 30
cccgacttctgaacgtgcgg

SEQ ID NO: 149 - DNA, 20 bp, Artificial Sequence
  TGFBR2 target sequence 31
ccaccgcacgttcagaagtc

SEQ ID NO: 150 - DNA, 20 bp, Artificial Sequence
  TGFBR2 target sequence 32
tcacccgacttctgaacgtg

SEQ ID NO: 151 - DNA, 20 bp, Artificial Sequence
  TGFBR2 target sequence 33
cccaccgcacgttcagaagt

SEQ ID NO: 152 - DNA, 20 bp, Artificial Sequence
  TGFBR2 target sequence 34
cgagcagcggggtctgccat

SEQ ID NO: 153 - DNA, 20 bp, Artificial Sequence
  TGFBR2 target sequence 35
acgagcagcggggtctgcca

SEQ ID NO: 154 - DNA, 20 bp, Artificial Sequence
  TGFBR2 target sequence 36
agcggggtctgccatgggtc

SEQ ID NO: 155 - DNA, 20 bp, Artificial Sequence
  TGFBR2 target sequence 37
cctgagcagcccccgaccca

SEQ ID NO: 156 - DNA, 20 bp, Artificial Sequence
  TGFBR2 target sequence 38
aacgtgcggtgggatcgtgc

SEQ ID NO: 157 - DNA, 20 bp, Artificial Sequence
  TGFBR2 target sequence 39
ggacgatgtgcagcggccac

SEQ ID NO: 158 - DNA, 20 bp, Artificial Sequence
  TGFBR2 target sequence 40
gtccacaggacgatgtgcag

SEQ ID NO: 159 - DNA, 20 bp, Artificial Sequence
  TGFBR2 target sequence 41
catgggtcgggggctgctca

SEQ ID NO: 160 - DNA, 20 bp, Artificial Sequence
  TGFBR2 target sequence 42
ccatgggtcgggggctgctc

SEQ ID NO: 161 - DNA, 20 bp, Artificial Sequence
  TGFBR2 target sequence 43
cagcggggtctgccatgggt

SEQ ID NO: 162 - DNA, 20 bp, Artificial Sequence
  TGFBR2 target sequence 44
atgggtcgggggctgctcag

SEQ ID NO: 163 - DNA, 20 bp, Artificial Sequence
  TGFBR2 target sequence 45
cggggtctgccatgggtcgg

SEQ ID NO: 164 - DNA, 20 bp, Artificial Sequence
  TGFBR2 target sequence 46
aggaagtctgtgtggctgta

SEQ ID NO: 165 - DNA, 20 bp, Artificial Sequence
  TGFBR2 target sequence 47
ctccatctgtgagaagccac

SEQ ID NO: 166 - DNA, 20 bp, Artificial Sequence
  TGFBR2 target sequence 48
atgatagtcactgacaacaa

SEQ ID NO: 167 - DNA, 20 bp, Artificial Sequence
  TGFBR2 target sequence 49
gatgctgcagttgctcatgc

SEQ ID NO: 168 - DNA, 20 bp, Artificial Sequence
  TGFBR2 target sequence 50
acagccacacagacttcctg

SEQ ID NO: 169 - DNA, 20 bp, Artificial Sequence
  TGFBR2 target sequence 51
gaagccacaggaagtctgtg

SEQ ID NO: 170 - DNA, 20 bp, Artificial Sequence
  TGFBR2 target sequence 52
ttcctgtggcttctcacaga

SEQ ID NO: 171 - DNA, 20 bp, Artificial Sequence
  TGFBR2 target sequence 53
ctgtggcttctcacagatgg

SEQ ID NO: 172 - DNA, 20 bp, Artificial Sequence
  TGFBR2 target sequence 54
tcacaaaatttacacagttg

SEQ ID NO: 173 - DNA, 20 bp, Artificial Sequence
  TGFBR2 target sequence 55
cccctaccatgactttattc

SEQ ID NO: 174 - DNA, 20 bp, Artificial Sequence
  TGFBR2 target sequence 56
ccagaataaagtcatggtag

SEQ ID NO: 175 - DNA, 20 bp, Artificial Sequence
  TGFBR2 target sequence 57
gacaacatcatcttctcaga

SEQ ID NO: 176 - DNA, 20 bp, Artificial Sequence
  TGFBR2 target sequence 58
tccagaataaagtcatggta

SEQ ID NO: 177 - DNA, 20 bp, Artificial Sequence
  TGFBR2 target sequence 59
ggtaggggagcttggggtca

SEQ ID NO: 178 - DNA, 20 bp, Artificial Sequence
  TGFBR2 target sequence 60
ttctccaaagtgcattatga

SEQ ID NO: 179 - DNA, 20 bp, Artificial Sequence
  TGFBR2 target sequence 61
catcttccagaataaagtca

SEQ ID NO: 180 - DNA, 20 bp, Artificial Sequence
  TGFBR2 target sequence 62
cacatgaagaaagtctcacc

SEQ ID NO: 181 - DNA, 20 bp, Artificial Sequence
  TGFBR2 target sequence 63
ttccagaataaagtcatggt

SEQ ID NO: 182 - DNA, 20 bp, Artificial Sequence
  TGFBR2 target sequence 64
ttttccttcataatgcactt

SEQ ID NO: 183 - protein, 326 aa, Homo sapiens
  Human IgG2 Fc
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 184 - protein, 327 aa, Homo sapiens
  Human IgG4 Fc
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 185 - DNA, 133 bp, Artificial Sequence
  SV40 poly A signal
tgctttatttgtgaaatttgtgatgctattgctttatttgtaaccattataagctgcaataaacaagttaacaacaacaattgcattcattttatgtttcaggttcagggggaggtgtgggaggttttttaaa

SEQ ID NO: 186 - DNA, 347 bp, Artificial Sequence
  MND promoter
gaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatc

SEQ ID NO: 187 - protein, 228 aa, Artificial Sequence
  Spacer
ESKYGPPCPPCPAPPVAGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 188 - protein, 164 aa, Homo sapiens
  CD3 zeta isoform 1 precursor protein
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 189 - protein, 163 aa, Homo sapiens
  CD3 zeta isoform 2 precursor protein
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR